(19) United States
(12) Patent Application Publication
(10) Pub. No.: US 2022/0142948 A1
(43) Pub. Date: May 12, 2022

(54) COMPOSITIONS AND METHODS FOR MODULATING METABOLIC REGULATORS OF T CELL PATHOGENICITY

(71) Applicants: The Broad Institute, Inc., Cambridge, MA (US); Massachusetts Institute of Technology, Boston, MA (US); The Brigham and Women's Hospital, Inc., Boston, MA (US); The Regents of the University of California, Oakland, CA (US)

(72) Inventors: Aviv Regev, Cambridge, MA (US); Chao Wang, Boston, MA (US); Vijay K. Kuchroo, Boston, MA (US); Nir Yosef, Oakland, CA (US); Allon Wagner, Oakland, CA (US); Johannes Fessler, Boston, MA (US)

(21) Appl. No.: 17/440,282

(22) PCT Filed: Mar. 18, 2020
(86) PCT No.: PCT/US2020/023399

§ 371 Date: Sep. 17, 2021

Related U.S. Application Data
(60) Provisional application No. 62/820,208, filed on Mar. 18, 2019.
(60) Provisional application No. 62/866,547, filed on Jun. 25, 2019.
(60) Provisional application No. 62/964,289, filed on Jan. 22, 2020.

(51) Int. Cl. A61K 31/132; C12N 5/0783; C12N 9/02; C12N 9/10; C12N 9/88; C12N 15/11

(52) U.S. Cl. CPC A61K 31/132; C12N 5/0636; C12N 9/0071; C12N 2506/11; C12N 9/88; C12N 15/111; C12N 9/1029

(57) ABSTRACT

The subject matter disclosed herein is generally directed to modulation of Th17 differentiation and pathogenicity by use of metabolic targets. The metabolic targets are the molecules of the polyamine pathway or glycolysis pathway. Modulation of the polyamine pathway can shift Th17 pathogenicity and shift the transcriptome of Th17 cells to a Treg or Th1 transcriptome. The polyamine analogue DFMO can be used to modulate an inflammatory response. Inhibitors of enzymes in the glycolysis pathway can shift Th17 pathogenicity.

### CROSS-REFERENCE TO RELATED APPLICATIONS

This application claims the benefit of U.S. Provisional Application Nos. 62/820,208, filed Mar. 18, 2019, 62/866,547, filed Jun. 25, 2019, and 62/964,289, filed Jan. 22, 2020. The entire contents of the above-identified applications are hereby fully incorporated herein by reference.

### STATEMENT REGARDING FEDERALLY SPONSORED RESEARCH

This invention was made with government support under Grant No.(s) MH114821, NS045937, NS30843, AI144166, A1073748, A1039671 and A1056299 awarded by the National Institutes of Health. The government has certain rights in the invention.

### REFERENCE TO AN ELECTRONIC SEQUENCE LISTING

The contents of the electronic sequence listing (BROD_2610_ST25.txt”; Size is 10 Kilobytes and it was created on Mar. 17, 2020) is herein incorporated by reference in its entirety.

### TECHNICAL FIELD

The subject matter disclosed herein is generally directed to modulation of Th17 differentiation and pathogenicity by use of metabolic targets.

### BACKGROUND

The immune system must strike a balance between mounting proper responses to pathogens and avoiding uncontrolled, autoimmune reaction. Pro-inflammatory IL-17-producing Th17 cells are a prime case in point: as a part of the adaptive immune system, Th17 cells mediate clearance of fungal infections, but they are also strongly implicated in the pathogenesis of autoimmunity (Korn et al., 2009). In mice, although Th17 cells are present at sites of tissue inflammation and autoimmunity (Korn et al., 2009), they are also normally present at mucosal barrier sites, where they maintain barrier functions without inducing tissue inflammation (Blaschitz and Raffatellu, 2010).

Interleukin (IL)-17-producing helper T cells (Th17 cells) have been identified as a distinct lineage of CD4⁺ T helper cells producing IL-17A and IL-17F and are critical drivers of autoimmune tissue inflammation in experimental autoimmune encephalomyelitis (EAE) and in other autoimmune conditions (Korn et al., 2009). In a recent study, it has been shown that the Th17 cell differentiation program is regulated through two self-reinforcing and mutually antagonistic modules of positive and negative regulators (Yosef et al., 2013). This model was supported by transcriptional silencing and genetic ablation experiments (Yosef et al., 2013), as well as by chromatin immunoprecipitation (ChIP)-seq data (Xiao et al., 2014). The positive regulators promote the Th17 cell program while inhibiting the transcriptional programs of other T helper (Th) cell lineages (Th1, Treg). This suggests that there is not only a need for positive regulators to push the differentiation into a positive direction but also for concurrent inhibition of opposing differentiation programs to achieve unidirectional T cell differentiation. Other studies also support such a mutually antagonistic design in other Th lineages (O'Shea and Paul, 2010), however, how this is achieved for Th17 cells has not been elucidated.

In humans, functionally distinct Th17 cells have been described; for instance, Th17 cells play a protective role in clearing different types of pathogens like Candida albicans (Hernandez-Santos and Gaffen, 2012) or Staphylococcus aureus (Lin et al., 2009), and promote barrier functions at the mucosal surfaces (Symons et al., 2012), despite their pro-inflammatory role in autoimmune diseases such as rheumatoid arthritis, multiple sclerosis, psoriasis systemic lupus erythematous and asthma (Waite and Skokos, 2012). Thus, there is considerable diversity in the biological function of Th17 cells and in their ability to induce tissue inflammation or provide tissue protection.

Accordingly, there exists a need for a better understanding of the dynamic regulatory network that modulates, controls, or otherwise influences T cell balance, including Th17 cell differentiation, maintenance and function, and means for exploiting this network in a variety of therapeutic and diagnostic methods.

### SUMMARY

In one aspect, the present invention provides for a method of shifting T cell balance in a population of cells comprising T cells, said method comprising contacting the population of cells with one or more agents capable of modulating the polyamine pathway. In certain embodiments, Th17 cell balance is shifted towards Treg-like cells and/or is shifted away from Th17 cells; or is shifted towards Th17 cells and/or is shifted away from Treg-like cells. In certain embodiments, Th17 cell balance is shifted towards non-pathogenic Th17 cells and/or is shifted away from pathogenic Th17 cells; or is shifted towards pathogenic Th17 cells and/or is shifted away from non-pathogenic Th17 cells.

In certain embodiments, the one or more agents capable of shifting T cell balance towards Treg-like cells and/or away from Th17 cells comprise a polyamine or polyamine analogue. In certain embodiments, the polyamine analogue is 2-(difluoromethyl)ornithine (DFMO) or a derivative thereof.

In certain embodiments, the one or more agents modulate the expression, activity or function of one or more proteins in the polyamine pathway or downstream targets thereof. In certain embodiments, the one or more agents modulate the expression, activity or function of SAT1. In certain embodiments, the one or more agents comprise Diminazene-aceturate or a derivative thereof. In certain embodiments, the one or more agents modulate the expression, activity or function of ODC1. In certain embodiments, the one or more agents comprise DFMO or a derivative thereof. In certain embodiments, the one or more agents modulate the expression, activity or function of spermidine synthase (SRM). In certain embodiments, the one or more agents comprise trans-4-methylcyclohexylamine (MCHA) or a derivative thereof. In certain embodiments, the one or more agents modulate the expression, activity or function of spermine synthase (SMS). In certain embodiments, the one or more agents comprise N-(3-aminopropyl)-cyclohexyl amine (APCHA) or a derivative thereof. In certain embodiments, the one or more agents modulate the expression, activity or function of one or more genes or gene products selected from the group consisting of JMJD3, POU2F2, POU2F1, POU5F1B, POU3F4, POU1F1, POU3F2, POU3F3, POU4F2, POU2F3, POU3F1, POU4F1, NFAT5, NFATC2, c-MAF and BATF. In certain embodiments, the one or more agents capable of shifting T cell balance towards Th17 cells and/or away from Treg-like cells comprises GSK-J1. In certain embodiments, the one or more agents capable of shifting T cell balance towards Treg-like cells and/or away from Th17 cells comprises an agonist of JMJD3.

In certain embodiments, the one or more agents comprise a small molecule, small molecule degrader, genetic modifying agent, antibody, antibody fragment, antibody-like protein scaffold, aptamer, protein, or any combination thereof. In certain embodiments, the genetic modifying agent comprises a CRISPR system, RNAi system, zinc finger nuclease system, TALE system, or a meganuclease. In certain embodiments, the CRISPR system is a Class 1 or Class 2 CRISPR system. In certain embodiments, the Class 2 system comprises a Type II Cas polypeptide. In certain embodiments, the Type II Cas is a Cas9. In certain embodiments, the Class 2 system comprises a Type V Cas polypeptide. In certain embodiments, the Type V Cas is Cas12a, Cas12b, Cas12c, Cas12d (CasY), Cas12e(CasX), or Cas14. In certain embodiments, the Class 2 system comprises a Type VI Cas polypeptide. In certain embodiments, the Type VI Cas is Cas13a, Cas13b, Cas13c or Cas13d. In certain embodiments, the CRISPR system comprises a dCas fused or otherwise linked to a nucleotide deaminase. In certain embodiments, the nucleotide deaminase is a cytidine deaminase or an adenosine deaminase. In certain embodiments, the dCas is a dCas9, dCas12 or dCas13. In certain embodiments, the CRISPR system is a prime editing system.

In certain embodiments, the population of cells comprises naïve T cells, Th1 cells and/or Th17 cells. In certain embodiments, the population of cells are in vitro cells. In certain embodiments, the population of cells is an in vivo population of cells in a subject at risk for or suffering from a disease or condition characterized by an aberrant immune response, whereby the one or more agents are used to treat the disease or condition. In certain embodiments, the population of cells are ex vivo cells obtained from a healthy donor subject or from a subject at risk for or suffering from a disease or condition characterized by an aberrant immune response. In certain embodiments, the disease is an inflammatory and/or autoimmune disorder. In certain embodiments, the inflammatory disorder is selected from the group consisting of Multiple Sclerosis (MS), Irritable Bowel Disease (IBD), Crohn's disease, ulcerative colitis, spondyloarthritides, Systemic Lupus Erythematosus (SLE), Vitiligo, rheumatoid arthritis, psoriasis, Sjögren's syndrome, diabetes, psoriasis, Irritable bowel syndrome (IBS), allergic asthma, food allergies and rheumatoid arthritis. In certain embodiments, the condition is an autoimmune response. In certain embodiments, the subject at risk for or suffering from an autoimmune response is a subject undergoing immunotherapy. In certain embodiments, the immunotherapy is checkpoint blockade therapy and/or adoptive cell transfer. In certain embodiments, the checkpoint blockade therapy comprises anti-PD1, anti-CTLA4, anti-PD-L1, anti-TIM3, anti-TIGIT, anti-LAG3, or combinations thereof. In certain embodiments, the one or more agents are administered before, during or after administering the immunotherapy. In certain embodiments, the subject undergoing immunotherapy is suffering from cancer.

In certain embodiments, the naïve T cells are differentiated into Th17 cells, Th1 cells and/or Treg cells. In certain embodiments, the one or more agents are administered to the population of cells during differentiation. In certain embodiments, the differentiation conditions comprise cell culture media supplemented with IL-6 and TGF-β1 or supplemented with IL-1β, IL-6 and IL-23. In certain embodiments, T cell differentiation is shifted towards Treg cells and/or is shifted away from Th17 cells. In certain embodiments, T cell differentiation is shifted towards Th17 cells and/or is shifted away from Treg cells. In certain embodiments, T cell differentiation is shifted towards Th1 cells and/or is shifted away from Th17 cells. In certain embodiments, T cell differentiation is shifted towards Th17 cells and/or is shifted away from Th1 cells. In certain embodiments, T cell differentiation is shifted towards non-pathogenic Th17 cells and/or is shifted away from pathogenic Th17 cells.

In another aspect, the present invention provides for a population of T cells obtained by the method according to any embodiment herein (claims 1-48). In another aspect, the present invention provides for a pharmaceutical composition comprising the population of T cells. In another aspect, the present invention provides for a method of treating a disease or condition characterized by an aberrant immune response comprising administering the pharmaceutical to a subject in need thereof.

In another aspect, the present invention provides for a method of monitoring Th17 mediated autoimmunity in a subject comprising detecting one or more polyamines in the subject, wherein increased polyamines as compared to a control indicates autoimmunity.

In another aspect, the present invention provides for a method of treating autoimmunity in a subject in need thereof, comprising monitoring Th17 mediated autoimmunity in the subject by detecting one or more polyamines in the subject; and treating the subject according to any embodiment herein when increased polyamines are detected.

In another aspect, the present invention provides for a method of shifting Th17 cell pathogenicity in a population of cells comprising T cells, said method comprising contacting the population of cells with one or more agents capable of modulating a reaction of the glycolysis pathway according to Table 1 or 2. In certain embodiments, the one or more agents modulate expression, activity, or function of a gene or gene product selected from the group consisting of: G6PD, PKM, Aldo, PFKM, TA, G6PC, PGAM, GK, ENO1, PCK1, TPI1, PGK1, GAPDHS, PDHA1, and GPD1. In certain embodiments, the one or more agents is selected from the group consisting of 2,5-Anhydro-D-glucitol-1,6-diphosphate, S-HD-CoA, DHEA, TX1, Gimeracil, Shikonin, Pyruvate Kinase Inhibitor III, 2,3-Dihydroxypropyl dichloroacetate (DCA), 2,9-Dimethyl-BC, Koningic acid, CBR-470-1, EGCG, SF2312, PhAh, ENOblock, 3-MPA, and 6,8-Bis(benzylthio)octanoic acid.

In certain embodiments, the one or more agents comprise a small molecule, small molecule degrader, genetic modifying agent, antibody, antibody fragment, antibody-like protein scaffold, aptamer, protein, or any combination thereof. In certain embodiments, the genetic modifying agent comprises a CRISPR system, RNAi system, zinc finger nuclease system, TALE system, or a meganuclease. In certain embodiments, the CRISPR system is a Class 1 or Class 2 CRISPR system. In certain embodiments, the Class 2 system comprises a Type II Cas polypeptide. In certain embodiments, the Type II Cas is a Cas9. In certain embodiments, the Class 2 system comprises a Type V Cas polypeptide. In certain embodiments, the Type V Cas is Cas12a, Cas12b, Cas12c, Cas12d (CasY), Cas12e(CasX), or Cas14. In certain embodiments, the Class 2 system comprises a Type VI Cas polypeptide. In certain embodiments, the Type VI Cas is Cas13a, Cas13b, Cas13c or Cas13d. In certain embodiments, the CRISPR system comprises a dCas fused or otherwise linked to a nucleotide deaminase. In certain embodiments, the nucleotide deaminase is a cytidine deaminase or an adenosine deaminase. In certain embodiments, the dCas is a dCas9, dCas12 or dCas13. In certain embodiments, the CRISPR system is a prime editing system.

In certain embodiments, the population of cells comprises naïve T cells, Th1 cells and/or Th17 cells. In certain embodiments, the population of cells are in vitro cells. In certain embodiments, the population of cells is an in vivo population of cells in a subject at risk for or suffering from a disease or condition characterized by an aberrant immune response, whereby the one or more agents are used to treat the disease or condition. In certain embodiments, the population of cells are ex vivo cells obtained from a healthy donor subject or from a subject at risk for or suffering from a disease or condition characterized by an aberrant immune response. In certain embodiments, the disease is an inflammatory and/or autoimmune disorder. In certain embodiments, the inflammatory disorder is selected from the group consisting of Multiple Sclerosis (MS), Irritable Bowel Disease (IBD), Crohn's disease, ulcerative colitis, spondyloarthritides, Systemic Lupus Erythematosus (SLE), Vitiligo, rheumatoid arthritis, psoriasis, Sjögren's syndrome, diabetes, psoriasis, Irritable bowel syndrome (IBS), allergic asthma, food allergies and rheumatoid arthritis. In certain embodiments, the condition is an autoimmune response. In certain embodiments, the subject at risk for or suffering from an autoimmune response is a subject undergoing immunotherapy. In certain embodiments, the immunotherapy is checkpoint blockade therapy and/or adoptive cell transfer. In certain embodiments, the checkpoint blockade therapy comprises anti-PD1, anti-CTLA4, anti-PD-L1, anti-TIM3, anti-TIGIT, anti-LAG3, or combinations thereof. In certain embodiments, the one or more agents are administered before, during or after administering the immunotherapy. In certain embodiments, the subject undergoing immunotherapy is suffering from cancer.

In certain embodiments, the naïve T cells are differentiated into Th17 cells. In certain embodiments, the one or more agents are administered to the population of cells during differentiation. In certain embodiments, the differentiation conditions comprise cell culture media supplemented with IL-6 and TGF-β1 or supplemented with IL-1β, IL-6 and IL-23. In certain embodiments, T cell differentiation is shifted towards non-pathogenic Th17 cells and/or is shifted away from pathogenic Th17 cells.

In another aspect, the present invention provides for a population of T cells obtained by the method according to any embodiment herein (claims 54-83). In another aspect, the present invention provides for a pharmaceutical composition comprising the population of T cells. In another aspect, the present invention provides for a method of treating a disease or condition characterized by an aberrant immune response comprising administering the pharmaceutical composition to a subject in need thereof.

In another aspect, the present invention provides for a data driven method of detecting metabolic target genes and pathways comprising: providing single cell RNA-seq reads obtained from a population of cells or an RNA library from multiple cells, wherein each single cell represents an observation, and the number of observations allows statistical power to discern statistically significant metabolic targets; providing metabolic data comprising the metabolic reactions in the population of cells; simulating the metabolic fluxes at the single-cell level by projecting the transcriptomic profile onto the metabolic network, thereby producing a quantitative metabolic profile of each cell. In certain embodiments, spatial, temporal or lineage delineated RNA-seq data is used to identify the metabolic state in single cells across a tissue, over time or in a cell lineage. In certain embodiments, the method comprises treating a population of cells with a drug for use in identifying metabolic adaptation to the drug. In certain embodiments, the method comprises predicting targets that will shift a population of cells in one state to another state. In certain embodiments, the state is shifted towards Treg-like cells and/or is shifted away from Th17 cells; or towards Th17 cells and/or is shifted away from Treg-like cells; or towards non-pathogenic Th17 cells and/or is shifted away from pathogenic Th17 cells; or towards pathogenic Th17 cells and/or is shifted away from non-pathogenic Th17 cells. In certain embodiments, the method is used to determine resistance to a drug, wherein the method comprises determining metabolic pathways modulated in resistant subjects as compared to sensitive subjects. In certain embodiments, the method comprises analyzing single cells obtained from a diseased tissue for use in determining metabolic shifts in disease. In certain embodiments, the disease comprises a degenerative disease, cancer, a metabolic disease, aging, autoimmunity or inflammation. In certain embodiments, the disease comprises cardiovascular disease. In certain embodiments, the disease comprises diabetes. In certain embodiments, the single cells comprise cells from an animal, plant, or bacteria. In certain embodiments, the method comprises identifying metabolic shifts in a host cell contacted with a microbiome (e.g., symbiotic microbial cells harbored by a host organism consisting of trillions of microorganisms (also called microbiota or microbes) of thousands of different species including not only bacteria, but fungi, parasites, and viruses).

In another aspect, the present invention provides for a population of T cells obtained by the method according to any embodiment herein. In another aspect, the present invention provides for a pharmaceutical composition comprising the population of T cells according to any embodiment herein. In another aspect, the present invention provides for a method of treating a disease or condition characterized by an aberrant immune response comprising administering the pharmaceutical composition of any embodiment herein to a subject in need thereof.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1D—Prediction of metabolic space associated with Th17 cell pathogenicity. FIG. 1A shows heatmaps of metabolic gene expression and metabolic reactions in Th17 cells and principal component analysis of the Th17 cells using two metabolic principal components (PC2-glycolysis and PC1-fatty acid activation). FIG. 1B shows heat map of pathogenic and non-pathogenic Th17 gene expression. FIG. 1C shows a plot using COMPASS to identify metabolic pathways relevant in Th17 cell pathogenicity. FIG. 1D shows top ranking genes by association with pathogenicity in WT Th17 cells and their associated metabolic pathways. Genes at the top have a positive association with pathogenicity and genes at the bottom have a negative association.

FIG. 2A-2F—Fluxomics and metabolomics analysis validate the association of polyamine pathway with pathogenic Th17 cells. FIG. 2A. Results of metabolomics shown as a heatmap of polyamine pathway molecule levels during pathogenic and non-pathogenic Th17 cell differentiation. FIG. 2B. Results of fluxomics shown as bar graphs of production of putrescine (left) and acetyl-spermidine (right). FIG. 2C. Diagram showing the polyamine pathway. FIG. 2D. Heat map showing results of untargeted metabolomics using mass spectrometry of metabolites in naïve, pathogenic Th17 and non-pathogenic Th17 cells. FIG. 2E. Results of fluxomics using C13 labeled precursors to the polyamine pathway shown as bar graphs of products generated. (Fluxomics using C13 labeled precursors to the polyamine pathways suggested alternative usage of the polyamine pathway by pathogenic and non-pathogenic Th17 cells. Pathogenic Th17 cells turn arginine into L-citruline (produce more NO in the process) and polyamines) FIG. 2F. Results of fluxomics using C13 labeled precursors to the polyamine pathway shown as bar graphs of products generated. (Fluxomics using C13 labeled precursors to the polyamine pathways suggested alternative usage of the polyamine pathway by pathogenic and non-pathogenic Th17 cells. D). Non-Pathogenic Th17 cells turn L-citruline into Arginine and creatinine)

FIG. 3A-3L—Polyamines and polyamine analogues can alter Th17 cell differentiation and function. FIG. 3A. Schematic showing inhibition of the polyamine pathway using 2-(difluoromethyl)ornithine (DFMO). FIG. 3B. FACS plots and bar graphs showing that IL-17 positive CD4 T cells are decreased after DFMO treatment. FIG. 3C. Quantitative real time PCR showing that IL-17 is decreased in CD4 T cells after DFMO treatment. FIG. 3D. Bar graphs showing that the addition of putrescine rescues the effect of DFMO in CD4 T cells. FIG. 3E. Graph showing that treatment of an EAE mouse model with DFMO decreases the EAE score and a bar graph showing that DFMO treatment increases FoxP3+CD4 cells (Tregs). FIG. 3F. Graph showing that treatment of an EAE mouse model with DFMO decreases H3 incorporation into antibodies after MOG inoculation. FIG. 3G. FACS analysis of non-pathogenic and pathogenic Th17 cells after treatment with polyamines. (top) FACS showing IL-17 and IL-10 positive cells. (bottom) graphs showing IL-17, IL-10 and IL-2 positive cells. FIG. 3H. FACS analysis of non-pathogenic and pathogenic Th17 cells (wild type and SAT1 KO) after treatment with DFMO. (top) FACS showing IL-17 and CD4 positive cells and graphs showing IL-17 positive cells. (bottom) RORgt expression and mean fluorescence intensity (MFI) in both pathogenic and non-pathogenic Th17 cells. FIG. 3I. Bar graphs showing protein expression of the indicated cytokine in pathogenic Th17 cells (top) and non-pathogenic Th17 cells (bottom) after treatment with DFMO. FIG. 3J. FACS plots and bar graphs showing increase in FoxP3 CD4 T cells (Tregs) in nonpathogenic Th17 cells after DFMO treatment. FIG. 3K. Bar graphs showing that the addition of putrescine rescues the effect of DFMO in pathogenic and non-pathogenic Th17 cells. FIG. 3L. Bar graphs showing that the addition of putrescine rescues the increase in FoxP3 CD4 T cells (Tregs) in nonpathogenic Th17 cells after DFMO treatment.

FIG. 4A-4G—Inhibition of the polyamine pathway transitions Th17 cells into a Treg-like transcriptome. FIG. 4A. Principle component analysis of the indicated cells treated with DFMO or vehicle. FIG. 4B. The log fold change in expression and Venn diagram of Th17 specific genes and Treg specific genes after treatment of Th17 cells with DFMO. FIG. 4C. Plots of DFMO down and up genes (fold change) in non-pathogenic (top) and pathogenic (bottom) Th17 cells (Th17 and Treg specific and shared genes are labeled). FIG. 4D. Bar graphs showing relative expression of IL17A, IL17F and Foxp3 in non-pathogenic (top) and pathogenic (bottom) Th17 cells after DFMO treatment. FIG. 4E. Plot of DFMO down and up chromatin associated genes (fold change) in pathogenic Th17 cells. FIG. 4F. Plots showing DFMO effect on chromatin accessibility of Th17 and iTreg genes in iTregs, non-pathogenic Th17 cells, and pathogenic Th17 cells. FIG. 4G. Plots showing chromatin accessibility as compared to gene expression illustrating the effect of DFMO on Th17 (bottom) and iTreg (top) ATAC peaks in iTregs, non-pathogenic Th17 cells, and pathogenic Th17 cells.

FIG. 5A-5B—DFMO reduces accessibility in regions specific to Th17 (vs. Treg). FIG. 5A. ATAC-seq of Th17 specific chromatin regions with and without DFMO treatment. FIG. 5B. Bar graph showing less and more accessible regions and plot showing shift of Th17 regions and regions shared between Th17 and Tregs. Th17 shifted more.

FIG. 6A-6C—Conditional deletion of Sat1 in T cells alleviates EAE severity and promotes frequency of Tregs. FIG. 6A. Bar graph showing a decrease in SAT1 after DFMO treatment. FIG. 6B. Graphs showing indicated polyamine abundance in WT and SAT1 KO T cells. FIG. 6C. (left) Graph showing the mean clinical score after EAE induction of the indicated mice. (right) Graph showing the percentage of FoxP3+ T cells in WT and SAT1 KO T cells.

FIG. 7—Suppression of IL-17 by DFMO is dependent on the timing of DFMO treatment. FACS and bar graph showing the percentage of IL-17+CD4 T cells after no DFMO treatment (−/−), after treatment at the time of differentiation (DFMO/−), after treatment at the time of differentiation and the expansion phase (DFMO/DFMO), and after treatment at only the expansion phase (−/DFMO). Time of differentiation (DFMO at Day 1-3) and expansion phase (DFMO at Day 4-5) is indicated.

FIG. 8A-8D—DFMO promotes IL-21, IL-22 and IL9 expression. FIG. 8A. Bar graphs showing protein expression of the indicated cytokine in pathogenic Th17 cells (wild type and SAT1 KO) after treatment with DFMO. FIG. 8B. Bar graphs showing protein expression of the indicated cytokine in non-pathogenic Th17 cells (wild type and SAT1 KO) after treatment with DFMO. FIG. 8C. Bar graphs showing quantitative PCR results for the indicated protein in pathogenic Th17 cells (wild type and SAT1 KO) after treatment with DFMO. FIG. 8D. Bar graphs showing quantitative PCR results for the indicated protein in non-pathogenic Th17 cells (wild type and SAT1 KO) after treatment with DFMO.

FIG. 9A-9B—DFMO does not seem to alter pStat3. FIG. 9A. (top) Plots showing pSTAT3 expression under each condition indicated. (bottom) STAT3 and pSTAT3 expression at the indicated time points. FIG. 9B. Bar graphs showing expression of the indicated proteins in pathogenic Th17 cells, non-pathogenic Th17 cells, iTreg cells, and Th0 cells after treatment with DFMO.

FIG. 10—DFMO promotes H3K4, H3K27, H3K9 trimethylation. Graphs showing the mean fluorescence intensity (MFI) for the indicated targets in pathogenic Th17 cells, non-pathogenic Th17 cells and iTreg cells treated+/− with DFMO. Also, shown are plots indicating the levels of H3, H3 acetylated at lysine 4, and H3 trimethylated at lysine 4 in non-pathogenic Th17 cells treated+/− with DFMO.

FIG. 11—DFMO and polyamines alter enzymes of the polyamine pathway and DFMO treatment suppresses Sat1. (top) Graphs showing the relative expression of ASS1 and SSAT in pathogenic and non-pathogenic Th17 cells after treatment with putrescine and arginine. (bottom) Bar graphs showing quantitative PCR results for the indicated protein in cells treated with DFMO or indicated polyamine.

FIG. 12A-12B—Perturbation of Sat1 partially mimics and has an additive effect with DFMO on Th17 cell function. FIG. 12A. Relative expression of N-acetylspermidine and argininosuccinate in pathogenic and non-pathogenic Th17 cells (wild type and SAT1 KO). FIG. 12B. Relative expression of N-acetylspermidine in pathogenic and non-pathogenic Th17 cells treated with indicated polyamines (wild type and SAT1 KO). FIG. 12C. (top) cell metabolism assay. (bottom) Heatmap showing differentially expressed genes.

FIG. 13A-13B—Perturbation of Sat1 partially mimics and has an additive effect with DFMO on Th17 cell function. FIG. 13A. (left) Graph showing the mean clinical score after EAE induction of the indicated mice. (right) Graph showing CNS histology score for the mice. (bottom) Table showing quantification of data. FIG. 13B. (top)³H incorporation assay after immunization with MOG. (bottom) MOG response assay.

FIG. 14—FACS analysis of FoxP3 and RORgt expressing cells.

FIG. 15A-15K—FIG. 15A. Gene expression in pathogenic and non-pathogenic Th17 cells. FIG. 15B. Polyamines correlate with the pathogenic signature. FIG. 15C. Polyamines correlate with the pathogenic signature. FIG. 15D. Polyamine pathway. FIG. 15E. Polyamine expression in Th17 cells. FIG. 15F. C¹³ incorporation in newly synthesized polyamines in Th17 cells. FIG. 15G. Gene expression in pathogenic and non-pathogenic Th17 cells. FIG. 15H. Gene expression in pathogenic and non-pathogenic Th17 cells. FIG. 15I. Polyamines correlate with the pathogenic signature. FIG. 15J. Relative expression of enzymes in T cells. FIG. 15K. Polyamine concentration in non-pathogenic Th17 cells, pathogenic Th17 cells and iTreg cells.

FIG. 16A-16L—FIG. 16A. DFMO inhibits the polyamine pathway. FIG. 16B. DFMO effect on polyamine enzymes. FIG. 16C. Effect of Sat1 expression on polyamine expression. FIG. 16D. Effect of Sat1 expression on EAE and CNS infiltrate. FIG. 16E. Effect of Sat1 expression on proliferation in a MOG assay. FIG. 16F. Effect of Sat1 expression on the percentage of FoxP3 T cells. FIG. 16G. Effect of Sat1 expression on cytokine production in a MOG assay. FIG. 16H. Graph showing that treatment of an EAE mouse model with DFMO decreases the EAE score. FIG. 16I. Graph showing that treatment of an EAE mouse model with DFMO decreases H3 incorporation into antibodies after MOG inoculation. FIG. 16J. Bar graph showing that DFMO treatment increases FoxP3+CD4 cells (Tregs). FIG. 16K. Quantitative RT-PCR showing expression of polyamine enzymes in Th17 cells after treatment with DFMO. FIG. 16L. Bar graphs showing DFMO and a polyamine rescues the decrease in IL-17 and increase in Foxp3 T cells.

FIG. 17A-17C—FIG. 17A. Heatmaps showing expression of metabolites in the indicated Th17 cells. Metabolites are different between non-pathogenic and pathogenic Th17 cells. FIG. 17B. Graphs showing the levels of polyamines in the indicated Th17 cells and media. FIG. 17C. Graphs showing changes over time of guanidinoacetic acid and creatine in non-pathogenic and pathogenic Th17 cells.

FIG. 18A-18D—FIG. 18A. DFMO effect on polyamine concentration in iTregs, non-pathogenic Th17 cells, and pathogenic Th17 cells. FIG. 18B. Bar graphs showing production of indicated cytokines in pathogenic (top) and non-pathogenic (bottom) Th17 cells after DFMO treatment. FIG. 18C. Graphs showing amount of indicated phosphorylated transcription factors in pathogenic and non-pathogenic Th17 cells after DFMO treatment. FIG. 18D. Quantitative RT-PCR showing expression of polyamine enzymes in Th17 cells after treatment with DFMO.

FIG. 19A-19E—FIG. 19A. Principle component analysis of the indicated cells treated with DFMO or vehicle. FIG. 19B. Plots showing chromatin accessibility of non-pathogenic Th17 and pathogenic Th17 genes. FIG. 19C. Correlation between RNA-seq and ATAC-seq peaks for Th17 specific genes. FIG. 19D. Correlation between RNA-seq and ATAC-seq peaks for iTreg specific genes. FIG. 19E. Enrichment of accessible transcription factor motifs in non-pathogenic Th17 cells for Th17 specific and iTreg specific genes.

FIG. 20A-20H—FIG. 20A. Schematic showing inhibition of the polyamine pathway using specific small molecules targeting polyamine enzymes. FIG. 20B. FACS plots and bar graphs showing that IL-17 positive CD4 T cells are decreased after DFMO treatment. FIG. 20C. Bar graphs showing protein expression of the indicated cytokine in pathogenic Th17 cells (top) and non-pathogenic Th17 cells (bottom) after treatment with DFMO. FIG. 20D. Graphs showing that DFMO does not alter RORgt levels in Th17 cells. FIG. 20E. FACS plots and bar graph showing increase in FoxP3 CD4 T cells after DFMO treatment. FIG. 20F. Graphs showing that inhibition of the polyamine pathway with the indicated small molecule reduces IL-17 T cells and increases IL-9 and Foxp3 T cells. FIG. 20G. Bar graphs showing that the addition of putrescine rescues the effect of DFMO in non-pathogenic Th17 cells. FIG. 20H. Bar graphs showing that the addition of putrescine rescues the effect of diminazene aceturate in non-pathogenic Th17 cells.

FIG. 21A-21I—FIG. 21A. Principle component analysis of the indicated cells treated with DFMO or vehicle. FIG. 21B. The log fold change in expression of Th17 specific and Treg specific genes after treatment of Th17 cells with DFMO. FIG. 21C. (top) Plots of DFMO down and up genes (fold change) in non-pathogenic (left) and pathogenic (right) Th17 cells. (bottom) Bar graphs showing relative expression of IL17A, IL17F and Foxp3 in non-pathogenic and pathogenic Th17 cells after DFMO treatment. FIG. 21D. Plot of DFMO Tn5 cuts at chromatin associated gene loci (fold change) in non-pathogenic Th17 cells. FIG. 21E. Plots showing DFMO effect on chromatin accessibility of Th17 and iTreg genes in iTregs, non-pathogenic Th17 cells, and pathogenic Th17 cells. FIG. 21F. (top) ATAC-seq of IL-17 specific chromatin regions with and without DFMO treatment. (bottom) ATAC-seq of IL-23r specific chromatin regions with and without DFMO treatment. FIG. 21G. ATAC-seq of Foxp3 specific chromatin regions with and without DFMO treatment. FIG. 21H. Enrichment of accessible transcription factor motifs in pathogenic Th17 cells for Th17 specific and iTreg specific genes. FIG. 21I. Graphs showing Foxp3+ T cells and IL-17+ T cells after DFMO treatment in wildtype cells and C-MAF knockout cells.

FIG. 22A-22C—Prediction of metabolic space associated with Th17 cell pathogenicity using COMPASS. FIG. 22A Computation of Compass scores matrix. Compass leverages prior knowledge on metabolic topology and stoichiometry (encoded in a GEM, see main text) to analyze single-cell RNA-Seq expression. Briefly, it computes a reaction-penalties matrix which is the input to a set of flux-balance linear programs that produce a score for every reaction in every cell, namely the Compass score matrix. FIG. 22B To compute the reaction penalties matrix, Compass allows soft information sharing between a cell and its k-nearest neighbors to mitigate technical noise in single-cell library preparation. FIG. 22C. Downstream analysis of the score matrix. Rows are hierarchically clustered into meta-reactions, which are data-driven “mini-pathways”. The scores are then amenable to common genomics procedures including differential expression of meta-reactions, detecting meta-reactions correlating with a phenotype of interest, dimensionality reduction, and network analysis.

FIG. 23A-23F—FIG. 23A. The experimental system. Naive CD4+ T cells are collected and differentiated into Th17p or Th17n cells, which are IL-17+ T cells that cause severe or mild-to-none CNS autoimmunity upon adoptive transfer. Th17nu cells are Th17n cells which were not sorted by IL-17 and exhibit higher variability. FIG. 23B. The first principal component (PC1) of the Compass scores matrix of Th17nu cells is highly correlated with overall metabolic activity and Th17 differentiation time course signature (defined in Methods). FIG. 23C. PC2 represents a metabolic axis concerning a cell's strategy for ATP production. Low/high values correspond to a preference towards aerobic glycolysis or beta-oxidation, respectively. Cells are shaded by the ratio between of glucose transporters (GLUT) to carnitine palmitoyltransferase 1 (CPT1) which is a transcriptome-based metric for a cell's affinity towards aerobic glycolysis or beta-oxidation. FIG. 23D. Schematic showing metabolic reactions predicted by COMPASS. FIG. 23E. Plot showing metabolic pathways correlated with Th17 pathogenicity and enzymes involved in the reactions associated with the pathways. FIG. 23F. Dots are single metabolic reactions, and axes denote their correlation with the pathogenic signature in the Th17nu and Th17n groups. Every reaction is assigned a combined Fisher p-value of the two p-values measuring the significance of the correlation with the two axes.

FIG. 24A-24G—FIG. 24A. Schematic showing the reactions in the glycolysis pathway positively correlated with Th17 pathogenicity. Shown are the top correlating genes and drugs targeting the indicated reactions. FIG. 24B. FACS analysis of non-pathogenic and pathogenic Th17 cells positive for IL-17 (top) and IL-2 (bottom) after treatment with the indicated drug in parenthesis targeting the indicated enzyme. FIG. 24C. Glycolysis and adjacent metabolic pathways. The highlighted magenta and green reactions are the two predicted to be most correlated and anti-correlated with Th17 pathogenicity, respectively. Where only one direction of the reaction was predicted, the other direction is shown with a dotted line. Reported inhibitors of these reactions are denoted (39). FIG. 24D. Effects of inhibiting candidate genes on Th17 cytokines as measured by flow cytometry are shown. Naive T cells are differentiated under pathogenic (Th17p) and non-pathogenic (Th17n) Th17 cell conditions (materials and methods) in the presence of control solvent or inhibitors. Cells were pre-labeled with division dye and RNA expression is reported for cells that have gone through one division (dl) to exclude arrested cells. FIG. 24E. PCA of bulk RNA-Seq of dl Th17 cells. FIG. 24F-FIG. 24G. pro-pathogenic and pro-regulatory genes are decided by differential expression of Th17p vs. Th17n, respectively. EGCG promotes the pro-pathogenic module and suppresses the pro-regulatory module in Th17n evidenced by (F) volcano and (G) distribution of log fold-change values of EGCG treated vs. untreated Th17n cells. DHEA does not induce an opposite effect and works through another mechanism.

FIG. 25A-25I—FIG. 25A. Graph showing the effect of the differential expression gene count and false discovery rate. FIG. 25B. Principal component analysis of wildtype (wt) and pyruvate dehydrogenase kinase 4 (PDK4)−/− Th17 cells. The plot is overlayed based on genotype or signature. The gluconeogenesis signature is expressed higher in wt. The oxidative phosphorylation signature is expressed higher in wt. The melanogenesis signature is expressed higher in PDK4−/−. FIG. 25C. Plot showing differentially expressed genes between wt and PDK4−/− in pathogenic and non-pathogenic Th17 cells. FIG. 25D. Plot showing differentially expressed signatures between wt and PDK4−/− in pathogenic and non-pathogenic Th17 cells. FIG. 25E. Schematic showing the reactions in the glycolysis pathway positively correlated with Th17 pathogenicity. PDK4 is shown in the pathway. FIG. 25F. Plot showing enzymes in the indicated pathways and biserial ranks for WT and PDK4-T cells. FIG. 25G. Plot showing body weight between wt and PDK4−/− mice induced for colitis. FIG. 25H. Graphs showing colon length and colitis score between wt and PDK4−/− mice induced for colitis. FIG. 25I. Histology of colons from wt and PDK4−/− mice induced for colitis.

FIG. 26—Plot showing the correlation of each step in the glycolysis pathway with the Th17 pathogenic signature. The genes associated with each reaction are shown above the glycolytic step.

FIG. 27A-27E—FIG. 27A. 2d UMAP projection of reaction-to-reaction cosine distances. FIG. 27B. (left) Th17p and Th17n divert glucose-derived 13C into glycolysis and TCA metabolites, respectively. (right) EGCG disturbs 3PG and PEP in Th17p and 2PG in Th17n. FIG. 27C. Bar graphs showing percent C13 incorporation in the metabolites after EGCG treatment. FIG. 27D. Plot showing enzymes in the indicated pathways and fold change between WT and EGCG treated T cells. FIG. 27E. Heat map showing that EGCG differentially affects Th17p and Th17n glycolysis and serine biosynthesis transcripts in bulk RNA-Seq.

FIG. 28A-28C—2D2 TCR-transgenic Th17 cells were adoptively transferred after activation ex vivo in the presence of an inhibitor or vehicle. In accordance with computational prediction, EGCG exacerbates EAE induced by 2D2 Th17n. DHEA alleviates EAE induced by Th17p. FIG. 28A. Clinical outcome measured by EAE score (40). FIG. 28B. histological scores. FIG. 28C. EGCG-treated Th17n cells, unlike Th17n untreated or Th17p, induce Wallerian degeneration in proximal spinal nerve roots.

FIG. 29. Graph showing the number of reactions and empirical CDF.

FIG. 30A-30G—FIG. 30A. UMAP plots showing data driven metabolic pathways in single cells. FIG. 30B. UMAP plots showing data driven metabolic pathways in single cells. FIG. 30C. UMAP analysis showing pro-pathogenic and pro-regulatory single cells. FIG. 30D. Plot showing metabolic pathways correlated with Th17 pathogenicity. FIG. 30E. Volcano plots of log fold-change values of EGCG and DHEA treated vs. untreated Th17 cells. FIG. 30F. Distribution of log fold-change values of EGCG and DHEA treated vs. untreated Th17 cells. FIG. 30G. Plot showing enzymes in the indicated pathways and fold change between WT and DHEA treated T cells.

FIG. 31A-31B—FIG. 31A. Heat map showing EGCG differentially expressed genes associated with the indicated categories. FIG. 31B. Heat map showing DHEA differentially expressed genes associated with the indicated categories.

FIG. 32—Heat map showing DHEA differentially expressed genes associated with the indicated metabolic pathways.

FIG. 33—Graph showing the incorporation of C13 into serine in Th17 cells.

FIG. 34A-34H—Prediction and metabolic validation of the polyamine pathway as a candidate in regulating Th17 cell function. (A-B) Standard single-cell RNAseq analysis of Th17 cells Applicants published in [24]. Briefly, IL-17.GFP+ cells were isolated from pathogenic Th17 cells (Th17p, IL-1b+IL-6+IL-23) and non-pathogenic Th17 cells (Th17n, TGFb+IL-6) differentiated in vitro. A, Histogram of a transcriptional pathogenicity score per cell, based on [14]; B, Gene expression heatmap of top metabolic genes associated with Th17 cell pathogenicity as Applicants qualified in [14]. Marker genes associated with pro-inflammatory (ICOS, STAT4, LRMP, IL22, LAG3, GZMB, CCL5, CXCL3, CSF2, LGALS3, TBX21, CASP1, CCL4 and CCL3) or pro-regulatory (MAF, IL9, AHR, IKZF3, IL6ST and IL10) programs were used to compute the pathogenicity score, respectively, other genes are metabolic. Cell are ordered by the ranked pathogenicity score; (B-C) Compass analysis of scRNAseq of Th17 cells. C, meta-reactions (data-driven clusters reactions with similar Compass scores across cells, with median of two reactions per meta-reaction; STAR Methods) differential activity between Th17p and Th17n conditions is assessed via Benjamini-Hochberg (BH)-adjusted Wilcoxon rank sum p value, signed by the direction of change and by the Spearman correlation of their Compass scores with cell pathogenicity scores, across all cells. Grey dots represent meta-reactions containing at least one metabolic reaction that appear in network of panel D. The meta-reaction labelled “polyamine metabolism” contains uptake of putrescine, spermidine, spermidine monoaldehyde, spermine monoaldehyde, and 4-aminobutanal from the extracellular compartment, and the conversion of 4-aminobutanal to putrescine. D, a metabolic network that is preferentially active in the pro-regulatory (Th17n) state based on Compass results. Grey arrows represent reactions that were predicted to be significantly associated with the Th17n program (BH-adjusted p<0.1 for their meta-reaction, dashed line for borderline significance, BH-adjusted p<0.12), black arrows represent reactions that were not significantly different between Th17p and Th17n; (E) Schematic of the polyamine pathway based on KEGG; SAM: S-Adenosyl-Methionine; SAH: S-Adenosyl-Homocysteine. GABA: gamma-aminobutyric acid. (F-H) validation of the polyamine pathway. Th17n and Th17p cells are differentiated as in (A) and harvested at 48h for qPCR (F) and 68h for metabolomics (g-h). F, qPCR validation of rate-limiting enzymes in polyamine metabolism ASS1, ODC1 and SAT1; G, Total polyamine content measured by ELISA; H, Abundance of metabolites in the polyamine pathway are reported as measured by LC/MS metabolomics.

FIG. 35A-35I—Chemical and genetic interference with the polyamine pathway suppress canonical Th17 cell cytokines. (A), Polyamine pathway overview depicting inhibitors of ODC1 (DFMO), SRM (MCHA), SMS (APCHA) and SAT1 (Diminazene aceturate). (B-E) The effects of DFMO on Th17n and Th17p cells differentiated as in FIG. 1. DFMO were added at the time of differentiation cytokines. All analysis performed on day 3. B-C, Flow cytometric analysis of intracellular cytokines (B) and secreted cytokines by legendplex (C); D, Flow cytometric analysis of transcription factor expression in Th17n and Th17p; E, Flow cytometric analysis of Foxp3 expression in Th17n. (F) Comparison of IL-17A, IL-9 and FoxP3 expression following treatment with Ctrl, DFMO, MCHA, APCHA or Diminazene aceturate in in vitro differentiated Th17n cells. (GH) The rescue effect of adding putrescine on inhibitors to ODC1 (G) or SAT1 (H). Expression of IL-17A and FoxP3 following combinational treatment of DFMO (G) or Diminazene aceturate (H) with or without the addition of 2.5 mM Putrescine. (I) The effects of genetic perturbation of ODC1. Th17n and Th17p cells are generated from naïve T cells isolated from WT or ODC1^{-/-} mice and treated with control or DFMO in combination with 0 or 2.5 mM Putrescine. Flow cytometric analysis of intracellular IL-17 and Foxp3 are shown. Each dot represents biological replicates performed with different mice. All statistical analyses are performed using pair-wise comparison or one-way anova.

FIG. 36A-36G—DFMO treatment promotes Treg-like transcriptome and epigenome. (A-C) Th17n, Th17p and iTreg cells were differentiated and harvested at 68h for live cell sorting and population RNAseq. A, PCA plot showing in vitro differentiated Th17n, Th17p and iTregs in the presence (lighter shade) or absence of DFMO. B, Volcano plots and qPCR validation (continued) showing affected genes by DFMO treatment in Th17n and Th17p cells. Th17 and iTreg specific genes (darker shading) are highlighted. C, Histograms showing the effects of DFMO on iTreg, Th17n and Th17p transcriptome. Transcriptome space is divided into those up-regulated in Th17 cells, Treg or neither. (D-F) Th17n and iTreg cells were differentiated and harvested at 68h for live cell sorting and population ATAC-seq. D, Histograms showing the effects of DFMO on chromatin accessibility as measured by ATAC-seq. The accessibility regions are divided into those more accessible in Th17 cells, Treg or neither. E, IGV plots of H17 regions. Regions significantly altered (DESeq2, BH-adjusted p<0.05) by DFMO treatment and binding sites for RORγt [32] are highlighted. F, Motif enrichment analysis of in vitro differentiated Th17n in the presence or absence of DFMO for iTreg specific genes. (G), Cells were cultured under Th17n condition as in FIG. 2 with DFMO or solvent control (water), replated to rest at 68h in new plate and harvested at 120h for analysis of intracellular Foxp3 expression and IL-10 expression in supernatant.

FIG. 37A-37I—Targeting ODC1 and SAT1 alleviate EAE. (A) Schematics of the polyamine pathway; (B-D) The effects of chemical inhibition of ODC1 by DFMO on EAE. Wildtype mice were immunized with MOG₃₅₋₅₅/CFA to induce experimental autoimmune encephalomyelitis and followed for clinical scoring. DFMO were provided in drinking water from day 0 for 10 days in experimental group. B, Clinical score over time. Graph shows pooled results from 3 independent experiments. C, Antigen-specific cell proliferation is measured by thymidine incorporation after culturing cells isolated from draining lymph node of mice (d15 post immunization) with increasing dose of MOG₃₅₋₅₅ peptide for 3 days. D, Flow cytometric analysis of intracellular Foxp3 expression in T cells isolated from CNS at day 15 post immunization. (E-I) The effects of genetic perturbation of SAT1. E, The effects of SAT1 deficiency on metabolome. Abundance of metabolites in the polyamine pathway were determined by LC/MS based metabolomics. Th17n and Th17p cells were differentiated in vitro from naïve cells isolated from WT or SAT1^{-/-} mice. (F-I) The effects of SAT1 deficiency on EAE. EAE were induced as in (B) in wildtype and SAT1^{fl/fl}CD4^{cre} mice. F, Clinical score (left) and histological score (right) showing the number of CNS infiltrates show representative experiment with 5 WT and 8 CKO mice. Similar results were obtained from 2 independent experiments summarized in table (lower). G-H, Cells were isolated from draining lymph node of mice (d23 post immunization) and co-cultured with increasing dose of MOG₃₅₋₅₅ peptide for 3 days. Antigen-specific cell proliferation is measured by thymidine incorporation (G) and antigen-specific cytokine secretion by legendplex (H). I, Flow cytometry analysis of intracellular transcription factor expression in CD4 T cells isolated from CNS day 15 post immunization. Linear regression analysis (b, c, f, g), two-way anova (e) and student t test (d, i) were used for statistical analysis.

FIG. 38A-38D—Prediction and metabolic validation of the polyamine pathway as a candidate in regulating Th17 cell function. (A) Metabolomics analysis of Th17n (left bar) and Th17p (right bar) cells. Cells were differentiated as described (STAR Methods) and harvested at 68h for LC/MS based metabolomics. Shown are 1,101 differentially expressed metabolites between Th17n and Th17p (BH-adjusted Welch t-test p<0.05), 52 of which are identified and divided between lipids and amino-acid derivatives; (B) Metabolomics analysis of the polyamine pathway as in FIG. 34H. Cell lysates as well as media from Th17n and Th17p differentiation cultures are shown. (C-D) Carbon tracing in the polyamine pathway. Th17n and Th17p cells were differentiated as described (STAR Methods), lifted to rest at 68 hours and pulsed with C13 labeled Arginine (C) or Citrulline (D) followed by LC/MS analysis at time points indicated.

FIG. 39A-39E—Chemical and genetic interference with the polyamine pathway suppress canonical Th17 cell cytokines. (A) The effect of DFMO on cellular polyamine concentration is measured by an enzymatic assay. Th17p, Th17n and iTregs are differentiated in the presence of DFMO and harvested at 96 hours for analysis. (B) Additional analysis of cytokines in supernatant as in FIG. 35C. (C) Protein and phospho-protein analysis by flow cytometry for Th17n and Th17p cells treated with control of DFMO. (D) The effect of DFMO on enzymes in the polyamine pathway as measured by qPCR. Th17p and Th17n cells were differentiated in the presence of control or DFMO and harvested at 48h for RNA extraction and qPCR analysis. (E) The effect of genetic perturbation of ODC1 on cytokine production from Th17p (upper panels) and Th17n cells (lower panels). Supernatant from Th17p and Th17n differentiation culture was harvested at 96 hours and analyzed by legendplex for cytokine concentration.

FIG. 40A-40C—DFMO treatment promotes Treg-like transcriptome and epigenome. (A) Volcano plots showing affected chromatin modifiers by DFMO treatment in Th17n, Th17p and iTreg cells. (B) Number of differentially expressed (DE) peaks between DFMO and vehicle-treated cells as a function of the significance threshold. Upper panel, log 2FC used as threshold; Lower panel, BH-adjusted P used as threshold. (C) Motif enrichment analysis of in vitro differentiated Th17n in the presence or absence of DFMO for Th17 specific genes.

FIG. 41A-41B—Targeting ODC1 and SAT1 alleviate EAE. Cells were isolated from CNS or inguinal lymph node of WT or SAT 1^{fl/fl}CD4^{cre} mice on day 15 post EAE induction (similar experiments as in FIG. 37F). (A) Intracellular cytokines were measured by flow cytometry after 4-hour PMA/ionomycine stimulation ex vivo in the presence of brefaldin and monensin. (B) Transcription factors were analyzed directly ex vivo by intracellular staining.

FIG. 42A-42C—Algorithm overview. (A) Computation of Compass scores matrix. Compass leverages prior knowledge on metabolic topology and stoichiometry (encoded in a GSMM, see main text) to analyze single-cell RNA-Seq expression. Briefly, it computes a reaction-penalties matrix, where the penalty of a given reaction is inversely proportional to the expression its respective enzyme-coding genes. The reaction-penalties matrix is the input to a set of flux-balance linear programs that produce a score for every reaction in every cell, namely the Compass score matrix. (B) To compute the reaction penalties matrix, Compass allows soft information sharing between a cell and its k-nearest neighbors to mitigate technical noise in single-cell library preparation. (C) Downstream analysis of the score matrix. Rows are hierarchically clustered into meta-reactions (agnostically of canonical pathway definitions). The scores are then amenable to common genomics procedures including differential expression of meta-reactions, detecting meta-reactions correlating with a phenotype of interest, dimensionality reduction, and data-driven network analysis (Wang et al., 2020 and Example 8).

FIG. 43A-43E—Compass-based exploration of metabolic heterogeneity within the Th17 compartment. (A) The experimental system. Naive CD4+ T cells are collected and differentiated into Th17p or Th17n cells, which are IL-17+ T cells that cause severe or mild-to-none CNS autoimmunity upon adoptive transfer. Th17nu cells are Th17n cells which were not sorted by IL-17 and exhibit higher variability (Gaublomme et al. 2015). (B) PCA of the Compass scores matrix (restricted to core metabolism, see main text), with select top loadings shown. (C) Dots represent a single biochemical reactions, Cohen's d and Wilcoxon rank sum p values computed as described in the main text for a comparison of Th17p vs. Th17n. This computation is done over meta-reactions, and every meta-reaction is expanded into its constituent single reactions (STAR Methods), each shown as a separate dot. Only core reactions (STAR Methods) are shown. Reactions are partitioned by Recon2 pathways; bottom-right panel groups together all Recon2 subsystems associated with amino-acid metabolism. (D) Spearman correlation of Compass scores of single reactions with the expression of pro-pathogenic (magenta) or pro-regulatory Th17n (green) genes (Gaublomme et al. 2015), none of which is metabolic and therefore none of them directly serves as a Compass input. Only significant correlations (BH-adjusted p<0.1) are shown in color and non-significant correlation coefficients are greyed out. The rows represent 489 meta-reactions that belong to core pathways (defined as Recon2 subsystems that have at least 3 core reactions), and significantly correlated (or anti-correlated) with at least one of the genes. Key reactions (rows) in pathways discussed in the manuscript are highlighted according to the meta-reaction to which they belong. (E) Dots represent single biochemical reactions, Cohen's d and Wilcoxon rank sum p values computed as described in panel C. Only core reactions are shown. Reactions are partitioned by Recon2 pathways. Reactions are divided with a dashed line by the sign of their Cohen d's statistic, and are opaque or transparent according to statistical significance.

FIG. 44A-44I—Differential usage of glycolysis and fatty acid oxidation by pathogenic and non-pathogenic Th17 cells. (A) A diagram of central carbon metabolism, overlaid with Compass prediction for differential potential activity between Th17p and Th17n. Differentially active reactions (BH-adjusted Wilcoxon p<0.1) are shaded for (pro-Th17p) or (pro-Th17n), non-significantly different reactions are also shaded. (B) Th17n, Th17p and Treg cells were differentiated as described (STAR Methods) and replated with Seahorse media at 68h for Seahorse assay. Extracellular acidification rate (ECAR) and oxygen consumption rate (OCR) are reported in response to mitostress test. (C) Th17p and Th17n cells were differentiated and harvested at 68h (left columns) or replated in fresh media with no TCR stimulation or cytokine for 15 minutes (right columns) and subject to LC/MS based metabolomics. (D) Cells were harvested as in C and pulsed with 13C-tagged glucose for 15 minutes. Shown is the ratio of 13C-tagged carbon out of the total carbon content associated with the metabolite (STAR Methods). (E) Th17n and Th17p cells were measured for their oxygen consumption rate in the presence of control or 40 uM etomoxir. (F) Th17n and Th17p cells from either WT or PDK4-deficient mice were differentiated as described (STAR Methods) and replated with Seahorse media at 68h for Seahorse assay. Extracellular acidification rate (ECAR) is reported in response to mitostress test. (G) Number of differentially expressed (DE) genes between PDK4-deficient and WT cells as a function of the significance threshold. (H-I) WT and PDK4−/− mice were immunized with MOG35-55 to induce EAE. (H) EAE clinical score was followed for 21 days. (I) Cells were harvested from CNS at day 15 post immunization for intracellular cytokine or transcription factor analysis.

FIG. 45A-45G—An unexpected role for PGAM in mediating TGFb-induced Th17 pathogenicity. (A) Intra-population analysis in two biological replicates (the Th17n and Th17nu cell populations, see FIG. 43a). Dots are single metabolic reactions, and axes denote their correlation with the pathogenic signature in the Th17nu and Th17n groups. Shading denotes whether the reaction was decided as pro-inflammatory, pro-regulatory, or non-significantly (NS) associated with either state by the inter-population analysis. PGAM, GK, PKM, and G6PD are reactions discussed in the manuscript (see FIG. 45b). SPT=serine-pyruvate transaminase (EC 2.6.1.51). Rev=reverse (backwards) direction. (STAR Methods). (B) Schematics of central carbon metabolism, the highlighted reactions are the two predicted to be most correlated and anti-correlated with the computational pathogenicity score within the Th17n compartment, respectively. Reported inhibitors of these reactions are denoted. (C) Effects of inhibiting candidate genes on Th17 cytokines as measured by flow cytometry are shown. Naive T cells were differentiated under pathogenic (Th17p) and non-pathogenic (Th17n) Th17 cell conditions (STAR Methods) in the presence of control solvent or inhibitors. Cells were pre-labeled with division dye and protein expression is reported for cells that have gone through one division (dl) to exclude arrested cells. (D) PCA of bulk RNA-Seq of dl Th17 cells as in B. (E) Differential gene expression due to EGCG and DHEA treatment. Dots represent genes associated with the pro-pathogenic and pro-regulatory Th17 transcriptional programs, respectively (shaded genes are ones belonging either to the list of pro-pathogenic Th17 markers (FIG. 43d and STAR Methods) or to the Th17 pro-inflammatory covariation module defined by (Gaublomme et al. 2015)). Highly differential genes associated with surface receptors, cytokine activity, or that are otherwise of interest are labelled by name. (F) Histograms of the log FC per gene in differential expression of EGCG- vs. DMSO-treated cells. A separate histogram is shown for Th17p-associated, Th17n-associated, and non-significantly associated genes. Genes were partitioned into these three groups by differential expression in bulk RNA-Seq (same libraries as shown in panel D) between DMSO-treated Th17p and Th17n cells with significance threshold of BH-adjusted p<0.05 and log 2 fold-change ≥1.5 in absolute value. (G) ratio of 13C-tagged carbon to total carbon in Th17 cells cultured for 15 minutes in the presence of 13C-glucose. Three metabolites are shown: PGAM's substrate (3-phosphoglycerate), product (2-phosphoglycerate), and the next downstream metabolite along the glycolytic pathway (phosphoenolpyruvate). (H) Th17n cells were differentiated in the presence of solvent alone, EGCG, PHDGH inhibitor (PKUMDL-WQ-2101, STAR Methods), or the combination. Cells were harvested at 961 for flow cytometric analysis.

FIG. 46A-46J—EGCG exacerbates and DHEA ameliorates Th17-induced EAE in vivo. 2D2 TCR-transgenic Th17 cells were adoptively transferred after differentiation in vitro in the presence of an inhibitor or vehicle as indicated. (A, C, G) Clinical outcome of EAE; (B, D) Histological score based on cell infiltrates in meninges and parenchyma of CNS; (E, F) Draining lymph node (cervical) from respective mice were isolated and pulsed with increasing dose of MOG₃₅₋₅₅ peptide for 3 days and (E) subjected to thymidine incorporation assay; or (F) measurement of cytokine secretion by Legendplex and flow cytometry. Concentrations were normalized through division by the respective response to no antigen control (H-I) Independent pathological report of CNS isolated from mice with EAE at end point (d35 for EGCG experiments; d28 for DHEA experiment); Optic nerves were not found in the histologic section from one animal in the EGCG+IL-23 group. (J) Representative histology of spinal cord and spinal nerve roots. There is greater meningeal inflammation and Wallerian degeneration (digestion chambers, arrows) in posterior spinal nerve roots in EGCG vs. Control mice. PC, posterior column; PH, posterior horn. Individual mouse numbers are indicated. The smaller panel shows VK 39875 mouse section at higher magnification. All are H. & E., 40× objective. Three similar experiments were performed.

FIG. 47—Cumulative distribution function (CDF) of number of reactions per meta-reaction.

FIG. 48A-48H—(A-E) PCA of Compass space restricted to core meta-reactions, see main text. (A) PC1 scores plotted against PC2 and PC3 scores. (B) Enrichment of metabolic pathways in the positive or negative directions of top principal components. Enrichment is computed with GSEA (Subramanian et al. 2005) over single reactions (rather than genes, as in the common applications). Shaded boxes are −log 10(BH-adjusted p), truncated at 4, with p being the GSEA p value. Pathways correspond to Recon2 subsystems. (C) PC1 scores plotted against computational signatures of cellular metabolic activity and Th17 differentiation time course (STAR Methods). (D) Spearman correlation of top PCs with known pro-pathogenic and pro-regulatory marker genes, none of which is metabolic. Only significant correlations (BH-adjusted p<0.1) are shown shaded. (E) Spearman correlation of computational transcriptome signatures with the top principal components. Only significant correlations (BH-adjusted p<0.1) are shown shaded and non-significant correlation coefficients are greyed out. See STAR Methods for signature computation. (F) Same analysis as shown in FIG. 2c, but showing all reactions (and not just ones belonging to certain pathways, as in the main figure). (G) Applicants computed a pro-pathogenic score for each reaction by taking the ratio of pro-pathogenic and pro-regulatory markers with which it correlates and anti-correlates, respectively (BH-adjusted p<0.1 for a Spearman correlation) out of the 23 marker genes (listed in FIG. 43d and STAR Methods). Similarly, Applicants computed pro-regulatory reaction scores. Only core reactions are shown. (H) Same analysis as shown in FIG. 43E, only at the gene expression level (and not reaction level based on Compass scores). Genes are grouped by KEGG pathways (and may be annotated as belonging to more than one pathway).

FIG. 49A-49F—(A) Parallel of main FIG. 44c showing also 44h after fresh media pulse. (B) The glycolysis pathway, as shown in main FIG. 45a, highlighting PDH and associated reactions. (C) PDK4 transcript expression in the experiment described in main FIG. 45c. (D) Dots are transcriptomic computational signatures (STAR Methods), axes correspond to the fold-change in the signature's value in comparisons of PDK4-deficient cells vs. WT cells in Th17p (x-axis) and Th17n (y-axis). (E) Th17 cells from PDK4−/− and WT mice were subject to LC/MS metabolomics as in main FIG. 44c, having been replated for 15 minutes. (F) metabolites associated with amino-acid metabolic pathways in the assay described in main FIG. 44c.

FIG. 50A-50D—(A) Same data as shown in FIG. 45a, highlighting the reactions with significant adjusted Fisher p value in the intra-population analysis; every reaction is assigned a combined Fisher p-value of the two p-values measuring the significance of the correlation with the two axes (STAR Methods). Search space was limited to core reactions. (B-C) Hypergeometric enrichment of the targets identified by the inter-population analysis (reactions with differential potential activity between Th17p and Th17n, decided by a BH-adjusted p cutoff) in targets identified by the intra-population analysis (reactions identified by a BH-adjusted Fisher p cutoff) while varying the cutoffs. (D) Supernatant from Th17 cell cultures performed for main FIG. 45c are harvested for cytokine analysis using Legendplex.

FIG. 51A-51B—Cytokine secretion after three days of culture with increasing dose of MOG35-55 peptide from cells isolated from draining lymph node (cervical) of mice transferred with (A) methanol or DHEA treated Th17p cells as in FIG. 46A or (B) DMSO or EGCG as in FIG. 46C. Concentrations were normalized through division by the respective response to no antigen control.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +1-5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to the manuscript and included tables submitted in a Biorxiv posting titled “Metabolic and Epigenomic Regulation of Th17/Treg Balance by the Polyamine Pathway,” by Chao Wang, Allon Wagner, Johannes Fessler, Julian Avila-Pacheco, Jim Karminski, Pratiksha Thakore, Sarah Zaghouani, Kerry Pierce, Lloyd Bod, Alexandra Schnell, David DeTomaso, Noga Ron-Harel, Marcia Haigis, Daniel Puleston, Erika Pearce, Manoocher Soleimani, Ray Sobel, Clary Clish, Aviv Regev, Nir Yosef, and Vijay K. Kuchroo, (Wang et al., 2020). Reference is also made to the manuscript and included Tables submitted in a Biorxiv posting titled “In Silico Modeling of Metabolic State in Single Th17 Cells Reveals Novel Regulators of Inflammation and Autoimmunity,” by Allon Wagner, Chao Wang, David DeTomaso, Julian Avila-Pacheco, Sarah Zaghouani, Johannes Fessler, Elliot Akama-Garren, Kerry Pierce, Noga Ron-Harel, Vivian Paraskevi Douglas, Marcia Haigis, Raymond A. Sobel, Clary Clish, Aviv Regev, Vijay K. Kuchroo, and Nir Yosef, (Wagner et al., 2020).

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide methods of shifting T cell balance in a population of cells comprising T cells and therapeutic compositions thereof. Embodiments disclosed herein also provide for methods of treating inflammatory diseases and autoimmune responses. In certain embodiments, T cell differentiation is shifted towards or away from Th17 cell gene expression, is shifted towards or away from T_{reg} cell gene expression, and/or is shifted towards or away from Th1 cell gene expression. In certain embodiments, T cell balance is shifted by contacting the T cells with a polyamine, polyamine analogue or an agent capable of modulating the polyamine pathway. In certain embodiments, T cell balance is shifted by contacting the T cells with a drug targeting a reaction in the glycolysis pathway.

Cellular metabolism is a powerful regulator of immune responses. Th17 cells become very proliferative and active after they are stimulated by an antigen, and this transition depends on a metabolic shift—from oxidative phosphorylation to glycolysis. This shift makes them divergent from immunosuppressive T cells that remain dependent on fatty acid oxidation and the TCA cycle (see, e.g., O'Sullivan & L Pearce, 2014, Fatty acid synthesis tips the TH17-Treg cell balance, Nature Medicine volume 20, pages 1235-1236). For example, Tregs are dependent on fatty acid oxidation and oxidative phosphorylation and Th17 cells are dependent on de-novo fatty acid synthesis and glycolysis.

Applicants identified the polyamine pathway and glycolysis pathway as associated with Th17 cell pathogenicity using both novel algorithm (COMPASS) and fluxomics/metabolomics. Applicants analyzed metabolic pathways associated with Th17 pathogenicity using COMPASS, a computational algorithm used to characterize the metabolic landscape of single cells based on single-cell RNA-Seq profiles and flux balance analysis. Applicants used COMPASS to characterize the metabolic heterogeneity in Th17 cells, whose pathogenic state triggers auto-immunity, yet whose non-pathogenic form promotes tissue homeostasis and barrier functions. COMPASS recovered known metabolic switches and predicted that the polyamine pathway should be a novel, powerful regulator of Th17 pathogenicity. Applicants validated the pathway's effect through an array of transcriptome, LC/MS metabolome, and functional assays. Deletion of polyamine enzymes in T cells resulted in altered metabolic space, T cell functions and, most importantly, aggravated symptoms in EAE, a murine model of multiple sclerosis. Further, Applicants identified for the first time that treatment of T cells with polyamines and polyamine analogues altered T cell balance. Applicants showed that inhibition of the polyamine pathway by a drug, DFMO, in Th17 cells are effective in reducing canonical Th17 genes and shift Th17 cells to Treg-like transcriptome. DFMO specifically reduced accessibility in regions specific to Th17 cells in ATAC-seq. Applicants showed that DFMO is effective in reducing EAE. DFMO reduces the expression of the enzyme Sat1, an enzyme involved in the polyamine pathway and Applicants showed conditional deletion of Sat1 in T cells resulted in increased Treg frequency, delayed EAE onset and reduced severity similar to DFMO treatment. Applicants also identified that polyamines are significantly upregulated in MS patients and in IBD patients. Applicants identified inhibitors of glycolysis pathway enzymes could also shift Th17 pathogenicity.

Cellular metabolism can orchestrate immune cell function. Previously it was demonstrated that lipid biosynthesis represents one such gatekeeper to Th17 cell functional state. Utilizing a transcriptome-based in silico fluxomics tool, Applicants constructed a comprehensive metabolic circuitry in association with Th17 cell function and identified the polyamine pathway as a candidate metabolic node, the flux of which regulates the inflammatory function of T cells. Indeed, expression and activities of enzymes of the polyamine pathway are suppressed in regulatory T cells and Th17 cells at the regulatory state. Perturbation of the polyamine pathway in Th17 cells suppressed canonical Th17 cell cytokines and promoted the expression of Foxp3, accompanied by dramatic shift in transcriptome and epigenome, transition Th17 cells into a Treg-like state in a cMaf dependent manner. Importantly, genetic and molecular perturbation of the polyamine pathway resulted in attenuation of autoimmune inflammation in the EAE model.

Cellular metabolism is a powerful modulator of immune response that can now be studied through the lens of single-cell RNA-Seq. However, single-cell analysis requires novel computational methods to address its unique challenges and unlock its unique potential. Here, Applicants present Compass, an algorithm to characterize the metabolic landscape of single cells based on single-cell RNA-Seq profiles and flux balance analysis. Applicants used Compass to study the landscape of metabolic heterogeneity in T helper 17 (Th17) cells and search for novel metabolic regulators of their immune functions. Compass recovered known metabolic switches but surprisingly predicted a glycolytic reaction (phosphoglycerate mutase) that, contrary to common immunometabolic understanding of glycolysis, promotes an anti-inflammatory phenotype. Applicants validated the predicted effects through an array of transcriptome, LC/MS and 13C-traced metabolome, and functional assays. While the study is concerned with Th17 cells, Compass is generally applicable, and can be used to characterize the metabolic states of any cell population based on its single-cell transcriptome profiles.

### Methods of Shifting T Cell Balance

### T Cells

T lymphocytes include a variety of T cell types, e.g., Th17, regulatory T cells (Tregs), Treg-like cells, Th1 cells or Th1-like cells, or naïve T cells. As used herein, terms such as “Th17 cell” and/or “Th17 phenotype” and all grammatical variations thereof refer to a differentiated T helper cell that expresses one or more cytokines selected from the group the consisting of interleukin 17A (IL-17A), interleukin 17F (IL-17F), and interleukin 17A/F heterodimer (IL17-AF). As used herein, terms such as “Th1 cell” and/or “Th1 phenotype” and all grammatical variations thereof refer to a differentiated T helper cell that expresses interferon gamma (IFNγ). As used herein, terms such as “Th2 cell” and/or “Th2 phenotype” and all grammatical variations thereof refer to a differentiated T helper cell that expresses one or more cytokines selected from the group the consisting of interleukin 4 (IL-4), interleukin 5 (IL-5) and interleukin 13 (IL-13). As used herein, terms such as “Treg cell” and/or “Treg phenotype” and all grammatical variations thereof refer to a differentiated T cell that expresses Foxp3. “Naive T cells” and/or “naïve T cell phenotype” and all grammatical variations thereof as used herein are typically unable to produce proinflammatory cytokines, and are precursors for T-effector subsets. Naive T cells typically lack expression of previous activation, such as, for example, CD25, CD44, CD69, CD45RO, or HLA-DR. (see, e.g. T. Eagar and S. Miller, 2019, Helper T-Cell Subsets and Control of the Inflammatory Response, Clinical Immunology (Fifth Edition), 2019).

### Th17, Treg, Th1 T Cell Balance

The invention also provides compositions and methods for modulating T cell balance. The invention provides T cell modulating agents that modulate T cell balance. For example, in some embodiments, the invention provides T cell modulating agents and methods of using these T cell modulating agents to regulate, influence, shift or otherwise impact the level of and/or balance between T cell types, e.g., between Th17 and other T cell types, for example, regulatory T cells (Tregs), Treg-like cells, Th1 cells or Th1-like cells. For example, in some embodiments, the invention provides T cell modulating agents and methods of using these T cell modulating agents to regulate, influence, shift, or otherwise impact the level of and/or balance between Th17 activity and inflammatory potential. Shifting the balance in a population of cells comprising T cells can comprise a change in T cell differentiation. T cell differentiation can shift towards non-pathogenic Th17 cells, Th1 cells, Treg cells, and/or is shifted away from pathogenic Th17 cells, Treg cells, or Th1 cells. Methods shifting the T cell balance can comprise differentiation of naïve T cells into Th17 cells, Th1 cells and/or Treg cells.

As used herein, terms such as “pathogenic Th17 cell” and/or “pathogenic Th17 phenotype” and all grammatical variations thereof refer to Th17 cells that, when induced in the presence of TGF-β3 or TGF-β1+IL-6+IL-23, express an elevated level of one or more genes selected from Cxcl3, IL22, IL3, Cc14, Gzmb, Lrmp, Ccl5, Casp1, Csf2, Ccl3, Tbx21, Icos, IL17r, Stat4, Lgals3 and Lag, as compared to the level of expression in TGF-β1+IL-6-induced Th17 cells. As used herein, terms such as “non-pathogenic Th17 cell” and/or “non-pathogenic Th17 phenotype” and all grammatical variations thereof refer to Th17 cells that, when induced in the presence of TGF-β1+IL-6, express an increased level of one or more genes selected from IL6st, IL1rn, Ikzf3, Maf, Ahr, IL9 and IL10, as compared to the level of expression in a TGF-β3-induced or TGF-β1+IL-6+IL-23-induced Th17 cells.

Depending on the cytokines used for differentiation (pathogenic conditions are TGF-β3 or TGF-β1+IL-6+IL-23 and non-pathogenic conditions are TGF-β1+IL-6), in vitro polarized Th17 cells can either cause severe autoimmune responses upon adoptive transfer (‘pathogenic Th17 cells’) or have little or no effect in inducing autoimmune disease (‘non-pathogenic cells’) (Ghoreschi et al., 2010; Lee et al., 2012). In vitro differentiation of naïve CD4 T cells in the presence of TGF-β1+IL-6 induces an IL-17A and IL-10 producing population of Th17 cells, that are generally nonpathogenic, whereas activation of naïve T cells in the presence IL-1β+IL-6+IL-23 induces a T cell population that produces IL-17A and IFN-γ, and are potent inducers of autoimmune disease induction (Ghoreschi et al., 2010).

A dynamic regulatory network controls Th17 differentiation (See e.g., Yosef et al., Dynamic regulatory network controlling Th17 cell differentiation, Nature, vol. 496: 461-468 (2013); Wang et al., CD5L/AIM Regulates Lipid Biosynthesis and Restrains Th17 Cell Pathogenicity, Cell Volume 163, Issue 6, p1413-1427, 3 Dec. 2015; Gaublomme et al., Single-Cell Genomics Unveils Critical Regulators of Th17 Cell Pathogenicity, Cell Volume 163, Issue 6, p1400-1412, 3 Dec. 2015; and International Patent Publication Nos. WO2016138488A2, WO2015130968, WO/2012/048265, WO/2014/145631 and WO/2014/134351 the contents of which are hereby incorporated herein by reference in their entirety). Accordingly, shifting the T cell balance in a population of cells may include contacting the population of cells with IL-6 and TGF-β1 or IL-1β, IL-6, and IL-23. In certain embodiments, the IL-6 and TGF-β1 or IL-1β, IL-6, and IL-23 supplement a cell culture media. In one embodiment, the administration of the agents differentiates naïve T cells into Th17 cells. Optionally, the agents are administered to the population of cells during differentiation.

### Contacting a Population of Cells

As used herein, a population of cells contacted with one or more agents can be in vivo or in vitro or ex vivo.

### Polyamines and the Polyamine Pathway

As used herein, the term “polyamine” refers to an organic compound having more than two amino groups. Polyamines are naturally occurring polycations that are required for cell growth, and manipulation of cellular polyamine levels can lead to decreased proliferation, and, in some cases, increased cell death. Natural polyamine biosynthesis is regulated by the rate-limiting enzymes ornithine decarboxylase (ODC) and S-Adenosylmethionine decarboxylase (SAMDC), while polyamine catabolism is driven by spermidine/spermine N¹-acetyltransferase/polyamine oxidase (SSAT/PAO) and spermine oxidase SMO(PAOh1). (See, e.g., Huang et al., Cancer Biol Ther. 2005 September; 4(9): 1006-1013).

In certain embodiments, genes and polypeptides belonging to the polyamine pathway are modulated or targeted. All gene name symbols as used herein refer to the gene as commonly known in the art. The examples described herein that refer to the mouse gene names are to be understood to also encompasses human genes, as well as genes in any other organism (e.g., homologous, orthologous genes). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). Any reference to the gene symbol is a reference made to the entire gene or variants of the gene. The signature as described herein may encompass any of the genes described herein.

The gene name SAT1, SSAT-1, SSAT, SAT, Spermidine/Spermine N1-Acetyltransferase 1, Polyamine N-Acetyltransferase 1, Diamine N-Acetyltransferase 1, Putrescine Acetyltransferase, Spermidine/Spermine N1-Acetyltransferase Alpha, Spermidine/Spermine N(1)-Acetyltransferase 1, Spermidine/Spermine N1-Acetyltransferase, Diamine Acetyltransferase 1, EC 2.3.1.57, KF SDX, DC21, and KFSD may refer to the gene or polypeptide according to NCBI Reference Sequence accession numbers NM_002970.3 and NM_009121.4. SAT1 is a highly regulated enzyme that allows a fine attenuation of the intracellular concentration of polyamines. SAT1 is also involved in the regulation of polyamine transport out of cells. SAT1 acts on 1,3-diaminopropane, 1,5-diaminopentane, putrescine, spermidine (forming N(1)- and N(8)-acetyl spermidine), spermine, N(1)-acetyl spermidine and N(8)-acetyl spermidine. As described further herein, SAT1 is a top-ranking gene associated with Th17 pathogenicity and SAT1 activity is associated with pathogenicity.

### Modulating Agents

As used herein, “modulating” or “to modulate” generally means either reducing or inhibiting the expression or activity of, or alternatively increasing the expression or activity of a target (e.g., polyamine pathway). In particular, “modulating” or “to modulate” can mean either reducing or inhibiting the activity of, or alternatively increasing a (relevant or intended) biological activity of, a target or antigen as measured using a suitable in vitro, cellular or in vivo assay (which will usually depend on the target involved), by at least 5%, at least 10%, at least 25%, at least 50%, at least 60%, at least 70%, at least 80%, at least 90%, or more, compared to activity of the target in the same assay under the same conditions but without the presence of an agent. An “increase” or “decrease” refers to a statistically significant increase or decrease respectively. For the avoidance of doubt, an increase or decrease will be at least 10% relative to a reference, such as at least 10%, at least 20%, at least 30%, at least 40%, at least 50%, at least 60%, at least 70%, at least 80%, at least 90%, at least 95%, at least 97%, at least 98%, or more, up to and including at least 100% or more, in the case of an increase, for example, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more. “Modulating” can also involve effecting a change (which can either be an increase or a decrease) in affinity, avidity, specificity and/or selectivity of a target or antigen, such as polyamine pathway enzyme binding. “Modulating” can also mean effecting a change with respect to one or more biological or physiological mechanisms, effects, responses, functions, pathways or activities in which the target or antigen (or in which its substrate(s), ligand(s) or pathway(s) are involved, such as its signaling pathway or metabolic pathway and their associated biological or physiological effects) is involved. Again, as will be clear to the skilled person, such an action as an agonist or an antagonist can be determined in any suitable manner and/or using any suitable assay known or described herein (e.g., in vitro or cellular assay), depending on the target or antigen involved.

Modulating can, for example, also involve allosteric modulation of the target and/or reducing or inhibiting the binding of the target to one of its substrates or ligands and/or competing with a natural ligand, substrate for binding to the target. Modulating can also involve activating the target or the mechanism or pathway in which it is involved. Modulating can for example also involve effecting a change in respect of the folding or confirmation of the target, or in respect of the ability of the target to fold, to change its conformation (for example, upon binding of a ligand), to associate with other (sub)units, or to disassociate. Modulating can for example also involve effecting a change in the ability of the target to signal, phosphorylate, dephosphorylate, and the like.

### Polyamine Analogues

In certain embodiments, a T cell modulating agent comprises a polyamine analogue. Polyamine analogues have been synthesized as metabolic modulators that deplete natural intracellular polyamine pools, or polyamine mimetics that displace the natural polyamines from binding sites, but do not substitute for their growth promoting function. Symmetrically substituted bis(alkyl)polyamine analogues represent the first generation of these analogues, some of which downregulate polyamine biosynthesis and increase SSAT activity in certain tumor cell types like non-small cell lung cancer cells, melanoma and human breast cancer cells. A second generation of polyamine analogues are unsymmetrically substituted compounds that display structure-dependent and cell type-specific effects on regulation of polyamine metabolism. Recently, a series of new polyamine analogues designated conformationally restricted, cyclic and oligoamine analogues have been developed. Some of these agents incorporate alterations that limit the free rotation of the single bonds in otherwise flexible molecules such as spermine or its analogues, thus restricting the molecular conformation that they may assume. Oligoamine analogues consist of synthetic octa-, deca-, dodeca- and tetradecamines with longer chains than natural mammalian polyamine molecules, with or without conformational restriction. Some of these novel analogues have shown significant activity against multiple human tumors both in vitro and in vivo (See, e.g., Huang et al., Cancer Biol Ther. 2005 September; 4(9): 1006-1013).

The fluorinated ornithine analog α-difluoromethylornithine (DFMO, eflornithine, alpha-difluoromethylomithine, Ornidyl®, Vaniqa®) is an FDA approved irreversible suicide inhibitor of ornithine decarboxylase (ODC), the first and rate-limiting enzyme of polyamine biosynthesis (see, e.g., LoGiudice et al., Alpha-Difluoromethylornithine, an Irreversible Inhibitor of Polyamine Biosynthesis, as a Therapeutic Strategy against Hyperproliferative and Infectious Diseases. Med. Sci. 2018, 6(1), 12; US20170273926A1). DFMO is a structural analog of the amino acid L-omithine and has a chemical formula C₆H₁₂N₂O₂F₂. DFMO can be employed in the methods of the invention as a racemic (50/50) mixture of D- and L-enantiomers, or as a mixture of D- and L-isomers where the D-isomer is enriched relative to the L-isomer, for example, 70%, 80%, 90% or more by weight of the D-isomer relative to the L-isomer. The DFMO employed may also be substantially free of the L-enantiomer.

The initial promise of DFMO as a therapeutic ODC inhibitor for use in the treatment of various neoplasias has failed to translate into the clinic because, although DFMO does, in fact, irreversibly inhibit ODC activity, cells treated in vivo with DFMO significantly increase their uptake of exogenous putrescine as described in U.S. Pat. No. 4,925,835.

The use of eflornithine (DFMO) is disclosed in U.S. Pat. No. 6,653,351. U.S. Pat. No. 6,277,411 discloses formulations for the administration of eflornithine, including a core having a rapid release DFMO-containing granules and a slow release granule and an outer layer surrounding the core comprising a pH responsive coating.

In certain embodiments, DFMO can be administered either orally or by injection, such as intravenously or intraperitoneally. In certain embodiments, the daily dose of DFMO is about 3.0 to 9.0 g/m2 given in three equal administrations each eight hours. In other embodiments, the dose of eflornithine may be varied considering the treatment and condition of the subject. Such modifications of dosage are generally routine to one of skill in the art. The forms of eflomithine include both isolated L-eflornithine and D-eflornithine, as well as a racemic mixture of L- and D-eflornithine. A higher dose of the D-form may be utilized, such as about 20 g/m2, about 30 g/m2, about 40 g/m2, or about 50 g/m2. Strategies to make DFMO more acceptable to human patients are described in U.S. Pat. No. 4,859,452. Formulations of DFMO are described which include essential amino acids in combination with either arginine or omithine to help reduce DFMO-induced toxicities.

### Histone Demethylation Inhibitors

In certain embodiments, a histone demethylation agent is used to modulate Th17/Treg balance. A non-limiting example inhibitor is GSK-J1 (C₂₂H₂₃N₅O₂) (see, e.g., Kruidenier et al (2012) A selective jumonji H3K27 demethylase inhibitor modulates the proinflammatory macrophage response. Nature 488 404; and Heinemann et al (2014) Inhibition of demethylases by GSK-J1/J4. Nature 514 E1). GSK-J1 is a Potent inhibitor of the H3K27 histone demethylases JMJD3 (KDM6B) and UTX (KDM6A) (IC50 values are 28 and 53 nM respectively). GSK-J1 also inhibits KDMSB, KDMSC and KDMSA (IC50 values are 170, 550 and 6,800 nM respectively). GSK-J1 exhibits no activity against a panel of other histone demethylases (IC50>20 μM), and displays no significant inhibitory activity against 100 protein kinases at a concentration of 30 μM.

### Small Molecules

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810).

In certain embodiments, the small molecule inhibits an enzyme in the polyamine pathway. In certain embodiments, the small molecule includes, but is not limited to diminazene aceturate (Berenil) (PMID: 1510731) (inhibitor of SAT1), trans-4-methyl cyclohexyl amine (MCHA) (spermidine synthase inhibitor), or N-(3-aminopropyl)cyclohexylamine (APCHA) (spermine synthase inhibitor).

In certain embodiments, the small molecule targets an enzyme in the glycolysis pathway. The small molecules may modulate the activity or function of a gene or gene product selected from the group consisting of: PGAM, G6PD, PKM, Aldo, PFKM, TA, G6PC, GK, ENO1, PCK1, TPI1, PGK1, GAPDHS, PDHA1, and GPD1. Small molecules known to inhibit the enzymes include EGCG (see, e.g., Nagle, et al., Epigallocatechin-3-gallate (EGCG): Chemical and biomedical perspectives, Phytochemistry. 2006 September; 67(17): 1849-1855), 2,5-Anhydro-D-glucitol-1,6-diphosphate (see, e.g., US20180133192A1), S-hexadecyl-CoA (S-HD-CoA) (see, e.g., Jenkins et al., Reversible High Affinity Inhibition of Phosphofructokinase-1 by Acyl-CoA-A MECHANISM INTEGRATING GLYCOLYTIC FLUX WITH LIPID METABOLISM, J Biol Chem. 2011 Apr 8; 286(14): 11937-11950), DHEA (see, e.g., Schwartz and Pashko, Dehydroepiandrosterone, glucose-6-phosphate dehydrogenase, and longevity. Ageing Res Rev. 2004 Apr; 3(2):171-87), poldatin (see, e.g., Mele, et al., A new inhibitor of glucose-6-phosphate dehydrogenase blocks pentose phosphate pathway and suppresses malignant proliferation and metastasis in vivo, Cell Death & Disease volume 9, Article number: 572 (2018)), TX1 (see, e.g., Stancu, et al., fasebj.31.1_supplement.921.1; and Cho, et al., A Fluorescence-Based High-Throughput Assay for the Identification of Anticancer Reagents Targeting Fructose-1,6-Bisphosphate Aldolase. SLAS Discov. 2018 January; 23(1):1-10), Gimeracil (see, e.g., Sakata, et al., Gimeracil, an inhibitor of dihydropyrimidine dehydrogenase, inhibits the early step in homologous recombination. Cancer Sci. 2011 September; 102(9):1712-6), Shikonin (see, e.g., Wang, et al., PKM2 Inhibitor Shikonin Overcomes the Cisplatin Resistance in Bladder Cancer by Inducing Necroptosis. Int J Biol Sci. 2018 Oct 20; 14(13):1883-1891), Pyruvate Kinase Inhibitor III (see, e.g., Vander Heiden, M. G., et al. 2010. Biochem. Pharmacol. 79, 1118), 2,3-dihydroxypropyl dichloroacetate (DCA) (see, e.g., Tisdale and Threadgill, (+/−)2,3-Dihydroxypropyl dichloroacetate, an inhibitor of glycerol kinase. Cancer Biochem Biophys. 1984 September; 7(3):253-9), 2,9-Dimethyl-BC (see, e.g., Bonnet, et al., The strong inhibition of triosephosphate isomerase by the natural beta-carbolines may explain their neurotoxic actions. Neuroscience. 2004; 127(2):443-53), Koningic acid (see, e.g., Endo A et al. Specific inhibition of glyceraldehyde-3-phosphate dehydrogenase by koningic acid (heptelidic acid). J Antibiot (Tokyo) 38:920-5 (1985)), CBR-470-1 (see, e.g., Bollong, et al., A metabolite-derived protein modification integrates glycolysis with KEAP1-NRF2 signalling. Nature. 2018 October; 562(7728):600-604), SF2312 (see, e.g., Leonard, et al., SF2312 is a natural phosphonate inhibitor of enolase. Nat Chem Biol. 2016 December; 12(12):1053-1058), PhAh (see, e.g., Anderson, et al., “Reaction intermediate analogues for enolase,” Biochemistry, 23(12):2779-2789, 1984), ENOblock (see, e.g., Cho, et al., ENOblock, a unique small molecule inhibitor of the non-glycolytic functions of enolase, alleviates the symptoms of type 2 diabetes. Sci Rep. 2017 Mar. 8; 7:44186), 3-MPA (see, e.g., Ma, et al. A Pck1-directed glycogen metabolic program regulates formation and maintenance of memory CD8+ T cells. Nat Cell Biol. (2018) 20:21-7), and 6,8-Bis(benzylthio)octanoic acid (see, e.g., U.S. Ser. No. 10/391,177B2). Shikonin inhibits PKM2, dehydroepiandrosterone (DHEA) inhibits G6PD, epigallocatechin-3-gallate (EGCG) inhibits PGAM1, and 2,3-dihydroxypropyl-dichloroacetate (DCA) inhibits GK.

### Genetic Modifying Agents

In certain embodiments, the one or more modulating agents may be a genetic modifying agent. The genetic modifying agent may comprise a CRISPR system, a zinc finger nuclease system, a TALEN, a meganuclease or RNAi system. In some embodiments, a polynucleotide of the present invention described elsewhere herein can be modified using a genetic modifying agent (e.g., one or more target genes are selected from SAT1, ODC1, SRM, SMS, JMJD3, POU2F2, POU2F1, POU5F1B, POU3F4, POU1F1, POU3F2, POU3F3, POU4F2, POU2F3, POU3F1, POU4F1, NFAT5, NFATC2, c-MAF and BATF; or one or more target genes are selected from PGAM, G6PD, PKM, Aldo, PFKM, TA, G6PC, GK, ENO1, PCK1, TPI1, PGK1, GAPDHS, PDHA1, and GPD1; or a combination of one or more of the genes). In preferred embodiments, modulation of expression or a gene using a genetic modifying agent (e.g., enzyme) is temporary (e.g., modulated for a period of time to shift T cell balance without adverse effects). Temporary modulation may be achieved by targeting RNA (e.g., RNA targeting CRISPR system, RNAi) or by targeting regulatory elements (e.g., CRISPRa/i).

### CRISPR-Cas Modification

In some embodiments, a polynucleotide of the present invention described elsewhere herein can be modified using a CRISPR-Cas and/or Cas-based system.

In general, a CRISPR-Cas or CRISPR system as used in herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (transactivating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two class are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

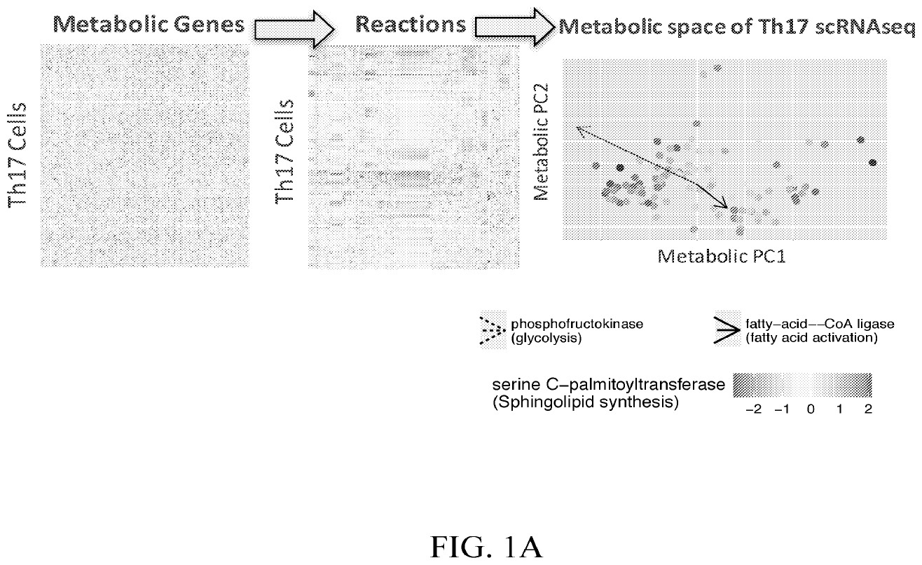

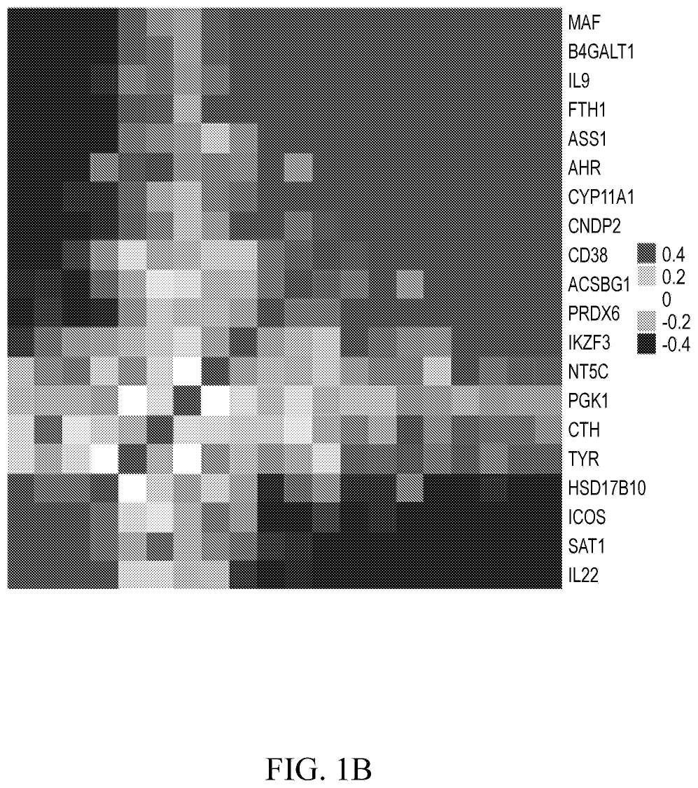

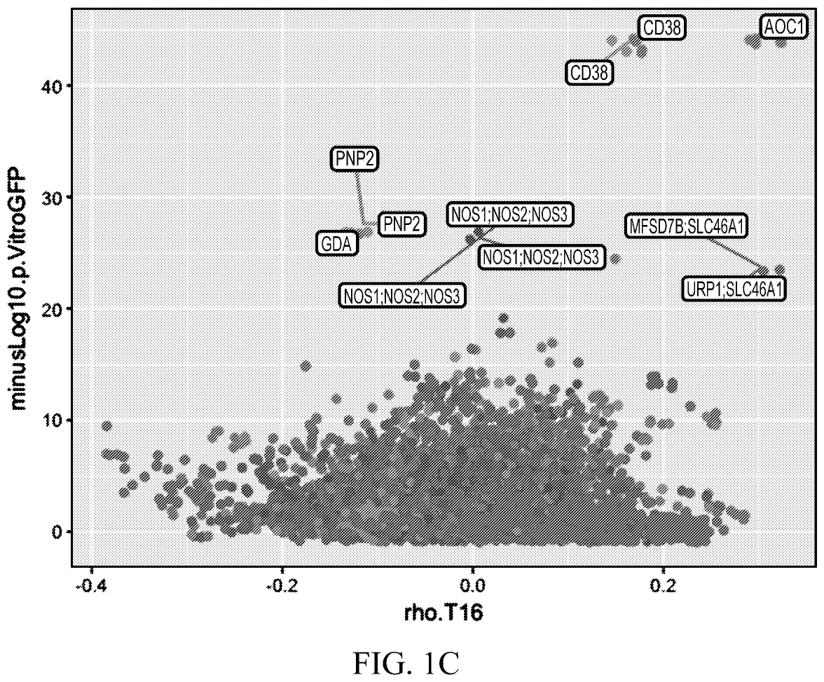

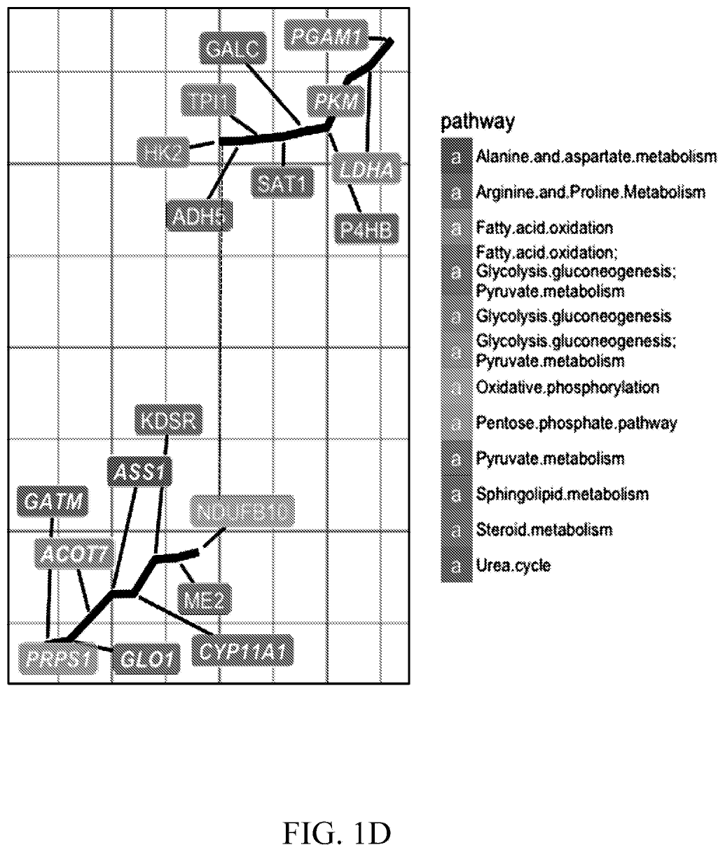

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18: 67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

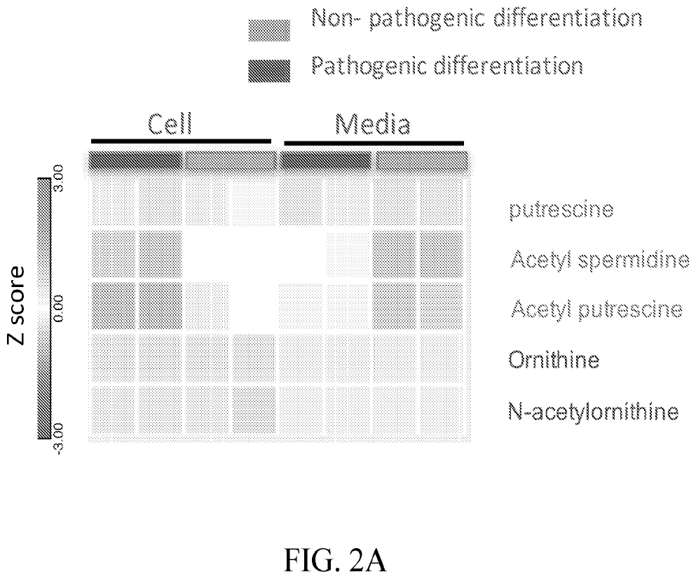

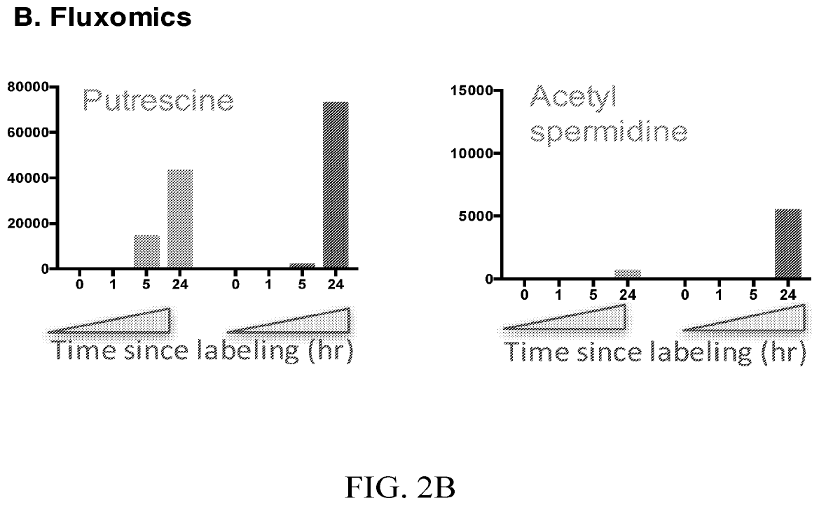

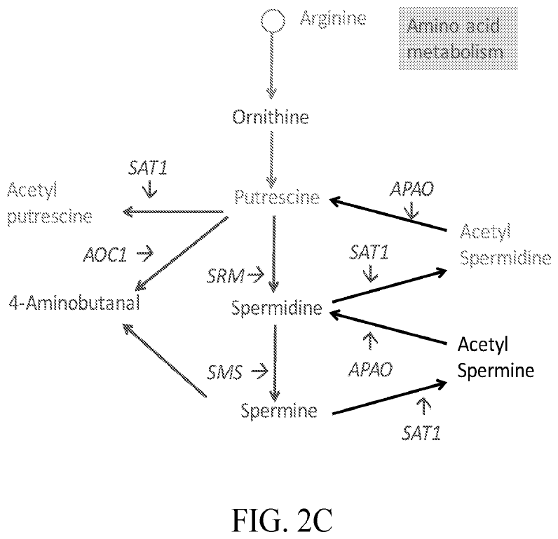

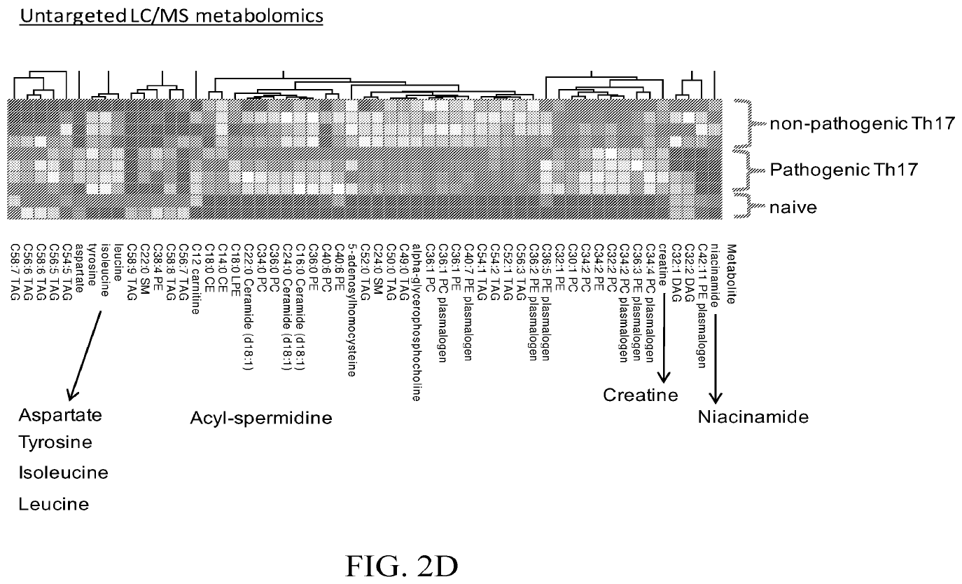

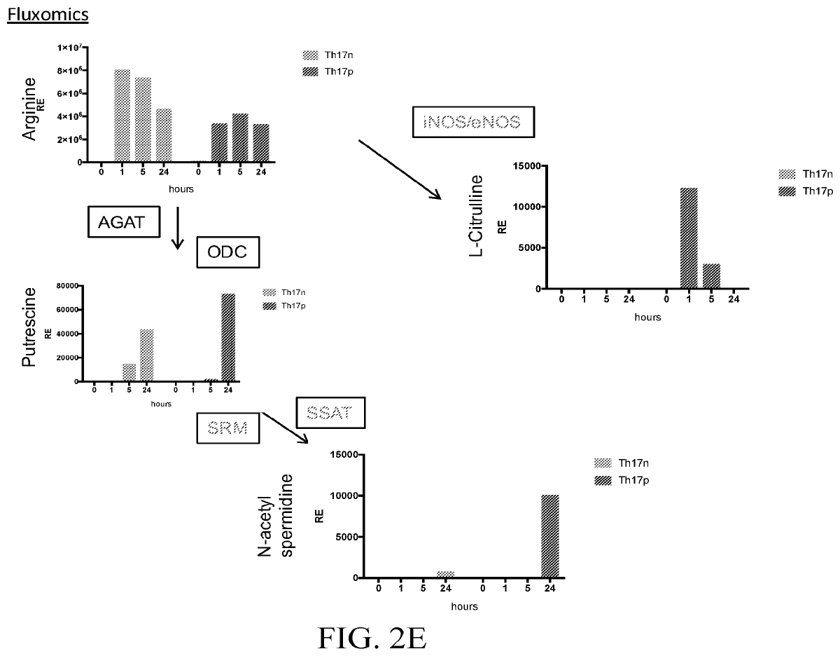

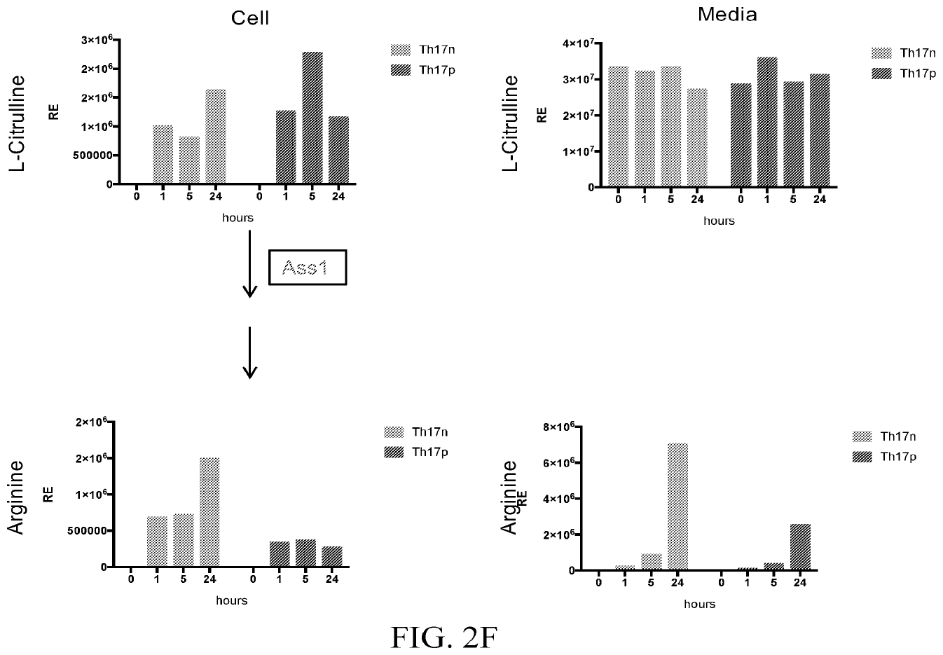

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cash 1). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cash, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasX, and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SETT/9), a translation initiation domain, a transcriptional repression domain (e.g., a KRAB domain, NuE domain, NcoR domain, and a SID domain such as a SID4X domain), a nuclease domain (e.g., Fold), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO 2019/005884, and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Application Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C•G base pair into a T•A base pair (Komor et al. 2016. Nature. 533:420-424; Nishida et al. 2016. Science. 353; and Li et al. Nat. Biotech. 36:324-327) and ABEs convert an A•T base pair to a G•C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708 and WO 2018/213726, and International Patent Application Nos. PCT/US2018/067207, PCT/US2018/067225, and PCT/US2018/067307, which are incorporated herein by reference.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system. See e.g. Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g. sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g. a prime editing guide molecule or peg guide molecule) directly into the target site in the target polynucleotide. See e.g. Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g. is a Cas9 nickase). In some embodiments, the Cas polypeptide is fused to the reverse transcriptase. In some embodiments, the Cas polypeptide is linked to the reverse transcriptase.

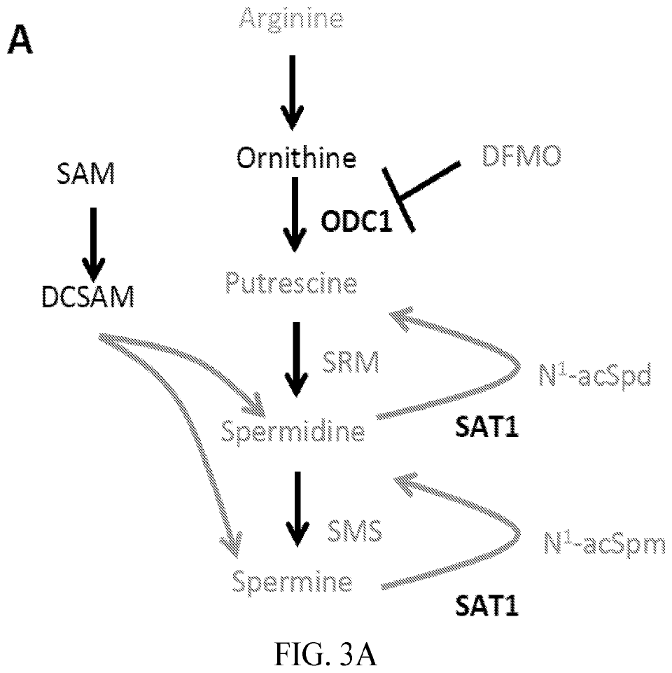

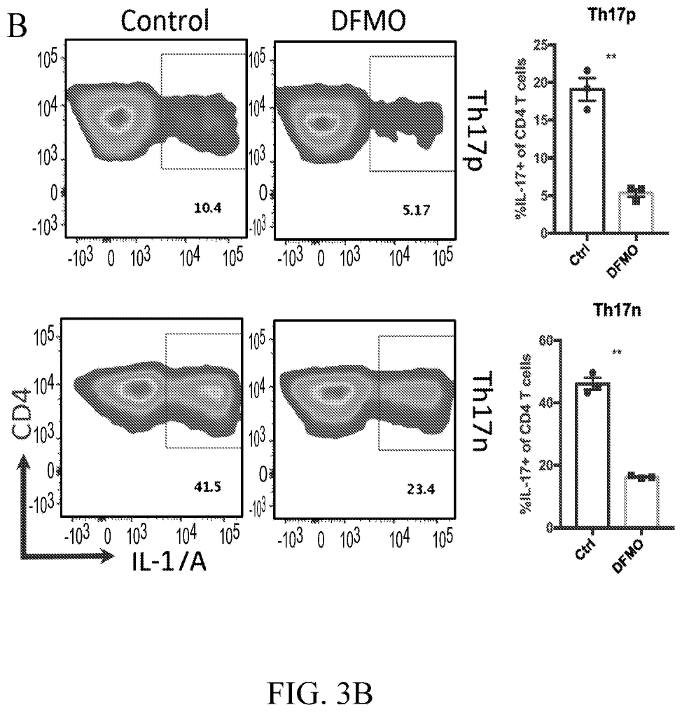

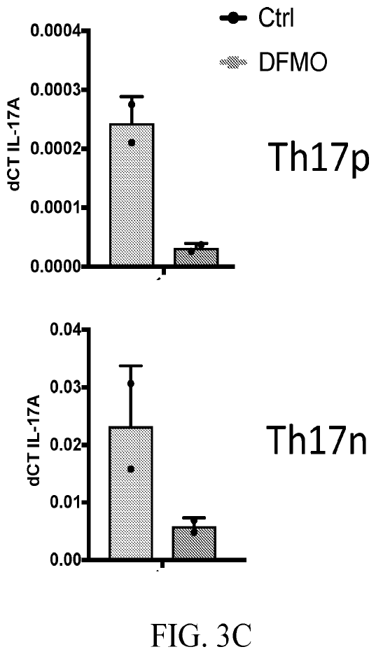

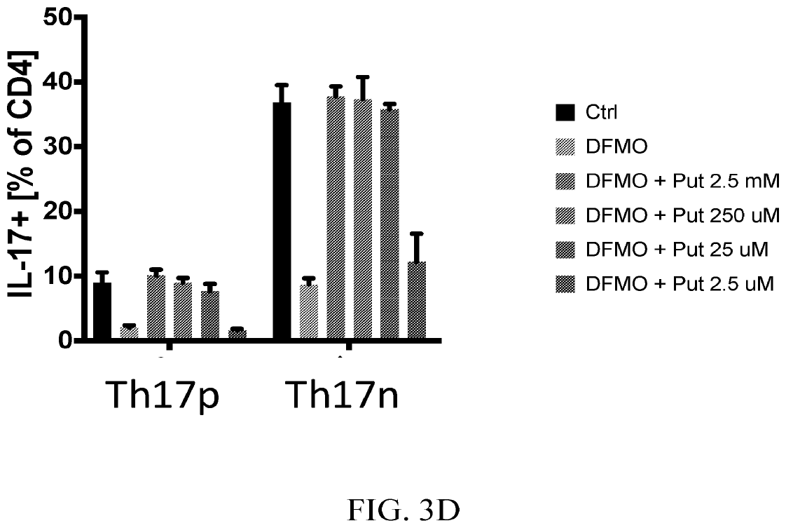

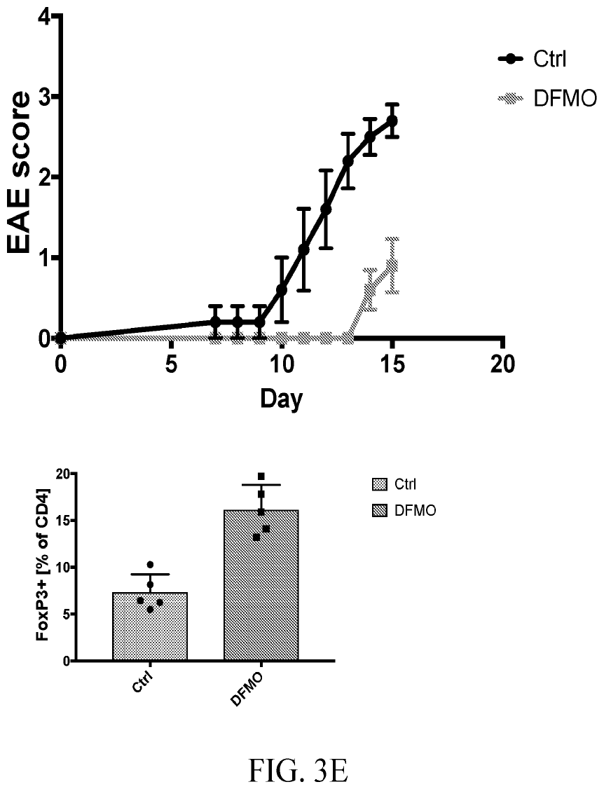

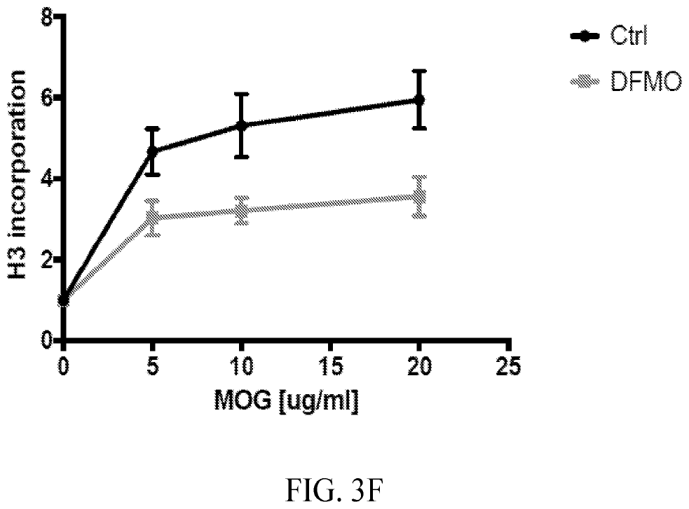

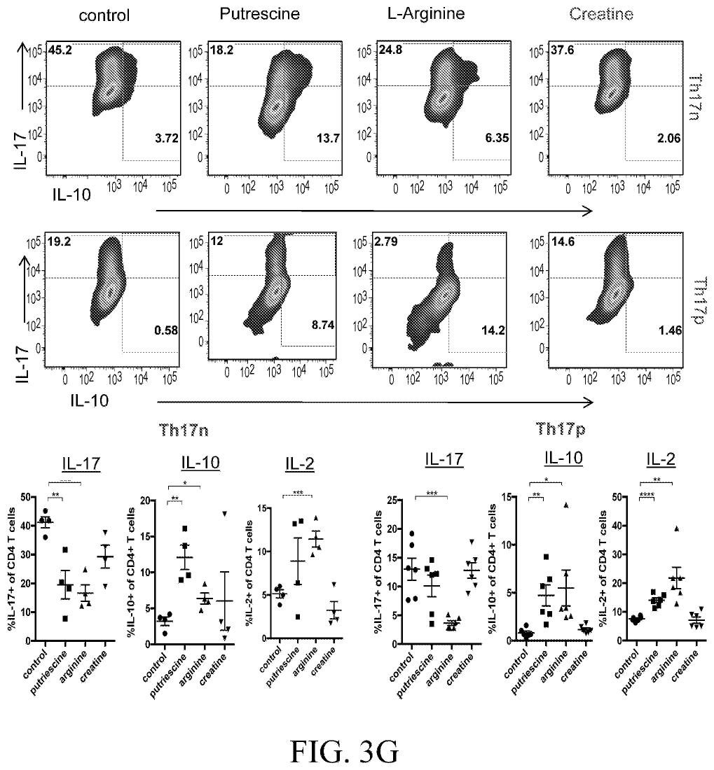

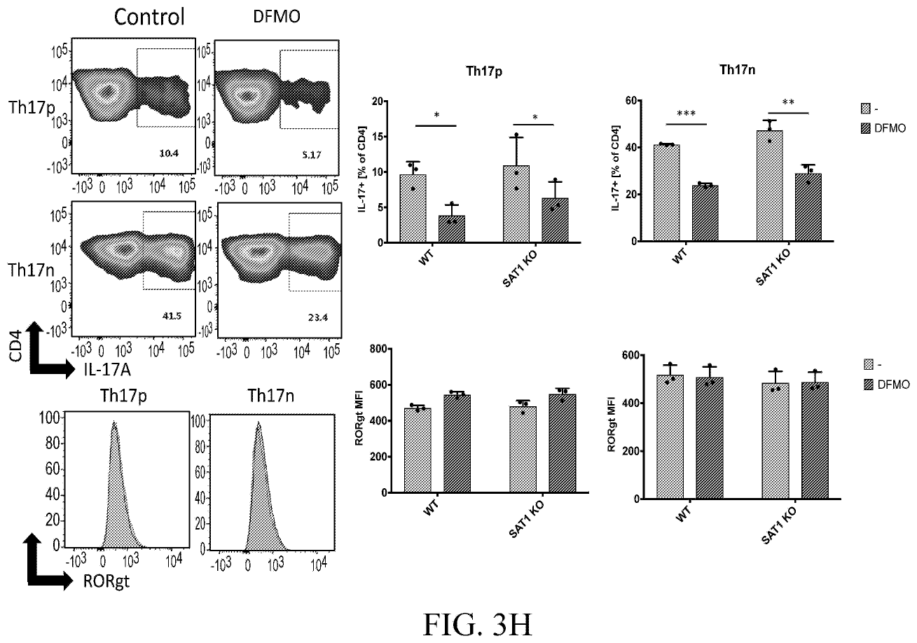

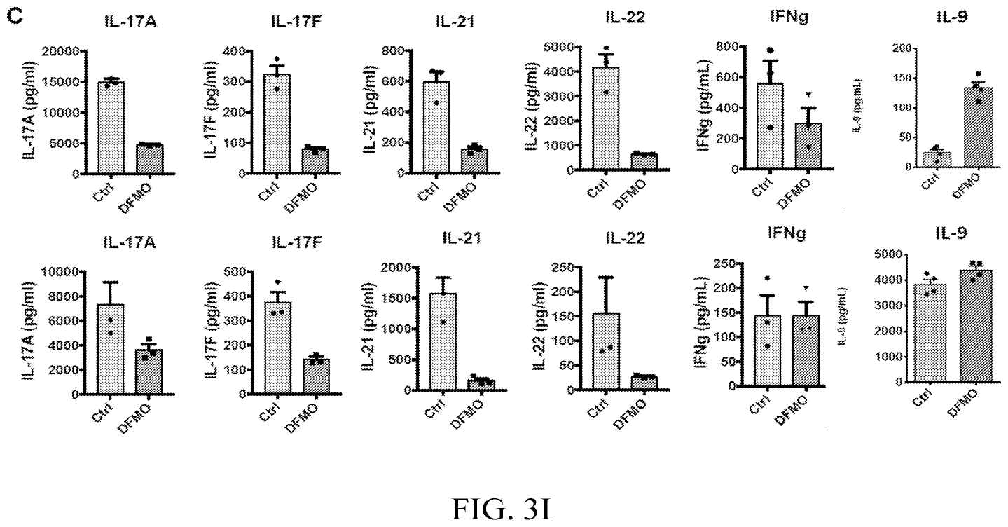

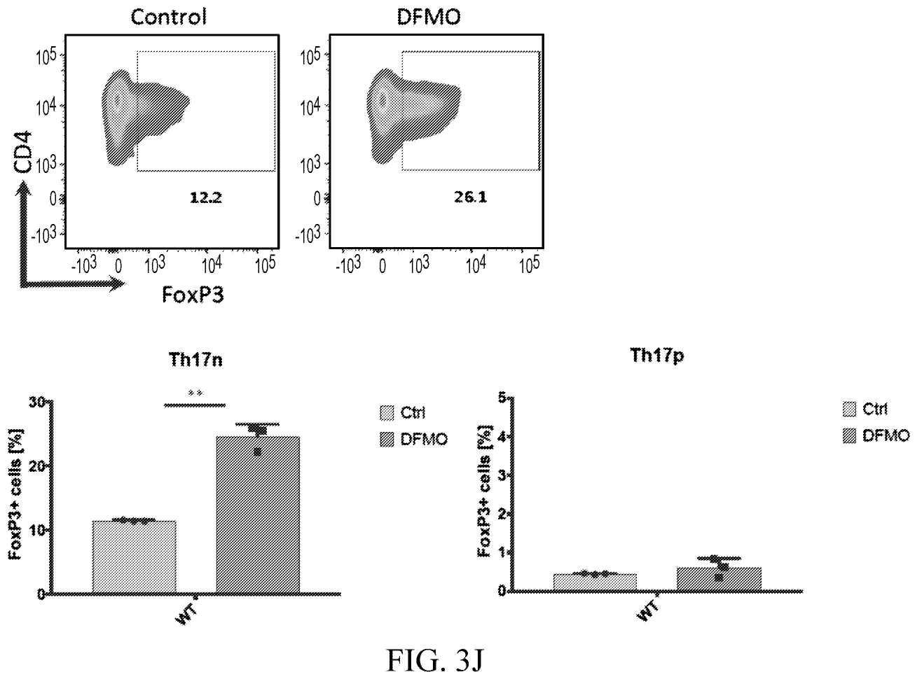

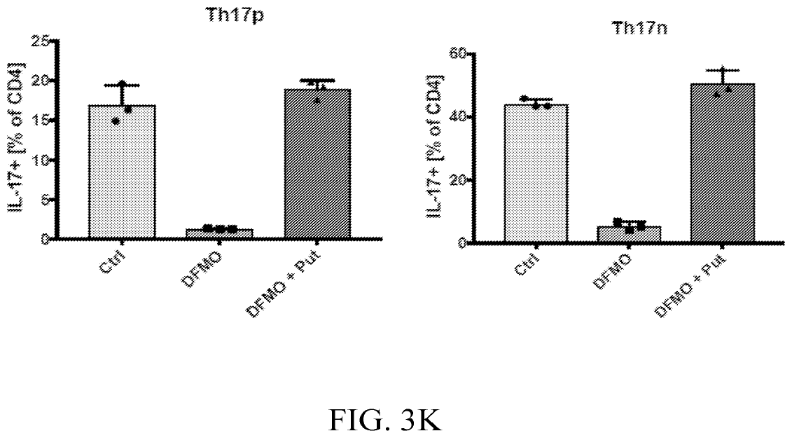

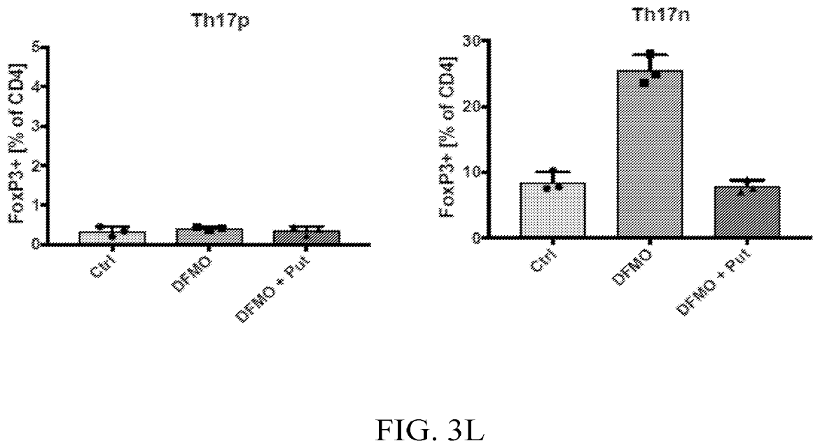

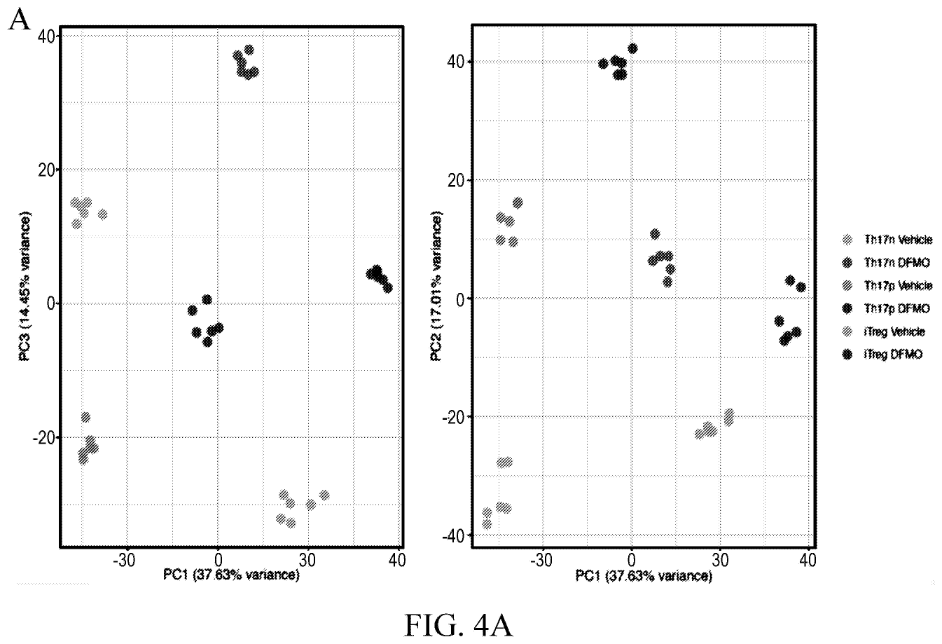

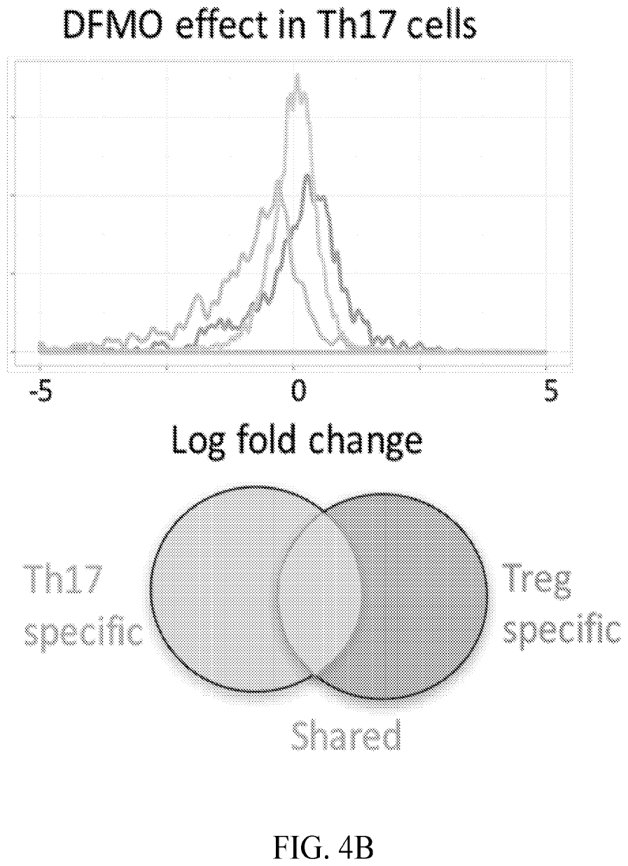

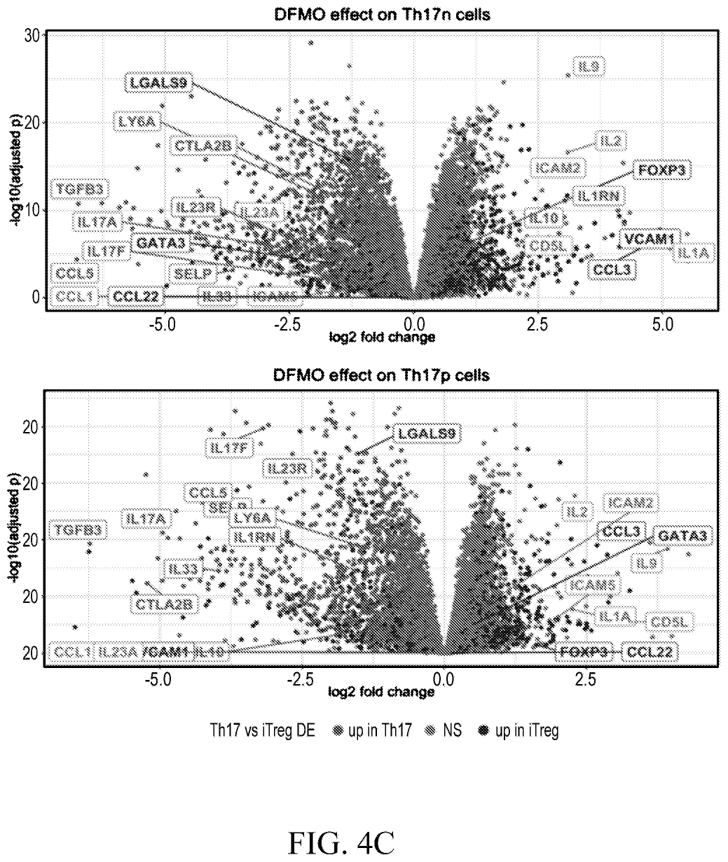

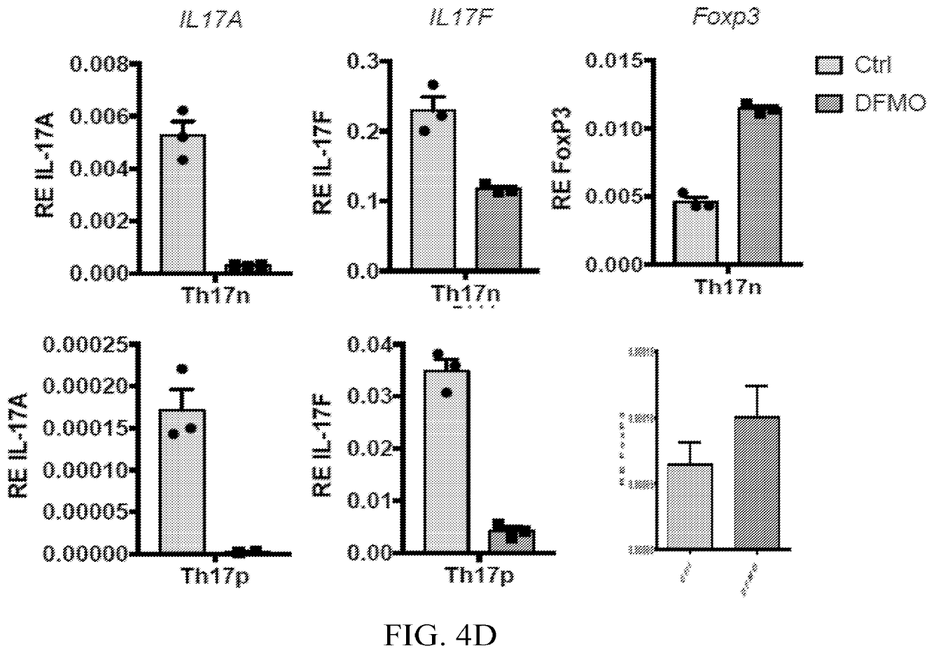

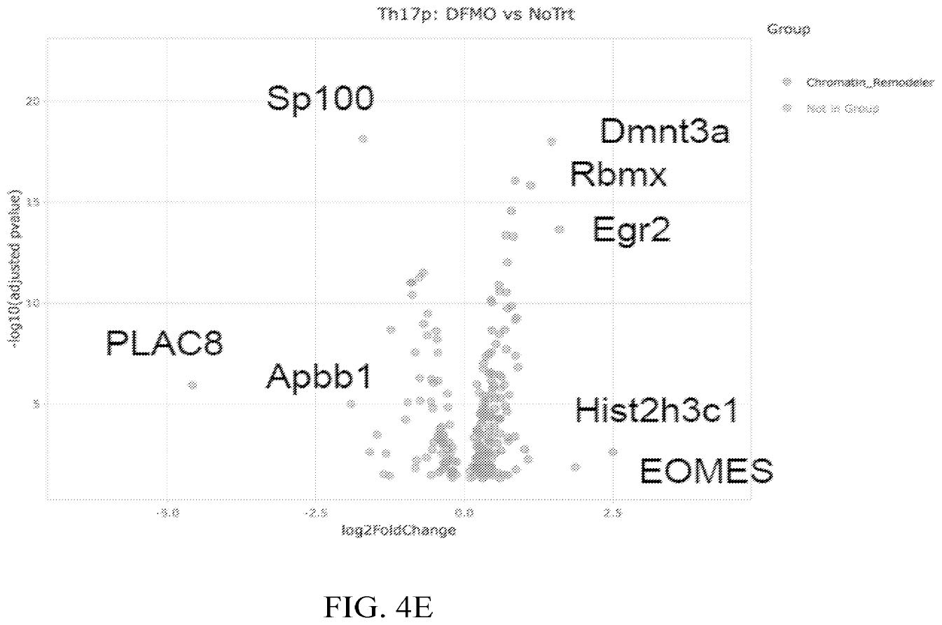

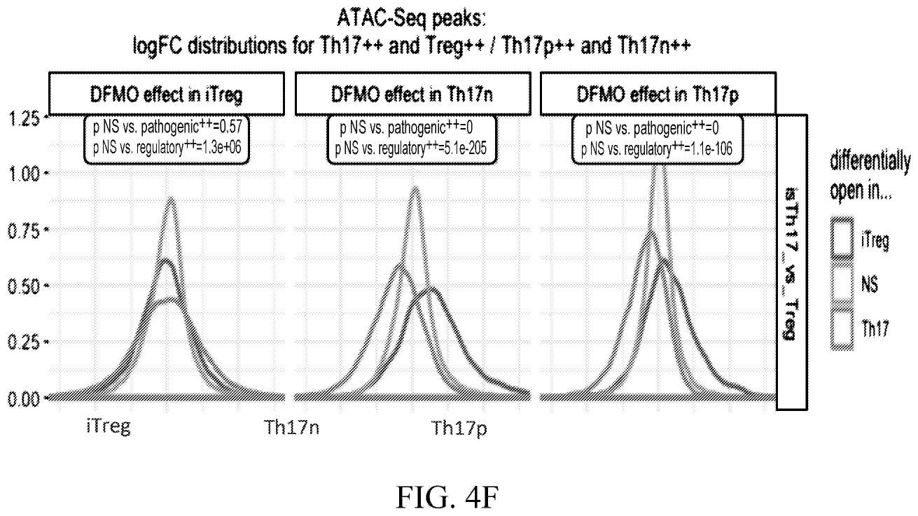

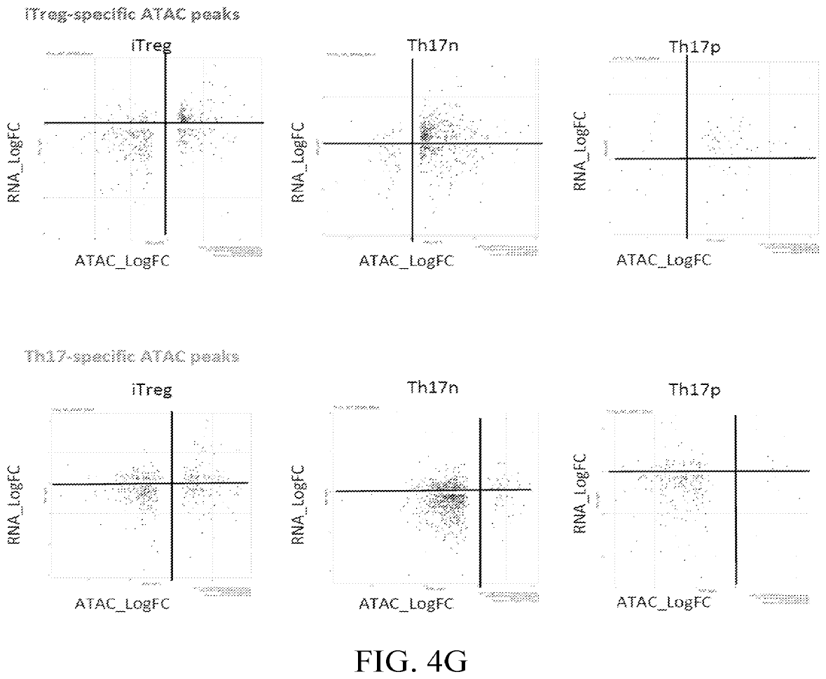

In some embodiments, the prime editing system can be a PE1 system or variant thereof, a PE2 system or variant thereof, or a PE3 (e.g. PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class 1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 3 (from Gleditzsch et al. 2019) below shows several Cas polypeptides and the PAM sequence they recognize.

TABLE 3
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) | TTTV |
| (including LbCpf1 | |
| and A sCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155 (Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016.Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′ end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated herein by reference.

### Sequences Related to Nucleus Targeting and Transportation

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO:1) or PKKKRKVEAS (SEQ ID NO:2); the NLS from nucleoplasmin (e.g., the nucleoplasmin bipartite NLS with the sequence KRPAATKKAGQAKKKK (SEQ ID NO:3)); the c-myc NLS having the amino acid sequence PAAKRVKLD (SEQ ID NO:4) or RQRRNELKRSP (SEQ ID NO:5); the hRNPA1 M9 NLS having the sequence NQSSNFGPMKGGNFGGRSSGPYGGGGQYFAKPRNQGGY (SEQ ID NO:6); the sequence RMRIZFKNKGKDTAELRRRRVEVSVELRKAKKDEQILKRRNV (SEQ ID NO:7) of the IBB domain from importin-alpha; the sequences VSRKRPRP (SEQ ID NO:8) and PPKKARED (SEQ ID NO:9) of the myoma T protein; the sequence PQPKKKPL (SEQ ID NO:10) of human p53; the sequence SALIKKKKKMAP (SEQ ID NO:11) of mouse c-abl IV; the sequences DRLRR (SEQ ID NO:12) and PKQKKRK (SEQ ID NO:13) of the influenza virus NS1; the sequence RKLKKKIKKL (SEQ ID NO:14) of the Hepatitis virus delta antigen; the sequence REKKKFLKRR (SEQ ID NO:15) of the mouse Mx1 protein; the sequence KRKGDEVDGVDEVAKKKSKK (SEQ ID NO:16) of the human poly(ADP-ribose) polymerase; and the sequence RKCLQAGMNLEARKTKK (SEQ ID NO:17) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g. aptamers) for adapter proteins, which may be linked to or fused to a nucleotide deaminase or catalytic domain thereof. When such a guide forms a CRISPR complex (e.g., CRISPR-Cas protein binding to guide and target) the adapter proteins bind and, the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g. due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprise a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include a sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequence (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 by in length.

Suzuki et al. describe in vivo genome editing via CRISPR/Cas9 mediated homology-independent targeted integration (2016, Nature 540:144-149).

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁—(X₁₂X₁₃)—X₁₄₋₃₃ or ₃₄ or ₃₅, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁—(X₁₂X₁₃)—X₁₄₋₃₃ or ₃₄ or ₃₅)z, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 18)
M D P I R S R T P S P A R E L L S G P Q P D G V Q P
T A D R G V S P P A G G P L D G L P A R R T M S R T
R L P S P P A P S P A F S A D S F S D L L R Q F D P
S L F N T S L F D S L P P F G A H H T E A A T G E W
D E V Q S G L R A A D A P P P T M R V A V T A A R P
P R A K P A P R R R A A Q P S D A S P A A Q V D L R
T L G Y S Q Q Q Q E K I K P K V R S T V A Q H H E A
L V G H G F T H A H I V A L S Q H P A A L G T V A V
K Y Q D M I A A L P E A T H E A I V G V G K Q W S G
A R A L E A L L T V A G E L R G P P L Q L D T G Q L
L K I A K R G G V T A V E A V H A W R N A L T G A P
L N

An exemplary amino acid sequence of a C-terminal capping region is:

(SEQ ID NO: 19)
R P A L E S I V A Q L S R P D P A L A A L T N D H L
V A L A C L G G R P A L D A V K K G L P H A P A L I
K R T N R R I P E R T S H R V A D H A Q V V R V L G
F F Q C H S H P A Q A F D D A M T Q F G M S R H G L
L Q L F R R V G V T E L E A R S G T L P P A S Q R W
D R I L Q A S G M K R A K P S P T S T Q T P D Q A S
L H A F A D S L E R D L D A P S P M H E G D Q T R A
S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

### Antibodies

In certain embodiments, the one or more agents is an antibody. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, lgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains. The “variable” domains of an antibody light chain are referred to interchangeably as “light chain variable regions”, “light chain variable domains”, “VL” regions or “VL” domains. The “variable” domains of an antibody heavy chain are referred to interchangeably as “heavy chain constant regions”, “heavy chain constant domains”, “VH” regions or “VH” domains.

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins—harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having V_{H} and CH1 domains and one or more cysteine residues at the C-terminus of the CHI domain; (v) the Fv fragment having the VL and VH domains of a single arm of an antibody; (vi) the dAb fragment (Ward et al., 341 Nature 544 (1989)) which consists of a VH domain or a VL domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}—Cₕ1—V_{H}—-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Aptamers

In certain embodiments, the one or more agents is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-O-methyl (2′-OMe) substituents. Modifications of aptamers may also include, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, 0-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colo.). In certain embodiments, the present invention may utilize any aptamer containing any modification as described herein.

### Diseases and Conditions

### Inflammatory and Autoimmune Diseases

In certain embodiments, modulation of T cell balance may be used to treat inflammatory diseases, disorders or aberrant autoimmune responses. Specific autoimmune responses resulting from an immunotherapy is described further herein. As used throughout the present specification, the terms “autoimmune disease” or “autoimmune disorder” used interchangeably refer to a diseases or disorders caused by an immune response against a self-tissue or tissue component (self-antigen) and include a self-antibody response and/or cell-mediated response. The terms encompass organ-specific autoimmune diseases, in which an autoimmune response is directed against a single tissue, as well as non-organ specific autoimmune diseases, in which an autoimmune response is directed against a component present in two or more, several or many organs throughout the body.

Examples of autoimmune diseases include but are not limited to acute disseminated encephalomyelitis (ADEM); Addison's disease; ankylosing spondylitis; antiphospholipid antibody syndrome (APS); aplastic anemia; autoimmune gastritis; autoimmune hepatitis; autoimmune thrombocytopenia; Behcet's disease; coeliac disease; dermatomyositis; diabetes mellitus type I; Goodpasture's syndrome; Graves' disease; Guillain-Barré syndrome (GBS); Hashimoto's disease; idiopathic thrombocytopenic purpura; inflammatory bowel disease (IBD) including Crohn's disease and ulcerative colitis; mixed connective tissue disease; multiple sclerosis (MS); myasthenia gravis; opsoclonus myoclonus syndrome (OMS); optic neuritis; Ord's thyroiditis; pemphigus; pernicious anaemia; polyarteritis nodosa; polymyositis; primary biliary cirrhosis; primary myoxedema; psoriasis; rheumatic fever; rheumatoid arthritis; Reiter's syndrome; scleroderma; Sjögren's syndrome; systemic lupus erythematosus; Takayasu's arteritis; temporal arteritis; vitiligo; warm autoimmune hemolytic anemia; or Wegener's granulomatosis.

Examples of inflammatory diseases or disorders include, but are not limited to, asthma, allergy, allergic rhinitis, allergic airway inflammation, atopic dermatitis (AD), chronic obstructive pulmonary disease (COPD), inflammatory bowel disease (IBD), Irritable bowel syndrome (IBS), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome).

The asthma may be allergic asthma, non-allergic asthma, severe refractory asthma, asthma exacerbations, viral-induced asthma or viral-induced asthma exacerbations, steroid resistant asthma, steroid sensitive asthma, eosinophilic asthma or non-eosinophilic asthma and other related disorders characterized by airway inflammation or airway hyperresponsiveness (AHR).

The COPD may be a disease or disorder associated in part with, or caused by, cigarette smoke, air pollution, occupational chemicals, allergy or airway hyperresponsiveness.

The allergy may be associated with foods, pollen, mold, dust mites, animals, or animal dander.

The IBD may be ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon.

The arthritis may be selected from the group consisting of osteoarthritis, rheumatoid arthritis and psoriatic arthritis.

### Checkpoint Blockade Therapy

Immunotherapy can include checkpoint blockers (CBP), chimeric antigen receptors (CARs), and adoptive T-cell therapy. Antibodies that block the activity of checkpoint receptors, including CTLA-4, PD-1, Tim-3, Lag-3, and TIGIT, either alone or in combination, have been associated with improved effector CD8⁺ T cell responses in multiple pre-clinical cancer models (Johnston et al., 2014. The immunoreceptor TIGIT regulates antitumor and antiviral CD8(+) T cell effector function. Cancer cell 26, 923-937; Ngiow et al., 2011. Anti-TIM3 antibody promotes T cell IFN-gamma-mediated antitumor immunity and suppresses established tumors. Cancer research 71, 3540-3551; Sakuishi et al., 2010. Targeting Tim-3 and PD-1 pathways to reverse T cell exhaustion and restore anti-tumor immunity. The Journal of experimental medicine 207, 2187-2194; and Woo et al., 2012. Immune inhibitory molecules LAG-3 and PD-1 synergistically regulate T-cell function to promote tumoral immune escape. Cancer research 72, 917-927). Similarly, blockade of CTLA-4 and PD-1 in patients (Brahmer et al., 2012. Safety and activity of anti-PD-L1 antibody in patients with advanced cancer. The New England journal of medicine 366, 2455-2465; Hodi et al., 2010. Improved survival with ipilimumab in patients with metastatic melanoma. The New England journal of medicine 363, 711-723; Schadendorf et al., 2015. Pooled Analysis of Long-Term Survival Data From Phase II and Phase III Trials of Ipilimumab in Unresectable or Metastatic Melanoma. Journal of clinical oncology: official journal of the American Society of Clinical Oncology 33, 1889-1894; Topalian et al., 2012. Safety, activity, and immune correlates of anti-PD-1 antibody in cancer. The New England journal of medicine 366, 2443-2454; and Wolchok et al., 2017. Overall Survival with Combined Nivolumab and Ipilimumab in Advanced Melanoma. The New England journal of medicine 377, 1345-1356) has shown increased frequencies of proliferating T cells, often with specificity for tumor antigens, as well as increased CD8⁺ T cell effector function (Ayers et al., 2017. IFN-gamma-related mRNA profile predicts clinical response to PD-1 blockade. The Journal of clinical investigation 127, 2930-2940; Das et al., 2015. Combination therapy with anti-CTLA-4 and anti-PD-1 leads to distinct immunologic changes in vivo. Journal of immunology 194, 950-959; Gubin et al., 2014. Checkpoint blockade cancer immunotherapy targets tumour-specific mutant antigens. Nature 515, 577-581; Huang et al., 2017. T-cell invigoration to tumour burden ratio associated with anti-PD-1 response. Nature 545, 60-65; Kamphorst et al., 2017. Proliferation of PD-1+CD8 T cells in peripheral blood after PD-1-targeted therapy in lung cancer patients. Proceedings of the National Academy of Sciences of the United States of America 114, 4993-4998; Kvistborg et al., 2014. Anti-CTLA-4 therapy broadens the melanoma-reactive CD8+ T cell response. Science translational medicine 6, 254ra128; van Rooij et al., 2013. Tumor exome analysis reveals neoantigen-specific T-cell reactivity in an ipilimumab-responsive melanoma. Journal of clinical oncology: official journal of the American Society of Clinical Oncology 31, e439-442; and Yuan et al., 2008. CTLA-4 blockade enhances polyfunctional NY-ESO-1 specific T cell responses in metastatic melanoma patients with clinical benefit. Proceedings of the National Academy of Sciences of the United States of America 105, 20410-20415). Accordingly, the success of checkpoint receptor blockade has been attributed to the binding of blocking antibodies to checkpoint receptors expressed on dysfunctional CD8⁺ T cells and restoring effector function in these cells. The check point blockade therapy may be an inhibitor of any check point protein described herein. The checkpoint blockade therapy may comprise anti-TIM3, anti-CTLA4, anti-PD-L1, anti-PD1, anti-TIGIT, anti-LAG3, or combinations thereof. Anti-PD1 antibodies are disclosed in U.S. Pat. No. 8,735,553. Antibodies to LAG-3 are disclosed in U.S. Pat. No. 9,132,281. Anti-CTLA4 antibodies are disclosed in U.S. Pat. Nos. 9,327,014, 9,320,811, and 9,062,111. Specific check point inhibitors include, but are not limited to anti-CTLA4 antibodies (e.g., Ipilimumab and tremelimumab), anti-PD-1 antibodies (e.g., Nivolumab, Pembrolizumab), and anti-PD-L1 antibodies (e.g., Atezolizumab).

In certain embodiments, immunotherapy leads to immune-related adverse events (irAEs) (see, e.g., Byun et al., (2017) Cancer immunotherapy—immune checkpoint blockade and associated endocrinopathies. Nat Rev Endocrinol. 2017 April; 13(4): 195-207; Abdel-Wahab et al., (2016) Adverse Events Associated with Immune Checkpoint Blockade in Patients with Cancer: A Systematic Review of Case Reports. PLoS ONE 11 (7): e0160221. doi:10.1371/journal.pone.0160221; and Gelao et al., Immune Checkpoint Blockade in Cancer Treatment: A Double-Edged Sword Cross-Targeting the Host as an “Innocent Bystander”, Toxins 2014, 6, 914-933; doi:10.3390/toxins6030914). Thus, patients receiving immunotherapy are at risk for adverse autoimmune responses.

In certain embodiments, irAEs are related to Th17 pathogenicity. In one study, patients treated with ipilimumab had fluctuations in serum IL-17 levels, such that serum IL-17 levels in patients with colitis versus no irAEs demonstrated significantly higher serum IL-17 levels in the patients with colitis (Callahan et al., (2011) Evaluation of serum IL-17 levels during ipilimumab therapy: Correlation with colitis. Journal of Clinical Oncology 29, no. 15 suppl 2505-2505).

In certain embodiments, the modulating agents described herein can be used to shift T cell balance away from Th17 autoimmune responses in patients treated with checkpoint blockade therapy. In certain embodiments, agents modulating the polyamine pathway or glycolysis pathway are used as part of a cancer therapy regimen.

### Adoptive Cell Transfer

In certain embodiments, T cells differentiated according to the present invention (e.g., treated with DFMO, a polyamine, or other polyamine analogue, polyamine pathway targeting drug, glycolysis targeting drug, or genetically modified) are used in adoptive cell transfer to treat an aberrant inflammatory response (e.g., autoimmune response). In certain embodiments, a modulating agent according to the present invention is used in combination with ACT to prevent an aberrant immune response.

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostate; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDG1cp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAXS); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (Dl), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CIVIL), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR a and chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728, 5,851,828, 5,912,170, 6,004,811, 6,284,240, 6,392,013, 6,410,014, 6,753,162, 8,211,422, and International Patent Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3 or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/0X_{40/4}-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI la-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682, 8,399,645, 5,686,281, and International Patent Publication Nos. WO2014134165 and WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIM, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3 chain (such as amino acid residues 52-163 of the human CD3 zeta chain, as shown in SEQ ID NO:14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO:6 of U.S. Pat. No. 7,446,190; these can include the following portion of CD28 as set forth in Genbank identifier NM_006139 (sequence version 1, 2 or 3):

(SEQ ID NO: 20)
IEVMYPPPYLDNEKSNGTIIHVKGKHLCPSPLFPGPSKPFWVLVVVGGVLA
CYSLLVTVAFIIFWVRSKRSRLLHSDYMNMTPRRPGPTRKHYQPYAPPRDF
AAYRS)).

Alternatively, when the zeta sequence lies between the CD28 sequence and the antigen-binding element, intracellular domain of CD28 can be used alone (such as amino sequence set forth in SEQ ID NO:9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO:6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-molecule. The exact sequence of the CD28 molecule included in the FMC63-28Z CAR corresponded to Genbank identifier NM_006139; the sequence included all amino acids starting with the amino acid sequence IEVMYPPPY (SEQ ID NO:21) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO:21) and continuing all the way to the carboxy-terminus of the protein. The sequence is reproduced herein:

(SEQ ID NO: 22)
IEVMYPPPYLDNEKSNGTIIHVKGKHLCPSPLFPGPSKPFWVLVVVGGVLA
FCYSLLVTVAIIFWVRSKRSRLLHSDYMNMTPRRPGPTRKHYQPYAPPRDF
AAYRS.

Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in International Patent Publication WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD3; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRT gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signaling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J.Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US Patent Publication Nos. US 20160046724A1, US 20180085444 A1, and US 20170283504 A1, and International Patent Publications No. WO2016014789A2, WO2017211900A1, WO2015158671A1, WO2018028647A1, and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may be eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., International Patent Publication Nos. WO 2013/044225, WO 2016/000304, WO 2015/057834, WO 2015/057852, and WO 2016/070061, U.S. Pat. No. 9,233,125, and US Patent Publication No. US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US Patent Publication Nos. US 2015/0368342, US 2016/0175359, and US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-y). CART cells of this kind may for example be used in animal models, for example to treat tumor xenografts.

In certain embodiments, ACT includes co-transferring CD4+Th1 cells and CD8+ CTLs to induce a synergistic antitumor response (see, e.g., Li et al., Adoptive cell therapy with CD4+T helper 1 cells and CD8+ cytotoxic T cells enhances complete rejection of an established tumor, leading to generation of endogenous memory responses to non-targeted tumor epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4⁺ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective number of cells are administrated as a single dose. In another embodiment, the effective number of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective number of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CART cells, Sci Transl Med. 2017 Jan. 25; 9 (374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more WIC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see International Patent Publications WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

International Patent Publication No. WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, International Patent Publication No. WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. International Patent Publication No. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694, 6,534,055, 6,905,680, 5,858,358, 6,887,466, 6,905,681, 7,144,575, 7,232,566, 7,175,843, 5,883,223, 6,905,874, 6,797,514, 6,867,041, and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g., via magnetic selection) the antigen specific cells prior to or following one or two rounds of expansion. Sorting or positively selecting antigen-specific cells can be carried out using peptide-MEW tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MEW molecule of interest and any antigen of interest as described herein. Specific epitopes to be used in this context can be identified using numerous assays known in the art. For example, the ability of a polypeptide to bind to MEW class I may be evaluated indirectly by monitoring the ability to promote incorporation of ¹²⁵I labeled β2-microglobulin (β2m) into MHC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in International Patent Publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in International Patent Publication No. WO 2003057171, U.S. Pat. No. 8,034,334, and U.S. Patent Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m²/day.

### Diagnostic Methods

In certain embodiments, polyamines or enzymes of the polyamine pathway are used as biomarkers to detect an immune response (e.g., any disease or condition described herein). In certain embodiments, increased polyamines or specific enzymes (e.g., SAT1) indicate an inflammatory response, such as an autoimmune response. Detection of polyamines or enzymes of the polyamine pathway may be used in diagnosing, prognosing or monitoring a disease an immune response.

The terms “diagnosis” and “monitoring” are commonplace and well-understood in medical practice. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The term “monitoring” generally refers to the follow-up of a disease or a condition in a subject for any changes which may occur over time.

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

### Biomarkers

The term “biomarker” is widespread in the art and commonly broadly denotes a biological molecule, more particularly an endogenous biological molecule, and/or a detectable portion thereof, whose qualitative and/or quantitative evaluation in a tested object (e.g., in or on a cell, cell population, tissue, organ, or organism, e.g., in a biological sample of a subject) is predictive or informative with respect to one or more aspects of the tested object's phenotype and/or genotype (e.g., detecting polyamines). The terms “marker” and “biomarker” may be used interchangeably throughout this specification. Biomarkers as intended herein may be metabolites (e.g., polyamines), nucleic acid-based or peptide-, polypeptide- and/or protein-based. For example, a marker may be comprised of peptide(s), polypeptide(s) and/or protein(s) encoded by a given gene, or of detectable portions thereof. Further, whereas the term “nucleic acid” generally encompasses DNA, RNA and DNA/RNA hybrid molecules, in the context of markers the term may typically refer to heterogeneous nuclear RNA (hnRNA), pre-mRNA, messenger RNA (mRNA), or complementary DNA (cDNA), or detectable portions thereof. Such nucleic acid species are particularly useful as markers, since they contain qualitative and/or quantitative information about the expression of the gene. Particularly preferably, a nucleic acid-based marker may encompass mRNA of a given gene, or cDNA made of the mRNA, or detectable portions thereof. Any such nucleic acid(s), peptide(s), polypeptide(s) and/or protein(s) encoded by or produced from a given gene are encompassed by the term “gene product(s)”.

Preferably, markers as intended herein may be extracellular or cell surface markers (e.g., metabolites), as methods to measure extracellular or cell surface marker(s) need not disturb the integrity of the cell membrane and may not require fixation/permeabilization of the cells.

Unless otherwise apparent from the context, reference herein to any marker, such as a metabolite, peptide, polypeptide, protein, or nucleic acid, may generally also encompass modified forms of said marker, such as bearing post-expression modifications including, for example, phosphorylation, glycosylation, lipidation, methylation, cysteinylation, sulphonation, glutathionylation, acetylation, oxidation of methionine to methionine sulphoxide or methionine sulphone, and the like.

The term “peptide” as used throughout this specification preferably refers to a polypeptide as used herein consisting essentially of 50 amino acids or less, e.g., 45 amino acids or less, preferably 40 amino acids or less, e.g., 35 amino acids or less, more preferably 30 amino acids or less, e.g., 25 or less, 20 or less, 15 or less, 10 or less or 5 or less amino acids.

The term “polypeptide” as used throughout this specification generally encompasses polymeric chains of amino acid residues linked by peptide bonds. Hence, insofar a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally-occurring polypeptide parts that ensue from processing of such full-length polypeptides.

The term “protein” as used throughout this specification generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native protein, such as, e.g., amino acid deletions, additions and/or substitutions. The term contemplates both full-length proteins and protein parts or fragments, e.g., naturally-occurring protein parts that ensue from processing of such full-length proteins.

The reference to any marker, including any metabolite, peptide, polypeptide, protein, or nucleic acid, corresponds to the marker commonly known under the respective designations in the art. The terms encompass such markers of any organism where found, and particularly of animals, preferably warm-blooded animals, more preferably vertebrates, yet more preferably mammals, including humans and non-human mammals, still more preferably of humans.

The terms particularly encompass such markers, including any metabolites, peptides, polypeptides, proteins, or nucleic acids, with a native sequence, i.e., ones of which the primary sequence is the same as that of the markers found in or derived from nature. A skilled person understands that native sequences may differ between different species due to genetic divergence between such species. Moreover, native sequences may differ between or within different individuals of the same species due to normal genetic diversity (variation) within a given species. Also, native sequences may differ between or even within different individuals of the same species due to somatic mutations, or post-transcriptional or post-translational modifications. Any such variants or isoforms of markers are intended herein. Accordingly, all sequences of markers found in or derived from nature are considered “native”. The terms encompass the markers when forming a part of a living organism, organ, tissue or cell, when forming a part of a biological sample, as well as when at least partly isolated from such sources. The terms also encompass markers when produced by recombinant or synthetic means.

In certain embodiments, markers, including any metabolites, peptides, polypeptides, proteins, or nucleic acids, may be human, i.e., their primary sequence may be the same as a corresponding primary sequence of or present in a naturally occurring human markers. Hence, the qualifier “human” in this connection relates to the primary sequence of the respective markers, rather than to their origin or source. For example, such markers may be present in or isolated from samples of human subjects or may be obtained by other means (e.g., by recombinant expression, cell-free transcription or translation, or non-biological nucleic acid or peptide synthesis).

The reference herein to any marker, including any metabolite, peptide, polypeptide, protein, or nucleic acid, also encompasses fragments thereof. Hence, the reference herein to measuring (or measuring the quantity of) any one marker may encompass measuring the marker and/or measuring one or more fragments thereof.

For example, any marker and/or one or more fragments thereof may be measured collectively, such that the measured quantity corresponds to the sum amounts of the collectively measured species. In another example, any marker and/or one or more fragments thereof may be measured each individually. The terms encompass fragments arising by any mechanism, in vivo and/or in vitro, such as, without limitation, by alternative transcription or translation, exo- and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

The term “fragment” as used throughout this specification with reference to a peptide, polypeptide, or protein generally denotes a portion of the peptide, polypeptide, or protein, such as typically an N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of 5 consecutive amino acids, or 10 consecutive amino acids, or 20 consecutive amino acids, or 30 consecutive amino acids, e.g., ≥10 consecutive amino acids, such as for example 50 consecutive amino acids, e.g., 60, 70, 80, 90, 100, 200, 300, 400, 500 or 600 consecutive amino acids of the corresponding full-length peptide, polypeptide, or protein.

The term “fragment” as used throughout this specification with reference to a nucleic acid (polynucleotide) generally denotes a 5′- and/or 3′-truncated form of a nucleic acid. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the nucleic acid sequence length of said nucleic acid. For example, insofar not exceeding the length of the full-length nucleic acid, a fragment may include a sequence of ≥5 consecutive nucleotides, or ≥10 consecutive nucleotides, or ≥20 consecutive nucleotides, or ≥30 consecutive nucleotides, e.g., ≥40 consecutive nucleotides, such as for example ≥50 consecutive nucleotides, e.g., ≥60, ≥70, ≥80, ≥90, ≥100, ≥200, ≥300, ≥400, ≥500 or ≥600 consecutive nucleotides of the corresponding full-length nucleic acid.

Cells such as immune cells as disclosed herein may in the context of the present specification be said to “comprise the expression” or conversely to “not express” one or more markers, such as one or more genes or gene products; or be described as “positive” or conversely as “negative” for one or more markers, such as one or more genes or gene products; or be said to “comprise” a defined “gene or gene product signature”.

Such terms are commonplace and well-understood by the skilled person when characterizing cell phenotypes. By means of additional guidance, when a cell is said to be positive for or to express or comprise expression of a given marker, such as a given gene or gene product, a skilled person would conclude the presence or evidence of a distinct signal for the marker when carrying out a measurement capable of detecting or quantifying the marker in or on the cell. Suitably, the presence or evidence of the distinct signal for the marker would be concluded based on a comparison of the measurement result obtained for the cell to a result of the same measurement carried out for a negative control (for example, a cell known to not express the marker) and/or a positive control (for example, a cell known to express the marker). Where the measurement method allows for a quantitative assessment of the marker, a positive cell may generate a signal for the marker that is at least 1.5-fold higher than a signal generated for the marker by a negative control cell or than an average signal generated for the marker by a population of negative control cells, e.g., at least 2-fold, at least 4-fold, at least 10-fold, at least 20-fold, at least 30-fold, at least 40-fold, at least 50-fold higher or even higher. Further, a positive cell may generate a signal for the marker that is 3.0 or more standard deviations, e.g., 3.5 or more, 4.0 or more, 4.5 or more, or 5.0 or more standard deviations, higher than an average signal generated for the marker by a population of negative control cells.

A marker, for example a gene or gene product, for example a peptide, polypeptide, protein, or nucleic acid, or a group of two or more markers, is “detected” or “measured” in a tested object (e.g., in or on a cell, cell population, tissue, organ, or organism, e.g., in a biological sample of a subject) when the presence or absence and/or quantity of said marker or said group of markers is detected or determined in the tested object, preferably substantially to the exclusion of other molecules and analytes, e.g., other genes or gene products.

The terms “increased” or “increase” or “upregulated” or “upregulate” as used herein generally mean an increase by a statically significant amount. For avoidance of doubt, “increased” means a statistically significant increase of at least 10% as compared to a reference level, including an increase of at least 20%, at least 30%, at least 40%, at least 50%, at least 60%, at least 70%, at least 80%, at least 90%, at least 100% or more, including, for example at least 2-fold, at least 3-fold, at least 4-fold, at least 5-fold, at least 10-fold increase or greater as compared to a reference level, as that term is defined herein.

The term “reduced” or “reduce” or “decrease” or “decreased” or “downregulate” or “downregulated” as used herein generally means a decrease by a statistically significant amount relative to a reference. For avoidance of doubt, “reduced” means statistically significant decrease of at least 10% as compared to a reference level, for example a decrease by at least 20%, at least 30%, at least 40%, at least 50%, or at least 60%, or at least 70%, or at least 80%, at least 90% or more, up to and including a 100% decrease (i.e., absent level as compared to a reference sample), or any decrease between 10-100% as compared to a reference level, as that.

The terms “quantity”, “amount” and “level” are synonymous and generally well-understood in the art. The terms as used throughout this specification may particularly refer to an absolute quantification of a marker in a tested object (e.g., in or on a cell, cell population, tissue, organ, or organism, e.g., in a biological sample of a subject), or to a relative quantification of a marker in a tested object, i.e., relative to another value such as relative to a reference value, or to a range of values indicating a base-line of the marker. Such values or ranges may be obtained as conventionally known.

An absolute quantity of a marker may be advantageously expressed as weight or as molar amount, or more commonly as a concentration, e.g., weight per volume or mol per volume. A relative quantity of a marker may be advantageously expressed as an increase or decrease or as a fold-increase or fold-decrease relative to said another value, such as relative to a reference value. Performing a relative comparison between first and second variables (e.g., first and second quantities) may but need not require determining first the absolute values of said first and second variables. For example, a measurement method may produce quantifiable readouts (such as, e.g., signal intensities) for said first and second variables, wherein said readouts are a function of the value of said variables, and wherein said readouts may be directly compared to produce a relative value for the first variable vs. the second variable, without the actual need to first convert the readouts to absolute values of the respective variables.

Reference values may be established according to known procedures previously employed for other cell populations, biomarkers and gene or gene product signatures. For example, a reference value may be established in an individual or a population of individuals characterized by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥0% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

Detection of a biomarker may be by any means known in the art. Methods of detection include, but are not limited to enzymatic assays, flow cytometry, mass cytometry, fluorescence activated cell sorting (FACS), fluorescence microscopy, affinity separation, magnetic cell separation, microfluidic separation, RNA-seq (e.g., bulk or single cell), quantitative PCR, MERFISH (multiplex (in situ) RNA FISH), immunological assay methods by specific binding between a separable, detectable and/or quantifiable immunological binding agent (antibody) and the marker, mass spectrometry analysis methods, chromatography methods and combinations thereof. Immunological assay methods include without limitation immunohistochemistry, immunocytochemistry, flow cytometry, mass cytometry, fluorescence activated cell sorting (FACS), fluorescence microscopy, fluorescence based cell sorting using microfluidic systems, immunoaffinity adsorption based techniques such as affinity chromatography, magnetic particle separation, magnetic activated cell sorting or bead based cell sorting using microfluidic systems, enzyme-linked immunosorbent assay (ELISA) and ELISPOT based techniques, radioimmunoassay (MA), Western blot, etc. While particulars of chromatography are well known in the art, for further guidance see, e.g., Meyer M., 1998, ISBN: 047198373X, and “Practical HPLC Methodology and Applications”, Bidlingmeyer, B. A., John Wiley & Sons Inc., 1993. Exemplary types of chromatography include, without limitation, high-performance liquid chromatography (HPLC), normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immunoaffinity, immobilized metal affinity chromatography, and the like.

### Mass Spectrometry Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)2 fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854, 5,288,644, 5,324,633, 5,432,049, 5,470,710, 5,492,806, 5,503,980, 5,510,270, 5,525,464, 5,547,839, 5,580,732, 5,661,028, and 5,800,992,the disclosures of which are herein incorporated by reference, as well as International Patent Publication Nos. WO 95/21265, WO 96/31622, WO 97/10365, WO 97/27317, and EP 373203; and EP 785280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

### Sequencing and Single Cell Sequencing

In certain embodiments, the invention involves targeted nucleic acid profiling (e.g., sequencing, quantitative reverse transcription polymerase chain reaction, and the like) (see e.g., Geiss G K, et al., Direct multiplexed measurement of gene expression with color-coded probe pairs. Nat Biotechnol. 2008 March; 26(3):317-25). In certain embodiments, a target nucleic acid molecule (e.g., RNA molecule) may be sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-6′73, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. Jan; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US Patent Publication Nos. US20160208323A1 and US20160060691A1; and International Patent Publication No. WO2017156336A1).

### Administration of Pharmaceutical Compositions

A “pharmaceutical composition” refers to a composition that usually contains an excipient, such as a pharmaceutically acceptable carrier that is conventional in the art and that is suitable for administration to cells or to a subject.

The pharmaceutical composition according to the present invention can, in one alternative, include a prodrug. When a pharmaceutical composition according to the present invention includes a prodrug, prodrugs and active metabolites of a compound may be identified using routine techniques known in the art. (See, e.g., Bertolini et al., J. Med. Chem., 40, 2011-2016 (1997); Shan et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992)).

The term “pharmaceutically acceptable” as used throughout this specification is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilizers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavorings, aromatizers, thickeners, agents for achieving a depot effect, coatings, antifungal agents, preservatives, stabilizers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

The pharmaceutical composition can be applied parenterally, rectally, orally or topically. Preferably, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infusion. The skilled person will understand that compositions which are to be administered orally or topically will usually not comprise cells, although it may be envisioned for oral compositions to also comprise cells, for example when gastro-intestinal tract indications are treated. Each of the cells or active components (e.g., immunomodulants) as discussed herein may be administered by the same route or may be administered by a different route. By means of example, and without limitation, cells may be administered parenterally and other active components may be administered orally.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of their environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

In certain embodiments, a pharmaceutical cell preparation as taught herein may be administered in a form of liquid composition. In embodiments, the cells or pharmaceutical composition comprising such can be administered systemically, topically, within an organ or at a site of organ dysfunction or lesion.

Preferably, the pharmaceutical compositions may comprise a therapeutically effective amount of the specified immune cells and/or other active components (e.g., immunomodulants). The term “therapeutically effective amount” refers to an amount which can elicit a biological or medicinal response in a tissue, system, animal or human that is being sought by a researcher, veterinarian, medical doctor or other clinician, and in particular can prevent or alleviate one or more of the local or systemic symptoms or features of a disease or condition being treated.

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

The amount of the agents which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. Effective doses may be extrapolated from dose-response curves derived from in vitro or animal model test systems. Ultimately the attending physician will decide on the appropriate duration of therapy using compositions of the present invention. Dosage will also vary according to the age, weight and response of the individual patient.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

The invention is further described in the following examples, which do not limit the scope of the invention described in the claims.

### EXAMPLES

### Example 1—Identification of Metabolic Pathways Associated with Th17 Pathogenicity

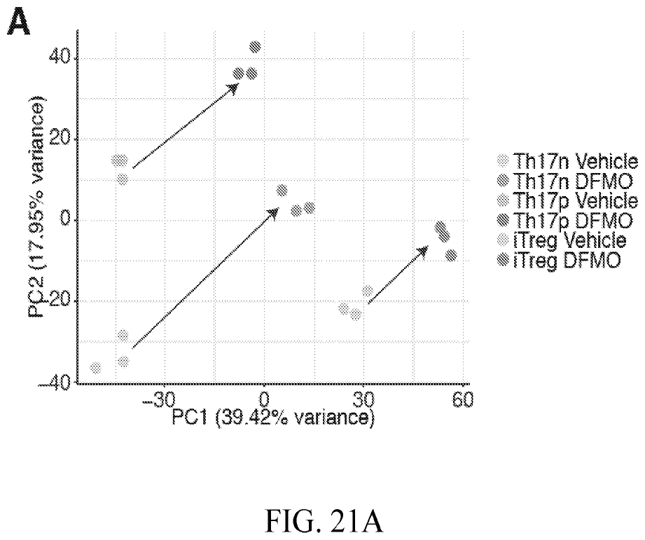

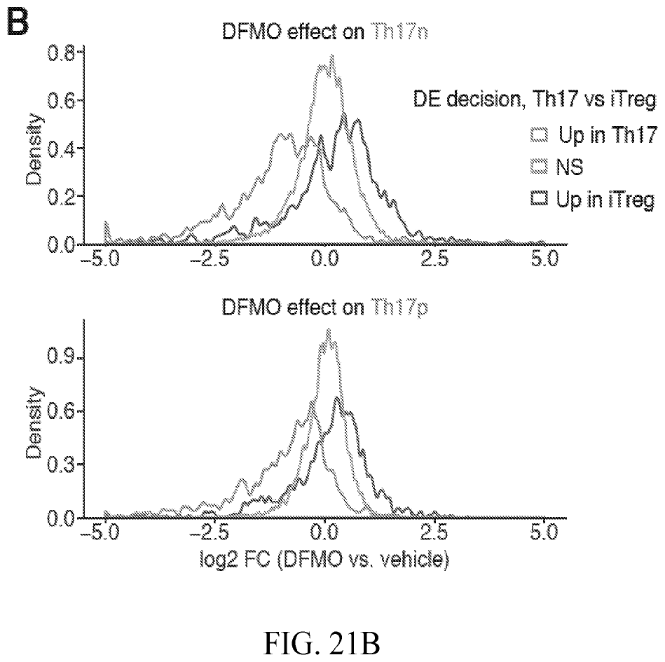

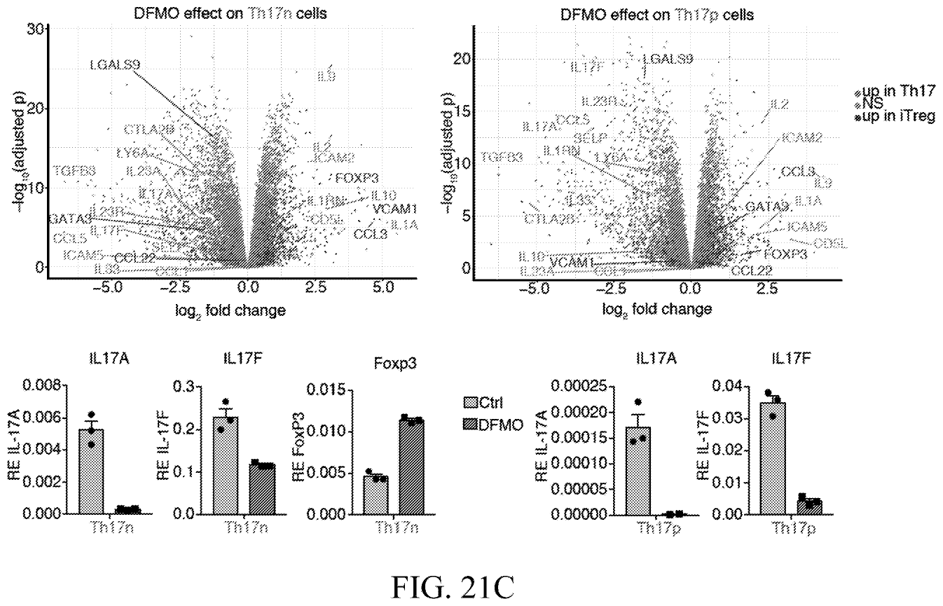

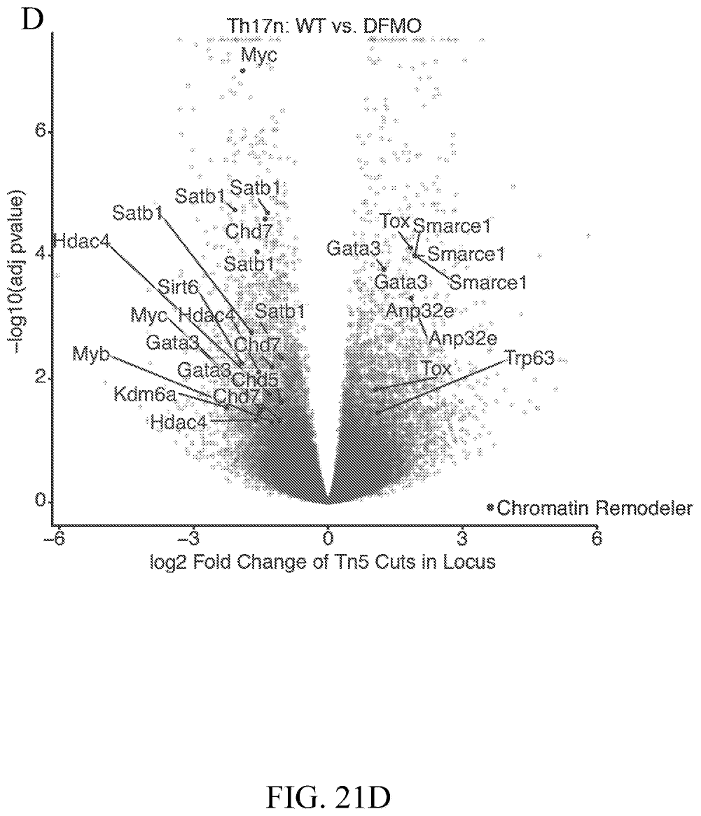

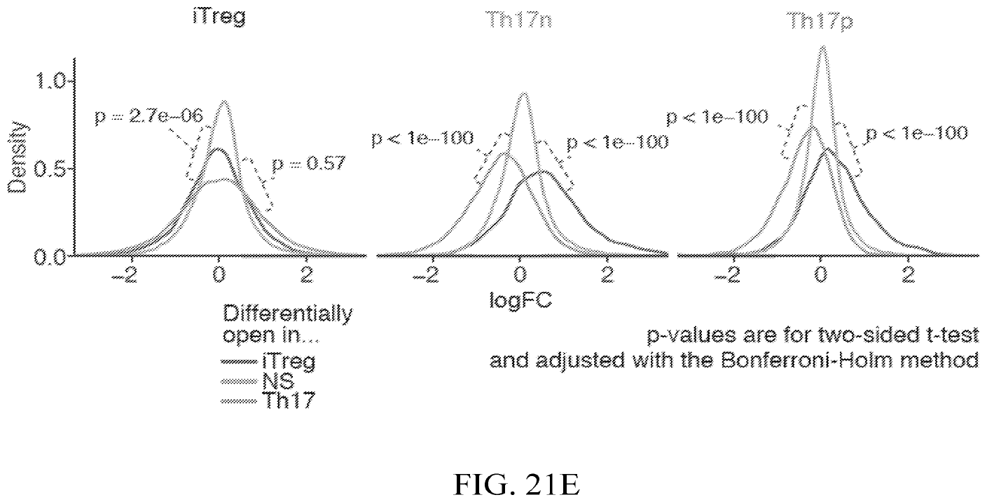

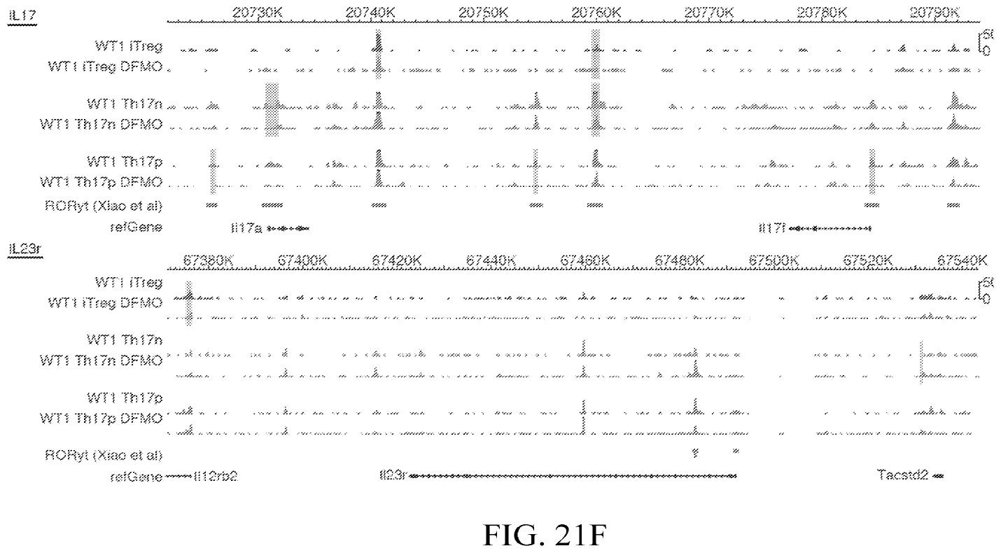

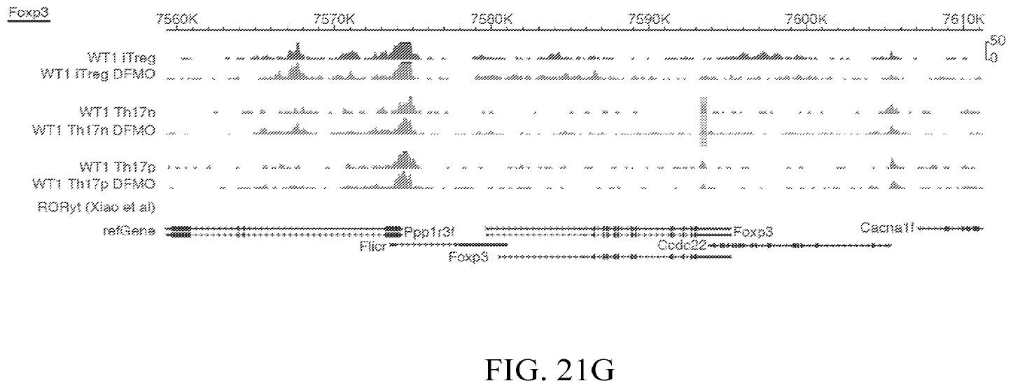

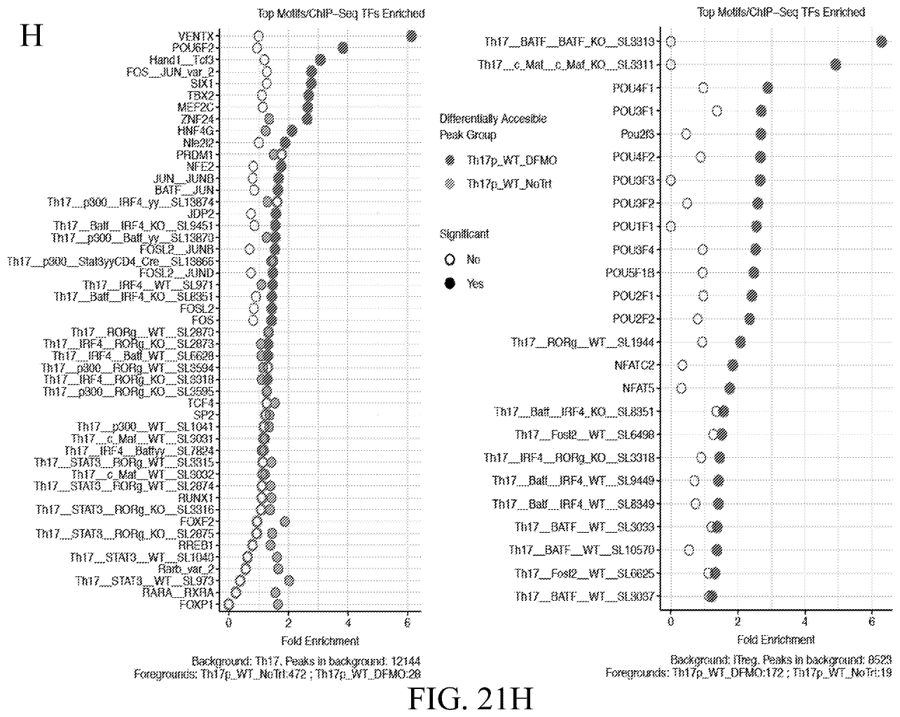

Applicants used COMPASS to characterize the metabolic heterogeneity in sorted Th17 cells and identified metabolic pathways associated with pathogenicity (FIG. 1A, 20A-B, 21A-E). Applicants developed a novel algorithm, COMPASS, which belongs to the family of Flux-Balance-Analysis (FBA) methods. The inputs to COMPASS are gene expression data (e.g., single cell RNA-Seq, bulk RNA-Seq and microarray), and a metabolic database, for example, based on the published Recon2 database (see, e.g., Thiele et al., A community-driven global reconstruction of human metabolism. Nature Biotechnology. 2013; 31:419-425; rdmio/bioc/BiGGR/man/Recon2.html; and Swainston, et al., Recon 2.2: from reconstruction to model of human metabolism. Metabolomics. 2016; 12: 109). The database is a human genome-scale metabolic reconstruction that details all known metabolic reactions occurring in humans. The specifically database includes: 1. stoichiometry of metabolic reactions; 2. associations of metabolic reactions with genes coding their respective enzymes; and 3. COMPASS runs a mathematical optimization procedure, which simulates the metabolic fluxes at a single-cell level, and produces a quantitative metabolic profile of each cell. First, COMPASS translates the unique transcriptomic profile of every single-cell into a set of cell-specific mathematical constraints and projects them onto the network. For example, the gene expression of a Th17 cell can show high expression of glucose intake (GLUTs), an intermediate glycolytic enzyme (Pkm), and pyruvate fermentation into lactate (Ldha). This is a classic glycolytic shift that occurs in pathogenic Th17 cells. Another Th17 cell can show low glucose intake and no Ldha, but expresses β-oxidation genes that break fatty-acids to generate ATP. This is a classic profile of Treg and T memory cells, and Applicants observe it in the non-pathogenic Th17 cells. Compass predicted that Th17 pathogenicity is associated with increased flux through the polyamine pathway. Specifically, COMPASS predicted polyamine activity is positively associated with Th17 pathogenicity (FIG. 1B-D). The polyamine pathway is essential for cell proliferation, regulates histone acetylation, a target in cancer, but was not previously implicated in T helper cell function. COMPASS also predicted that the glycolysis pathway is positively associated with Th17 pathogenicity (FIG. 21E).

### Example 2—the Polyamine Pathway is Alternatively Regulated by Pathogenic and Non-Pathogenic Th17 Cells

Applicants validated the association of the polyamine pathway with pathogenic Th17 cells using fluxomics and metabolomics analysis (FIG. 2A-F). FIG. 2A shows differential abundance of polyamines (shown in FIG. 2C) between pathogenic and non-pathogenic differentiated T cells. Shown are the abundance of the indicated polyamines in the cells and the media. The media abundance can be subtracted from the total abundance to determine the abundance in the cell. Specifically, acetyl spermidine and acetyl putrescine were much higher in pathogenic differentiated T cells than in non-pathogenic differentiated T cells. These polyamines are the products of the enzyme SAT1 (FIG. 2C). FIGS. 2B, E, and F show fluxomics using C13 labeled precursors to the polyamine pathways. These results suggested alternative usage of the polyamine pathway by pathogenic and non-pathogenic Th17 cells. Pathogenic Th17 cells appear to exclusively produce acetyl spermidine (FIG. 2B). Pathogenic Th17 cells turn arginine into L-citruline, producing more NO in the process, and polyamines (FIG. 2E). Non-Pathogenic Th17 cells turn L-citruline into Arginine and creatinine (FIG. 2F). FIG. 2D shows that untargeted metabolomics using liquid chromatography/mass spectrometry (LC/MS) identified several metabolites related to polyamine pathway that are alternatively expressed in pathogenic as compared to non-pathogenic Th17 cells.

### Example 3—Polyamines and a Polyamine Analogue (DFMO) can Interfere with Th17 Cell Differentiation

Inhibition of the polyamine pathway using 2-(difluoromethyl)ornithine (DFMO) (FIG. 3A) alters Th17 cell function (FIG. 3B,C), promotes Tregs (FIG. 3E, bottom), delays EAE onset (FIG. 3E, top), and decreases proliferation of immune cells after immunization with MOG in a MOG assay (FIG. 3F). FIG. 3D shows that the addition of putrescine rescues the effect of DFMO. FIG. 311 shows that DFMO suppresses IL-17 expression, but not Rorgt in both pathogenic and non-pathogenic Th17 cells. FIG. 3G shows that addition of polyamines can interfere with Th17 differentiation. FIG. 3I shows that addition of DFMO alters production of cytokines in pathogenic Th17 cells and non-pathogenic Th17 cells. For example, differences are seen in IFNg between pathogenic and non-pathogenic Th17 cells. FIG. 3J shows an increase in FoxP3 CD4 T cells (Tregs) in nonpathogenic Th17 cells after DFMO treatment. Thus, DFMO can be used to increase a suppressive immune environment. FIG. 3K shows that the addition of putrescine rescues the effect of DFMO in pathogenic and non-pathogenic Th17 cells. FIG. 3L shows that the addition of putrescine rescues the increase in FoxP3 CD4 T cells (Tregs) in nonpathogenic Th17 cells after DFMO treatment.

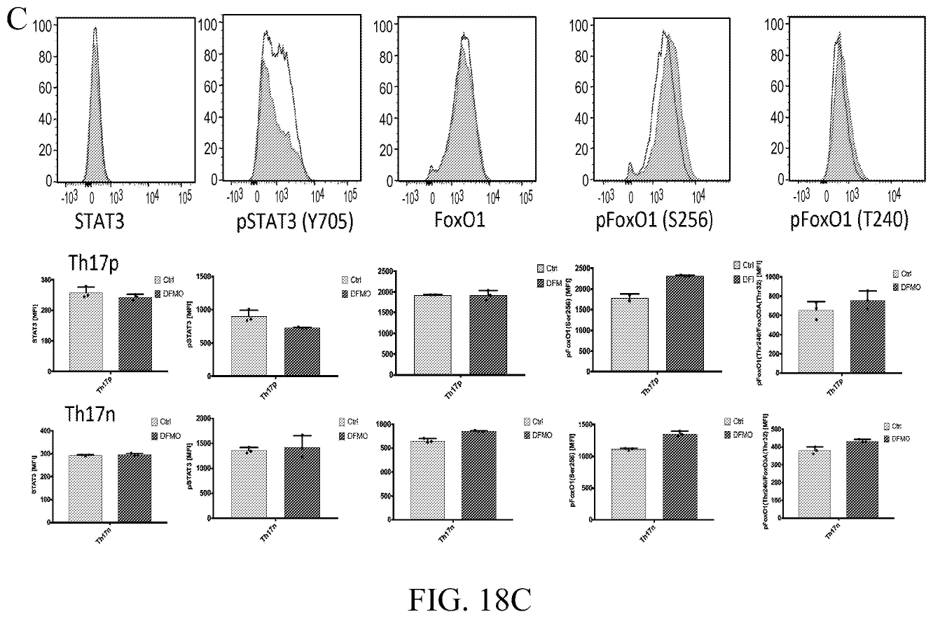

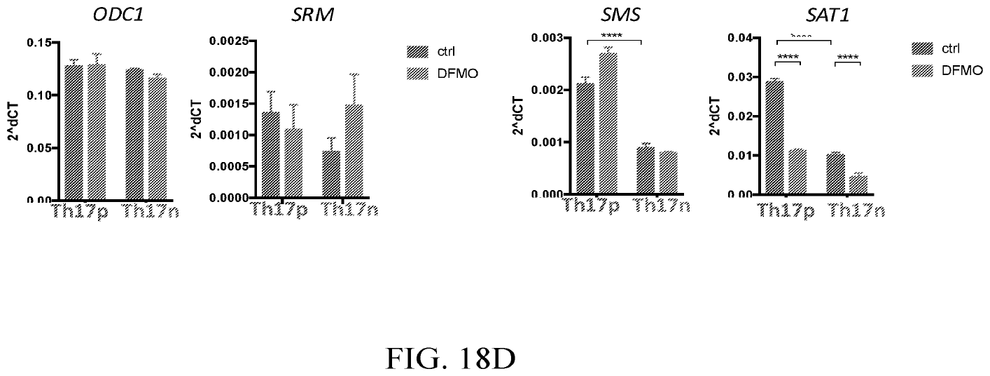

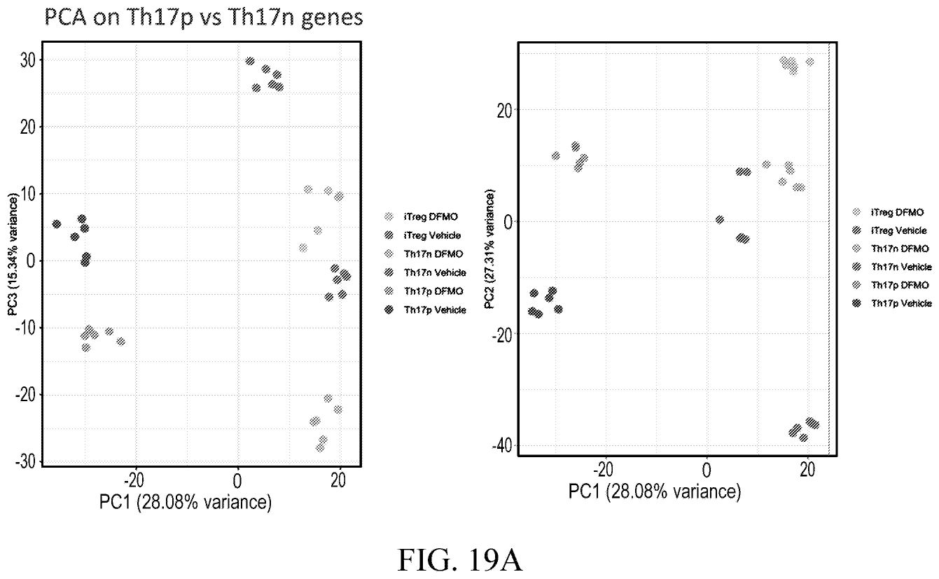

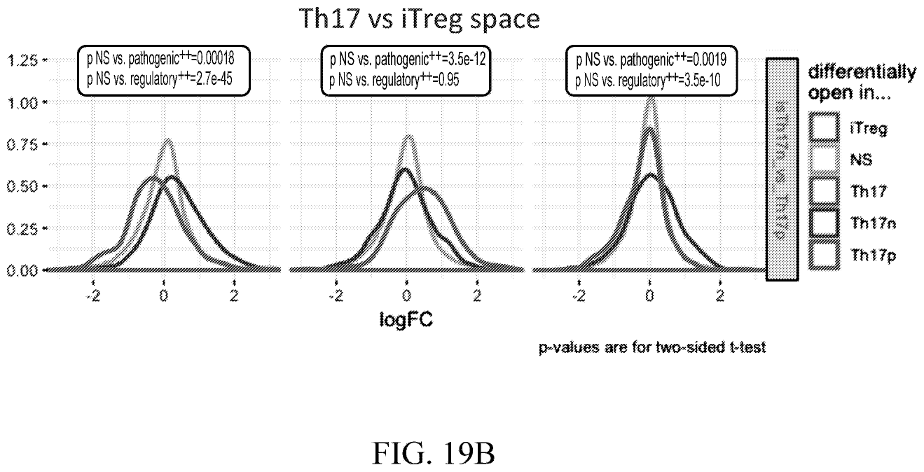

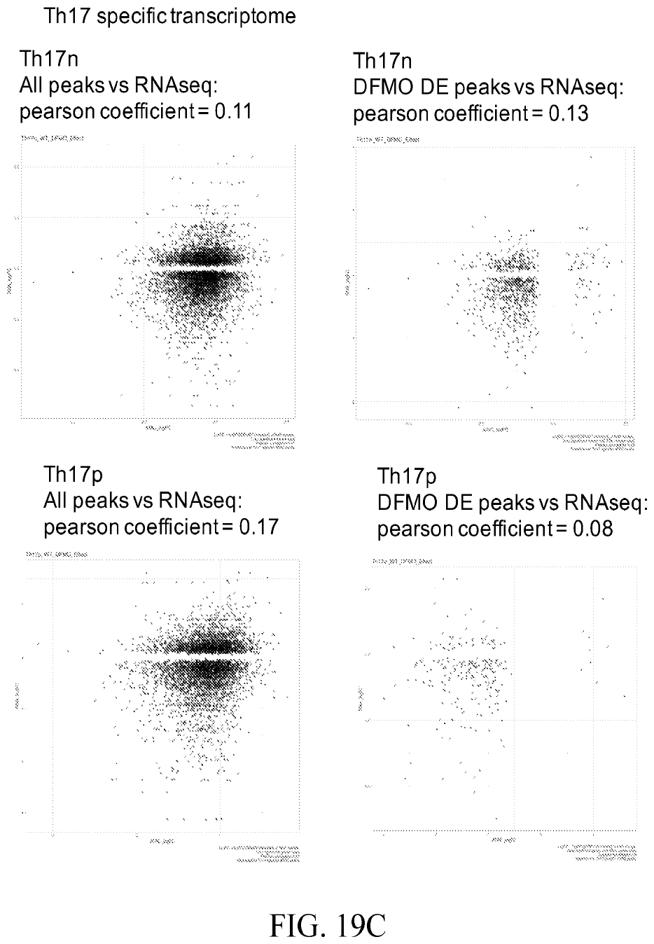

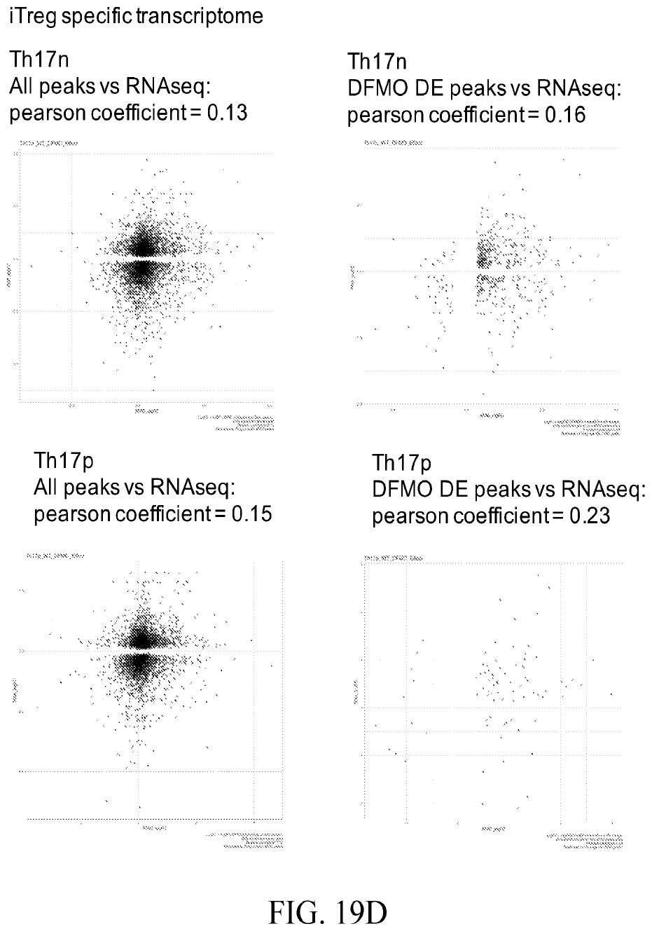

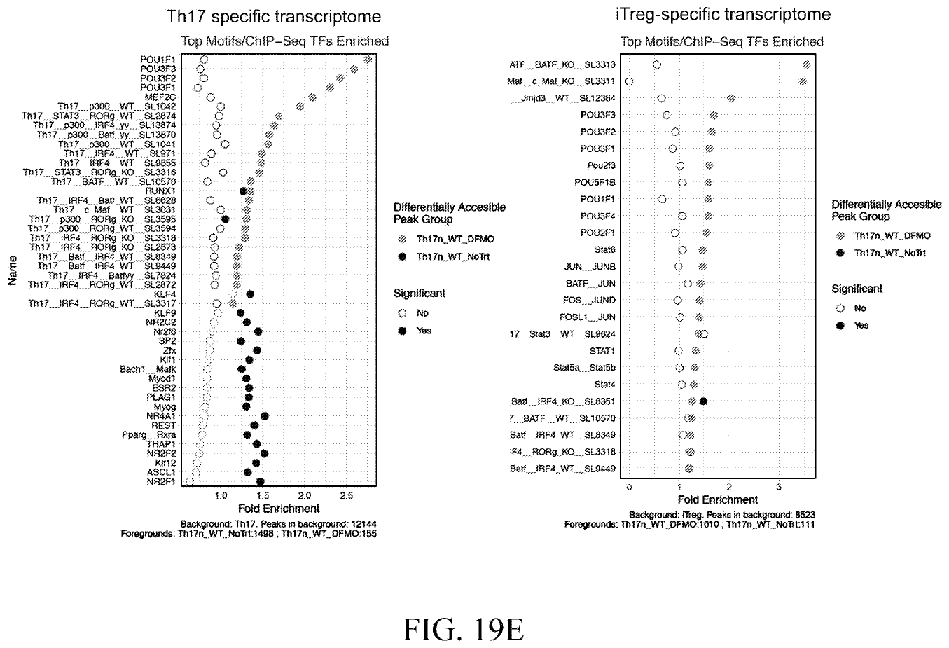

FIG. 4 shows that inhibition of the polyamine pathway transitions Th17 cells into a Treg-like transcriptome. Treatment of Th17 and iTreg cells with DFMO shift the cells towards Treg gene expression (PCI) (FIGS. 4A and 19A). As DFMO blocks the polyamine pathway upstream of SAT1, knockout of SAT1 does not affect the results. DFMO treatment on Th17 cells shift Th17 specific genes down and shift Treg specific genes up in Th17 non-pathogenic and pathogenic T cells (FIG. 4B,C). Genes shared between Th17 and Treg cells do not change (FIG. 4B). FIG. 4D shows decrease in expression of IL17A and IL17F, and increase in expression of Foxp3 in non-pathogenic and pathogenic Th17 cells after DFMO treatment. FIG. 4E shows that DFMO also alters chromatin associated genes in pathogenic Th17 cells. FIG. 4F shows that DFMO alters chromatin accessibility of Th17 and iTreg ATAC-seq peaks in non-pathogenic Th17 cells and pathogenic Th17 cells. FIG. 4G shows that DFMO affects chromatin accessibility and the associated gene expression in non-pathogenic Th17 cells and pathogenic Th17 cells.

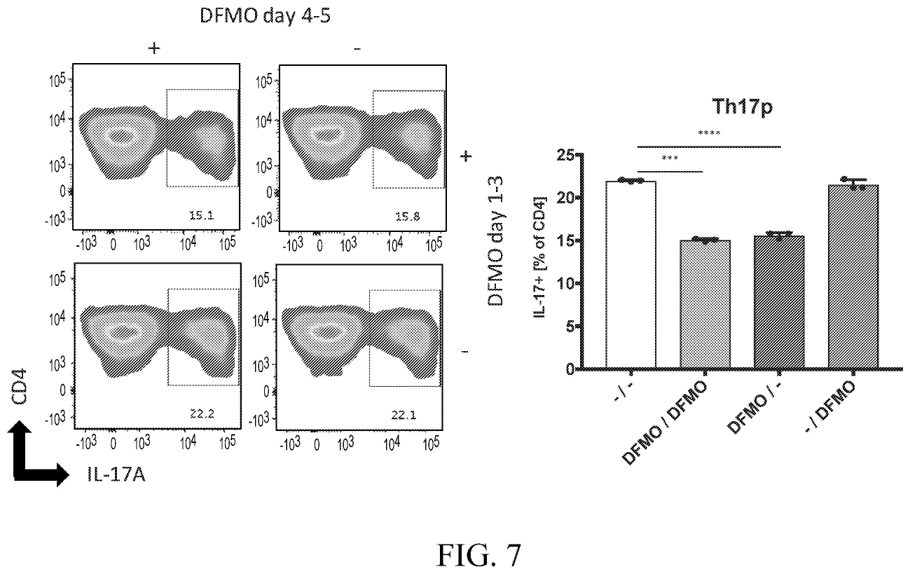

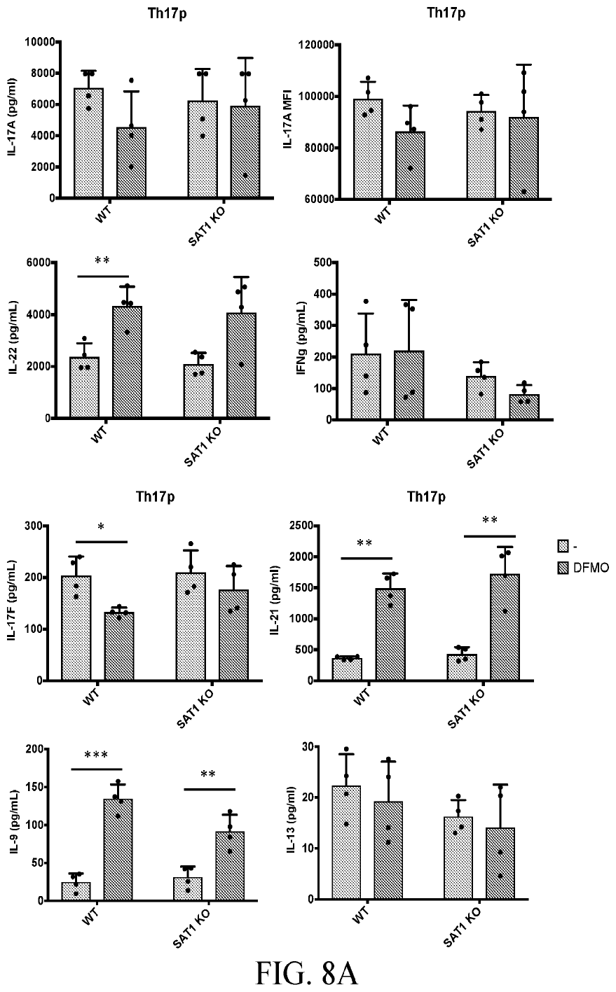

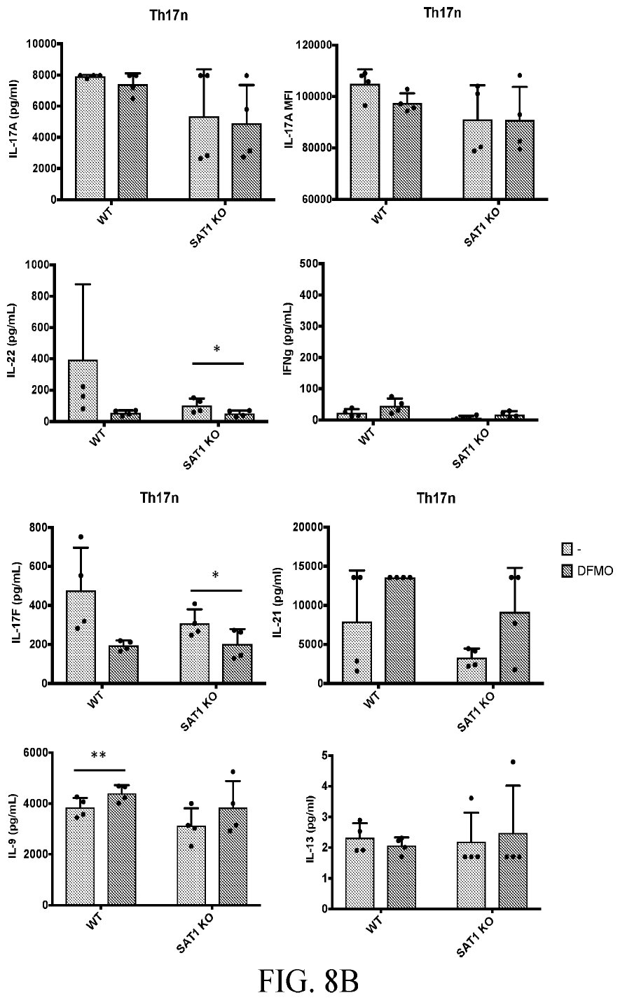

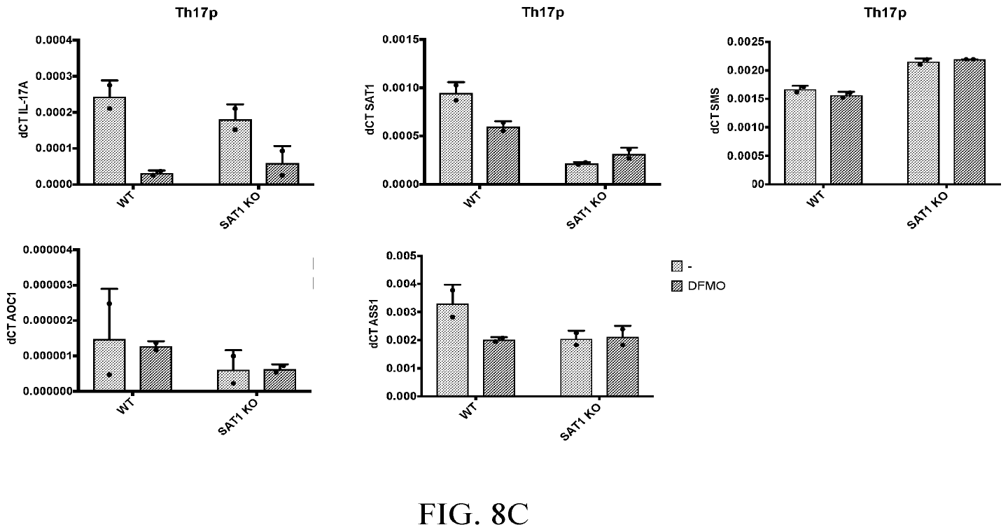

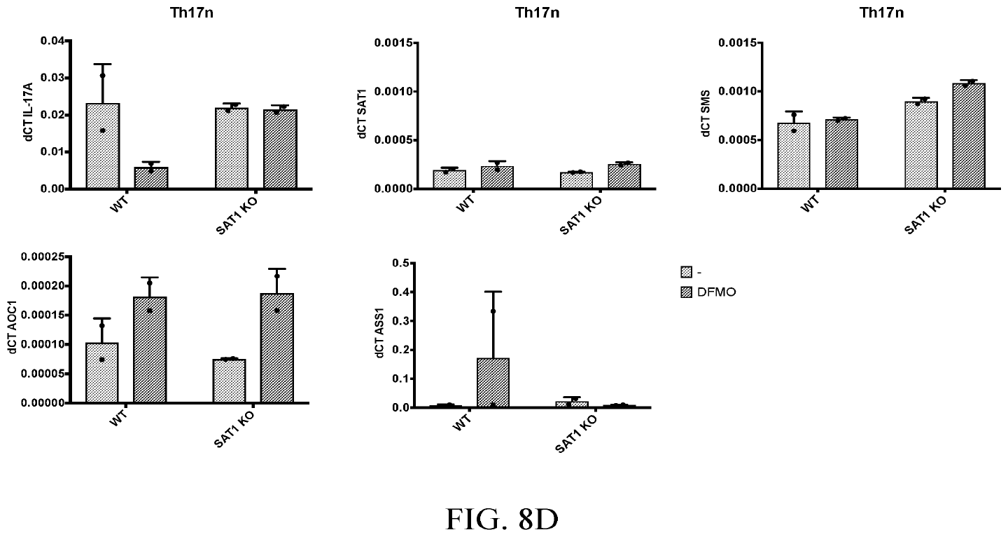

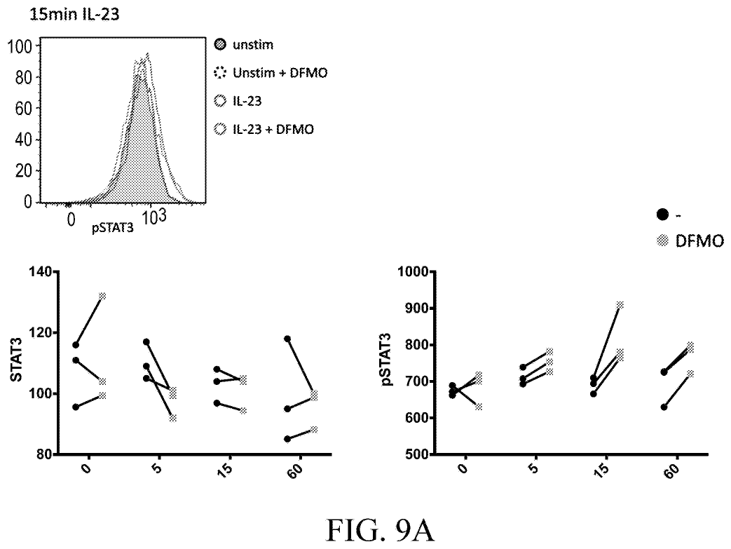

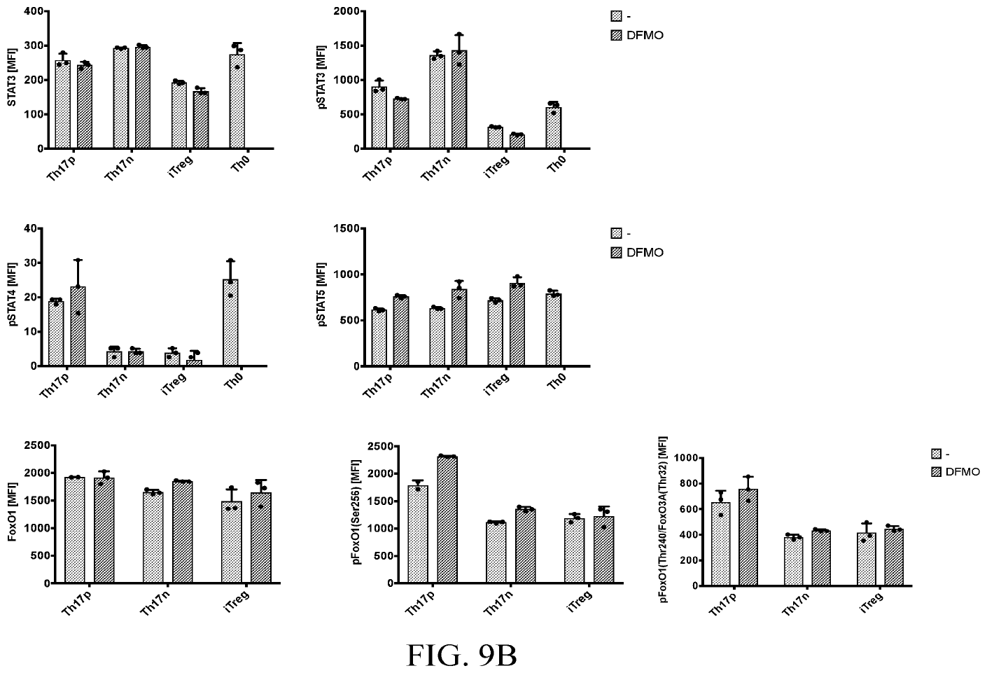

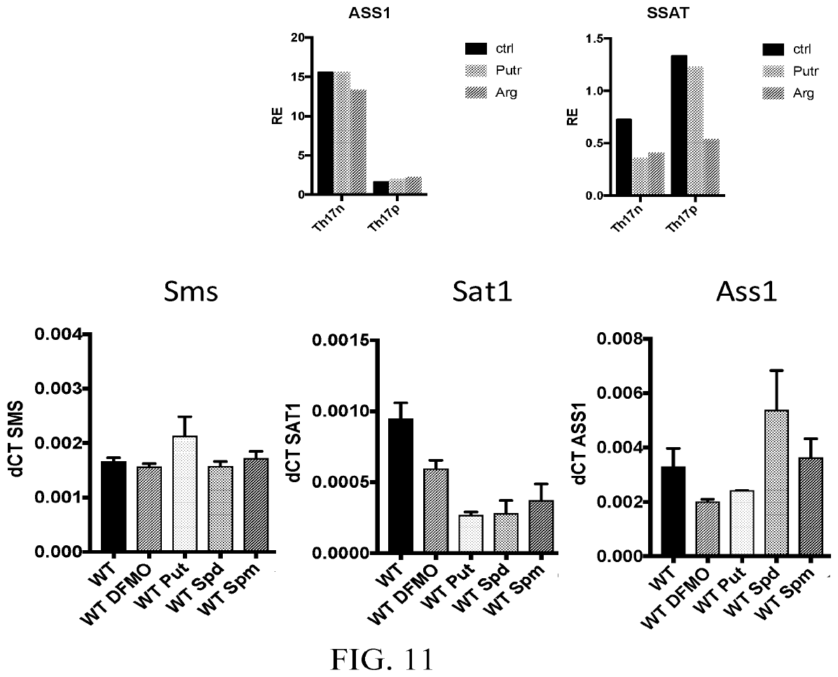

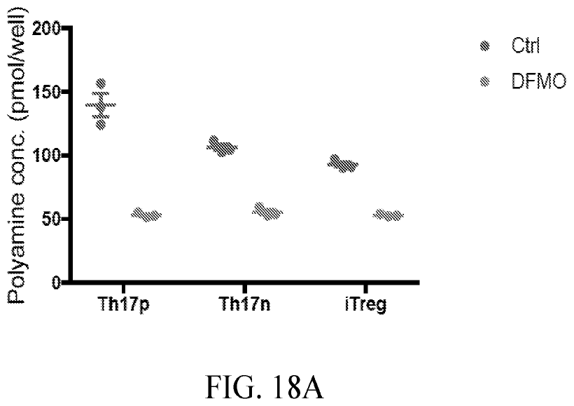

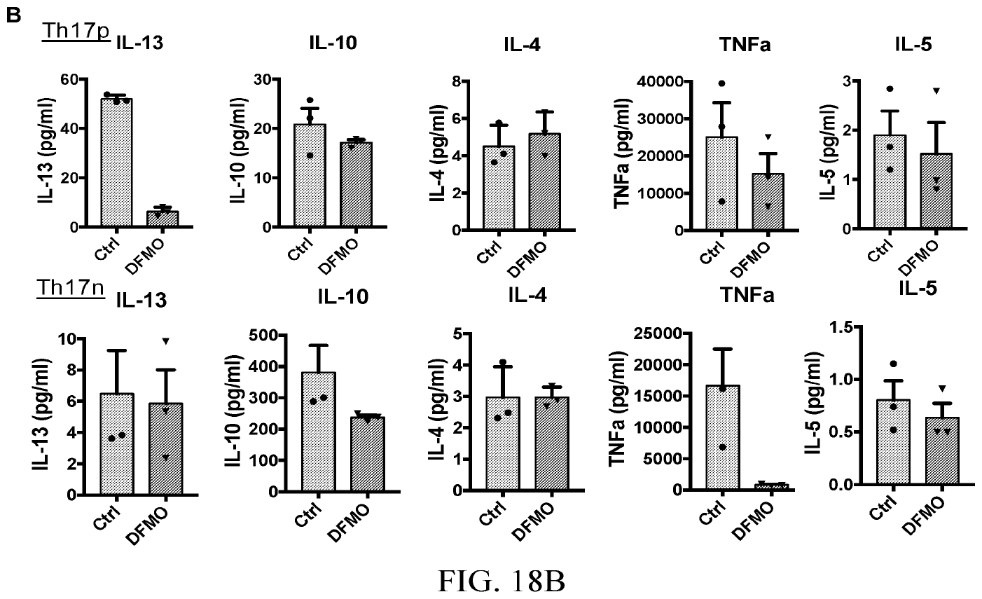

Applicants show that suppression of IL-17 by DFMO is dependent on the timing of DFMO treatment (FIG. 7). When DFMO is administered at both days 1-3 and days 4-5 or at days 1-3 only, IL-17+ cells decrease, whereas there is no change when DFMO is administered at 4-5 days only. Thus, cells treated with DFMO at the time of differentiation, but not during expansion phase of Th17 cells showed the decrease in IL-17+ cells. DFMO also promoted IL-21, IL-22 and IL9 expression in Th17 cells, specifically pathogenic Th17 cells (FIG. 8A,B). Altered IL-17 and SAT1 expression in Th17 cells in response to DFMO, as well as changes in polyamine enzymes was also observed using quantitative PCR (FIG. 8C,D). DFMO treatment did not alter pStat3 expression, a transcription factor essential for Th17 differentiation (FIG. 9). FIG. 11 also shows that DFMO and polyamines alter enzymes of the polyamine pathway. DFMO treatment specifically suppresses Sat1 and Ass1 (FIG. 11). DFMO causes a decrease in polyamine concentration in iTregs, non-pathogenic Th17 cells, and pathogenic Th17 cells (FIG. 18A). FIG. 18B shows production of cytokines is altered in pathogenic and non-pathogenic Th17 cells after DFMO treatment. The alarmins, especially IL-13 in pathogenic Th17 cells is decreased. FIG. 18C shows that the indicated phosphorylated transcription factors are altered in pathogenic and non-pathogenic Th17 cells after DFMO treatment.

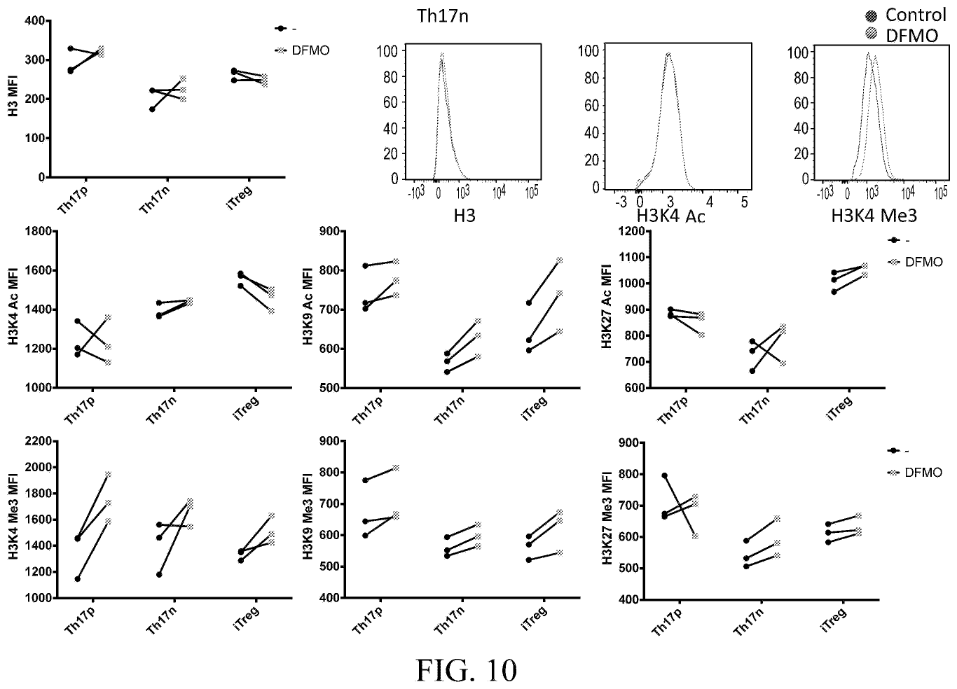

FIG. 5 shows that DFMO reduces accessibility in regions accessible in Th17 cells, but inaccessible in Treg cells. Thus, the polyamine pathway may affect gene expression by altering chromatin structure. DFMO promoted H3K4, H3K27, H3K9 trimethylation in Th17 cells (FIG. 10). FIG. 19B further shows chromatin accessibility of non-pathogenic Th17 and pathogenic Th17 genes.

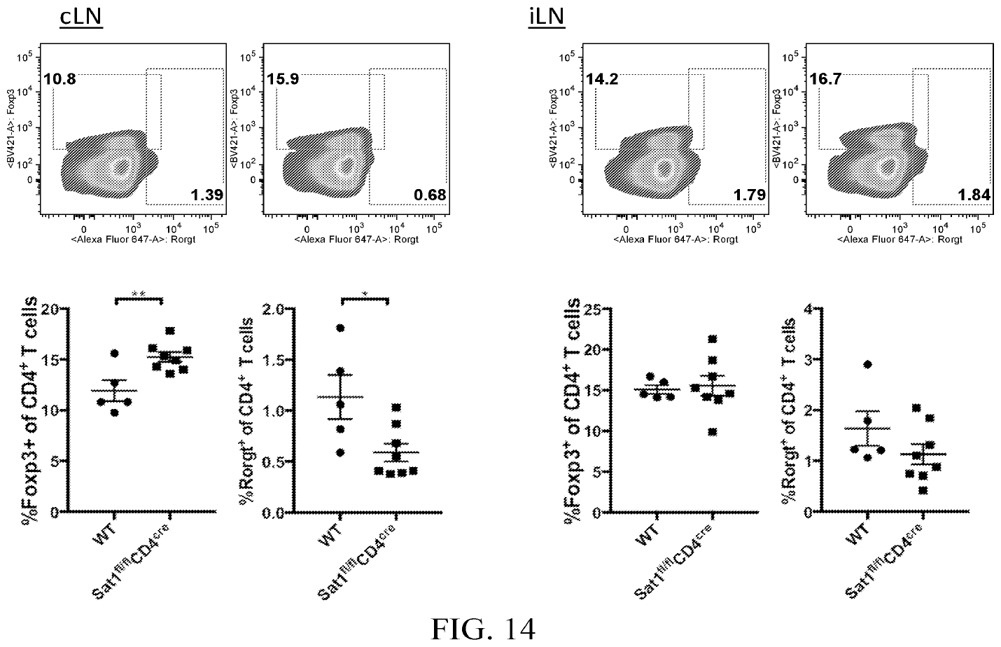

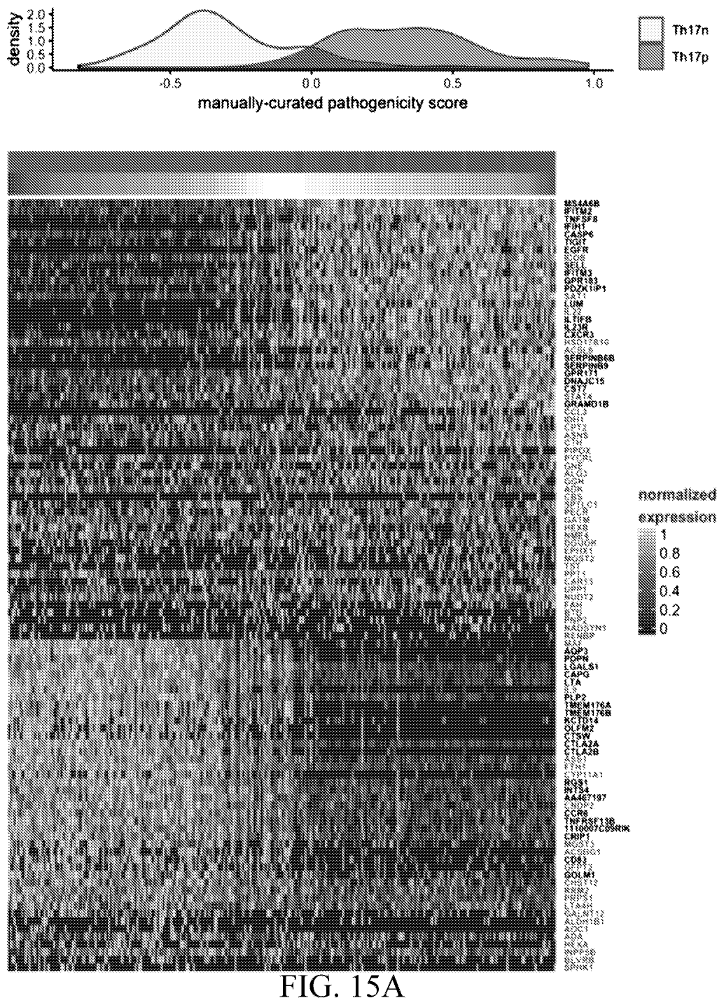

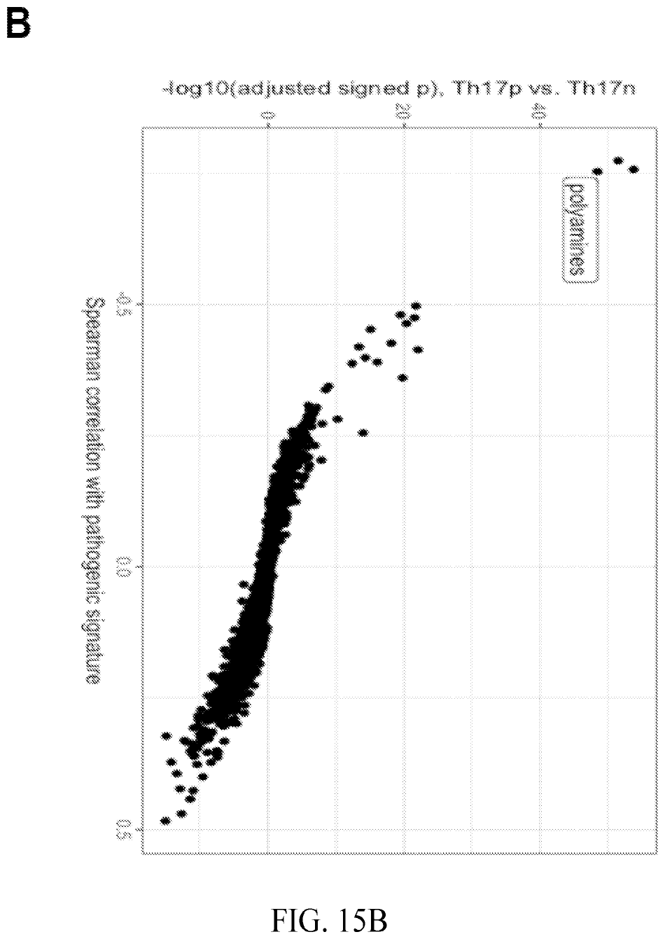

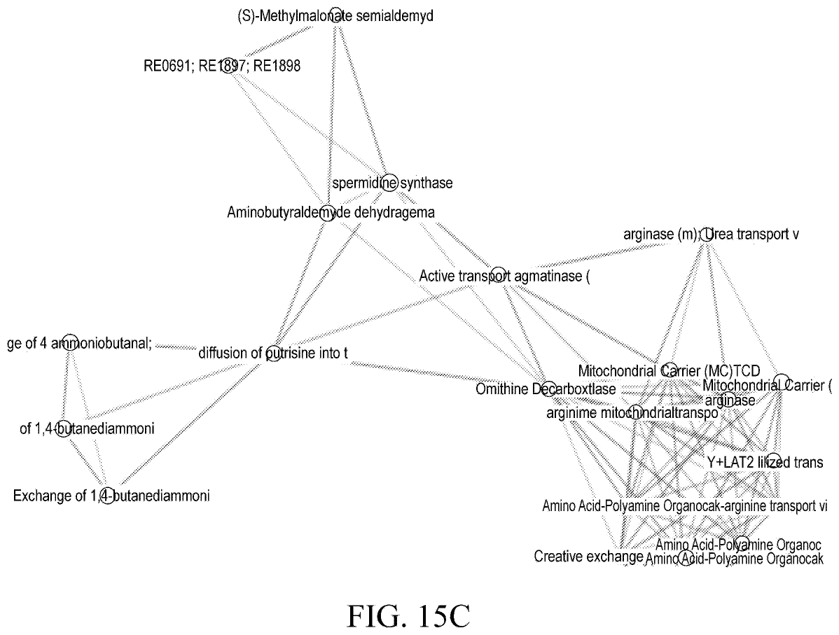

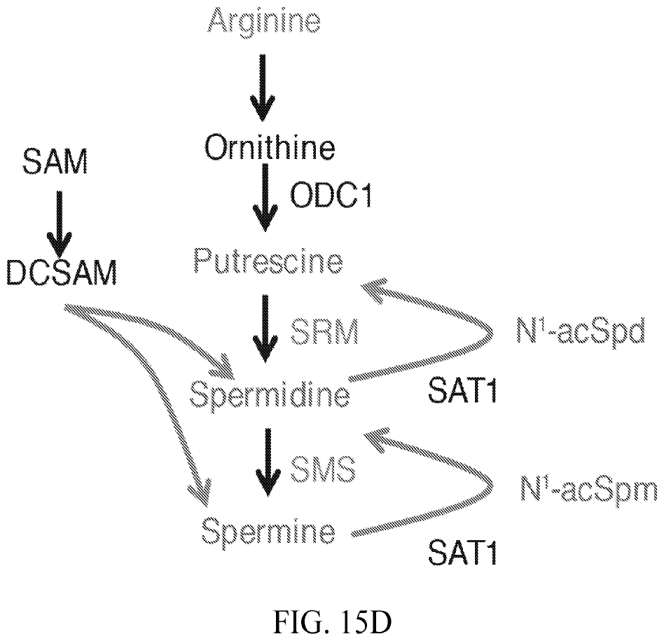

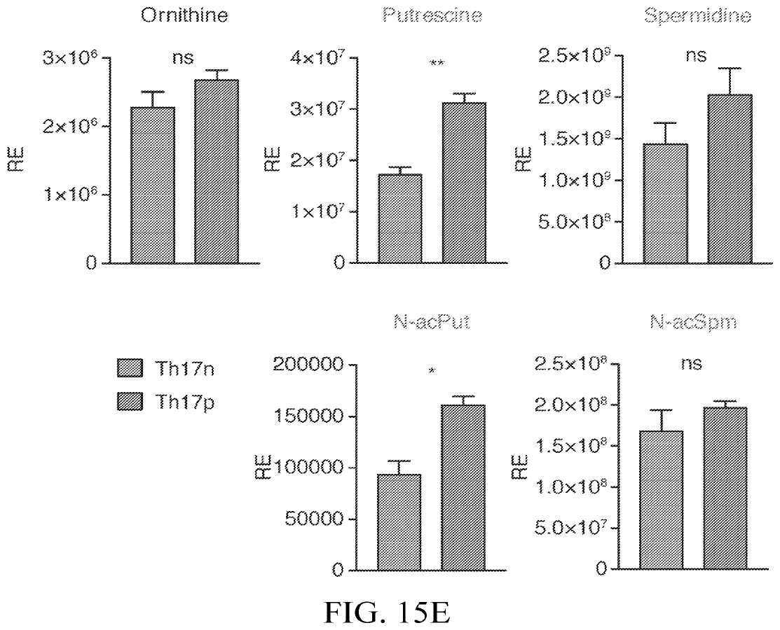

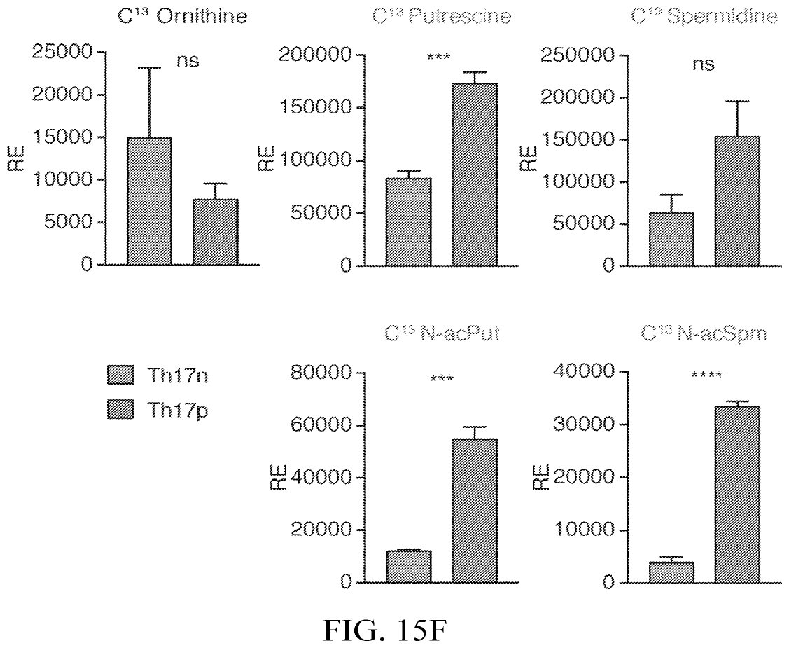

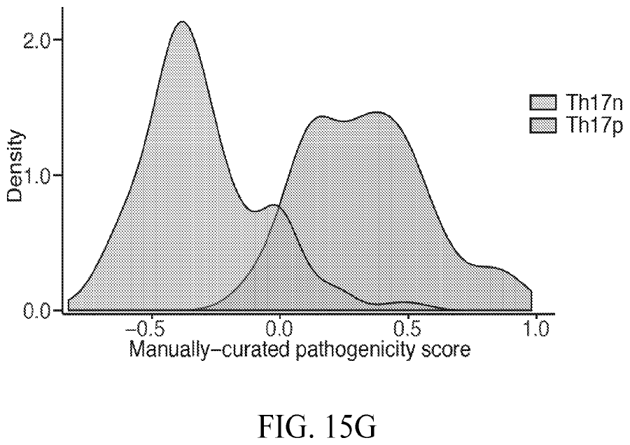

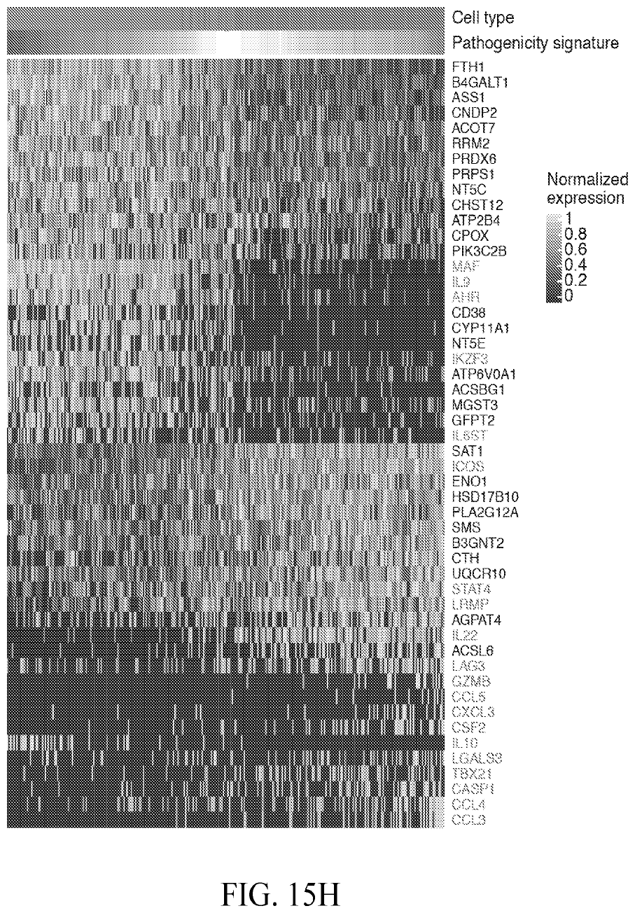

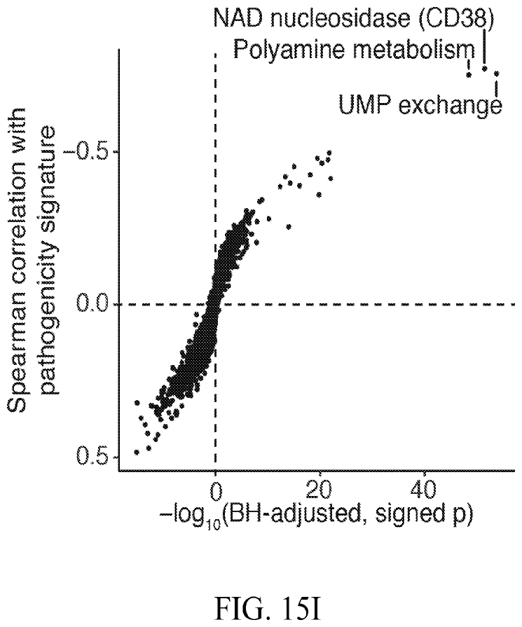

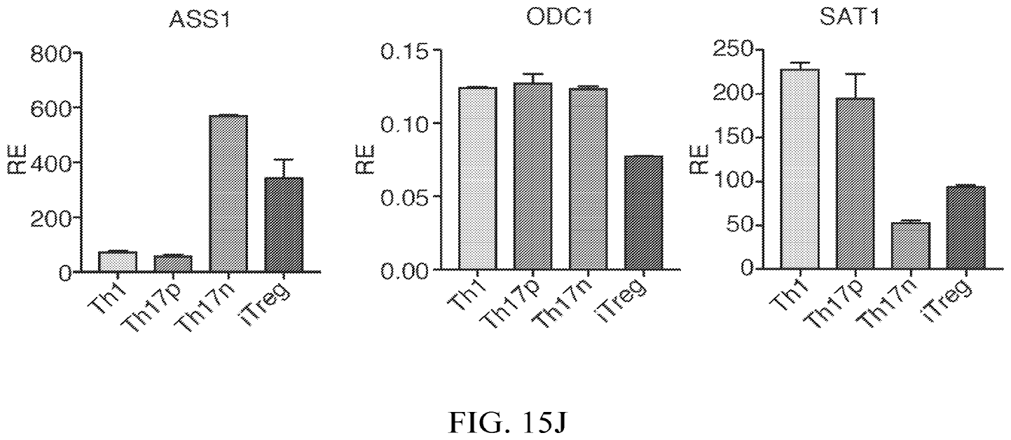

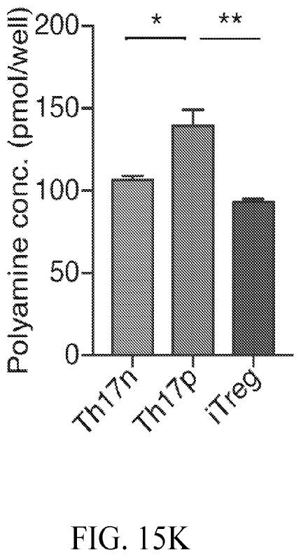

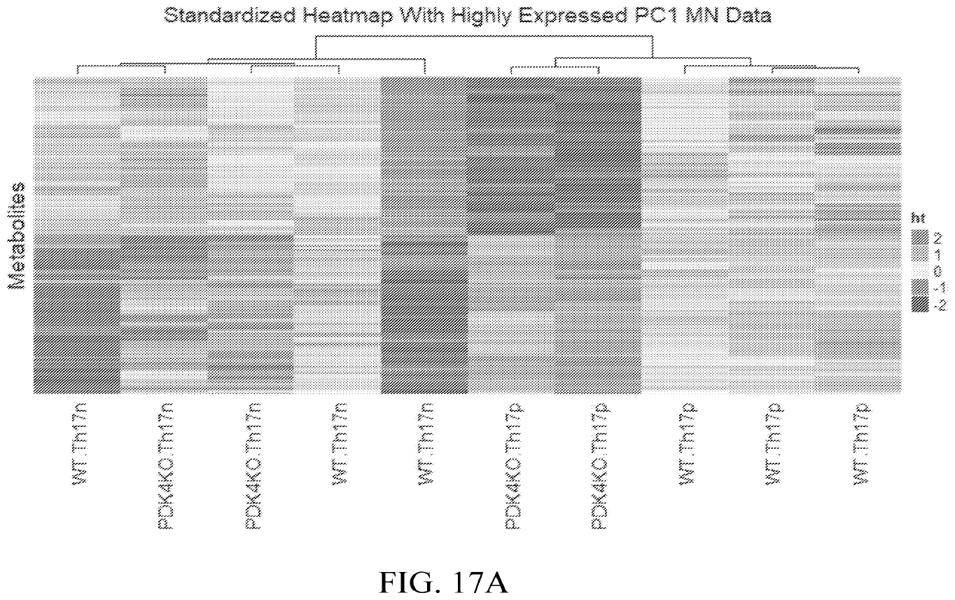

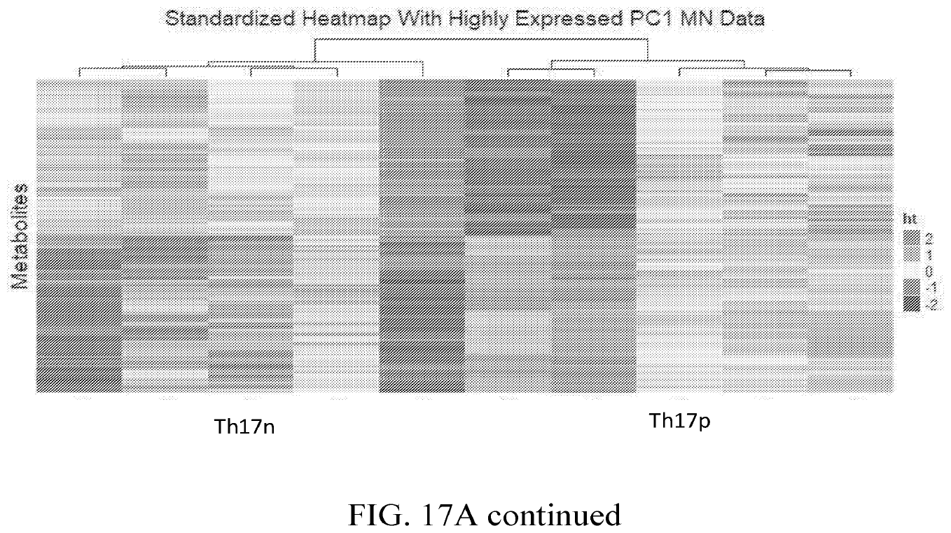

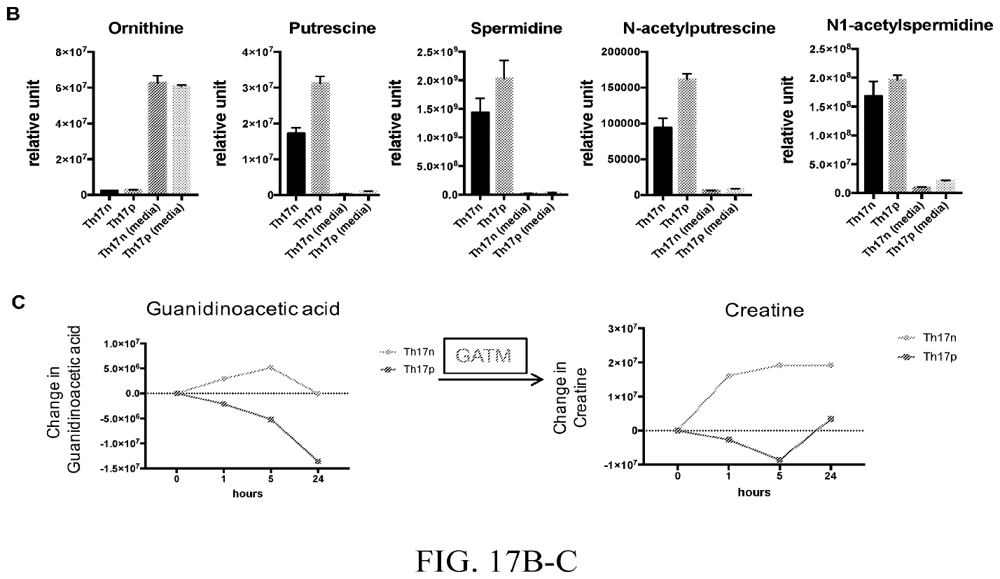

FIG. 15A further shows gene expression in pathogenic and non-pathogenic Th17 cells. FIGS. 15B and 15C further show that polyamines correlate with the pathogenic signature. FIG. 15E shows differential polyamine expression in Th17 cells. FIG. 15F shows that Th17 cells differentially synthesize polyamines. FIG. 17 shows differential expression of metabolites in the indicated Th17 cells. Metabolites are different between non-pathogenic and pathogenic Th17 cells.

### Example 4—Perturbation of Sat1 Modulates Th17 Function

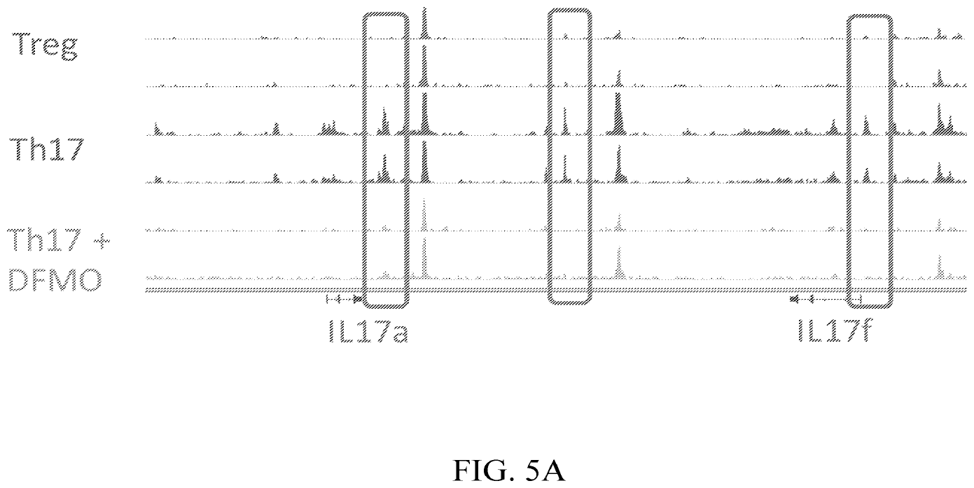

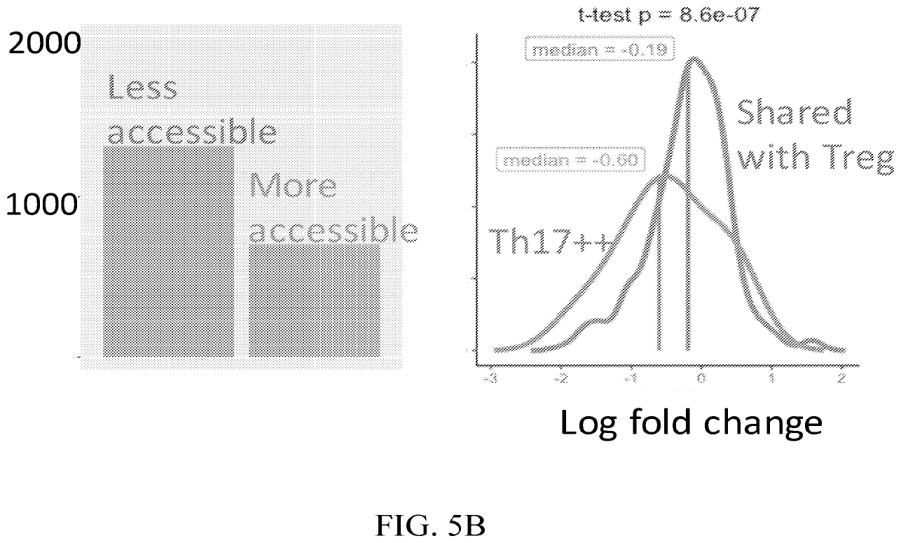

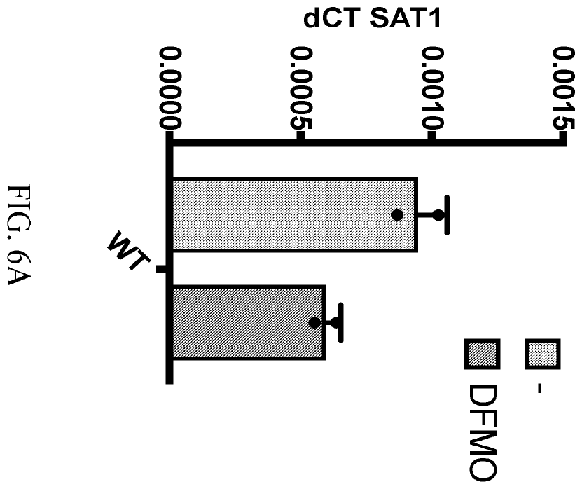

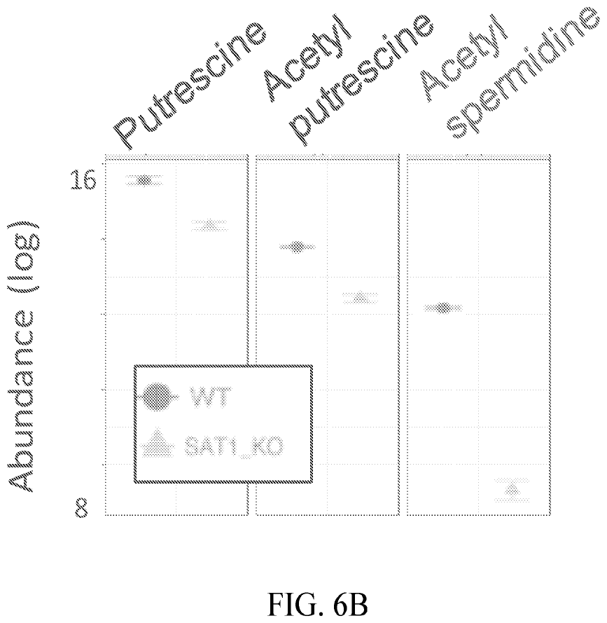

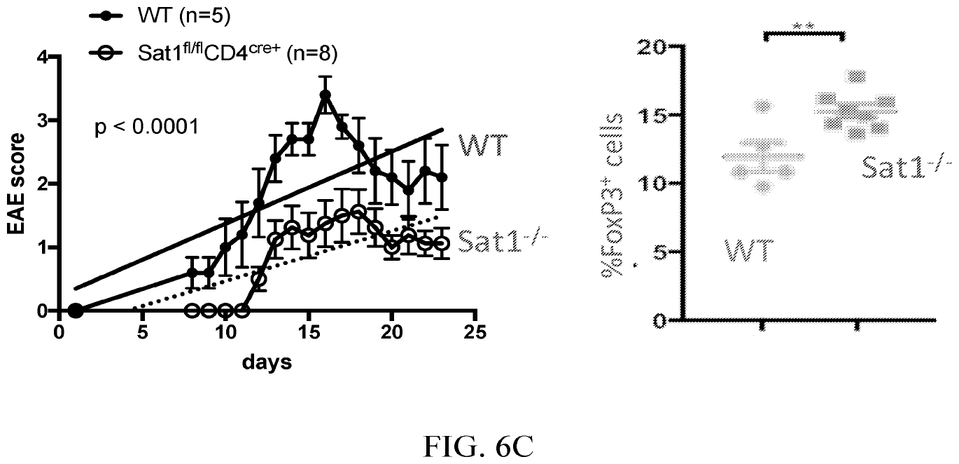

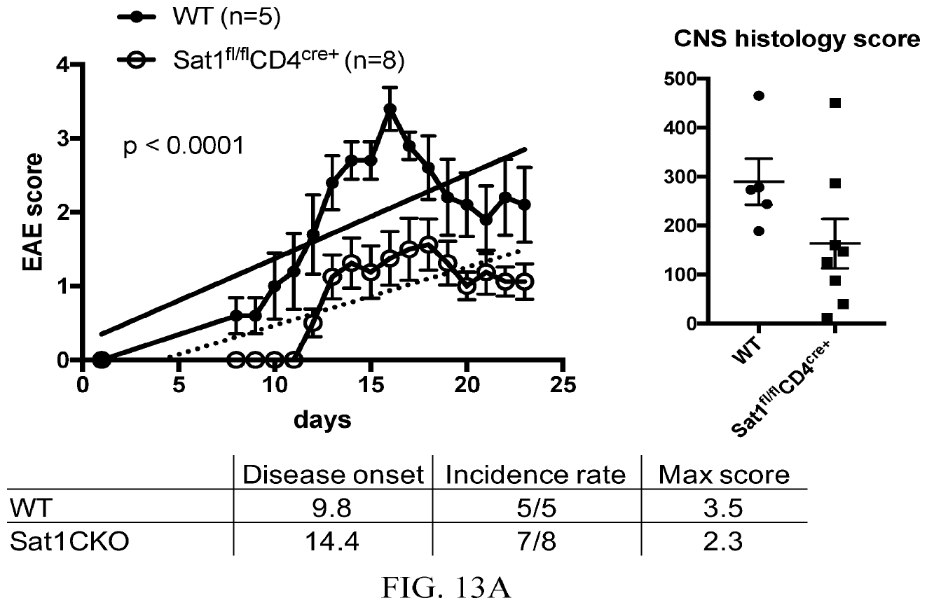

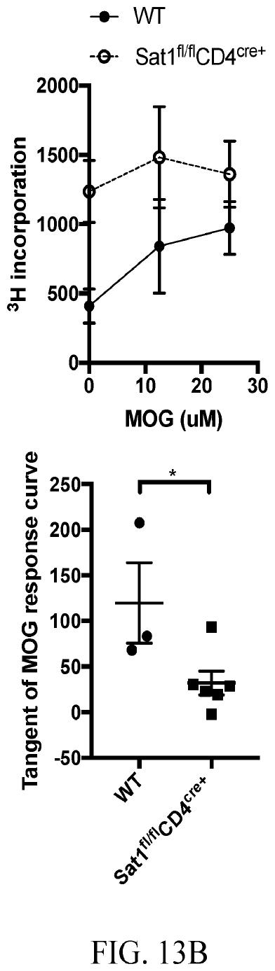

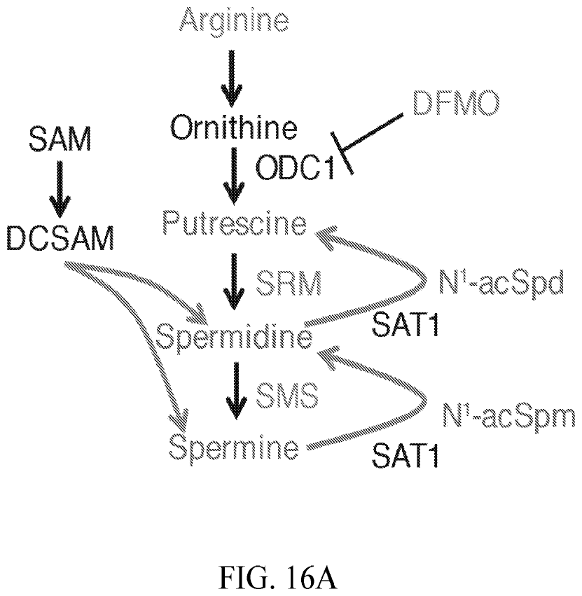

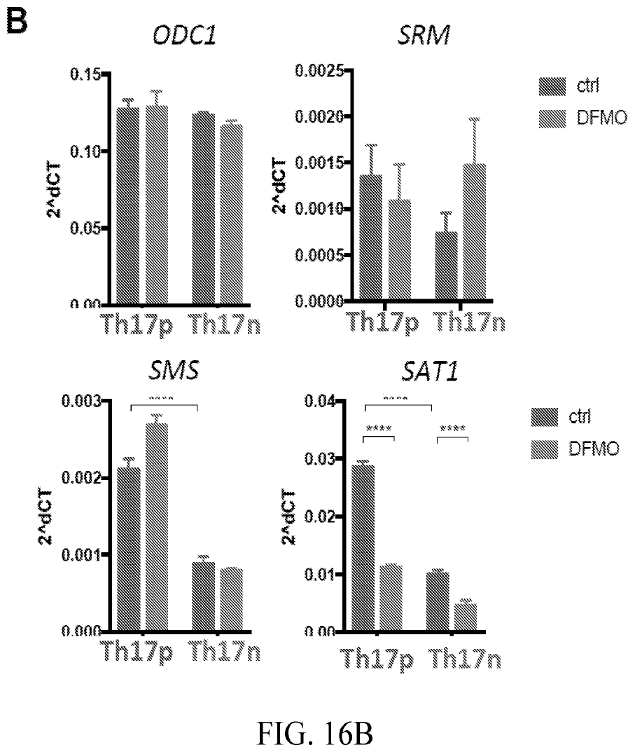

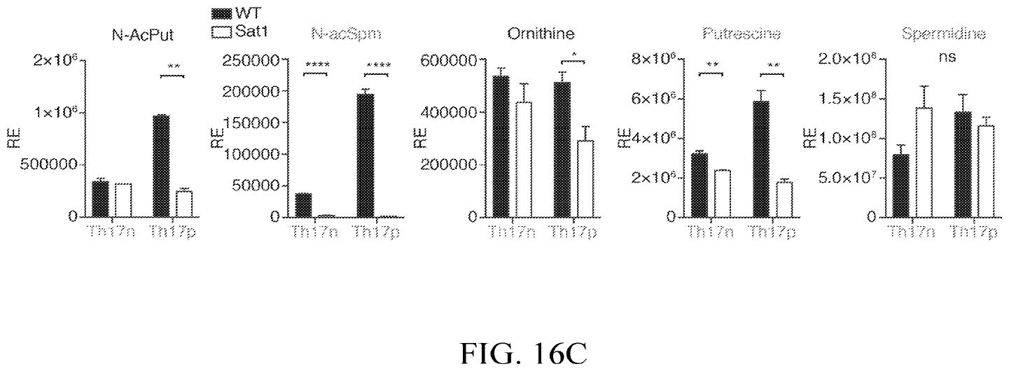

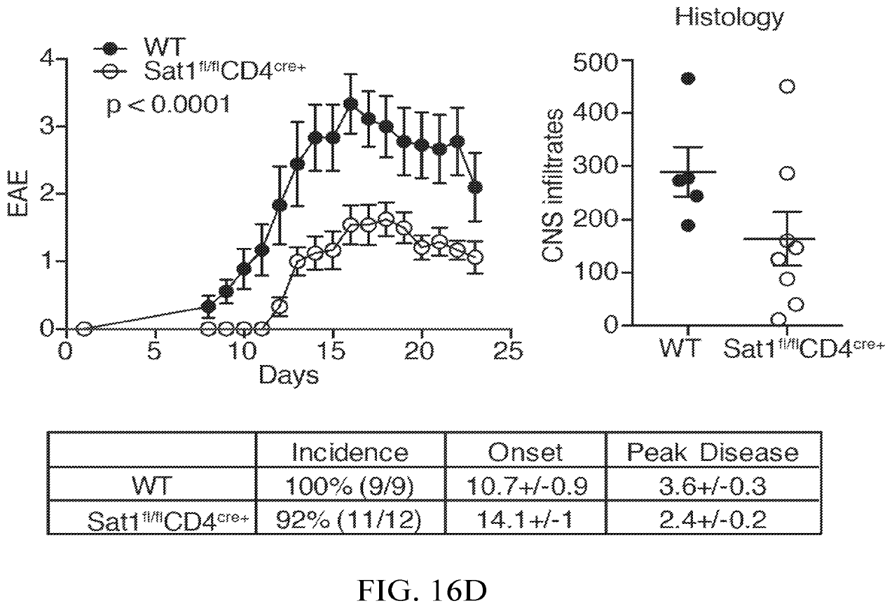

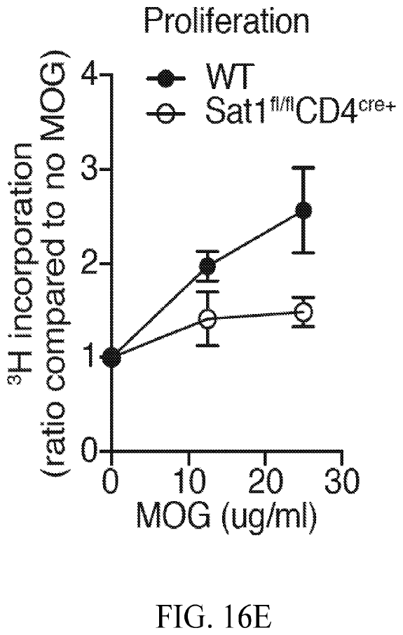

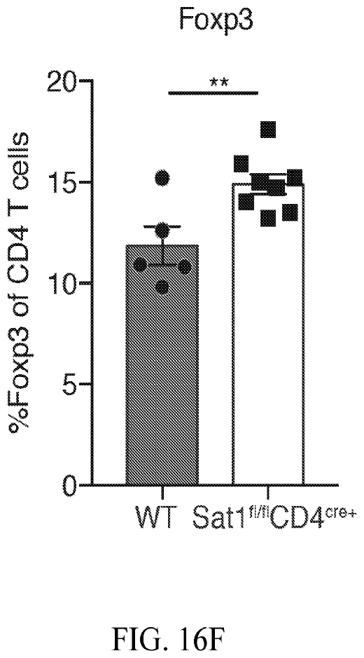

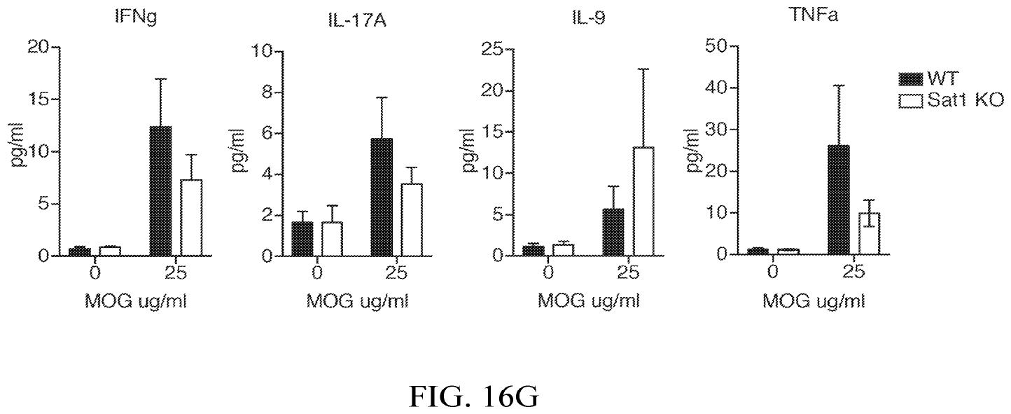

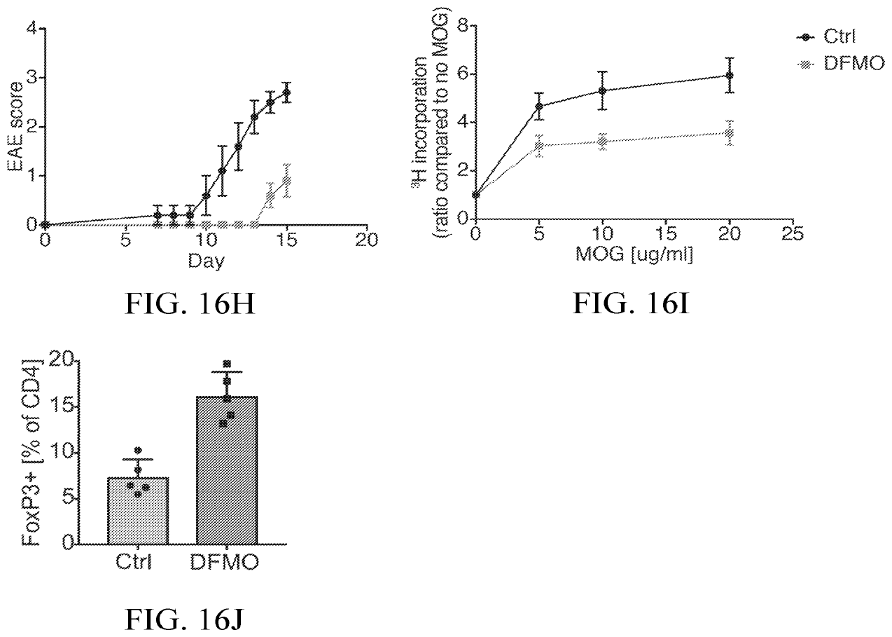

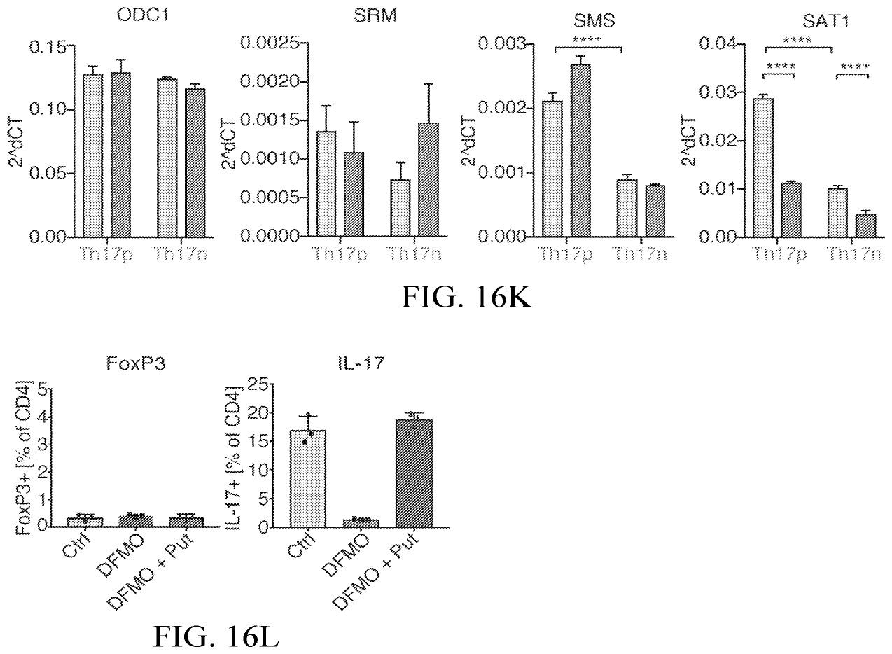

Applicants Treatment of T cells with DFMO decreases expression of SAT1 (FIG. 6A). Conditional deletion of SAT1 in T cells decreases the abundance of acetyl spermidine (FIG. 6B, 12A). FIG. 16B shows that DFMO differentially affects expression of polyamine enzymes, especially decreased expression of SAT1 in pathogenic Th17 cells. FIG. 16C shows that Sat knockout differentially affects polyamine expression. Conditional deletion of SAT1 in T cells alleviated EAE severity in a mouse model and promoted frequency of Tregs (FoxP3+) (FIG. 6C, 13A 16D,F). FIGS. 13B, 16E and 16G show the immune response to MOG in WT and SAT1 conditional deletion mice.

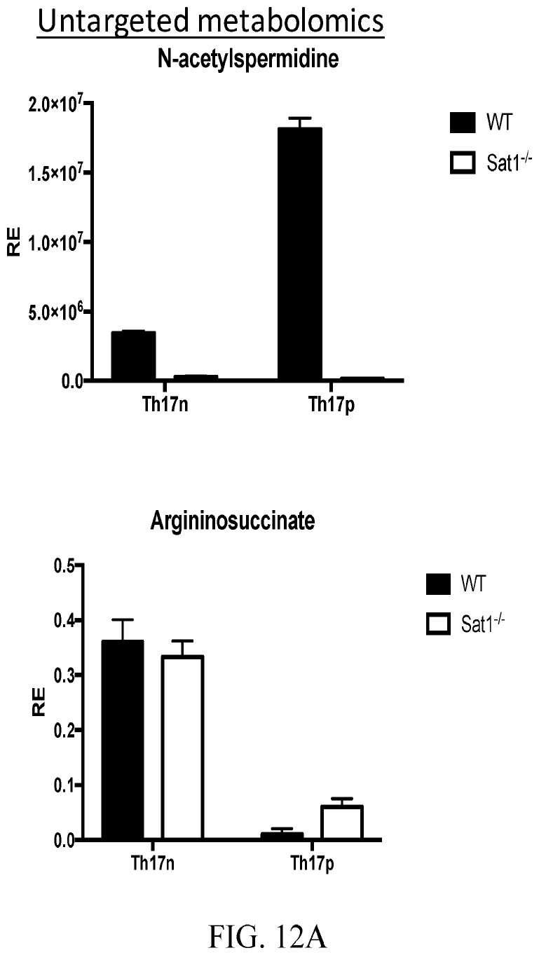

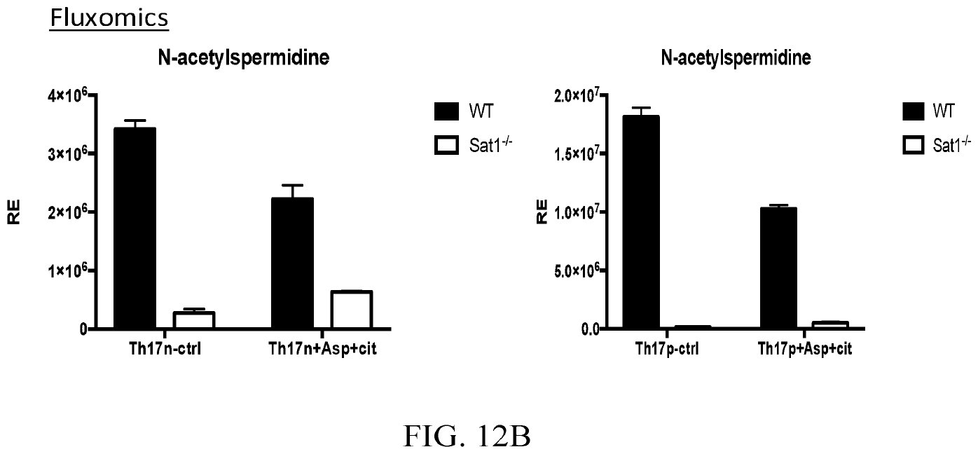

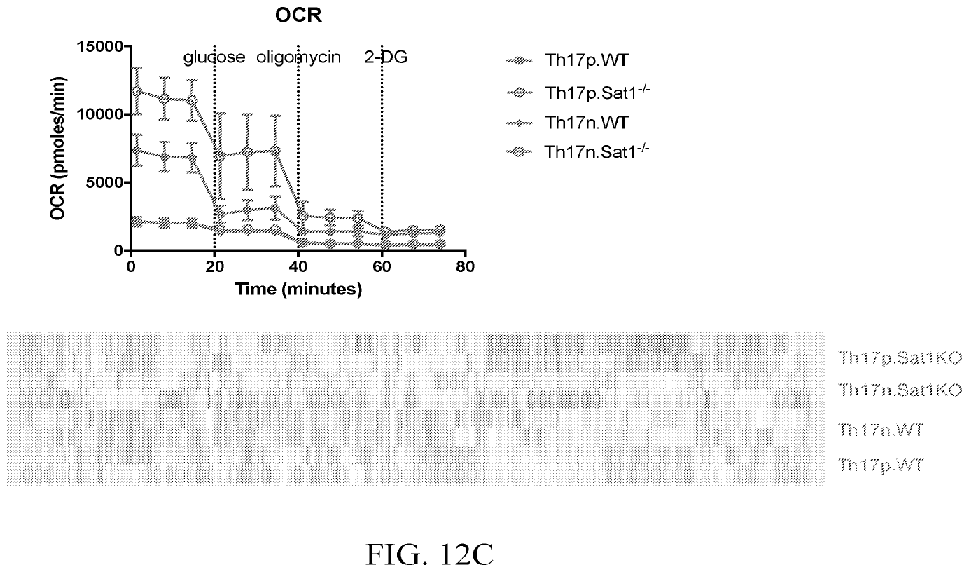

FIG. 12B shows the relative expression of N-acetylspermidine in pathogenic and non-pathogenic Th17 cells from both wild type and SAT1 KO mice and treated with the indicated polyamines. FIG. 12C shows a cell metabolism assay (see, e.g., Na et al., Mol Cell Proteomics. 2015 Oct; 14(10): 2722-2732) and differentially expressed genes. FIG. 14 shows that conditional deletion of SAT1 in T cells increases cells expressing a Treg marker (FoxP3+) and decreases Rorgt+ cells.

Thus, Applicants found that perturbation of Sat1 partially mimics has an additive effect with DFMO on Th17 cell function, and alleviates EAE.

### Example 5—the Polyamine Pathway is a Node in Metabolic Circuitry that Restricts Th17 Cell Epigenome and Proinflammatory Function

Cellular metabolism is a mediator and modulator of immune cell differentiation and function. Applicants previously identified lipid biosynthesis as a key regulator of helper TH17 cell function by altering transcriptional activity of Rorgt [1], providing proof of concept that metabolic processes can be directly involved in gene regulation and balancing proinflammatory and regulatory states of T cells. A full appreciation of metabolic circuitry and its connection with immune cell function is limited by available technology that typically investigates the average metabolic state of a large population of cells. Applicants developed a novel algorithm (COMPASS) that allows prediction of metabolic fluxes of cells using transcriptome data at the single cell level, allowing comprehensive profiling of how metabolic pathways are interconnected within a cell. Combining this novel tool, metabolomics and functional biology, Applicants investigated Th17 cells at different functional state in the context of EAE and identified the polyamine pathway as a modulator of epigenetic landscape and function of proinflammatory Th17 cells in autoimmune responses.

Polyamines are polycations including putrescine (Put), spermidine (Spd) and spermine (Spm) mainly synthesized from ornithine/methionine via ornithine decarboxylase 1 (ODC1) and S-adenosylmethionine decarboxylase (AMD) [2]. Polyamines exist in all kingdom of life and single nucleotide polymorphisms resulting in alterations of polyamine metabolism have been implicated in a number of human diseases including mental illness and cancer [3, 4]. Polyamines appear to regulate gene expression, cell proliferation and stress responses due to their ability to bind to nucleic acids (both DNA, RNA), alter posttranslational modification and regulate ion channels [3, 4]. Numerous studies have alluded to a role of polyamines in regulating gene expression due to their polycationic nature and ability to function as a sink to S-Adenosylmethionine and Acetyl-coA, both critical meatabolites for histone modifications [5-8]. Intracellular polyamines and their analogues are also known to inhibit lysine-specific demethyltransferases such as LSD1 [9]. Despite its relevance in human disease and extensive clinical interest [10, 11], the role of the intrinsic polyamine pathway in immune cells is largely unexplored. Recent work demonstrated that the polyamine pathway and its connection to hypusination can regulate OXPHOS in macrophage [12], suggesting the relevance of this pathway in immune cells. The current study showed that enzymes of the polyamine pathway are suppressed and cellular polyamine content is significantly lower in regulatory T cells and non-pathogenic Th17 cells (Th17n) as compared to Th17 cells in the proinflammatory state (Th17p) due to alternative fluxing. Perturbation of the polyamine pathway in Th17 cells suppressed canonical Th17 cytokines and promoted Foxp3 expression, shifting the Th17 cell transcriptome in favor of Tregs-like state. Applicants demonstrated that the polyamine pathway is critical in maintaining the Th17-specific chromatin landscape against the induction of Tregs-like program. Consistent with the cellular phenotype, chemical inhibition and genetic perturbation of the polyamine pathway in T cells restricted the development of autoimmune responses in the EAE model.

### Identifying the Polyamine Pathway as a Candidate in Regulating Th17 Cell Functional State

To better analyze the metabolic landscape of Th17 cells in association with their functional state, Applicants first used two approaches: untargeted metabolomics (FIG. 17) and standard analysis of single-cell RNAseq data (FIG. 15G,H). For both analyses, Applicants compared Th17 cells differentiated from naïve CD4+ T cells using two combinations of cytokines: IL-1b+IL-6+IL-23 (Th17p) and TGFb+IL-6 (Th17n) that Applicants previous reported to either promote or restrict Th17 cell pathogenicity respectively in the context of the EAE model, and therefore represents the two extremes of functional state of Th17 cells [1, 13]. Untargeted metabolomics identified 1375 (out of 7436) metabolic features to be differentially expressed between Th17n and Th17p (FIG. 17). Most of the differentially expressed features are of lipid nature, consistent with the previous finding that lipid biosynthesis is a key regulator of Th17 cell functions [1]. Next, Applicants evaluated the metabolic transcriptome of sorted IL-17-GFP+Th17 cells differentiated in vitro as was previously published [14]. Similar to other cellular systems, metabolic genes show correlation with genes that are associated with Th17 cell function (FIG. 1511). Despite the clear metabolic differences revealed using metabolomics or transcriptomics, pin-pointing critical pathways is challenging.

To circumvent challenges in identifying metabolic pathways using traditional approach, Applicants next investigated the metabolic circuitry of Th17 cells using COMPASS (FIG. 15I,C), analyzing the same single cell RNAseq dataset from sorted IL-17-GFP+Th17 cells [14]. COMPASS analysis showed that among those metabolic reactions significantly correlated with Th17 cell pathogenicity, the polyamine pathway stands out as a critical pathway (FIG. 15I). Based on COMPASS prediction, Applicants constructed a data-driven metabolic network surrounding the polyamine pathway and found that there is a significant tendency of metabolic flux away from polyamine biosynthesis in Th17 cells associated with the regulatory functional state. (FIG. 15C). Applicants conclude that flux into polyamine biosynthesis may be associated with the inflammatory functional state of Th17 cells.

### Cellular Polyamines are Suppressed in Regulatory T Cells and Th17 Cells at the Regulatory State

To investigate the polyamine metabolic process (FIG. 15D), Applicants first asked whether critical enzymes of this pathway is differentially expressed in different CD4+ T cell subsets. Ornithine Decarboxylase 1 (ODC1) and Spermidine/Spermine N1 Acetyltransferase 1 (SAT1) are the rate-limiting enzymes of polyamine biosynthesis and catabolic processes respectively. ODC1 catalyzes ornithine to putrescine, the first step of the polyamines biosynthesis; whereas SAT1 regulates the intracellular content of polyamines and their transport out of the cell. Applicants observed that SAT1, but not ODC1 is suppressed in Th17n as compared to Th17p cells. The enzymatic activity of ODC1 can be regulated by ornithine decarboxylase antizyme 1 (OAZ1), Applicants did not find OAZ1 level to be significant different between Th17n and Th17p (data not shown). Intriguingly, both ODC1 and SAT1 expression are suppressed in inducible Tregs, whereas Ass1, an enzyme upstream of the polyamine biosynthesis pathway is upregulated, consistent with COMPASS-predicted alternative flux in the polyamine neighborhood (FIG. 15J). Collectively, these data suggest the polyamine pathway may be associated with regulatory functional state beyond Th17 cells.

As the polyamine pathway is regulated beyond the transcriptional level similar to most metabolic pathways, next, Applicants directly measured total cellular polyamine content using an enzymatic assay (Material and Methods). Compared to Th17p cells, Applicants found that Tregs and Th17n have significantly reduced levels of total polyamines (FIG. 15K), reflective of either reduced import, biosynthesis or increased export of polyamines in these cells.

To further investigate the concentrations and activities of different polyamines in Th17 cells at different functional state, Applicants applied both targeted metabolomics and carbon tracing approach. Th17n and Th17p cells are differentiated as previously described for 68 hours and the amount of polyamines and related precursors in cell and media are measured by LC/MS (FIG. 15E and FIG. 17B). Applicants observed that while the total amount of cellular ornithine, precursor to polyamines, are comparable between Th17n and Th17p, there is a significant increase of putrescine and acetyl-putrescine content in Th17p cells (FIG. 15E), indicative of increased activity of this pathway in the proinflammatory state of Th17 cells consistent with the enzymatic assay. Of note, cellular spermidine (or acetyl-spermidine) content is not different whereas spermine was not detected (FIG. 15E). The reduced putrescine and its acetyl form in Th17n cells are not due to increased export, as Applicants observed very little polyamines in the media in either Th17n or Th17p cells (FIG. 17B). These data suggest that the altered SAT1 expression at the transcriptional level is likely a consequence and not cause of the changes in the polyamine pathway in Th17 cells at different functional state.

To directly investigate polyamine biosynthesis, Applicants cultured differentiated Th17n and Th17p cells in the presence of low amount of C13 labeled arginine, which can be used to synthesize ornithine, a precursor to the polyamine pathway (FIG. 15D). Cells were harvested for LC/MS at 24 hours post addition of arginine, a time frame optimized for detection of accumulation of cellular polyamine. Applicants observed higher accumulation of putrescine, acetyl-putrescine and acetyl-spermidine in Th17p cells (FIG. 15F), consistent with a more active polyamine biosynthesis pathway in the proinflammatory state of Th17 cells. Importantly, Applicants observed that instead of channeling into polyamine biosynthesis, C13 labeled arginine is channeled into guanidinoacetic acid and creatine in Th17n cells, consistent with COMPASS prediction (FIG. 17C). Applicants conclude that alternative flux hinged on polyamine biosynthesis is associated with the functional state of Th17 cells.

### Inhibiting ODC1 or SAT1 Restricts Th17 Cell Function in a Putrescine Dependent Manner

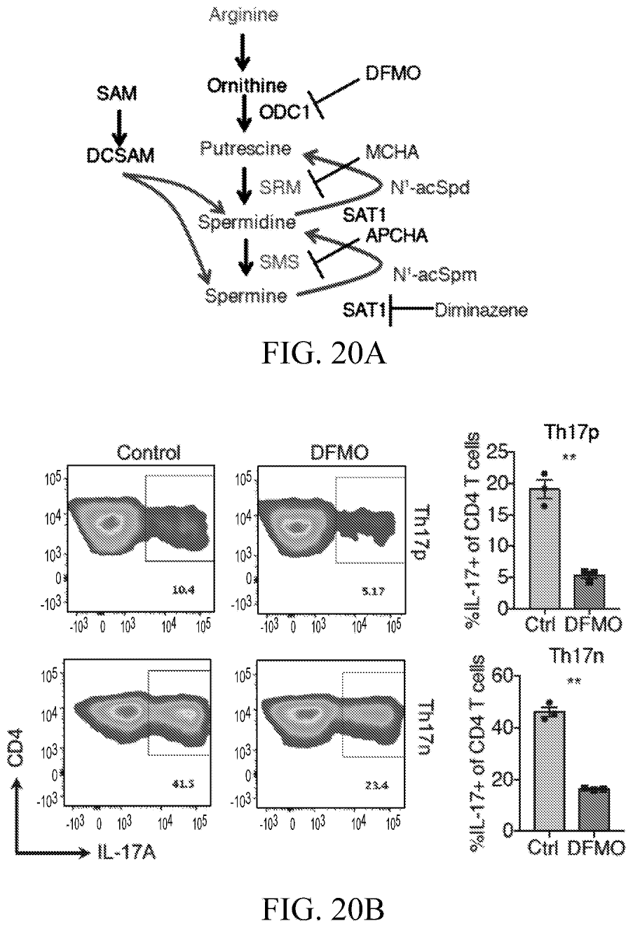

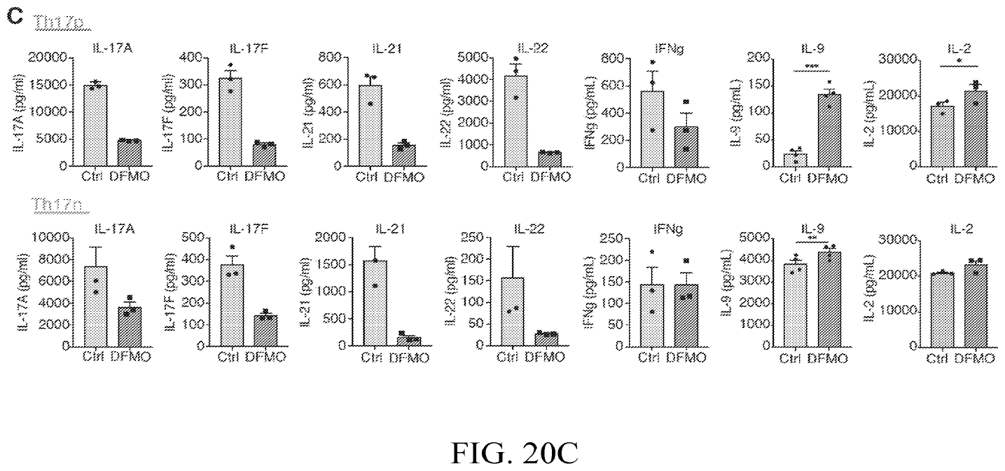

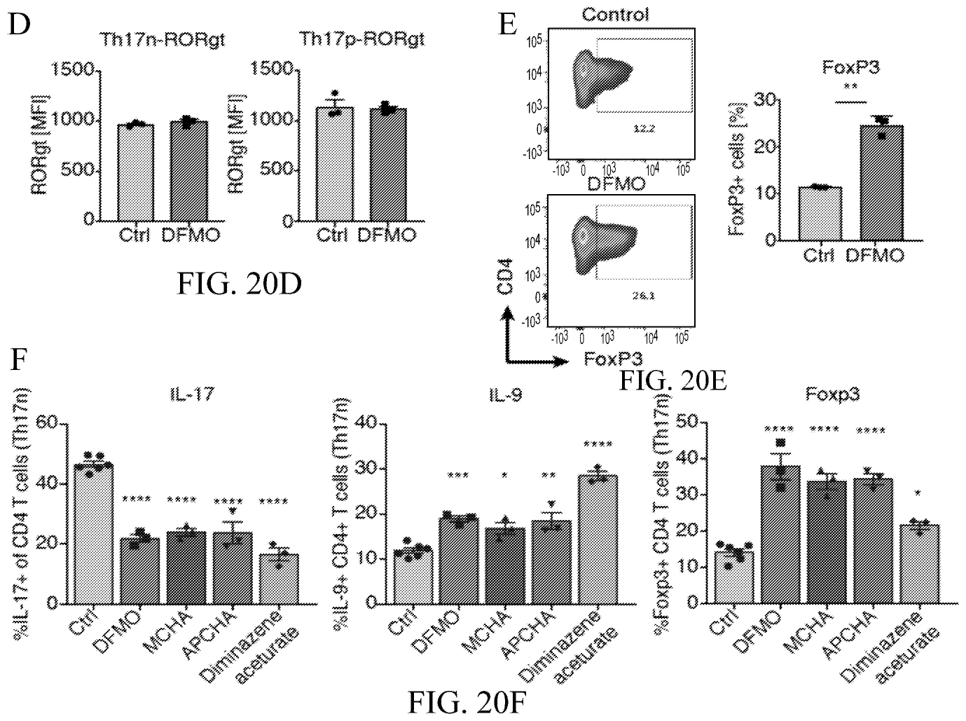

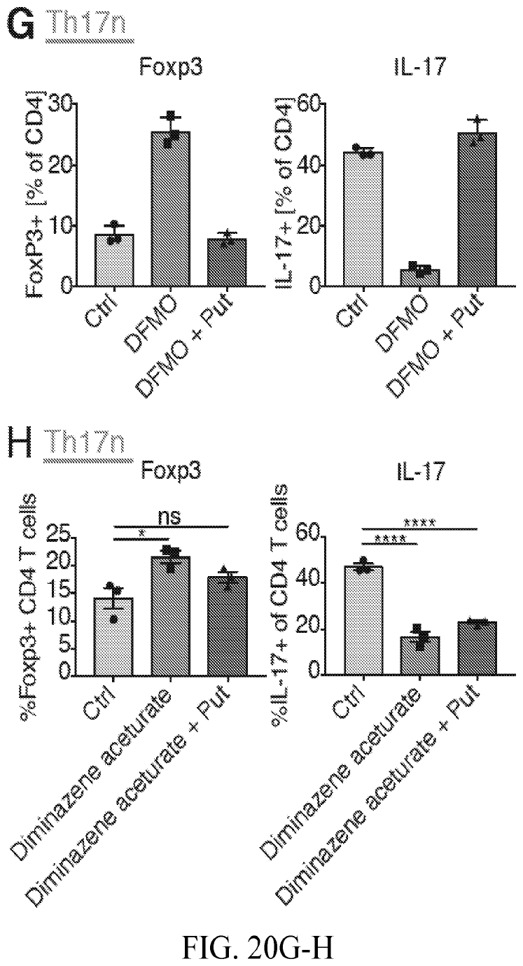

To investigate the functional relevance, Applicants used inhibitors of the polyamine pathway and studied their effects on Th17 cells at different functional state differentiated in vitro. Applicants first used difluoromethylornithine (DFMO), a competitive inhibitor of ODC1 (FIG. 20A). Applicants confirmed the effect of DFMO on in vitro differentiated Th17n and Th17p cells by using enzymatic assays which showed suppression of polyamines in both cell types (FIG. 18A). At an optimized concentration where Applicants observed similar viability between control and treatment, Applicants observed that DFMO significantly inhibited IL-17 expression in both Th17n and Th17p cells by intracellular staining and flow cytometry analysis (FIG. 20B). Consistently, DFMO inhibited canonical Th17 cytokines such as IL-17A, IL-17F, IL-21 and IL-22, while promoted IL-9 expression in supernatant from both Th17n and Th17p cultures (FIG. 20C). DFMO did not consistently influence, IFNg, TNFa, IL-13, IL-10 or IL-5 expression (FIG. 20C and FIG. 18B). The inhibition of IL-17 does not appear to be solely related to regulation of IL-2 production [15] as DFMO did not influence IL-2 expression in Th17p cells (FIG. 20c). Polyamines can influence cell proliferation. While Applicants did observe less cell proliferation in cultures treated with DFMO in some experiments, the frequency of IL-17+ cells are significantly reduced in cells that have divided just once (data not shown), suggesting DFMO can regulate Th17 cell function independent of cell proliferation.

To determine whether DFMO inhibited Th17 cell differentiation, Applicants measured the expression and activity of transcription factors. Interestingly, DFMO did not consistently alter Rorgt expression (FIG. 20D). The inhibition of IL-17 is also not due to reduced activity of Stat3 or increased Foxo1 activity, both are critical regulators of Th17 cell function, as DFMO inconsistently regulated pStat3 and promoted pFoxo1(5256) in both types of Th17 cells, which would have resulted in net increase in IL-17 expression (FIG. 18C). As polyamine concentrations are reduced in regulatory T cells, Applicants asked whether DFMO can regulate Foxp3 expression in Th17 cells. Applicants observed increased frequency of Foxp3+ cells in Th17n but not Th17p conditions (FIG. 20E and data not shown).

To determine whether other enzymes of the polyamine pathway could play a similar role in regulating Th17 cell function, Applicants used inhibitors of spermidine synthase (SRM), spermine synthase (SMS), and SAT1 (FIG. 20A). Similar to DFMO, inhibitors of any of the polyamine biosynthesis enzymes resulted in suppression of IL-17 and upregulation of IL-9 and Foxp3 expression (FIG. 20F). Surprisingly, inhibiting SAT1, rate-limiting enzyme of polyamine acetylation and export, had reduced but similar effects as compared to DFMO (FIG. 20F). SAT1 perturbation was previously reported to have a feedback on ODC1 activity and vice versa [6, 7, 16]. Consistent with this finding, Applicants found that DFMO inhibition consistently suppressed SAT1 expression in both Th17n and Th17p cells (FIG. 18D). Thus, it may be the flux of polyamines and not the metabolites themselves per se that modulate Th17 cell function.

Applicants next confirmed that the effect of DFMO is through the inhibition of ODC1 as addition of putrescine to cells treated with DFMO completely reversed their phenotype (FIG. 20G). Interestingly, addition of putrescine also partially reversed the upregulation of Foxp3, but not suppression of IL-17, by SAT1 inhibitor (FIG. 20H), suggesting putrescine flux may be particularly important in the control of the regulatory program in Th17 cells. These observations are consistent with a role of the polyamine pathway in regulating Th17 functional state but not necessarily differentiation, but genome-wide profiling would be necessarily to further support this claim.

DFMO restricts Th17-cell transcriptome and epigenome in favor of Treg-like state

To gain mechanistic insight on the effects of inhibiting polyamine biosynthesis in Th17 cells, Applicants performed RNAseq on Th17n, Th17p and compared that to iTregs treated with ctrl or DFMO. DFMO has profound impact on the transcriptome of all Th cell lineages, clearly driving cells towards Treg cells in principal component analysis (FIG. 21A). To gain further insights, Applicants determined the effect of DFMO comparing confined transcriptome space: 1) defined by Th17n and Th17p cells such that it characterizes distinct functional state (FIG. 19B); and 2) defined by Th17 cells and iTregs (FIG. 21B). In the context of Th17 cell functional state (FIG. 19B), Applicants divided detectible transcriptome space into those upregulated in Th17n cells (regulatory state), upregulated in Th17p cells (proinflammatory state) and those not significantly different. Upon DFMO treatment, Applicants observed a significant upregulation of the regulatory state and downregulation of the proinflammatory state in Th17p cells (FIG. 19B), consistent with the polyamine pathway being a positive regulator inflammation driven by Th17 cells. It should be noted that further inhibiting polyamine biosynthesis in Th17n cells where this pathway is already less active actually promoted the proinflammatory module suggesting a nuisance effect.

As Applicants observed Foxp3 upregulation at least in Th17n cells (FIG. 20E) and a shift towards Treg cells in PCA (FIG. 21A) with DFMO treatment, Applicants determined the effect of DFMO In the transcriptome space defined by iTregs and Th17 cells. Applicants observed that DFMO suppressed Th17 cell specific transcriptome but promoted Treg-specific transcriptome in both Th17n and Th17p cells as compared to those genes not significantly altered (FIG. 21B, C). Specifically, canonical Th17 cell genes such as Il17a, Il17f and Il23r are significantly suppressed whereas Treg related genes such as Foxp3 is upregulated (FIG. 21C). These results are consistent with the polyamine pathway being important in restricting iTreg-like transcriptome in Th17 cells at both functional states.

The profound impact of DFMO on transcriptome prompted Applicants to investigate the mechanism by which the polyamine pathway regulates Th17 cell functions. As DFMO does not appear to consistently restrict phosphorylation of key Th17 cell regulators (FIG. 18C), Applicants turned to known functions of polyamines in regulating epigenome. Consistent with a role of the polyamine pathway in chromatin modification, Applicants observed significant changes in expression of many chromatin modifiers (FIG. 21D).

To directly test the hypothesis that the polyamine pathway may regulate Th17 function by altering histone modification and DNA accessibility, Applicants measured chromatin accessibility by performing ATACseq in Th17p, Th17n and iTregs cells treated with either control or DFMO (Material and Methods). Overall, Applicants observed significant changes in accessible peaks in all Th cells analyzed in response to DFMO treatment (FIG. 19C). Next, Applicants asked whether DFMO preferentially altered accessibility to regions specific to Th17 cells and iTregs. To this end, Applicants divided all accessible peaks into three spaces as Applicants did to the RNAseq data (FIG. 21E): those more accessible in Th17 cells, more accessible in iTregs, and those not differentially accessible. Consistent with the gene expression data, Applicants observed significant shift towards more restricted landscape in Th17 specific regions in favor of more accessible landscape in Treg specific regions (FIG. 21E). This is not necessarily expected as the suppression of the Th17 canonical program at the transcriptome level could be due to activities of a transcription factor network. This suggests that the polyamine pathway indeed can shape epigenome landscape in a manner consistent with transcriptome changes.

Next, Applicants asked whether the chromatin accessibility changes could be driving the transcriptome regulation. To this end, Applicants first examined Th17-specific and iTreg-specific genomic regions corresponding to Il17a-Il17f, Il23r and Foxp3 (FIG. 21F, G), all of which are suppressed or upregulated respectively by inhibiting the polyamine pathway. Applicants aligned the IGV plots with the ATAC-seq peaks from ChIPseq data for Rorgt (material and methods and [17]), the “master” regulator of Th17 cells. Applicants observed that DFMO significantly restricted peaks in the promoter and intergenic regions of Il17a-Il17f that corresponds to Rorgt binding site known to regulate IL17 expression (FIG. 21F, upper panel). Interestingly, the differentially accessible peaks driven by DFMO differs in Th17n and Th17p cells despite them all being Rorgt binding site, suggesting alternative co-factors must be utilized. On the other hand, there is no significant difference in ATACseq peaks in the Il23r region (FIG. 21F, lower panel), suggesting not all of the canonical Th17 cell genes are suppressed by restricting chromatin accessibility. Similarly, there are no differentially expressed peaks near regulatory elements for Foxp3 such as CNS2 [18] in response to DFMO (FIG. 21G), suggesting alternative mechanism. It should be noted that in Th17n cells Applicants did observe differential accessibility near the C-terminus of Foxp3 gene (FIG. 21G), which could be a suppressive element as it is preferentially closed in iTreg cells as compared to Th17 cells or in DFMO treated Th17n cells. To expand this investigation globally, Applicants plotted the effect of DFMO on gene expression (log fold change in RNAseq) against chromatin accessibility (log fold change in genes near ATACseq peaks) (FIG. 19C, D). Applicants observed low correlation coefficiency in total ATACseq peaks and such coefficiency did not improve when Applicants restrict the analysis to ATACseq peaks differentially regulated by DFMO. Thus, Applicants conclude that while DFMO can indeed shape chromatin accessibility in favor of iTreg epigenome landscape, it is not the only factor that drives its regulation on gene expression.

Upregulation of Foxp3 in DFMO Treatment is cMAF Dependent

To investigate what transcription factor network may be responsible for the suppression of Th17 specific program and upregulation of iTreg program, Applicants performed motif analysis in the ATACseq peaks using existing ChIPseq data (FIG. 21H). First, Applicants analyzed those Th17-specific regions in both Th17p and Th17n cells (FIG. 21H and FIG. 19E, left panels). As expected, accessible regions in control treated Th17p cells are enriched for motifs for known regulator such as RORgt, RORa, STAT3 and IRF4. On the other hand, inhibiting the polyamine pathway in Th17p cells didn't influence enrichment of RORgt motifs, suggesting the polyamine pathway functions in an RORgt-independent fashion. Instead, Applicants observed loss of enrichment for RORa, STAT3 and IRF4, in favor of transcription factors involved in T cell activation and apoptosis such as the wnt signaling pathway (TCF3 and VentX [19, 20]) and MEF2 [21], as well as AP-1 complexes (Jun/Fos, BATF/JunB) required for Th17 differentiation [22, 23] (FIG. 21H, left panel). The increased potential for MEF2 transcription is consistent with the suppression of HDAC4 (FIG. 21D), a known repressor of MEF2 activities [24]. Interestingly, in Th17n cells, DFMO treatment resulted in enrichment of motif for a different set of transcription factors, including IRF4 and STAT3, seemingly opposite to the DFMO effect in Th17p cells (FIG. 19E, left panel). This is consistent with the nuisance effect of DFMO on Th17n transcriptome in the context of Th17 cell functional state (FIG. 19B). It should be noted that motifs enriched in the accessible regions in control-treated Th17n cells are completely different as compared to Th17p cells, highlighting different set of transcriptional network must be governing the Th17 program in these two functional state of Th17 cells. Thus, Applicants conclude that the polyamine pathway contribute to gene regulation based on existing transcriptional framework at least in the context of the core Th17 program.

Next, Applicants investigated the accessible regions that are iTreg-specific (FIG. 21H and FIG. 19E, right panels). As expected, little motif enrichment was found in control treated Th17p or Th17n cells. With DFMO treatment, however, Applicants observed significant enrichment of BATF and cMAF motifs along with a number of POU domain containing transcription factors in both Th17n and Th17p cells. The enrichment of BATF motifs also in the Th17-specific regions made accessible by DFMO suggest the function of the polyamine pathway may be associated with BATF activity. However, BATF is a pioneering factor for Th17 differentiation [22, 25] and the loss of BATF would result in loss of Th17 cell program entirely, making rescue experiment difficult to interpret. On the other hand, cMAF is a known regulator of Treg function [26], Applicants therefore focused on whether cMAF is a relevant mediator downstream of the polyamine pathway. To determine whether cMAF plays a role, Applicants used conditional cMAF knockout mice. Applicants analyzed the effect of DFMO on Th17 cells differentiated from naïve CD4 T cells isolated from control or cMAF^{fl/fl}CD4^{cre} mice (FIG. 21I). Applicants observed that cMAF deletion partially rescued the effect of DFMO on Foxp3 upregulation and, as expected, did not impact the expression of IL-17.

### ODC1 and SAT1 Perturbation Alleviated EAE

To investigate the relevance of the polyamine pathway in vivo (FIG. 16A), Applicants took two approaches: chemical inhibition of ODC1 and T-cell specific genetic deletion of SAT1 in the context of EAE development (FIG. 16). As targeting either ODC1 or SAT1 (FIG. 20) resulted in reduced canonical Th17 cytokines and upregulation of Foxp3 in vitro, Applicants expect both approaches to alleviate EAE.

Applicants first analyzed the role of ODC1 inhibition by adding DFMO in drinking water for mice immunized with MOG/CFA for the induction of EAE (Material and Methods). DFMO significantly delayed EAE onset and severity (FIG. 16H). Consistently, Applicants observed significantly reduced antigen-specific response in the draining lymph node of DFMO treated animals (FIG. 16I). Further analysis of lymphocytes isolated from CNS showed no difference in the frequency of cytokine producing cells but increased Foxp3+CD4+ T cells (FIG. 16J and data not shown), consistent with the polyamine biosynthesis pathway being an important positive regulator of autoimmune inflammation.

Next, Applicants studied the T cell intrinsic effect of the polyamine pathway in EAE development. Applicants generated SAT1 conditional deletion mice in T cells (SAT 1^{fl/fl}CD4^{cre}). Applicants confirmed that genetic deletion of SAT1 in T cells resulted in loss of polyamine acetylation as reflected in the reduced acetyl-putrescine and acetyl-spermidine (FIG. 16C). It is important to note that loss of SAT1 also resulted in reduced level of putrescine in Th17 cells, likely through a feedback mechanism. This is consistent with reports in other cell types [16] and the in vitro inhibitor data (FIG. 20), suggesting similar effect of DFMO and SAT1 deletion in the context of T cell biology. Indeed, Applicants observed significantly delayed onset and severity of EAE in SAT1^{fl/fl}CD4^{cre} mice (FIG. 16D). Similar to DFMO global treatment, Applicants observed restricted antigen-specific recall response as measured by T cell proliferation (FIG. 16E). In addition, while Applicants did not observe significant changes in antigen-specific cytokine production (FIG. 16G), there is a significant upregulation of Foxp3⁺CD4⁺ T cells in SAT1^{fl/fl}CD4^{cre} mice (FIG. 16F). Thus, using both chemical and genetic approaches at multiple levels, Applicants demonstrated that the polyamine pathway is an important mediator of autoimmune inflammation.

### Example 5 Materials and Methods

Mice. C57BL/6 wildtype (WT) were obtained from Jackson laboratory (Bar Harbor, Me.). CD4Cre SAT1flox mice were kindly provided by Dr. Soleimani ( ). For experiments, mice were matched for sex and age, and most mice were 6-10 weeks old. For EAE experiment, littermate control WT was used in comparison to CD4Cre SATlflox mice in one experiment which produced similar results compared to WT from Jackson. All experiments were conducted in accordance with animal protocols approved by the Harvard Medical Area Standing Committee on Animals or BWH IACUC.

Single-cell RNAseq data acquisition and analysis. Applicants prepared single-cell mRNA SMART-Seq libraries using microfluidic chips (Fluidigm C1) for single-cell capture, lysis, reverse transcription, and PCR amplification, followed by transposon-based library construction. For quality assurance, Applicants also profiled corresponding population controls (>50,000 cells for in vitro samples; 2,000-20,000 cells for in vivo samples, as available), with at least two replicates for each condition. RNA-seq reads were aligned to the NCBI Build 37 (UCSC mm9) of the mouse genome using TopHat (Trapnell et al., 2009). The resulting alignments were processed by Cufflinks to evaluate the abundance (using FPKM) of transcripts from RefSeq (Pruitt et al., 2007). Applicants used log transform and quantile normalization to further normalize the expression values (FPKM) within each batch of samples (i.e., all single-cells in a given run). To account for low (or zero) expression values Applicants added a value of 1 prior to log transform. Applicants filtered the set of analyzed cells by a set of quality metrics (such as sequencing depth), and added an additional normalization step specifically controlling for these quantitative confounding factors as well as batch effects. The analysis is based on ˜7,000 appreciably expressed genes (fragments per kilobase of exon per million (FPKM)>10 in at least 20% of cells in each sample) for in vitro experiments and ˜4,000 for in vivo ones. Applicants also developed a strategy to account for expressed transcripts that are not detected (false negatives) due to the limitations of single-cell RNA-seq (Deng et al., 2014; Shalek et al., 2014). The analysis (e.g., computing signature scores, and principle components) down-weighted the contribution of less reliably measured transcripts. The ranking of regulators shown in FIG. 15 is based on having a strong correlation to at least one of the founding signature genes, and in addition, the significance of the overall pattern relative to the proinflammatory vs. regulatory signature by comparing the aggregates pattern across the individual correlations to shuffled data.

T cell differentiation culture & Flow cytometry. Naïve CD4+CD44-CD62L+CD25-T cells were sorted using BD FACSAria sorter and activated with plate-bound anti-CD3 (1 μg/ml) and antiCD28 antibodies (2 μg/ml) in the presence of cytokines at a concentration of 5×10⁵ cells/ml. For T cell differentiations the following combinations of cytokines were used: pathogenic Th17: 25 ng/ml rmIL-6, 20 ng/ml rmIL-1b (both Miltenyi Biotec) and 20 ng/ml rmIL-23 (R&D systems); non-pathogenic Th17: 25 ng/ml rmIL-6 and 2 ng/ml of rhTGFb1 (Miltenyi Biotec); iTreg: 2 ng/ml of rhTGFb1; Th1: 20 ng/ml rmIL-12 (R&D systems); Th2: 20 ng/ml rmIL-4 (Miltenyi Biotec). For differentiation experiments, cells were harvested at 72 hours and were performed in the presence or absence of 200 mM DFMO or 2.5 mM Putrescine (both Sigma) as indicated.

Intracellular cytokine staining was performed after incubation for 4-6h with Cell Stimulation cocktail plus Golgi transport inhibitors (Thermo Fisher Scientific) using the BD Cytofix/Cytoperm buffer set (BD Biosciences) per manufacturer's instructions. Transcription factor staining was performed using the Foxp3/Transcription Factor Staining Buffer Set (eBioscience).

Proliferation was assessed by staining with CellTrace Violet (Thermo Fisher Scientific) per manufacturer's instructions. Apoptosis was assessed using Annexin V staining kit (BioLegend). Phosphorylation of proteins to determine cell signaling was performed with BD Phosflow buffer system (BD bioscience) as per manufacturer's instructions.

Legendplex. Cytokine concentrations in supernatants of in vitro cultures were analyzed by the LegendPlex Mouse Th Cytokine Panel (13-plex) (BioLegend) according to the manufacturer's instructions and analyzed on a FACS LSR II (BD Biosciences).

qPCR. RNA was isolated using RNeasy Plus Mini Kit (Qiagen) and reverse transcribed to cDNA with iScript cDNA Synthesis Kit (Bio-Rad). Gene expression was analyzed by quantitative real-time PCR on a ViiA7 System (Thermo Fisher Scientific) using TaqMan Fast

Advanced Master Mix (Thermo Fisher Scientific) with the following primer/probe sets: Il-17a (Mm00439618_m1), Il-17f (Mm00521423_m1), Foxp3 (Mm00475162_m1), Tead1 (Mm00493507_m1), Taz (Mm00504978_m1), Sat1 (Mm00485911_g1) and Actb (Applied Biosystems). Expression values were calculated relative to Actb detected in the same sample by duplex qPCR.

Antibodies. All other flow cytometry antibodies were purchased from Biolegend.

Experimental Autoimmune Encephalomyelitis (EAE). For active EAE immunization, MOG35-55 peptide was emulsified in complete freund adjuvant (CFA). Equivalent of 40 μg MOG peptide was injected per mouse subcutaneously followed by pertussis toxin injection intravenously on day 0 and day 2 of immunization. Mice were treated with 0.5% DFMO in drinking water for 10 days as indicated. DFMO was replenished every third day.

Suppression assay. Freshly isolated naïve CD4⁺ T cells (4×10⁴) were stained with CellTrace Violet as described above and cultured with various differentiated T cells in a 1:1 or 2:1 ratio in 96-well round-bottom plates (Corning Inc.) in the presence of anti-CD3/CD28 beads (50×10³ beads/well; Invitrogen). After 72 hrs cells were analyzed to assess proliferation.

RNA-seq. For population (bulk) RNA-seq, in vitro differentiated T-cells were sorted for live cells and lysed with RLT Plus buffer and RNA was extracted using the RNeasy Plus Mini Kit (Qiagen). Full-length RNA-seq libraries were prepared as previously described [27] and paired-end sequenced (75 bp×2) with a 150 cycle Nextseq 500 high output V2 kit.

ATAC-seq. For population ATAC-seq, in vitro differentiated T-cells were sorted for live cells and froze down in Bambanker freezing media (Thermo Fisher Scientific).

Alignment of ATAC-Seq and Peak Calling. All ATAC-Seq reads were trimmed using Trimmomatic [28] to remove primer and low-quality bases. Reads <36 bp were dropped. Reads were then passed to FastQC [www.bioinformatics.babraham.ac.uk/projects/fastqc/] to check the quality of the trimmed reads. The paired-end reads were then aligned to the mm10 reference genome using bowtie2 [29], allowing maximum insert sizes of 2000 bp, with the “—no-mixed” and “—no-discordant” parameters added. Reads with a mapping quality (MAPA) below 30 were removed. Duplicates were removed with PicardTools, and the reads mapping to the blacklist regions and mitochondrial DNA were also removed. Reads mapping to the positive strand were moved +4 bp, and reads mapping to the negative strand were moved −5 bp following the procedure outlined in [30] to account for the binding of the Tn5 transposase.

Peaks were called using macs2 on the aligned fragments [31] with a qvalue cutoff of 0.001 and overlapping peaks among replicates were merged.

Tests of Differential Accessibility in ChARs. Differential accessibility was assessed using DESeq2 [32] on with a matrix of peaks by samples replacing the genes by samples matrix. Counts of Tn5 cuts were used instead of gene expression values. Peaks were considered differentially accessible if they had an adjusted pvalue <0.05.

Alignment of ChIP-Seq and Peak Calling. ChIP-Seq Peaks from Xiao et al 2014 [17] were transferred from mm9 to mm10 using the UCSC liftOver tool. Xiao et al 2014-RORyt www.ncbi.nlm.nih.gov/geo/query/acc.cgi?acc=GSM1350476; Xiao et al 2014—Foxp3 www.ncbi.nlm.nih.gov/geo/query/acc.cgi?acc=GSM1350486

ChIP-Seq replicates from Ciofani et al 2012 were downloaded and were trimmed using Trimmomatic [28] to remove primer and low-quality bases. Reads were then passed to FastQC [www.bioinformatics.babraham.ac.uk/projects/fastqc/] to check the quality of the trimmed reads. These single-end reads were then aligned to the mm10 reference genome using bowtie2 [29], allowing maximum insert sizes of 2000 bp, with the “—no-mixed” and “—no-discordant” parameters added. Reads with a mapping quality (MAPA) below 30 were removed. Duplicates were removed with PicardTools, and the reads mapping to the blacklist regions and mitochondrial DNA were also removed.

ChIP-Seq peaks were called in each replicate, versus a control sample, using macs2 [31] with a qvalue cutoff of 0.05.

Statistical Analysis. Unless otherwise specified, all statistical analyses were performed using the two-tail student t test using GraphPad Prism software. P value less than 0.05 is considered significant (P<0.05=*; P<0.01=**; P<0.001=***) unless otherwise indicated.

### Example 5 References

1. Wang, C., et al., CDSL/AIM Regulates Lipid Biosynthesis and Restrains Th17 Cell Pathogenicity. Cell, 2015. 163 (6): p. 1413-27.
2. Miller-Fleming, L., et al., Remaining Mysteries of Molecular Biology: The Role of Polyamines in the Cell. J Mol Biol, 2015. 427 (21): p. 3389-406.
3. Pegg, A. E., Mammalian polyamine metabolism and function. IUBMB Life, 2009. 61 (9): p. 880-94.
4. Pegg, A. E., Functions of Polyamines in Mammals. J Biol Chem, 2016. 291 (29): p. 14904-12.
5. Kraus, D., et al., Nicotinamide N-methyltransferase knockdown protects against diet-induced obesity. Nature, 2014. 508 (7495): p. 258-62.
6. Jell, J., et al., Genetically altered expression of spermidine/spermine N1-acetyltransferase affects fat metabolism in mice via acetyl-CoA. J Biol Chem, 2007. 282 (11): p. 8404-13.
7. Pegg, A. E., Spermidine/spermine-N(1)-acetyltransferase: a key metabolic regulator. Am J Physiol Endocrinol Metab, 2008. 294 (6): p. E995-1010.
8. Childs, A. C., D. J. Mehta, and E. W. Gerner, Polyamine-dependent gene expression. Cell Mol Life Sci, 2003. 60 (7): p. 1394-406.
9. Tamari, K., et al., Polyamine flux suppresses histone lysine demethylases and enhances IDI expression in cancer stem cells. Cell Death Discov, 2018. 4: p. 104.
10. Gerner, E. W. and F. L. Meyskens, Jr., Polyamines and cancer: old molecules, new understanding. Nat Rev Cancer, 2004. 4 (10): p. 781-92.
11. Sholler, G. L. S., et al., Maintenance DFMO Increases Survival in High Risk Neuroblastoma. Sci Rep, 2018. 8 (1): p. 14445.
12. Puleston, D. J., et al., Polyamines and eIF5A Hypusination Modulate Mitochondrial Respiration and Macrophage Activation. Cell Metab, 2019.
13. Lee, Y., et al., Induction and molecular signature of pathogenic TH17 cells. Nat Immunol, 2012. 13 (10): p. 991-9.
14. Gaublomme, J. T., et al., Single-Cell Genomics Unveils Critical Regulators of Th17 Cell Pathogenicity. Cell, 2015. 163 (6): p. 1400-12.
15. Bowlin, T. L., B. J. McKown, and P. S. Sunkara, The effect of alpha-difluoromethylornithine, an inhibitor of polyamine biosynthesis, on mitogen-induced interleukin 2 production. Immunopharmacology, 1987. 13 (2): p. 143-7.
16. Mounce, B. C., et al., Interferon-Induced Spermidine-Spermine Acetyltransferase and Polyamine Depletion Restrict Zika and Chikungunya Viruses. Cell Host Microbe, 2016. 20 (2): p. 167-77.
17. Xiao, S., et al., Small-molecule RORgammat antagonists inhibit T helper 17 cell transcriptional network by divergent mechanisms. Immunity, 2014. 40 (4): p. 477-89.
18. Li, X., et al., Function of a Foxp3 cis-element in protecting regulatory T cell identity. Cell, 2014. 158 (4): p. 734-748.
19. Gao, H., et al., VentX, a novel lymphoid-enhancing factor/T-cell factor-associated transcription repressor, is a putative tumor suppressor. Cancer Res, 2010. 70 (1): p. 202-11.
20. Le, Y., et al., The homeobox protein VentX reverts immune suppression in the tumor microenvironment. Nat Commun, 2018. 9 (1): p. 2175.
21. Youn, H. D., et al., Apoptosis of T cells mediated by Ca2+-induced release of the transcription factor MEF2. Science, 1999. 286 (5440): p. 790-3.
22. Schraml, B. U., et al., The AP-1 transcription factor Batf controls T(H)17 differentiation. Nature, 2009. 460 (7253): p. 405-9.
23. Yamazaki, S., et al., The AP-1 transcription factor JunB is required for Th17 cell differentiation. Sci Rep, 2017. 7 (1): p. 17402.
24. Wang, Z., G. Qin, and T. C. Zhao, HDAC4: mechanism of regulation and biological functions. Epigenomics, 2014. 6 (1): p. 139-50.
25. Karwacz, K., et al., Critical role of IRF1 and BATF in forming chromatin landscape during type 1 regulatory cell differentiation. Nat Immunol, 2017. 18 (4): p. 412-421.
26. Xu, M., et al., c-MAF-dependent regulatory T cells mediate immunological tolerance to a gut pathobiont. Nature, 2018. 554 (7692): p. 373-377.
27. Singer, M., et al., A Distinct Gene Module for Dysfunction Uncoupled from Activation in Tumor-Infiltrating T Cells. Cell, 2016. 166 (6): p. 1500-1511 e9.
28. Bolger, A. M., M. Lohse, and B. Usadel, Trimmomatic: a flexible trimmer for Illumina sequence data. Bioinformatics, 2014. 30 (15): p. 2114-20.
29. Langmead, B. and S. L. Salzberg, Fast gapped-read alignment with Bowtie 2. Nat Methods, 2012. 9 (4): p. 357-9.
30. Buenrostro, J. D., et al., Transposition of native chromatin for fast and sensitive epigenomic profiling of open chromatin, DNA-binding proteins and nucleosome position. Nat Methods, 2013. 10 (12): p. 1213-8.
31. Zhang, Y., et al., Model-based analysis of ChIP-Seq (MACS). Genome Biol, 2008. 9 (9): p. R137.
32. Love, M. I., W. Huber, and S. Anders, Moderated estimation of fold change and dispersion for RNA-seq data with DESeq2. Genome Biol, 2014. 15 (12): p. 550.

### Example 6—Glycolytic Pathways Regulate Th17 Pathogenicity

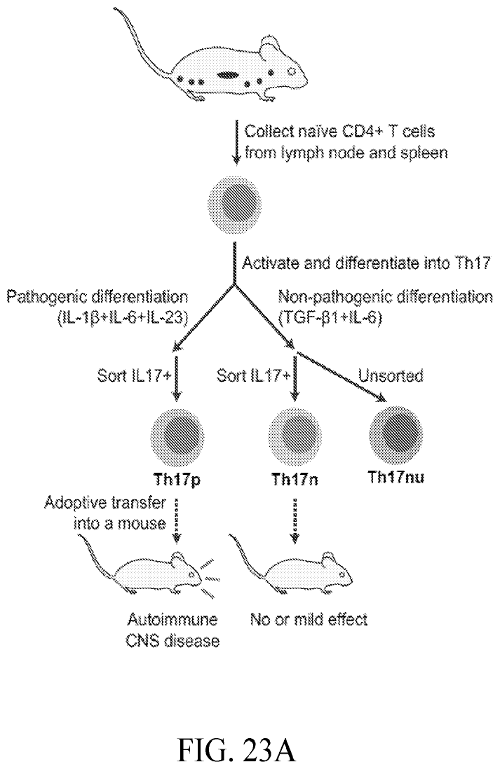

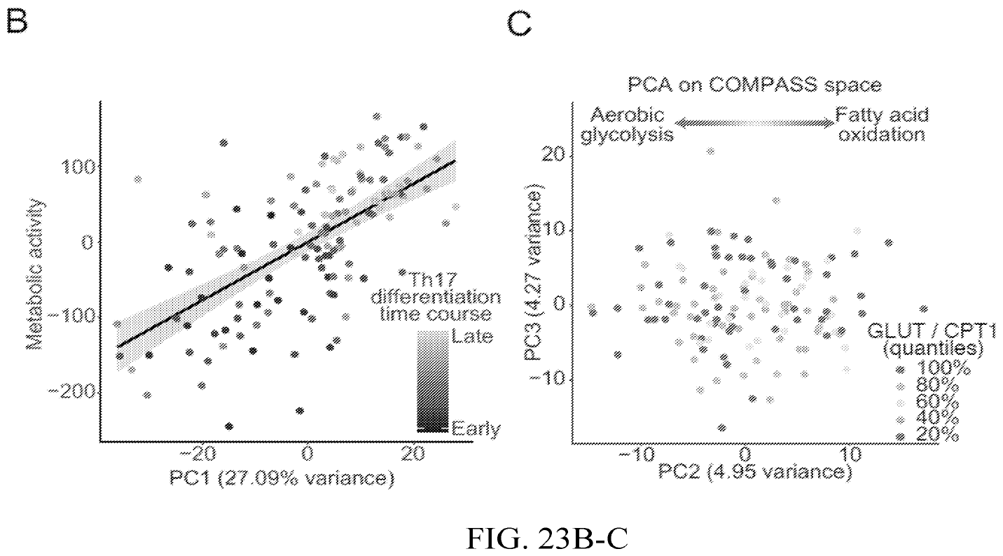

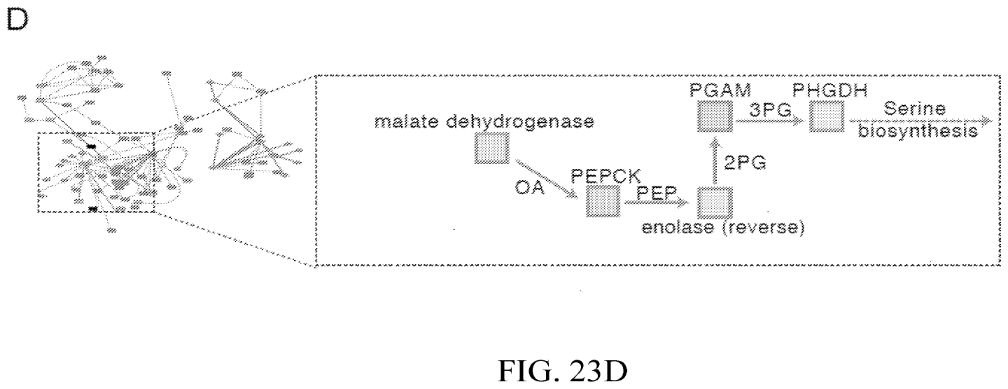

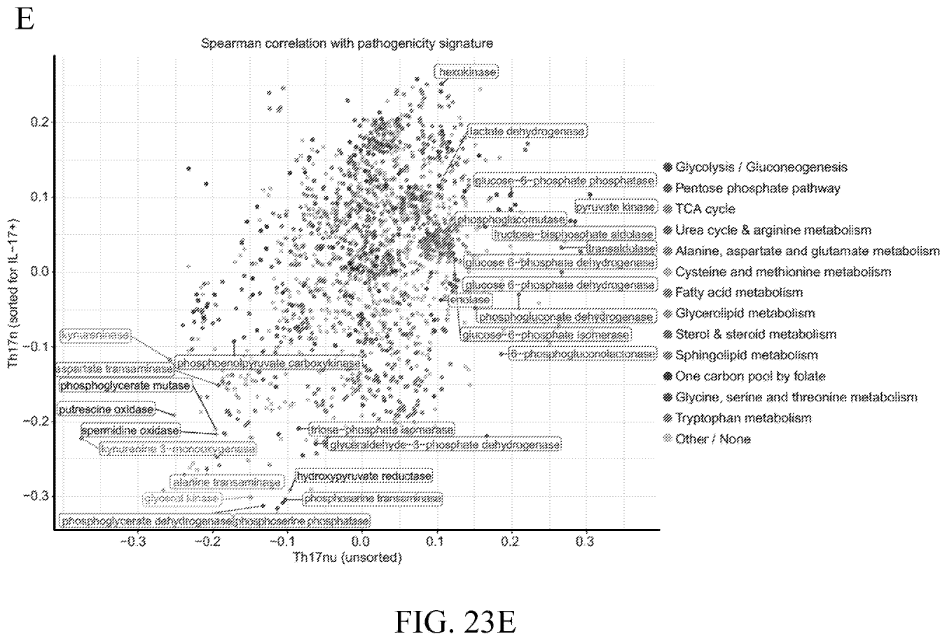

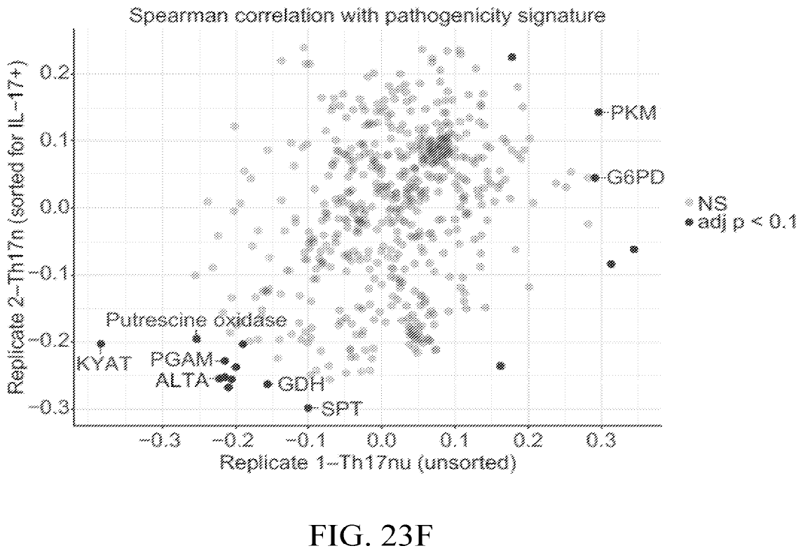

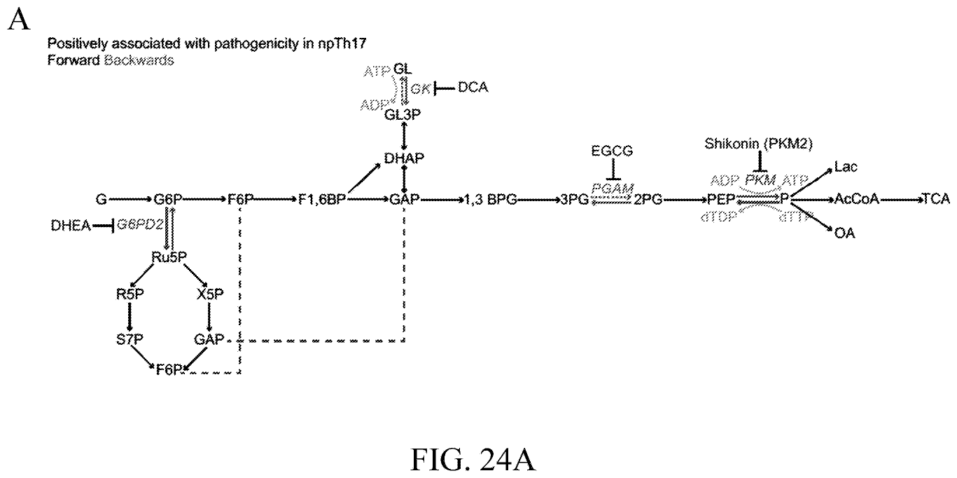

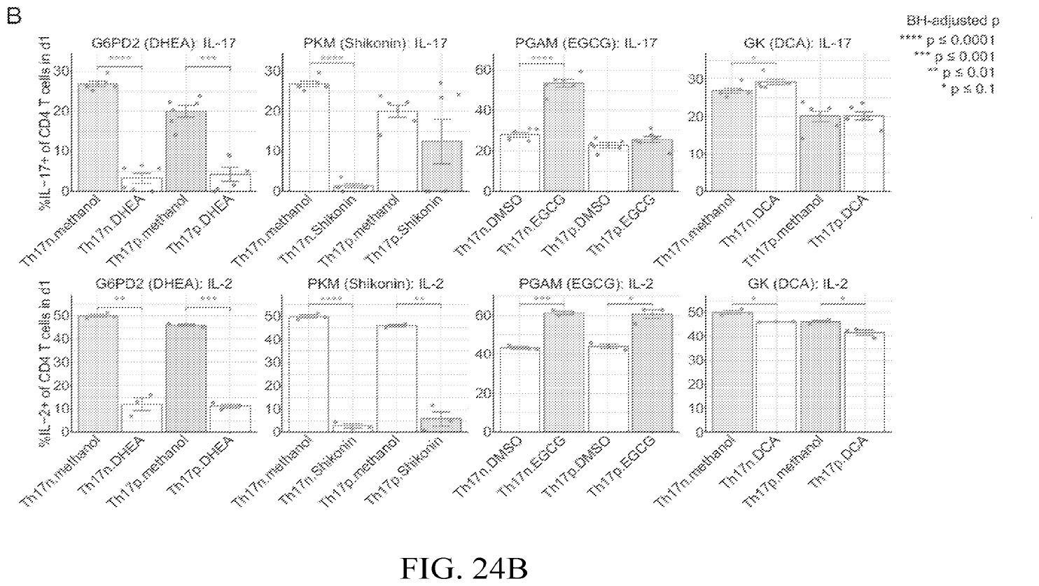

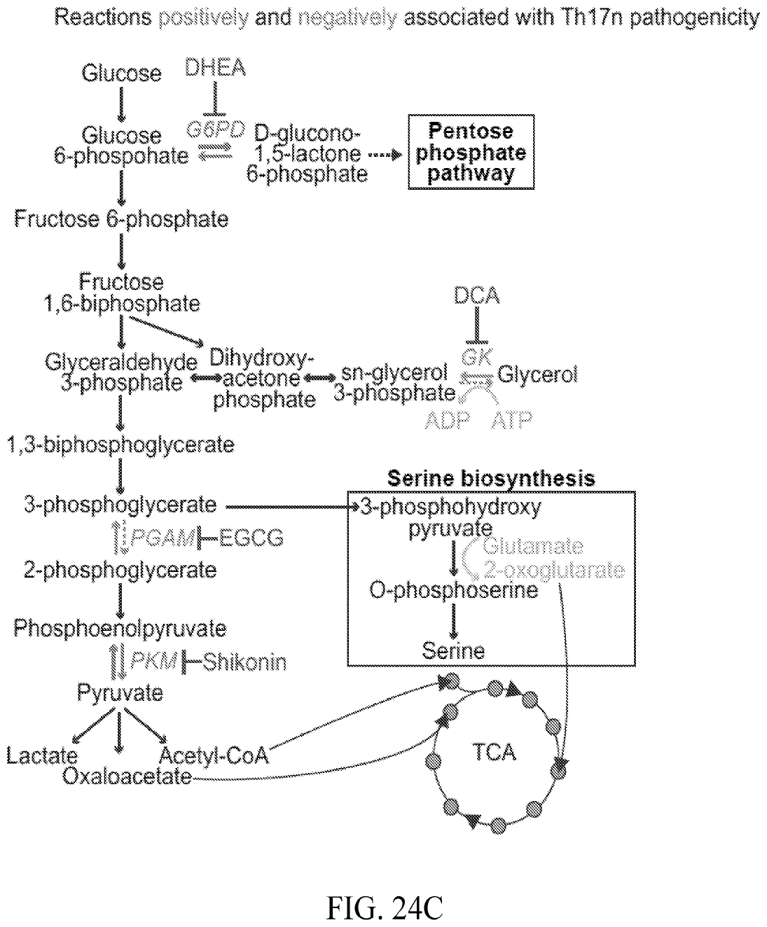

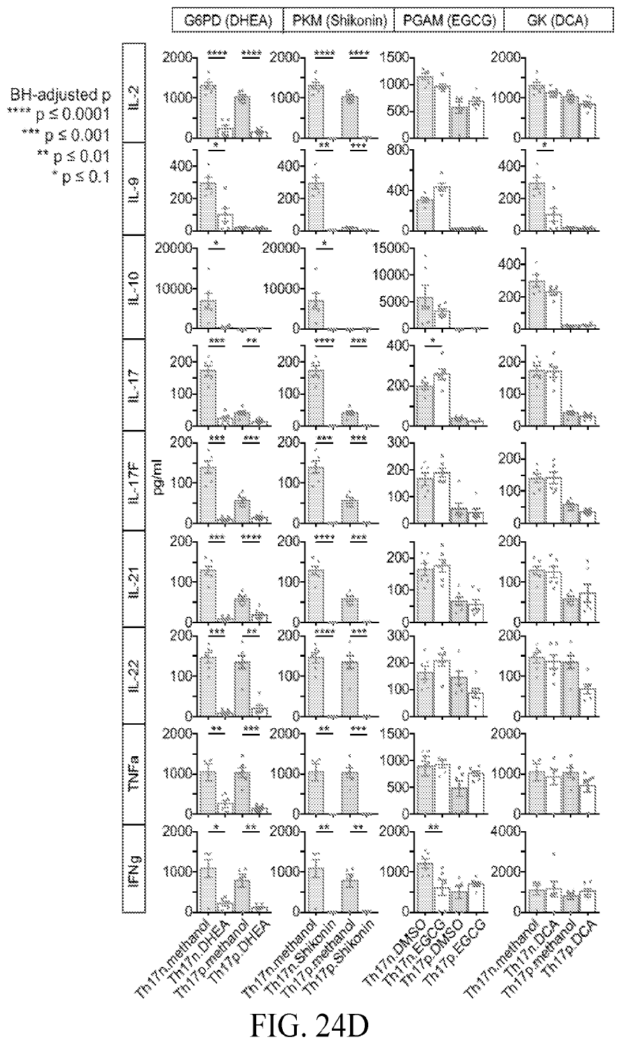

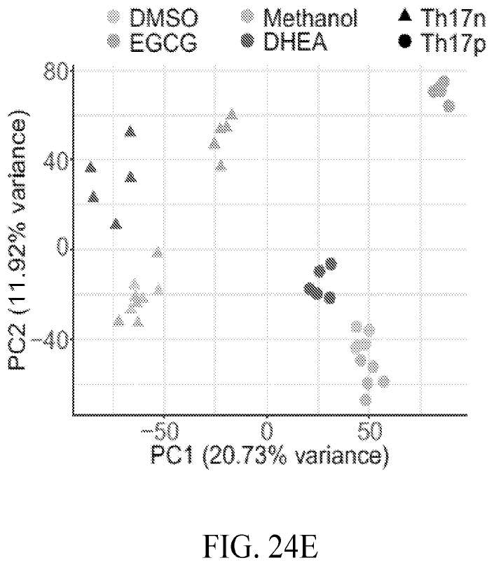

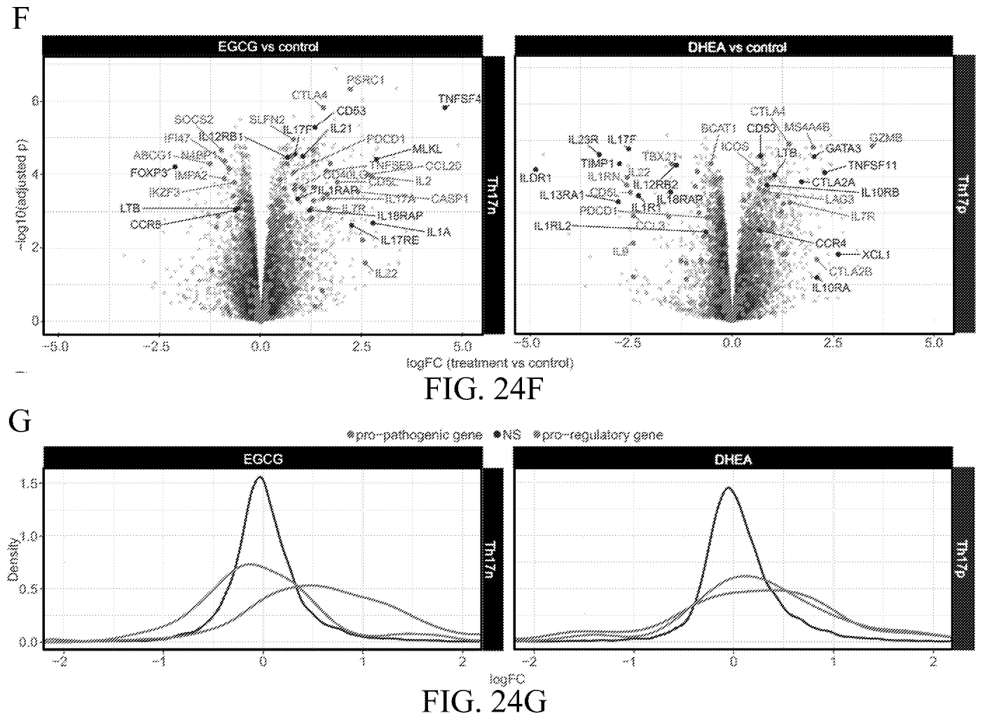

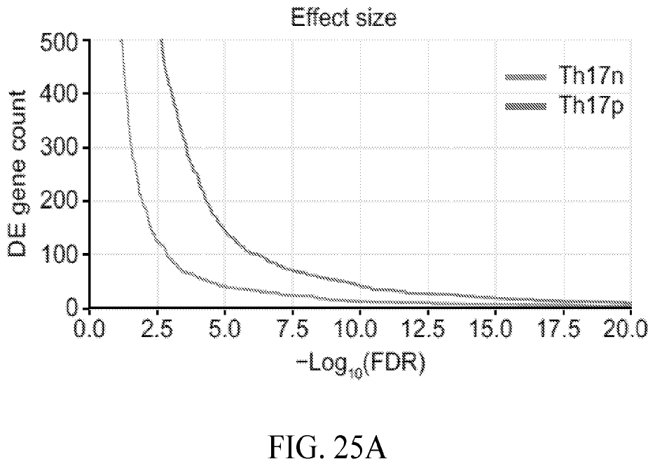

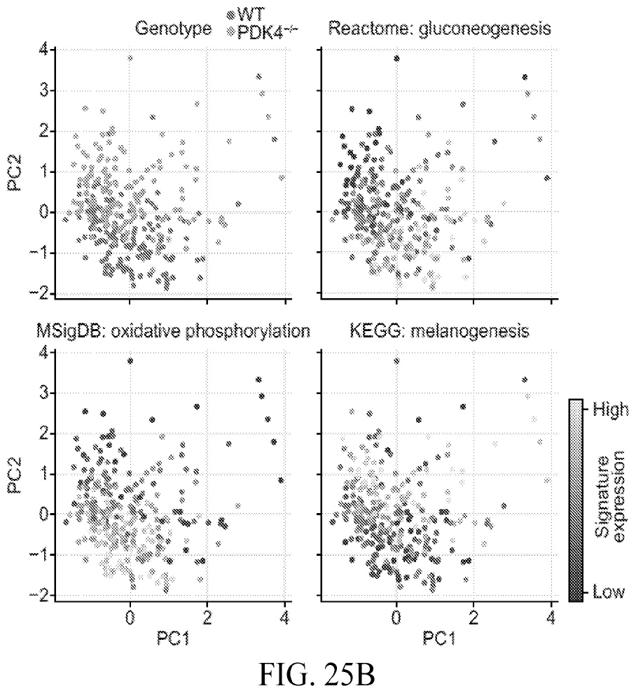

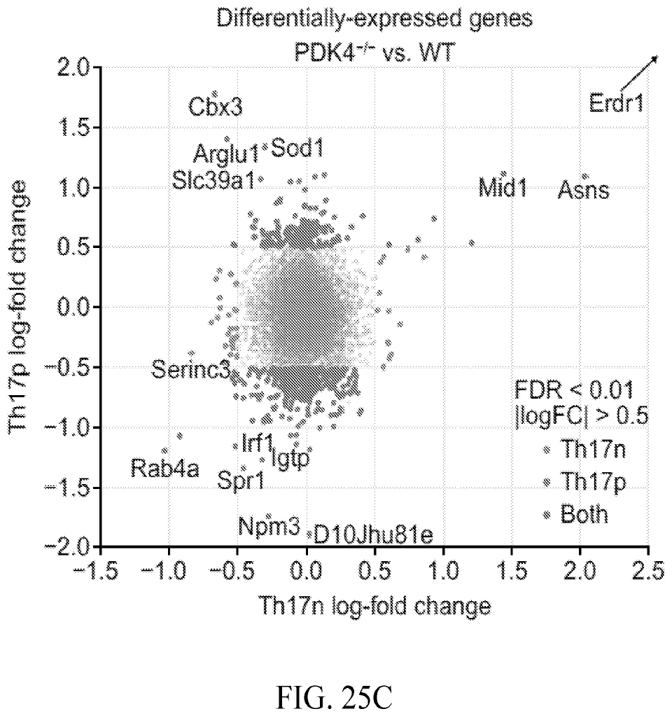

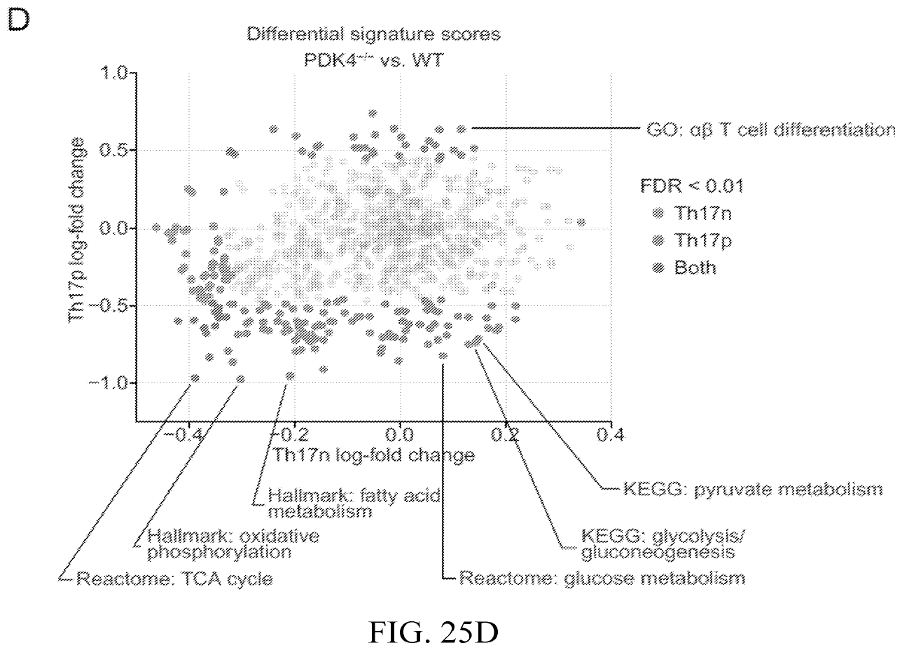

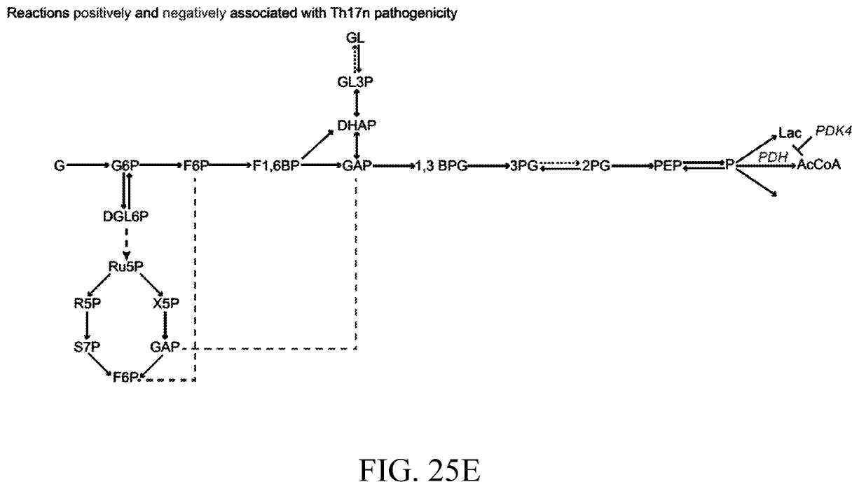

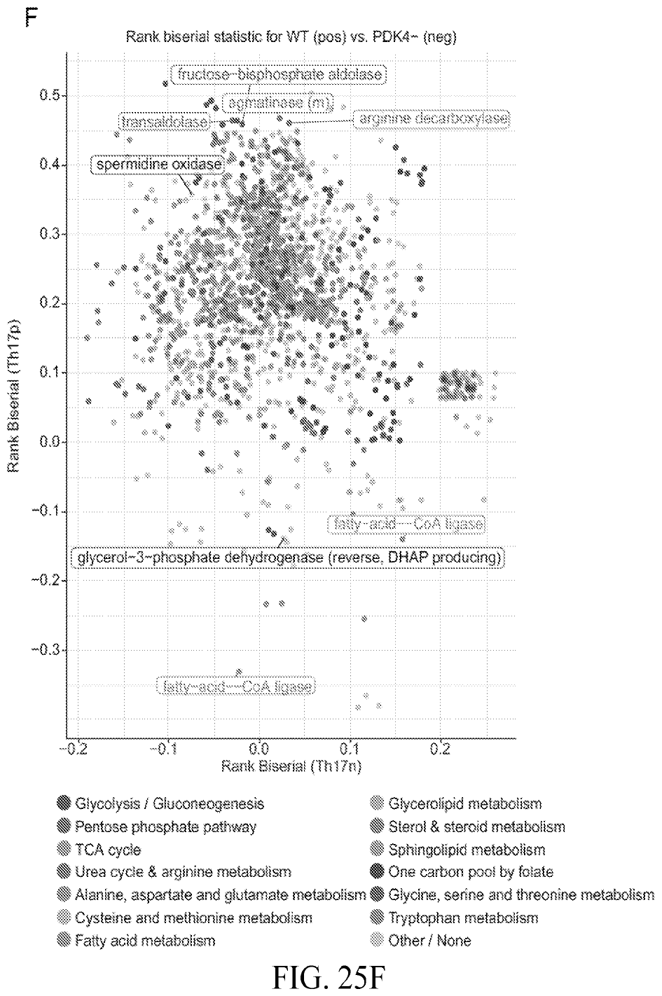

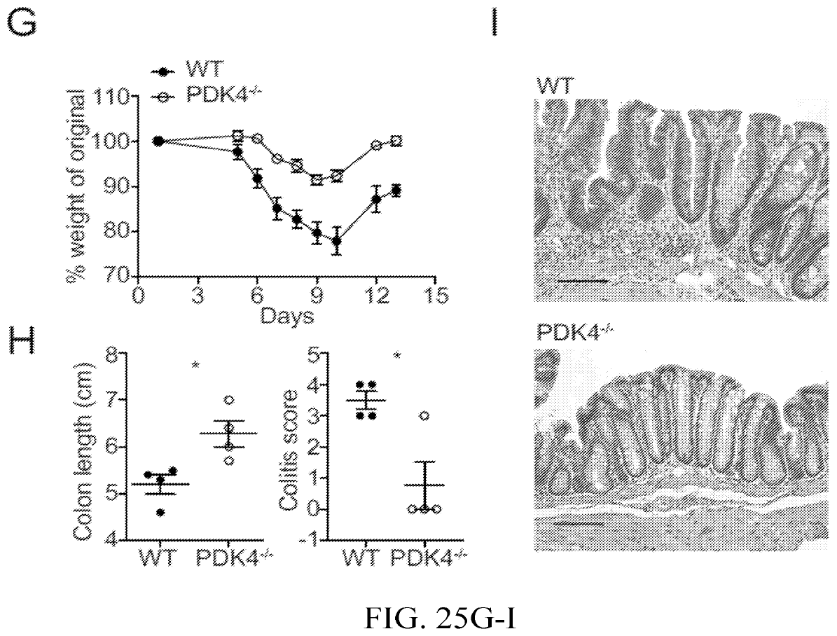

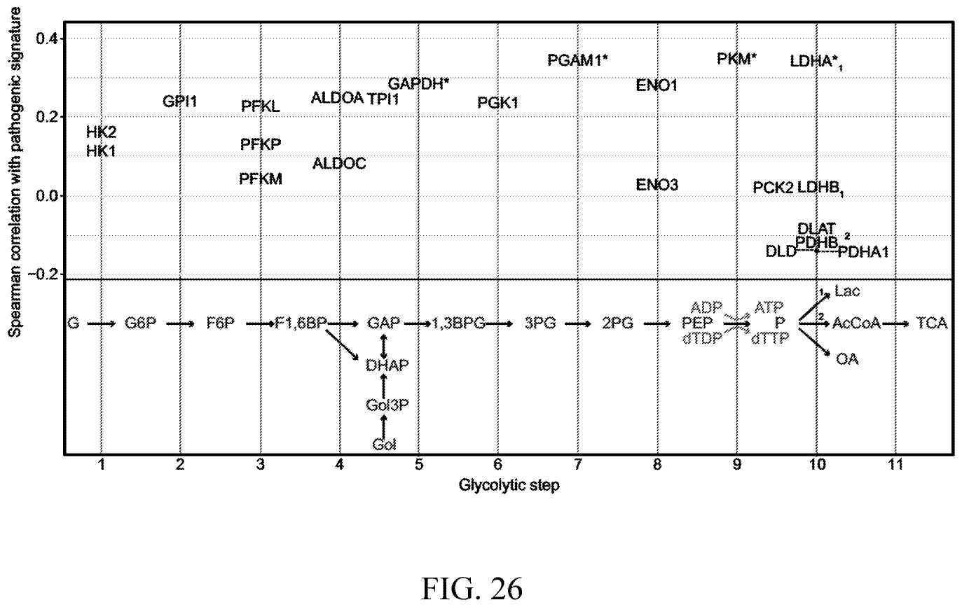

Applicants used COMPASS to identify additional pathways associated with Th17 pathogenicity. COMPASS predicted reactions in the glycolysis pathway that were positively and negatively associated with Th17 pathogenicity (FIG. 23E; Table 1 and 2). Applicants further validated glycolysis pathways with Th17 pathogenicity (FIG. 25B-D). FIGS. 24A and 26 shows the glycolysis reactions positively and negatively correlated with pathogenicity in non-pathogenic Th17 cells. The top positively associated genes are G6PD, PKM, PKM, G6PD, Aldo, PFKM, TA and G6PC. The top negatively correlated genes are PGAM, GK, PCK1, GK, ENO1, PCK1, TPI1, PGK1, GAPDHS, PGK1, PDHA1, GPD1 and GPD1. The genes are also shown in the pathway with inhibitors of each enzyme. The inhibitors shown may be used to alter the balance of Th17 pathogenicity in vitro and in vivo. Inhibitors of genes positively associated with pathogenicity can be used to shift Th17 cells away from pathogenic Th17 cells. Non-limiting inhibitors can be 2,5-Anhydro-D-glucitol-1,6-diphosphate, S-HD-CoA, DHEA, TX1, Gimeracil, Shikonin, or Pyruvate Kinase Inhibitor III. Inhibitors of genes negatively associated with pathogenicity can be used to shift Th17 cells towards pathogenic Th17 cells. Non-limiting inhibitors can be (+/−)2,3-Dihydroxypropyl dichloroacetate (DCA), 2,9-Dimethyl-BC, Koningic acid, CBR-470-1, EGCG, SF2312, PhAh, ENOblock, 3-MPA, or 6,8-Bis(benzylthio)octanoic acid. Dosages of inhibitors can be determined by one skilled in the art.

Applicants validated four of the reactions associated with pathogenicity of Th17 cells in the glycolysis pathway using inhibitors for each (FIG. 24B). Applicants differentiated naïve T cells under non-pathogenic or pathogenic Th17 conditions and treated the cells with control vehicle or the indicated drug. The cells were then analyzed by FACS for IL-17 and IL-2 positive CD4 T cells. The cells were allowed to divide only once during treatment, so the changes in positive cells was not due to cell death. G6PD2 positively correlated with pathogenicity and inhibition by DHEA resulted in a decrease in IL-17 positive CD4 T cells. PKM positively correlated with pathogenicity and inhibition by Shikonin resulted in a decrease in IL-17 positive CD4 T cells. PGAM negatively correlated with pathogenicity and inhibition by EGCG resulted in an increase in IL-17 positive CD4 T cells. GK negatively correlated with pathogenicity and inhibition by DCA resulted in an increase in IL-17 positive CD4 T cells.

### Example 7—in Silico Modeling of Metabolic Activity in Single Th17 Cells Reveals Novel Regulators of Autoimmunity

Cellular metabolism is both a mediator and a regulator of cell functions. Metabolic alterations are key in healthy cellular processes, such as differentiation, but also in diseases among which are cancer and ageing. Recently, the study of metabolism in immune cells (immunometabolism) has gained particular attention as the significance of intracellular metabolic phenotypes in viral-specific responses, autoimmunity, and cancer immunotherapy became evident¹⁻⁴.

The rapid advances in single-cell RNA-Sequencing (scRNA-Seq) enable novel ways of exploring the role of metabolism in health and disease, namely by studying cell-to-cell metabolic heterogeneity. In a recent review⁵, Applicants suggested that a cell's molecular contents, as measured by scRNA-Seq, for example, are the product of the instantaneous intersection of multiple biological factors, or vectors, that affected the cell. Specialized computational methods are needed to glean the unique information that can be inferred from single-cell data, while overcoming its challenges, such as sparsity due to dropout.

Here, Applicants address this challenge in the realm of cellular metabolism. Applicants present Compass, a novel algorithm to characterize and interpret the metabolic heterogeneity among cells in a quantitative and unsupervised manner. Compass belongs to the family of Flux Balance Analysis (FBA) algorithms⁶⁻⁸. It leverages a priori knowledge on the metabolic network's topology and stoichiometry in combination with the single-cell resolution and statistical power afforded by scRNA-Seq to map cell-to-cell metabolic heterogeneity and discover metabolic correlates of phenotypes of interest.

To demonstrate Compass's utility, Applicants analyze data from murine T helper 17 (Th17) cells, which is a heterogeneous cell type. On the one hand, Th17 cells are potent inducers of tissue inflammation in autoimmune disorders, among which are multiple sclerosis (MS) and inflammatory bowel disease (IBD)^{9,10}. On the other hand, they can play a protective role in promoting gut homeostasis and barrier functions^{11,12} Importantly, their effector functions, similar to those of other CD4+ and CD8+ T cell subsets are tightly linked to their metabolic state¹³⁻¹⁸. For these reasons, Th17 metabolism presents compelling questions that can be addressed via scRNA-Seq and Compass analyses.

Applicants show that Compass predicts known and novel associations between Th17 cells' metabolic state and their pathogenicity. Th17 pathogenicity is their capability to trigger autoimmune disease, which Applicants quantify with a transcriptomic (non-metabolic) signature ¹⁹. Applicants demonstrate both inter-group and intra-group analysis, i.e., both a comparative analysis of differences between two Th17 differentiation protocols, and an association study within a seemingly homogenous group of cells that were all differentiated using the same protocol. Notably, while previous studies in Th17^{16,20} as well as other immune cells²¹⁻²⁴ have all linked higher glycolytic activity with a pro-inflammatory cell state, Compass predicts, and subsequent assays validated, that phosphoglycerate mutase (PGAM)—a central reaction on the glycolysis path—is negatively associated with Th17 pathogenicity.

### Abbreviations

metabolites. G=glucose; G6P=glucose 6-phospohate; F6P=fructose 6-phosphate; F1,6BP=fructose 1,6-biphosphate; GAP=glyceraldehyde 3-phosphate; DHAP=dihydroxyacetone phosphate (also called glycerone phosphate); 1,3 BPG=1,3-biphosphoglycerate; 3PG=3-phosphoglycerate; 2PG=2-phosphoglycerate; PEP=phosphoenolpyruvate; P=pyruvate; Lac=lactate; AcCoA=acetyl-CoA; OA=oxaloacetate; TCA=tricarboxylic acid cycle; GL3P=sn-Glycerol 3-phosphate; GL=glycerol; DGL6P=D-glucono-1,5-lactone 6-phosphate; Ru5P=ribulose 5-phosphate; R5P=ribose-5-phosphate; X₅P=xylulose 5-phosphate; S7P=sedoheptulose 7-phosphate;

reactions. G6PD=glucose 6-phosphate dehydrogenase (EC 1.1.1.49); PKM=pyruvate kinase (EC 2.7.1.40); PGAM=phosphoglycerate mutase (EC 5.4.2.1); GK=glycerol kinase (EC. 2.7.1.30); KYAT=kynurenine-oxoglutarate transaminase (EC 2.6.1.7); ALTA=alanine transaminase (EC 2.6.1.2); GDH=glycerol dehydrogenase (EC 1.1.1.72); SPT=serine-pyruvate transaminase (EC 2.6.1.51);

inhibitors. DHEA=dehydroepiandrosterone; EGCG=epigallocatechin-3-gallate; DCA=3-dihydroxypropyl 2,2-dichloroacetate)

### Compass—an Algorithm for Comprehensive Characterization of Single-Cell Metabolism

Compass integrates scRNA-Seq profiles with prior knowledge of the metabolic network to infer a cell's metabolic state (FIG. 20a). The metabolic network is encoded in a genome-scale metabolic model (GEM) that includes the network's stoichiometry, biochemical constraints such as reaction irreversibility and nutrient availability, and gene-enzyme-reaction associations²⁵. To explore the metabolic capabilities of each cell, Compass solves a series of linear programs that produce a matrix of numeric scores, with rows corresponding to metabolic reactions and columns to cells (Methods). Intuitively, the score of a particular reaction in a particular cell is a proxy to the reaction's activity in that cell. This way, Compass represents cells as points in a high-dimensional metabolic space, whose coordinates denote putative activity of metabolic reactions, and is more readily interpretable in mechanistic terms than the high-dimensional gene expression space.

More rigorously, Compass scores reflect the propensity of cells to use certain reactions. Advances in scRNA-Seq provide scalable methods to count transcripts comprehensively and at a single-cell resolution^{26,27}, in ways that are not yet possible for other molecules, such as proteins. Therefore, studies often turn to gene expression in order to explore changes in cellular metabolic states. However, expression of a gene coding a certain enzyme do not always correlate with actual reaction flux^{28,29}, e.g., due to post transcriptional or post-translational modifications. Pathway-based analysis mitigates this concern by pooling information across genes and consequently enhancing robustness in the face of expression measurement noise, but it relies on a predetermined set of canonical metabolic pathways that do not fully capture the complexity of the metabolic network^{30,31}. Compass bridges this gap by using in silico modeling that helps determine which reactions are most likely promoted by the entire metabolic transcriptome. Further, Compass does not rely on predetermined pathway definitions, but derives metabolic pathways based on the observed data in an unsupervised manner.

Compass belongs to the family of Flux Balance Analysis (FBA) algorithms that model metabolic fluxes, namely the rate by which the substrates of a chemical reaction are converted to the reaction's products³². Its definition relies on a choice of an arbitrarily large set of arbitrary FBA objectives, which for simplicity Applicants defer to the Methods section, and instead describe a useful special case in which the objectives represent single-reactions. For each reaction, Compass determines the maximal flux it can carry, and then scores how well aligned is a cell's network-wide transcriptome with the objective of carrying that flux. Intuitively, Compass assumes that if the network-wide transcriptome of a particular cell supports carrying a large flux on a particular reaction, then this reaction is most likely active in the cell, even if its particular gene-coding enzyme is lowly expressed. Thus, a score reflects the propensity of a particular cell to use a particular reactions, which Applicants interpret as a proxy to the activity level of that reaction in that cell.

The framework allows formulating the aforementioned computation as a linear program and solving it efficiently. Like GIMME³³, Compass penalizes reactions inversely to the expression of mRNA associated with their enzymes (making the simplistic, yet common modeling assumption³⁴ that mRNA levels correlate with enzymatic activity). The compass score c_{r,i} of reaction r in cell i is the minimal network-level penalty subject to constraining the GEM of i to carry its maximal possible flux through r (up to a multiplicative slack factor). It therefore reflective of how well aligned is the transcriptome of cell i with the objective of carrying high flux through r.

Compass leverages the statistical power afforded by the large number of observations (i.e., single cells) in a typical scRNA-Seq study. This power allows downstream analysis to gain biological insight despite the high dimension of the metabolic space in which Compass embeds cells. However, scRNA-Seq presents unique challenges due to the small quantity of RNA that can be extracted from a single cell⁵. Sampling bias and transcription stochasticity lead to an abundance of dropouts, i.e., false-negative gene detections, and to variance overestimation of lowly expressed genes, leading in turn to false-positive differential expression. Similar to other scRNA-Seq algorithms, Compass mitigates these effects with an information-sharing approach³⁵⁻³⁷. Instead of treating each cell in isolation, the flux vector for each cell is determined by balancing its own gene expression with that of its k-nearest neighbors based on similarity of their RNA profiles (Methods).

### Data-Driven Prediction of Metabolic Targets Modulating Th17 Pathogenicity

Th17 functional diversity can be studied in vitro by polarizing them with either IL-1β+1L-6+1L-23 or TGF-β1+IL-6, which upon adoptive transfer into wildtype mice lead to severe or mild-to-none experimental autoimmune encephalomyelitis (EAE), respectively^{38,39} Applicants name those states “pathogenic” (Th17p) and “non-pathogenic” (Th17n), respectively. As described in ref. ¹⁹ Applicants sequenced CD4+naïve T cells 48 hrs post polarization under one of these conditions, ultimately retaining after quality tests 130 unsorted Th17n cells (henceforth Th17nu), 151 IL-17A/GFP+Th17n cells, and 139 IL-17A/GFP+Th17p cells. In this study, Applicants analyze the unsorted and sorted cells independently from one another. The unsorted cells are used to discover cell-to-cell heterogeneity within a seemingly homogenous population, whereas the sorted cell will be used for comparative study of the two polarizing conditions.

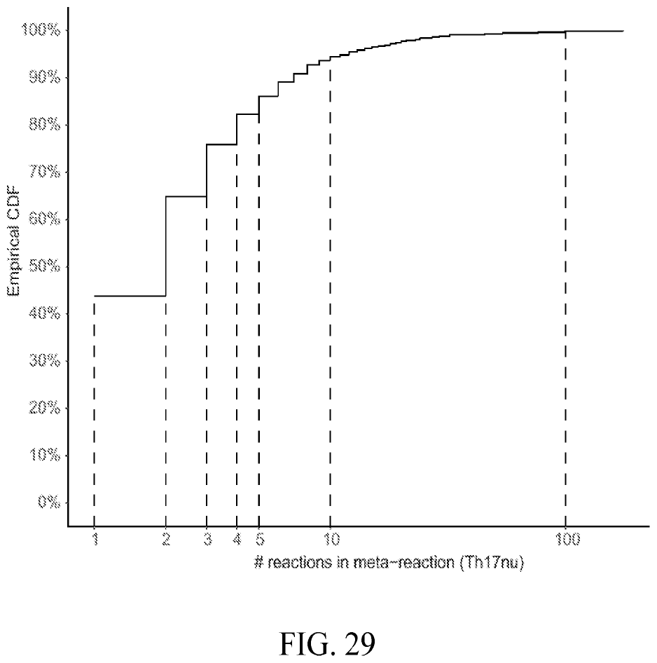

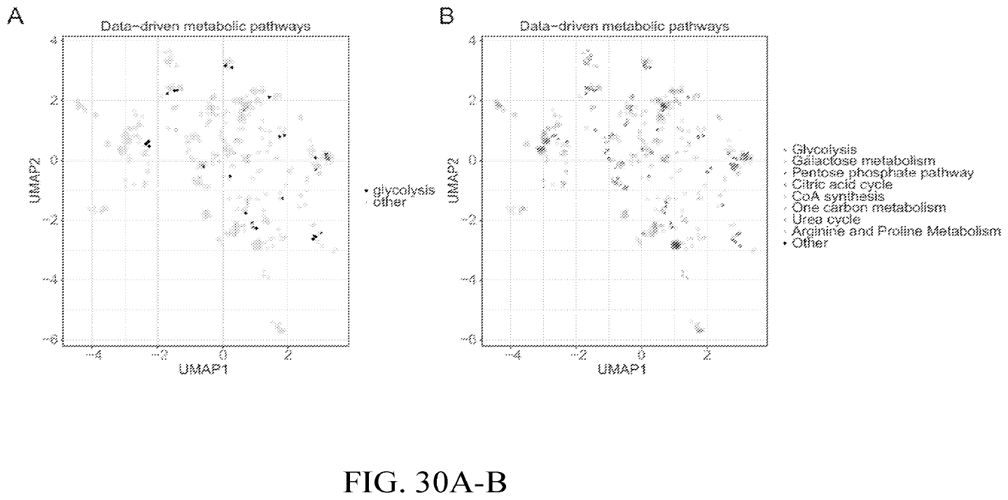

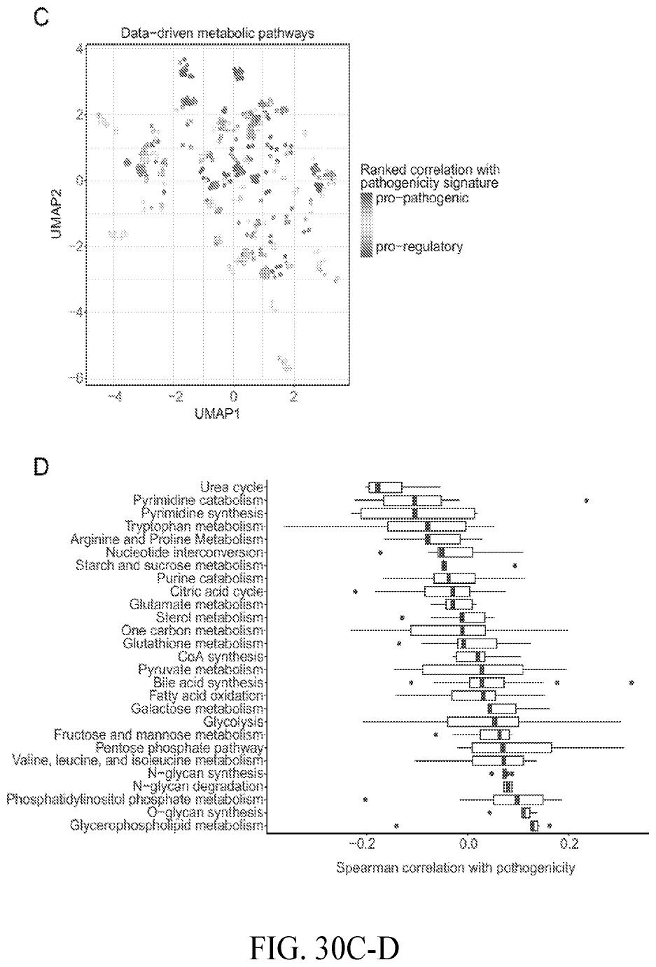

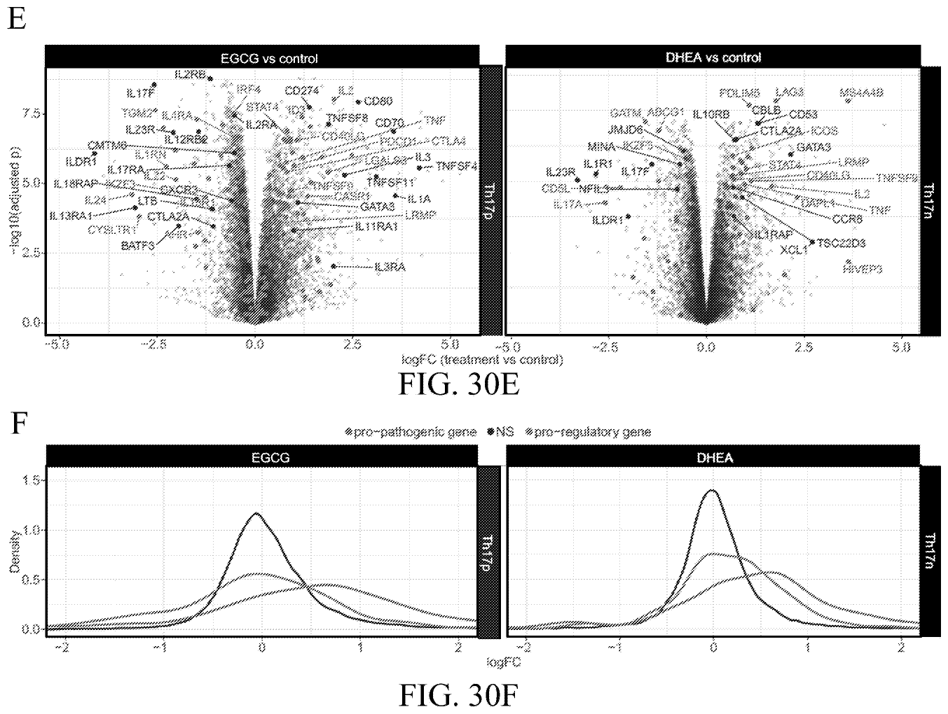

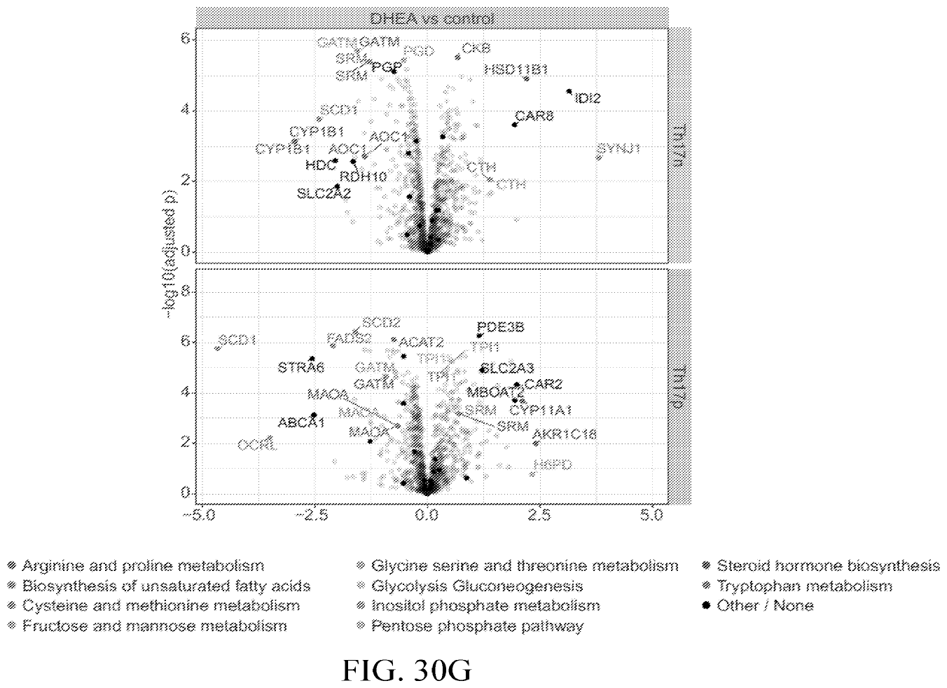

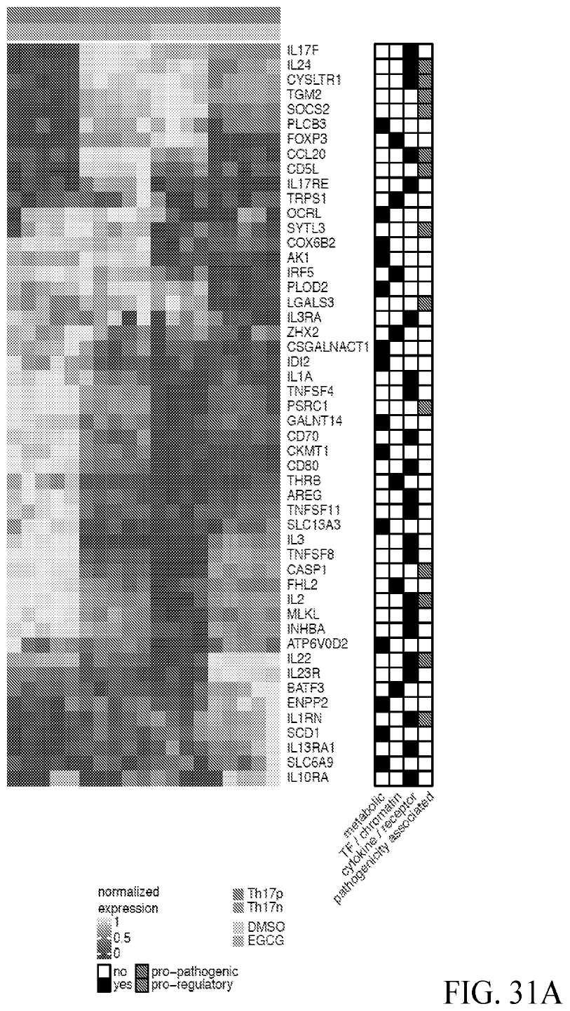

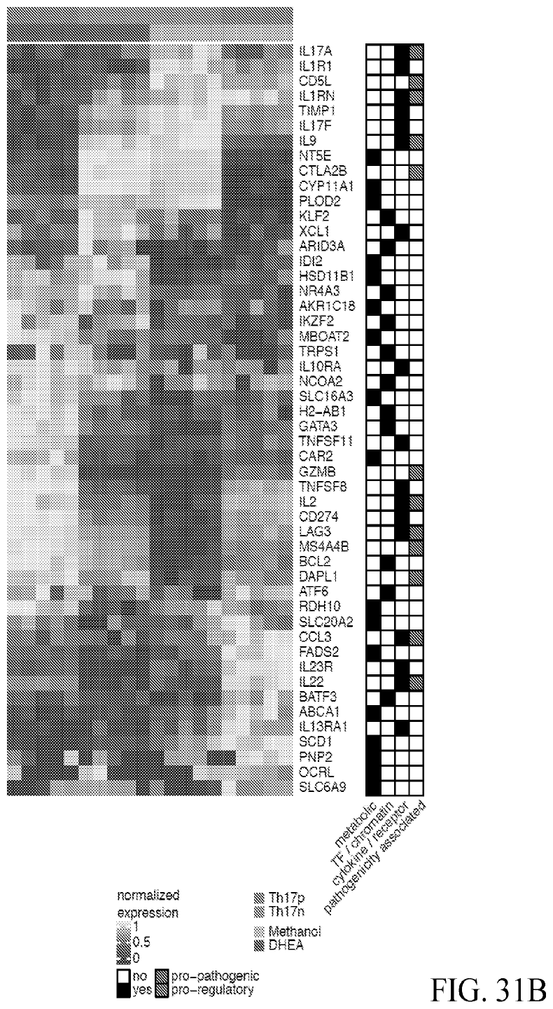

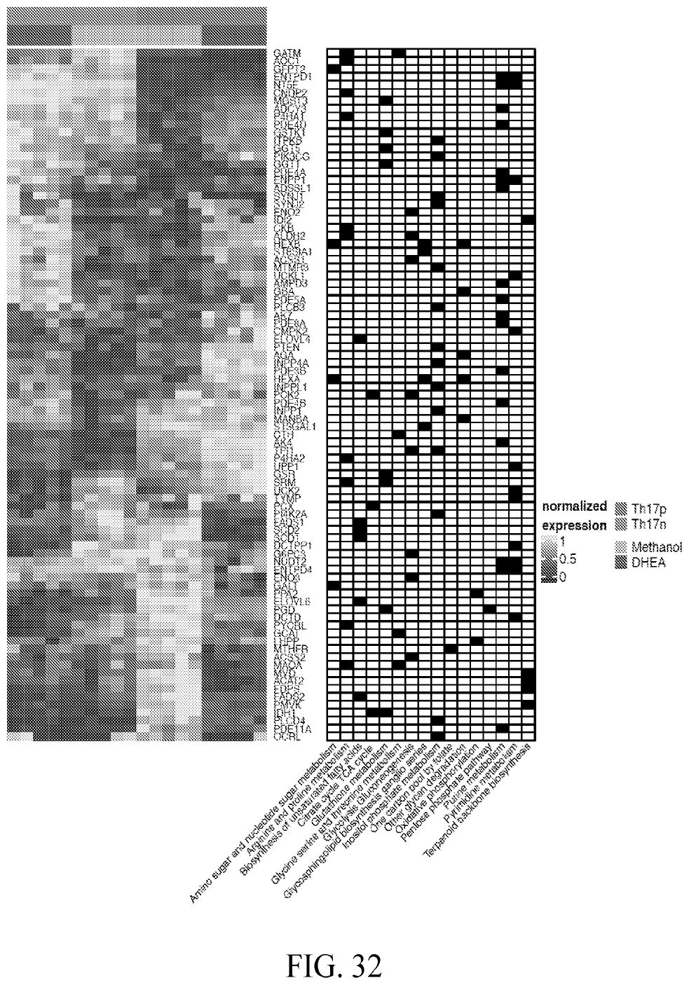

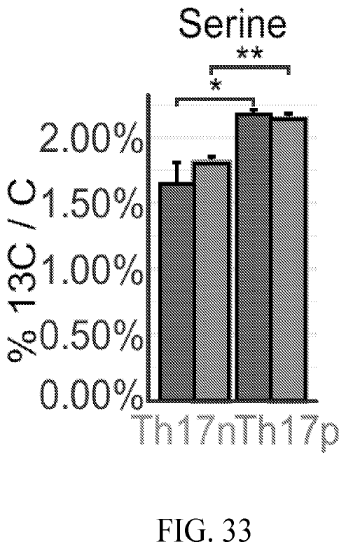

Beginning with the unsorted Th17nu population, Applicants computed the compass score for each metabolic reaction in each of the cells (Methods), producing a compass-score matrix of 6,563 reactions X 130 cells. Applicants hierarchically clustered the reactions (i.e., rows of the matrix) and merged reactions that were highly correlated across the entire dataset (Spearman rho≥0.98) into meta-reactions. This resulted in a compass-score matrix of 1730 meta-reactions X 130 cells, and with 76% of the meta-reaction composed of 3 reactions or less (FIG. 29). The aggregation step of reactions to meta-reactions facilitates analysis without obstructing biological interpretability of the results. Importantly, unlike common metabolic reaction sets, the meta-reactions are data-driven and may change between biological contexts.

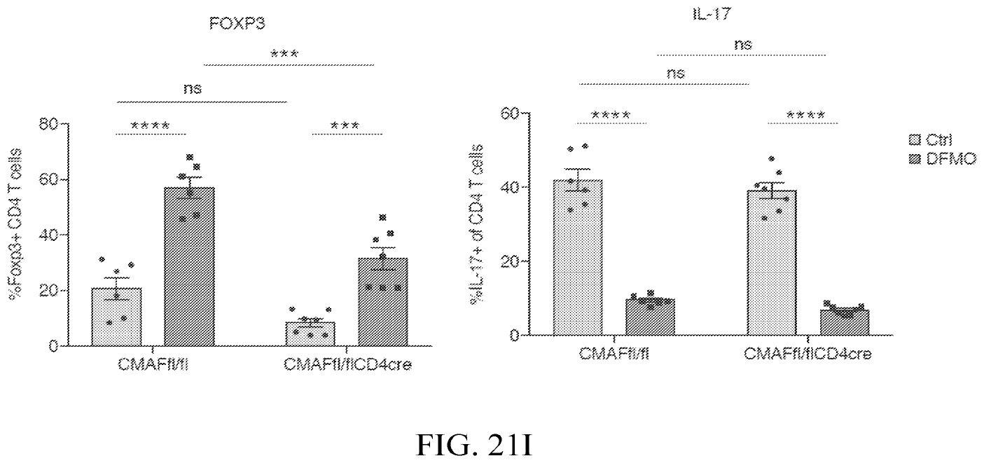

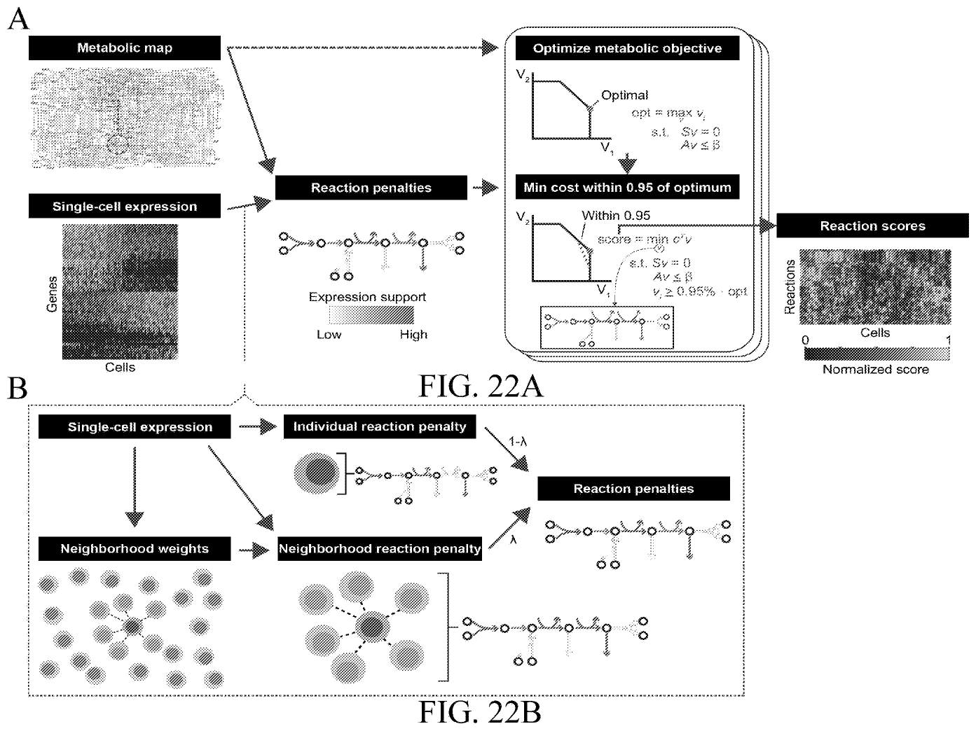

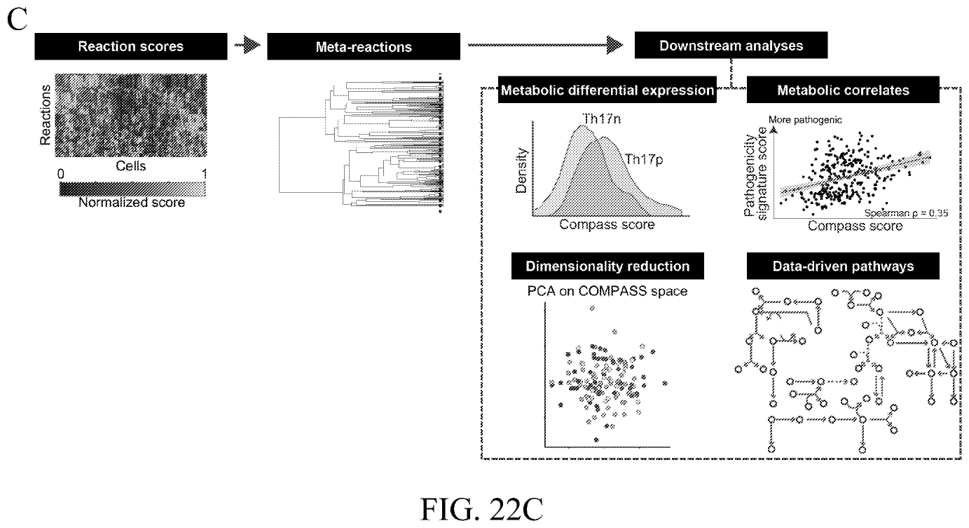

Gene expression analysis treat cells as points in a high-dimensional vector whose coordinates correspond to genes (or transcripts). Similarly, the Compass output allows studying the cells in a high-dimensional metabolic space whose coordinates correspond to meta-reactions. The first principal component (PC) of the metabolic (Compass) space corresponded to the cell's metabolic activity (FIG. 22B), defined as the ratio of a cell's transcriptome dedicated to metabolic genes. PC1 also highly correlated with a transcriptomic signature derived from Th17 differentiation time course⁴⁰. PC2 loadings indicated it corresponds to a metabolic axis pertaining to ATP generation strategy (FIG. 23C), with the negative side corresponding to cells engaged in aerobic glycolysis, and the positive side to cells that catabolize fatty-acids (beta-oxidation). Accordingly, PC2 coordinates were most anti-correlated with the GLUT3 and GLUT1 glucose transporters, and most correlated with CPT1A which codes a crucial enzyme for beta-oxidation (Spearman rho=−0.40, −0.37, +0.52, respectively, adjusted p<0.05 for all).

To predict metabolic regulators of Th17 pathogenicity, Applicants ranked metabolic reactions according to their correlation with a computational transcriptome signature of Th17 pathogenicity in Th17nu and Th17n (FIG. 22C, Methods). Reassuringly, the positive and negative ends of the ranked list recovered targets that are known to promote and suppress Th17 effector functions, respectively. For example, Compass indicated that reactions along the glycolytic pathway (with the exceptions discussed below) correlated with the pro-pathogenic phenotype, whereas tryptophan catabolism through the kynurenine pathway correlated with a pro-regulatory behavior. Indeed, aerobic glycolysis is considered a hallmark of activation in Th17 cells and other immune cell type, whereas the kynurenine pathway was shown to favor Treg differentiation over Th17⁴¹⁻⁴³ The analysis also suggested novel metabolic mediators of the Th17 pro-pathogenic or pro-regulatory behavior. One of the top novel predictions was that polyamine metabolism is correlated with the pro-regulatory phenotype. Applicants follow up on it in an accompanying manuscript.

Surprisingly, not all glycolytic reactions were correlated with Th17 pathogenicity. To test this prediction, Applicants picked the top two reactions that were most positively correlated and top two that were most negatively correlated with the pathogenicity score (FIG. 24C) and inhibited them in vitro with enzymatic inhibitors. Due to the possibly deleterious effects of knockouts on a central pathway on cell viability, Applicants restricted all analyses to cells that had undergone one division (dl) so as to exclude arrested or otherwise compromised cells. In addition, since two different solvents (DMSO and methanol) were needed for different inhibitors, every treatment group was matched with an appropriate vehicle control (Methods). Applicants found that IL-17 expression conformed to the computational prediction. It was significantly upregulated by the two inhibitors predicted to suppress pathogenicity, and downregulated by the two inhibitors predicted to promote pathogenicity (FIG. 24D). Importantly, EGCG and DCA treated cells retained their cytokine profile (FIG. 24E) indicating that they retained their Th17 identity while obtaining the pro-pathogenic phenotype, as elaborated below. In contrast, DHEA and shikonin curtailed cytokine production suggesting that their pro-regulatory phenotype is mediated through overall suppression of T effector functions.

Applicants proceeded to sequence RNA libraries from Th17n and Th17p under two inhibitors, DHEA and EGCG whose corresponding reactions were predicted to be the most pro- and anti-pathogenic, and were indeed found to significantly suppress or promote IL-17 expression, respectively. A PCA analysis of gene expression confirmed the validity of the dataset (FIG. 24E). First, the difference between the two vehicles was inconsequential compared to cell type and interventions. Second, PC1, which represents the main axis of variation in the data, represented as expected the pathogenicity phenotype. The location of the experimental groups with respect to PC1 accords with Compass's prediction and suggests that EGCG alters Th17n program toward a more pathogenic one, and DHEA suppresses the pathogenic program of Th17p. Differential expression analysis further supported this conclusion (FIG. 24F). DHEA led to over 3-fold decrease in IL-23R transcripts in both Th17p and Th17n and a 1-fold decrease in TBX21 (Tbet) transcripts in Th17p. More interestingly, EGCG strengthened the pro-pathogenic transcriptional program in Th17n, upregulating across the board pro-pathogenic genes and (to a more limited extent) down-regulating pro-regulatory ones (Methods). For example, IL22, IL7R, and CASP1 were up-regulated whereas IKZF3 was downregulated. However, Applicants note that SGK1 and CD5L behave opposite to their expected direction.

In conclusion, Compass correctly predicted metabolic targets whose deletion affected Th17 pathogenicity. Importantly, it was able to pinpoint a glycolytic reaction that suppresses Th17 pathogenicity, which runs contrary to the ubiquitous observation that aerobic glycolysis is associated with an activated T cell state.

### Data-Driven Discovery of Differential Metabolic Programs in Pathogenic and Non-Pathogenic Th17

The division of the metabolic network into functional pathway, namely groups of topologically adjacent reactions thought to operate coherently, is indispensable in the study of metabolism. Nonetheless, the canonical textbook pathways may not translate between different cellular environments, between organisms, or between healthy and disease (e.g., cancerous) cells. Carbon tracing studies have already observed metabolic flows that are contrary to long-standing beliefs⁴⁴, as well as inter-cellular division of labor across canonical pathways⁴⁵.

Applicants therefore suggest data-driven metabolic pathways as a valuable data-exploratory tool. Learning pathways from the data affords complementary strengths to the ubiquitously employed enrichment analysis versus collections of a priori defined pathways, such as KEGG or MetaCyc. Previous studies have suggested this concept^{31,46} but did not benefit from the statistical power of scRNA-Seq available through Compass.

To find data-driven pathways in a given set of cells, Applicants define the distance between metabolic reactions based on cosine dissimilarity of their Compass profiles across the set, and use it to construct a k-nearest neighbor (kNN) graph over the set of metabolic reactions (Methods). Communities in the graph, found for example by the Louvain algorithm, are defined as the data-driven pathways.

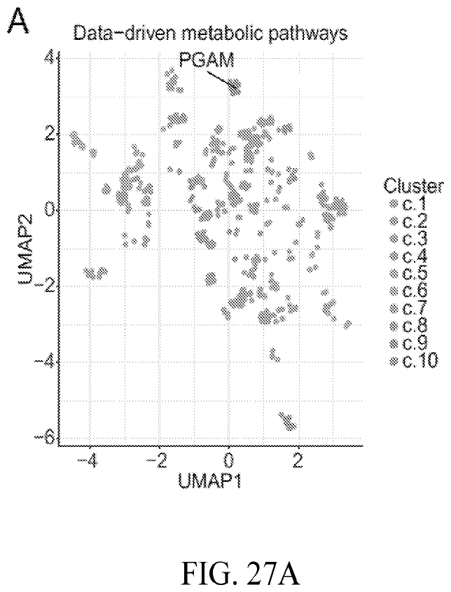

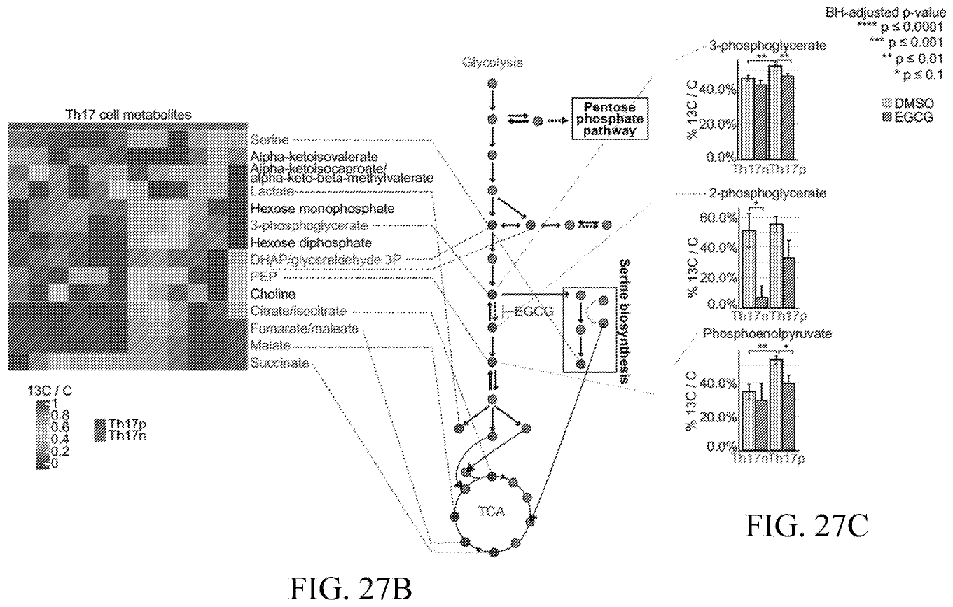

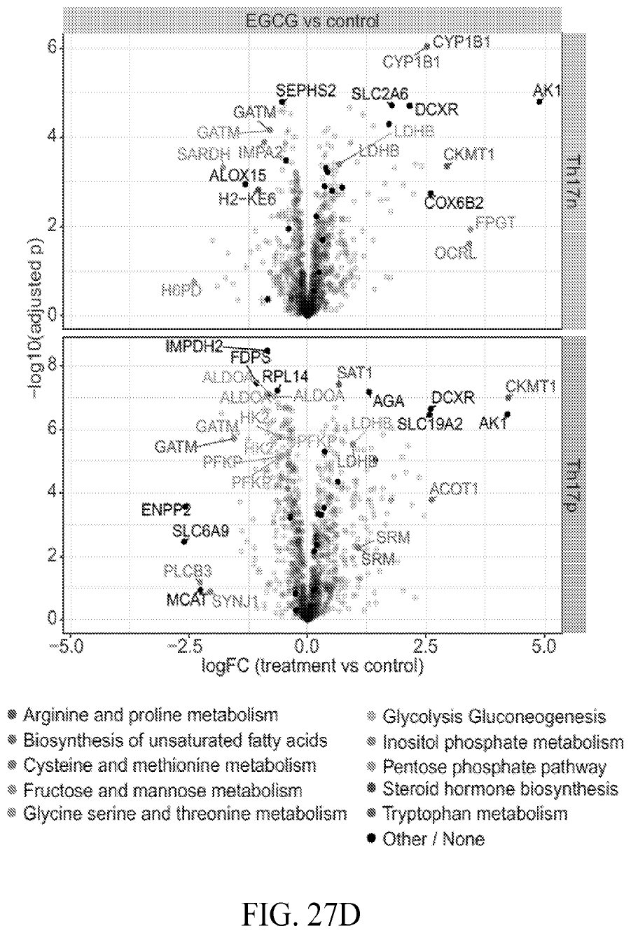

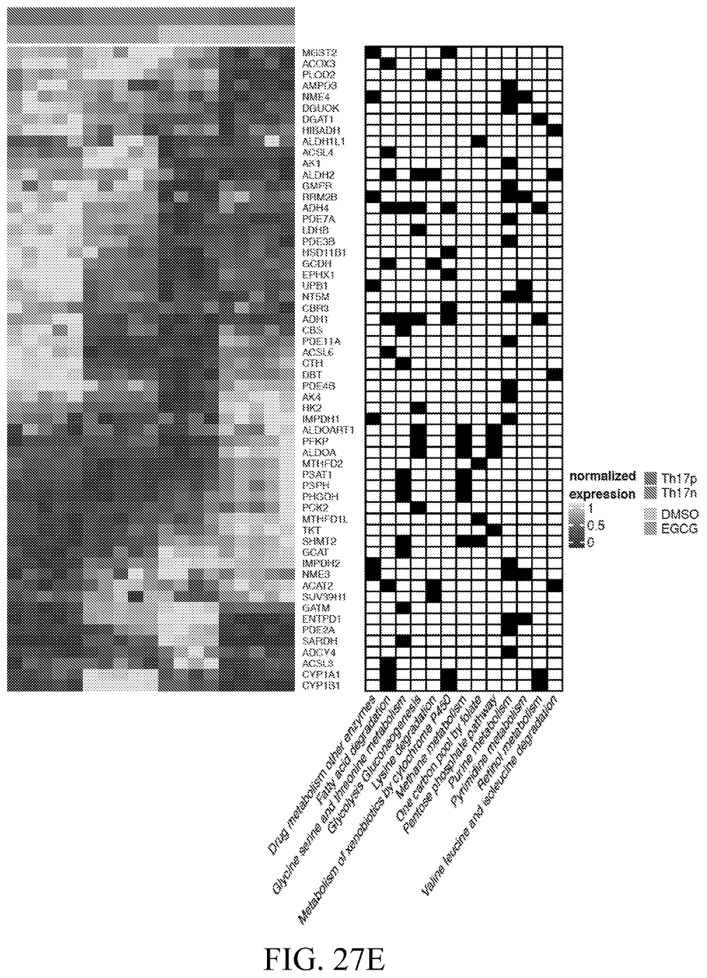

Applicants applied this procedure separately to Th17n and Th17p cells to learn the difference in their metabolic rewiring in an unsupervised manner (FIG. 27A). Interestingly, the PGAM reaction which was predicted to promote the pathogenic behavior in Th17n belonged to different pathways in Th17n vis-a-vis Th17p. In Th17p PGAM was clustered with upstream glycolytic reactions, with lactate dehydrogenase, and with glycerolipid as well as fatty-acid synthesis, all of which correspond to metabolic phenotypes distinguishing Th17 from Tregs. In Th17n, however, it clustered with its downstream PEP and PCK reactions, as well as multiple TCA cycle reactions. To test this observation, Applicants conducted a carbon tracing assay in which the cell's medium was supplemented with 13C-glucose (Methods). Indeed, Th17n differentially shunted glucose-derived carbon into the TCA cycle, whereas in Th17p the 13C was over-abundant in glycolytic intermediates and byproducts of alternative carbon fates, other than TCA, that fork from glycolysis (FIG. 27). PGAM inhibition with EGCG led to a decrease in 13C contents of 3PG, 2PG and PEP (upstream glycolytic metabolites could not be detected in this assay) in both Th17n and Th17p as might be expected from a glycolytic inhibitor. However, the depletion of glucose-derived carbon in 2PG was most striking in 2PG and even more so in Th17n (from ˜50% 13C ratio to −10%) than in Th17p (FIG. 27B). This differential effect of PGAM inhibition on the metabolic phenotype was apparent also in RNA-Seq. Several glycolytic genes upstream of PGAM were downregulated in Th17p, but not Th17n, subjected to PGAM inhibition as well as multiple genes involved in serine biosynthesis (forking from 3PG as an alternative fate to PGAM) and one-carbon metabolism.

### Studying the Inhibitors' Effect In Vivo

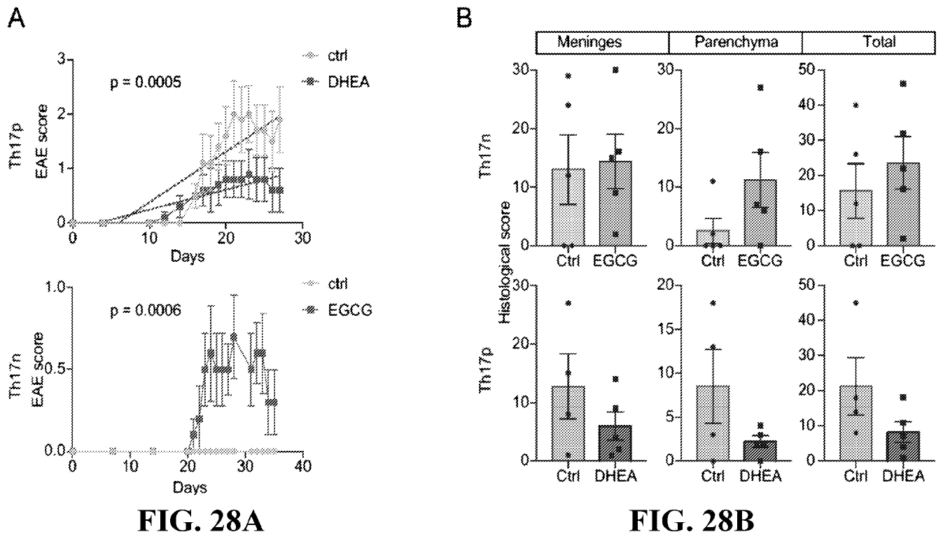

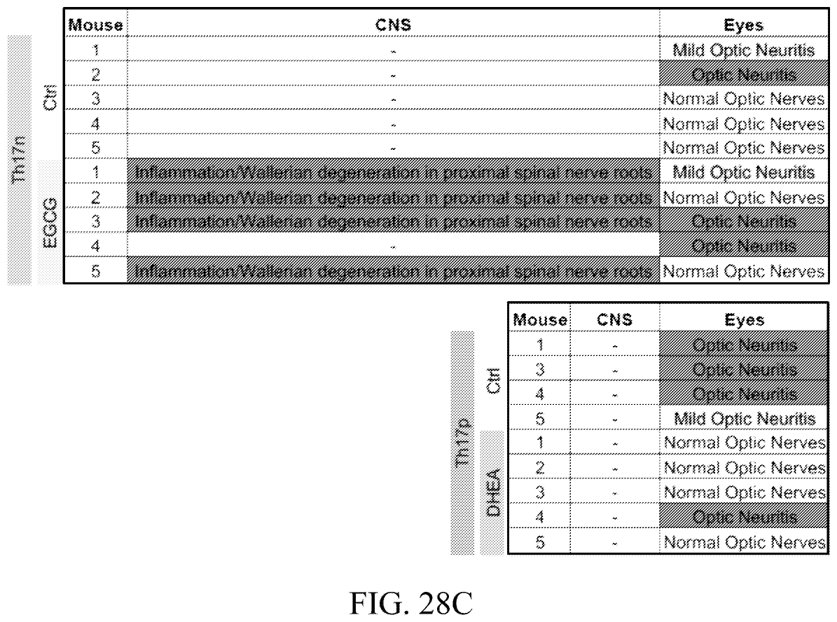

To study the inhibitors' effects in vivo, Applicants activated 2D2 TCR-transgenic Th17 cells in the presence of an inhibitor or vehicle and adoptively transferred them back to test animals. In accordance with computational predictions, EGCG-treated Th17n cells successfully induced EAE, whereas untreated cells failed to produce any consequential neuroinflammation (FIG. 28A-B). On the flip side, DHEA-treated Th17p induced a milder form of the disease compared to untreated Th17p. Interestingly, EGCG-treated Th17n were the only experimental group to produce Wallerian degeneration in proximal spinal nerve roots (FIG. 28C).

### Discussion

Applicants presented Compass—a flux balance algorithm for the study of metabolic heterogeneity among cells based on single-cell transcriptome profiles. The algorithm is applicable to any cell type whose transcriptome can be sequenced. Applicants used it to analyze a Th17 dataset and look for metabolic correlates of a transcriptomic pathogenicity signature. Compass correctly predicted a glycolytic reaction that, common to common understanding, promotes a pro-regulatory rather than a pro-inflammatory phenotype, as well as a pro-inflammatory role for the polyamine pathway that is studied in depth in an accompanying manuscript.

For computational tractability, static FBA algorithms assume that the system operates in chemical steady state (Varma and Palsson 1994). Even under this assumption, there remain an infinite number of feasible flux distributions—assignments of an activity level to each flux in the network—that satisfy the preset biochemical constraints (Methods). Therefore, most studies assume that the system (here, a cell) aims to optimize some metabolic function, usually production of biomass or ATP⁴⁷. However, whereas such objectives may successfully predict unicellular organisms' phenotypes⁴⁸, they are ill-suited for studying mammalian cells⁴⁹. To overcome this challenge, rather than optimizing a single metabolic objective function, Compass optimizes an arbitrarily large set of arbitrary objectives that together capture multiple facets of the cell's metabolic capabilities. The vector of optimal values obtainable in these objective represents a cell as a point in a space whose dimension is the set's size, which Applicants denote the Compass space. The set of objectives. A biological signal can be detected in the high-dimension owing to the statistical power afforded by the large number of sequenced libraries in a typical scRNA-Seq. Nonetheless, there is no obstacle preventing one from running Compass on bulk RNA data (typically while setting the parameter lambda to 0 to prevent information sharing between RNA libraries) as an exploratory analysis method.

The metabolic reconstruction Applicants employed represents the overall metabolic capabilities of a human cell. As such, it contains reactions that may not be available to the studied cell type—a concern that can be remedied to some extent by procedures for deriving organ-specific metabolic models (Opdam et al. 2017). Moreover, Applicants used the network to study murine data because no recent and equally validated reconstruction exists for mouse. Last, the metabolic profile of a cell depends on the nutrients available in its environment, which are often poorly characterized. The computations are based on a rich in silico environment, and modifying the latter to better represent physiological conditions should increase the algorithm's predictive capabilities.

### Example 7 Materials and Methods

Software. Compass is available at github.com/YosefLab/Compass

The algorithm is highly parallelizable. It currently supports execution on multiple threads in a single machine, submission to a Torque queue, and execution on a single machine on Amazon Web Services (AWS).

Compass algorithm. Compass transforms a gene expression matrix G, where rows represent genes and columns represent RNA libraries (usually, single cells) into a matrix C of Compass scores where rows represent metabolic reactions, columns are the same RNA libraries as in the gene expression, and an entry quantifies a proxy for reaction's activity level. More precisely, the entry quantifies the propensity of the cell to use that reaction, as formalized below.

For clarity purposes, Applicants provide here a high-level description, and defer exact formulation to the Supplementary Methods. Applicants slightly abuse notation in writing ƒ(M) for a matrix M=(m_{i,j}) and a function ƒ: [symbol] → [symbol] to denote the transformation ƒ(M): =(ƒ(m_{i,j})) where the intention is obvious from the context.

Select a genome-scale metabolic network (GEM) according to the dataset in question. Pick an arbitrary set of m linear objective function over the space of reaction flux distributions. Let p(r) be a monotonically decreasing penalty function defined on [0, ∞). Applicants used p(r): =1/(1+r). Let G be the input gene expression matrix. Only metabolic genes, i.e., ones annotated in the metabolic network, are used. The main steps are (FIG. 22):

Preprocessing: for computational tractability, the number of cells in G can be reduced by downsampling or, preferably, micropooling (see below).
1) Transform the gene expression matrix into a reaction expression matrix R, where rows represent single metabolic reactions, based on gene-reaction boolean relationships embedded in the GEM.
2) Compute a neighborhood reaction expression matrix R^{N} by replacing every cell's profile (column in R) with a weighted average of its neighborhood in the gene expression space.
3) Transform reaction expression to penalties for unevidenced reactions by applying p(r). Denote P: =p(R), P^{N}: =ₚ(R^{N}).
4) Compute a smooth penalty matrix \hat{P}: =(1−λ)·P+λ·P^{N}. A user-chosen parameter λ∈[0,1] controls the weight given to the cell's neighborhood. This mitigates the effects of technical noise, and importantly of dropouts, in scRNA-Seq data.
5) For each cell and each objective, solve two linear optimization programs:
   a) Find a flux distribution that maximizes the objective, subject to constraints imposed by the metabolic network (e.g., mass-balance). Let v^{opt} be the maximum value. Note that this step is independent of G and can be computed in advance and cached for computational efficiency.
   b) Find a flux distribution that minimizes the penalty due to poorly-evidenced reactions in R, subject to achieving the objective at a level of at least ω·v^{opt} (ω=0.95) in addition to the previous constraints.
6) Let n be the number of cells in G. Let C^{raw} be the m×n matrix obtained by taking the minimal penalties computed above.

Postprocessing: Normalize C^{raw}. Importantly, this step negates the matrix in order to transform the penalties into proxies for metabolic activity. It may also merge similar rows (objectives that resulted in similar profiles across the cells). The resulting m′×n (m′≤m) matrix C is the Compass matrix. C embeds the gene expression profiles in [symbol] ^{m′}.

Metabolic network and choice of objective functions. Applicants used the Recon2 GEM²⁵, which Applicants transformed to a unidirectional network by replacing bidirectional reactions with the respective pair of unidirectional reactions. Throughout this application, metabolic genes are defined as the set of genes annotated in Recon2.

The results of flux balance analysis significantly depend on the nutrients made available to the GEM, referred to as the in silico growth medium. Since exact medium composition is mostly unknown even for common in vitro protocols, and certainly unknown in vivo, Applicants chose a rich in silico medium where all nutrients are made available.

Applicants used an intuitive set of objective functions—for each reaction in the network, Applicants defined one objective function which is to maximize the flux it carries. This allows intuitive interpretation of the Compass scores as quantitative proxies to reaction activities. Some of these objective functions need not be computed in practice because their respective reactions are blocked, namely there exists no feasible solution in which they carry non-zero flux.

Preprocessing. For prohibitively large datasets, Applicants recommend using a micropooling approach⁵⁰ as implemented in the VISION R package (github.com/YosefLab/VISION), in which small numbers of similar cells are grouped together, their transcriptomic profiles are averaged, and the averaged profile is treated as a single cell in subsequent analysis. This was not necessary for the datasets presented in this application.

Smoothing and information sharing between cells. Applicants computed a k-nearest neighbors (kNN) graph (k=5) based on Euclidean cell-to-cell distances in a reduced-dimension (top 20 PCs) of the gene expression space. A cell's neighborhood for the purpose of computing R^{N} was its k neighbors. Compass also supports a Gaussian kernel smoothing instead of the kNN approach (Supplementary Methods).

Note that Compass easily accommodates bulk RNA data (i.e., standard RNA-Seq where libraries represent many cells) and microarrays by setting λ=0. Applicants chose λ=0.25 for single-cell datasets and λ=0 for bulk RNA libraries.

Postprocessing. Using the objective function defined above, every row in C^{raw} represents a penalty for maximizing or minimizing the flux on a certain unidirectional metabolic reaction. Applicants hierarchically clustered the rows by Spearman distance, and merged together leaves in which Spearman similarity (namely 1−ρ, with ρ being Spearman's correlation) by averaging the respective rows. Applicants call the resulting clusters meta-reactions and each represents a set of closely correlated metabolic reactions. Importantly, the division into meta-reactions is data-driven and does not rely on canonical metabolic pathway definitions (FIG. 22b). Therefore, the division is dataset-dependent—for example, two reactions might be closely correlated and clustered in the same meta-reaction in one cell type, but not in another.

Let C^{meta-raw} be the result of the merging step. By definition, all its entries are non-negative. Normalize it as follows:
a) Transform C^{meta-raw}: −log(1+C^{meta-raw})
b) Let C: =C^{meta-raw}−min(C^{meta-raw})i.e. decrease the smallest entry from each entry. All entries are not non-negative and represent a quantitative proxy for (meta-) reaction activity, rather than penalties.
c) Remove constant rows from C. Applicants removed rows where the difference between the largest and smallest score was less than 1e-3.

### C is the Compass Matrix Returned for Downstream Analyses.

Bulk RNA-Seq and SMART-Seq2 analysis. Applicants aligned single-cell SMART-Seq2 libraries with Bowtie2, quantified TPM gene expression with RSEM, and performed QC as Applicants described in detail in a previous publication⁵¹. This computational pipeline is a massively revised and updated version of the one originally used to analyze these libraries¹⁹. Batch effects and other nuisance factors were normalized with a model chosen empirically with SCONE ⁵². Bulk RNA-Seq were processed with a modified variant of the same pipeline. In the absence of UMIs, differentially expressed genes were called through a linear model fitted to TPM values with the limma R package and with a mean-variance trend added to the empirical bayes prior^{53,54}.

Mice. C57BL/6 wildtype mice (WT) were obtained from Jackson laboratory (Bar Harbor, Me.) (IL-17A.GFP, 2D2 mice PDK4). All experiments were approved by and carried out in accordance with guidelines of the Institutional Animal Care and Use Committee (IACUC) at Harvard Medical School.

T cell differentiation culture and Flow cytometry. Naïve CD4+CD44-CD62L+CD25− T cells with or without including IL-17A.GFP+ were sorted using BD FACSAria sorter and activated with plate-bound anti-CD3 and antiCD28 antibodies (each 1 μg/ml) in the presence of cytokines at a concentration of 5×10⁵ cells/ml. For T cell differentiations the following combinations of cytokines were used: pathogenic Th17 (Th17p): 25 ng/ml rmIL-6, 20 ng/ml rmIL-1b (both Miltenyi Biotec) and 20 ng/ml rmIL-23 (R&D systems); non-pathogenic Th17 (Th17n): 25 ng/ml rmIL-6 and 2 ng/ml of rhTGFb1 (Miltenyi Biotec). For differentiation experiments, cells were harvested at 72 hours and were performed in the presence or absence of 50 μM EGCG (Selleck Chemicals), 50 μM DHEA, 40 μM DCA, 10 μM Shikonin (all Sigma) as indicated.

Intracellular cytokine staining was performed after incubation for 4-6h with Cell Stimulation cocktail plus Golgi transport inhibitors (Thermo Fisher Scientific) using the BD Cytofix/Cytoperm buffer set (BD Biosciences) per manufacturer's instructions. Transcription factor staining was performed using the Foxp3/Transcription Factor Staining Buffer Set (eBioscience).

Proliferation was assessed by staining with CellTrace Violet (Thermo Fisher Scientific) per manufacturer's instructions. All stainings were analyzed on a FACS LSR II (BD Bioscience) and using FlowJo software.

Legendplex. Cytokine concentrations in supernatants of in vitro cultures were analyzed by the LegendPlex Mouse Th Cytokine Panel (13-plex) (BioLegend) according to the manufacturer's instructions and analyzed on a FACS LSR II (BD Biosciences).

Antibodies. All other flow cytometry antibodies were purchased from BioLegend.

Experimental Autoimmune Encephalomyelitis (EAE). For active EAE immunization, MOG35-55 peptide was emulsified in complete freund adjuvant (CFA). Equivalent of 40 μg MOG peptide was injected per mouse subcutaneously followed by pertussis toxin injection intravenously on day 0 and day 2 of immunization. Mice were monitored and assigned grades for clinical signs of EAE using the following scoring system: 0, healthy; 1, limp tail; 2, impaired righting reflex or ataxic gait; 3, hind limb paralysis; 4, total limb paralysis; 5, moribund or death. Mice with a score of >4 were euthanized. If mice died during the course of the experiment, their clinical score of 5 was included in the analysis for the remainder of the experiment.

For adoptive transfer EAE, naïve 2D2 transgenic T cells were sorted and differentiated into Th17n cells+/−EGCG or Th17p+/−DHEA as described for three days followed by a resting phase in the presence of IL-23 alone for 2 days. Cells were then harvested and restimulated with plate-bound anti-CD3 and anti-CD28 for 2 days prior to transfer. 2-8 million cells were transferred per mouse intravenously. EAE was scored as previously published (Jager et al., 2009) or as described above.

Metabolomics/Carbon tracing. For untargeted metabolomics, Th17 cells were differentiated as described. Culture media were snap frozen. Cells were harvested at 96h. 10×10⁶ cells per sample were snap frozen and extracted in either 80% methanol (for fatty acids and oxylipids) or isopropanol (for polar and nonpolar lipids). Two liquid chromatography tandem mass spectrometry (LC-MS) methods were used to measure fatty acids and lipids in cell extracts.

For carbon tracing experiments Th17 cells were differentiated as described. Thereafter, cells were washed and cultured in media supplemented with 8 mM [U-13C]-glucose for 15 min or 3 hrs.

Let |Cᵢ| be the number of carbon atoms in metabolite i, and let x_{c,i,j} be the measured signal of metabolite i in sample j (subsequent to all normalization and QC procedures) in which there are exactly c 13C atoms. The comparisons of carbon flows are based on the statistic y_{i,j}=(Σₜ₌₀^{|C}^{ᵢ}^{|}t·x_{t,i,j})/(|Cᵢ|·Σₜ₌₀^{|C}^{ᵢ}^{|}x_{t,i,j})

which measures the 13C ratio of the total carbon contents for metabolite i in sample j.

The Compass Algorithm

### Reaction Expression

The first step in Compass is to create the R matrix, which assigns, for each cell, an expression value to each metabolic reaction. This is done using the boolean gene-to-reaction mapping included in the selected GEM [put refs with similar methods].

If a single gene with linear-scale expression x is associated with the reaction, then the reaction's expression will be log₂(x+1). Units of x can be TPMs (as in this application), CPMs, or any other units chosen by the user.

Many reactions, however, are associated with multiple genes and this association is expressed as a Boolean relationship. For example, two genes which encode different subunits of a reaction's enzyme are associated using an AND relationship as both are required to be expressed for the reaction to be catalyzed. Alternately, if multiple enzymes can catalyze a reaction, the genes involved in each will be associated via an OR relationship. For reactions associated with multiple genes in this manner, the Boolean expression is evaluated by taking the sum or the mean of linear-scale expression values (e.g., TPMs) when genes are associated via an OR or AND relationship, respectively. This way, the full gene(s)-to-reaction associations are evaluated to arrive at a single, summary expression value for each reaction in the GEM.

The output of this procedure defines reaction expression as rᵢ^{(c)} for each reaction, i, and each cell, c. This defines the R matrix.

### Sharing Information Between Single Cells

To mitigate the sparseness and stochasticity inherent in single-cell measurements, Compass allows for a degree of information-sharing between cells with similar transcriptional profiles. To accomplish this, a neighborhood reaction expression is computed for each cell which represents a weighted average over expression measurements for similar cells in the data set. To compute this neighborhood reaction expression, two procedures are available to be selected at runtime: k-nearest neighbors (knn) or gaussian. Regardless of choice, first, the full gene expression matrix is reduced to a lower dimensional representation with PCA (20 components). Next, if the gaussian method is selected, a gaussian kernel is used to define cell-to-cell weights which describe the local neighborhood around each cell:

$${w}_{i j} = \frac{{Δ}_{i j}}{{σ}_{i}^{2}}$$

where Δᵢⱼ represents the Euclidean distance between cell i and cell j in the reduced PCA space and σᵢ² is computed for each cell using a supplied perplexity parameter and the method as described in the tSNE algorithm⁵⁵. The weights for each cell (rows of the w matrix) are then normalized to sum to 1. Alternately, if the knn method is selected, the weights w₁₁ are defined as 1/k if cell j is one of the k-nearest-neighbors (in the reduced PCA-space) of cell i, and zero otherwise. The number of neighbors (k) can be defined by the user at run-time, though Applicants recommend values in the range of 10-30. The weights resulting from either method are then used as mixing coefficients to arrive at neighborhood reaction expression values, rᵢ(C):

r̅ⱼ^{(c)}=Σⱼw_{cj}rᵢ^{(j)}

The r̅ᵢ^{(c)} values define the R^{N} matrix.

### Reaction Penalties

Let p(r) be a monotonically decreasing penalty function defined on [0, ∞). Applicants took p(r): =1/(1+r). The overall reaction penalty vector is a combination of the individual reaction penalties, p(rᵢ^{(c)}), and the neighborhood reaction penalties p(rᵢ^{(c)}), with the parameter 0≤λ≤1 used to define the mixing ratio.

\hat{p}ᵢ^{(c)}=(1−λ)·p(rᵢ^{(c)})+λ·p(rᵢ^{(c)})

The \hat{p}ᵢ^{(c)} values define the \hat{P} matrix.

### Compass Scores

The reaction penalties described up to this point only make use of the expression data associated with individual reactions. To integrate the full topology and stoichiometry of the GEM into the determination of the networks ability to carry flux through a reaction, Applicants make use of Flux Balance Analysis.

First, the GEM is transformed to be unidirectional. Each reaction is split into a pair of reactions proceeding in opposite directions and with added constraints only allowing positive reaction flux.

For simplicity of notation, Applicants define Compass below with the set of objective functions used in this application. Namely, m objectives where each one is maximization of one of the m unidirectional models in the network. Applicants further ignore the presence of blocked reactions, that in practice can be excluded to speed the computation. One may supplement or replace these objectives with other linear functions that pertain to cellular metabolism, such as maximization of biomass or ATP production.

Let S be the stoichiometric matrix defined in the GEM, where rows represent metabolites, columns represent reactions, and entries are stoichiometrical coefficients for the reactions comprising the metabolic network. Reactions for uptake and secretion of a metabolite are encoded as having only a coefficient of 1 and −1 in the metabolite's row entry, respectively, and 0 otherwise.

For each reaction r, a linear program computes the maximum amount of flux the network can produce, subject to steady-state and directionality constraints. rev(r) is the reverse unidirectional reaction of r, which has the same stoichiometry but proceeds in the opposite direction.

vᵣ^{opt}=max{v∈ [symbol] ^{m}}vᵣ

s.t. (i) S·v=0

(ii) α≤v≤β

(iii) vᵣₑᵥ₍ᵣ₎=0

Constraint (i) constrains the system to steady state (Varma and Palsson 1994), and constraint (ii) is interpreted as ∀i=1, . . . , n. αᵢ≤vᵢ≤βᵢ and encodes directionality and capacity limits for reactions, including uptake and secretion limits. Constraint (iii) ensures that when evaluating the maximum flux for each reaction, its reverse reaction carries flux to avoid the creation of a futile cycle. This does not prevent futile cycles longer than 2 edges, which can be avoided only by more time-consuming computations (Schellenberger et al. 2011).

Note that this computation described thus far is independent of any expression values and can be computed in advance for each GEM and cached for computational efficiency.

Next, for each reaction, a second linear program is used to evaluate the ability of the network to produce flux near this optimal value, while penalizing flux through reactions with lower expression support. This program minimizes the dot product between the flux distribution vector and reaction penalties while constraining the flux for the current reaction to remain within ω=0.95 range of its optimum. The minimum penalty yᵣ(c) for cell c and reaction r is:

yᵣ^{(c)}=min{v∈ [symbol] ^{m}}Σᵢvᵢ\hat{p}ᵢ^{(c)}

s.t. (i) S·v=0

(ii) α≤v≤β

(iii) vᵣₑᵥ₍ᵣ₎=0

(iv) vᵣ≥ω·vᵣ^{opt}

(recall that that the GEM is unidirectional and therefore ∀i. vᵢ>0)

A high penalty yᵣ(c) indicate that cell c is unlikely, judged by transcriptomic evidence, to use reaction r. Cells whose transcriptome are overall more aligned with an ability to carry flux through a reaction will be assigned a lower penalty for that reaction.

The minimum penalty yᵣ^{(c)} define the matrix C^{raw}, which has only non-negative entries by definition. Applicants transform it into a non-negative matrix where high score indicate high propensity to use a certain reaction by taking −log(1+C^{raw}) and then subtracting the minimal value of the resulting matrix from all its entries.

The resulting scores are indicative of a cell's propensity to use a certain reaction. Applicants interpret it as a proxy for the activity level of the reaction in that cell.

### Metabolite Scores

Applicants also implemented a second variant of the Compass procedure described above, where objective functions are based on the network's metabolites, rather than reactions. For every metabolite, Applicants define two objective functions—one to maximize its uptake, and one to maximize its secretion.

To assign uptake and secretion scores for a given metabolite, the procedure described above is used with a small modification. If an uptake or secretion reactions exist already in the GEM, they are evaluated in the same manner as other metabolic reactions and the resulting reaction score is used. Otherwise, an uptake/secretion reaction is added to the GEM and its resulting score is used.

### Example 7 References

1. Hotamisligil, G. S. Foundations of Immunometabolism and Implications for Metabolic Health and Disease. Immunity 47, 406-420 (2017).
2. O'Neill, L. A. J., Kishton, R. J. & Rathmell, J. A guide to immunometabolism for immunologists. Nat. Rev. Immunol. 16, 553-565 (2016).
3. Geltink, R. I. K., Kyle, R. L. & Pearce, E. L. Unraveling the Complex Interplay Between T Cell Metabolism and Function. Annu. Rev. Immunol. 36, 461-488 (2018).
4. Russell, D. G., Huang, L. & VanderVen, B. C. Immunometabolism at the interface between macrophages and pathogens. Nat. Rev. Immunol. (2019). doi:10.1038/s41577-019-0124-9
5. Wagner, A., Regev, A. & Yosef, N. Revealing the vectors of cellular identity with single-cell genomics. Nat. Biotechnol. 34, 1145-1160 (2016).
6. Orth, J. D., Thiele, I. & Palsson, B. O. What is flux balance analysis? Nat. Biotechnol. 28, 245-248 (2010).
7. O'Brien, E. J., Monk, J. M. & Palsson, B. O. Using Genome-scale Models to Predict Biological Capabilities. Cell 161, 971-987 (2015).
8. Lewis, N. E., Nagarajan, H. & Palsson, B. O. Constraining the metabolic genotype—phenotype relationship using a phylogeny of in silico methods. Nat. Rev. Microbiol. 10, 291-305 (2012).
9. Korn, T., Bettelli, E., Oukka, M. & Kuchroo, V. K. IL-17 and Th17 Cells. Annu. Rev. Immunol. 27, 485-517 (2009).
10. Tesmer, L. A., Lundy, S. K., Sarkar, S. & Fox, D. A. Th17 cells in human disease. Immunol. Rev. 223, 87-113 (2008).
11. Stockinger, B. & Omenetti, S. The dichotomous nature of T helper 17 cells. Nat. Rev. Immunol. 17, 535-544 (2017).
12. Wu, X., Tian, J. & Wang, S. Insight Into Non-Pathogenic Th17 Cells in Autoimmune Diseases. Front. Immunol. 9, 1112 (2018).
13. Wang, C. et al. CDSL/AIM Regulates Lipid Biosynthesis and Restrains Th17 Cell Pathogenicity. Cell 163, 1413-1427 (2015).
14. Berod, L. et al. De novo fatty acid synthesis controls the fate between regulatory T and T helper 17 cells. Nat. Med. 20, 1327-1333 (2014).
15. Michalek, R. D. et al. Cutting edge: distinct glycolytic and lipid oxidative metabolic programs are essential for effector and regulatory CD4+ T cell subsets. J. Immunol. 186, 3299-3303 (2011).
16. Shi, L. Z. et al. HIF1alpha-dependent glycolytic pathway orchestrates a metabolic checkpoint for the differentiation of TH17 and Treg cells. J. Exp. Med. 208, 1367-1376 (2011).
17. Araujo, L., Khim, P., Mkhikian, H., Mortales, C.-L. & Demetriou, M. Glycolysis and glutaminolysis cooperatively control T cell function by limiting metabolite supply to N-glycosylation. Elife 6, (2017).
18. Xu, T. et al. Metabolic control of TH17 and induced Tregcell balance by an epigenetic mechanism. Nature 548, 228-233 (2017).
19. Gaublomme, J. T. et al. Single-Cell Genomics Unveils Critical Regulators of Th17 Cell Pathogenicity. Cell 163, 1400-1412 (2015).
20. Dang, E. V. et al. Control of T(H)17/T(reg) balance by hypoxia-inducible factor 1. Cell 146, 772-784 (2011).
21. Rodriguez-Prados, J.-C. et al. Substrate fate in activated macrophages: a comparison between innate, classic, and alternative activation. J. Immunol. 185, 605-614 (2010).
22. Krawczyk, C. M. et al. Toll-like receptor-induced changes in glycolytic metabolism regulate dendritic cell activation. Blood 115, 4742-4749 (2010).
23. Donnelly, R. P. et al. mTORC1-dependent metabolic reprogramming is a prerequisite for NK cell effector function. J. Immunol. 193, 4477-4484 (2014).
24. Gubser, P. M. et al. Rapid effector function of memory CD8+ T cells requires an immediate-early glycolytic switch. Nat. Immunol. 14, 1064-1072 (2013).
25. Thiele, I. et al. A community-driven global reconstruction of human metabolism. Nat. Biotechnol. 31, 419-425 (2013).
26. Macosko, E. Z. et al. Highly Parallel Genome-wide Expression Profiling of Individual Cells Using Nanoliter Droplets. Cell 161, 1202-1214 (2015).
27. Klein, A. M. et al. Droplet Barcoding for Single-Cell Transcriptomics Applied to Embryonic Stem Cells. Cell 161, 1187-1201 (2015).
28. Hoppe, A. What mRNA Abundances Can Tell us about Metabolism. Metabolites 2, 614-631 (2012).
29. Nielsen, J. It Is All about Metabolic Fluxes. J. Bacteriol. 185, 7031-7035 (2003).
30. Khatri, P., Sirota, M. & Butte, A. J. Ten years of pathway analysis: current approaches and outstanding challenges. PLoS Comput. Biol. 8, e1002375 (2012).
31. Auslander, N., Wagner, A., Oberhardt, M. & Ruppin, E. Data-Driven Metabolic Pathway Compositions Enhance Cancer Survival Prediction. PLoS Comput. Biol. 12, e1005125 (2016).
32. Palsson, B. ø. Systems Biology: Constraint-based Reconstruction and Analysis (2nd ed.). (Cambridge University Press, 2015).
33. Becker, S. A. & Palsson, B. O. Context-Specific Metabolic Networks Are Consistent with Experiments. PLoS Comput. Biol. 4, e1000082 (2008).
34. Richelle, A., Joshi, C. & Lewis, N. E. Assessing key decisions for transcriptomic data integration in biochemical networks. bioRxiv 301945 (2018). doi:10.1101/301945
35. Vento-Tormo, R. et al. Single-cell reconstruction of the early maternal-fetal interface in humans. Nature 563, 347-353 (2018).
36. Wagner, F., Yan, Y. & Yanai, I. K-nearest neighbor smoothing for high-throughput single-cell RNA-Seq data. bioRxiv 217737 (2018). doi:10.1101/217737
37. Haghverdi, L., Lun, A. T. L., Morgan, M. D. & Marioni, J. C. Batch effects in single-cell RNA-sequencing data are corrected by matching mutual nearest neighbors. Nat. Biotechnol. 36, 421-427 (2018).
38. Ghoreschi, K. et al. Generation of pathogenic T(H)17 cells in the absence of TGF-β signaling. Nature 467, 967-971 (2010).
39. Lee, Y. et al. Induction and molecular signature of pathogenic TH17 cells. Nat. Immunol. 13, 991-999 (2012).
40. Yosef, N. et al. Dynamic regulatory network controlling TH17 cell differentiation. Nature 496, 461-468 (2013).
41. Romani, L. et al. Defective tryptophan catabolism underlies inflammation in mouse chronic granulomatous disease. Nature 451, 211-215 (2008).
42. Stephens, G. L. et al. Kynurenine 3-monooxygenase mediates inhibition of Th17 differentiation via catabolism of endogenous aryl hydrocarbon receptor ligands. Eur. J. Immunol. 43, 1727-1734 (2013).
43. Favre, D. et al. Tryptophan catabolism by indoleamine 2,3-dioxygenase 1 alters the balance of TH17 to regulatory T cells in HIV disease. Sci. Transl. Med. 2, 32ra36 (2010).
44. Hosios, A. M. et al. Amino Acids Rather than Glucose Account for the Majority of Cell Mass in Proliferating Mammalian Cells. Dev. Cell 36, 540-549 (2016).
45. Hui, S. et al. Glucose feeds the TCA cycle via circulating lactate. Nature 551, 115-118 (2017).
46. Bordbar, A. et al. Minimal metabolic pathway structure is consistent with associated biomolecular interactions. Mol. Syst. Biol. 10, (2014).
47. Damiani, C. et al. Integration of single-cell RNA-seq data into population models to characterize cancer metabolism. PLoS Comput. Biol. 15, e1006733 (2019).
48. Lewis, N. E. et al. Omic data from evolved E. coli are consistent with computed optimal growth from genome-scale models. Mol. Syst. Biol. 6, (2010).
49. Adler, M., Korem Kohanim, Y., Tendler, A., Mayo, A. & Alon, U. Continuum of Gene-Expression Profiles Provides Spatial Division of Labor within a Differentiated Cell Type. Cell Syst 8, 43-52.e5 (2019).
50. DeTomaso, D. et al. Functional Interpretation of Single-Cell Similarity Maps. bioRxiv 403055 (2018). doi:10.1101/403055
51. Fletcher, R. B. et al. Deconstructing Olfactory Stem Cell Trajectories at Single-Cell Resolution. Cell Stem Cell 20, 817-830. e8 (2017).
52. Cole, M. B. et al. Performance Assessment and Selection of Normalization Procedures for Single-Cell RNA-Seq. Cell Syst 8, 315-328.e8 (2019).
53. Ritchie, M. E. et al. limma powers differential expression analyses for RNA-sequencing and microarray studies. Nucleic Acids Res. 43, e47 (2015).
54. Law, C. W., Chen, Y., Shi, W. & Smyth, G. K. voom: Precision weights unlock linear model analysis tools for RNA-seq read counts. Genome Biol. 15, R29 (2014).
55. van der Maaten, L. Visualizing Data using t-SNE. J. Mach. Learn. Res. 9, 2579-2605 (2008).
56. Fefferman, C., Mitter, S. & Narayanan, H. Testing the manifold hypothesis. J. Amer. Math. Soc. 29, 983-1049 (2016).
57. Zhao, Q., Stettner, A. I., Reznik, E., Paschalidis, I. C. & Segrè, D. Mapping the landscape of metabolic goals of a cell. Genome Biol. 17, 1-11 (2016).
58. Gerriets, V. A. et al. Metabolic programming and PDHK1 control CD4+ T cell subsets and inflammation. J. Clin. Invest. 125, 194-207 (2015).
59. Campbell, D. J. & Butcher, E. C. Intestinal attraction: CCL25 functions in effector lymphocyte recruitment to the small intestine. J. Clin. Invest. 110, 1079-1081 (2002).
60. Trivedi, P. J. et al. Intestinal CCL25 expression is increased in colitis and correlates with inflammatory activity. J. Autoimmun. 68, 98-104 (2016).
61. Yoshie, 0. & Matsushima, K. CCR4 and its ligands: from bench to bedside. Int. Immunol. 27, 11-20 (2015).

### Example 8—Metabolic and Epigenomic Regulation of Th17/Treg Balance by the Polyamine Pathway

Cellular metabolism can orchestrate immune cell function. Applicants previously demonstrated that lipid biosynthesis represents one such gatekeeper to Th17 cell functional state. Utilizing Compass, a transcriptome-based algorithm for prediction of metabolic flux, Applicants constructed a comprehensive metabolic circuitry for Th17 cell function and identified the polyamine pathway as a candidate metabolic node, the flux of which regulates the inflammatory function of T cells. Testing this prediction, Applicants found that expression and activities of enzymes of the polyamine pathway were enhanced in pathogenic Th17 cells and suppressed in regulatory T cells. Perturbation of the polyamine pathway in Th17 cells suppressed canonical Th17 cell cytokines and promoted the expression of Foxp3, accompanied by dramatic shift in transcriptome and epigenome, transitioning Th17 cells into a Treg-like state. Genetic and chemical perturbation of the polyamine pathway resulted in attenuation of tissue inflammation in an autoimmune disease model of central nervous system, with changes in T cell effector phenotype.

### Introduction

Th17 cells and FoxP3+ regulatory T cells play a key role in maintaining the balance between inflammatory and regulatory functions in the immune system. One key aspect is the balance between Th17 and Treg cells. FoxP3⁺ Tregs play a critical role in maintaining immune tolerance, highlighted by loss-of-function mutations in the Foxp3 gene in human, the master regulator of Tregs, results in the development of IPEX syndrome where patients develop a series of autoimmune pathologies (autoimmune enteropathy, type 1 diabetes, dermatitis) and die prematurely. In contrast, Th17 cells have been shown to be critical for the induction of a number of autoimmune diseases including psoriasis, psoriatic arthritis, ankylosing spondylitis, multiple sclerosis and inflammatory bowel disease [1, 2]. While TGFβ alone can induce FoxP3⁺ Tregs in vitro, the addition of proinflammatory cytokine IL-6 suppresses the generation of FoxP3⁺ T cells and together with TGFβ induces generation of Th17 cells. This led to the hypothesis that proinflammatory Th17 and regulatory FoxP3⁺ Tregs are reciprocally regulated, further supported by experiments on the role of these two cytokines in the induction and differentiation of Th17 cells in vivo [3-6].

However, not all Th17 cells are pathogenic or disease inducing, and they also play a protective role in mucosal tissues, promoting tissue homeostasis, maintaining barrier function as well as preventing invasion of microbiota at the mucosal sites [7-12]. Th17 cells that are induced by TGFb+IL-6 in vitro, produce IL-17 but are not capable of inducing potent tissue inflammation/autoimmunity upon adoptive transfer [13-15]. Additional stimuli, such as IL-1b and IL-23, are needed to evoke pathogenic potential in these Th17 cells [13, 14, 16-20]. Therefore, there appear to be at least two different types of Th17 cells: Th17 cells that are present at homeostasis and do not promote tissue inflammation that Applicants have termed nonpathogenic Th17 cells and the Th17 cells which produce IL-17 together with IFN-g and GMCSF induce tissue inflammation and autoimmunity [21]. Different types of Th17 cells have also been identified in humans where Th17 cells akin to mouse pathogenic Th17 cells have been shown to be specific for Candida albicans and non-pathogenic Th17 cells have been shown to be similar to Th17 cells that have specificity for Staphylococcus aureus infection [22]. Thus, Treg, non-pathogenic Th17 cells and pathogenic Th17 cells represent a functional spectrum in tissue homeostasis, disease and infection and can be differentiated reciprocally with different cytokine cocktails in vitro. However, in addition to cytokines, how these cells are generated in vivo and what are the factors that trigger their development of different functional states has not been fully elucidated.

Cellular metabolism is a mediator and modulator of immune cell differentiation and function, which Applicants hypothesized may play a key role in this balance. In a previous study using scRNA-seq of Th17 cells, Applicants identified CDSL as a major regulator that co-varies in its expression with the pro-inflammatory gene module in Th17 cells. Loss of CDSL made Th17 cells highly pathogenic by altering lipid biosynthesis and transcriptional activity of RoR γt, the master transcription factor critical for development and differentiation of Th17 cells [23]. This observation provided a proof of concept that metabolic processes can be directly involved in gene regulation and balancing proinflammatory and regulatory states of Th17 cells.

However, a full appreciation of metabolic circuitry and its connection with immune cell function has been limited by available technologies that typically define the average metabolic state of a large population of cells. Applicants have developed a flux balance analysis algorithm called Compass that allows prediction of metabolic state of a cell using transcriptome data at the single cell level, allowing comprehensive profiling of metabolic pathways even in a smaller number of cells that could not be otherwise interrogated by traditional metabolomic techniques (Wagner et al, 2020). Here, Applicants used the Compass algorithm to interrogate the metabolic status of pathogenic and nonpathogenic Th17 cells using scRNA-seq datasets of Th17 cells. Applicants show that enzymes of the polyamine pathway are suppressed and cellular polyamine content is significantly lower in regulatory T cells and non-pathogenic Th17 cells (Th17n) as compared to pathogenic Th17 cells (Th17p) due to alternative fluxing. Perturbation of the polyamine pathway in Th17 cells suppressed canonical Th17 cytokines and promoted Foxp3 expression, shifting the Th17 cell transcriptome in favor of a Treg-like state. Applicants demonstrated that the polyamine pathway is critical in maintaining the Th17-specific chromatin landscape against the induction of Tregs-like program. Consistent with the cellular phenotype, chemical inhibition and genetic perturbation of the polyamine pathway in T cells restricted the development of autoimmune responses in the EAE model.

### Identifying the Polyamine Pathway as a Candidate in Regulating Th17 Cell Function

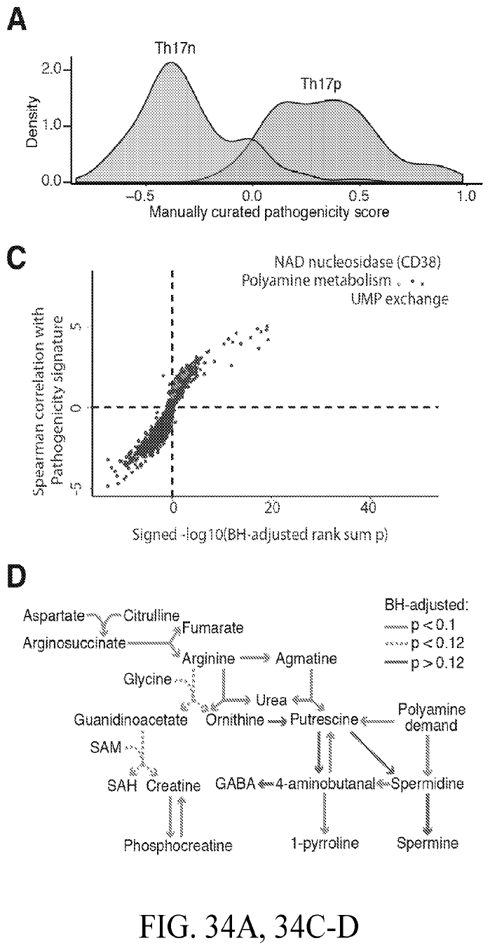

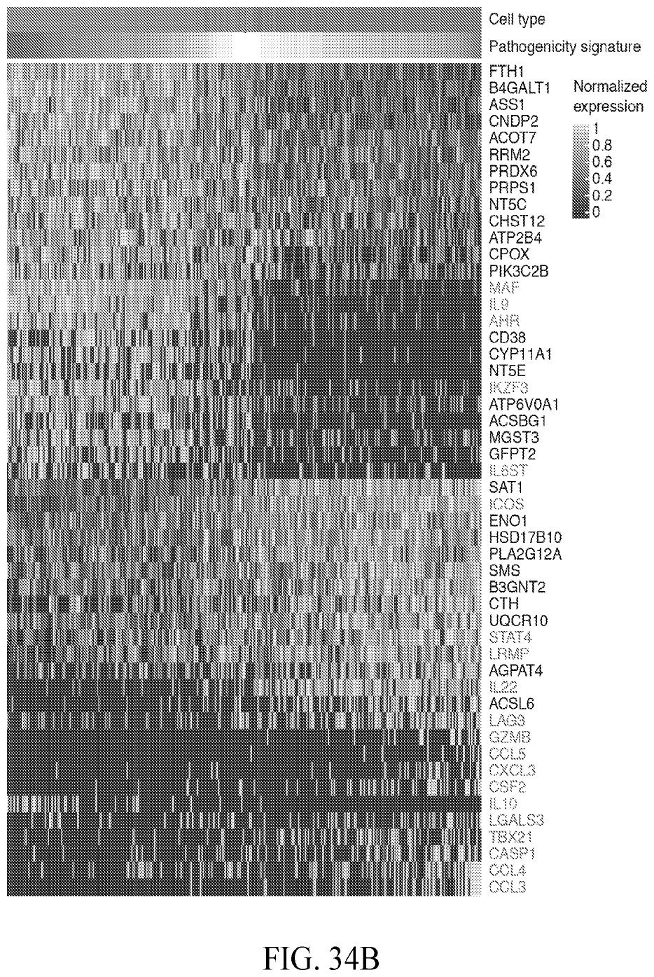

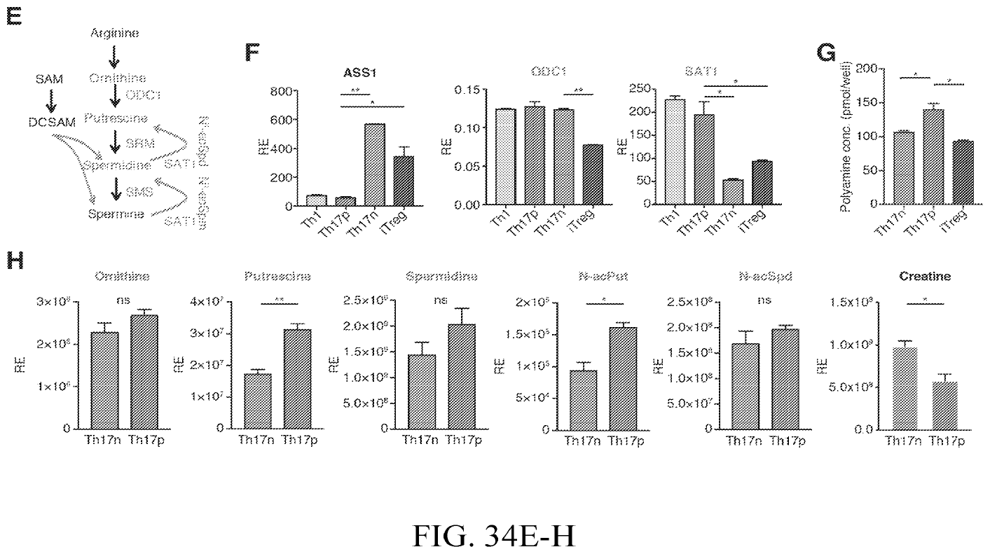

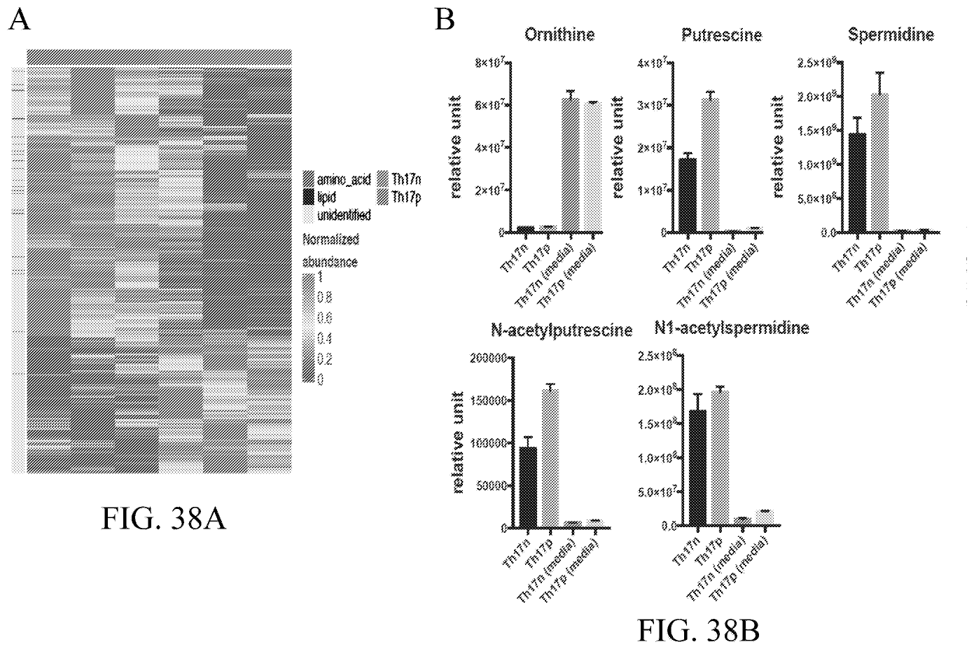

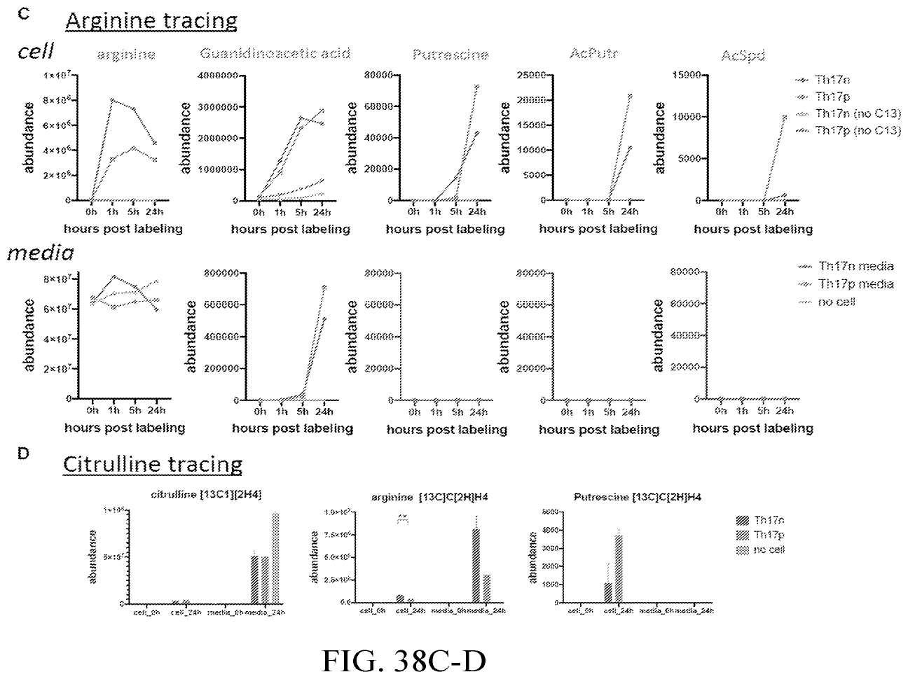

To better analyze the metabolic landscape of Th17 cells that may regulate their functional state, Applicants first used two approaches: untargeted metabolomics (FIG. 38) and standard analysis of single-cell RNAseq data (FIG. 34A, B). For both analyses, Applicants compared Th17 cells differentiated from naïve CD4+ T cells using two combinations of cytokines: IL-1b+IL-6+IL-23 (Th17p, pathogenic) and TGFb+IL-6 (Th17n, non-pathogenic) that Applicants previously reported to either promote or restrict Th17 cell pathogenicity respectively in the context of the EAE model, and therefore represents the two extremes of functional state of Th17 cells [14, 23]. Untargeted metabolomics identified 1,101 (out of 7,436) metabolic features to be differentially expressed between Th17n and Th17p (BH-adjusted Welch t-test p<0.05; FIG. 38). Applicants identified 52 of the differentially expressed metabolites, a third of which (19/52) are of lipid nature, consistent with the previous finding that lipid biosynthesis is a key regulator of Th17 cell functions [23], and the rest related to multiple amino-acid pathways. Next, Applicants evaluated the expression of metabolic enzyme genes (“metabolic transcriptome”) of sorted IL-17-GFP+Th17 cells differentiated in vitro, which Applicants previously profiled by scRNA-seq [24]. The distributions of the computational pathogenicity signature scores (computed by expression of key cytokines and transcription factors [14, 24]) of cells from the two conditions were readily distinguishable (p<3*10⁻¹⁶, two-sided Welch t-test). However, there was considerable variation across the individual cells within each condition [23, 24], such that some of the cells from the Th17n condition has higher pathogenicity scores than cells from the Th17p condition (FIG. 34A), with a minor distinctive mode of more pathogenic-like cells. This intra-population heterogeneity highlights the benefit of studying the Th17n and Th17p populations at a single-cell level. Interestingly, many of the genes that most covaried with the genes associated with Th17 cell function belonged to ancillary metabolic pathways (FIG. 34B), as were most of the identified differentially expressed metabolites, rather than the central and well-studied glycolysis pathway.

Next, to obtain a comprehensive view of the metabolic state of each cell despite the inability to measure single cell metabolomic profiles, Applicants investigated the metabolic circuitry of Th17 cells using Compass (see example 9, STAR Methods), with the scRNA-seq profiles from sorted IL-17-GFP+Th17 cells [24]. Briefly, Compass is a Flux Balance Analysis (FBA)-based algorithm [25, 26] and utilizes a comprehensive compendium of thousands of metabolic reactions, their stoichiometry, and the enzymes catalyzing them [27]. Compass models in silico the fluxes through the network of metabolic reactions, while accounting for the observed expression levels of enzyme-coding transcripts in each cell. It does so by optimizing a series of objective functions, each corresponding to an individual metabolic reaction (rather than a single FBA objective such as biomass production). The result of the optimization procedure is a score for each reaction in each cell, indicative of the potential of the cell to direct flux through that reaction, given the transcriptome of that cell. The Compass scores matrix is then subject to downstream analysis, while relying on the statistical power afforded by scRNA-Seq to derive biological insight from the high-dimensional matrix.

Analysis of the Compass scores for each reaction across all single cells in the data (FIG. 34C) showed that among those metabolic reactions significantly correlated with Th17 cell pathogenicity, the polyamine pathway stood out as one that is differentially activated in pathogenic vs. nonpathogenic Th17 cells (FIG. 34C and Wang et al., 2020, Table S1). To explore this, Applicants constructed a data-driven metabolic network anchored around putrescine, the entrance metabolite into the canonical polyamine synthesis, by including adjacent metabolites whose reactions are predicted to be negatively associated with pathogenicity (FIG. 34D). While several polyamine-associated genes (e.g., Sat1 in FIG. 34B) are differentially expressed between Th17p and Th17n, the network tied the differential polyamine metabolism to differences in upstream and downstream metabolic reactions which could not be captured from differential gene expression directly. Specifically, Compass predicted that Th17n cells are more active in arginine metabolic pathways, lying upstream of putrescine, and in alternative fates of putrescine (other than conversion to spermidine, along the canonical polyamine synthesis pathway) (FIG. 34D). Applicants hypothesized that the arginine/polyamine pathway may be a metabolic bifurcation point that can regulate Th17 cell function and set out to investigate this metabolic network surrounding polyamines.

### Cellular Polyamines are Suppressed in Regulatory T Cells and Nonpathogenic Th17

To investigate the polyamine metabolic process (FIG. 34E), Applicants first asked whether critical enzymes of this pathway are differentially expressed in different CD4+ T cell subsets using qPCR. Ornithine Decarboxylase 1 (ODC1) and Spermidine/Spermine N1 Acetyltransferase 1 (SAT1) are the rate-limiting enzymes of polyamine biosynthesis and catabolic processes, respectively, and Ornithine decarboxylase antizyme 1 (OAZ1) can regulate the enzymatic activity of ODC1. ODC1 catalyzes ornithine to putrescine, the first step of the polyamines biosynthesis; SAT1 regulates the intracellular recycling of polyamines and their transport out of the cell. SAT1, but not ODC1 or OAZ1, was suppressed in Th17n vs. Th17p cells (FIG. 34F and data not shown). Intriguingly, both ODC1 and SAT1 expression was lower in Tregs, whereas Ass1, an enzyme upstream of the polyamine biosynthesis pathway is upregulated, consistent with Compass-predicted alternative flux in the polyamine neighborhood (FIG. 34F). Collectively, these data suggest the polyamine pathway may be associated with functional state beyond Th17 cells.

As the polyamine pathway, similar to most metabolic pathways, is regulated beyond the transcriptional level, Applicants next directly measured total cellular polyamine content using an enzymatic assay (STAR Methods). Compared to Th17p cells, Tregs and Th17n cells have significantly reduced levels of total polyamines (FIG. 34G), reflective of either reduced import, biosynthesis or increased export of polyamines in these cells.

To further investigate the concentrations and activities of different polyamines in Th17 cells at different functional states, Applicants applied both targeted metabolomics and carbon tracing. Applicants differentiated Th17n and Th17p cells for 68 hours (STAR Methods) and measured the amount of polyamines and related precursors in cell and media by LC/MS (FIGS. 34H and 38B). Consistent with Compass's predictions, there was higher creatine content in Th17n vs. Th17p cells. On the other hand, while the total amount of cellular ornithine, precursor to polyamines, was comparable between Th17n and Th17p cells, there was a significant increase of putrescine and acetyl-putrescine content in Th17p cells (FIG. 34H), indicative of increased activity of this pathway in Th17p cells, consistent with the enzymatic assay. Of note, cellular spermidine (or acetyl-spermidine) content was not different between the conditions, and spermine was not detected (FIG. 34H). The reduced putrescine and its acetyl form in Th17n cells are not due to increased export, as Applicants observed very little polyamines in the media in either Th17n or Th17p cells (FIG. 38B). These data suggest that polyamines accumulate within Th17p cells and the main function of SAT1 in Th17p cells may be to recycle rather than to export polyamines.

To directly investigate polyamine biosynthesis, Applicants cultured differentiated Th17n and Th17p cells in the presence of low amount of carbon or hydrogen labeled arginine or citrulline, which can be used to synthesize ornithine, precursor to the polyamine pathway (FIG. 38C, D). First, Applicants harvested cells and media for LC/MS at 0, 1, 5 and 24 hours post addition of arginine. While there was comparable accumulation of labeled cellular guanidinoacetic acid, a byproduct of arginine conversion into ornithine, in Th17n and Th17p cells over time (FIG. 38C), Th17p cells accumulated higher intracellular amounts of putrescine, acetylputrescine and acetylspermidine, consistent with increased polyamine biosynthesis and/or recycling activity in these cells (FIG. 38C). Conversely, there were higher levels of labeled arginine in Th17n cells vs. Th17p cells, prompting Applicants to investigate whether Th17n cells can better synthesize (as opposed to better uptake) arginine, which would be consistent with increased ASS1 expression (FIG. 34F) in these cells. To this end, Applicants harvested cells for LC/MS 24 hours after addition of labeled citrulline, a precursor to arginine synthesis. Indeed, there was higher accumulation of labeled arginine in Th17n cells (FIG. 38D). Collectively, the targeted metabolomics and carbon tracing data suggest that Th17n cells accumulate arginine, consistent with Compass's prediction (FIG. 34D), and that Th17p cells preferentially synthesize or recycle polyamines. Applicants conclude that differences in the alternative flux hinged on polyamine biosynthesis is associated with the different functional states of Th17 cells.

### ODC1 or SAT1 Inhibition Restricts Th17 Cell Function in a Putrescine-Dependent Manner

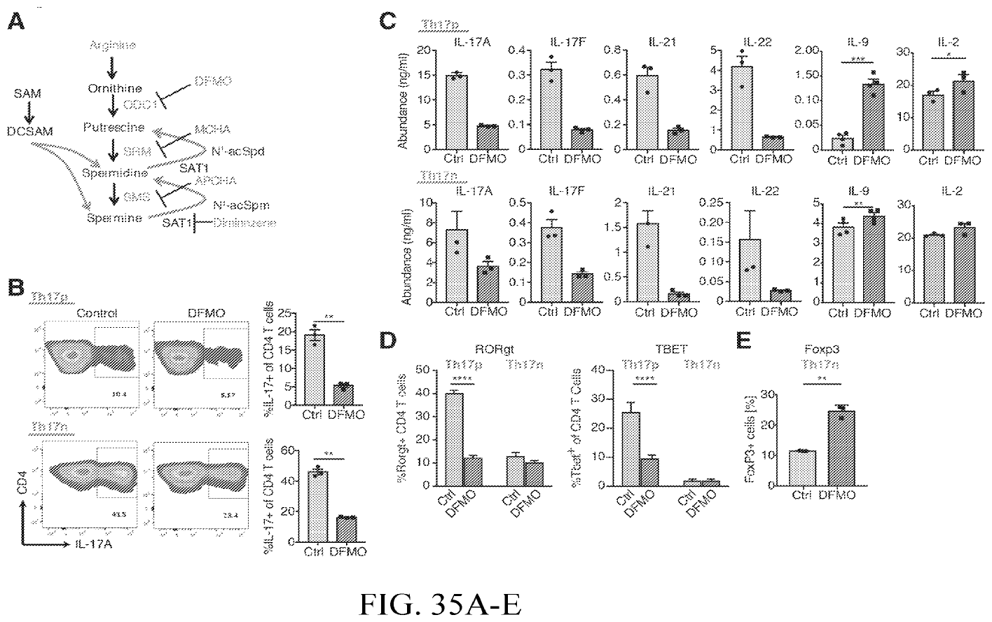

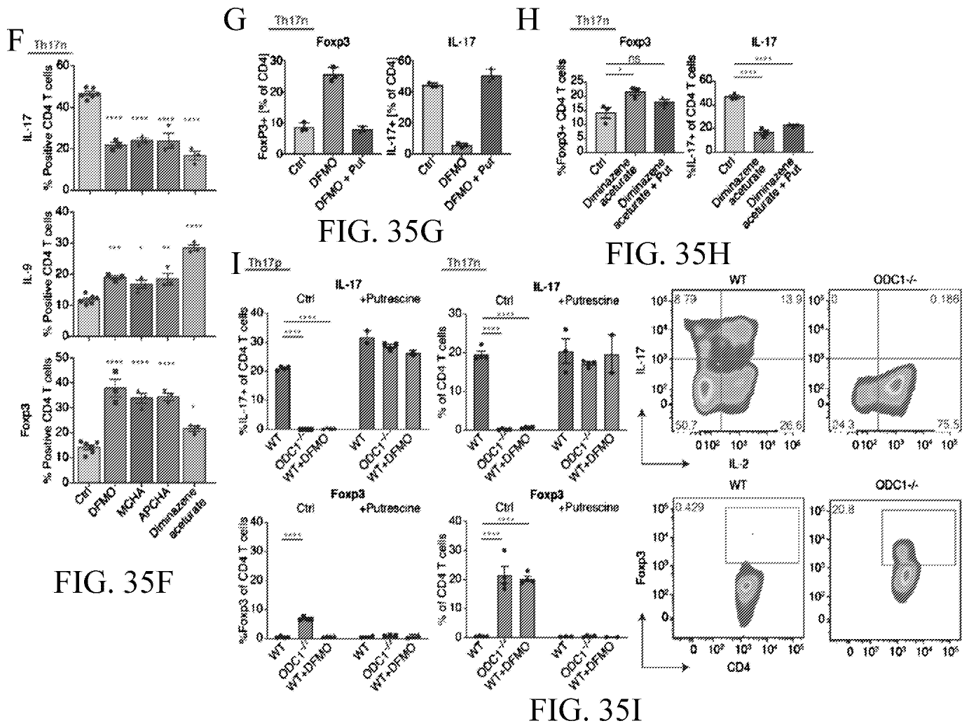

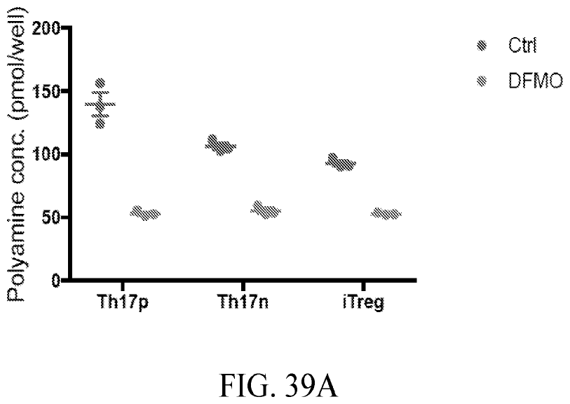

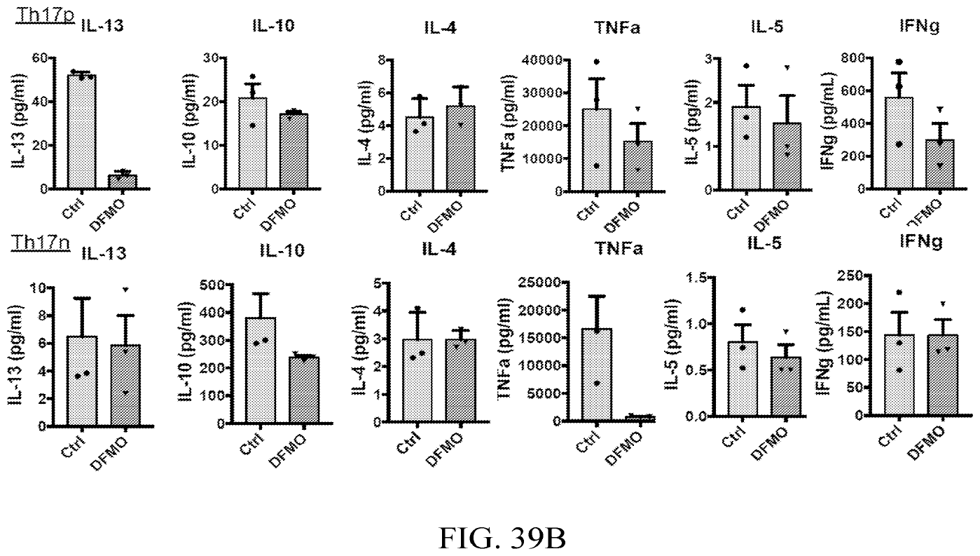

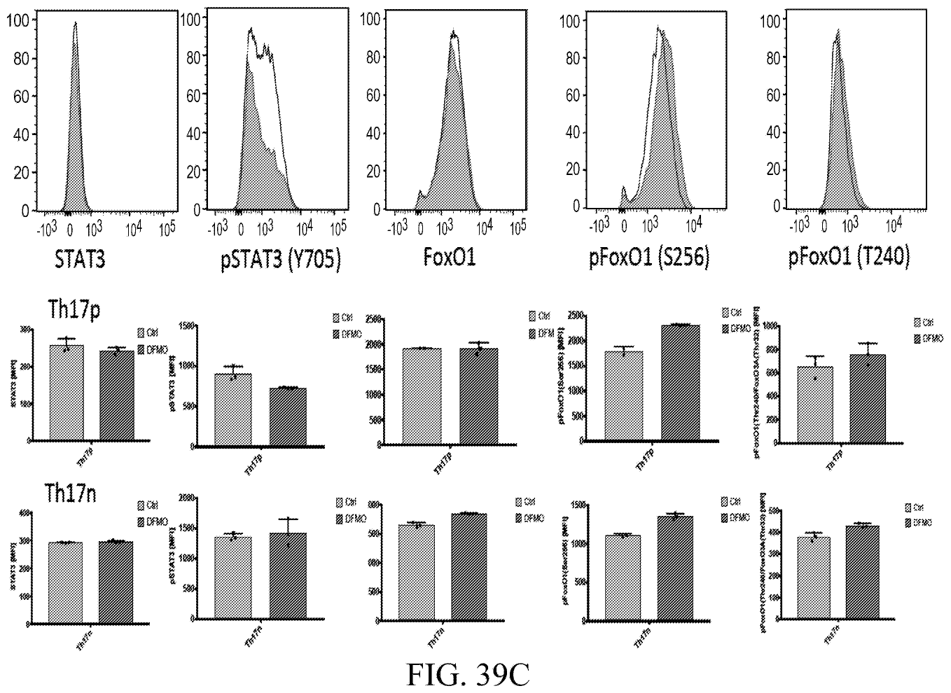

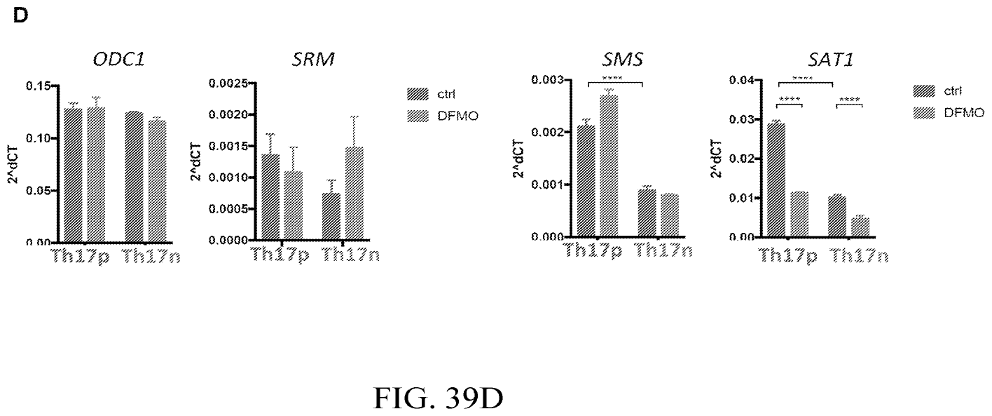

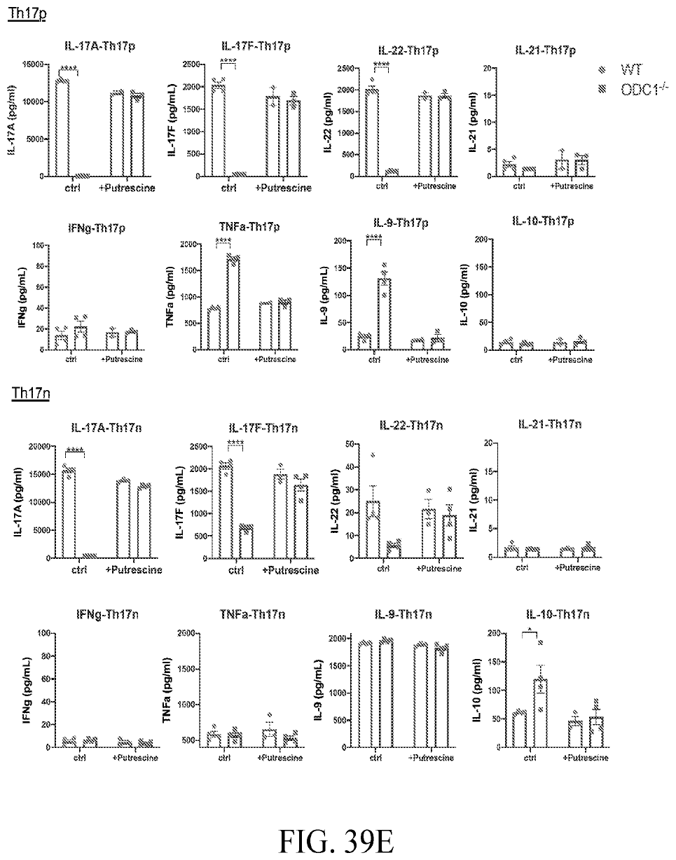

To investigate the functional relevance of these metabolic changes, Applicants studied the effects of polyamine pathway inhibitors on differentiation of pathogenic and nonpathogenic Th17 cells in vitro, using previously defined culture conditions. Applicants first used difluoromethylornithine (DFMO), an irreversible inhibitor of ODC1 (FIG. 35A), the enzyme that catalyzes the conversion of ornithine to putrescine. Enzymatic assays of in vitro differentiated Th17n, Th17p, or iTreg cells treated by DFMO confirmed its suppression of polyamines in all three cell types (FIG. 39A). At an optimized concentration where Applicants observed similar viability between control and treatment, DFMO significantly inhibited IL-17 expression in both Th17n and Th17p cells by intracellular staining and flow cytometry (FIG. 35B), as well other canonical Th17 cytokines such as IL-17A, IL-17F, IL-21 and IL-22, while promoting IL-9 expression in supernatant from both Th17n and Th17p cultures (FIG. 35C). DFMO did not consistently influence, IFNg, TNFa, IL-13, IL-10 or IL-5 expression (FIG. 39B). IL-17 inhibition does not appear to be solely related to regulation of IL-2 production [28], as DFMO promoted IL-2 expression in supernatant from only Th17p, but not Th17n cells (FIG. 35C). Polyamines can influence cell proliferation. While Applicants did observe reduced cell proliferation in cultures treated with DFMO, the frequency of IL-17+ cells was significantly reduced in cells that have divided just once (data not shown), suggesting DFMO can regulate Th17 cell function independent of cellular proliferation. The increase in IL-9 following DFMO treatment also supports the hypothesis that DFMO is not universally inhibiting viability of Th17 cells and enhances Th9 derived cytokines.

To determine whether DFMO inhibited Th17 cell differentiation, Applicants measured the expression and activity of key transcription factors. Interestingly, DFMO suppressed Rorgt and Tbet expression in Th17p but not Th17n cells (FIG. 35D), suggesting a nuanced effect. Consistently, DFMO decreased pStat3, and not total Stat3 protein levels, only in Th17p but not Th17n cells (FIG. 39C). IL-17 inhibition is not due to increased Foxo1 activity, another critical regulator of Th17 cell function, as DFMO promoted pFoxo1(S256) in both types of Th17 cells, which would have resulted in a net increase in IL-17 expression (FIG. 39C). Given the known reciprocal relationship between Th17 cells and Tregs, and as DMFO also impacted polyamine concentration in Tregs, Applicants asked whether DFMO can regulate Foxp3 expression in Th17 cells, even under Th17 differentiation conditions. Applicants observed increased frequency of Foxp3+ cells in Th17n but not Th17p conditions (FIG. 35E), presumably because TGFb is required for the differentiation in this condition and DFMO strengthens TGFb derived activity to induce Treg differentiation over Th17 cells.

To determine whether other enzymes of the polyamine pathway could play a similar role in regulating Th17 cell function, Applicants used inhibitors of spermine synthase (SRM), spermidine synthase (SMS), and SAT1 (FIG. 35A). Similar to DFMO, inhibitors of any of the polyamine biosynthesis enzymes resulted in suppression of IL-17 and upregulation of IL-9 and Foxp3 expression, the latter in Th17n cells (FIG. 35F). Furthermore, inhibiting SAT1 by diminazene, a rate-limiting enzyme of polyamine acetylation and recycling, had similar effects to DFMO (FIG. 35F). SAT1 perturbation was previously reported to have a feedback effect on ODC1 activity and vice versa [29-31]. Consistent with this finding, inhibition with DFMO consistently suppressed SAT1 expression in both Th17n and Th17p cells (FIG. 39D). Thus, it may be the flux of polyamines and not metabolites per se that modulate Th17 cell function.

Finally, Applicants confirmed that the effect of DFMO is through the inhibition of ODC1, as addition of putrescine to cells treated with DFMO completely reversed their phenotype (FIG. 35G). Interestingly, addition of putrescine during SAT1 inhibitor treatment also partially reversed the upregulation of Foxp3, but not suppression of IL-17 (FIG. 35H), suggesting putrescine flux may be particularly important in the control of the regulatory program in Th17 cells. Overall, the inhibitor data are consistent with a role of the polyamine pathway in regulating Th17 cell differentiation, but genome-wide profiling would be necessarily to further support this claim.

### ODC1^{-/-} Th17 Cells Promoted Foxp3 Expression

To further confirm the effects of chemical inhibition of polyamine pathway on Th17/Treg differentiation, Applicants tested the impact of genetic perturbation of ODC1 on the differentiation and functions of Th17 cells, using cells isolated from WT and ODC1−/− mice. Similar to DFMO treatment, there was complete inhibition of Th17 canonical cytokines, such as IL-17A, IL-17F and IL-22, but not IFNg, in ODC1−/− Th17 cells (FIG. 35I upper panel and 39E). ODC1 deficiency did not lead to a decrease in Rorgt expression (data not shown), but there was a dramatic loss of Th17 canonical cytokines, consistent with loss of the Th17 program. Furthermore, ODC1−/− Th17n cells upregulated Foxp3 expression, consistent with promotion of a Treg program (FIG. 35I, lower panel). Finally, all the observed effects of ODC1−/− were rescued by addition of putrescine (FIGS. 351 and 39E).

### DFMO Restricts Th17-Cell Transcriptome and Epigenome in Favor of Treg-Like State

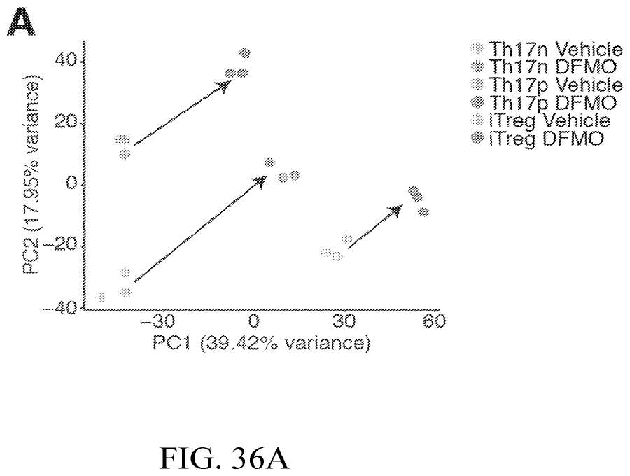

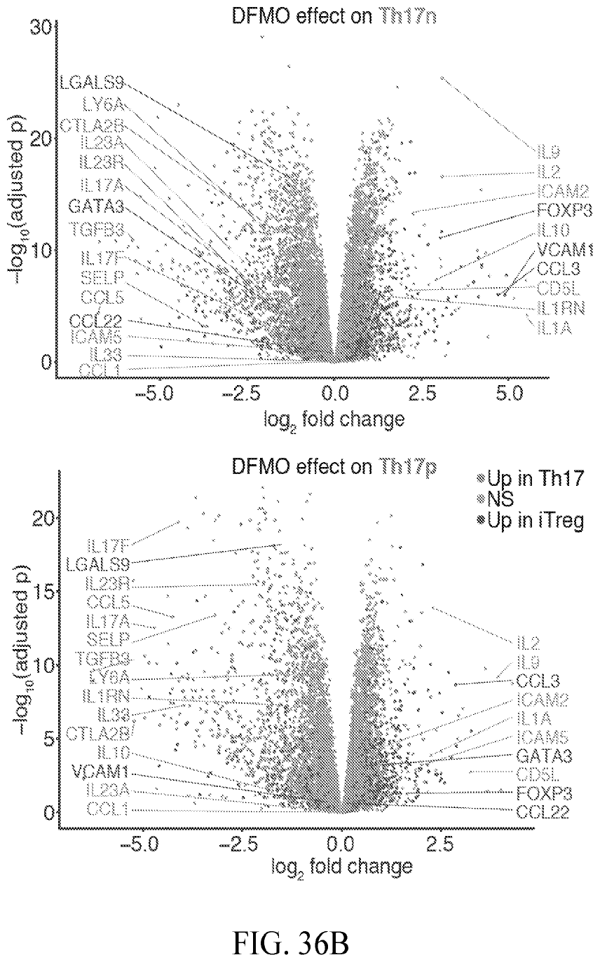

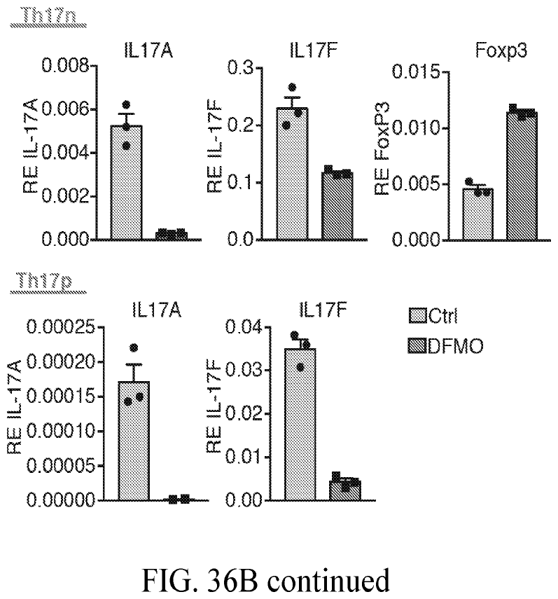

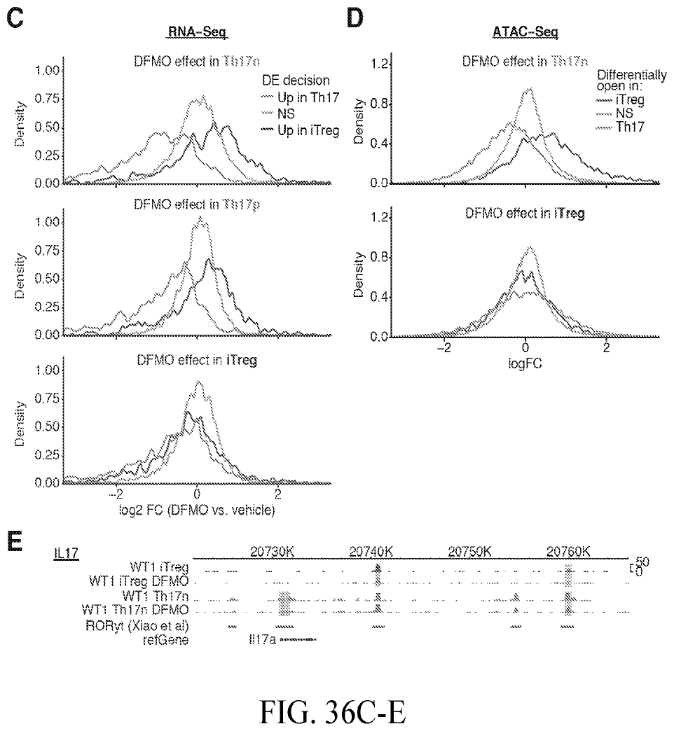

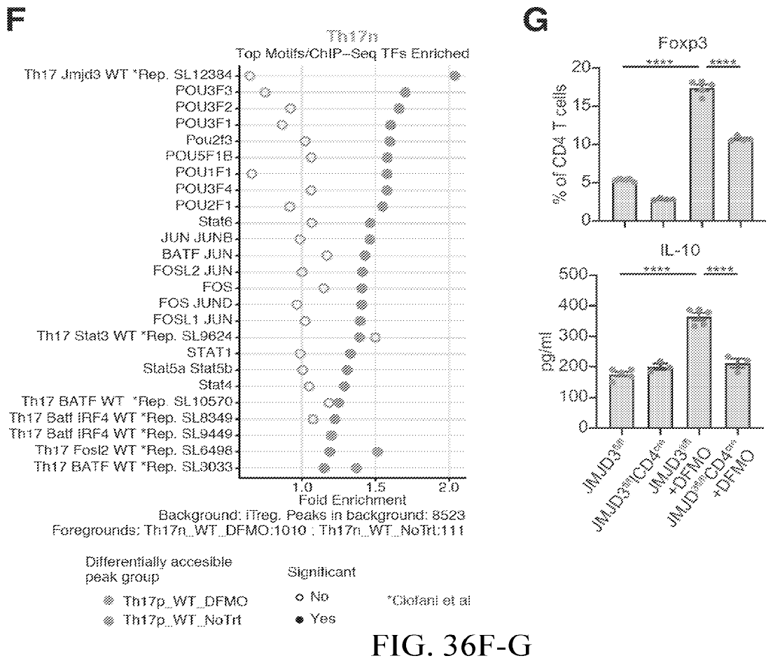

To gain mechanistic insight on the effects of inhibiting polyamine biosynthesis in Th17 cells, Applicants profiled by RNA-Seq Th17n, Th17p, and iTreg cells treated with DFMO or control. DFMO had a profound impact on the transcriptome of all Th cell lineages, driving Th17 cells towards Treg cell profiles in Principal Components Analysis (PCA) (FIG. 36A, PC1). To gain further insights, Applicants determined the aggregate effect of DFMO on genes up-regulated (n=1,284), down-regulated (n=1,255) or comparable (n=8,257) in Th17 vs. T_{reg} cells (FIG. 36B). In both Th17n and Th17p cells, DFMO suppressed the Th17 cell specific gene set, and promoted the Treg-specific transcriptome (FIG. 36C, Wang et al., 2020 Table S2 and S3). Specifically, canonical Th17 cell genes such as Il17a, Il17f and Il23r were significantly suppressed, whereas Treg related genes, such as Foxp3, were upregulated (FIG. 36B). There was no significant effect of DFMO treatment on genes expressed comparably in Th17 cells and Treg, nor did DFMO have an effect in Treg cells (FIGS. 36B and 36C). These results are consistent with a model where the polyamine pathway is important for restricting the iTreg-like program in Th17 cells at both functional states (FIG. 36A).

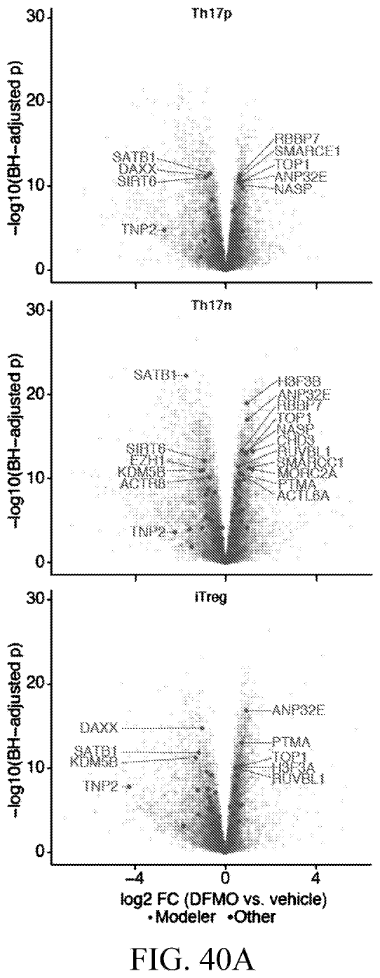

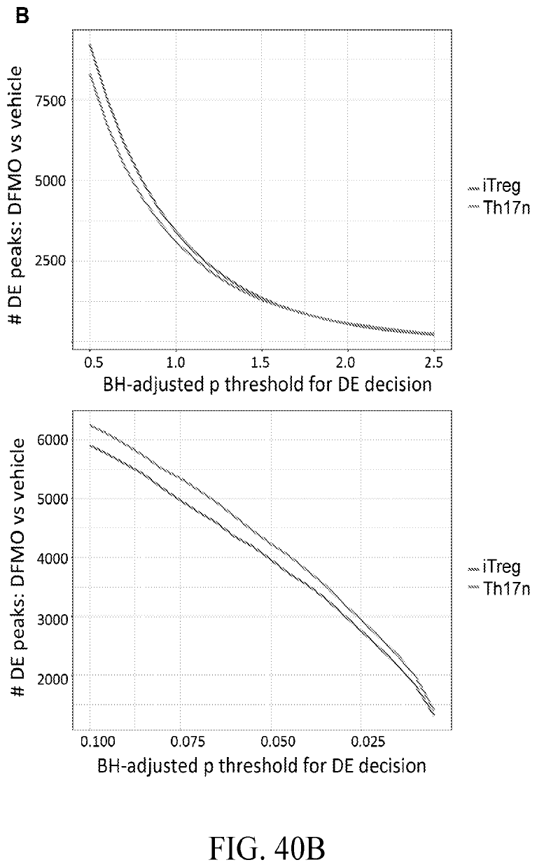

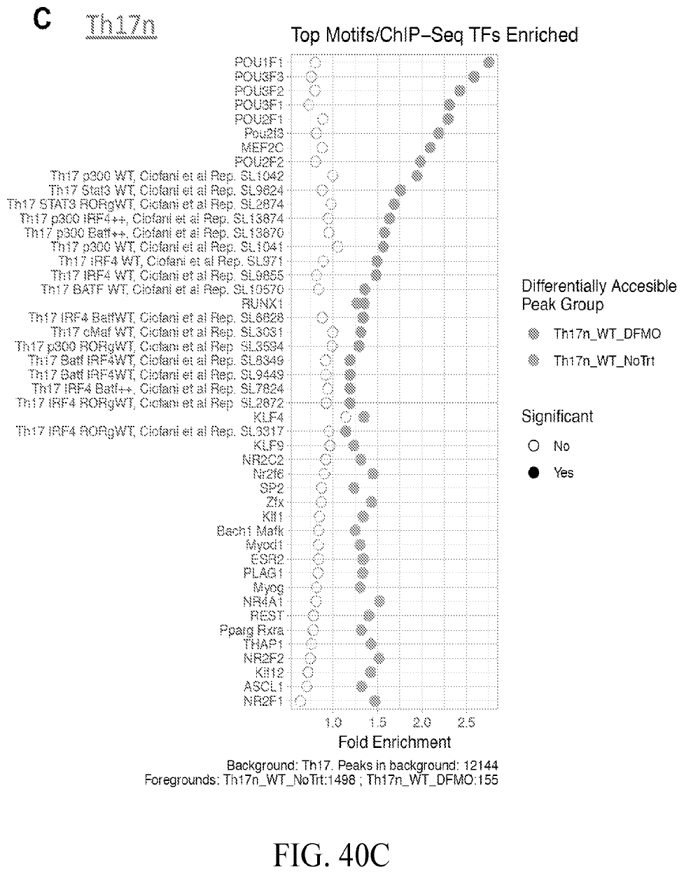

The profound impact of DFMO on the transcriptome prompted Applicants to investigate the mechanism by which the polyamine pathway regulates Th17 cell functions. As DFMO does not appear to consistently restrict phosphorylation of key Th17 cell regulators, particularly not in Th17n cells (FIG. 39C), Applicants hypothesized that polyamines may impact the epigenome. Consistent with a role of the polyamine pathway in affecting chromatin modification, Applicants observed significant changes in expression of many chromatin modifiers (FIG. 40A).

To test this hypothesis, Applicants measured chromatin accessibility by ATAC-seq in Th17n and iTregs cells treated with either control or DFMO (STAR Methods). Overall, DFMO treatment resulted in considerable changes in accessible peaks in both types of Th cells (FIG. 40B and Wang et al., 2020 Table S4A and S4B). Next, Applicants asked whether DFMO preferentially altered accessibility to regions specific to Th17 cells and iTregs. To this end, Applicants partitioned all accessible peaks into (1) those more accessible in Th17 cells (n=10,431), (2) more accessible in iTregs (n=3,421), and (3) comparably accessible in both (n=34,591) (FIG. 36D, Wang et al., 2020 Table S3, and STAR Methods). Consistent with the expression changes, following DFMO treatment there was a significant shift towards less accessibility in Th17 specific regions and more accessibility in Treg specific regions (FIG. 36D and Wang et al., 2020 Table S3). Differentially accessible regions were found near loci encoding key effector molecules (Wang et al., 2020 Table S4A and S4B). For instance, DFMO treatment significantly restricted peaks in the promoter and intergenic regions of Il17a-Il17f that corresponds to Rorgt binding site (using ChIP-seq data from [32]) known to regulate IL17 expression (FIG. 36E). Thus, DFMO treatment can shape chromatin accessibility in favor of an iTreg epigenomic landscape, and this may contribute to the emergence of iTreg transcriptional program in DFMO-treated Th17 cells.

### The Chromatin Regulator JMJD3 Partially Mediates the Polyamine-Dependent Effect on Th17 Cells

To investigate which transcription factors (TFs) may be responsible for the suppression of the Th17 specific program and upregulation of the iTreg program, Applicants looked for putative binding sites (based on DNA binding motifs or ChIP-seq data when available) that significantly overlap with regions whose accessibility is modulated by DFMO (FIG. 36F and Wang et al., 2020 Table S5). Applicants restricted the analysis to genomic regions that are typically accessible only in Tregs (compared to Th17 cells) and may be modulated by DFMO (FIGS. 36F and 40C). In Th17n cells, DFMO increased accessibility near potential binding sites of the chromatin regulator JMJD3 along with a number of POU-domain containing TFs.

As JMJD3 is a known regulator of T cell plasticity [33], Applicants tested whether it also contributes to the genome-wide shifts induced by DFMO. Applicants analyzed the effect of DFMO on Th17 cells differentiated from naïve CD4 T cells isolated from control or JMJD3fl/flCD4cre mice (FIG. 3G). Supporting the hypothesis, the upregulation of Foxp3 by DFMO in Th17n cells was partially abrogated in the absence of JMJD3, and loss of JMJD3 also reduced the DFMO-dependent upregulation of IL-10 in Th17n cells (FIG. 36G).

### Perturbation of ODC1 and SAT1, Key Enzymes of the Polyamine Pathway, Alleviates EAE

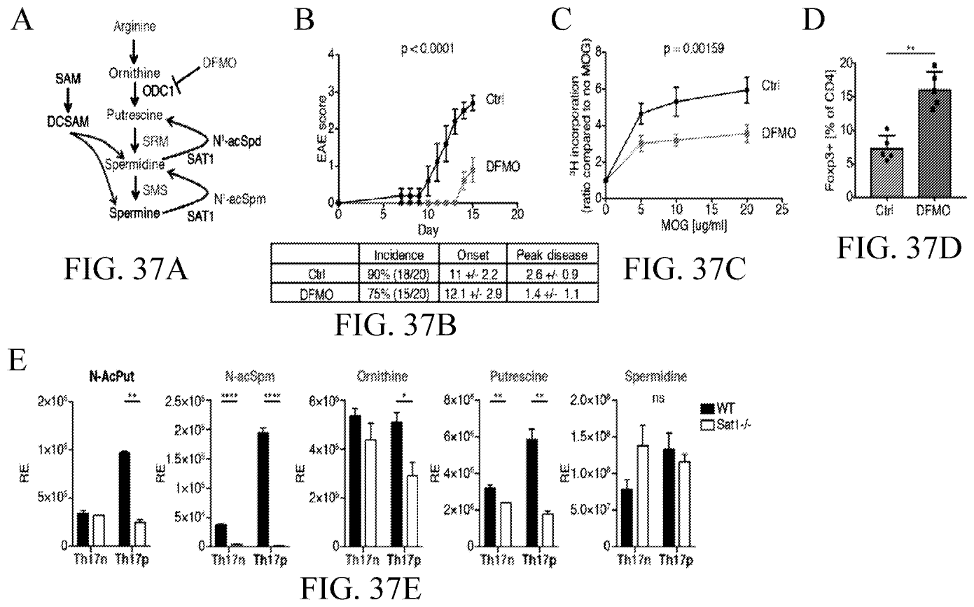

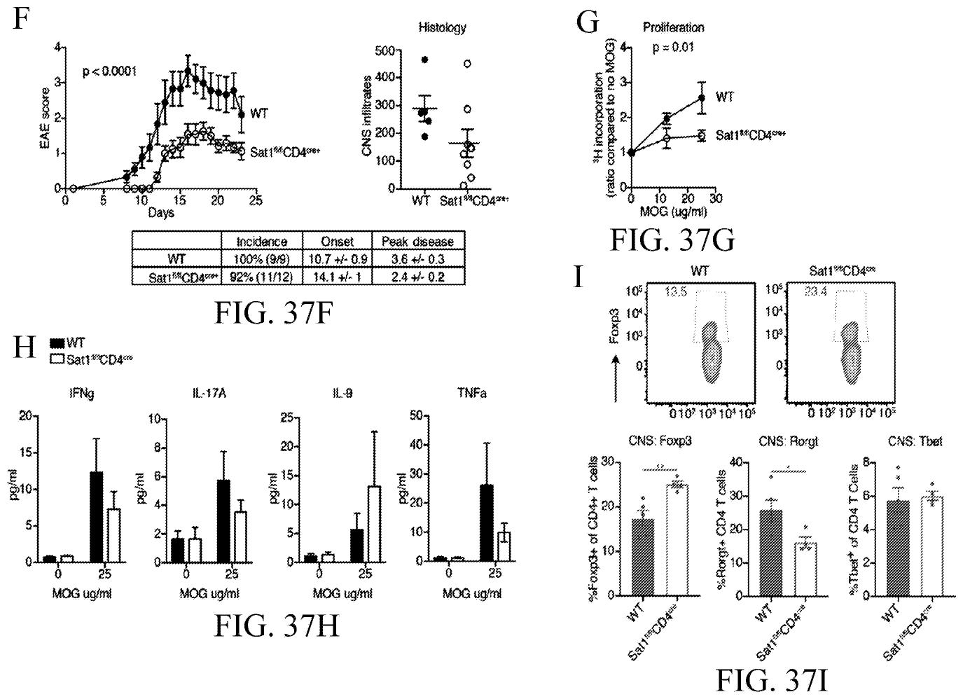

To investigate the relevance of the polyamine pathway in vivo, Applicants took two approaches to perturbing it in the context of CNS autoimmune disease, EAE: chemical inhibition of ODC1 and T-cell specific genetic deletion of SAT1 (FIG. 37). As targeting multiple nodes in the polyamine pathway resulted in upregulation of Foxp3 during Th17 differentiation in vitro (FIGS. 35 and 36), Applicants hypothesized that targeting rate-limiting enzymes in polyamine pathway in vivo would regulate induction of EAE.

Applicants first tested ODC1 inhibition by adding DFMO in the drinking water for mice immunized with MOG/CFA for the induction of EAE (STAR Methods). DFMO significantly delayed the onset and severity of EAE (FIG. 37B). Consistently, Applicants observed a significantly reduced antigen-specific recall response in the draining lymph node of DFMO treated animals (FIG. 37C). Further analysis of lymphocytes isolated from the CNS showed no difference in the frequency of cytokine producing cells, but increased frequency of Foxp3⁺ CD4⁺ T cells out of all CD4 T cells (FIG. 37D and data not shown), consistent with the polyamine biosynthesis pathway as an important positive regulator of the balance between proinflammatory Th17 cells and Foxp3⁺ Tregs and induction of autoimmune CNS inflammation, which is highly dependent on Th17 cells.

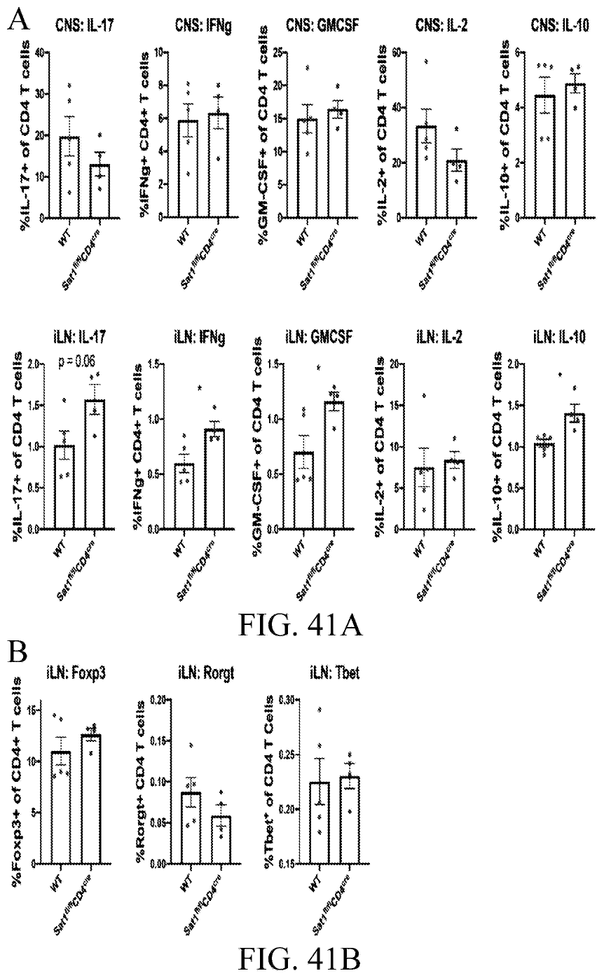

Since administering DFMO in the drinking water could affect multiple cell types, Applicants also genetically deleted SAT1, the rate limiting enzyme of the polyamine pathway, in CD4⁺ T cells (SAT1^{fl/fl}CD4^{cre}). Applicants confirmed that genetic deletion of SAT1 in T cells resulted in loss of polyamine acetylation as reflected in reduced levels of acetyl-putrescine and acetyl-spermidine (FIG. 37E). Notably, loss of SAT1 also resulted in reduced level of putrescine in Th17 cells, likely through a feedback mechanism. This is consistent with reports in other cell types [31] and the in vitro inhibitor data (FIG. 39), suggesting similar effect of DFMO and SAT1 deletion in the context of T cell biology may be due to overall changes in polyamine flux. Indeed, Applicants observed significantly delayed onset and severity of EAE in SAT1^{fl/fl}CD4^{cre} mice (FIG. 37F). Similar to global inhibition of ODC1 by DFMO treatment, Applicants observed an inhibition of antigen-specific recall responses as measured by T cell proliferation (FIG. 37G). Although Applicants did not observe significant differences in cytokine production (FIGS. 3711 and 41A), there was a trend towards a decrease in IFN-g, IL-17 and TNF production with an increase in IL-9 production in response to antigen (FIG. 3711). Furthermore, there was a significant increase in the proportion of Foxp3⁺CD4⁺ T cells (out of all CD4 T cells) and concomitant decrease of Rorgt⁺CD4⁺ T cells isolated from the target organ (CNS) of SAT1^{fl/fl}CD4^{cre} mice (FIG. 37I). Notably, the frequencies of Foxp3+ or Rorgt+ cells are not different in the draining lymph node (FIG. 41B), suggesting that the effect of SAT1 on T cells may be amplified in tissue recall responses. Thus, using both chemical and genetic perturbations at multiple levels, Applicants demonstrated that the polyamine pathway is an important mediator of autoimmune inflammation.

### Discussion

To understand the functional relevance of metabolic pathways in Th17 cells, Applicants utilized metabolomics, a novel computational algorithm (Compass, Wagner et al., 2020, Example 9) and chemical and genetic perturbation to investigate the functional metabolic networks that impact Th17 pathogenicity. In this study, Applicants investigated in depth the metabolic circuitry centered around the polyamine pathway. Applicants demonstrated that 1) At the transcriptome level, Compass points to the significance of the polyamine pathway as a top candidate in association with Th17 cell pathogenicity and implicates reactions upstream and downstream of putrescine to be associated with functional phenotype of Th17 cells; 2) As predicted by Compass and measured by enzymatic assay and LC/MS metabolomics, Applicants showed that Th17 cells at different functional state have alternative metabolic flux anchored around arginine and putrescine, the precursor to polyamines, and that both regulatory T cells and non-pathogenic Th17 cell have reduced cellular content of polyamines; 3) Chemical targeting of multiple enzymes in the polyamine pathway and genetic deletion of ODC1 resulted in suppression of the Th17 functional program and upregulation of Foxp3 in a putrescine dependent manner; 4) Inhibiting polyamine biosynthesis shifts Th17 cells in favor of Treg-like transcriptome and epigenome; 5) Targeting ODC1 and SAT1 both resulted in upregulation of Foxp3 in vivo and inhibition of effector Th17 cells and regulation of EAE. Taken together, Applicants have provided evidence supporting a critical role of the polyamine pathway in suppressing regulatory program in Th17 cells.

Th17 cells are critical in inducing autoimmune inflammation. In fact, loss of all the components in Th17 pathway including TGF-b, IL-6, IL-1 or IL-23 results in inhibition of Th17 differentiation, upregulation of FoxP3+ Tregs and suppression of EAE. Because of reciprocal generation of Tregs vs. Th17 cells, the effects observed with the inhibition of polyamine pathway may be unique to the diseases where Th17 cells are the effector cells. Whether the effect of polyamine pathway can be generalized to other autoimmune diseases (e.g. autoimmune colitis or type 1 diabetes), where Th1 or NK cells are the effectors, need to be further evaluated. In fact the effects of blocking polyamine pathway in diverting Th17 differentiation to Treg phenotype was much more profound in generating nonpathogenic Th17 (differentiation with TGFb) than in pathogenic Th17 cells (differentiation with IL-1b and IL-23). This observation suggests that inhibition of the pathway may have an effect that is unique to Th17 driven diseases.

The significance of the polyamine pathway in autoimmune diseases is further supported by anecdotal data that polyamine levels are increased in several autoimmune diseases [34, 35] and it is thought that aberrant polyamine metabolism can contribute to autoantigen stabilization [36]. Here Applicants present a potential mechanism of how the polyamine pathway can regulate Th17/Treg balance and impact development of autoimmunity. DFMO is an FDA-approved drug for cancer therapy. Applicants showed that DFMO has significant impact in curtailing EAE, providing the grounds/mechanism for drug repurposing. It should be noted that while targeting any enzyme in the polyamine pathway resulted in similar effects in Th17 cells in vitro, genetic manipulation of ODC1 and SAT1 are not identical in that while both ODC1 and SAT1 deletion promoted Foxp3 expression (FIG. 35I and data not shown), ODC1 but not SAT1 suppressed Th17 cytokine expression in vitro (FIG. 35I and data not shown). Further studies are necessary to understand the mechanistic difference within the polyamine pathway.

By studying the metabolic differences within the same lineage of effector Th17 cells, Applicants unexpectedly uncovered a central role of the polyamines in regulating Th17-Treg balance. This study suggests a functional role of metabolic pathways beyond energy production. One of the observations made in this study is the role that polyamine pathway plays in shaping the epigenetic landscape of differentiating immune cells. In fact, looking at the ATACseq and RNAseq profiles of Th17 cells activated in the presence of inhibitors of the polyamine pathway shows profound global ATACseq changes concomitantly with changes in transcription, differentiation and function. Polyamines appear to regulate gene expression, cell proliferation and stress responses due to their ability to bind to nucleic acids (both DNA, RNA), alter posttranslational modification and regulate ion channels [37, 38]. A number of studies have suggested the role of polyamines in regulating gene expression due to their polycationic nature and ability to function as a sink to S-adenosylmethionine and Acetyl-coA, both critical metabolites for histone modifications [29, 30, 39, 40]. Furthermore, intracellular polyamines and their analogues are also known to inhibit lysine-specific demethyltransferases such as LSD1 [41] and thereby changing epigenetic landscape affecting development and differentiation. Thus, it stands to reason that metabolic processes that impact polyamines will not only affect energetics but more broadly including shaping the epigenome and transcriptome by the resultant metabolites that are produced during the process of development or differentiation. In this vein, a number of developmental disorders (eg., Snyder-Robinson syndrome) have been associated with maladapted polyamine metabolism [42].

It is very clear that when immune cells take up residence in different tissues they also change their transcriptomes and attain specialized or different functions. Notable examples of this issue has been shown in tissue Tregs [43] and macrophages [44], where the cells look very different transcriptomically depending on the tissue of residence. Applicants and others have observed a similar situation in Th17 cells, where they differ in their function of whether they are in lymph nodes, gut or CNS, as observed by the scRNAseq analysis of Th17 cells [23, 24]. Based on the studies, presented here, Applicants suggest that the metabolic activity of the cell within a defined tissue may have a profound impact in the epigenome and transcriptome, resulting in their changed or specialized functions. With the emerging cell atlases and mapping transcriptome of tissues resident immune cells at the single cell level, the Compass algorithm will provide a powerful tool for studying metabolic pathways across different cell types in different tissues, taking advantage of the wealth of single cell data sets that are being published.

In summary, this study highlights the advantage of utilizing single cell genomics and novel algorithms in studying cellular metabolism, providing roadmaps for studying metabolic pathways in immune cells across normal or diseased tissues. The study validates the predictions made by algorithms, both in vitro and in vivo and shows that interfering with these metabolic pathways identified by Compass have profound effect on the function of the effector cells, by regulating both epigenome and transcriptome of the Th17 cell.

### Example 8 Tables, see Wang et al. 2020.

### Example 8 STAR Methods

Mice. C57BL/6 wildtype (WT) were obtained from Jackson laboratory (Bar Harbor, Me.). SAT1flox mice were kindly provided by Dr. Manoocher Soleimani (University of Cincinnati), which Applicants crossed to CD4cre to generate conditional T cell deletion of SAT1. Note that only male mice were used in all experiments as SAT1 is an X chromosome gene and female mice have incomplete deletion due to random inactivation of x chromosome. ODC1^{fl/fl}CD4^{cre} were gifted by Dr. Erika Pearce (Max Planck Institute). For experiments, mice were matched for sex and age, and most mice were 6-10 weeks old. Littermate WT or Cre− mice were used as controls. All experiments were conducted in accordance with animal protocols approved by the Harvard Medical Area Standing Committee on Animals or BWH IACUC.

T cell differentiation culture and flow cytometry. Naïve CD4+CD44-CD62L+CD25− T cells were sorted using BD FACSAria sorter and activated with plate-bound anti-CD3 (1 μg/ml) and antiCD28 antibodies (1 μg/ml) in the presence of cytokines at a concentration of 5×10⁵ cells/ml. For T cell differentiations the following combinations of cytokines were used: pathogenic Th17: 25 ng/ml rmIL-6, 20 ng/ml rmIL-1b (both Miltenyi Biotec) and 20 ng/ml rmIL-23 (R&D systems); non-pathogenic Th17: 25 ng/ml rmIL-6 and 2 ng/ml of rhTGFb1 (Miltenyi Biotec); iTreg: 2 ng/ml of rhTGFb1; Th1: 20 ng/ml rmIL-12 (R&D systems); Th2: 20 ng/ml rmIL-4 (Miltenyi Biotec). Intracellular cytokine staining was performed after incubation for 4-6h with Cell Stimulation cocktail plus Golgi transport inhibitors (Thermo Fisher Scientific) using the BD Cytofix/Cytoperm buffer set (BD Biosciences) per manufacturer's instructions. Transcription factor staining was performed using the Foxp3/Transcription Factor Staining Buffer Set (eBioscience). Proliferation was assessed by staining with CellTrace Violet (Thermo Fisher Scientific) per manufacturer's instructions. Apoptosis was assessed using Annexin V staining kit (BioLegend). Phosphorylation of proteins to determine cell signaling was performed with BD Phosflow buffer system (BD bioscience) as per manufacturer's instructions.

Inhibitors and metabolites. For differentiation experiments, cells were harvested at 72 hours and were performed in the presence or absence of 100-200 μM DFMO, 500 μM trans-4-Methylcyclohexylamine (MCHA, both Sigma), 500 μM N-(3-Aminopropyl)cyclohexylamine (APCHA, Santa Cruz Biotechnology), 50 μM Diminazene aceturate (Dize, Cayman Chemical) with or without 2.5 mM Putrescine (Sigma, P7505) as indicated.

Compass analysis. Compass is descried in detail in Example 9. In the following Applicants provide a high level description of the algorithm.

Compass integrates scRNA-Seq profiles with prior knowledge of the metabolic network to infer a metabolic state of the cell. The metabolic network Applicants use here consists of 7,440 reactions and 2,626 metabolites (Recon2 database, [27]), along with reaction stoichiometry, gene-enzyme-reaction associations and biochemical constraints (such as reaction irreversibility and nutrient availability).

Compass builds on the paradigm of Flux Balance Analysis (FBA) to model metabolic fluxes, namely the rate by which chemical reactions convert substrates to products [25, 26, 45, 46] (Orth, Thiele, and Palsson 2010; O'Brien, Monk, and Palsson 2015; Lewis, Nagarajan, and Palsson 2012; Palsson 2015). The modeling is based on linear programming, maximizing a certain objective (here, flux through a given reaction), while using the metabolic network to pose constraints.

In its first step, Compass is agnostic to any measurement of gene expression levels and computes, for every metabolic reaction r, the maximal flux vᵣ^{opt} it can carry without imposing any constraints on top of those imposed by stoichiometry and mass balance. Next, Compass assigns every reaction in every cell a penalty inversely proportional to the mRNA expression associated with its enzyme(s) in that cell. Compass then finds a flux distribution which minimizes the overall penalty incurred in any given cell i (summing over all reactions), while maintaining a flux of at least 0.95·vᵣ^{opt} in r. The Compass score of reaction r in cell i is the negative of that minimal penalty (so that lower scores correspond to lower potential metabolic activity). Intuitively, these scores reflect how well adjusted is each cell's transcriptome to maintaining high flux through each reaction. To reduce the effects of data sparsity (characteristic of scRNA-Seq) Compass uses an information-sharing approach. Instead of treating each cell in isolation, the score vector for each cell is determined by a combined objective that balances the effects in the cell in question with those in its ten nearest neighbors (based on similarity of their RNA profiles).

After applying Compass to the scRNA-Seq of Th17 cells, Applicants aggregated reactions that were highly correlated across the entire dataset (Spearman rho>0.98) into meta-reactions (with median of two reactions per meta-reaction) for downstream analysis. For the ranking analysis in FIG. 34C, Applicants prioritized meta-reactions with differential predicted activity between the Th17p and Th17n conditions. To this end, Applicants used Wilcoxon's rank sum p-values (comparing Th17 cells differentiated under the non-pathogenic conditions vs. Th17 cells differentiated under the pathogenic conditions) and Spearman rank correlation (correlating reaction scores with pathogenicity scores across cells).

qPCR. RNA was isolated using RNeasy Plus Mini Kit (Qiagen) and reverse transcribed to cDNA with iScript cDNA Synthesis Kit (Bio-Rad). Gene expression was analyzed by quantitative real-time PCR on a ViiA7 System (Thermo Fisher Scientific) using TaqMan Fast Advanced Master Mix (Thermo Fisher Scientific) with the following primer/probe sets: Ass1 (Mm00711256_m1), Odc1 (Mm02019269_g1), Sat1 (Mm00485911_g1), Srm (Mm00726089_s1), Sms (Mm00786246_s1), Il-17a (Mm00439618_m1), Il-17f (Mm00521423_m1), Foxp3 (Mm00475162_m1), Tead1 (Mm00493507_m1), Taz (Mm00504978_m1), and Actb (Applied Biosystems). Expression values were calculated relative to Actb detected in the same sample by duplex qPCR.

Polyamine ELISA. Cell pellets of in vitro differentiated cells were frozen down and further processed with the Total Polyamine Assay Kit (BioVision Inc.) according to the manufacturer's instructions.

Metabolomics/Carbon tracing. For untargeted metabolomics, Th17 cells were differentiated as described. Culture media were snap frozen. Cells were harvested at 96h. 1×10⁶ cells per sample were snap frozen and extracted in either 80% methanol (for fatty acids and oxylipids) or isopropanol (for polar and nonpolar lipids). Two liquid chromatography tandem mass spectrometry (LC-MS) methods were used to measure fatty acids and lipids in cell extracts.

For carbon tracing experiments Th17 cells were differentiated as described. At 48h, cells were washed and cultured in media supplemented with Arginine (¹³C6, Sigma, Cat #643440) or aspartic acid (¹³C4, Sigma, Cat #604852) for 1, 5 and 24 hours.

Legendplex. Cytokine concentrations in supernatants of in vitro cultures were analyzed by the LegendPlex Mouse Th Cytokine Panel (13-plex) (BioLegend) according to the manufacturer's instructions and analyzed on a FACS LSR II (BD Biosciences).

RNA-seq. For population (bulk) RNA-seq, in vitro differentiated T-cells were sorted for live cells and lysed with RLT Plus buffer and RNA was extracted using the RNeasy Plus Mini Kit (Qiagen). Full-length RNA-seq libraries were prepared as previously described [47] and paired-end sequenced (75 bp×2) with a 150 cycle Nextseq 500 high output V2 kit.

Bioinformatic analysis of RNA-seq data. Alignment, quantification, and computation of pathogenicity signatures based on single-cell transcriptomes were conducted as described in the cosubmitted manuscript (Wagner et al.). Briefly, raw scRNA-seq reads from Gaublomme et al. (2015) [24] (FIG. 34) were aligned with Bowtie2, quantified into TPM gene expression with RSEM. Quality control tested and batch effects and other nuisance factors removed with SCONE [48].

To compute a pathogenicity score for each cell Applicants used a similar scheme as in [24]: For each cell Applicants take the average z-scored normalized log expression of pro-pathogenic markers (CASP1, CCL3, CCL4, CCL5, CSF2, CXCL3, GZMB, ICOS, IL22, IL7R, LAG3, LGALS3, LRMP, STAT4, TBX21) and of pro-regulatory markers (AHR, IKZF3, IL10, IL1RN, IL6ST, IL9, MAF), with the latter multiplied by−1.

Bulk RNA libraries from DFMO- or vehicle-treated Th17p, Th17n, or Treg were studied with 3 replicates per condition for a total of 18 libraries as shown in FIG. 36A. Genes that are associated with a Th17 or Treg programs (orange and purple, respectively, in FIG. 36B-C) were determined by differential expression test between bulk RNA libraries of (vehicle-treated) Th17n and Th17p on one side and Treg on the other with BH-adjusted p<0.05 and absolute value of log 2 fold-change of at least 1.5. Genes associated with the Th17p or Th17n program (magenta and green, respectively, in FIG. 40A) were determined by differential expression test between bulk RNA libraries of (vehicle-treated) Th17p vs. Th17n with the same thresholds. The PCA shown in FIG. 36A was computed on the set of 3,414 that were differentially associated with Th17, Treg, Th17p, or Th17n programs by the aforementioned criteria to focus it on the subspace of the transcriptome relevant to Th17 pathogenicity phenotypes.

ATAC-seq. For population ATAC-seq, in vitro differentiated T-cells were sorted for live cells and stored in Bambanker freezing media (Thermo Fisher Scientific) at −80° C. until further processing. Prior to library preparation, cells were thawed at 37° C. and washed with PBS. For ATAC-seq, cell pellets were lysed and tagmented in 1×TD Buffer, 0.2 ul TDE1 (Illumina), 0.01% digitonin, and 0.3×PBS in 40 ul reaction volume following the protocol described in [49]. Transposition reactions were incubated at 37° C. for 30 min at 300 rpm. The DNA was purified from the reaction using a MinElute PCR purification kit (QIAGEN). The whole resulting product was then PCR-amplified using indexed primers with NEBNext High-Fidelity 2X PCR Master Mix (NEB). First, Applicants performed 5 cycles of pre-amplification. Applicants sampled 10% of the pre-amplification reaction for SYBR Green quantitative PCR to assess the number of additional cycles needed for final amplification. After purifying the final library with the MinElute PCR purification kit (QIAGEN), the library was quantified for sequencing using qPCR and a Qubit dsDNA HS Assay kit (Invitrogen). Libraries were sequenced on an Illumina NextSeq 550 system with paired-end reads of 37 base pairs in length.

Alignment of ATAC-Seq and Peak Calling. All ATAC-Seq reads were trimmed using Trimmomatic [50] to remove primer and low-quality bases. Reads <36 bp were dropped. Reads were then passed to FastQC [www.bioinformatics.babraham.ac.uk/projects/fastqc/] to check the quality of the trimmed reads. The paired-end reads were then aligned to the mm10 reference genome using bowtie2 [51], allowing maximum insert sizes of 2000 bp, with the “—no-mixed” and “—no-discordant” parameters added. Reads with a mapping quality (MAPA) below 30 were removed. Duplicates were removed with PicardTools, and the reads mapping to the blacklist regions and mitochondrial DNA were also removed. Reads mapping to the positive strand were moved +4 bp, and reads mapping to the negative strand were moved −5 bp following the procedure outlined in [52] to account for the binding of the Tn5 transposase.

Peaks were called using macs2 on the aligned fragments [53] with a qvalue cutoff of 0.001 and overlapping peaks among replicates were merged.

Tests of Differential Accessibility. Differential accessibility was assessed using DESeq2 [54] on with a matrix of peaks (merging all samples) by samples. Similar to common practice in the analysis of differential gene expression, the analysis of differential accessibility was conducted using the number of observed Tn5 cuts (i.e., number of reads).

Peaks that are associated with a Th17 or Treg programs (orange and purple, respectively, in FIG. 36D) were determined by differential accessibility test between libraries of (vehicle-treated) Th17n and Th17p on one side (unpublished dataset) and Treg on the other with BH-adjusted p<0.05 and absolute value of log 2 fold-change of at least 1.

Reprocessing of published ChIP-Seq data. ChIP-Seq Peaks from Xiao et al 2014 [32] were transferred from mm9 to mm10 using the UCSC liftOver tool. ChIP-Seq replicates from Ciofani et al 2012 were downloaded and were trimmed using Trimmomatic [26] to remove primer and low-quality bases. Reads were then passed to FastQC [www.bioinformatics.babraham.ac.uk/projects/fastqc/] to check the quality of the trimmed reads. These single-end reads were then aligned to the mm10 reference genome using bowtie2 [27], allowing maximum insert sizes of 2000 bp, with the “—no-mixed” and “—no-discordant” parameters added. Reads with a mapping quality (MAPA) below 30 were removed. Duplicates were removed with PicardTools, and the reads mapping to the blacklist regions and mitochondrial DNA were also removed.

ChIP-Seq peaks were called in each replicate, versus a control sample, using macs2 [29] with a qvalue cutoff of 0.05.

Enrichment of motifs and ChIP-seq peaks in differentially accessible regions. Peaks were considered differentially accessible if they had a BH-adjusted p<0.05. Applicants calculated fold enrichment of various genomic features in these peaks (described below) versus a background set of peaks. q-values were estimated using q-value package. [Storey J D, Bass A J, Dabney A, Robinson D. qvalue: Q-value estimation for false discovery rate control. github.com/jdstorey/qvalue]

Motifs/Annotation Tracks. PWM's for motifs were downloaded from the 2018 release of JASPAR [55, 56]. Applicants used fimo [56] to identify motifs in mm10, and applied the default threshold of 1e-4. Applicants also included regulatory features from the ORegAnno database[57], (iii) conserved regions annotated by the multiz30way algorithm, and repeat regions annotated by RepeatMasker (www.repeatmasker.org).

GREAT Pathways/Genes. Loci were associated with pathways using GREAT[58], submitted with the rGREAT package (github.com/jokergoo/rGREAT). Applicants retrieved pathways found in the MSigDB Immunologic Signatures, MSigDB Pathway, and GO Biological Process databases. Loci were mapped to genes using GREAT.

Experimental Autoimmune Encephalomyelitis (EAE). For active EAE immunization, MOG35-55 peptide was emulsified in complete freund adjuvant (CFA). Equivalent of 40 μg MOG peptide was injected per mouse subcutaneously followed by pertussis toxin injection intravenously on day 0 and day 2 of immunization. Mice were treated with 0.5% DFMO in drinking water for 10 days as indicated. DFMO was replenished every third day.

Statistical Analysis. Unless otherwise specified, all statistical analyses were performed using the two-tail student t test using GraphPad Prism software. P value less than 0.05 is considered significant (P<0.05=*; P<0.01=**; P<0.001=***) unless otherwise indicated.

### Example 8 References

1. Kleinewietfeld, M. and D. A. Hafler, The plasticity of human Treg and Th17 cells and its role in autoimmunity. Semin Immunol, 2013. 25(4): p. 305-12.
2. Noack, M. and P. Miossec, Th17 and regulatory T cell balance in autoimmune and inflammatory diseases. Autoimmun Rev, 2014. 13(6): p. 668-77.
3. Bettelli, E., et al., Reciprocal developmental pathways for the generation of pathogenic effector TH17 and regulatory T cells. Nature, 2006. 441(7090): p. 235-8.
4. Mangan, P. R., et al., Transforming growth factor-beta induces development of the T(H)17 lineage. Nature, 2006. 441(7090): p. 231-4.
5. Veldhoen, M., et al., TGFbeta in the context of an inflammatory cytokine milieu supports de novo differentiation of IL-17-producing T cells. Immunity, 2006. 24(2): p. 179-89.
6. McGeachy, M. J. and D. J. Cua, Th17 cell differentiation: the long and winding road. Immunity, 2008. 28(4): p. 445-53.
7. Guglani, L. and S. A. Khader, Th17 cytokines in mucosal immunity and inflammation. Curr Opin HIV AIDS, 2010. 5(2): p. 120-7.
8. Ouyang, W., J. K. Kolls, and Y. Zheng, The biological functions of T helper 17 cell effector cytokines in inflammation. Immunity, 2008. 28(4): p. 454-67.
9. Bettelli, E., et al., Induction and effector functions of T(H)17 cells. Nature, 2008. 453(7198): p. 1051-7.
10. Korn, T., et al., IL-17 and Th17 Cells. Annu Rev Immunol, 2009. 27: p. 485-517.
11. Gaffen, S. L., N. Hernandez-Santos, and A. C. Peterson, 17 signaling in host defense against Candida albicans. Immunol Res, 2011. 50(2-3): p. 181-7.
12. Romani, L., Immunity to fungal infections. Nat Rev Immunol, 2011. 11(4): p. 275-88.
13. Jager, A., et al., Th1, Th17, and Th9 effector cells induce experimental autoimmune encephalomyelitis with different pathological phenotypes. J Immunol, 2009. 183(11): p. 7169-77.
14. Lee, Y., et al., Induction and molecular signature of pathogenic TH17 cells. Nat Immunol, 2012. 13(10): p. 991-9.
15. McGeachy, M. J., et al., TGF-beta and IL-6 drive the production of IL-17 and IL-10 by T cells and restrain T(H)-17 cell-mediated pathology. Nat Immunol, 2007. 8(12): p. 1390-7.
16. Awasthi, A., et al., Cutting edge: IL-23 receptor gfp reporter mice reveal distinct populations of IL-17-producing cells. J Immunol, 2009. 182(10): p. 5904-8.
17. Cua, D. J., et al., Interleukin-23 rather than interleukin-12 is the critical cytokine for autoimmune inflammation of the brain. Nature, 2003. 421(6924): p. 744-8.
18. McGeachy, M. J., et al., The interleukin 23 receptor is essential for the terminal differentiation of interleukin 17-producing effector T helper cells in vivo. Nat Immunol, 2009. 10(3): p. 314-24.
19. Chung, Y., et al., Critical regulation of early Th17 cell differentiation by interleukin-1 signaling. Immunity, 2009. 30(4): p. 576-87.
20. Ghoreschi, K., et al., Generation of pathogenic T(H)17 cells in the absence of TGF-beta signalling. Nature, 2010. 467(7318): p. 967-71.
21. Lee, Y., M. Collins, and V. K. Kuchroo, Unexpected targets and triggers of autoimmunity. J Clin Immunol, 2014. 34 Suppl 1: p. S56-60.
22. Zielinski, C. E., et al., Pathogen-induced human TH17 cells produce IFN-gamma or IL-10 and are regulated by IL-1beta. Nature, 2012. 484(7395): p. 514-8.
23. Wang, C., et al., CDSL/AIM Regulates Lipid Biosynthesis and Restrains Th17 Cell Pathogenicity. Cell, 2015. 163(6): p. 1413-27.
24. Gaublomme, J. T., et al., Single-Cell Genomics Unveils Critical Regulators of Th17 Cell Pathogenicity. Cell, 2015. 163(6): p. 1400-12.
25. Orth, J. D., I. Thiele, and B. O. Palsson, What is flux balance analysis? Nat Biotechnol, 2010. 28(3): p. 245-8.
26. O'Brien, E. J., J. M. Monk, and B. O. Palsson, Using Genome-scale Models to Predict Biological Capabilities. Cell, 2015. 161(5): p. 971-987.
27. Thiele, I., et al., A community-driven global reconstruction of human metabolism. Nat Biotechnol, 2013. 31(5): p. 419-25.
28. Bowlin, T. L., B. J. McKown, and P. S. Sunkara, The effect of alpha-difluoromethylornithine, an inhibitor of polyamine biosynthesis, on mitogen-induced interleukin 2 production. Immunopharmacology, 1987. 13(2): p. 143-7.
29. Jell, J., et al., Genetically altered expression of spermidine/spermine N1-acetyltransferase affects fat metabolism in mice via acetyl-CoA. J Biol Chem, 2007. 282(11): p. 8404-13.
30. Pegg, A. E., Spermidine/spermine-N(1)-acetyltransferase: a key metabolic regulator. Am J Physiol Endocrinol Metab, 2008. 294(6): p. E995-1010.
31. Mounce, B. C., et al., Interferon-Induced Spermidine-Spermine Acetyltransferase and Polyamine Depletion Restrict Zika and Chikungunya Viruses. Cell Host Microbe, 2016. 20(2): p. 167-77.
32. Xiao, S., et al., Small-molecule RORgammat antagonists inhibit T helper 17 cell transcriptional network by divergent mechanisms. Immunity, 2014. 40(4): p. 477-89.
33. Li, Q., et al., Critical role of histone demethylase Jmjd3 in the regulation of CD4+ T-cell differentiation. Nat Commun, 2014. 5: p. 5780.
34. Karouzakis, E., et al., Increased recycling of polyamines is associated with global DNA hypomethylation in rheumatoid arthritis synovial fibroblasts. Arthritis Rheum, 2012. 64(6): p. 1809-17.
35. Hsu, H. C., J. R. Seibold, and T. J. Thomas, Regulation of ornithine decarboxylase in the kidney of autoimmune mice with the lpr gene. Autoimmunity, 1994. 19(4): p. 253-64.
36. Brooks, W. H., Increased polyamines alter chromatin and stabilize autoantigens in autoimmune diseases. Front Immunol, 2013. 4: p. 91.
37. Pegg, A. E., Mammalian polyamine metabolism and function. IUBMB Life, 2009. 61(9): p. 880-94.
38. Pegg, A. E., Functions of Polyamines in Mammals. J Biol Chem, 2016. 291(29): p. 14904-12.
39. Kraus, D., et al., Nicotinamide N-methyltransferase knockdown protects against diet-induced obesity. Nature, 2014. 508(7495): p. 258-62.
40. Childs, A. C., D. J. Mehta, and E. W. Gerner, Polyamine-dependent gene expression. Cell Mol Life Sci, 2003. 60(7): p. 1394-406.
41. Tamari, K., et al., Polyamine flux suppresses histone lysine demethylases and enhances IDI expression in cancer stem cells. Cell Death Discov, 2018. 4: p. 104.
42. Murray-Stewart, T., et al., Polyamine Homeostasis in Snyder-Robinson Syndrome. Med Sci (Basel), 2018. 6(4).
43. Cipolletta, D., et al., Tissular T(regs): a unique population of adipose-tissue-resident Foxp3+CD4+ T cells that impacts organismal metabolism. Semin Immunol, 2011. 23(6): p. 431-7.
44. Epelman, S., K. J. Lavine, and G. J. Randolph, Origin and functions of tissue macrophages. Immunity, 2014. 41(1): p. 21-35.
45. Lewis, N. E., H. Nagarajan, and B. O. Palsson, Constraining the metabolic genotype-phenotype relationship using a phylogeny of in silico methods. Nat Rev Microbiol, 2012. 10(4): p. 291-305.
46. Palsson, B. O., Systems Biology: Constraint-Based Reconstruction and Analysis. 2nd ed.edition. 2015: Cambridge University Press.
47. Singer, M., et al., A Distinct Gene Module for Dysfunction Uncoupled from Activation in Tumor-Infiltrating T Cells. Cell, 2016. 166(6): p. 1500-1511 e9.
48. Cole, M. B., et al., Performance Assessment and Selection of Normalization Procedures for Single-Cell RNA-Seq. Cell Syst, 2019. 8(4): p. 315-328 e8.
49. Corces, M. R., et al., Lineage-specific and single-cell chromatin accessibility charts human hematopoiesis and leukemia evolution. Nat Genet, 2016. 48(10): p. 1193-203.
50. Bolger, A. M., M. Lohse, and B. Usadel, Trimmomatic: a flexible trimmer for Illumina sequence data. Bioinformatics, 2014. 30(15): p. 2114-20.
51. Langmead, B. and S. L. Salzberg, Fast gapped-read alignment with Bowtie 2. Nat Methods, 2012. 9(4): p. 357-9.
52. Buenrostro, J. D., et al., Transposition of native chromatin for fast and sensitive epigenomic profiling of open chromatin, DNA-binding proteins and nucleosome position. Nat Methods, 2013. 10(12): p. 1213-8.
53. Zhang, Y., et al., Model-based analysis of ChIP-Seq (MACS). Genome Biol, 2008. 9(9): p. R137.
54. Love, M. I., W. Huber, and S. Anders, Moderated estimation of fold change and dispersion for RNA-seq data with DESeq2. Genome Biol, 2014. 15(12): p. 550.
55. Khan, A., et al., JASPAR 2018: update of the open-access database of transcription factor binding profiles and its web framework. Nucleic Acids Res, 2018. 46(D1): p. D260-D266.
56. Grant, C. E., T. L. Bailey, and W. S. Noble, FIMO: scanning for occurrences of a given motif. Bioinformatics, 2011. 27(7): p. 1017-8.
57. Lesurf, R., et al., ORegAnno 3.0: a community-driven resource for curated regulatory annotation. Nucleic Acids Res, 2016. 44(D1): p. D126-32.
58. McLean, C. Y., et al., GREAT improves functional interpretation of cis-regulatory regions. Nat Biotechnol, 2010. 28(5): p. 495-501.

### Example 9—in Silico Modeling of Metabolic State in Single Th17 Cells Reveals Novel Regulators of Inflammation and Autoimmunity

Cellular metabolism is a major regulator of immune response, but it is difficult to study the metabolic status of an individual immune cell using current technologies. Here, Applicants present Compass, an algorithm to characterize the metabolic landscape of single cells in silico based on single-cell RNA-Seq profiles and flux balance analysis. Applicants used Compass to study the landscape of metabolic heterogeneity in T helper 17 (Th17) cells and predict novel metabolic regulators of their inflammatory function. Compass recovered the known metabolic switch between glycolysis and fatty acid oxidation and further predicted novel regulators in amino-acid pathways, which Applicants validated through transcriptomic, metabolic and functional assays. Compass also predicted a particular glycolytic reaction (phosphoglycerate mutase—PGAM) that promotes an anti-inflammatory phenotype in Th17 cells, contrary to common immunometabolic understanding of a shift towards glycolysis as promoting pro-inflammatory phenotypes in Th17 and other immune cell types. Applicants validate this prediction and demonstrate that enzymatic inhibition of PGAM leads non-pathogenic Th17 cells to adopt a pro-inflammatory transcriptional program and develop an encephalitogenic phenotype with autoimmunity of the central nervous system upon adoptive transfer in vivo. As diverse cells are profiled by single-cell RNA-Seq in efforts such as the Human Cell Atlas, Compass offers the first broadly applicable tool to characterize metabolic state of individual cells, relate their metabolic state to the transcriptomes and cellular phenotypes, and correlate the state to drivers regulating the phenotype.

### Introduction

Cellular metabolism is both a mediator and a regulator of cellular functions. Metabolic activities are key in normal cellular processes such as activation, expansion and differentiation, but also play an important role in the pathogenesis of multiple disease conditions including autoimmunity, cancer, cardiovascular disease, neurodegeneration, and aging. Recently, the study of metabolism in immune cells (immunometabolism) has gained particular attention as a major regulator of almost all aspects of immune responses including anti-viral immunity, autoimmunity, and cancer (Hotamisligil 2017; O'Neill, Kishton, and Rathmell 2016; Geltink, Kyle, and Pearce 2018; Russell, Huang, and VanderVen 2019; Buck et al. 2017; Ho and Kaech 2017; Chapman, Boothby, and Chi 2019; Karmaus et al. 2019; Green, Galluzzi, and Kroemer 2014).

Due to the scale and complexity of the metabolic network, a metabolic perturbation may create cascading effects and eventually alter a seemingly distant part of the network, while cross-cutting traditional pathway definitions. Therefore, computational tools are needed contextualize observations on specific reactions or enzymes into a systems-level understanding of metabolism and its dysregulation in disease. One successful framework has been Flux Balance Analysis (FBA), which translates curated knowledge on the network's topology and stoichiometry into mathematical objects and uses them to make in silico predictions on metabolic fluxes (Orth, Thiele, and Palsson 2010; O'Brien, Monk, and Palsson 2015; Lewis, Nagaraj an, and Palsson 2012; Palsson 2015). FBA methods have proven particularly useful when contextualized with functional genomics data, including gene expression (Bordbar et al. 2014).

While such metabolic models aim to represent the behavior of individual cells, their contextualization has generally relied on information collected from bulk population data. However, the advent of single-cell RNA-Seq (scRNA-Seq) has highlighted the substantial extent of cell-to-cell diversity that is often missed by bulk profiles (A. Wagner, Regev, and Yosef 2016; Tanay and Regev 2017), and can be especially prominent in immune cells and associated with their functional diversity (Ben-Moshe et al. 2019; Vieira Braga et al. 2019; Karmaus et al. 2019; Azizi et al. 2018; Sade-Feldman et al. 2018; Keren-Shaul et al. 2017; Vento-Tormo et al. 2018; Paul et al. 2015; Soldatov et al. 2019; Miragaia et al. 2019; Zanini et al. 2018; Brown et al. 2019; Van Hove et al. 2019). One of the earliest examples has been the diversity among T helper 17 (Th17) cells (Gaublomme et al. 2015). On the one hand, IL-17 producing Th17 cells can be potent inducers of tissue inflammation in autoimmune disorders (Korn et al. 2009; Tesmer et al. 2008) but on the other hand, these cells are critical in host defense against pathogens (Gaffen, Hernandez-Santos, and Peterson 2011; Romani 2011) and can promote mucosal homeostasis and barrier functions (Lee et al. 2012; Stockinger and Omenetti 2017; X. Wu, Tian, and Wang 2018). Th17 cells with distinct effector functions can be found in patients and animal models and can also be generated in vitro with different combinations of differentiation cytokines, as Applicants have previously demonstrated (Lee et al. 2012). Applicants have previously shown that such functional diversity can be captured by studying transcriptional diversity at the single cell level with scRNA-Seq, and enabled the discovery of novel regulators that are otherwise difficult to detect in bulk RNA-Seq analysis (Gaublomme et al. 2015; Wang et al. 2015).

Applicants hypothesized that a similar spectrum of diversity may exist at the immunometabolic level and relate to cell function. However, most cellular assays, including metabolic assays, are normally done in a targeted manner and difficult to undertake at single-cell level. Furthermore, low cell numbers frequently prohibit direct metabolic assays, for example, in the study of immune cells that are present at tissue sites. In contrast, scRNA-Seq, is broadly accessible and rapidly collected across the human body (Regev et al. 2017), and should allow, in principle, to contextualize metabolic models to the single cell level. A computational method is thus required to systematically address the unique challenges of scRNA-Seq, such as data sparsity, and to capitalize on its opportunities, for example by treating cell populations as natural perturbation systems with a rapidly increasing scale (Svensson, Vento-Tormo, and Teichmann 2018).

Here, Applicants present Compass, an FBA algorithm to characterize and interpret the metabolic heterogeneity among cells, which uses available knowledge of the metabolic network in conjunction with RNA expression of metabolic enzymes. Compass uses single cell transcriptomic profiles to characterize cellular metabolic states at single-cell resolution and with network-wide comprehensiveness. It allows detection of metabolic targets across the entire metabolic network, agnostically of pre-defined metabolic pathway boundaries, and including ancillary pathways that are normally less studied, yet could play an important role in the determining cell function (Puleston, Villa, and Pearce 2017). Applicants applied Compass to Th17 cells, uncovering substantial immunometabolic diversity associated with their inflammatory effector functions. In addition to the expected glycolytic shift, Applicants found diversity in amino acid metabolism, and highlighted a unique and surprising role for the glycolytic reaction catalyzed by phosphoglycerate mutase (PGAM) in promoting an anti-inflammatory phenotype in Th17 cells. Compass is a broadly applicable tool for studying metabolic diversity at the single cell level, and its relationship to the functional diversity between cells.

### Results

### Compass—an Algorithm for Comprehensive Characterization of Single-Cell Metabolism

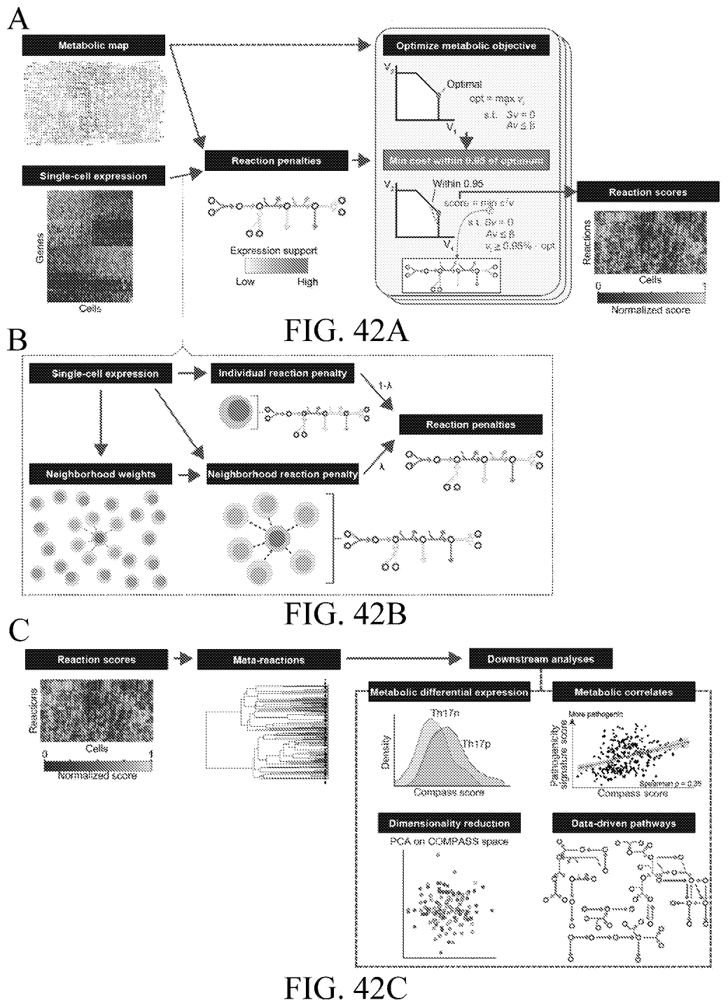

Applicants reasoned that even though the mRNA expression of individual enzymes does not necessarily provide an accurate proxy for their metabolic activity, a global analysis the entire metabolic network (as enabled by RNA-Seq) in the context of a large sample set (as offered by single cell genomics) coupled with strict criteria for hypotheses testing, would provide an effective framework for predicting cellular metabolic status of the cell. This led Applicants to develop the Compass algorithm, which integrates scRNA-Seq profiles with prior knowledge of the metabolic network to infer a metabolic state of the cell (FIG. 42A).

The metabolic network is encoded in a Genome-Scale Metabolic Model (GSMM) that includes reaction stoichiometry, biochemical constraints such as reaction irreversibility and nutrient availability, and gene-enzyme-reaction associations. Here, Applicants use Recon2, which comprises of 7,440 reactions and 2,626 unique metabolites (Thiele et al. 2013). To explore the metabolic capabilities of each cell, Compass solves a series of constraint-based optimization problems (formalized as linear programs) that produce a set of numeric scores, one per reaction (STAR Methods). Intuitively, the score of each reaction in each cell reflects how well adjusted is the cell's overall transcriptome to maintaining high flux through that reaction. Henceforth, Applicants refer to the scores as quantifying the “potential activity” of a metabolic reaction (or “activity” in short when it is clear from the context that Compass predictions are discussed).

Compass belongs to the family of Flux Balance Analysis (FBA) algorithms that model metabolic fluxes, namely the rate by which chemical reactions convert substrates to products and apply constrained optimization methods to find flux distributions that satisfy desired properties (a flux distribution is an assignment of flux value to every reaction in the network) (Orth, Thiele, and Palsson 2010; O'Brien, Monk, and Palsson 2015; Lewis, Nagarajan, and Palsson 2012; Palsson 2015). In the first step, Compass is agnostic to any measurement of gene expression levels and computes, for every metabolic reaction r, the maximal flux vᵣ^{opt} it can carry without imposing any constraints on top of those imposed by stoichiometry and mass balance. Next, Compass relies on the assumption that mRNA expression of an enzyme coding gene should preferably correlate with the flux through the metabolic reaction(s) it catalyzes. It thus assigns every reaction in every cell a penalty inversely proportional to the mRNA expression associated with its enzyme(s) in that cell. Compass then finds a flux distribution which minimizes the overall penalty incurred in any given cell i (summing over all reactions), while maintaining a flux of at least ω·vᵣ^{opt} (here ω=0.95) in r. The Compass score of reaction r in cell i is the negative of that minimal penalty (so that lower scores correspond to lower potential metabolic activity).

Using genome-scale metabolic network allows the entire metabolic transcriptome to impact the computed score for any particular reaction, rather than just the mRNA coding for the enzymes that catalyze it. Applicants reasoned that this helps reduce the effect of instances where mRNA expression does not correlate well with metabolic activity, for example due to post-transcriptional or post-translational modifications. This also mitigates the effects of data sparsity, which is characteristic of scRNA-Seq data. The low transcript signal in scRNA-Seq, which results in the extreme case in false-negative gene detections, magnifies the repercussions of sampling bias and transcription stochasticity, and leads to an overestimation of the variance of lowly expressed genes, which in turn leads in turn to false-positive calling of differentially expressed genes (A. Wagner, Regev, and Yosef 2016). Compass further mitigates data sparsity effects with an information-sharing approach, similar to other scRNA-Seq algorithms (Vallejos, Marioni, and Richardson 2015; Satija et al. 2015; Lun, Bach, and Marioni 2016; Haghverdi et al. 2018; F. Wagner, Yan, and Yanai 2018; Huang et al. 2018; van Dijk et al. 2018; Baran et al. 2019; Grun 2019). Instead of treating each cell in isolation, the score vector for each cell is determined by a combined objective that balances the effects in the cell in question with those in its k-nearest neighbors (based on similarity of their RNA profiles; here, using k=10; FIG. 42B; STAR Methods).

The output of Compass is a quantitative profile for the metabolic state of every cell, which is then subject to downstream analyses (FIG. 42C). These include finding metabolic reactions that are differentially active between cell types or that correlate with continuous properties of cell state (e.g., expression of a certain group of cytokines). It can also provide unsupervised insights into cellular diversity by projecting cells into a low-dimensional space of metabolic activity (e.g., for visual exploration). The statistical power afforded by the large number of individual cells in a typical scRNA-Seq study adds robustness and allows these downstream analyses to gain biological insight despite the high dimension of the metabolic space in which Compass embeds cells. Finally, because Compass does not rely on a predetermined set of metabolic pathways (or gene sets) such as Reactome (Fabregat et al. 2018) or KEGG (Kanehisa et al. 2017), it allows unsupervised derivation of cell-specific metabolic pathways in a data driven way.

Th17 Cell Metabolic Diversity Reflects a Balance Between Glycolysis and Fatty Acid Oxidation, which is Associated with Pathogenicity

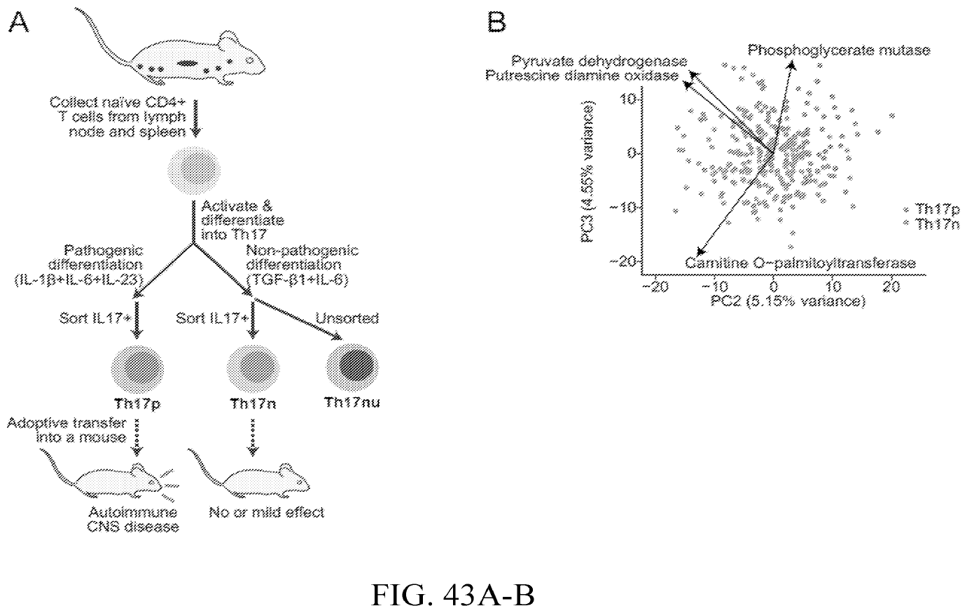

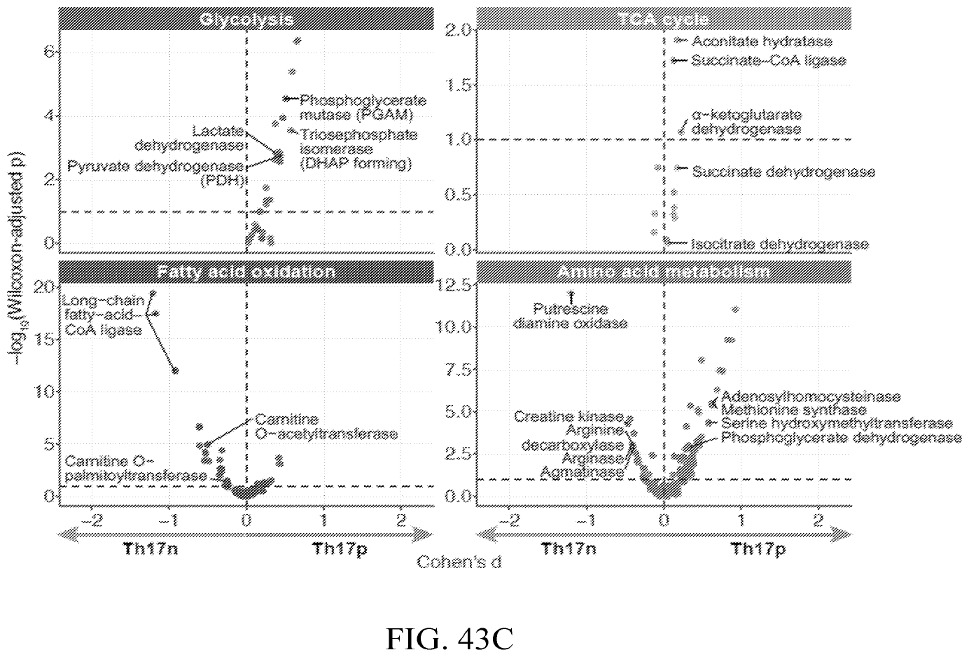

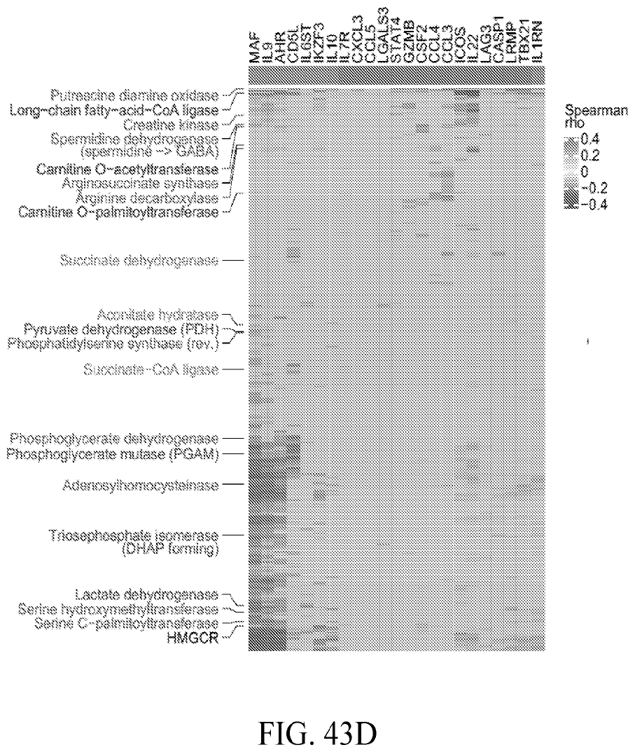

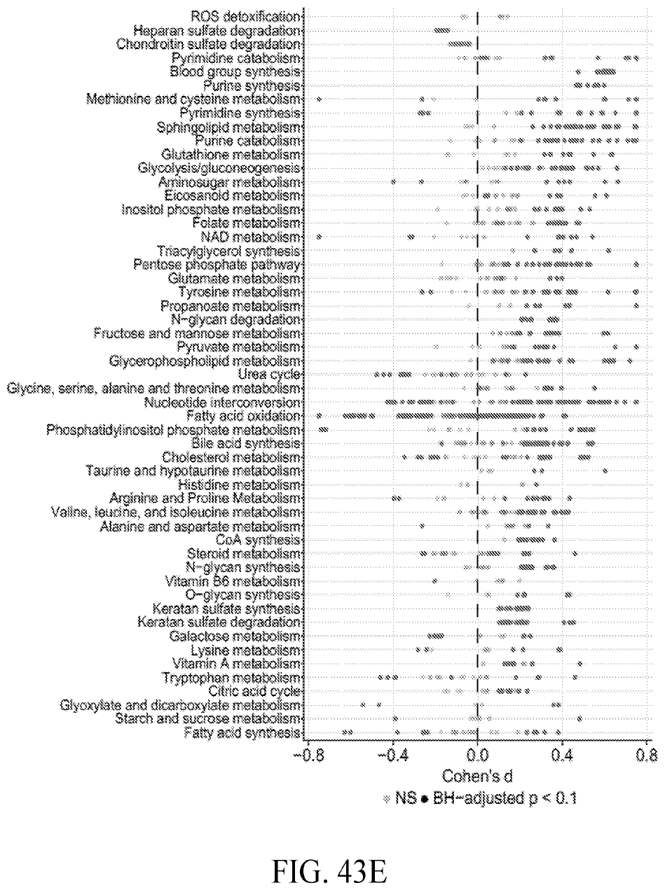

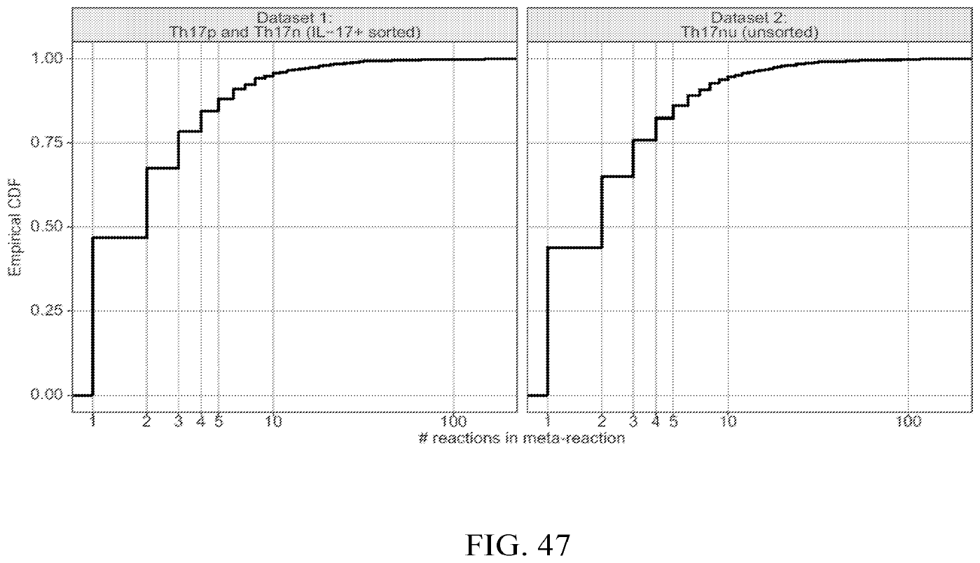

To demonstrate Compass, Applicants applied it to scRNA-Seq data from Th17 cells, differentiated in vitro from naïve CD4+T into two extreme functional states (Ghoreschi et al. 2010; Lee et al. 2012) (FIG. 43A). Differentiation with IL-1β+IL-6+IL-23 creates Th17 cells that upon adoptive transfer into recipient mice induce severe neuroinflammation in the form of experimental autoimmune encephalomyelitis (EAE). Applicants refer to these cells as pathogenic Th17 (Th17p). However, when naïve CD4+ T cells are cultured with TGF-β1+IL-6, the resulting Th17 induce only mild-to-none EAE when adoptively transferred to recipient mice. Applicants refer to these cells as non-pathogenic Th17 (Th17n). Applicants performed a Compass analysis of a dataset Applicants generated in a previous study that included 139 Th17p and 151 Th17n cells sorted for IL-17A/GFP+ and profiled using Fluidigm Cl and SMART-Seq2 (Gaublomme et al. 2015; Wang et al. 2015). Applicants first computed a Compass score for each metabolic reaction in each cell (STAR Methods). Applicants then aggregated reactions that were highly correlated across the entire dataset (Spearman rho>0.98) into meta-reactions (median of two reactions per meta-reaction; FIG. 47) for downstream analysis.

To investigate the main determinants of Th17 cell-to-cell metabolic heterogeneity, Applicants first analyzed the Compass output as a high dimensional representation of the cells which parallels the one produced by scRNA-Seq, but with features corresponding to metabolic meta-reaction rather than transcripts. Applicants performed principal component analysis (PCA) on the meta-reaction matrix, while restricting it to 784 meta-reactions (out of 1,911) associated with core metabolism (STAR Methods) that span conserved and well-studied pathways for generation of ATP and synthesis of key biomolecules.

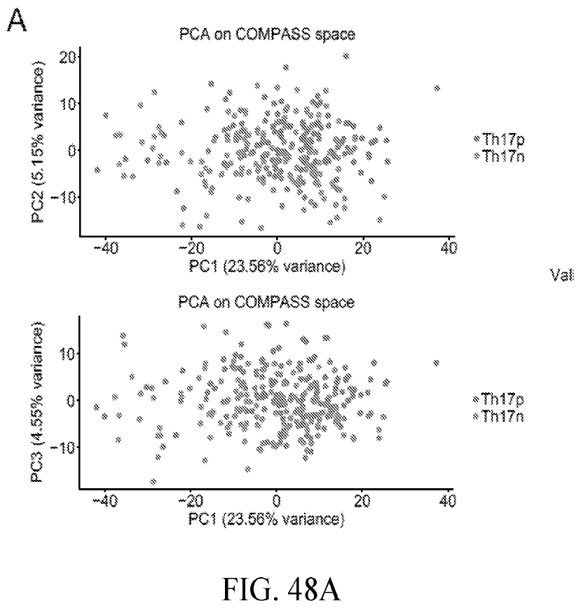

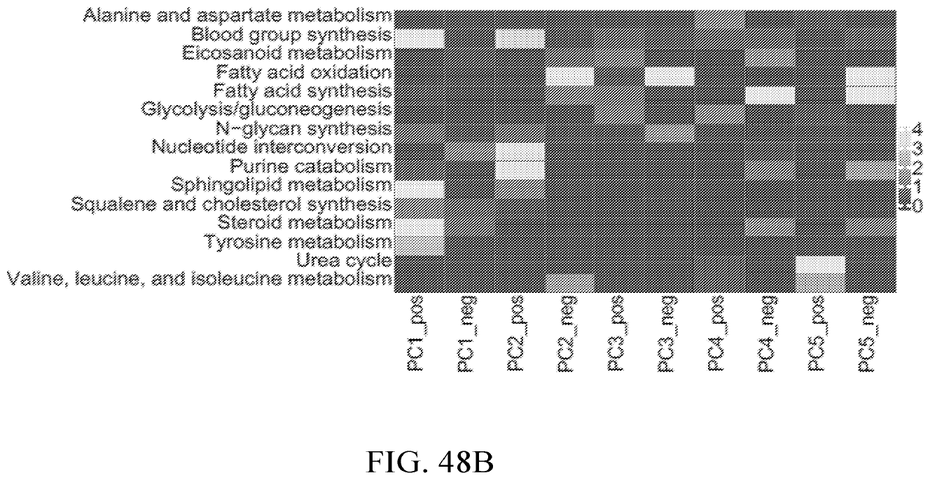

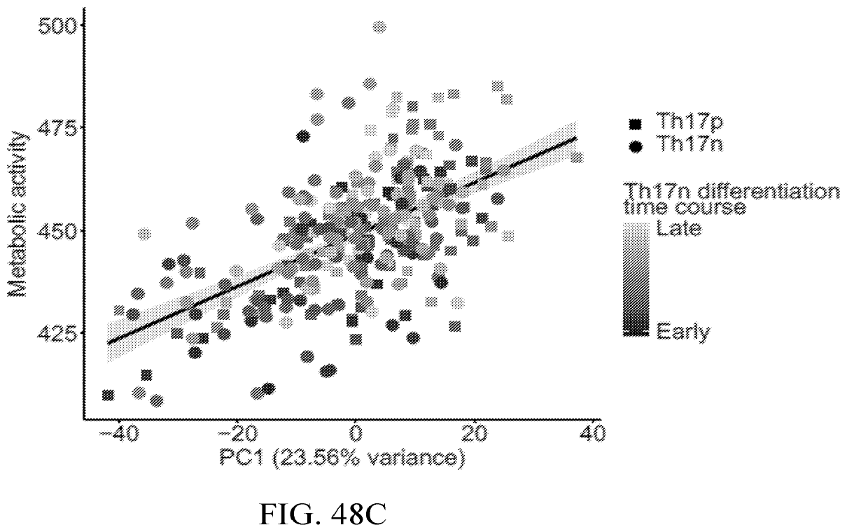

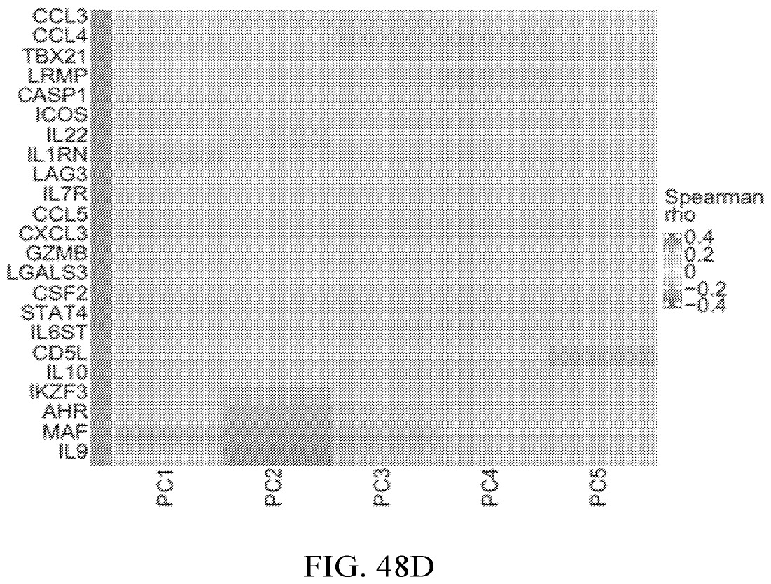

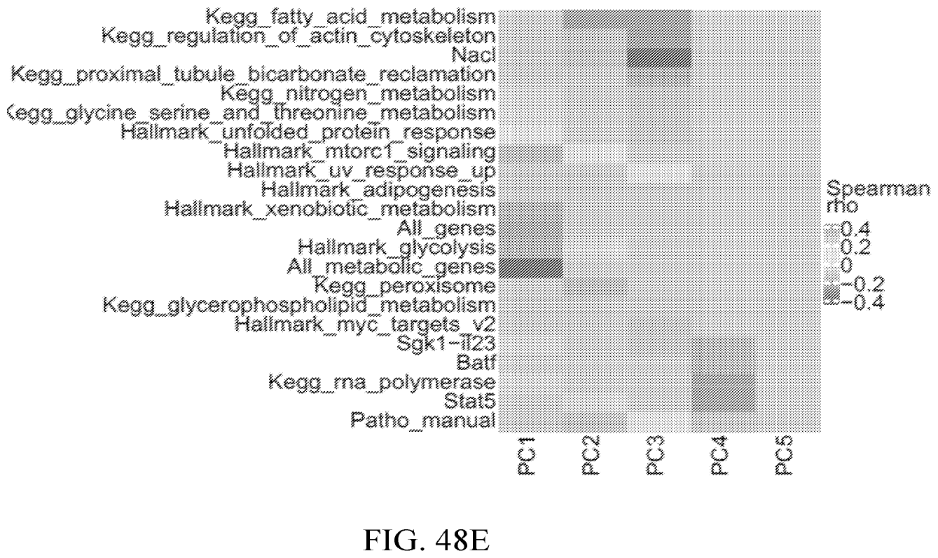

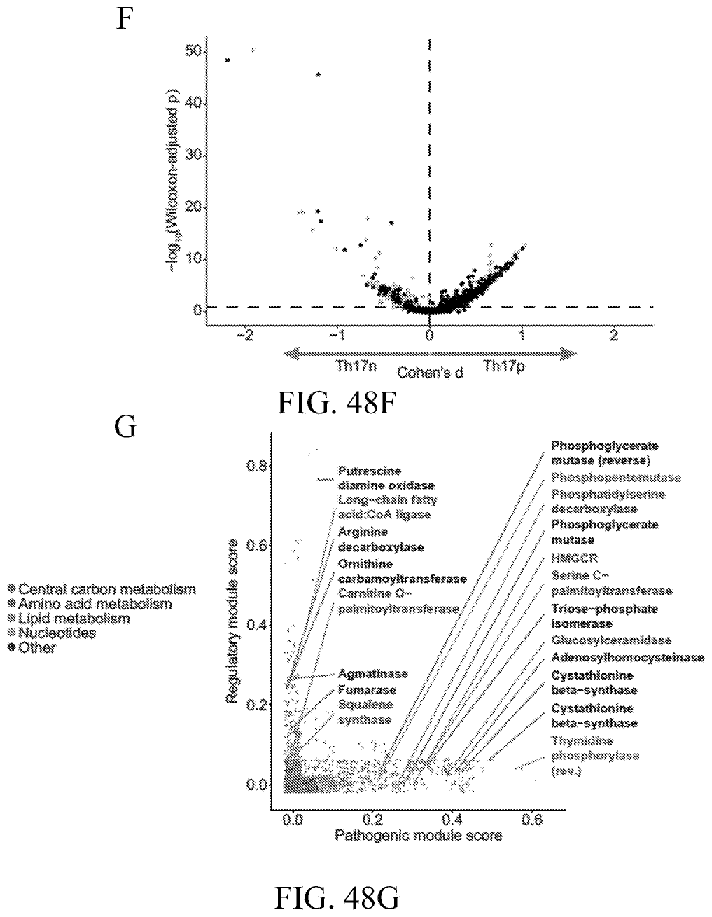

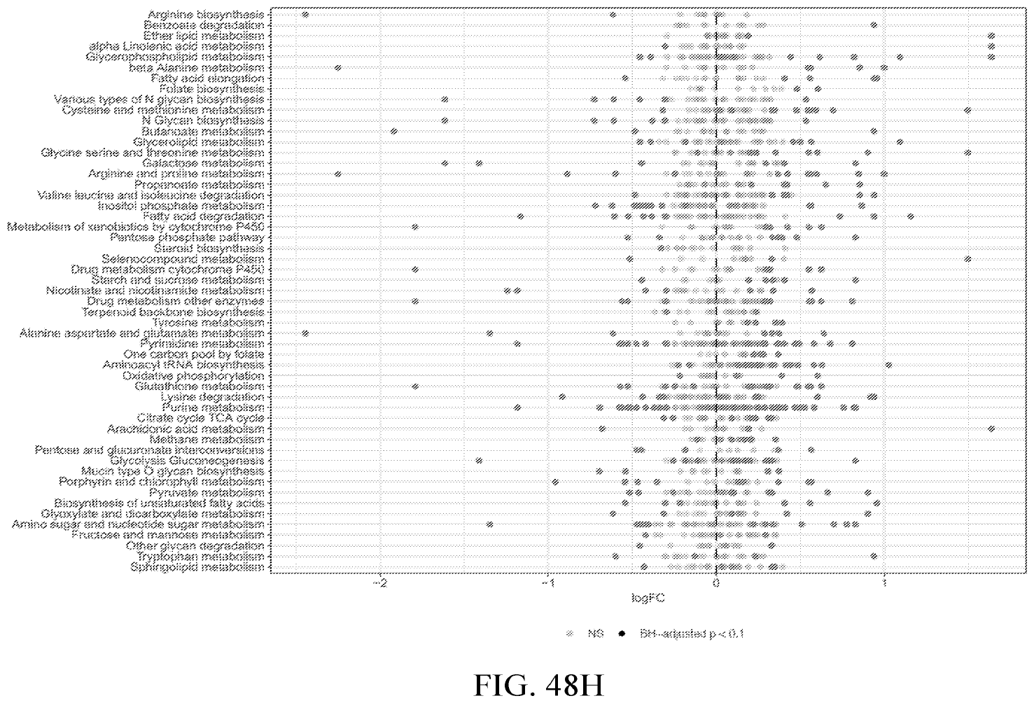

The first two principal components (PCs) of the core metabolism subspace were associated both with overall metabolic activity and T effector functions (FIG. 43B, FIG. 48A,B, Wagner et al., 2020 Table S1). PC1 correlated with the cell's total metabolic activity, defined as the expression ratio of genes coding metabolic enzymes out of the total protein coding genes (Pearson rho=0.36, p<)4*10⁻¹°, as well as a transcriptional signature of late stages of Th17 differentiation over time (Yosef et al. 2013) (FIG. 48C, Pearson rho=0.18, p<0.003) (STAR Methods). PC2 and PC3 represented a choice between ATP generation through aerobic glycolysis versus fatty acid oxidation, which is a prominent finding in immunometabolism while comparing activated Th17 to Tregs, or Teff vs. Tmem cells (Geltink, Kyle, and Pearce 2018). Accordingly, they were correlated with multiple Th17 pathogenicity markers, as well as a signature of Th17 pathogenicity consisting of cytokines, chemokines and transcription factors that are associated with each phenotypic group (Gaublomme et al. 2015; Lee et al. 2012) (FIG. 48D,E). PC2 and PC3 were also noticeably associated with nitrogen metabolism, and were enriched in urea cycle targets whose power to modulate Th17 pathogenicity is demonstrated below and in Example 8. Compass predicts metabolic regulators of Th17 cell pathogenicity

To directly search for metabolic targets that are associated with the pathogenic capacity of individual Th17 cells, Applicants searched for biochemical reactions with differential predicted activity between the Th17p and Th17n conditions according to Wilcoxon's rank sum p value and Cohen's d effect size statistics) and defined pro-pathogenic and pro-regulatory reactions as ones that were significantly different in the Th17p or Th17n direction, respectively (FIG. 43C; FIG. 48F; STAR Methods; Wagner et al., 2020 Table S2). Applicants next discuss several key predictions, which Applicants follow up on in the rest of Example 9 as well as Example 8.

Metabolic reactions in both primary and ancillary pathways were associated with Th17 cell pathogenicity (1,213 or 3,362 reactions out of 6,563 reactions, Benjamini-Hochberg (BH) adjusted Wilcoxon p<0.001 or 0.1, respectively). Many of these reactions are also significantly correlated with the expression of signature genes for Th17 functional activity, which code cytokines and transcription factors (Lee et al. 2012) (FIG. 43D; FIG. 48G; Wagner et al., 2020 Table S3), but not metabolic enzymes. Notably, many classically defined metabolic pathways partially overlapped both with reactions predicted to be pro-pathogenic and reactions predicted to be pro-regulatory (FIG. 43E), highlighting the value in examining single reactions within a global network rather than conducting a pathway-level analysis. A similar result is obtained at the gene expression level—many metabolic pathways included both genes that were upregulated and genes that were downregulated in Th17p compared to Th17n (FIG. 4811, Wagner et al., 2020 Table S4).

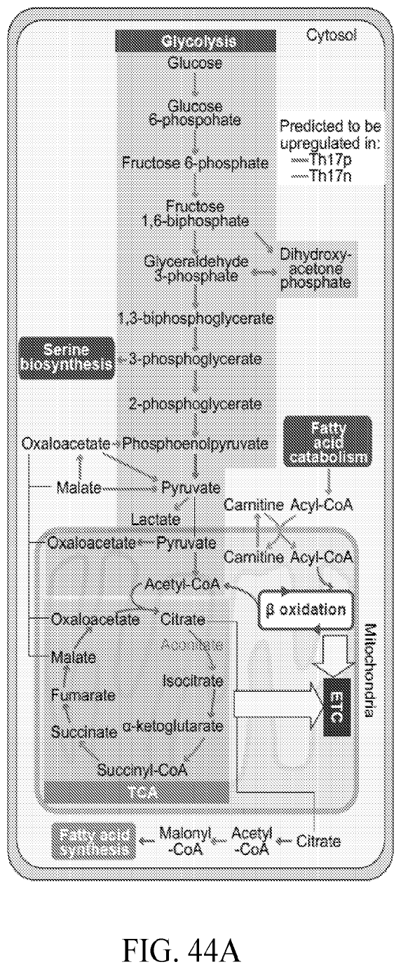

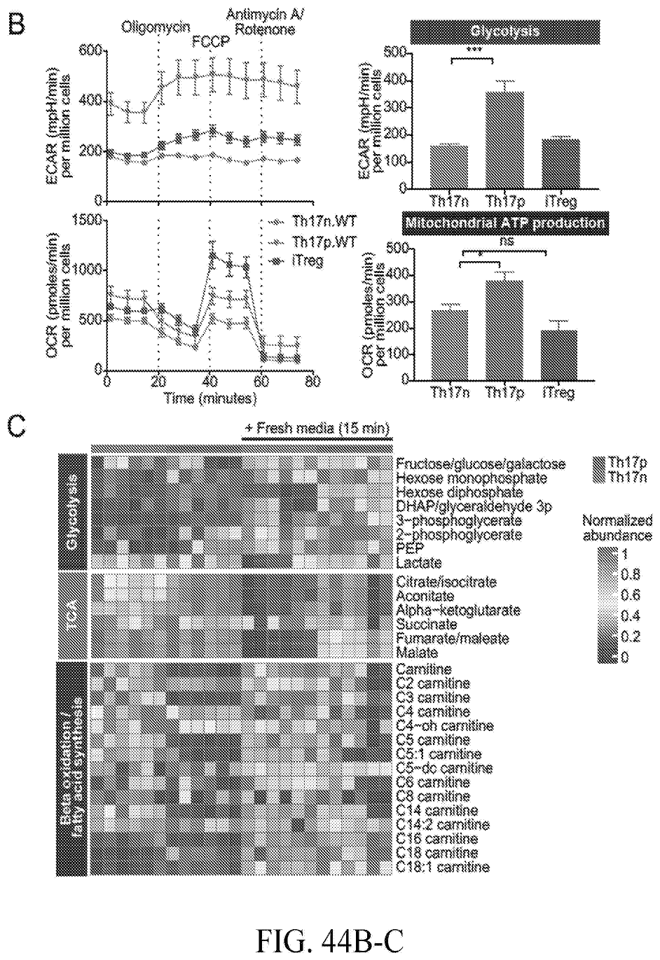

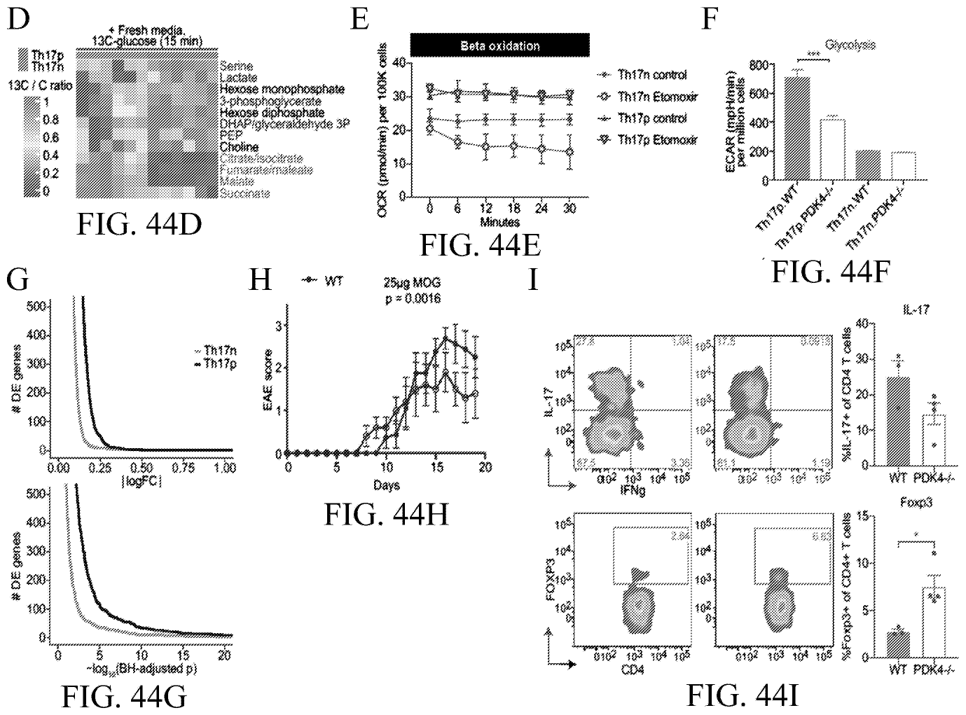

Compass highlighted distinctions in central carbon and fatty acid metabolism between the Th17p and Th17n states, which mirror those found between Th17 and Foxp3+T regulatory (Treg) cells. In central carbon metabolism, Compass predicted that glycolytic reactions, ending with the conversion of pyruvate to lactate are generally more active in the pro-inflammatory Th17p than in the Th17n state (FIGS. 43C and 44A). This parallels previous results showing that Th17 cells upregulate glycolysis even in the presence of oxygen (hence “aerobic glycolysis”), and that interference with this process promotes a Treg fate (L. Z. Shi et al. 2011; Michalek et al. 2011; Gerriets et al. 2015). Compass also predicted an increased activity in Th17p through two segments of the TCA cycle, but bot the cycle as a whole (FIGS. 43C and 44A). A similar breakdown of the TCA cycle in relation with pro-inflammatory function has been shown in macrophages where M1 polarization divided the TCA cycle at the same two points: at isocitrate dehydrogenase (IDH) (Jha et al. 2015), and at succinate dehydrogenase (SDH) (E. L. Mills et al. 2016), which supported their inflammatory functions (L. Shi et al. 2019; E. Mills and O'Neill 2014).

In fatty acid metabolism, Compass predicted that cytosolic acetyl-CoA carboxylase (ACC1), the committed step towards fatty acid synthesis, is upregulated in Th17p, whereas the first two steps of long-chain fatty acid oxidation (long chain fatty acyl-CoA synthetase and carnitine 0-palmitoyltransferase (CPT)) were predicted to be significantly higher in Th17n. These predictions mirror a known metabolic difference between the Th17 and Treg lineages, where Th17 cells rely more on de novo fatty acid synthesis (Berod et al. 2014), whereas Tregs scavenge them from their environment and catabolize them and produce ATP through beta-oxidation (Michalek et al. 2011). Applicants note, however, that recent evidence suggests that CPT may be upregulated in Treg over Th17, but is not functionally indispensable for Treg cells to obtain their effector phenotypes (Raud et al. 2018).

Among ancillary metabolic pathways, Compass highlighted multiple reactions of amino-acid metabolism that are differentially active between Th17p and Th17n cells (FIG. 43C, Wagner et al., 2020 Table S2). It was previously shown that amino acids are important for Th17 cell differentiation (Sundrud et al. 2009), and Compass adds further granularity to these findings. In particular, it predicted that serine biosynthesis from 3-phosphoglycerate, as well as three downstream serine fates—sphingosines, choline, and S-adenosyl-methionine (SAM)—were higher in Th17p. On the other hand, parts of urea cycle and arginine metabolism are significantly associated with both pro-Th17p and pro-Th17n states, (FIG. 43C), suggesting that alternative fluxing within this sub-system may be associated with diverging Th17 cell function. Applicants pursue these predictions and study this subsystem in detail in Example 8. In the following sections Applicants validate the other predictions discussed thus far and build on them to find novel metabolic regulators of Th17 functional states.

### Pathogenic Th17 Cells Maintain Higher Aerobic Glycolysis and TCA Activity, Whereas Non-Pathogenic Th17 Cells Oxidize Fatty Acids to Produce ATP

Applicants validated the Compass prediction that pathogenic and non-pathogenic Th17 functional states differ in their central carbon metabolism (FIG. 44a), using Seahorse assays and liquid-chromatography mass spectrometry (LC/MS) based metabolomics.

First, Applicants compared glycolysis and mitochondrial function of Th17p and Th17n cells. A Seahorse assay (which involves culturing cells with glucose-rich media) confirmed that Th17p cells caused significantly higher extracellular acidification (ECAR) than Th17n, indicating accumulation of lactic acid due to aerobic glycolysis (FIG. 44B, top). Th17p cells also generated significantly more ATP in a mitochondria dependent fashion (FIG. 44B, bottom), consistent with the predicted higher entrance of pyruvate into the TCA cycle despite the diversion of some pyruvate towards the lactate fate.

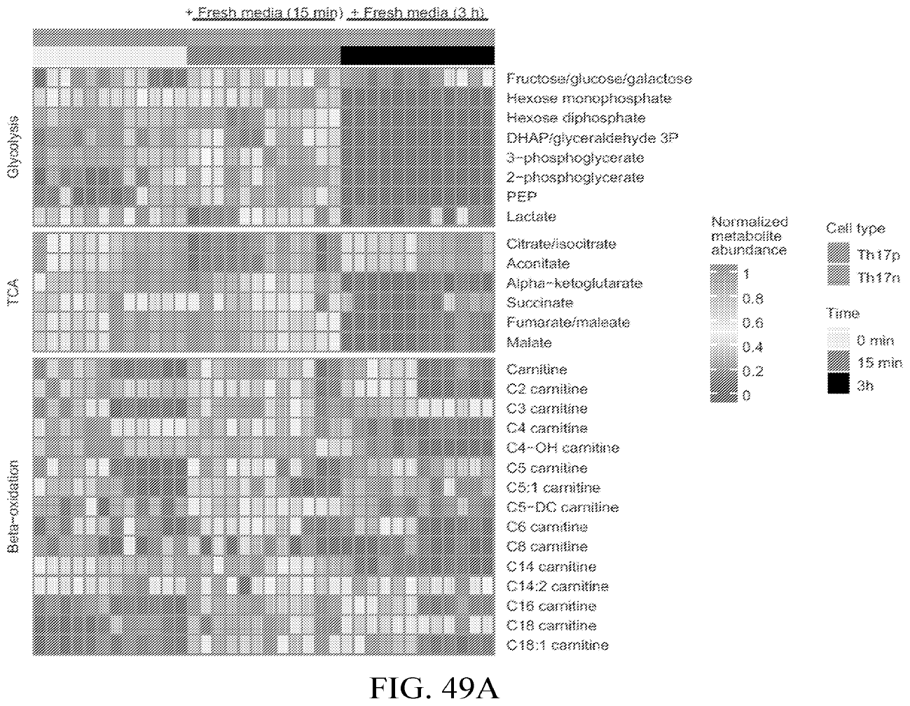

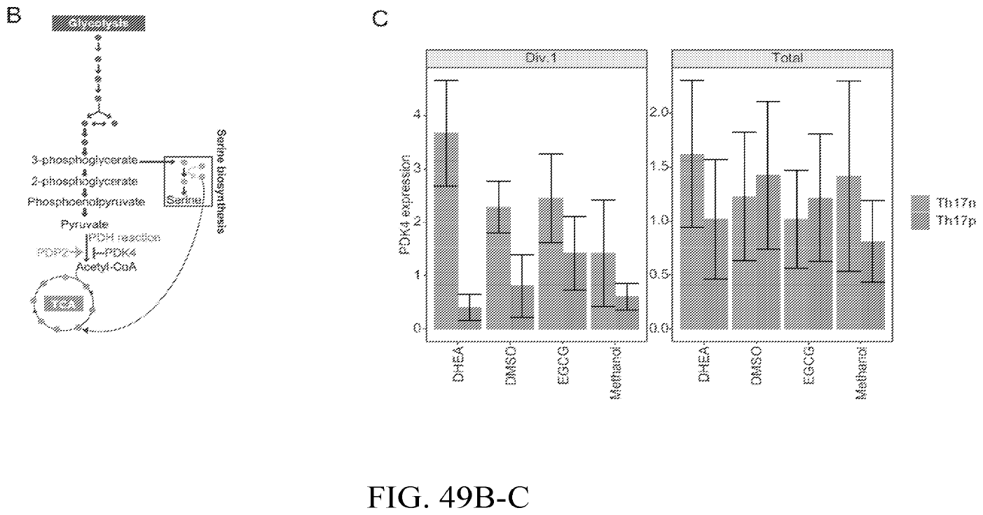

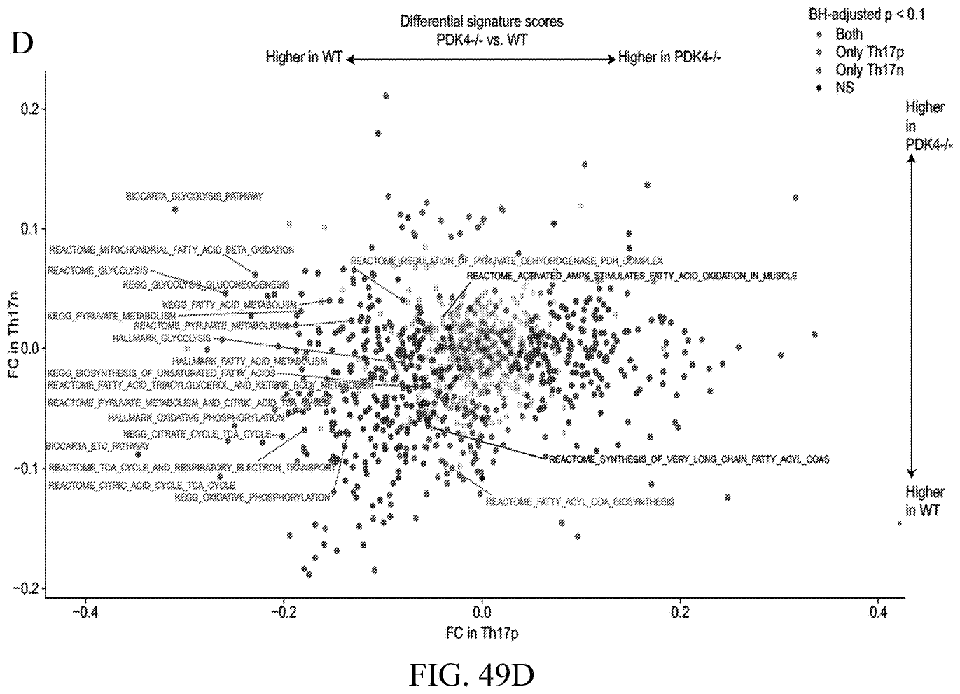

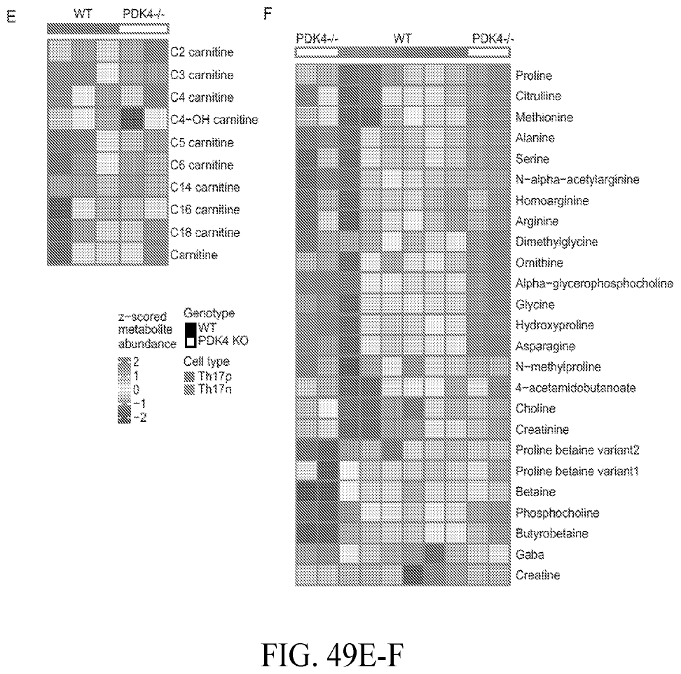

Next, Applicants directly measured metabolites within the glycolysis pathway and TCA cycle using LC/MS based metabolomics. When pulsed with fresh media containing glucose (and rested for 15 minutes), there is a substantial increase in glycolytic metabolites in Th17p but less so in Th17n cells (FIG. 44C, top). Conversely, steady state (pre-pulsing) Th17p and Th17n cells show no apparent difference in glycolytic metabolites, likely due to alternative nutrients or shunting of the glycolytic metabolites into alternative fates. Indeed, after 3 hours with glucose pulsing, the increased level of such metabolites in Th17p return to steady state (FIG. 49).

Interestingly, Compass predicted that two parts of the TCA cycle, but not the cycle as a whole, were upregulated in Th17p: the conversion of citrate to isocitrate and of alpha-ketoglutarate to succinate (mirroring previous findings in macrophages, see above and (Jha et al. 2015; E. L. Mills et al. 2016)). LC/MS Metabolomics analysis of cells at steady state revealed that TCA metabolites were generally more abundant in Th17p than in Th17n, apart from succinate (FIG. 44C, middle). Therefore, both Compass and the metabolomics data point to succinate as a potential metabolic control point.

To test whether not only absolute metabolite levels, but also the relative allocation of carbon into its possible fates differ between Th17p and Th17n cells, Applicants performed a carbon tracing assay with 13C-glucose. Applicants augmented fresh media with 13C-labeled glucose and computed the ratio of the 13C isotope out of the total carbon for each metabolite. Consistent with the predictions, Th17p had significantly higher relative abundance of 13C-labeled glycolytic metabolites than Th17n (FIG. 44D). Furthermore, Th17p preferentially incorporated glucose-derived carbon into serine (which branches from glycolysis; FIG. 49B) and its downstream product choline (FIG. 44D), consistent with shunting of glycolytic metabolites into alternative fates by Th17p cells. This also conforms to the Compass prediction of elevated serine synthesis in Th17p (FIG. 43C). Th17p cells also had significantly lower relative abundance of 13C-labeled TCA metabolites (FIG. 44D), suggesting that the higher level of TCA intermediates observed in Th17p at steady state (FIG. 44C) might not be supported from glucose, but rather from other sources, such as catabolism of amino acids. Taken together, the results suggest that Th17p cells have a higher overall activity through the TCA cycle at steady-state, but quickly switch to aerobic glycolysis when glucose is readily forged from the environment, as observed in the Seahorse, the fresh-media pulsing metabolome assay, and the carbon tracing assay.

Applicants next validated that Th17n cells prefer beta oxidation as predicted by Compass. Metabolomics analysis shows that Th17n cells were enriched in acyl-carnitine metabolites (FIG. 44C, bottom), indicative of active lipid transport through the mitochondrial membrane. This could be a result of either increased lipid biosynthesis or increased catabolism (via beta-oxidation), since acyl-carnitines are intermediates of both processes. However, acyl-carnitines are noticeably more abundant in Th17n, and short- to medium-length acyl groups are particularly more abundant in the steady state and three hours post glucose pulsing (FIG. 49A). This supports the hypothesis that under glucose-poor conditions, Th17n cells, more than Th17p, break fatty acids to produce energy (a process which involves the progressive degradation of long-chain fatty acids into shorter acyl-CoA chains, two carbon atoms at a time). Indeed, when etomoxir was used to block acyl-carnitine transportation across mitochondrial membranes, oxygen consumption rate decreased in Th17n but not Th17p cells, as measured by Seahorse assay (FIG. 44E). Although etomoxir has off-target effects (Raud et al. 2018; Divakaruni et al. 2018), overall the data supports the hypothesis that Th17n cells ultimately divert fatty acid breakdown products into the electron transport chain to generate ATP, which utilizes oxygen as an electron acceptor.

### PDK4-Deficient Th17p Cells Adopt a Non-Pathogenic-Like Central Carbon Program, but Retain a Pathogenic-Like Amino Acid Phenotype

Pyruvate dehydrogenase (PDH) is a critical metabolic juncture through which glycolysis-derived pyruvate enters the TCA cycle (FIG. 49B). Previous studies have shown that the PDH inhibiting kinase 1 (PDK1) is expressed at higher levels in Th17 cells compared to Th1 or FoxP3⁺ Tregs. Consistently, inhibition of PDK1 suppressed Th17 cells but increased the abundance of Tregs (Gerriets et al. 2015), whereas PDH activation by PDH phosphatase catalytic subunit 2 (PDP2) had the opposite effects (Kono et al. 2018). Compass's prediction (FIG. 43D) of increased glycolytic activity, along with these previous studies, prompted Applicants to ask whether PDH has a parallel role in regulating Th17p vs. Th17n states mirroring the reports for Th17 vs. Treg cells (Gerriets et al. 2015). Among PDH inhibitors, PDK4 is of particular interest in immunometabolism, because it plays a role in the cellular starvation response (P. Wu et al. 2000; P. Wu, Peters, and Harris 2001).

To determine whether increased glycolysis, regulated by PDH enzymes, in Th17p cells is important for their global metabolic phenotype, Applicants used PDK4^{-/-} mice for perturbation. Despite the low expression of PDK4 mRNA in Th17 cells (FIG. 49C), Th17 cells differentiated from naïve T cells from PDK4^{-/-} mice had reduced conversion of pyruvate to lactate as measured by ECAR in Th17p but not Th17n conditions (FIG. 44F). This suggests that PDK4-deficiency increases the alternative pyruvate fate, namely entrance into the TCA only in Th17p cells. It also suggests that Th17n cells control pyruvate entrance to the TCA cycle by other means than PDK4.

To further determine the global transcriptional and metabolic changes induced by PDK4 perturbation, Applicants profiled 146 WT and 132 PDK4^{-/-} Th17p cells and 236 WT and 307 PDK4^{-/-} Th17n cells by scRNA-seq using SMART-Seq2. Consistent with the size of the effects on lactate secretion (FIG. 44F), Applicants observed a considerably larger effect of PDK4-deficiency on the transcriptome of Th17p cells (FIG. 44G, Wagner et al., 2020 Table S5). The genes differentially expressed in Th17p cells were primarily enriched in central-carbon metabolism (FIG. 49D and Wagner et al., 2020 Table S6). LC/MS metabolomics showed that PDK4-deficient Th17p cells had notably higher levels of acyl-carnitine, indicating elevated fatty acid transport across mitochondrial membranes (FIG. 49E), similar to WT Th17n cells. Nevertheless, the metabolic phenotype of PDK4-deficient Th17p did not fully shift towards that of Th17n cells. PDK4-deficient Th17p cells retained the WT Th17p metabolome in pathways other than central carbon metabolism, for instance in amino-acid pathways (FIG. 49F). Furthermore, Applicants did not observe significant differences in the expression of key cytokines or transcription factors that are associated with the effector function of these cells.

Seeing that PDK4-deficiency had partially shifted Th17p central carbon metabolism towards the Th17n state in vitro, Applicants next tested the pertaining effects in vivo. To this end, Applicants studied the impact of PDK4− deficiency on the development of EAE, an autoimmune disease induced by pathogenic Th17 cells. Consistent with previous studies that glycolysis promotes inflammation (Gerriets et al. 2015; Gemta et al. 2019; Beckermann, Dudzinski, and Rathmell 2017; Rhoads, Major, and Rathmell 2017), mice with global knockout of PDK4 developed less severe disease as determined by the clinical disease scores (FIG. 44H) with decrease in Th17 cells and increase in the infiltration of Foxp3⁺ Tregs in the CNS of the mice undergoing EAE (FIG. 44I). Applicants therefore conclude that PDK4-deficient Th17p cells resemble Th17n in their central carbon metabolic state, but not in other metabolic pathways. These results prompted Applicants to interrogate metabolic differences outside of central carbon pathways, which are presented in Example 8.

### The Glycolytic Enzyme Phosphoglycerate Mutase (PGAM) Suppresses Th17 Cell Pathogenicity

Thus far, the analysis relied on an inter-population comparison between the extreme states of Th17n and Th17p cells. However, Applicants have previously shown that there is also considerable continuous variation in the transcriptomes of Th17n cells, which spans into pathogenic-like states (Gaublomme et al. 2015). To explore the relationship between metabolic heterogeneity and pathogenic potential within the Th17n subset, Applicants next performed an intra-population analysis of Th17n cells. This also demonstrates that Compass can be applied to scRNA-Seq data in cases where the states of interest (e.g., Th17n vs. Th17p) are either unknown or cannot be experimentally partitioned into discrete types. To perform an intra-population Compass analysis of single Th17n cells, Applicants correlated the Compass scores associated with each reaction with the pathogenicity gene signature scores of the respective cells (STAR Methods).

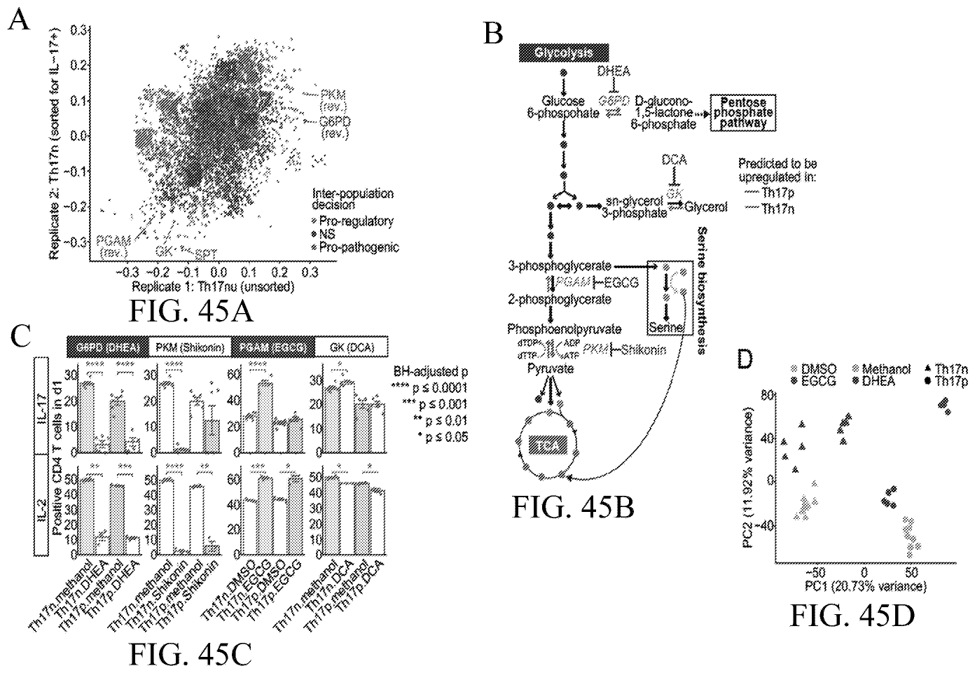

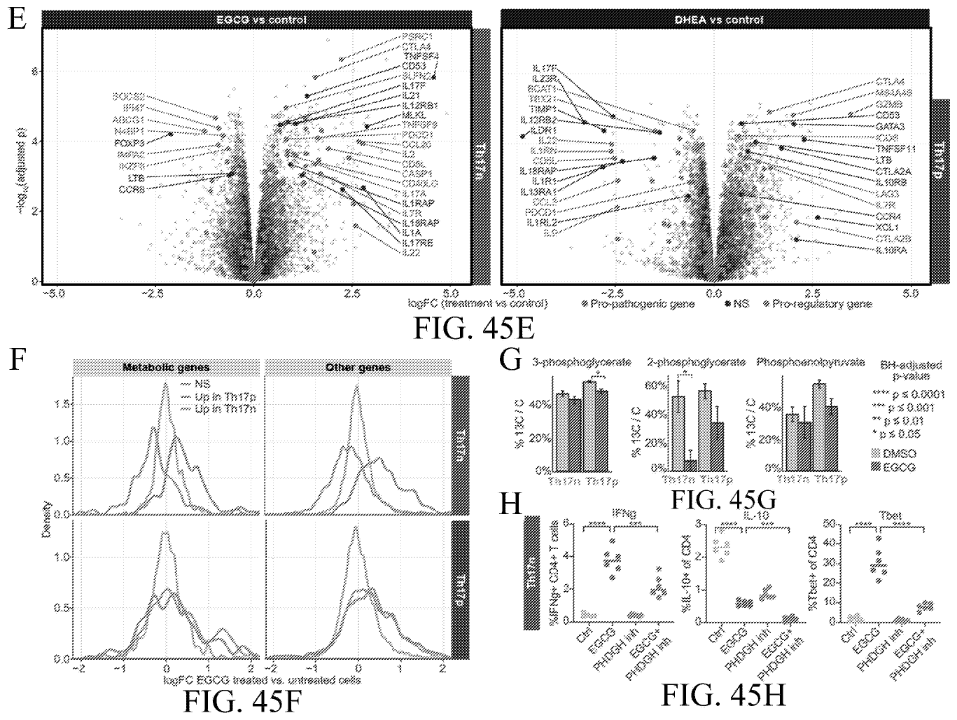

While the resulting correlations of individual reactions with the pathogenicity score were largely consistent with the results of the inter-population analysis (Th17p vs. Th17n) (FIG. 45A, Wagner et al., 2020 Tables S7-8), the intra-population analysis predicted that some glycolytic reactions may be negatively, rather than positively (as in the inter-population analysis), associated with Th17 pathogenicity. The most notable of these reactions was the one catalyzed by the enzyme phosphoglycerate mutase (PGAM), which was negatively associated with pathogenicity in the intra-population analysis of Th17n cells (FIG. 45A), but positively associated with Th17p cells in the inter-population analysis (FIG. 43B,C). This prediction was unexpected because increased glycolysis is generally understood to support pro-inflammatory phenotypes in Th17 cells (Discussion).

To functionally validate the glycolytic targets associated with Th17 cell pathogenicity by the intra-population analysis, Applicants used chemical inhibitors against enzymes driving the top two glycolytic reactions that were most positively correlated (regulated by pyruvate kinase muscle isozyme [PKM], and glucose-6-phosphate dehydrogenase [G6PD]) and top two that were most negatively correlated (phosphoglycerate mutase [PGAM], and glucokinase [GK]) with the pathogenicity score (FIG. 45B). The inhibitors were shikonin (inhibits PKM2), dehydroepiandrosterone (DHEA, inhibits G6PD), epigallocatechin-3-gallate (EGCG, inhibits PGAM1), and 2,3-dihydroxypropyl-dichloroacetate (DCA, inhibits GK) (STAR Methods).

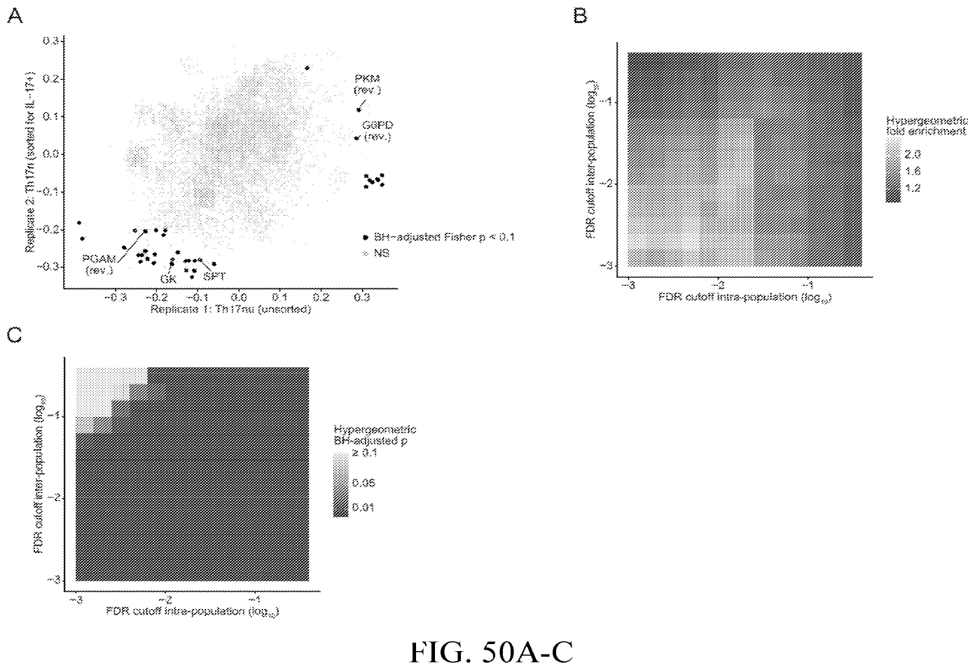

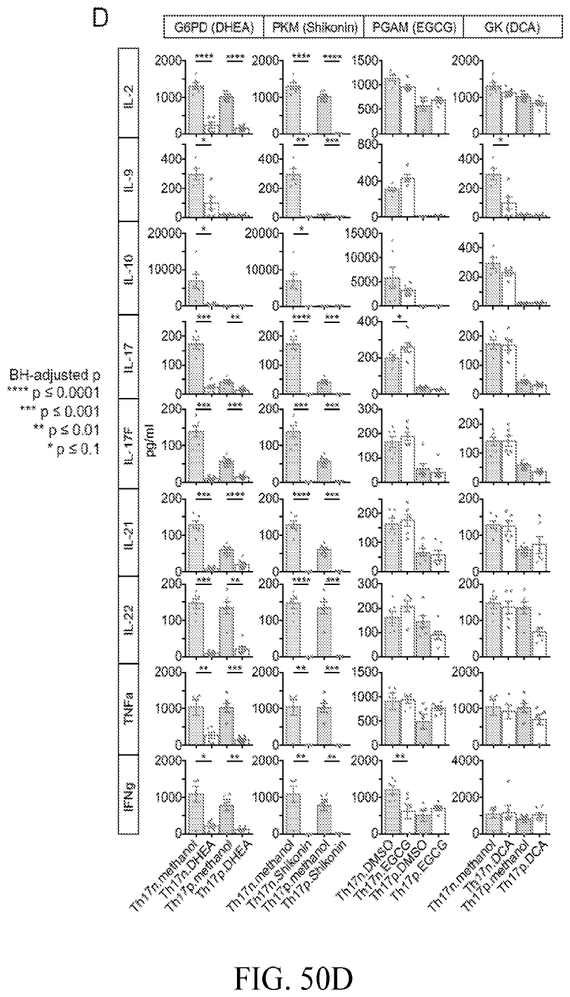

Applicants first analyzed the effects of inhibitors on Th17n and Th17p cell differentiation and function using flow cytometry (FIG. 45C). Due to the possibly deleterious effects of blocking these central reactions on cell viability, Applicants used the highest dose of each inhibitor that did not affect cell viability (compared to solvent alone). Applicants further used flow cytometry to restrict the analysis to cells that had undergone one division (dl) so as to exclude arrested cells or cells that have been blocked from activation and expansion. In addition, since two different solvents (DMSO and methanol) were needed for different inhibitors, every treatment group was matched with an appropriate vehicle control. Applicants found that IL-17 expression conformed to the prediction made by Compass. It was significantly upregulated by chemical inhibition of the two enzymes (PGAM or GK) predicted to suppress pathogenicity, and downregulated by chemical inhibition of the two enzymes (G6PD or PKM) predicted to promote pathogenicity (FIG. 45C). This was further confirmed when profiling a larger set of cytokines secreted by Th17 cells: inhibition of PKM or G6PD curtailed all cytokine production suggesting that these enzymes are important for overall T effector functions. In contrast, cells with PGAM or GK inhibition, at the optimal concentration, mostly retained their cytokine profile with a few exceptions (FIG. 50).

To analyze the impact of perturbing glycolytic enzymes on the transcriptome, Applicants used bulk RNA-Seq to profile Th17n and Th17p cells grown in the presence of either the predicted pro-regulatory inhibitor DHEA (inhibiting G6PD) or the predicted pro-inflammatory inhibitor EGCG (inhibiting PGAM) (FIG. 45D-F). The first principal component (PC1), which is the main axis of variation in the data, represented as expected, the pathogenicity phenotype. In both Th17n and Th17p cells, EGCG shifted cell profiles towards a more pathogenic state on PC1, whereas DHEA shifted them to a less pathogenic state (FIG. 45D). The difference between the two vehicle controls was inconsequential compared to cell type and interventions.

To better interpret the drug-induced transcriptional changes, Applicants examined individual genes whose expression is associated with either Th17n or Th17p effector function as wells as global transcriptomic shifts (FIG. 45E,F and Wagner et al., 2020 Table S9). A comparison of DHEA to vehicle control identified a large number of effector genes that are modulated (FIG. 45E, right). These include a significant decrease in IL23R and TBX21 transcripts in both Th17p and Th17n, two genes critical for Th17 cell pathogenicity, and in IL9 and IL1RN, two genes highly expressed in non-pathogenic Th17 cells (Lee et al. 2012). Conversely, EGCG clearly strengthened the pathogenic transcriptional program in Th17n, globally upregulating pro-inflammatory genes (e.g., IL22, IL7R, and CASP1) and (to a more limited extent) downregulating pro-regulatory ones (e.g., IKZF3) (FIG. 45E, left and 45F). The global shift towards the pro-inflammatory Th17 program was observed both in metabolic and non-metabolic transcripts, supporting the hypothesis that PGAM inhibition by EGCG effected a network-wide metabolic shift that mediated emergence of a pro-inflammatory Th17 program (FIG. 45F).

To verify that the effect of EGCG was mediated by a specific inhibition of PGAM (rather than an off-target effect) Applicants conducted a carbon tracing assay in which the cell's medium was supplemented with 13C-glucose (STAR Methods). PGAM inhibition with EGCG led to a sharp decrease (from 51% 13C ratio to 7% in Th17n and from 55% to 33% in Th17p) in 13C contents of 2PG (PGAM's product) but not 3PG (PGAM's substrate) or any other glycolytic metabolite that Applicants were able to measure (FIG. 45G). Interestingly, 13C ratio of PEP (one step downstream of 2PG) was not changed as well. This suggests that the effect of the inhibitor is restricted (at least within glycolysis) to the PGAM reaction that lies directly downstream of 3PG.

As the serine biosynthesis pathway is more active in Th17p than in Th17n (FIG. 43C) and lies directly downstream of 3PG (FIG. 45B), Applicants asked whether inhibiting serine biosynthesis can rescue the effect of PGAM inhibition. To this end, Applicants treated Th17n cells with inhibitors to PGAM and PHGDH (phosphoglycerate dehydrogenase), alone or in combination. Applicants found that further inhibiting PHDGH rescued the upregulation of Tbet and IFNg induced by EGCG but not its impact on IL-10 suppression (FIG. 4511).

Taken together, an intra-population Compass analysis predicted that within the Th17n compartment, the glycolytic PGAM reaction inhibits, rather than promotes, pathogenicity. This prediction relied on heterogeneity within the Th17n population, yielding results that are contrary to those from inter-population comparisons of Th17 to Treg or Th17p to Th17n. EGCG specifically inhibited this reaction, and promoted a transcriptional state indicative of a more pro-inflammatory potential, as evidenced by a global shift in the transcriptome toward a Th17p-like profile. RNA-Seq further supported the hypothesis that EGCG mediates its effects by altering the cellular metabolic profile.

### PGAM Inhibition Exacerbates, Whereas G6PD Inhibition Ameliorates, Th17-Mediated Neuroinflammation In Vivo

To test the functional relevance of the transcriptome shifts induced by EGCG and DHEA in vivo, Applicants used the adoptive T cell transfer system, so that the effect of inhibitors is limited to T cells rather than all cells in the host. Applicants generated Th17n and Th17p cells from naive CD4+ T cells isolated from 2D2 TCR-transgenic mice, with specificity for MOG 35-55, and transferred them into wildtype mice to induce EAE.

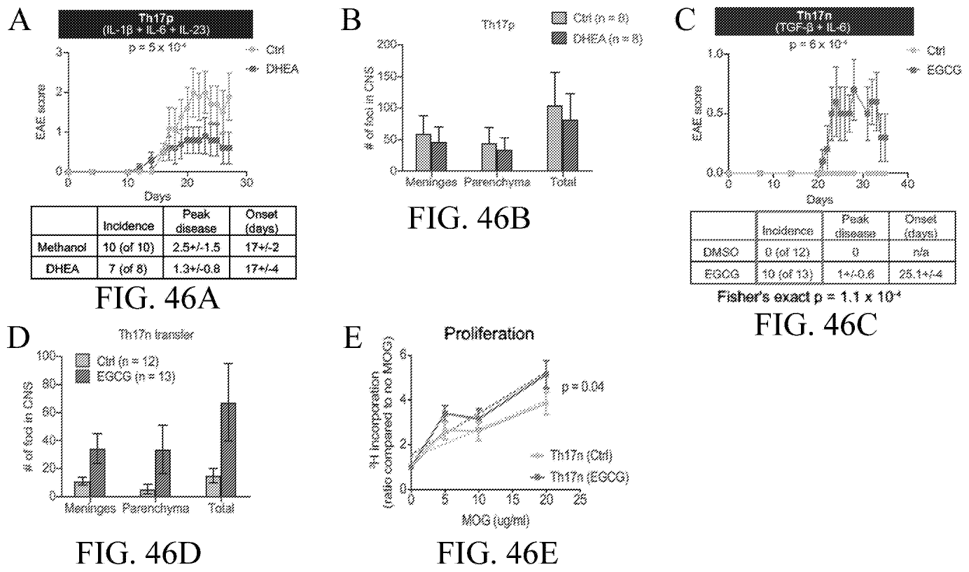

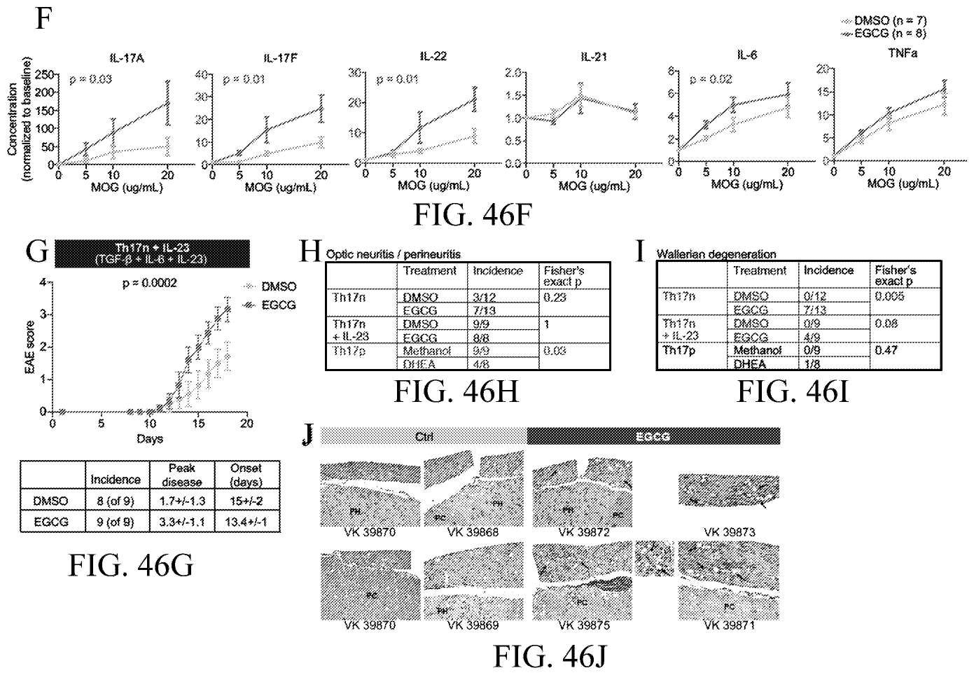

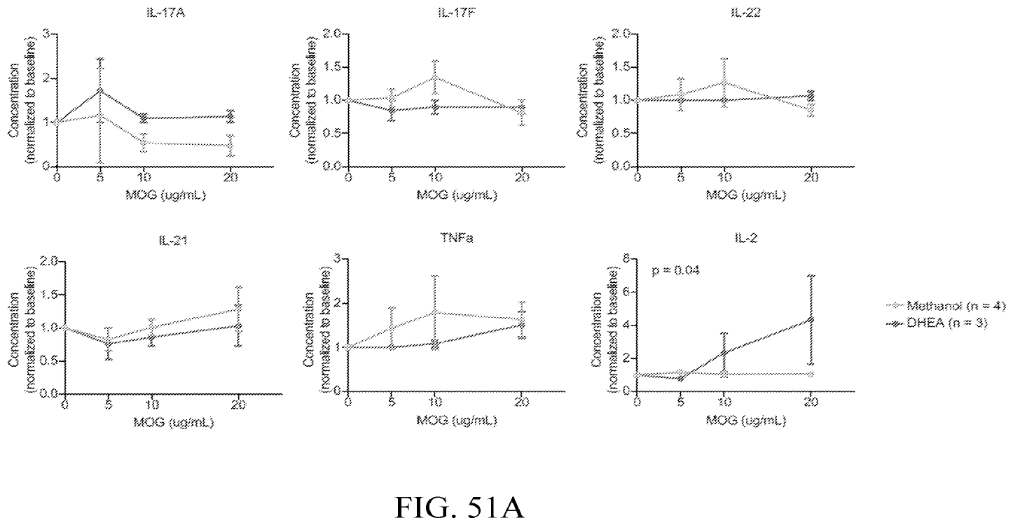

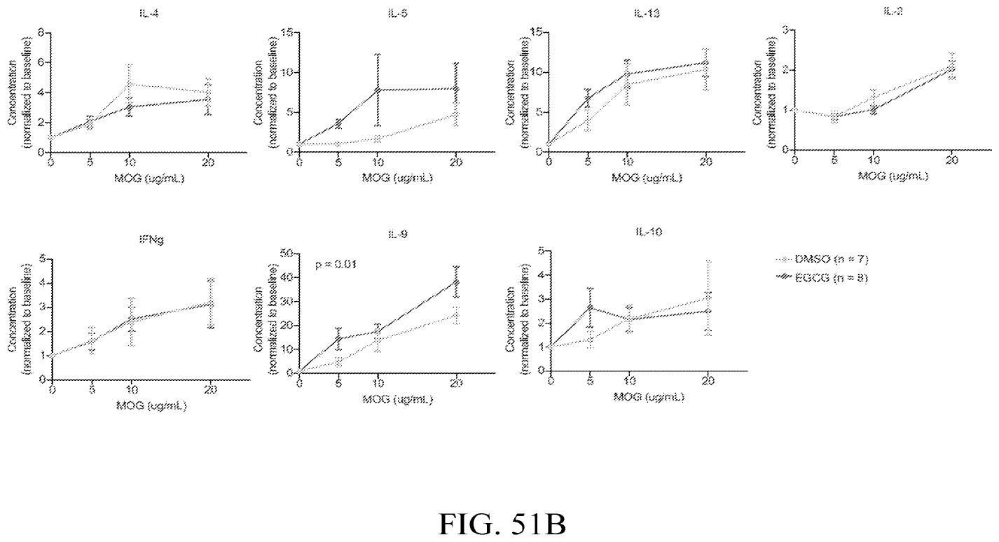

Consistent with Compass prediction, Th17p cells treated with DHEA reduced the severity of disease at peak of EAE in the recipients (FIG. 46A). By the time the mice were sacrificed, however, the number of lesions in CNS was not significantly different (FIG. 46B), and surviving mice showed no significant alterations in antigen-specific cytokine secretion in response to MOG, except for increased IL-2 in the DHEA treated group (FIG. 51A). More interestingly, and in agreement with Compass predictions, EGCG-treated Th17n cells induced EAE, albeit in mild form, whereas solvent treated cells failed to produce any consequential neuroinflammation (FIG. 46C). Recipients of EGCG-treated Th17n cells had a significantly higher EAE incidence rate (10/12) compared with the control group (0/12, Fisher's exact p=1.1*10⁻⁴). Consistent with the clinical disease, histological analyses revealed an increased number of CNS lesions in in both the meninges and the parenchyma of mice that were injected with EGCG-treated Th17 cells (FIG. 46D). While there was only a small difference in antigen-specific T cell proliferation (FIG. 46E), there was a significant increase in secretion of IL-17, IL-17F, IL-22 and IL-6 (FIG. 46F and FIG. 51B) in response to antigen in cells isolated from draining lymph node of mice transferred with EGCG-treated Th17n cells. As EGCG treated non-pathogenic Th17n cells induced only mild EAE, Applicants asked whether EGCG will further enhance encephalitogenicity of Th17 cells if IL-23 is included in the differentiation cultures, which stabilizes the Th17 phenotype (Awasthi et al. 2009; McGeachy et al. 2009; Zhou et al. 2007; Aggarwal et al. 2003). IL-23− treatment indeed enhanced EAE disease severity, but still Th17n cells treated with IL-23+EGCG induced significantly more severe EAE than their IL-23+solvent-treated counterparts (FIG. 46G). Histopathology across all experiments revealed that EGCG treatment of Th17n cells promoted, whereas DHEA treatment Th17p cells restricted, optic neuritis/perineuritis in host mice (FIG. 4611). Interestingly, mice transferred with EGCG-treated Th17 cells (Th17n or Th17n with IL-23) were the only experimental group to produce Wallerian degeneration in proximal spinal nerve roots (FIG. 46I, J).

In conclusion, Compass correctly predicted metabolic targets including glycolytic pathways whose deletion affected Th17 function. Importantly, it was able to pinpoint a glycolytic reaction that suppresses Th17 pathogenicity, which runs contrary to the current understanding that aerobic glycolysis as a whole is associated with a pro-inflammatory phenotype in Th17 cells.

### Discussion

Applicants presented Compass—a flux balance analysis (FBA) algorithm for the study of metabolic heterogeneity among cells based on single-cell transcriptome profiles and validated a number of predictions by metabolome and functional analyses. Compass successfully predicted metabolic targets in both central and ancillary pathways based on its network approach. These results support the power of transcriptomic-based FBA to make valid predictions in a mammalian system.

Glycolysis is a central regulator of T cell function. Compass predicted an association between aerobic glycolysis and Th17 pathogenicity, which accords with multiple previous results tying elevated glycolysis with T cell inflammatory functions. However, a Compass-based data-driven analysis based on scRNAseq unexpectedly revealed that not all glycolytic reactions promote the pro-inflammatory phenotype in Th17 cells. This result was obtained via an intra-population analysis of individual cells. It serves as a further example to the power of studying single-cell heterogeneity within seemingly homogenous populations (here, Th17n), which allowed Applicants to identify a novel regulator that would have otherwise been missed at a population level (here, a comparison of Th17p and Th17n). This result further demonstrates that despite the common assumption that glycolysis promotes inflammatory functions in Th17 cells and other immune compartments (E. L. Pearce et al. 2013; E. J. Pearce and Everts 2015; MacIver, Michalek, and Rathmell 2013; O'Neill and Pearce 2016), a more nuanced view is in order (Van den Bossche, O'Neill, and Menon 2017; Newton, Priyadharshini, and Turka 2016).

Static FBA algorithms assume that the system under consideration operates in chemical steady state (Varma and Palsson 1994). Even under this assumption, there remains an infinite number of feasible flux distributions that satisfy the preset biochemical constraints. Therefore, most studies assume that the system (here, a cell) aims to optimize some metabolic function, usually production of biomass or ATP (Damiani et al. 2019). However, whereas such objectives may successfully predict phenotypes of a unicellular organism (Lewis et al. 2010), they are ill-suited for studying mammalian cells (Adler et al. 2019). To overcome this challenge, rather than optimizing a single metabolic objective function, Compass optimizes a set of objective functions, each estimating the degree to which a cell's transcriptome supports carrying the maximal theoretical flux through a given reaction. The result is a high dimensional representation of the cell's metabolic potential (one number per reaction). A biological signal (e.g., differences in reaction potential) can be detected in this high-dimension owing to the statistical power afforded by the large number of cells in a typical scRNA-Seq dataset. Nonetheless, there is no inherent limitation preventing one from applying Compass to study bulk (i.e., non-single-cell) transcriptomic data.

The database of metabolic reactions Applicants used pertains to human cells, and as such the study does not address differences between human and mouse metabolism. In addition, the database provides a global view of the metabolic capabilities of a human cell, accrued from various sources and in diverse cell types. Not all reactions may be functional in a studied cell type, or under particular physiological conditions. This concern can be addressed to some extent by procedures for deriving organ-specific metabolic models (Opdam et al. 2017). Moreover, the metabolic state of a cell depends on the nutrients available in its environment, which are often poorly characterized. Here, the computations assume an environment rich with nutrients, which accords with the studied in vitro growth media. Modifying this to better represent physiological conditions should increase the algorithm's predictive capabilities, especially for cells derived in vivo, where nutrient scarcity may be a limiting factor, and nutrient availability may vary between tissues.

One of the outstanding challenges in the field of single cell genomics is translating the vast data sets presented in cell atlases into an actionable knowledge resource, i.e. using observed cell states to deduce molecular mechanisms and targets (Tanay and Regev 2017). Compass was designed with this challenge in mind, and addresses it in the metabolic cellular subsystem, which can be tractably modeled in silico. In light of the wide appreciation of cellular metabolism as a critical regulator of physiological processes in health and disease, Applicants expect Compass to be useful in predicting cell metabolic states, as well as actionable metabolic targets, in diverse physiological and pathologic contexts.

### Example 9 STAR Methods

### 1 Experimental Procedures

### 1.1 T Cell Differentiation Culture

Naive CD4+CD44-CD62L+CD25− T cells were sorted using BD FACSAria sorter and activated with plate-bound anti-CD3 and anti-CD28 antibodies (both at 1 mg/ml) in the presence of cytokines at a concentration of 0.5×10⁶ cells/ml. For Th17 differentiation: 2 ng/ml of rhTGFb1, 25 ng/ml rmIL-6, 20 ng/ml rmIL-1b (all from Miltenyi Biotec) and 20 ng/ml rmIL-23 (R & D systems) were used at various combinations as specified in figures. For differentiation experiments, cells were harvested at 68 hours for RNA analysis and 72-96h for flow cytometry analysis and Seahorse assay.

Seahorse assay was performed and seahorse media was prepared following manufacturer instructions (Agilent). Cells were re-stimulated with PMA/ionomycin for four hours in the presence of brefaldin and monensin before analysis for cytokines by intracellular cytokine staining. Cytokine concentrations in supernatants of in vitro cultures were analyzed by the LegendPlex Mouse Th Cytokine Panel (13-plex) (BioLegend) according to the manufacturer's instructions and analyzed on a FACS LSR II (BD Biosciences).

### 1.2 Smart-Seq Single-Cell RNA Sequencing

Full experimental details are given in (Gaublomme et al., 2015). Briefly, Applicants sequenced CD4+ naive T cells 48 hrs post polarization under one of these conditions, ultimately retaining after quality tests 130 Th17n cells unsorted for IL-17 (denoted Th17nu in the present manuscript), 151 IL-17A/GFP+Th17n cells, and 139 IL-17A/GFP+Th17p cells. Unlike (Gaublomme et al., 2015), in the present study Applicants analyzed the unsorted and sorted cells independently from one another. The sorted cells (Th17n and Th17p) were used for the inter-population analysis, and the unsorted cells (Th17nu) were used for the intra-population analysis.

1.3 Estimation of Transcript Abundance from RNA Se-Quenching

Applicants aligned single-cell SMART-Seq libraries with Bowtie2, quantified TPM gene expression with RSEM, and performed QC as Applicants described in detail in a previous publication (Fletcher et al., 2017). This computational pipeline is a massively revised and updated version of the one originally used to analyze these libraries (Gaublomme et al., 2015). Batch effects and other nuisance factors were normalized with a model chosen empirically with SCONE (Cole et al., 2019). Bulk RNA-Seq were processed with a modified variant of the same pipeline.

### 1.4 Differential Gene Expression

For the Smart-Seq libraries, due the absence of UMIs in the dataset, differentially expressed genes were called through a linear model _tted to TPM values with the limma R package and with a mean-variance trend added to the empirical Bayes prior (Ritchie et al., 2015). For the bulk RNA libraries, differentially expressed genes were called with limma-trend or limma-voom (Law et al., 2014) depending on the variance of library sizes, as recommended in the limma package manual (Smyth, 2019).

### 1.5 Inhibitors

All chemical inhibitors were purchased from Sigma with the exception of EGCG (Selleck Chemicals) and tested in a wide range of dose (20 nM-200 uM) on Th17 cells. The lowest dose that resulted in minimal impact on cell viability is used for functional evaluation: EGCG, the inhibitor for PGAM1 (Li et al., 2017), was used at 20-50 uM; DHEA, inhibitor for G6PD (Schwartz and Pashko, 2004), was used at 50 uM; DCA, inhibitor for GK (Westergaard et al., 1998, Tisdale and Threadgill, 1984), was used at 40 uM; Shikonin, inhibitor for PKM2 (Zhao et al., 2018, Chen et al., 2011), was used at 10 uM; and PKUMDL-WQ-2101, inhibitor for PHGDH (Wang et al., 2017), was used at 12.5 uM.

### 1.6 Mice

C57BL/6 wildtype (WT) and PDK4−/− mice were obtained from Jackson Laboratory (Bar Harbor, Me.). WT 2D2 transgenic mice were bred in house. All experiments were performed in accordance to the guidelines outlined by the Harvard Medical Area Standing Committee on Animals at the Harvard Medical School (Boston, Mass.).

### 1.7 Experimental Autoimmune Encephalomyelitis (EAE)

For adoptive transfer EAE, naive T cells (CD4+CD44-CD62L+CD25-) were isolated from 2D2 TCR-transgenic mice and activated with anti-CD3 (1 mg/ml) and anti-CD28 (1 mg/ml) in the presence of differentiation cytokines for 68h. Cells were rested for 2 days and restimulated with plate-bound anti-CD3 (0.5 mg/ml for pathogenic condition; 1 mg/ml for non-pathogenic condition) and anti-CD28 (1 mg/ml) for 2 days prior to transfer. Equal number (2 to 8 million) cells were transferred per mouse intravenously. EAE is scored as previously published (Jager et al., 2009).

### 1.8 LC/MS Metabolomics and Carbon Tracing

### 1.8.1 Assays

For untargeted metabolomics, Th17 cells were differentiated as described. Culture media were snap frozen. Cells were harvested at 96h. 1×10⁶ cells per sample were snap frozen and extracted in either 80% methanol (for fatty acids and oxylipids) or isopropanol (for polar and nonpolar lipids). Two liquid chromatography tandem mass spectrometry (LC-MS) methods were used to measure fatty acids and lipids in cell extracts.

For carbon tracing experiments Th17 cells were differentiated as described. Thereafter, cells were washed and cultured in media supplemented with 8 mM [U-13C]-glucose for 15 min or 3 hrs.

### 1.8.2 Statistical Analysis

Differentially abundant metabolites were found with Student's t-test and a significance threshold of BH-adjusted p<0:1.

To find metabolites with differential 13C relative abundance, Applicants computed the ratio y_{i,j} of 13C out of the total carbon contents for each metabolite i in sample j. Let |Cᵢ| be the number of carbon atoms in metabolite i, and let x_{c,i,j} be the measured signal of metabolite i in sample j (subsequent to all normalization and QC procedures) in which there are exactly c 13C atoms. Applicants define the 13C/C ratio:

$${y}_{i , j} = \frac{{\sum }_{t = 0}^{\vert {C}_{i} \vert} t \cdot {x}_{t , i , j}}{\vert {C}_{i} \vert \cdot {\sum }_{t = 0}^{\vert {C}_{i} \vert} {x}_{t , i , j}}$$

### 2 Downstream Analysis of Compass Scores

### 2.1 Core Metabolic Reactions and Meta-Reactions

In this application, Applicants define Applicants core metabolism based on reaction metadata included in the Recon2 database. Recon2 assigns a confidence score to each reaction based on the level of evidence supporting it between 1 (no evidence) and 4 (biochemical evidence), with 0 denoting reactions whose confidence was not evaluated. Since pathways generally considered part of primary metabolism are also the best studied ones, Applicants define a reaction as belonging to core metabolism if (a) its Recon2 confidence is either 0 or 4; and (b) it is annotated with an EC (Enzyme Commission) number. Applicants chose to label reactions with unevaluated confidence (i.e., Recon2 confidence score of 0) as part of core metabolism because some of them were found to be key reactions in primary metabolic pathways based on manual correction. The definition of core metabolism is equivalent to taking the set of all metabolic reactions in Recon2, but excluding reactions that either don't have an annotated EC number or for which the Recon2 curators explicitly specified they do not have direct biochemical support. Applicants define a meta-reaction as belonging to core metabolism if it contains at least one core reaction. Metabolic genes are defined as the set of genes annotated in Recond2 (Section 4.7)

2.2 Inter-Population Analysis: Finding Reactions with Differential Potential Activity

To test for differential potential-activity of reactions based on Compass predictions, Applicants computed for each meta-reaction M the Wilcoxon's rank sum between the Compass scores of M in the two populations of interest (here, Th17p and Th17n). Effect size were further assessed with Cohen's d statistic, defined as the difference between the sample means over the pooled sample standard deviation. Let n₁, x₁, s₁be the number of observations in population 1, and the sample mean and standard deviation of their scores in a given meta-reaction, respectively (with a similar notation for population 2). Then with

$$d = \frac{( \overline{{x}_{1}} - \overline{{x}_{2}} )}{s}$$

$$s = \sqrt{\frac{( {n}_{1} - 1 ) {s}_{1}^{2} + ( {n}_{2} - 1 ) {s}_{2}^{2}}{{n}_{1} + {n}_{2} - 2} )} .$$

The resulting p values are adjusted with the Benjamini-Hochberg (BH) method. Note that so far, the computation was done for meta-reactions. Applicants assigned all reactions r∈M the Cohen's d and Wilcoxon's p value that were computed forts. Applicants call a reaction differentially active if its adjusted p is smaller than 0:1. The computation was done on all reactions in the network (namely, both core and non-core reactions).

### 2.3 Transcriptomic Signatures

### 2.3.1 Th17 Pathogenicity and Other T Cell State Signatures

Applicants used a transcriptomic signature that Applicants have previously shown to capture a Th17 cell's pathogenic capacity (Gaublomme et al., 2015, Wang et al., 2015). Briefly, for each cell compute the average z-scored expression (log(1+TPM)) of pro-pathogenic markers (CASP1, CCL3, CCL4, CCL5, CSF2, CXCL3, GZMB, ICOS, IL22, IL7R, LAG3, LGALS3, LRMP, STAT4, TBX21) and pro-regulatory markers (AHR, IKZF3, IL10, IL1RN, IL6ST, IL9, MAF), with the latter group multiplied by −1.

### 2.3.2 a Compendium of T Cell State Signatures

A compendium of T cell state transcriptomic signatures was described in (Gaublomme et al., 2015). Every signature consists of two gene subsets: a set of positively associated genes and an optionally empty set of negatively associated genes. A scalar signature value is computed for every cell based on its transcriptome profile as described above for pathogenicity. Signatures that are based on KEGG (Kanehisa et al., 2017) pathways or similar resources are constructed by defining the set of positively-associated genes as the ones belonging to the pathway and defining the set of negatively-associated genes as an empty set.

### 2.3.3 Total Metabolic Activity of a Cell

Applicants defined the total metabolic activity of a cell as the sum expression of metabolic enzyme coding genes over the sum expression of all protein coding genes in log-scale TPM (transcripts per million) units. Applicants computed the partial correlation between this quantity and cell PC1 coordinates, while controlling for the sum expression of all protein coding genes in the cells (the aforementioned divisor) to verify the correlation does not arise from the ratio of protein-coding to non-protein coding RNA in the RNA libraries. The correlation was more significant when not controlling for the covariate (Pearson rho=0:56, p<3·10⁻¹⁶).

### 2.3.4 Late-Stage Th17 Differentiation

Applicants defined a transcriptomic signature for late-stage differentiation of Th17 cells based on microarray data from (Yosef et al., 2013). Applicants assigned microarrays into three differentiation stages as described in that paper into early (up to 4h), intermediate (6-16h) and late (20-72h) and fitted with the limma R package a linear model for the discrete 3-level stage covariate. Applicants called differentially expressed genes (BH-adjusted p<0:05 and log 2 fold-change ≥3) and used them to define a transcriptomic signature as described above.

computed genes differentially expressed between the late

2.4 Intra-Population Analysis: Correlation with a Quantitative Cellular Trait

Here, the population was Th17n cells, in one of two biological replicates, and the quantitative trait was a transcriptomic pathogenicity signature. For every meta-reaction M, Applicants computed Spearman correlation of its Compass scores and the pathogenicity scores across all cells. Applicants assigned all reactions r∈M the Spearman correlation and its statistical significance that were computed for M Note that the division of reactions to meta-reactions is dataset-specific and therefore a reaction can belong to different meta-reactions in each of the replicates. So far, the computation was done independently for the two biological replicates. Applicants then computed for each reaction r the Fisher combined p value of the two p values corresponding to the statistical significance of its Spearman correlation with the pathogenicity scores in the two replicates. The combined Fisher p values were adjusted with the Benjamini-Hochberg (BH) method. Applicants call a reaction significantly correlated (or anti-correlated) with the pathogenicity score if its adjusted Fisher combined p is smaller than 0.1. The search space was limited to core reactions.

### 2.5 Manual Curation of Central Carbon Predictions

Applicants manually curated the significant predictions of the central carbon metabolism pathways discussed in the manuscript (glycolysis, TCA cycle, and fatty acid synthesis/oxidation). Recon2 takes account of metabolite localization, and reactions may be functional in more than one cellular compartment. For every reaction, Applicants picked the prediction corresponding to the pertinent cellular compartment (here, cytosol or mitochondria, as shown in FIG. 3a). Note that Compass operates independently on the forward and backward directions of every reaction, and that the direction is denoted in the pathway diagrams of this manuscript.

### 3 Running Compass

### 3.1 Software

Compass is available at github.com/YosefLab/Compass The algorithm is highly parallelizable. It currently supports execution on multiple threads in a single machine, submission to a Torque queue, and execution on a single machine on Amazon Web Services (AWS). The current implementation relies on the IBM ILOG CPLEX Optimization Studio, which is free for academic use.

### 3.2 Gene Expression Input

The main input is gene expression matrix G in which rows correspond to genes and columns to RNA libraries. Applicants assume that G is (i) already normalized to remove batch and other nuisance effects; (ii) scaled to CPMs or TPMs. In the present application Applicants used TPMs; and (iii) in linear (i.e., not log) scale.

### 3.3 Running Compass on Bulk (i.e., Non-Single-Cell) Inputs

The current manuscript presents the algorithm in the context of single cells, where Compass leverages the statistical power afforded by the large number of observations (cells). Nevertheless, there is no inherent limitation preventing one from applying Compass to study bulk (i.e., non-single-cell) transcriptomic data. In this case, Applicants recommend disabling the information-sharing feature by setting lambda=0 in Algorithm 2. There is also no limitation preventing one from applying Compass to non-RNA-Seq transcriptomic data, such as microarrays.

### 3.4 Scalability

For prohibitively large datasets, the number of cells (observations) can be reduced by partitioning the cells into small clusters and treating the average of each cluster as an observation in downstream analysis. Two implementations of this approach are micropools (DeTomaso et al., 2019), implemented in the VISION R package (github.com/YosefLab/VISION), and meta-cells (Baran et al., 2019) (tanaylab.github.io/metacell). No pooling was necessary for the analysis presented in this manuscript (i.e., the results are on a single cell level). If cell clusters are large enough, one may choose to skip the information-sharing procedure, which is equivalent to setting the parameter λ=0 in Algorithm 2.

In addition, the number of reactions in the GSMM can be reduced as well by not executing Algorithm 2 on blocked reactions (Section 4.5), non-core reactions (Section 2.1), or reactions outside a predetermined set of metabolic pathways that are of interest. Applicants note that Applicants do not suggest excluding non-blocked reactions from the network altogether (which would result in neglecting their effects on reactions that are of interest), but rather only excluding them from the R(G) matrices in Algorithm 2.

### 4 the Compass Algorithm in Detail

### 4.1 Metabolic Network

Applicants Used the Recon2 GSMM (Thiele et al., 2013), which Applicants Transformed into a unidirectional network by replacing bidirectional reactions with the respective pair of unidirectional reactions. Consequently, ux values are always nonnegative.

### 4.2 Metabolic Genes

Throughout this application, metabolic genes are defined as the set of genes annotated in Recon2. Note that Compass uses only the expression of metabolic genes and ignores other transcripts.

### 4.3 in Silico Growth Medium

The results of flux balance analysis significantly depend on the nutrients made available to the GSMM, referred to as the in silico growth medium. Since exact medium composition is mostly unknown even for common in vitro protocols and in vivo models, Applicants chose a rich in silico medium where all nutrients for which a transporter exits are made available in an unlimiting quantity.

### 4.4 Notation

In the following sections Applicants denote:
n: number of cells (or RNA libraries).
m: number of metabolic reactions in the GSMM.
C: the set of cells in the data. (C=n).
R: the set of metabolic reactions in the GSMM. (R=m).
rev(r): the reverse unidirectional reaction of reaction r, which has the same stoichiometry but proceeds in the opposite direction. g: number of genes in a given transcriptome dataset.
S: the stoichiometric matrix defined in the GSMM, where rows represent metabolites, columns represent reactions, and entries are stoichiometrical coefficients for the reactions comprising the metabolic network. Reactions for uptake and secretion of a metabolite are encoded as having only a coefficient of 1 and −1 in the metabolite's row entry, respectively, and 0 otherwise.
   For a matrix M=(m_{i,j}) and a function ƒ: [symbol] → [symbol] Applicants use ƒ(M) to denote (where the intention is obvious from the context) the respective point-wise transformation, namely ƒ(M):=(ƒ(m_{i,j})).

### 4.5 Transcriptome-Agnostic Preparatory Step

For a given GSMM (here, Recon2), Applicants run once a preparatory step that does not depend on transcriptome data and cache the results (Algorithm 1).

Algorithm 1: Find maximal reaction fluxes

| | input: GSMM | |
|---|---|---|
| | output: maximal flux vᵣ^{opt} that every reaction r can carry | |
| 1 | foreach r ∈ [symbol] do | |
| 2 | \| | ${v}_{r}^{opt} := {maximize}_{v ∈ {ℝ}^{m}} {v}_{r}$ |
| | \| | s.t. |
| | \| | (i) S · v = 0 |
| | \| | (ii) α ≤ v ≤ β |
| | \| | (iii) vᵣₑᵥ(r) = 0 |

| | |
|---|---|
| 3 | end |

Constraint (i) constrains the system to steady state (Varma and Palsson 1994). Constraint (ii) is interpreted as ∀i: αᵢ≤vᵢ≤βᵢ and encodes directionality and capacity limits for reactions, including uptake and secretion limits. Constraint (iii) ensures that when evaluating the maximum ux for each reaction, its reverse reaction carries no flux to avoid the creation of a futile cycle. This does not prevent futile cycles longer than 2 edges, which can be avoided only by more time-consuming computations (Schellenberger, Lewis, and Palsson 2011).

Note that the GSMM may contain blocked reactions vᵣ^{opt}=0 that can be excluded from the next steps to speed the computation.

4.6 from Gene Expression to Reaction Expression

By reaction expression, Applicants denote a matrix {R(G)}_{m×n} that is conceptually similar to the gene expression matrix {G}_{g×n}. The columns are the same RNA libraries (e.g., cells) as in G, but rows represent single metabolic reactions rather than transcripts. An entry R_{r,j} in the matrix R(G) is a quantitative proxy for the activity of reaction r in cell j. Applicants omit the dependence on gene expression matrix and denote simply R when G is obvious from the context.

The reaction expression matrix is created by using the Boolean gene-to-reaction mapping included in the GSMM, similar to the approach taken by (Becker and Palsson, 2008, Shlomi et al., 2008). Let G={x_{i,j}} and consider a particular reaction r in a particular cell j. If a single gene with linear-scale expression x is associated with r, then the reaction's expression will be R_{r,j}=log₂(x+1). If no genes are associated with r then R_{r,j}=0.

If the reaction is associated with more than one gene, then this association is expressed as a Boolean relationship. For example, two genes which encode different subunits of a reaction's enzyme are associated using an AND relationship as both are required to be expressed for the reaction to be catalyzed. Alternately, if multiple enzymes can catalyze a reaction, the genes involved in each will be associated via an OR relationship. For reactions associated with multiple genes in this manner, the Boolean expression is evaluated by taking the sum or the mean of linear-scale expression values x when genes are associated via an OR or AND relationship, respectively. This way, the full gene(s)-to-reaction associations is evaluated to arrive at a single summary expression value for each reaction in the GSMM.

### 4.7 Information Sharing Between Single Cells (Smoothing)

To mitigate the sparseness and stochasticity of single-cell measurements, Compass allows for a degree of information-sharing between cells with similar transcriptional profiles. Given a gene expression G, Applicants compute k-nearest neighbors (kNN) graph based Euclidean distances in reduced dimension, obtained by taking the top 20 principal components of G. The PCA is computed over all the genes in G, not only metabolic ones.

Let R(C)={r_{i,j}} and

$${w}_{i , j} = \{ \begin{matrix}\frac{1}{k} , if cell j is in the k \text{-} nearest \text{-} neighborhood of cell i , \\ 0 , otherwise\end{matrix}$$

then R^{N} (G)={r_{i,j}^{N}} where

$${r}_{i , j}^{N} = {\sum }_{c ∈ 𝒞} {w}_{j , c} {r}_{i , c}$$

### 4.8 Main Algorithm

Compass transforms a gene expression matrix {G}_{g×n}, where rows represent genes and columns represent RNA libraries (usually, single cells, although bulk RNA can also be used as discussed below) into a matrix {C}_{m×n} of scores where rows represent metabolic reactions, columns are the same RNA libraries as in the gene expression, and an entry quantifies a proxy for potential reaction activity. More precisely, the entry quantifies the propensity of the cell to use that reaction.

The algorithm is summarized in (Algorithm 2). First, Applicants convert the gene expression matrix G_{g×n} into a reaction expression matrix R_{m×n} which is parallel to the gene expression matrix, but with rows representing single metabolic reactions rather than transcripts. Applicants convert R into a penalty matrix P_{m×n} by point-wise inversion. Whereas R represents gene expression support that a reaction is functional in the cell, P represents the lack thereof (which will be used in a linear program below). The computation of R and P occurs also for the neighborhood of each cell for to smooth results and mitigate single-cell technical noise. Then, Applicants solve a linear program for every reaction r in every cell i to find the minimal resistance of cell i to carry maximal flux through r. Last, Applicants scale the scores, which also entails negating them such that that larger scores will represent larger potential activities (instead of larger penalties, hence smaller potential activity). The final scores indicative of a cell's propensity to use a certain reaction. Applicants interpret it as a proxy for the potential activity of the reaction in that cell.

In step 10 of Algorithm 2, a high penalty yr, indicates that cell c is unlikely, judged by transcriptomic evidence, to use reaction r. Cells whose transcriptome are overall more aligned with an ability to carry ux through a reaction will be assigned a lower penalty y_{r,c}. With regards to the correctness of the step, recall that the GSMM is unidirectional and therefore ∀i. vᵢ>0.

### 4.9 Meta-Reactions

Rows in the Craw matrix that correspond to reactions that are topologically close in the metabolic network can be highly correlated. Applicants therefore hierarchically cluster Craw rows by Spearman distance. Applicants call the resulting clusters meta-reactions and each represents a set of closely correlated metabolic reactions. Note that the division into meta-reactions is data-driven and does not rely on canonical metabolic pathway definitions. Therefore, the division is dataset-dependent—for example, two reactions might be closely correlated and clustered in the same meta-reaction in one cell type, but not in another.

After computing the hierarchical clusters over rows of C^{raw}, Applicants merged leaves in which Spearman similarity (namely 1−ρ, with ρ being the Spearman correlation) by averaging the respective rows. In the present application, Applicants used ρ=0.98. Applicants denote the row-merged matrix {C^{meta-raw}}m′×n

### 4.10 Scaling Raw Compass Scores

By definition, all entries in C^{meta-raw} are non-negative. Applicants scale it in Algorithm 3 (the min in the second step denotes matrix-wide minimal entry)

Algorithm 2: Compass

| | |
|---|---|
| input: | Gene expression matrix {G}_{g×n} |
| | GSMM |
| | Pre-computed maximal fluxes {vᵣ^{opt}: r ∈ [symbol] } |
| output: | Compass scores matrix {C}_{m′×n} (m′ ≤ m) |
| parameters: | Smoothing parameter λ ∈ [0, 1] (here, λ = 0.25) |
| | Nearest neighbor parameter k (here, k = 10) |
| | Optimality slack parameter ω ∈ [0, 1] (here, ω = 0.95) |
| | Penalty function p(x) (here, p(x) = 1/(1 + x)) |
| | Meta-reaction merging threshold ρ ∈ [0, 1] (see |

section 4.9)

| | |
|---|---|
| 1 | Compute with the procedures described in sections 4.6, 4.7: |

| | |
|---|---|
| 2 | a reaction expression matrix {R(G)}_{m×n} |
| 3 | a neighborhood reaction expression matrix {R^{N} (G)}_{m×n} |

| | |
|---|---|
| 4 | Transform reaction expressions to penalties: |

| | |
|---|---|
| 5 | P :=p(R(G)) |
| | P^{N} :=p(R^{N} (G)) |

| 6 | \hat{P} := (1 − λ)P + λP^{N} | |
|---|---|---|
| 7 | foreach r ∈ [symbol] , c ∈ [symbol] do | |
| 8 | \| | Let \hat{P}^{(c)} = (\hat{P}_{1,c}, . . . , \hat{P}_{m,c}) |
| 9 | \| | ${y}_{r , c} := {minimize }_{v ∈ {ℝ}^{m}} {\hat{P}}^{( c )} \cdot v$ |
| | \| | s.t. |
| | \| | (i) S · v = 0 |
| | \| | (ii) α ≤ v ≤ β |
| | \| | (iii) vᵣₑᵥ₍ᵣ₎ = 0 |
| | \| | (iv) vᵣ ≥ ω · vᵣ^{opt} |

| | |
|---|---|
| 10 | end |
| 11 | Let C^{raw} = {y_{r,c}}_{m×n} |
| 12 | Compute meta-reaction scores C^{meta-raw} = {y_{r,c}′}_{m″×n} (m″ ≤ m) as |
| | described in section 4.9 |
| 13 | Use Algorithm 3 to scale C^{meta-raw} and obtain C |
| 14 | Remove constant rows from C, defined as rows in which the |
| | difference between largest and smallest score is less than ε = 10⁻³ |
| 15 | return C |

Algorithm 3: Scale raw Compass scores

| | |
|---|---|
| input | : C^{meta-raw} |
| output | : C |

| | |
|---|---|
| 1 | C^{meta-raw} := − log(1 + C^{meta-raw}) |
| 2 | C := C^{meta-raw} − min (C^{meta-raw}) |
| 3 | return C |

### 5 Algorithm Generalization

One of the intuitions behind Compass is that the statistical power afforded by the number of observations (cells) in single-cell RNA-Seq allows increasing dimensionality by computing a new feature set based on the gene expression data and the GSMM. Here, Applicants used an intuitive set of objective functions for each reaction in the network, Applicants defined one objective function which is to maximize the flux it carries (recall that the network is unidirectional and therefore all reactions carry non-negative fluxes). This allows intuitive interpretation of the Compass scores as quantitative proxies to reaction activities. However, the algorithm can be generalized by using an arbitrary set of linear objective functions that pertain to cellular metabolism.

### 6 References

Baran et al., 2019. MetaCell: analysis of single-cell RNA-seq data using k-nn graph partitions. Genome Biol., 20(1):206.
Becker and Palsson, 2008. Context-Specific metabolic networks are consistent with experiments. PLoS Comput. Biol., 4(5):e1000082.
Chen et al., 2011. Shikonin and its analogs inhibit cancer cell glycolysis by targeting tumor pyruvate kinase-m2. Oncogene, 30(42):4297-4306.
Cole et al., 2019. Performance assessment and selection of normalization procedures for Single-Cell RNA-Seq. Cell Syst, 8(4):315-328.e8.
DeTomaso et al., 2019. Functional interpretation of single cell similarity maps. Nat. Commun., 10(1):4376.
Fletcher et al., 2017. Deconstructing olfactory stem cell trajectories at Single-Cell resolution. Cell Stem Cell, 20(6):817-830.e8.
Gaublomme et al., 2015. Single-Cell genomics unveils critical regulators of th17 cell pathogenicity. Cell, 163(6):1400-1412.
Jäger et al., 2009. Th1, th17, and th9 effector cells induce experimental autoimmune encephalomyelitis with different pathological phenotypes. J. Immunol., 183(11):7169-7177.
Kanehisa et al., 2017. KEGG: new perspectives on genomes, pathways, diseases and drugs. Nucleic Acids Res., 45(D1):D353-D361.
Law et al., 2014. voom: Precision weights unlock linear model analysis tools for RNA-seq read counts. Genome Biol., 15(2):R29.
Li et al., 2017. Identification of epigallocatechin-3-gallate as an inhibitor of phosphoglycerate mutase 1. Front. Pharmacol., 8:325.
Ritchie et al., 2015. limma powers differential expression analyses for RNA-sequencing and microarray studies. Nucleic Acids Res., 43(7):e47.
Schwartz and Pashko, 2004. Dehydroepiandrosterone, glucose-6-phosphate dehydrogenase, and longevity. Ageing Res. Rev., 3(2):171-187.
Shlomi et al., 2008. Network-based prediction of human tissue-specific metabolism. Nat. Biotechnol., 26(9):1003-1010.
Smyth, 2019. Limma Bioconductor Package. bioconductor.org/packages/release/bioc/html/limma.html. Bioconductor release 3.10; Last accessed Dec. 2019.
Thiele et al., 2013. A community-driven global reconstruction of human metabolism. Nat. Biotechnol., 31(5):419-425.
Tisdale and Threadgill, 1984. (+/−)2,3-dihydroxypropyl dichloroacetate, an inhibitor of glycerol kinase. Cancer Biochem. Biophys., 7(3):253-259.
Wang et al., 2015. CDSL/AIM regulates lipid biosynthesis and restrains th17 cell pathogenicity. Cell, 163(6):1413-1427.
Wang et al., 2017. Rational design of selective allosteric inhibitors of PHGDH and serine synthesis with anti-tumor activity. Cell Chem Biol, 24(1):55-65.
Westergaard et al., 1998. Characterization of glycerol uptake and glycerol kinase activity in rat hepatocytes cultured under different hormonal conditions. Biochim. Bio-phys. Acta, 1402(3):261-268.
Yosef et al., 2013. Dynamic regulatory network controlling TH17 cell differentiation. Nature, 496(7446):461-468.
Zhao et al., 2018. Shikonin inhibits tumor growth in mice by suppressing pyruvate kinase m2-mediated aerobic glycolysis. Sci. Rep., 8(1):14517.

### Example 9 Tables, see Wagner et al. 2020.

### Example 9 References

Adler, Miri, Yael Korem Kohanim, Avichai Tendler, Avi Mayo, and Uri Alon. 2019. “Continuum of Gene-Expression Profiles Provides Spatial Division of Labor within a Differentiated Cell Type.” Cell Systems 8 (1): 43-52.e5.
Aggarwal, Sudeepta, Nico Ghilardi, Ming-Hong Xie, Frederic J. de Sauvage, and Austin L. Gurney. 2003. “Interleukin-23 Promotes a Distinct CD4 T Cell Activation State Characterized by the Production of Interleukin-17.” The Journal of Biological Chemistry 278 (3): 1910-14.
Awasthi, Amit, Lorena Riol-Blanco, Anneli Jager, Thomas Korn, Caroline Pot, George Galileos, Estelle Bettelli, Vijay K. Kuchroo, and Mohamed Oukka. 2009. “Cutting Edge: IL-23 Receptor Gfp Reporter Mice Reveal Distinct Populations of IL-17-Producing Cells.” Journal of Immunology 182 (10): 5904-8.
Azizi, Elham, Ambrose J. Carr, George Plitas, Andrew E. Cornish, Catherine Konopacki, Sandhya Prabhakaran, Juozas Nainys, et al. 2018. “Single-Cell Map of Diverse Immune Phenotypes in the Breast Tumor Microenvironment.” Cell 174 (5): 1293-1308.e36.
Baran, Yael, Akhiad Bercovich, Arnau Sebe-Pedros, Yaniv Lubling, Amir Giladi, Elad Chomsky, Zohar Meir, Michael Hoichman, Aviezer Lifshitz, and Amos Tanay. 2019. “MetaCell: Analysis of Single-Cell RNA-Seq Data Using K-Nn Graph Partitions.” Genome Biology 20 (1): 206.
Beckermann, Kathryn E., Stephanie O. Dudzinski, and Jeffrey C. Rathmell. 2017. “Dysfunctional T Cell Metabolism in the Tumor Microenvironment.” Cytokine & Growth Factor Reviews 35 (June): 7-14.
Ben-Moshe, Noa Bossel, Shelly Hen-Avivi, Natalia Levitin, Dror Yehezkel, Marije Oosting, Leo A. B. Joosten, Mihai G. Netea, and Roi Avraham. 2019. “Predicting Bacterial Infection Outcomes Using Single Cell RNA-Sequencing Analysis of Human Immune Cells.” Nature Communications 10 (1): 3266.
Berod, Luciana, Christin Friedrich, Amrita Nandan, Jenny Freitag, Stefanie Hagemann, Kirsten Harmrolfs, Aline Sandouk, et al. 2014. “De Novo Fatty Acid Synthesis Controls the Fate between Regulatory T and T Helper 17 Cells.” Nature Medicine 20 (11): 1327-33.
Bordbar, Aarash, Jonathan M. Monk, Zachary A. King, and Bernhard O. Palsson. 2014. “Constraint-Based Models Predict Metabolic and Associated Cellular Functions.” Nature Reviews. Genetics 15 (2): 107-20.
Brown, Chrysothemis C., Herman Gudjonson, Yuri Pritykin, Deeksha Deep, Vincent-Philippe LavaHee, Alejandra Mendoza, Rachel Fromme, et al. 2019. “Transcriptional Basis of Mouse and Human Dendritic Cell Heterogeneity.” Cell 179 (4): 846-863.e24.
Buck, Michael D., Ryan T. Sowell, Susan M. Kaech, and Erika L. Pearce. 2017. “Metabolic Instruction of Immunity.” Cell 169 (4): 570-86.
Chapman, Nicole M., Mark R. Boothby, and Hongbo Chi. 2019. “Metabolic Coordination of T Cell Quiescence and Activation.” Nature Reviews. Immunology, August. doi.org/10.1038/s41577-019-0203-y.
Damiani, Chiara, Davide Maspero, Marzia Di Filippo, Riccardo Colombo, Dario Pescini, Alex Graudenzi, Hans Victor Westerhoff, Lilia Alberghina, Marco Vanoni, and Giancarlo Mauri. 2019. “Integration of Single-Cell RNA-Seq Data into Population Models to Characterize Cancer Metabolism.” PLoS Computational Biology 15 (2): e1006733.
Dijk, David van, Roshan Sharma, Juozas Nainys, Kristina Yim, Pooja Kathail, Ambrose J. Carr, Cassandra Burdziak, et al. 2018. “Recovering Gene Interactions from Single-Cell Data Using Data Diffusion.” Cell 174 (3): 716-729.e27.
Divakaruni, Ajit S., Wei Yuan Hsieh, Lucia Minarrieta, Tin N. Duong, Kristen K. O. Kim, Brandon R. Desousa, Alexander Y. Andreyev, et al. 2018. “Etomoxir Inhibits Macrophage Polarization by Disrupting CoA Homeostasis.” Cell Metabolism 28 (3): 490-503.e7.
Fabregat, Antonio, Steven Jupe, Lisa Matthews, Konstantinos Sidiropoulos, Marc Gillespie, Phani Garapati, Robin Haw, et al. 2018. “The Reactome Pathway Knowledgebase.” Nucleic Acids Research 46 (D1): D649-55.
Gaffen, Sarah L., Nydiaris Hernandez-Santos, and Alanna C. Peterson. 2011. “IL-17 Signaling in Host Defense against Candida Albicans.” Immunologic Research 50 (2-3): 181-87.
Gaublomme, Jellert T., Nir Yosef, Youjin Lee, Rona S. Gertner, Li V. Yang, Chuan Wu, Pier Paolo Pandolfi, et al. 2015. “Single-Cell Genomics Unveils Critical Regulators of Th17 Cell Pathogenicity.” Cell 163 (6): 1400-1412.
Geltink, Ramon I. Klein, Ryan L. Kyle, and Erika L. Pearce. 2018. “Unraveling the Complex Interplay Between T Cell Metabolism and Function.” Annual Review of Immunology 36 (April): 461-88.
Gemta, Lelisa F., Peter J. Siska, Marin E. Nelson, Xia Gao, Xiaojing Liu, Jason W. Locasale, Hideo Yagita, et al. 2019. “Impaired Enolase 1 Glycolytic Activity Restrains Effector Functions of Tumor-Infiltrating CD8+ T Cells.” Science Immunology 4 (31). doi. org/10.1126/sciimmunol.aap9520.
Gerriets, Valerie A., Rigel J. Kishton, Amanda G. Nichols, Andrew N. Macintyre, Makoto Inoue, Olga Ilkayeva, Peter S. Winter, et al. 2015. “Metabolic Programming and PDHK1 Control CD4+ T Cell Subsets and Inflammation.” The Journal of Clinical Investigation 125 (1): 194-207.
Ghoreschi, Kamran, Arian Laurence, Xiang-Ping Yang, Cristina M. Tato, Mandy J. McGeachy, Joanne E. Konkel, Haydeé L. Ramos, et al. 2010. “Generation of Pathogenic T(H)17 Cells in the Absence of TGF-β Signalling.” Nature 467 (7318): 967-71.
Green, Douglas R., Lorenzo Galluzzi, and Guido Kroemer. 2014. “Metabolic Control of Cell Death.” Science 345 (6203). doi.org/10.1126/science.1250256.
Grun, Dominic. 2019. “Revealing Dynamics of Gene Expression Variability in Cell State Space.” Nature Methods, November. doi.org/10.1038/s41592-019-0632-3.
Haghverdi, Laleh, Aaron T. L. Lun, Michael D. Morgan, and John C. Marioni. 2018. “Batch Effects in Single-Cell RNA-Sequencing Data Are Corrected by Matching Mutual Nearest Neighbors.” Nature Biotechnology 36 (5): 421-27.
Ho, Ping-Chih, and Susan M. Kaech. 2017. “Reenergizing T Cell Anti-Tumor Immunity by Harnessing Immunometabolic Checkpoints and Machineries.” Current Opinion in Immunology 46 (June): 38-44.
Hotamisligil, Gökhan S. 2017. “Foundations of Immunometabolism and Implications for Metabolic Health and Disease.” Immunity 47 (3): 406-20.
Huang, Mo, Jingshu Wang, Eduardo Torre, Hannah Dueck, Sydney Shaffer, Roberto Bonasio, John I. Murray, Arjun Raj, Mingyao Li, and Nancy R. Zhang. 2018. “SAVER: Gene Expression Recovery for Single-Cell RNA Sequencing.” Nature Methods 15 (7): 539-42.
Jha, Abhishek K., Stanley Ching-Cheng Huang, Alexey Sergushichev, Vicky Lampropoulou, Yulia Ivanova, Ekaterina Loginicheva, Karina Chmielewski, et al. 2015. “Network Integration of Parallel Metabolic and Transcriptional Data Reveals Metabolic Modules That Regulate Macrophage Polarization.” Immunity 42 (3): 419-30.
Kanehisa, Minoru, Miho Furumichi, Mao Tanabe, Yoko Sato, and Kanae Morishima. 2017. “KEGG: New Perspectives on Genomes, Pathways, Diseases and Drugs.” Nucleic Acids Research 45 (D1): D353-61.
Karmaus, Peer W. F., Xiang Chen, Seon Ah Lim, Andres A. Herrada, Thanh-Long M. Nguyen, Beisi Xu, Yogesh Dhungana, et al. 2019. “Metabolic Heterogeneity Underlies Reciprocal Fates of TH17 Cell Sternness and Plasticity.” Nature 565 (7737): 101-5.
Keren-Shaul, Hadas, Amit Spinrad, Assaf Weiner, Orit Matcovitch-Natan, Raz Dvir-Szternfeld, Tyler K. Ulland, Eyal David, et al. 2017. “A Unique Microglia Type Associated with Restricting Development of Alzheimer's Disease.” Cell 169 (7): 1276-1290.e17.
Kono, Michihito, Nobuya Yoshida, Kayaho Maeda, Nicole E. Skinner, Wenliang Pan, Vasileios C. Kyttaris, Maria G. Tsokos, and George C. Tsokos. 2018. “Pyruvate Dehydrogenase Phosphatase Catalytic Subunit 2 Limits Th17 Differentiation.” Proceedings of the National Academy of Sciences of the United States of America 115 (37): 9288-93.
Korn, Thomas, Estelle Bettelli, Mohamed Oukka, and Vijay K. Kuchroo. 2009. “IL-17 and Th17 Cells.” Annual Review of Immunology 27 (1): 485-517.
Lee, Youjin, Amit Awasthi, Nir Yosef, Francisco J. Quintana, Sheng Xiao, Anneli Peters, Chuan Wu, et al. 2012. “Induction and Molecular Signature of Pathogenic TH17 Cells.” Nature Immunology 13 (10): 991-99.
Lewis, Nathan E., Kim K. Hixson, Tom M. Conrad, Joshua A. Lerman, Pep Charusanti, Ashoka D. Polpitiya, Joshua N. Adkins, et al. 2010. “Omic Data from Evolved E. Coli Are Consistent with Computed Optimal Growth from Genome-Scale Models.” Molecular Systems Biology 6 (July). doi.org/10.1038/msb0.2010.47.
Lewis, Nathan E., Harish Nagaraj an, and Bernhard O. Palsson. 2012. “Constraining the Metabolic Genotype-Phenotype Relationship Using a Phylogeny of in Silico Methods.” Nature Reviews. Microbiology 10 (4): 291-305.
Lun, Aaron T. L., Karsten Bach, and John C. Marioni. 2016. “Pooling across Cells to Normalize Single-Cell RNA Sequencing Data with Many Zero Counts.” Genome Biology 17 (1): 1-14.
MacIver, Nancie J., Ryan D. Michalek, and Jeffrey C. Rathmell. 2013. “Metabolic Regulation of T Lymphocytes.” Annual Review of Immunology 31 (1): 259-83.
McGeachy, Mandy J., Yi Chen, Cristina M. Tato, Arian Laurence, Barbara Joyce-Shaikh, Wendy M. Blumenschein, Terrill K. McClanahan, John J. O'Shea, and Daniel J. Cua. 2009. “The Interleukin 23 Receptor Is Essential for the Terminal Differentiation of Interleukin 17-Producing Effector T Helper Cells in Vivo.” Nature Immunology 10 (3): 314-24.
Michalek, Ryan D., Valerie A. Gerriets, Sarah R. Jacobs, Andrew N. Macintyre, Nancie J. MacIver, Emily F. Mason, Sarah A. Sullivan, Amanda G. Nichols, and Jeffrey C. Rathmell. 2011. “Cutting Edge: Distinct Glycolytic and Lipid Oxidative Metabolic Programs Are Essential for Effector and Regulatory CD4+ T Cell Subsets.” Journal of Immunology 186 (6): 3299-3303.
Mills, Evanna L., Beth Kelly, Angela Logan, Ana S. H. Costa, Mukund Varma, Clare E. Bryant, Panagiotis Tourlomousis, et al. 2016. “Succinate Dehydrogenase Supports Metabolic Repurposing of Mitochondria to Drive Inflammatory Macrophages.” Cell 167 (2): 457-470.e13.
Mills, Evanna, and Luke A. J. O'Neill. 2014. “Succinate: A Metabolic Signal in Inflammation.” Trends in Cell Biology 24 (5): 313-20.
Miragaia, Ricardo J., Tomas Gomes, Agnieszka Chomka, Laura Jardine, Angela Riedel, Ahmed N. Hegazy, Natasha Whibley, et al. 2019. “Single-Cell Transcriptomics of Regulatory T Cells Reveals Trajectories of Tissue Adaptation.” Immunity 50 (2): 493-504.e7.
Newton, Ryan, Bhavana Priyadharshini, and Laurence A. Turka. 2016. “Immunometabolism of Regulatory T Cells.” Nature Immunology 17 (6): 618-25.
O'Brien, Edward J., Jonathan M. Monk, and Bernhard O. Palsson. 2015. “Using Genome-Scale Models to Predict Biological Capabilities.” Cell 161 (5): 971-87.
O'Neill, Luke A. J., Rigel J. Kishton, and Jeff Rathmell. 2016. “A Guide to Immunometabolism for Immunologists.” Nature Reviews. Immunology 16 (9): 553-65.
O'Neill, Luke A. J., and Edward J. Pearce. 2016. “Immunometabolism Governs Dendritic Cell and Macrophage FunctionMetabolic Regulation of Myeloid Cells.” The Journal of Experimental Medicine 213 (1): 15-23.
Opdam, Sjoerd, Anne Richelle, Benjamin Kellman, Shanzhong Li, Daniel C. Zielinski, and Nathan E. Lewis. 2017. “A Systematic Evaluation of Methods for Tailoring Genome-Scale Metabolic Models.” Cell Systems 4 (3): 318-329.e6.
Orth, Jeffrey D., Ines Thiele, and Bernhard O. Palsson. 2010. “What Is Flux Balance Analysis?” Nature Biotechnology 28 (3): 245-48.
Palsson, Bernhard ø. 2015. Systems Biology: Constraint-Based Reconstruction and Analysis. 2nd ed. edition. Cambridge University Press.
Paul, Franziska, Ya'ara Arkin, Amir Giladi, Diego Adhemar Jaitin, Ephraim Kenigsberg, Hadas Keren-Shaul, Deborah Winter, et al. 2015. “Transcriptional Heterogeneity and Lineage Commitment in Myeloid Progenitors.” Cell 163 (7): 1663-77.
Pearce, Edward J., and Bart Everts. 2015. “Dendritic Cell Metabolism.” Nature Reviews. Immunology 15 (1): 18-29.
Pearce, Erika L., Maya C. Poffenberger, Chih-Hao Chang, and Russell G. Jones. 2013. “Fueling Immunity: Insights into Metabolism and Lymphocyte Function.” Science 342 (6155). doi. org/10.1126/science. 1242454.
Puleston, Daniel J., Matteo Villa, and Erika L. Pearce. 2017. “Ancillary Activity: Beyond Core Metabolism in Immune Cells.” Cell Metabolism 26 (1): 131-41.
Raud, Brenda, Dominic G. Roy, Ajit S. Divakaruni, Tatyana N. Tarasenko, Raimo Franke, Eric H. Ma, Bozena Samborska, et al. 2018. “Etomoxir Actions on Regulatory and Memory T Cells Are Independent of Cpt1a-Mediated Fatty Acid Oxidation.” Cell Metabolism 28 (3): 504-515.e7.
Regev, Aviv, Sarah A. Teichmann, Eric S. Lander, Ido Amit, Christophe Benoist, Ewan Birney, Bernd Bodenmiller, et al. 2017. “Science Forum: The Human Cell Atlas.” ELife 6: e27041.
Rhoads, Jillian P., Amy S. Major, and Jeffrey C. Rathmell. 2017. “Fine Tuning of Immunometabolism for the Treatment of Rheumatic Diseases.” Nature Reviews. Rheumatology 13 (5): 313-20.
Romani, Luigina. 2011. “Immunity to Fungal Infections.” Nature Reviews. Immunology 11 (4): 275-88.
Russell, David G., Lu Huang, and Brian C. VanderVen. 2019. “Immunometabolism at the Interface between Macrophages and Pathogens.” Nature Reviews. Immunology, January. doi.org/10.1038/s41577-019-0124-9.
Sade-Feldman, Moshe, Keren Yizhak, Stacey L. Bjorgaard, John P. Ray, Carl G. de Boer, Russell W. Jenkins, David J. Lieb, et al. 2018. “Defining T Cell States Associated with Response to Checkpoint Immunotherapy in Melanoma.” Cell 175 (4): 998-1013.e20.
Satija, Rahul, Jeffrey A. Farrell, David Gennert, Alexander F. Schier, and Aviv Regev. 2015. “Spatial Reconstruction of Single-Cell Gene Expression Data.” Nature Biotechnology 33 (5): 495-502.
Shi, Lanbo, Qingkui Jiang, Yuri Bushkin, Selvakumar Subbian, and Sanjay Tyagi. 2019. “Biphasic Dynamics of Macrophage Immunometabolism during Mycobacterium Tuberculosis Infection.” MBio 10 (2). doi.org/10.1128/mBio.02550-18.
Shi, Lewis Z., Ruoning Wang, Gonghua Huang, Peter Vogel, Geoffrey Neale, Douglas R. Green, and Hongbo Chi. 2011. “HIF1alpha-Dependent Glycolytic Pathway Orchestrates a Metabolic Checkpoint for the Differentiation of TH17 and Treg Cells.” The Journal of Experimental Medicine 208 (7): 1367-76.
Soldatov, Ruslan, Marketa Kaucka, Maria Eleni Kastriti, Julian Petersen, Tatiana Chontorotzea, Lukas Englmaier, Natalia Akkuratova, et al. 2019. “Spatiotemporal Structure of Cell Fate Decisions in Murine Neural Crest.” Science 364 (6444). doi.org/10.1126/science.aas9536.
Stockinger, Brigitta, and Sara Omenetti. 2017. “The Dichotomous Nature of T Helper 17 Cells.” Nature Reviews. Immunology 17 (9): 535-44.
Subramanian, Aravind, Pablo Tamayo, Vamsi K. Mootha, Sayan Mukherjee, Benjamin L. Ebert, Michael A. Gillette, Amanda Paulovich, et al. 2005. “Gene Set Enrichment Analysis: A Knowledge-Based Approach for Interpreting Genome-Wide Expression Profiles.” Proceedings of the National Academy of Sciences of the United States of America 102 (43): 15545-50.
Sundrud, Mark S., Sergei B. Koralov, Markus Feuerer, Dinis Pedro Calado, Aimee Elhed Kozhaya, Ava Rhule-Smith, Rachel E. Lefebvre, et al. 2009. “Halofuginone Inhibits T(H)17 Cell Differentiation by Activating the Amino Acid Starvation Response.” Science 324 (5932): 1334-38.
Svensson, Valentine, Roser Vento-Tormo, and Sarah A. Teichmann. 2018. “Exponential Scaling of Single-Cell RNA-Seq in the Past Decade.” Nature Protocols 13 (4): 599-604.
Tanay, Amos, and Aviv Regev. 2017. “Scaling Single-Cell Genomics from Phenomenology to Mechanism.” Nature 541 (7637): 331-38.
Tesmer, Laura A., Steven K. Lundy, Sujata Sarkar, and David A. Fox. 2008. “Th17 Cells in Human Disease.” Immunological Reviews 223 (June): 87-113.
Thiele, Ines, Neil Swainston, Ronan M. T. Fleming, Andreas Hoppe, Swagatika Sahoo, Maike K. Aurich, Hulda Haraldsdottir, et al. 2013. “A Community-Driven Global Reconstruction of Human Metabolism.” Nature Biotechnology 31 (5): 419-25.
Vallejos, Catalina A., John C. Marioni, and Sylvia Richardson. 2015. “BASiCS: Bayesian Analysis of Single-Cell Sequencing Data.” PLoS Computational Biology 11 (6): e1004333.
Van den Bossche, Jan, Luke A. O'Neill, and Deepthi Menon. 2017. “Macrophage Immunometabolism: Where Are Applicants (Going)?” Trends in Immunology 38 (6): 395-406.
Van Hove, Hannah, Liesbet Martens, Isabelle Scheyltj ens, Karen De Vlaminck, Ana Rita Pombo Antunes, Sofie De Prijck, Niels Vandamme, et al. 2019. “A Single-Cell Atlas of Mouse Brain Macrophages Reveals Unique Transcriptional Identities Shaped by Ontogeny and Tissue Environment.” Nature Neuroscience 22 (6): 1021-35.
Varma, Amit, and Bernhard O. Palsson. 1994. “Metabolic Flux Balancing: Basic Concepts, Scientific and Practical Use.” Nature Biotechnology 12 (10): 994-98.
Vento-Tormo, Roser, Mirjana Efremova, Rachel A. Botting, Margherita Y. Turco, Miguel Vento-Tormo, Kerstin B. Meyer, Jong-Eun Park, et al. 2018. “Single-Cell Reconstruction of the Early Maternal-Fetal Interface in Humans.” Nature 563 (7731): 347-53.
Vieira Braga, Felipe A., Gozde Kar, Marijn Berg, Orestes A. Carpaij, Krzysztof Polanski, Lukas M. Simon, Sharon Brouwer, et al. 2019. “A Cellular Census of Human Lungs Identifies Novel Cell States in Health and in Asthma.” Nature Medicine 25 (7): 1153-63.
Wagner, Allon, Aviv Regev, and Nir Yosef. 2016. “Revealing the Vectors of Cellular Identity with Single-Cell Genomics.” Nature Biotechnology 34 (11): 1145-60.
Wagner, Florian, Yun Yan, and Itai Yanai. 2018. “K-Nearest Neighbor Smoothing for High-Throughput Single-Cell RNA-Seq Data.” BioRxiv. doi.org/10. 1101/217737.
Wang, Chao, Nir Yosef, Jellert Gaublomme, Chuan Wu, Youjin Lee, Clary B. Clish, Jim Kaminski, et al. 2015. “CD5L/AIM Regulates Lipid Biosynthesis and Restrains Th17 Cell Pathogenicity.” Cell 163 (6): 1413-27.
Wu, P., P. V. Blair, J. Sato, J. Jaskiewicz, K. M. Popov, and R. A. Harris. 2000. “Starvation Increases the Amount of Pyruvate Dehydrogenase Kinase in Several Mammalian Tissues.” Archives of Biochemistry and Biophysics 381 (1): 1-7.
Wu, P., J. M. Peters, and R. A. Harris. 2001. “Adaptive Increase in Pyruvate Dehydrogenase Kinase 4 during Starvation Is Mediated by Peroxisome Proliferator-Activated Receptor Alpha.” Biochemical and Biophysical Research Communications 287 (2): 391-96.
Wu, Xinyu, Jie Tian, and Shengjun Wang. 2018. “Insight Into Non-Pathogenic Th17 Cells in Autoimmune Diseases.” Frontiers in Immunology 9 (May): 1112.
Yosef, Nir, Alex K. Shalek, Jellert T. Gaublomme, Hulin Jin, Youjin Lee, Amit Awasthi, Chuan Wu, et al. 2013. “Dynamic Regulatory Network Controlling TH17 Cell Differentiation.” Nature 496 (7446): 461-68.
Zanini, Fabio, Makeda L. Robinson, Derek Croote, Malaya Kumar Sahoo, Ana Maria Sanz, Eliana Ortiz-Lasso, Ludwig Luis Albornoz, et al. 2018. “Virus-Inclusive Single-Cell RNA Sequencing Reveals the Molecular Signature of Progression to Severe Dengue.” Proceedings of the National Academy of Sciences of the United States of America 115 (52): E12363-69.
Zhou, Liang, Ivaylo I. Ivanov, Rosanne Spolski, Roy Min, Kevin Shenderov, Takeshi Egawa, David E. Levy, Warren J. Leonard, and Dan R. Littman. 2007. “IL-6 Programs T(H)-17 Cell Differentiation by Promoting Sequential Engagement of the IL-21 and IL-23 Pathways.” Nature Immunology 8 (9): 967-74.

### Tables

TABLE 1
Compass prediction of all reactions in the glycolysis pathway correlated
to Th17 pathogenicity. The reactions are listed as most positively correlated
to most negatively correlated (positive score to negative score).

| rxn_name_long | genes_associated_with_rxn | Score |
|---|---|---|
| taurocholate transport via bicarbonate | SLCO1A1; | 0.23 |
| countertransport | SLCO1B2; | |
| | SLCO4A1 | |
| T4 transport via bicarbonate countertransport | SLCO1A1; | 0.23 |
| | SLCO1B2; | |
| | SLCO1C1; | |
| | SLCO4A1 | |
| T4 transport via facilitated diffusion | SLC16A2 | 0.23 |
| T3 transport via bicarbonate countertransport | SLCO1A1; | 0.23 |
| | SLCO1B2; | |
| | SLCO1C1; | |
| | SLCO4A1 | |
| T3 transport via facilitated diffusion | SLC16A2 | 0.23 |
| AMP/ATP transporter, endoplasmic reticulum | | 0.23 |
| fatty-acid--CoA ligase | ACSBG2; ACSL1; | 0.23 |
| | ACSL3; ACSL4; | |
| | ACSL5; ACSL6 | |
| EC: 6.2.1.3 | ACSBG2; ACSL1; | 0.23 |
| | ACSL3; ACSL4; | |
| | ACSL5; ACSL6; | |
| | SLC27A2 | |
| EC: 6.2.1.3 | ACSBG2; ACSL1; | 0.23 |
| | ACSL3; ACSL4; | |
| | ACSL5; ACSL6; | |
| | SLC27A2 | |
| Utilized transport | | 0.23 |
| alpha-Linolenic acid exchange | | 0.23 |
| taurocholate transport via sodium cotransport | SLC10A1; | 0.22 |
| | SLC10A2 | |
| estrone-3-sulfate transport via sodium | SLC10A1 | 0.22 |
| cotransport | | |
| Resistance-Nodulation-Cell Division (RND) | SLCO1A1 | 0.22 |
| TCDB: 2.A.60.1.14 | | |
| taurocholate transport via bicarbonate | SLCO1A1; | 0.22 |
| countertransport | SLCO1B2; | |
| | SLCO4A1 | |
| Resistance-Nodulation-Cell Division (RND) | SLCO1A1 | 0.22 |
| TCDB: 2.A.60.1.14 | | |
| Chenodeoxyglycocholate exchange | | 0.22 |
| Resistance-Nodulation-Cell Division (RND) | SLCO1A1 | 0.22 |
| TCDB: 2.A.60.1.14 | | |
| Resistance-Nodulation-Cell Division (RND) | SLCO1A1 | 0.22 |
| TCDB: 2.A.60.1.14 | | |
| Na(+)/bile acid cotransporter Active transport | SLC10A1 | 0.21 |
| Resistance-Nodulation-Cell Division (RND) | SLCO1A1 | 0.21 |
| TCDB: 2.A.60.1.14 | | |
| Resistance-Nodulation-Cell Division (RND) | SLCO1A1 | 0.21 |
| TCDB: 2.A.60.1.14 | | |
| Resistance-Nodulation-Cell Division (RND) | SLCO1A1 | 0.21 |
| TCDB: 2.A.60.1.14 | | |
| bile acid intracellular transport | | 0.21 |
| bile acid intracellular transport | | 0.20 |
| T4 transport via bicarbonate countertransport | SLCO1A1; | 0.20 |
| | SLCO1B2; | |
| | SLCO1C1; | |
| | SLCO4A1 | |
| T4 transport via facilitated diffusion | SLC16A2 | 0.20 |
| T3 transport via bicarbonate countertransport | SLCO1A1; | 0.20 |
| | SLCO1B2; | |
| | SLCO1C1; | |
| | SLCO4A1 | |
| T3 transport via facilitated diffusion | SLC16A2 | 0.20 |
| estrone 3-sulfate transport via bicarbonate | SLCO1A1; | 0.20 |
| countertransport | SLCO1B2; | |
| | SLCO1C1; | |
| | SLCO2B1; | |
| | SLCO3A1; | |
| | SLCO4A1 | |
| Organic anion transporter 5 Utilized transport | | 0.20 |
| prostaglandin transport via bicarbonate | SLCO1A1; | 0.20 |
| countertransport | SLCO2A1; | |
| | SLCO3A1; | |
| | SLCO4A1 | |
| prostaglandin uniport | SLC22A1; | 0.20 |
| | SLC22A2 | |
| UDPglucose: alpha-D-galactose-1-phosphate | CBS | 0.19 |
| uridylyltransferase Galactose metabolism/ | | |
| Nucleotide sugars metabolism EC: 2.7.7.12 | | |
| ATP: D-galactose 1-phosphotransferase Galactose | | 0.19 |
| metabolism EC: 2.7.1.6 | | |
| Hydroxymethylglutaryl CoA reductase (ir) in | HMGCR | 0.19 |
| cytosol | | |
| (R)-Mevalonate: NADP+ oxidoreductase (CoA | HMGCR | 0.19 |
| acylating) Biosynthesis of steroids EC: 1.1.1.34 | | |
| R total 3 position exchange | | 0.19 |
| triacylglycerol (homo sapiens) exchange | | 0.19 |
| estrone 3-sulfate transport via bicarbonate | SLCO1A1; | 0.18 |
| countertransport | SLCO1B2; | |
| | SLCO1C1; | |
| | SLCO2B1; | |
| | SLCO3A1; | |
| | SLCO4A1 | |
| prostaglandin-a2 exchange | | 0.18 |
| prostaglandin-b2 exchange | | 0.18 |
| prostaglandin-c2 exchange | | 0.18 |
| RE2069 | | 0.18 |
| RE3566 | | 0.18 |
| RE3567 | | 0.18 |
| cholesterol intracellular transport | STARD3 | 0.18 |
| cholesterol intracellular transport | STARD3 | 0.18 |
| cholesterol intracellular transport | | 0.18 |
| cholesterol intracellular transport | | 0.18 |
| cholesterol intracellular transport | | 0.18 |
| Vesicular transport | | 0.18 |
| Vesicular transport | | 0.18 |
| Vesicular transport | | 0.18 |
| phosphate transport in/out via three Na+ | SLC17A1; | 0.17 |
| symporter | SLC17A2; | |
| | SLC17A3; | |
| | SLC17A4; | |
| | SLC34A1; | |
| | SLC34A2 | |
| Facilitated diffusion | | 0.17 |
| glycocholate transport via sodium cotransport | SLC10A1; | 0.17 |
| | SLC10A2 | |
| Exchange of hydrosulfide | | 0.17 |
| Free diffusion | | 0.17 |
| 3HCO3_NAt | SLC4A4; SLC4A5 | 0.17 |
| EC: 6.2.1.3 | ACSBG2; ACSL1; | 0.17 |
| | ACSL3; ACSL4; | |
| | ACSL5; ACSL6; | |
| | SLC27A2 | |
| Transport reaction | | 0.17 |
| Utilized transport | | 0.17 |
| bilirubin transport via bicarbonate | SLCO1B2 | 0.17 |
| countertransport | | |
| Prostaglandin E2 exchange | | 0.16 |
| Glycoside-Pentoside-Hexuronide (GPH): Cation | SLC10A1 | 0.16 |
| Symporter TCDB: 2.A.28.1.1 | | |
| Glycoside-Pentoside-Hexuronide (GPH): Cation | SLC10A1 | 0.16 |
| Symporter TCDB: 2.A.28.1.1 | | |
| Resistance-Nodulation-Cell Division (RND) | SLCO1A1 | 0.16 |
| TCDB: 2.A.60.1.14 | | |
| Resistance-Nodulation-Cell Division (RND) | SLCO1A1 | 0.16 |
| TCDB: 2.A.60.1.14 | | |
| cholate transport via bicarbonate | SLCO1A1; | 0.16 |
| countertransport | SLCO1B2 | |
| cholate transport via sodium cotransport | SLC10A1; | 0.16 |
| | SLC10A2 | |
| Anion Exchanger (AE) TCDB: 2.A.31.2.8 | SLC4A5 | 0.16 |
| Cys-Gly transport in via proton symport | SLC15A2 | 0.16 |
| gamma-glutamyltranspeptidase Glutathione | GGT1; GGT5; | 0.16 |
| metabolism EC: 3.4.11.4 | GGT6; GGT7 | |
| gamma-glutamyltranspeptidase Glutathione | GGT1; GGT5; | 0.16 |
| metabolism EC: 3.4.11.4 | GGT6; GGT7 | |
| ribulose 5-phosphate 3-epimerase | RPE | 0.16 |
| ribulose 5-phosphate 3-epimerase | RPE | 0.16 |
| estradiol glucuronide transport via bicarbonate | SLCO1A1; | 0.16 |
| countertransport | SLCO1B2; | |
| | SLCO1C1; | |
| | SLCO4A1 | |
| 17-beta-D-glucuronide transport (ATP- | ABCC4 | 0.16 |
| dependent) | | |
| phosphate transport in/out via two Na+ | SLC34A3 | 0.15 |
| symporter | | |
| Citrate exchange | | 0.15 |
| p-cumic alcohol: NAD+ oxidoreductase Glycine, | ALDH7A1 | 0.15 |
| serine and threonine metabolism EC: 1.2.1.8 | | |
| p-Cumic alcohol: NADP+ oxidoreductase Glycine, | ALDH7A1 | 0.15 |
| serine and threonine metabolism EC: 1.2.1.8 | | |
| p-cumic alcohol: NAD+ oxidoreductase Glycine, | ALDH7A1 | 0.15 |
| serine and threonine metabolism EC: 1.2.1.8 | | |
| p-Cumic alcohol: NADP+ oxidoreductase Glycine, | ALDH7A1 | 0.15 |
| serine and threonine metabolism EC: 1.2.1.8 | | |
| bicarbonate transport (Na/HCO3 cotransport) | SLC4A7 | 0.15 |
| glucose-6-phosphate dehydrogenase, | H6PD | 0.15 |
| endoplasmic reticulum | | |
| glucose 6-phosphate dehydrogenase, | G6PD2; H6PD | 0.15 |
| endoplasmic reticulum | | |
| 11-beta-hydroxysteroid dehydrogenase type 1 | HSD11B1 | 0.15 |
| 11-beta-hydroxysteroid dehydrogenase type 2 | HSD11B2 | 0.15 |
| glucose-6-phosphate dehydrogenase, | H6PD | 0.15 |
| endoplasmic reticulum | | |
| glucose 6-phosphate dehydrogenase, | G6PD2; H6PD | 0.15 |
| endoplasmic reticulum | | |
| 11-beta-hydroxysteroid dehydrogenase type 1 | HSD11B1 | 0.15 |
| 11-beta-hydroxysteroid dehydrogenase type 2 | HSD11B2 | 0.15 |
| Resistance-Nodulation-Cell Division (RND) | SLCO2A1 | 0.15 |
| TCDB: 2.A.60.1.2 | | |
| Resistance-Nodulation-Cell Division (RND) | SLCO1A1 | 0.15 |
| TCDB: 2.A.60.1.14 | | |
| Organic anion transporter 5 Utilized transport | | 0.15 |
| prostaglandin transport via bicarbonate | SLCO1A1; | 0.15 |
| countertransport | SLCO2A1; | |
| | SLCO3A1; | |
| | SLCO4A1 | |
| prostaglandin uniport | SLC22A1; | 0.15 |
| | SLC22A2 | |
| bicarbonate transport (Cl—/HCO3— exchange) | SLC4A1; | 0.15 |
| | SLC4A2; | |
| | SLC4A3; SLC4A9 | |
| glycocholate transport via bicarbonate | SLCO1A1; | 0.15 |
| countertransport | SLCO1B2 | |
| Resistance-Nodulation-Cell Division (RND) | SLCO1A1 | 0.15 |
| TCDB: 2.A.60.1.14 | | |
| ATP-binding Cassette (ABC) TCDB: 3.A.1.208.8 | ABCC1 | 0.15 |
| Dopamine reversible transport in via sodium | SLC6A2; SLC6A3 | 0.14 |
| symport (1:2) | | |
| Dopamine uniport | SLC22A2; | 0.14 |
| | SLC22A3; | |
| | SLC22A5 | |
| chloride transport via oxalate countertransport | SLC26A6 | 0.14 |
| (2:1) | | |
| oxalate transport via bicarbonate | SLC26A1; | 0.14 |
| countertransport | SLC26A2; | |
| | SLC26A3 | |
| L-ascorbate transport via facilitated diffusion | | 0.14 |
| L-ascorbate transport via proton symport | SLC23A1; | 0.14 |
| | SLC23A2 | |
| Nucleobase: Cation Symporter-2 (NCS2) | SLC23A1 | 0.14 |
| TCDB: 2.A.40.6.1 | | |
| choline phosphate phosphatase | PHOSPHO1 | 0.14 |
| Choline kinase | CHKA; CHKB | 0.14 |
| ABC bile acid transporter | ABCB11; ABCC3 | 0.14 |
| Oxalosuccinate: NADP+ oxidoreductase | IDH1 | 0.14 |
| (decarboxylating) Citrate cycle (TCA cycle) | | |
| EC: 1.1.1.42 | | |
| Isocitrate: NADP+ oxidoreductase | IDH1; IDH2 | 0.14 |
| (decarboxylating) Citrate cycle (TCA cycle) | | |
| EC: 1.1.1.42 | | |
| isocitrate dehydrogenase (NADP) | IDH1 | 0.14 |
| retinol dehydrogenase (9-cis, NADH) | RDH5 | 0.14 |
| bilirubin transport via bicarbonate | SLCO1B2 | 0.14 |
| countertransport | | |
| L-Cysteine L-homocysteine-lyase (deaminating) | CTH | 0.14 |
| Cysteine metabolism EC: 4.4.1.1 | | |
| Resistance-Nodulation-Cell Division (RND) | SLCO1A1 | 0.13 |
| TCDB: 2.A.60.1.14 | | |
| Histamine uniport | SLC22A2; | 0.13 |
| | SLC22A3; | |
| | SLC22A5 | |
| dehydroepiandrosterone sulfate transport via | SLCO1A1; | 0.13 |
| bicarbonate countertransport | SLCO1B2; | |
| | SLCO2B1 | |
| Organic anion transporter 5 Utilized transport | | 0.13 |
| bilirubin beta-diglucuronide transport via | SLCO1B2 | 0.13 |
| bicarbonate countertransport | | |
| bilirubin monoglucuronide transport via | SLCO1B2 | 0.13 |
| bicarbonate countertransport | | |
| glycocholate transport via bicarbonate | SLCO1A1; | 0.13 |
| countertransport | SLCO1B2 | |
| bile acid intracellular transport | | 0.13 |
| leukotriene C4 transport via bicarbonate | SLCO1B2 | 0.13 |
| countertransport | | |
| prostaglandin transport via bicarbonate | SLCO2A1 | 0.13 |
| countertransport | | |
| prostaglandin transport via bicarbonate | SLCO2A1 | 0.13 |
| countertransport | | |
| Prostaglandin H2 transport | | 0.13 |
| Prostaglandin I2 transport | | 0.13 |
| Organic anion transporter 5 Utilized transport | | 0.13 |
| Organic anion transporter 5 Utilized transport | | 0.13 |
| Organic anion transporter 5 Utilized transport | | 0.13 |
| Organic anion transporter 5 Utilized transport | | 0.13 |
| Organic anion transporter 5 Utilized transport | | 0.13 |
| Organic anion transporter 5 Utilized transport | | 0.13 |
| Organic anion transporter 5 Utilized transport | | 0.13 |
| Leukotriene D4 dipeptidase | | 0.13 |
| Resistance-Nodulation-Cell Division (RND) | SLCO2A1 | 0.13 |
| TCDB: 2.A.60.1.2 | | |
| Resistance-Nodulation-Cell Division (RND) | SLCO2A1 | 0.13 |
| TCDB: 2.A.60.1.2 | | |
| Resistance-Nodulation-Cell Division (RND) | SLCO1A1 | 0.13 |
| TCDB: 2.A.60.1.14 | | |
| Resistance-Nodulation-Cell Division (RND) | SLCO2A1 | 0.13 |
| TCDB: 2.A.60.1.2 | | |
| Resistance-Nodulation-Cell Division (RND) | SLCO2A1 | 0.13 |
| TCDB: 2.A.60.1.2 | | |
| Resistance-Nodulation-Cell Division (RND) | SLCO2A1 | 0.13 |
| TCDB: 2.A.60.1.2 | | |
| Resistance-Nodulation-Cell Division (RND) | SLCO1B2 | 0.13 |
| TCDB: 2.A.60.1.5 | | |
| Resistance-Nodulation-Cell Division (RND) | SLCO1A1 | 0.13 |
| TCDB: 2.A.60.1.14 | | |
| Resistance-Nodulation-Cell Division (RND) | SLCO2A1 | 0.13 |
| TCDB: 2.A.60.1.2 | | |
| Resistance-Nodulation-Cell Division (RND) | SLCO2A1 | 0.13 |
| TCDB: 2.A.60.1.2 | | |
| Resistance-Nodulation-Cell Division (RND) | SLCO2A1 | 0.13 |
| TCDB: 2.A.60.1.2 | | |
| leukotriene A4 transport | | 0.13 |
| leukotriene B4 transport | | 0.13 |
| leukotriene D4 transport | | 0.13 |
| leukotriene E4 transport | | 0.13 |
| leukotriene F4 transport | | 0.13 |
| prostaglandin uniport | SLC22A1; | 0.13 |
| | SLC22A2 | |
| Resistance-Nodulation-Cell Division (RND) | SLCO1A1 | 0.13 |
| TCDB: 2.A.60.1.14 | | |
| Resistance-Nodulation-Cell Division (RND) | SLCO2A1 | 0.13 |
| TCDB: 2.A.60.1.2 | | |
| Major Facilitator(MFS) TCDB: 2.A.1.19.1 | SLC22A1 | 0.13 |
| Organic anion transporter 5 Utilized transport | | 0.13 |
| Organic anion transporter 5 Utilized transport | | 0.13 |
| Organic anion transporter 5 Utilized transport | | 0.13 |
| Organic anion transporter 5 Utilized transport | | 0.13 |
| Organic anion transporter 5 Utilized transport | | 0.13 |
| Organic anion transporter 5 Utilized transport | | 0.13 |
| Organic anion transporter 5 Utilized transport | | 0.13 |
| Organic anion transporter 5 Utilized transport | | 0.13 |
| Organic anion transporter 5 Utilized transport | | 0.13 |
| Organic anion transporter 5 Utilized transport | | 0.13 |
| Organic anion transporter 5 Utilized transport | | 0.13 |
| Resistance-Nodulation-Cell Division (RND) | SLCO2A1 | 0.13 |
| TCDB: 2.A.60.1.2 | | |
| Resistance-Nodulation-Cell Division (RND) | SLCO2A1 | 0.13 |
| TCDB: 2.A.60.1.2 | | |
| Resistance-Nodulation-Cell Division (RND) | SLCO2A1 | 0.13 |
| TCDB: 2.A.60.1.2 | | |
| Resistance-Nodulation-Cell Division (RND) | SLCO2A1 | 0.13 |
| TCDB: 2.A.60.1.2 | | |
| Resistance-Nodulation-Cell Division (RND) | SLCO2A1 | 0.13 |
| TCDB: 2.A.60.1.2 | | |
| Organic anion transporter 5 Utilized transport | | 0.13 |
| Resistance-Nodulation-Cell Division (RND) | SLCO1B2 | 0.13 |
| TCDB: 2.A.60.1.5 | | |
| Resistance-Nodulation-Cell Division (RND) | SLCO2A1 | 0.13 |
| TCDB: 2.A.60.1.2 | | |
| Resistance-Nodulation-Cell Division (RND) | SLCO2A1 | 0.13 |
| TCDB: 2.A.60.1.2 | | |
| Organic anion transporter 5 Utilized transport | | 0.13 |
| Resistance-Nodulation-Cell Division (RND) | SLCO2A1 | 0.13 |
| TCDB: 2.A.60.1.2 | | |
| Organic anion transporter 5 Utilized transport | | 0.13 |
| Resistance-Nodulation-Cell Division (RND) | SLCO2A1 | 0.13 |
| TCDB: 2.A.60.1.2 | | |
| Organic anion transporter 5 Utilized transport | | 0.13 |
| bicarbonate transport (HCl/NaHCO3 exchange) | SLC4A10; | 0.13 |
| | SLC4A8 | |
| Utilized transport | | 0.13 |
| Resistance-Nodulation-Cell Division (RND) | SLCO1A1 | 0.13 |
| TCDB : 2.A.60.1.14 | | |
| Resistance-Nodulation-Cell Division (RND) | SLCO1A1 | 0.13 |
| TCDB: 2.A.60.1.14 | | |
| Resistance-Nodulation-Cell Division (RND) | SLCO1A1 | 0.13 |
| TCDB: 2.A.60.1.14 | | |
| Resistance-Nodulation-Cell Division (RND) | SLCO1A1 | 0.13 |
| TCDB: 2.A.60.1.14 | | |
| Resistance-Nodulation-Cell Division (RND) | SLCO1B2 | 0.13 |
| TCDB: 2.A.60.1.5 | | |
| Resistance-Nodulation-Cell Division (RND) | SLCO1B2 | 0.13 |
| TCDB: 2.A.60.1.5 | | |
| Resistance-Nodulation-Cell Division (RND) | SLCO1B2 | 0.13 |
| TCDB: 2.A.60.1.5 | | |
| Resistance-Nodulation-Cell Division (RND) | SLCO2A1 | 0.13 |
| TCDB: 2.A.60.1.2 | | |
| Resistance-Nodulation-Cell Division (RND) | SLCO2A1 | 0.13 |
| TCDB: 2.A.60.1.2 | | |
| Resistance-Nodulation-Cell Division (RND) | SLCO2A1 | 0.13 |
| TCDB: 2.A.60.1.2 | | |
| Resistance-Nodulation-Cell Division (RND) | SLCO2A1 | 0.13 |
| TCDB: 2.A.60.1.2 | | |
| 2-methylpropanoyl-CoA: enzyme N6- | DBT | 0.13 |
| (dihydrolipoyl)lysine S-(2- | | |
| methylpropanoyl)transferase Valine, leucine and | | |
| isoleucine degradation EC: 2.3.1.168 | | |
| RE3326 | BCKDHB | 0.13 |
| Serotonin uniport | SLC22A1; | 0.13 |
| | SLC22A2 | |
| retinol dehydrogenase (all-trans) | RDH16F1; RDH5 | 0.13 |
| RE2651 | | 0.13 |
| Resistance-Nodulation-Cell Division (RND) | SLCO1A1 | 0.13 |
| TCDB: 2.A.60.1.14 | | |
| Resistance-Nodulation-Cell Division (RND) | SLCO1A1 | 0.13 |
| TCDB: 2.A.60.1.14 | | |
| Resistance-Nodulation-Cell Division (RND) | SLCO2A1 | 0.13 |
| TCDB: 2.A.60.1.2 | | |
| adenosine transport (Na/Adn cotransport) | SLC28A1; | 0.13 |
| | SLC28A2; | |
| | SLC28A3 | |
| guanosine facilated transport in cytosol | SLC29A1; | 0.13 |
| | SLC29A2 | |
| guanosine transport in via sodium symport | SLC28A2; | 0.13 |
| | SLC28A3 | |
| inosine transport in via sodium symport | SLC28A2; | 0.13 |
| | SLC28A3 | |
| uridine facilated transport in cytosol | SLC29A1; | 0.13 |
| | SLC29A2 | |
| uridine transport in via sodium symport | SLC28A1; | 0.13 |
| | SLC28A2; | |
| | SLC28A3 | |
| dADP transport via dCDP antiport | SLC25A19 | 0.13 |
| dADP transport via dGDP antiport | SLC25A19 | 0.13 |
| dUDP transport via dTDP antiport | SLC25A19 | 0.13 |
| dUDP transport via dGDP antiport | SLC25A19 | 0.13 |
| dUDP transport via dADP antiport | SLC25A19 | 0.13 |
| dUDP transport via dCDP antiport | SLC25A19 | 0.13 |
| dTDP transport via dUDP antiport | SLC25A19 | 0.13 |
| dTDP transport via dGTP antiport | SLC25A19 | 0.13 |
| dTDP transport via dADP antiport | SLC25A19 | 0.13 |
| dTDP transport via dCDP antiport | SLC25A19 | 0.13 |
| dCDP transport via dUDP antiport | SLC25A19 | 0.13 |
| dCDP transport via dTDP antiport | SLC25A19 | 0.13 |
| dCDP transport via dGDP antiport | SLC25A19 | 0.13 |
| dCDP transport via dADP antiport | SLC25A19 | 0.13 |
| dGDP transport via dUDP antiport | SLC25A19 | 0.13 |
| dGDP transport via dTDP antiport | SLC25A19 | 0.13 |
| dGDP transport via dADP antiport | SLC25A19 | 0.13 |
| dGDP transport via dCDP antiport | SLC25A19 | 0.13 |
| dADP transport via dUDP antiport | SLC25A19 | 0.13 |
| dADP transport via dTDP antiport | SLC25A19 | 0.13 |
| diffusion of aspartate into blood | | 0.12 |
| OROTGLUt | SLC22A7 | 0.12 |
| Postulated transport reaction | | 0.12 |
| choline, sodium cotransport | SLC5A7 | 0.12 |
| Choline uniport | SLC22A2; | 0.12 |
| | SLC22A5 | |
| L-carnitine reversible transport | SLC22A5 | 0.12 |
| L-carnitine inward transport by Na+ symport | SLC22A5 | 0.12 |
| chloride transport via bicarbonate | SLC26A6 | 0.12 |
| countertransport (2:1) | | |
| Diacylglycerol phosphate kinase (homo sapiens) | DGKI; DGKZ | 0.12 |
| phosphatidate transport, nuclear | | 0.12 |
| Active transport | | 0.12 |
| nucleoside-diphosphate kinase (ATP: CDP) | GM20390; | 0.12 |
| | NME2; NME3; | |
| | NME6; NME7 | |
| nucleoside-diphosphate kinase (ATP: GDP) | GM20390; | 0.12 |
| | NME2; NME3; | |
| | NME6; NME7 | |
| cytidylate kinase (CMP) | CMPK1 | 0.12 |
| cytidylate kinase (CMP) | CMPK1 | 0.12 |
| cytidylate kinase (CMP, dGTP) | CMPK1 | 0.12 |
| cytidylate kinase (CMP, dGTP) | CMPK1 | 0.12 |
| cytidylate kinase (CMP, dGTP), nuclear | CMPK1 | 0.12 |
| cytidylate kinase (CMP, dGTP), nuclear | CMPK1 | 0.12 |
| cytidylate kinase (dCMP, dGTP) | CMPK1 | 0.12 |
| cytidylate kinase (dCMP, dGTP) | CMPK1 | 0.12 |
| cytidylate kinase (dCMP, dGTP), nuclear | CMPK1 | 0.12 |
| cytidylate kinase (dCMP, dGTP), nuclear | CMPK1 | 0.12 |
| cytidylate kinase (dCMP, dCTP) | CMPK1 | 0.12 |
| cytidylate kinase (dCMP, dCTP) | CMPK1 | 0.12 |
| cytidylate kinase (dCMP, dCTP), nuclear | CMPK1 | 0.12 |
| cytidylate kinase (dCMP, dCTP), nuclear | CMPK1 | 0.12 |
| cytidylate kinase (dCMP, dATP) | CMPK1 | 0.12 |
| cytidylate kinase (dCMP, dATP) | CMPK1 | 0.12 |
| cytidylate kinase (dCMP, dATP), nuclear | CMPK1 | 0.12 |
| cytidylate kinase (dCMP, dATP), nuclear | CMPK1 | 0.12 |
| cytidylate kinase (dCMP, UTP) | CMPK1 | 0.12 |
| cytidylate kinase (dCMP, UTP) | CMPK1 | 0.12 |
| cytidylate kinase (dCMP, CTP), nuclear | CMPK1 | 0.12 |
| cytidylate kinase (dCMP, CTP), nuclear | CMPK1 | 0.12 |
| cytidylate kinase (CMP), nuclear | CMPK1 | 0.12 |
| cytidylate kinase (CMP), nuclear | CMPK1 | 0.12 |
| cytidylate kinase (dCMP) | CMPK1 | 0.12 |
| cytidylate kinase (dCMP) | CMPK1 | 0.12 |
| cytidylate kinase (dCMP), nuclear | CMPK1 | 0.12 |
| cytidylate kinase (dCMP), nuclear | CMPK1 | 0.12 |
| cytidylate kinase (CMP)(GTP) | AK5 | 0.12 |
| cytidylate kinase (CMP)(GTP) | AK5 | 0.12 |
| cytidylate kinase (dCMP, CTP), nuclear | CMPK1 | 0.12 |
| cytidylate kinase (dCMP, CTP), nuclear | CMPK1 | 0.12 |
| cytidylate kinase (dCMP)(GTP) | AK5 | 0.12 |
| cytidylate kinase (dCMP)(GTP) | AK5 | 0.12 |
| cytidylate kinase (dCMP, GTP), nuclear | CMPK1 | 0.12 |
| cytidylate kinase (dCMP, GTP), nuclear | CMPK1 | 0.12 |
| cytidylate kinase (dCMP) | CMPK1 | 0.12 |
| cytidylate kinase (dCMP) | CMPK1 | 0.12 |
| cytidylate kinase (CMP), nuclear | CMPK1 | 0.12 |
| cytidylate kinase (CMP), nuclear | CMPK1 | 0.12 |
| cytidylate kinase (CMP, CTP) | CMPK1 | 0.12 |
| cytidylate kinase (CMP, CTP) | CMPK1 | 0.12 |
| cytidylate kinase (CMP, CTP), nuclear | CMPK1 | 0.12 |
| cytidylate kinase (CMP, CTP), nuclear | CMPK1 | 0.12 |
| cytidylate kinase (CMP, UTP) | CMPK1 | 0.12 |
| cytidylate kinase (CMP, UTP) | CMPK1 | 0.12 |
| cytidylate kinase (CMP, UTP), nuclear | CMPK1 | 0.12 |
| cytidylate kinase (CMP, UTP), nuclear | CMPK1 | 0.12 |
| cytidylate kinase (CMP, dATP) | CMPK1 | 0.12 |
| cytidylate kinase (CMP, dATP) | CMPK1 | 0.12 |
| cytidylate kinase (CMP, dATP), nuclear | CMPK1 | 0.12 |
| cytidylate kinase (CMP, dATP), nuclear | CMPK1 | 0.12 |
| cytidylate kinase (CMP, dCTP) | CMPK1 | 0.12 |
| cytidylate kinase (CMP, dCTP) | CMPK1 | 0.12 |
| cytidylate kinase (CMP, dCTP), nuclear | CMPK1 | 0.12 |
| cytidylate kinase (CMP, dCTP), nuclear | CMPK1 | 0.12 |
| nucleoside-diphosphate kinase (ATP: GDP), | GM20390; NME2 | 0.12 |
| nuclear | | |
| nucleoside-diphosphate kinase (ATP: GDP), | GM20390; NME2 | 0.12 |
| nuclear | | |
| nucleoside-diphosphate kinase | GM20390; NME2 | 0.12 |
| (ATP: CDP), nuclear | | |
| nucleoside-diphosphate kinase | GM20390; NME2 | 0.12 |
| (ATP: CDP), nuclear | | |
| nucleoside-diphosphate kinase (ATP: dGDP), | GM20390; NME2 | 0.12 |
| nuclear | | |
| nucleoside-diphosphate kinase (ATP: dGDP), | GM20390; NME2 | 0.12 |
| nuclear | | |
| nucleoside-diphosphate kinase (ATP: dCDP) | GM20390; | 0.12 |
| | NME2; NME3; | |
| | NME6; NME7 | |
| nucleoside-diphosphate kinase (ATP: dCDP), | GM20390; NME2 | 0.12 |
| nuclear | | |
| nucleoside-diphosphate kinase (ATP: dCDP), | GM20390; NME2 | 0.12 |
| nuclear | | |
| UMP kinase | CMPK1 | 0.12 |
| UMP kinase | CMPK1 | 0.12 |
| UMP kinase (CTP) | CMPK1 | 0.12 |
| UMP kinase (CTP) | CMPK1 | 0.12 |
| UMP kinase (CTP), nuclear | CMPK1 | 0.12 |
| UMP kinase (CTP), nuclear | CMPK1 | 0.12 |
| UMP kinase (UTP) | CMPK1 | 0.12 |
| UMP kinase (UTP) | CMPK1 | 0.12 |
| UMP kinase (UTP), nuclear | CMPK1 | 0.12 |
| UMP kinase (UTP), nuclear | CMPK1 | 0.12 |
| UMP kinase (GTP) | CMPK1 | 0.12 |
| UMP kinase (GTP) | CMPK1 | 0.12 |
| UMP kinase (GTP), nuclear | CMPK1 | 0.12 |
| UMP kinase (GTP), nuclear | CMPK1 | 0.12 |
| UMP kinase (dATP) | CMPK1 | 0.12 |
| UMP kinase (dATP) | CMPK1 | 0.12 |
| UMP kinase (dATP), nuclear | CMPK1 | 0.12 |
| UMP kinase (dATP), nuclear | CMPK1 | 0.12 |
| UMP kinase (dCTP) | CMPK1 | 0.12 |
| UMP kinase (dCTP) | CMPK1 | 0.12 |
| UMP kinase (dCTP), nuclear | CMPK1 | 0.12 |
| UMP kinase (dCTP), nuclear | CMPK1 | 0.12 |
| UMP kinase (dGTP) | CMPK1 | 0.12 |
| UMP kinase (dGTP) | CMPK1 | 0.12 |
| UMP kinase (dGTP), nuclear | CMPK1 | 0.12 |
| UMP kinase (dGTP), nuclear | CMPK1 | 0.12 |
| UMP kinase, nuclear | CMPK1 | 0.12 |
| UMP kinase, nuclear | CMPK1 | 0.12 |
| Nucleoside-diphosphate kinase (ATP: dGDP) | GM20390; | 0.12 |
| | NME2; NME3; | |
| | NME6; NME7 | |
| Nucleoside-diphosphate kinase (ATP: dADP) | GM20390; | 0.12 |
| | NME2; NME3; | |
| | NME6; NME7 | |
| Resistance-Nodulation-Cell Division (RND) | SLCO1A1 | 0.12 |
| TCDB: 2.A.60.1.14 | | |
| glucosamine-6-phosphate deaminase | GNPDA1; | 0.12 |
| | GNPDA2 | |
| CO2 transport (diffusion), mitochondrial | | 0.12 |
| hypothiocyanite exchange | | 0.11 |
| RE0702 | LPO | 0.11 |
| esterification of hexanoylcoa for transport into | CROT | 0.11 |
| cytosol | | |
| transport of hexanoylcarnitine into cytosol | SLC25A20 | 0.11 |
| fatty acid beta oxidation(C8-->C6)x | ACAA1B; ACOX1; | 0.11 |
| | EHHADH; | |
| | HSD17B4 | |
| Carbonyl reductase [NADPH] 1 | CBR1 | 0.11 |
| inositol 1,4-bisphosphate nuclear transport | | 0.11 |
| (diffusion) | | |
| phosphatidylinositol 4-phosphate phospholipase | PLCB1 | 0.11 |
| C, nucleus | | |
| Major Facilitator(MFS) TCDB: 2.A.1.44.1 | SLC43A1 | 0.11 |
| Ethanolamine kinase | CHKA; CHKB; | 0.11 |
| | ETNK1; ETNK2 | |
| ethanolamine phosphate phosphatase | PHOSPHO1 | 0.11 |
| Vesicular transport | | 0.11 |
| bile acid intracellular transport | | 0.11 |
| RE2626 | CYP27A1 | 0.11 |
| RE3346 | ALDH1B1; | 0.11 |
| | ALDH2; | |
| | ALDH7A1 | |
| D-fructose transport in via uniport | SLC2A2; | 0.11 |
| | SLC2A5; | |
| | SLC2A7; SLC2A8 | |
| D-fructose transport via sodium cotransport | SLC5A10; | 0.11 |
| | SLC5A9 | |
| fatty-acid--CoA ligase | ACSBG2; ACSL1; | 0.11 |
| | ACSL3; ACSL4 | |
| uptake of linoleic acid by the enterocytes | SLC27A4 | 0.11 |
| Linoleic acid (n-C18: 2) transport in via diffusion | SLC27A5 | 0.11 |
| Vitamin D3 transport from mitochondria | | 0.11 |
| Na+/iodide cotransport | SLC5A5 | 0.11 |
| glutamine-fructose-6-phosphate transaminase | GFPT1; GFPT2 | 0.11 |
| glutamine synthetase | GLUL; LGSN | 0.11 |
| Farnesyl-diphosphate: farnesyl-diphosphate | FDFT1 | 0.11 |
| farnesyltransferase Biosynthesis of steroids | | |
| EC: 2.5.1.21 | | |
| Presqualene diphosphate: farnesyl-diphosphate | FDFT1 | 0.11 |
| farnesyltransferase Biosynthesis of steroids | | |
| EC: 2.5.1.21 | | |
| Squalene synthase | FDFT1 | 0.11 |
| Biotin transport via sodium symport | SLC5A6 | 0.11 |
| Biotin transport via sodium symport | SLC5A6 | 0.11 |
| phosphate transport in/out via three Na+ | SLC17A1; | 0.11 |
| symporter | SLC17A2; | |
| | SLC17A3; | |
| | SLC17A4; | |
| | SLC34A1; | |
| | SLC34A2 | |
| L-Phenylalanine, tetrahydrobiopterin: oxygen | PAH | 0.11 |
| oxidoreductase (4-hydroxylating) Phenylalanine, | | |
| tyrosine and tryptophan biosynthesis | | |
| EC: 1.14.16.1 | | |
| galactose-1-phosphate uridylyltransferase | GALT | 0.11 |
| galactose-1-phosphate uridylyltransferase | GALT | 0.11 |
| UTP-glucose-1-phosphate uridylyltransferase | UGP2 | 0.11 |
| UTP-glucose-1-phosphate uridylyltransferase | UGP2 | 0.11 |
| UDPglucose--hexose-1-phosphate | GALT | 0.11 |
| uridylyItransferase | | |
| UDPglucose--hexose-1-phosphate | GALT | 0.11 |
| uridylyItransferase | | |
| bicarbonate transport (Na/HCO3 cotransport) | SLC4A7 | 0.11 |
| 3HCO3 NAt | SLC4A4; SLC4A5 | 0.11 |
| Zinc (Zn2+)-Iron (Fe2+) Permease (ZIP), | SLC26A1 | 0.11 |
| TCDB: 2.A.53.2.2 | | |
| ATP-binding Cassette (ABC) TCDB: 3.A.1.208.15 | ABCC5 | 0.11 |
| D-glucurono-6,3-lactone transport, endoplasmic | | 0.11 |
| reticulum | | |
| glucuronate endoplasmic reticular transport | | 0.11 |
| gluconolactonase, endoplasmic reticulum | | 0.11 |
| glucuronolactone reductase | | 0.11 |
| gulonolactone endoplasmic reticular transport | | 0.11 |
| gulonate dehydrogenase, endoplasmic reticulum | | 0.11 |
| L-gulonate endoplasmic reticular export | | 0.11 |
| RE0383 | AKR1A1 | 0.11 |
| uronolactonase, endoplasmic reticulum | | 0.11 |
| exchange reaction for pan4p | | 0.11 |
| Amino Acid-Polyamine-Organocation (APC) | SLC7A9 | 0.11 |
| TCDB: 2.A.3.8.15 | | |
| Amino Acid-Polyamine-Organocation (APC) | SLC7A9 | 0.11 |
| TCDB: 2.A.3.8.15 | | |
| Amino Acid-Polyamine-Organocation (APC) | SLC7A9 | 0.11 |
| TCDB: 2.A.3.8.15 | | |
| L-cystine/L-glutamate exchanger | SLC3A2; | 0.11 |
| | SLC7A11 | |
| cytidine facilated transport in cytosol | SLC29A1; | 0.11 |
| | SLC29A2 | |
| cytidine transport in via sodium symport | SLC28A1; | 0.11 |
| | SLC28A3 | |
| adenosine transport (1:2 Na/Adn cotransport) | SLC28A3 | 0.11 |
| cytidine transport (1:2 Na/cytd cotransport) | SLC28A3 | 0.11 |
| guanosine transport in via sodium (1:2) symport | SLC28A3 | 0.11 |
| inosine transport in via sodium (1:2) symport | SLC28A3 | 0.11 |
| uridine transport in via sodium symport (1:2) | SLC28A3 | 0.11 |
| ATP diffusion in nucleus | | 0.11 |
| NAD transport, nuclear through pores | | 0.11 |
| nicotinamide-nucleotide adenylyltransferase | NMNAT1 | 0.11 |
| NMN transport, nuclear trhough pore | | 0.11 |
| Cytochrome P450 27 | CYP27A1 | 0.11 |
| bicarbonate transport (Cl—/HCO3— exchange) | SLC4A1; | 0.11 |
| | SLC4A2; | |
| | SLC4A3; SLC4A9 | |
| choline phosphotransferase | CEPT1; CHPT1 | 0.11 |
| choline phosphate cytididyltransferase | PCYT1A; | 0.11 |
| | PCYT1B | |
| glyoxylate transport, mitochondrial | | 0.10 |
| bicarbonate transport (Na/HCO3 1:2 | SLC4A4; SLC4A5 | 0.10 |
| cotransport) | | |
| Resistance-Nodulation-Cell Division (RND) | CP | 0.10 |
| TCDB: 2.A.65.1.1 | | |
| bicarbonate transport (Na/HCO3 1:2 | SLC4A4; SLC4A5 | 0.10 |
| cotransport) | | |
| prostaglandine E2 transport (ATP-dependent) | ABCC4 | 0.10 |
| Triiodothyronine sulfate exchange | | 0.10 |
| Triiodothyronine sulfate transport (diffusion) | | 0.10 |
| Triiodothyronine Sulfotransferase | SULT1A1 | 0.10 |
| L-alanine/L-glutamine Na-dependent exchange | SLC1A5 | 0.10 |
| (Ala-L in) | | |
| L-cysteine/L-glutamine Na-dependent exchange | SLC1A5 | 0.10 |
| (Cys-L in) | | |
| L-alanine/L-glutamine Na-dependent exchange | SLC1A5 | 0.10 |
| (Gln-L in) | | |
| L-cysteine/L-glutamine Na-dependent exchange | SLC1A5 | 0.10 |
| (Gln-L in) | | |
| L-serine/L-glutamine Na-dependent exchange | SLC1A5 | 0.10 |
| (Gln-L in) | | |
| L-threonine/L-glutamine Na-dependent | SLC1A5 | 0.10 |
| exchange (Gln-L in) | | |
| L-serine/L-glutamine Na-dependent exchange | SLC1A5 | 0.10 |
| (Ser-L in) | | |
| L-threonine/L-glutamine Na-dependent | SLC1A5 | 0.10 |
| exchange (Thr-L in) | | |
| Prostaglandin E2 exchange | | 0.10 |
| RE2069 | | 0.10 |
| L-Serine hydro-lyase (adding homocysteine) | CBS | 0.10 |
| Glycine, serine and threonine metabolism | | |
| EC: 4.2.1.22 | | |
| L-Glutamate exchange | | 0.10 |
| chloride transport via hydroxide | SLC26A6 | 0.10 |
| countertransport (2:1) | | |
| Resistance-Nodulation-Cell Division (RND) | SLCO1A1 | 0.10 |
| TCDB: 2.A.60.1.14 | | |
| Major Facilitator(MFS) TCDB: 2.A.1.14.6 | SLC17A1 | 0.10 |
| Iodide uniport | SLC5A8 | 0.10 |
| transport of beta alanine into the intestinal cells | SLC6A6 | 0.10 |
| by beta transport system | | |
| Amino acid transporter ATB0+ Facilitated | | 0.10 |
| diffusion | | |
| inositol transport via sodium symport | SLC5A3 | 0.10 |
| inositol transport via sodium symport | SLC5A11 | 0.10 |
| sodium transport (uniport) | SLC5A1; | 0.10 |
| | SLC5A3; SLC5A5 | |
| EC: 1.1.1.42 | IDH1; IDH2; | 0.10 |
| | IDH3A; IDH3B; | |
| | IDH3G | |
| chloride transport via iodide countertransport | SLC26A4 | 0.10 |
| Phosphatidylserine synthase homo sapiens | PTDSS1 | 0.10 |
| nucleoside-diphosphate kinase (ATP: UDP), | GM20390; NME2 | 0.10 |
| nuclear | | |
| nucleoside-diphosphate kinase (ATP: UDP), | GM20390; NME2 | 0.10 |
| nuclear | | |
| exchange reaction for L-glutamine | | 0.10 |
| NAD transporter, peroxisome | | 0.10 |
| Resistance-Nodulation-Cell Division (RND) | SLCO1A1 | 0.10 |
| TCDB: 2.A.60.1.14 | | |
| Isocitrate: NADP+ oxidoreductase | IDH1; IDH2 | 0.10 |
| (decarboxylating) Citrate cycle (TCA cycle) | | |
| EC: 1.1.1.42 | | |
| androsterone transport | | 0.10 |
| androsterone intracellular transport | | 0.10 |
| Androsterone exchange | | 0.10 |
| Creatine transport (sodium symport) (2:1) | SLC6A8 | 0.10 |
| bile acid intracellular transport | | 0.10 |
| Iodide uniport | SLC5A8 | 0.10 |
| Norepinephrine reversible transport in via | SLC6A2; SLC6A3 | 0.10 |
| sodium symport (1:2) | | |
| Norepinephrine uniport | SLC22A1; | 0.10 |
| | SLC22A2; | |
| | SLC22A3 | |
| Neurotransmitter: Sodium Symporter (NSS) | SLC6A8 | 0.10 |
| TCDB: 2.A.22.3.4 | | |
| Glycoside-Pentoside-Hexuronide (GPH): Cation | SLC10A1 | 0.10 |
| Symporter TCDB: 2.A.28.1.1 | | |
| Glycoside-Pentoside-Hexuronide (GPH): Cation | SLC10A1 | 0.10 |
| Symporter TCDB: 2.A.28.1.1 | | |
| bile acid intracellular transport | | 0.10 |
| citrate transport via sodium symport | SLC13A5 | 0.10 |
| L-Cystine exchange | | 0.10 |
| Pantothenate sodium symporter II | SLC5A6 | 0.10 |
| Deoxycytidine kinase, nuclear (ATP) | DCK | 0.10 |
| Deoxycytidine kinase, nuclear (ATP) | DCK | 0.10 |
| Deoxycytidine kinase, nuclear (UTP) | DCK | 0.10 |
| Deoxycytidine kinase, nuclear (UTP) | DCK | 0.10 |
| ATP exchange | | 0.10 |
| acetyl-CoA C-acetyltransferase, mitochondrial | ACAT3 | 0.10 |
| (S)-3-Hydroxy-3-methylglutaryl-CoA acetoacetyl- | HMGCS1 | 0.10 |
| CoA-lyase (CoA-acetylating) Synthesis and | | |
| degradation of ketone bodies/Valine, leucine | | |
| and isoleucine degradation/Butanoate | | |
| metabolism EC: 2.3.3.10 | | |
| galactose transport (uniport) | SLC2A1; | 0.10 |
| | SLC2A10; | |
| | SLC2A2; | |
| | SLC2A3; SLC2A8 | |
| galactose transport via sodium symport | SLC5A10; | 0.10 |
| | SLC5A2; SLC5A9 | |
| mannose transport (uniport) | SLC2A1; | 0.10 |
| | SLC2A2; SLC2A3 | |
| D-mannose transport via sodium cotransport | SLC5A10; | 0.10 |
| | SLC5A9 | |
| phosphoglucomutase | PGM1; PGM2 | 0.10 |
| 2-Methylpropanoyl-CoA: oxygen 2,3- | ACADS | 0.10 |
| oxidoreductase Valine, leucine and isoleucine | | |
| degradation EC: 1.3.99.2 | | |
| fatty acid intracellular transport | | 0.10 |
| Octanoyl-CoA: L-carnitine O-octanoyltransferase | CROT | 0.10 |
| EC: 2.3.1.137 | | |
| Octanoyl-CoA: L-carnitine O-octanoyltransferase | CROT | 0.10 |
| EC: 2.3.1.137 | | |
| COT Facilitated diffusion | CROT | 0.10 |
| Mitochondrial Carrier (MC) TCDB: 2.A.29.8.3 | SLC25A20 | 0.10 |
| transport of bytyrylcarnitine from peroxisomes | SLC25A20 | 0.09 |
| esterification of butyrylcoa to butyrylcarnitine | CROT | 0.09 |
| for transport | | |
| esterification of hexanoylcoa for transport into | CROT | 0.09 |
| cytosol | | |
| transport of hexanoylcarnitine into cytosol | SLC25A20 | 0.09 |
| fatty acid beta oxidation(C6-->C4)x | ACAA1B; ACOX1; | 0.09 |
| | EHHADH; | |
| | HSD17B4 | |
| taurine transport (sodium symport) (2:1) | SLC6A6 | 0.09 |
| galactokinase | GALK1; GALK2 | 0.09 |
| UDPglucose 4-epimerase | GALE | 0.09 |
| fatty acid transport via diffusion | | 0.09 |
| fatty acid transport via diffusion | SLC27A5 | 0.09 |
| fatty acid transport via diffusion | | 0.09 |
| fatty acid transport via diffusion | | 0.09 |
| fatty acid electroneutral transport | SLC27A5 | 0.09 |
| fatty acid electroneutral transport | SLC27A5 | 0.09 |
| fatty acid electroneutral transport | SLC27A2; | 0.09 |
| | SLC27A5 | |
| fatty acid electroneutral transport | SLC27A2; | 0.09 |
| | SLC27A5 | |
| fatty acid electroneutral transport | SLC27A2; | 0.09 |
| | SLC27A5 | |
| fatty acid electroneutral transport | SLC27A2; | 0.09 |
| | SLC27A5 | |
| fatty acid transport via diffusion | SLC27A2 | 0.09 |
| fatty acid transport via diffusion | | 0.09 |
| Transport of (R)-Pantothenate | | 0.09 |
| Active transport | | 0.09 |
| Major Facilitator(MFS) TCDB: 2.A.18.6.3 | SLC38A3 | 0.09 |
| Major Facilitator(MFS) TCDB: 2.A.18.6.3 | SLC38A3 | 0.09 |
| fatty acid electroneutral transport | SLC27A1 | 0.09 |
| fatty acid electroneutral transport | SLC27A1 | 0.09 |
| fatty acid transport via diffusion | SLC27A5 | 0.09 |
| fatty acid transport via diffusion | SLC27A5 | 0.09 |
| fatty acid electroneutral transport | SLC27A1; | 0.09 |
| | SLC27A3; | |
| | SLC27A4 | |
| fatty acid transport via diffusion | SLC27A5 | 0.09 |
| Amino Acid-Polyamine-Organocation (APC) | SLC7A9 | 0.09 |
| TCDB: 2.A.3.8.15 | | |
| Amino Acid-Polyamine-Organocation (APC) | SLC7A9 | 0.09 |
| TCDB: 2.A.3.8.15 | | |
| Transport of 3-methyl-2-oxopentanoate | | 0.09 |
| Exchange of 3-methyl-2-oxopentanoate | | 0.09 |
| sodium transport (uniport) | SLC5A1; | 0.09 |
| | SLC5A3; SLC5A5 | |
| glucose transport (uniport) | SLC2A1; | 0.09 |
| | SLC2A10; | |
| | SLC2A12; | |
| | SLC2A2; | |
| | SLC2A3; | |
| | SLC2A4; | |
| | SLC2A6; | |
| | SLC2A7; | |
| | SLC2A8; SLC2A9 | |
| glucose transport via sodium symport | SLC5A10; | 0.09 |
| | SLC5A11; | |
| | SLC5A12; | |
| | SLC5A2; | |
| | SLC5A3; SLC5A9 | |
| exchange reaction for D-Glucosamine | | 0.09 |
| glucosamine transport (uniport) | SLC2A1; | 0.09 |
| | SLC2A2; SLC2A4 | |
| hexokinase (D-glucosamine: ATP) | GCK; HK1; HK2; | 0.09 |
| | HK3; HKDC1 | |
| Glycolysis/Glyconeogenesis EC: 2.7.1.1 | HK1; HK2; HK3 | 0.09 |
| Glycolysis/Glyconeogenesis EC: 2.7.1.1 | HK1; HK2; HK3 | 0.09 |
| myo-Inositol exchange | | 0.09 |
| inositol 1-phosphate nuclear transport | | 0.09 |
| (diffusion) | | |
| phosphatidylinositol phospholipase C, nucleus | PLCB1 | 0.09 |
| inositol transport in via proton symport | SLC2A13 | 0.09 |
| transport of L-Cystine into the cell in exchange | SLC3A1; SLC7A9 | 0.09 |
| for L-Leucine by b0,+AT transporter at the apical | | |
| surfaces of the membranes of small intestine and | | |
| renal cells. | | |
| release of heme into the blood | MFSD7B; | 0.09 |
| | SLC46A1 | |
| heme transport | LRP1; SLC46A1 | 0.09 |
| Na+—K+—Cl— cotransport (NH4+) | SLC12A1; | 0.09 |
| | SLC12A2 | |
| bilirubin beta-diglucuronide transport MDR | ABCC1; ABCC2; | 0.09 |
| | ABCC3 | |
| bilirubin monoglucuronide transport MDR | ABCC1 | 0.09 |
| 3-hydroxyisobutyryl-CoA hydrolase, | | 0.09 |
| mitochondrial | | |
| 3-hydroxyacyl-CoA dehydratase (3- | ECHS1; HADHA; | 0.09 |
| hydroxyisobutyryl-CoA) (mitochondria) | HADHB | |
| L-Cysteine L-homocysteine-lyase (deaminating) | CTH | 0.09 |
| Cysteine metabolism EC: 4.4.1.1 | | |
| 3-Mercaptopyruvate: cyanide sulfurtransferase | MPST; TST | 0.09 |
| Cysteine metabolism EC: 2.8.1.2 | | |
| Spontaneous reaction | | 0.09 |
| H2O transport, nuclear | | 0.09 |
| phosphatidylinositol 4,5-bisphosphate nuclear | | 0.09 |
| transport (diffusion) | | |
| phosphate transport, nuclear | | 0.09 |
| L-Methionine exchange | | 0.09 |
| exchange reaction for D-Mannose | | 0.09 |
| hexokinase (D-mannose: ATP) | GCK; HK1; HK2; | 0.09 |
| | HK3; HKDC1 | |
| mannose-6-phosphate isomerase | MPI | 0.09 |
| Glycolysis/Glyconeogenesis EC: 2.7.1.1 | HK1; HK2; HK3 | 0.09 |
| Glycolysis/Glyconeogenesis EC: 2.7.1.1 | HK1; HK2; HK3 | 0.09 |
| glucose transport (uniport) | SLC2A1; | 0.09 |
| | SLC2A10; | |
| | SLC2A12; | |
| | SLC2A2; | |
| | SLC2A3; | |
| | SLC2A4; | |
| | SLC2A6; | |
| | SLC2A7; | |
| | SLC2A8; SLC2A9 | |
| glucose transport via sodium symport | SLC5A10; | 0.09 |
| | SLC5A11; | |
| | SLC5A12; | |
| | SLC5A2; | |
| | SLC5A3; SLC5A9 | |
| Octanoyl-CoA transport, diffusion | | 0.09 |
| exchange reaction for L-asparagine | | 0.09 |
| Amino Acid-Polyamine-Organocation (APC) | SLC7A9 | 0.09 |
| TCDB: 2.A.3.8.15 | | |
| Amino Acid-Polyamine-Organocation (APC) | SLC7A9 | 0.09 |
| TCDB: 2.A.3.8.15 | | |
| Amino Acid-Polyamine-Organocation (APC) | SLC7A9 | 0.09 |
| TCDB: 2.A.3.8.15 | | |
| Amino Acid-Polyamine-Organocation (APC) | SLC7A9 | 0.09 |
| TCDB: 2.A.3.8.15 | | |
| Amino Acid-Polyamine-Organocation (APC) | SLC7A9 | 0.09 |
| TCDB: 2.A.3.8.15 | | |
| Amino Acid-Polyamine-Organocation (APC) | SLC7A9 | 0.09 |
| TCDB: 2.A.3.8.15 | | |
| Amino Acid-Polyamine-Organocation (APC) | SLC7A9 | 0.09 |
| TCDB: 2.A.3.8.15 | | |
| production of decenoylcarnitine | CPT1A; CPT1B; | 0.09 |
| | CPT1C | |
| transport of decenoyl carnitine into extra cellular | | 0.09 |
| space | | |
| production of decadienoylcarnitine | CPT1A; CPT1B; | 0.09 |
| | CPT1C | |
| production of dodecenoylcarnitine | CPT1A; CPT1B; | 0.09 |
| | CPT1C | |
| production of tetradecenoylcarnitine | CPT1A; CPT1B; | 0.09 |
| | CPT1C | |
| production of tetradecadienoylcarnitine | CPT1A; CPT1B; | 0.09 |
| | CPT1C | |
| production of ocetenoylcarnitine | CPT1A; CPT1B; | 0.09 |
| | CPT1C | |
| transport of octenoyl carnitine into the extra | | 0.09 |
| cellular space | | |
| transport of dodecenoyl carnitine into extra | | 0.09 |
| cellular space | | |
| transport of decadienoyl carnitine into the extra | | 0.09 |
| cellular space | | |
| exchange reaction for decenoyl carnitine | | 0.09 |
| exchange reaction for octenoyl carnitine | | 0.09 |
| exchange reaction for dodecenoyl carnitine | | 0.09 |
| exchange reaction for decadienoyl carnitine | | 0.09 |
| exchange reaction for tetradecadienoyl carnitine | | 0.09 |
| exchange reaction for tetradecenoyl carnitine | | 0.09 |
| C101COASINK | | 0.09 |
| C81COASINK | | 0.09 |
| DD2COASINK | | 0.09 |
| Sink for 2,6-dodecadienoylcoa(c) | | 0.09 |
| TETDEC2COASINK | | 0.09 |
| TETDECE1COASINK | | 0.09 |
| transport of tetradecadienoyl carnitine into extra | | 0.09 |
| cellular space | | |
| transport of tetradecenoyl carnitine into extra | | 0.09 |
| cellular space | | |
| Pantothenate sodium symporter II | SLC5A6 | 0.09 |
| D-aspartate transport via Na, H symport and K | SLC1A1; | 0.09 |
| antiport | SLC1A2; | |
| | SLC1A3; | |
| | SLC1A6; SLC1A7 | |
| D-aspartate transport, extracellular | | 0.09 |
| L-aspartate transport via Na, H symport and K | SLC1A1; | 0.09 |
| antiport | SLC1A2; | |
| | SLC1A3; | |
| | SLC1A6; SLC1A7 | |
| Glutamate transport via Na, H symport and K | SLC1A1; | 0.09 |
| antiport | SLC1A2; | |
| | SLC1A3; | |
| | SLC1A6; SLC1A7 | |
| hexokinase (D-glucose: ATP) | GCK; HK1; HK2; | 0.09 |
| | HK3; HKDC1 | |
| Glycolysis/Glyconeogenesis EC: 2.7.1.1 | GCK; HK1; HK2; | 0.09 |
| | HK3 | |
| Glycolysis/Glyconeogenesis EC: 2.7.1.1 | GCK; HK1; HK2; | 0.09 |
| | HK3 | |
| phosphate transport in/out via two Na+ | SLC34A3 | 0.09 |
| symporter | | |
| Major Facilitator(MFS) TCDB: 2.A.1.14.6 | SLC17A1 | 0.09 |
| transport of Alanine by y+LAT1 or y+LAT2 with | SLC3A2; | 0.09 |
| co-transporter of h in small intestine and kidney | SLC7A6; SLC7A7 | |
| L-alanine reversible transport via proton | SLC36A1; | 0.09 |
| symport | SLC36A2 | |
| transport of acetylcarnitine from peroxisomes to | SLC25A20 | 0.09 |
| cytosol | | |
| inositol transport via sodium symport | SLC5A3 | 0.09 |
| inositol transport via sodium symport | SLC5A11 | 0.09 |
| Na+—K+—Cl— cotransport | SLC12A1; | 0.09 |
| | SLC12A2 | |
| Utilized transport | | 0.09 |
| Utilized transport | | 0.09 |
| UDPGlcA endoplasmic reticulum transport via | SLC35D1 | 0.09 |
| UMP antiport | | |
| UDPGlcA endoplasmic reticulum transport via | SLC35D1 | 0.09 |
| UMP antiport | | |
| UMP transport (ER) | | 0.09 |
| UMP transport (ER) | | 0.09 |
| L-carnitine reversible transport | SLC22A5 | 0.09 |
| L-carnitine outward transport (H+ antiport) | SLC22A4 | 0.09 |
| acid phosphatase (FMN) | ACP1; ACP5; | 0.09 |
| | ACP6; ACPP | |
| riboflavin kinase | RFK | 0.09 |
| transport of L-Cystine into the cell in exchange | SLC3A1; SLC7A9 | 0.09 |
| for L-Alanine by b0,+AT transporter at the apical | | |
| surfaces of the membranes of small intestine and | | |
| renal cells. | | |
| Amino Acid-Polyamine-Organocation (APC) | SLC7A9 | 0.09 |
| TCDB: 2.A.3.8.15 | | |
| Transport of L-Histidine by y+LAT1 or y+LAT2 | SLC3A2; | 0.09 |
| transporters in small intestine and kidney | SLC7A6; SLC7A7 | |
| Histamine exchange | | 0.09 |
| Dopachrome tautomerase | DCT | 0.09 |
| DM melanin(c) | | 0.09 |
| Dopaquinone isomerase 1 | | 0.09 |
| RE3198 | | 0.09 |
| Tyrosinase | TYRP1 | 0.09 |
| Tyrosine: dopa oxidase (dopaquinone producing | TYR | 0.09 |
| 2) | | |
| inositol transport in via proton symport | SLC2A13 | 0.08 |
| Isocitrate dehydrogenase (NADP+) | IDH1 | 0.08 |
| retinoic acid transport | | 0.08 |
| Retinoate exchange | | 0.08 |
| NAD synthase (glutamine-hydrolysing) | NADSYN1 | 0.08 |
| nicotinamide-nucleotide adenylyltransferase | NMNAT2; | 0.08 |
| | NMNAT3 | |
| Nicotinamide-D-ribonucleotide amidohydrolase | | 0.08 |
| Nicotinate and nicotinamide metabolism | | |
| EC: 3.5.1.42 | | |
| Deamino-NAD+ nucleotidohydrolase Nicotinate | ENPP1; ENPP3; | 0.08 |
| and nicotinamide metabolism EC: 3.6.1.9 | NUDT12 | |
| binding of betaglucans with taurocholate in the | | 0.08 |
| intestinal lumen, reducing serum cholesterol | | |
| levels. | | |
| exchange reaction for beta glucan-taurocholic | | 0.08 |
| acid complex | | |
| exchange reaction for guar gum-taurocholic acid | | 0.08 |
| complex | | |
| exchange reaction for psyllium-taurocholic acid | | 0.08 |
| complex | | |
| Taurocholic acid exchange | | 0.08 |
| Binding of guar gums with taurocholate in the | | 0.08 |
| intestinal lumen, reducing serum cholesterol | | |
| levels. | | |
| binding of psyllium with taurocholate in the | | 0.08 |
| intestinal lumen, reducing serum cholesterol | | |
| levels. | | |
| Choloyl-CoA: glycine N-choloyltransferase Bile | BAAT | 0.08 |
| acid biosynthesis/Taurine and hypotaurine | | |
| metabolism EC: 2.3.1.65 | | |
| cysteine transaminase | GOT1 | 0.08 |
| carnitine O-acetyltransferase, reverse direction, | CRAT | 0.08 |
| peroxisomal | | |
| transport of butyryl carnitine into the extra | | 0.08 |
| cellular space | | |
| exchange reaction for butyryl carnitine | | 0.08 |
| Phosphate exchange | | 0.08 |
| L-cystine/glycine exchanger (cystine in) | SLC3A1; SLC7A9 | 0.08 |
| (S)-2-methylbutanoyl-CoA: enzyme N6- | DBT | 0.08 |
| (dihydrolipoyl)lysine S-(2- | | |
| methylbutanoyl)transferase Valine, leucine and | | |
| isoleucine degradation EC: 2.3.1.168 | | |
| (S)-3-Methyl-2- | BCKDHA; | 0.08 |
| oxopentanoate: [dihydrolipoyllysine-residue (2- | BCKDHB; | |
| methylpropanoyl)transferase] lipoyllysine 2- | TMEM91 | |
| oxidoreductase (decarboxylating, acceptor-2- | | |
| methylpropanoylating) EC: 1.2.4.4 | | |
| Nicotinate exchange | | 0.08 |
| 3-sn-phosphatidate phosphohydrolase | PLPP1; PLPP2; | 0.08 |
| Sphingolipid metabolism EC: 3.1.3.4 | PLPP3; SGPP1; | |
| | SGPP2 | |
| sphingolipid long chain base kinase | SPHK1; SPHK2 | 0.08 |
| (sphinganine) | | |
| RE2660 | | 0.08 |
| RE2659 | | 0.08 |
| production of 3-OHdecanoylcarnitinec | CPT1A; CPT1B; | 0.08 |
| | CPT1C | |
| transport of 3-hydroxydecanoyl carnitine into | | 0.08 |
| extra cellular space | | |
| exchange reaction for 3-hydroxydecanoyl | | 0.08 |
| carnitine | | |
| fatty acid beta oxidation(C10-->OHC10)m | ACADM; ECHS1 | 0.08 |
| transport of (S)-3-Hydroxydecanoyl-CoA from | DBI | 0.08 |
| mitochondria into the cytosol | | |
| bicarbonate transport (HCl/NaHCO3 exchange) | SLC4A10; | 0.08 |
| | SLC4A8 | |
| dUTP transport via dTDP antiport | SLC25A19 | 0.08 |
| dUTP transport via dGDP antiport | SLC25A19 | 0.08 |
| dUTP transport via dADP antiport | SLC25A19 | 0.08 |
| dUTP transport via dCDP antiport | SLC25A19 | 0.08 |
| Major Facilitator(MFS) TCDB: 2.A.1.44.1 | SLC43A1 | 0.08 |
| Major Facilitator(MFS) TCDB: 2.A.1.44.1 | SLC43A1 | 0.08 |
| Dopamine reversible transport in via sodium | SLC6A2; SLC6A3 | 0.08 |
| symport (1:2) | | |
| Dopamine uniport | SLC22A2; | 0.08 |
| | SLC22A3; | |
| | SLC22A5 | |
| choline, sodium cotransport | SLC5A7 | 0.08 |
| Choline uniport | SLC22A2; | 0.08 |
| | SLC22A5 | |
| mannose-1-phosphate guanylyltransferase (GDP) | GMPPA; GMPPB | 0.08 |
| GTP: alpha-D-mannose-1-phosphate | GMPPA; GMPPB | 0.08 |
| guanylyltransferase Fructose and mannose | | |
| metabolism EC: 2.7.7.13 | | |
| 3-methyl-2-oxobutanoate: [dihydrolipoyllysine- | BCKDHA; | 0.08 |
| residue (2-methylpropanoyl)transferase] | BCKDHB; | |
| lipoyllysine 2-oxidoreductase (decarboxylating, | TMEM91 | |
| acceptor-2-methylpropanoylating) EC: 1.2.4.4 | | |
| demand reaction for taurine | | 0.08 |
| exit of taurine from the enterocytes | | 0.08 |
| Na+—Cl— cotransport | SLC12A3 | 0.08 |
| Utilized transport | | 0.08 |
| Na+—K+—Cl— cotransport (NH4+) | SLC12A1; | 0.08 |
| | SLC12A2 | |
| Triiodothyronine exchange | | 0.08 |
| AKG transport via sodium symport | SLC13A3 | 0.08 |
| (S)-Methylmalonyl-CoA hydrolase Propanoate | | 0.08 |
| metabolism EC: 3.1.2.17 | | |
| (S)-Methylmalonate semialdehyde: NAD+ | ALDH1B1; | 0.08 |
| oxidoreductase Valine, leucine and isoleucine | ALDH2; | |
| degradation EC: 1.2.1.3 | ALDH3A2; | |
| | ALDH7A1; | |
| | ALDH9A1 | |
| Pyruvate exchange | | 0.08 |
| Oxalosuccinate: NADP+ oxidoreductase | IDH1; IDH2; | 0.08 |
| (decarboxylating) Citrate cycle (TCA cycle) | IDH3A; IDH3B; | |
| EC: 1.1.1.42 | IDH3G | |
| Isocitrate dehydrogenase (NADP+) | IDH2 | 0.08 |
| N1-Methylnicotinamide transport | | 0.07 |
| 1-Methylnicotinamide exchange | | 0.07 |
| Nicotinamide N-methyltransferase | NNMT | 0.07 |
| Exchange of pyridoxal 5-phosphate(2-) | | 0.07 |
| Diffusion | | 0.07 |
| intracellular transport | | 0.07 |
| phosphatidate cytidylyltransferase | CDS1; CDS2 | 0.07 |
| choline transport via diffusion (cytosol to | | 0.07 |
| mitochondria) | | |
| cytidine kinase (ATP), mitochondrial | | 0.07 |
| cytidine facilated transport in mitochondria | SLC29A1 | 0.07 |
| cytidylate kinase (CMP), mitochondrial | CMPK2 | 0.07 |
| nucleoside-diphosphate kinase (ATP: CDP), | NME4; NME6 | 0.07 |
| mitochondrial | | |
| choline phosphatase | PLD1 | 0.07 |
| L-alanine/L-cysteine Na-dependent exchange | SLC1A4; SLC1A5 | 0.07 |
| (Ala-L in) | | |
| L-alanine/L-serine Na-dependent exchange (Ala- | SLC1A4; SLC1A5 | 0.07 |
| L in) | | |
| L-alanine/L-threonine Na-dependent exchange | SLC1A4; SLC1A5 | 0.07 |
| (Ala-L in) | | |
| L-alanine/L-cysteine Na-dependent exchange | SLC1A4; SLC1A5 | 0.07 |
| (Cys-L in) | | |
| L-serine/L-cysteine Na-dependent exchange | SLC1A4; SLC1A5 | 0.07 |
| (Cys-L in) | | |
| L-cysteine/L-threonine Na-dependent exchange | SLC1A4; SLC1A5 | 0.07 |
| (Cys-L in) | | |
| L-alanine/L-serine Na-dependent exchange (Ser- | SLC1A4; SLC1A5 | 0.07 |
| L in) | | |
| L-serine/L-cysteine Na-dependent exchange | SLC1A4; SLC1A5 | 0.07 |
| (Ser-L in) | | |
| L-serine/L-threonine Na-dependent exchange | SLC1A4; SLC1A5 | 0.07 |
| (Ser-L in) | | |
| L-alanine/L-threonine Na-dependent exchange | SLC1A4; SLC1A5 | 0.07 |
| (Thr-L in) | | |
| L-cysteine/L-threonine Na-dependent exchange | SLC1A4; SLC1A5 | 0.07 |
| (Thr-L in) | | |
| L-serine/L-threonine Na-dependent exchange | SLC1A4; SLC1A5 | 0.07 |
| (Thr-L in) | | |
| cysteine oxidase | CDO1 | 0.07 |
| L-Threonine exchange | | 0.07 |
| L-threonine deaminase | SDS; SDSL | 0.07 |
| 3,4-Dihydroxy-L-phenylalanine exchange | | 0.07 |
| choline intracellular transport | | 0.07 |
| choline intracellular transport | | 0.07 |
| H2O transport, Golgi apparatus | | 0.07 |
| proton diffusion (Golgi) | | 0.07 |
| phosphatidate scramblase | | 0.07 |
| phosphatidate scramblase | | 0.07 |
| phosphatidylcholine scramblase | | 0.07 |
| phosphatidylcholine scramblase | | 0.07 |
| choline phosphatase | PLD1; PLD2 | 0.07 |
| choline phosphatase | PLD1 | 0.07 |
| choline phosphatase | PLD1 | 0.07 |
| Phosphatidylserine synthase homo sapiens | PTDSS1 | 0.07 |
| RE3301 | PLD2 | 0.07 |
| Phosphatidylcholine (homo sapiens) exchange | | 0.07 |
| PCHOLHSTDe | | 0.07 |
| H transporter, endoplasmic reticulum | | 0.07 |
| 2-Oxobutanoate dehydrogenase, cytosolic | | 0.07 |
| EC: 1.2.7.2 | BCKDHA; | 0.07 |
| | BCKDHB; DBT; | |
| | DLD | |
| gamma-glutamyltranspeptidase Glutathione | GGT1; GGT5; | 0.07 |
| metabolism EC: 3.4.11.4 | GGT6; GGT7 | |
| Acetylcholine exchange | | 0.07 |
| ADP/ATP transporter, endoplasmic reticulum | | 0.07 |
| RE1441 | PIP4K2A; | 0.07 |
| | PIP4K2B; | |
| | PIP4K2C | |
| RE1448 | TPTE | 0.07 |
| RE1957 | PIP4K2A; | 0.07 |
| | PIP4K2B; | |
| | PIP4K2C | |
| RE3268 | TPTE | 0.07 |
| Neurotransmitter: Sodium Symporter (NSS) | SLC6A6 | 0.07 |
| TCDB: 2.A.22.3.3 | | |
| L-methionine transport via diffusion | SLC43A1; | 0.07 |
| (extracellular to cytosol) | SLC43A2 | |
| taurine transport (sodium symport) (2:1) | SLC6A6 | 0.07 |
| transport of taurine into the intestinal cells by | SLC6A6 | 0.07 |
| beta transport system | | |
| Exchange of hydrosulfide | | 0.07 |
| Free diffusion | | 0.07 |
| Hydroxymethylglutaryl-CoA reversible | | 0.07 |
| mitochondrial transport | | |
| aspartate intake by system ASCT-1 transporter | SLC1A4 | 0.07 |
| glutamate intake by system ASCT-1 transporter | SLC1A4 | 0.07 |
| Postulated transport reaction | | 0.07 |
| 5 alpha dihydrotesterone intracellular transport | | 0.07 |
| 3,4-Dihydroxy-L-phenylalanine exchange | | 0.07 |
| L-leucine transport via diffusion (extracellular to | SLC43A1; | 0.07 |
| cytosol) | SLC43A2 | |
| Histamine uniport | SLC22A2; | 0.07 |
| | SLC22A3; | |
| | SLC22A5 | |
| Resistance-Nodulation-Cell Division (RND) | SLCO1A1 | 0.07 |
| TCDB: 2.A.60.1.14 | | |
| nucleoside-diphosphate kinase (ATP: CDP) | GM20390; | 0.07 |
| | NME2; NME3; | |
| | NME6; NME7 | |
| nucleoside-diphosphate kinase (ATP: GDP) | GM20390; | 0.07 |
| | NME2; NME3; | |
| | NME6; NME7 | |
| Nucleoside-diphosphate kinase (ATP: dGDP) | GM20390; | 0.07 |
| | NME2; NME3; | |
| | NME6; NME7 | |
| nucleoside-diphosphate kinase (ATP: dCDP) | GM20390; | 0.07 |
| | NME2; NME3; | |
| | NME6; NME7 | |
| Na+—K+—Cl— cotransport | SLC12A1; | 0.07 |
| | SLC12A2 | |
| sulfate transport via oxalate countertransport | SLC26A6 | 0.07 |
| (2:1) | | |
| myo-lnositol exchange | | 0.07 |
| myo-lnositol-1-phosphate synthase | 1SYNA1 | 0.07 |
| L-Glutamate 5-semialdehyde: NAD+ | ALDH4A1 | 0.07 |
| oxidoreductase Arginine and proline metabolism | | |
| EC: 1.5.1.12 | | |
| L-glutamate 5-semialdehyde dehydratase, | | 0.07 |
| reversible, mitochondrial | | |
| 1-pyrroline-5-carboxylate dehydrogenase, | ALDH4A1 | 0.07 |
| mitochondrial | | |
| L-4-hydroxyglutamate semialdehyde | ALDH4A1 | 0.07 |
| dehydrogenase, mitochondrial | | |
| L-1-pyrroline-3-hydroxy-5-carboxylate | ALDH4A1 | 0.07 |
| dehydrogenase | | |
| L-1-Pyrroline-3-hydroxy-5-carboxylate | | 0.07 |
| spontaneous conversion to L-4- | | |
| Hydroxyglutamate semialdehyde, mitochondrial | | |
| L-1-Pyrroline-3-hydroxy-5-carboxylate: NADP+ | ALDH4A1 | 0.07 |
| oxidoreductase Arginine and proline metabolism | | |
| EC: 1.5.1.12 | | |
| Glycoside-Pentoside-Hexuronide (GPH): Cation | SLC10A1 | 0.07 |
| Symporter TCDB: 2.A.28.1.1 | | |
| carnitine-propcarnitine carrier, peroxisomal | | 0.07 |
| carnitine O-aceyltransferase, peroxisomal | CRAT | 0.07 |
| transport into the cytosol from peroxisome | | 0.07 |
| (carnitine) | | |
| Postulated transport reaction | | 0.07 |
| Exchange of 1,4-butanediammonium | | 0.07 |
| exchange reaction for pectin-taurocholic acid | | 0.07 |
| complex | | |
| Binding of pectins with taurocholate in the | | 0.07 |
| intestinal lumen, reducing serum cholesterol | | |
| levels. | | |
| coenzyme A transport, peroxisomal | | 0.07 |
| reversible transport of L-Carnitine in | | 0.07 |
| peroxisome | | |
| carnitine dimethyl nonanoyl transferase, | CRAT; CROT | 0.07 |
| revsible, peroxisomal | | |
| carnitine dimethyl nonanoyl transferase, | CRAT; CROT | 0.07 |
| revsible, peroxisomal | | |
| DMNONCOACRNCPT1 | | 0.07 |
| DMNONCOACRNCPT1 | | 0.07 |
| Sterol carrier protein 2 | SCP2 | 0.07 |
| Sterol carrier protein 2 | SCP2 | 0.07 |
| Major Facilitator(MFS) TCDB: 2.A.1.44.1 | SLC43A1 | 0.07 |
| Major Facilitator(MFS) TCDB: 2.A.1.44.1 | SLC43A1 | 0.07 |
| Na+/Ca2+—NH4+ exchange | SLC24A1; | 0.07 |
| | SLC24A2 | |
| Na+/Ca2+—NH4+ exchange | SLC24A1; | 0.07 |
| | SLC24A2 | |
| Sphinganine 1-phosphate exchange | | 0.06 |
| sph1p transport | | 0.06 |
| 4-aminobutyrate reversible transport in via | SLC36A1 | 0.06 |
| proton symport | | |
| 4-aminobutyrate reversible transport in via | SLC6A1; | 0.06 |
| sodium symport (1:2) | SLC6A11; | |
| | SLC6A12; | |
| | SLC6A13 | |
| L-Alanine: 2-oxoglutarate aminotransferase | GPT; GPT2 | 0.06 |
| Glutamate metabolism/Alanine and aspartate | | |
| metabolism EC: 2.6.1.2 | | |
| (R)-3-Amino-2-methylpropanoate: 2-oxoglutarate | ABAT | 0.06 |
| aminotransferase EC: 2.6.1.22 | | |
| D-3-Amino-isobutanoate: pyruvate | AGXT2 | 0.06 |
| aminotransferase, mitochondrial | | |
| (R)-3-Amino-2-methylpropanoate: 2-oxoglutarate | ABAT | 0.06 |
| aminotransferase EC: 2.6.1.22 | | |
| L-Alanine: 2-oxoglutarate aminotransferase | GPT; GPT2 | 0.06 |
| Glutamate metabolism/Alanine and aspartate | | |
| metabolism EC: 2.6.1.2 | | |
| D-3-Amino-isobutanoate: pyruvate | AGXT2 | 0.06 |
| aminotransferase, mitochondrial | | |
| D-fructose transport in via uniport | SLC2A2; | 0.06 |
| | SLC2A5; | |
| | SLC2A7; SLC2A8 | |
| D-fructose transport via sodium cotransport | SLC5A10; | 0.06 |
| | SLC5A9 | |
| phosphate transport in/out via Na+ symporter | SLC20A1; | 0.06 |
| | SLC20A2 | |
| phosphate transport in/out via Na+ symporter | SLC20A1; | 0.06 |
| | SLC20A2 | |
| Resistance-Nodulation-Cell Division (RND) | SLCO1B2 | 0.06 |
| TCDB: 2.A.60.1.5 | | |
| L-alanine transaminase | GPT; GPT2 | 0.06 |
| Transport reaction | | 0.06 |
| RE2799 | | 0.06 |
| exit of D-alanine from the enterocytes | | 0.06 |
| aspartate transaminase | GOT2 | 0.06 |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| transport of L-Ornithine into the cell in exchange | SLC3A1; SLC7A9 | 0.06 |
| for L-Leucine by b0,+AT transporter at the apical | | |
| surfaces of the membranes of small intestine and | | |
| renal cells. | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| transport of L-Ornithine into the cell in exchange | SLC3A1; SLC7A9 | 0.06 |
| for L-Alanine by b0,+AT transporter at the apical | | |
| surfaces of the membranes of small intestine and | | |
| renal cells. | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A.3.8.15 | | |
| Amino Acid-Polyamine-Organocation (APC) | SLC7A9 | 0.06 |
| TCDB: 2.A. 3.8.15 | | |
| Amino Acid-Polyamine-Organocation (APC) | SLC7A9 | 0.06 |
| TCDB: 2.A. 3.8.15 | | |
| Amino Acid-Polyamine-Organocation (APC) | SLC7A9 | 0.06 |
| TCDB: 2.A. 3.8.15 | | |
| Amino Acid-Polyamine-Organocation (APC) | SLC7A9 | 0.06 |
| TCDB: 2.A. 3.8.15 | | |
| Resistance-Nodulation-Cell Division (RND) | SLCO1A1 | 0.06 |
| TCDB: 2.A.60.1.14 | | |
| NADH transporter, peroxisome | | 0.06 |
| carnitine O-aceyltransferase, mitochondrial | CRAT | 0.06 |
| Mitochondria] Carrier (MC) TCDB: 2.A.29.8.3 | SLC25A20 | 0.06 |
| production of octanoylcarnitine | CPT1A; CPT1B; | 0.06 |
| | CPT1C | |
| transport of octanoyl carnitine into the extra | | 0.06 |
| cellular space | | |
| exchange reaction for octanoyl carnitine | | 0.06 |
| exchange reaction for octanoate (n-C8: 0) | | 0.06 |
| fatty-acid--CoA ligase (tetradecanoate) | ACSL3 | 0.06 |
| fatty-acid--CoA ligase (octanoate) | ACSL3 | 0.06 |
| Octanoate transport via diffusion | | 0.06 |
| Ornithine exchange | | 0.06 |
| Diacylglycerol phosphate kinase (homo sapiens) | DGKA; DGKB; | 0.06 |
| | DGKD; DGKE; | |
| | DGKG; DGKH; | |
| | DGKI; DGKQ; | |
| | DGKZ | |
| NADPH: oxidized-thioredoxin oxidoreductase | TXNRD1; | 0.06 |
| Pyrimidine metabolism EC: 1.8.1.9 | TXNRD2 | |
| thioredoxin reductase (NADPH) | TXNRD2; | 0.06 |
| | TXNRD3 | |
| exchange reaction for L-histidine | | 0.06 |
| Histamine exchange | | 0.06 |
| histidine decarboxylase | DDC; HDC | 0.06 |
| K+—Cl— cotransport | SLC12A4; | 0.06 |
| | SLC12A5; | |
| | SLC12A6; | |
| | SLC12A7 | |
| K+—Cl— cotransport | SLC12A4; | 0.06 |
| | SLC12A5; | |
| | SLC12A6; | |
| | SLC12A7 | |
| L-citrulline exchange | | 0.06 |
| Amino Acid-Polyamine-Organocation (APC) | SLC7A2 | 0.06 |
| TCDB: 2.A.3.3.2 | | |
| 2-dehydro-3-deoxy-phosphogluconate aldolase, | | 0.06 |
| mitochondrial | | |
| L-erythro-4-Hydroxyglutamate: 2-oxoglutarate | GOT2 | 0.06 |
| aminotransferase, mitochondrial | | |
| trans-4-Hydroxy-L-proline: NAD+ 5- | PYCR1; PYCR2 | 0.06 |
| oxidoreductase Arginine and proline metabolism | | |
| EC: 1.5.1.2 | | |
| L-1-Pyrroline-3-hydroxy-5-carboxylate: NADP+ | ALDH4A1 | 0.06 |
| oxidoreductase Arginine and proline metabolism | | |
| EC: 1.5.1.12 | | |
| L-erythro-4-Hydroxyglutamate: 2-oxoglutarate | GOT2 | 0.06 |
| aminotransferase 2, mitochondrial | | |
| deoxyribose transport via diffusion | | 0.06 |
| Deoxyribose exchange | | 0.06 |
| Sphingosine 1-phosphate exchange | | 0.06 |
| sphingosine-1-phosphate transport | | 0.06 |
| cholesterol ester (from FULLR2) exchange | | 0.06 |
| D-glucose transport in via proton symport | SLC5A1 | 0.06 |
| ammonia transport via proton antiport | RHAG; RHBG | 0.06 |
| acyl-CoA dehydrogenase (isovaleryl-CoA), | IVD | 0.06 |
| mitochondrial | | |
| 3-Methylbutanoyl-CoA: (acceptor) 2,3- | ACADM; IVD | 0.06 |
| oxidoreductase Valine, leucine and isoleucine | | |
| degradation EC: 1.3.99.10 | | |
| carnitine O-acetyltransferase, reverse direction, | CRAT | 0.06 |
| peroxisomal | | |
| hyaluronan biosynthesis, precursor 1 exchange | | 0.06 |
| tyrosine 3-monooxygenase | TH | 0.06 |
| transport of D-glucose from extracellular space | SLC5A1 | 0.06 |
| to cytosol of mucosal cells in small intestine | | |
| 3-Sulfino-L-alanine carboxy-lyase | CSAD; GAD1; | 0.06 |
| | GAD2 | |
| hypotaurine: NAD+ oxidoreductase Taurine and | | 0.06 |
| hypotaurine metabolism EC: 1.8.1.3 | | |
| AKG transport via sodium symport | SLC13A3 | 0.06 |
| L-2-Aminoadipate-6-semialdehyde: NAD+ 6- | ALDH7A1 | 0.06 |
| oxidoreductase Lysine degradation EC: 1.2.1.31 | | |
| Postulated transport reaction | | 0.06 |
| L-Tryptophan, tetrahydrobiopterin: oxygen | TPH1; TPH2 | 0.06 |
| oxidoreductase (5-hydroxylating) Tryptophan | | |
| metabolism EC: 1.14.16.4 | | |
| RE1903 | | 0.06 |
| acetyl-CoA C-acetyltransferase | ACAT3 | 0.06 |
| Hydroxymethylglutaryl-CoA reversible | | 0.06 |
| peroxisomal transport | | |
| Tyramine O-sulfate exchange | | 0.06 |
| Tyramine O-sulfate transport (diffusion) | | 0.06 |
| Tyramine Sulfotransferase | SULT1A1 | 0.06 |
| glycine reversible transport via proton symport | SLC36A1; | 0.06 |
| | SLC36A2 | |
| Acetate exchange | | 0.06 |
| K+—Cl— cotransporter (NH4+) | SLC12A4; | 0.06 |
| | SLC12A6; | |
| | SLC12A7 | |
| K+—Cl— cotransporter (NH4+) | SLC12A4; | 0.06 |
| | SLC12A6; | |
| | SLC12A7 | |
| RE3326 | BCKDHB | 0.06 |
| sphingoid base-phosphate phosphatase | SGPP1 | 0.06 |
| (sphinganine 1-phosphatase), endoplasmic | | |
| reticulum | | |
| sphingoid base-phosphate phosphatase | SGPP1 | 0.06 |
| (sphinganine 1-phosphatase), endoplasmic | | |
| reticulum | | |
| sph1p intracellular transport | | 0.06 |
| sphinganine intracellular transport | | 0.06 |
| sphingosine intracellular transport | | 0.06 |
| sphingosine kinase 2 | SPHK1; SPHK2 | 0.06 |
| sphingosine-1-phosphate transport | | 0.06 |
| Iodide exchange | | 0.06 |
| L-Thyroxine exchange | | 0.06 |
| Triiodothyronine exchange | | 0.06 |
| lodide: hydrogen-peroxide oxidoreductase 2 | TPO | 0.06 |
| Iodide: hydrogen-peroxide oxidoreductase 3 | TPO | 0.06 |
| Iodide: hydrogen-peroxide oxidoreductase 4 | TPO | 0.06 |
| Iodide: hydrogen-peroxide oxidoreductase | TPO | 0.06 |
| thyroid peroxidase | TPO | 0.06 |
| D-Tagatose exchange | | 0.06 |
| D-Tagatose 1-phosphate D-glyceraldehyde-3- | ALDOB | 0.06 |
| phosphate-lyase | | |
| ketohexokinase (D-tagatose) | KHK | 0.06 |
| D-tagatose uptake via diffusion | | 0.06 |
| exchange reaction for L-cysteine | | 0.05 |
| COA transporter, endoplasmic reticulum | | 0.05 |
| Amino Acid-Polyamine-Organocation (APC) | SLC7A9 | 0.05 |
| TCDB: 2.A.3.8.15 | | |
| Amino Acid-Polyamine-Organocation (APC) | SLC7A9 | 0.05 |
| TCDB: 2.A.3.8.15 | | |
| Amino Acid-Polyamine-Organocation (APC) | SLC7A9 | 0.05 |
| TCDB: 2.A.3.8.15 | | |
| Amino Acid-Polyamine-Organocation (APC) | SLC7A9 | 0.05 |
| TCDB: 2.A.3.8.15 | | |
| Amino Acid-Polyamine-Organocation (APC) | SLC7A9 | 0.05 |
| TCDB: 2.A.3.8.15 | | |
| Amino Acid-Polyamine-Organocation (APC) | SLC7A9 | 0.05 |
| TCDB: 2.A.3.8.15 | | |
| Amino Acid-Polyamine-Organocation (APC) | SLC7A9 | 0.05 |
| TCDB: 2.A.3.8.15 | | |
| Amino Acid-Polyamine-Organocation (APC) | SLC7A9 | 0.05 |
| TCDB: 2.A.3.8.15 | | |
| Amino Acid-Polyamine-Organocation (APC) | SLC7A9 | 0.05 |
| TCDB: 2.A.3.8.15 | | |
| Amino Acid-Polyamine-Organocation (APC) | SLC7A9 | 0.05 |
| TCDB: 2.A.3.8.15 | | |
| Amino Acid-Polyamine-Organocation (APC) | SLC7A9 | 0.05 |
| TCDB: 2.A.3.8.15 | | |
| Amino Acid-Polyamine-Organocation (APC) | SLC7A9 | 0.05 |
| TCDB: 2.A.3.8.15 | | |
| Amino Acid-Polyamine-Organocation (APC) | SLC7A9 | 0.05 |
| TCDB: 2.A.3.8.15 | | |
| nucleotide phosphatase | PNP2 | 0.05 |
| Nicotinate D-ribonucleotide: pyrophosphate | NAPRT; QPRT | 0.05 |
| phosphoribosyltransferase Nicotinate and | | |
| nicotinamide metabolism EC: 2.4.2.11 | | |
| phosphoribosylpyrophosphate synthetase | PRPS1; | 0.05 |
| | PRPS1L1; PRPS2 | |
| Phosphate transporter, peroxisome | | 0.05 |
| Diphosphate transporter, peroxisome | | 0.05 |
| Pyrophosphate phosphohydrolase EC: 3.6.1.1 | LHPP; PPA1; | 0.05 |
| | PPA2 | |
| folate uptake into the enterocytes | SLC46A1 | 0.05 |
| leukotriene E4 exchange | | 0.05 |
| Thiocyanate exchange | | 0.05 |
| L-Lactate exchange | | 0.05 |
| D-lactate dehydrogenase | LDHD | 0.05 |
| Acetylcholinesterase | ACHE | 0.05 |
| sodium/ammonium proton antiporter | SLC9A1; | 0.05 |
| | SLC9A2; SLC9A3 | |
| enolase | ENO1; ENO2; | 0.05 |
| | ENO3 | |
| ascorbic acid oxidase | | 0.05 |
| cytochrome c oxidase, mitochondrial Complex IV | COX411; COX412; | 0.05 |
| | COX5A; COX5B; | |
| | COX6A1; | |
| | COX6A2; | |
| | COX6B1; | |
| | COX6B2; COX6C; | |
| | COX7A1; | |
| | COX7A2; | |
| | COX7A2L; | |
| | COX7B2; COX8A; | |
| | COX8C; MT-CO1; | |
| | MT-CO2; MT- | |
| | CO3 | |
| Superoxide anion exchange | | 0.05 |
| hydrogen peroxide nuclear transport | | 0.05 |
| superoxide anion transport via diffusion | | 0.05 |
| (extracellular) | | |
| superoxide anion transport via diffusion | | 0.05 |
| (mitochondria) | | |
| superoxide anion transport via diffusion | | 0.05 |
| (nucleus) | | |
| superoxide anion transport via diffusion | | 0.05 |
| (peroxisome) | | |
| 02 nuclear transport | | 0.05 |
| superoxide dismutase | SOD1 | 0.05 |
| superoxide dismutase, extracellular | SOD3 | 0.05 |
| superoxide dismutase | SOD2 | 0.05 |
| superoxide dismutase, nuclear | SOD1 | 0.05 |
| superoxide dismutase, peroxisome | SOD1 | 0.05 |
| acyl-CoA dehydrogenase (isobutyryl-CoA), | ACAD8; ACADM | 0.05 |
| mitochondrial | | |
| 3-hydroxyisobutyrate dehydrogenase, | HIBADH | 0.05 |
| mitochondrial | | |
| 2-oxoisovalerate dehydrogenase (acylating; 3- | BCKDHA; | 0.05 |
| methyl-2-oxobutanoate), mitochondrial | BCKDHB; DBT; | |
| | DLD | |
| (S)-3-Hydroxyisobutyryl-CoA hydro-lyase Valine, | ECHS1; | 0.05 |
| leucine and isoleucine degradation EC: 4.2.1.17 | EHHADH; | |
| | HADHA | |
| 3-Hydroxy-2-methylpropanoyl-CoA hydrolase | HIBCH | 0.05 |
| EC: 3.1.2.4 | | |
| H2O endoplasmic reticulum transport | | 0.05 |
| production of hexanoylcarnitine | CPT1A; CPT1B; | 0.05 |
| | CPT1C | |
| transport of hexanoyl carnitine into the extra | | 0.05 |
| cellular fluid | | |
| exchange reaction for hexanoyl carnitine | | 0.05 |
| fatty acid beta oxidation(C8-->C6)m | ACAA2; ACADM; | 0.05 |
| | ECHS1; HADH | |
| transport of Hexanoyl-CoA (n-C6: 0CoA) from | DBI | 0.05 |
| mitochondria into cytosol | | |
| RE3009 | SULT1A1 | 0.05 |
| Facilitated diffusion | | 0.05 |
| thymd transport | SLC29A1 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| Y+LAT2 Utilized transport | SLC7A6 | 0.05 |
| 4 hydroxy retinoic acid demand | | 0.05 |
| 4-hydroxyretinoic acid transport, Nuclear | | 0.05 |
| retinoic acid hydroxylation (P450) | | 0.05 |
| phosphatidylinositol-4,5-bisphosphate 5- | | 0.05 |
| phosphatase, nuclear | | |
| Glycerol: NADP+ oxidoreductase Glycerolipid | AKR1A1; | 0.05 |
| metabolism EC: 1.1.1.2 EC: 1.1.1.72 | AKR1B3 | |
| glycerol kinase | GK; GK2 | 0.05 |
| acetyl-coa transport | SLC33A1 | 0.05 |
| acetyl-coa transport | SLC33A1 | 0.05 |
| COA transporter, endoplasmic reticulum | | 0.05 |
| carnitine O-acetyltransferase, endolasmic | CRAT | 0.05 |
| reticulum | | |
| carnitine O-acetyltransferase, endolasmic | CRAT | 0.05 |
| reticulum | | |
| carnitine O-acetyltransferase | CRAT | 0.05 |
| carnitine O-acetyltransferase | CRAT | 0.05 |
| Mitochondrial Carrier (MC) TCDB: 2.A.29.8.3 | SLC25A20 | 0.05 |
| Mitochondrial Carrier (MC) TCDB: 2.A.29.8.3 | SLC25A20 | 0.05 |
| dehydroepiandrosterone sulfate transport via | SLCO1A1; | 0.05 |
| bicarbonate countertransport | SLCO1B2; | |
| | SLCO2B1 | |
| Organic anion transporter 5 Utilized transport | | 0.05 |
| bilirubin beta-diglucuronide transport via | SLCO1B2 | 0.05 |
| bicarbonate countertransport | | |
| bilirubin monoglucuronide transport via | SLCO1B2 | 0.05 |
| bicarbonate countertransport | | |
| bile acid intracellular transport | | 0.05 |
| leukotriene C4 transport via bicarbonate | SLCO1B2 | 0.05 |
| countertransport | | |
| prostaglandin transport via bicarbonate | SLCO2A1 | 0.05 |
| countertransport | | |
| prostaglandin transport via bicarbonate | SLCO2A1 | 0.05 |
| countertransport | | |
| Prostaglandin H2 transport | | 0.05 |
| Prostaglandin I2 transport | | 0.05 |
| Utilized transport | | 0.05 |
| Organic anion transporter 5 Utilized transport | | 0.05 |
| Organic anion transporter 5 Utilized transport | | 0.05 |
| Organic anion transporter 5 Utilized transport | | 0.05 |
| Organic anion transporter 5 Utilized transport | | 0.05 |
| Organic anion transporter 5 Utilized transport | | 0.05 |
| Organic anion transporter 5 Utilized transport | | 0.05 |
| Organic anion transporter 5 Utilized transport | | 0.05 |
| KHte | | 0.05 |
| pyruvate: [dihydrolipoyllysine-residue | PDHA1; PDHA2; | 0.05 |
| acetyltransferase]-lipoyllysine 2-oxidoreductase | PDHB | |
| (decarboxylating, acceptor-acetylating) | | |
| EC: 1.2.4.1 | | |
| acetyl-CoA: enzyme N6-(dihydrolipoyl)lysine S- | DLAT | 0.05 |
| acetyltransferase Glycolysis/Gluconeogenesis/ | | |
| Alanine and aspartate metabolism/Pyruvate | | |
| metabolism EC: 2.3.1.12 | | |
| phosphatidylserine (homo sapiens) exchange | | 0.05 |
| phosphatidylserine transport | | 0.05 |
| 5-L-Glutamyl-L-alanine exchange | | 0.05 |
| citrate transport via sodium symport | SLC13A2 | 0.05 |
| iron (II) transport | | 0.05 |
| D-proline transport, extracellular | | 0.05 |
| D-proline reversible transport via proton | SLC36A1 | 0.05 |
| symport | | |
| Fe3+ exchange | | 0.05 |
| 3-sulfino-alanine transaminase (irreversible) | GOT1 | 0.05 |
| 3-sulfinopyruvate hydrolase (spotaneous | | 0.05 |
| reaction) | | |
| Sulfate exchange | | 0.05 |
| aspartate transaminase | GOT1 | 0.05 |
| demand reaction for pmtcoa | | 0.05 |
| D-alanine transport via proton symport | SLC36A1 | 0.05 |
| Ethanolamine-phosphate phospho-lyase | ETNPPL | 0.05 |
| (deaminating) EC: 4.2.3.2 | | |
| 2-Oxopropanal: NADP+ oxidoreductase Pyruvate | | 0.05 |
| metabolism EC: 1.2.1.49 | | |
| production of propionylcarnitine | CPT1A; CPT1B; | 0.05 |
| | CPT1C | |
| carnitine O-aceyltransferase, mitochondrial | CRAT | 0.05 |
| transport into the mitochondria from cytosol | SLC25A20 | 0.05 |
| (carnitine) | | |
| transport of butyryl carnitine in the | CPT2 | 0.05 |
| mitochondrial matrix for final hydrolysis | | |
| transport of bytyrylcarnitine into mitochondrial | SLC25A20 | 0.05 |
| matrix | | |
| Beta oxidation of fatty acid | ACADM; ACADS | 0.05 |
| carnitine O-palmitoyltransferase | CPT1A; CPT1B; | 0.05 |
| | CPT1C | |
| carnitine transferase | CPT2 | 0.05 |
| transport into the mitochondria (carnitine) | SLC25A20 | 0.05 |
| fatty acyl-CoA desaturase (n-C18: 1CoA −> n- | FADS2 | 0.05 |
| C18: 2CoA) | | |
| linoelaidic acid exchange | | 0.05 |
| fatty-acid--CoA ligase | ACSL1 | 0.05 |
| fatty acid transport via diffusion | | 0.05 |
| 3,4-Dihydroxy-L-phenylalanine transport | SLC16A10 | 0.05 |
| Active transport | | 0.05 |
| estradiol transport | | 0.05 |
| estradiol exchange | | 0.05 |
| glucose-6-phosphate isomerase | GPI1 | 0.05 |
| Transport of 4-methyl-2-oxopentanoate | | 0.05 |
| Exchange of 4-methyl-2-oxopentanoate | | 0.05 |
| leucine transaminase | BCAT1 | 0.05 |
| nucleoside-diphosphate kinase (ATP: dTDP), | GM20390; NME2 | 0.05 |
| nuclear | | |
| nucleoside-diphosphate kinase (ATP: dTDP), | GM20390; NME2 | 0.05 |
| nuclear | | |
| nucleoside-diphosphate kinase (ATP: dADP), | GM20390; NME2 | 0.05 |
| nuclear | | |
| nucleoside-diphosphate kinase (ATP: dADP), | GM20390; NME2 | 0.05 |
| nuclear | | |
| RE0453 | GM20390; NME2 | 0.05 |
| RE0453 | GM20390; NME2 | 0.05 |
| Acetate exchange | | 0.05 |
| Exchange of 4-ammoniobutanal | | 0.05 |
| spermine monoaldehyde exchange | | 0.05 |
| spermidine monoaldehyde 1 exchange | | 0.05 |
| spermidine monoaldehyde 2 exchange | | 0.05 |
| Exchange of 1,4-butanediammonium | | 0.05 |
| Exchange of spermidine(3+) | | 0.05 |
| Putrescine: oxygen oxidoreductase (deaminating) | AOC1 | 0.05 |
| Urea cycle and metabolism of amino groups | | |
| EC: 1.4.3.6 | | |
| Histidine transport (Na, H coupled) | SLC38A3 | 0.05 |
| triose-phosphate isomerase | TPI1 | 0.05 |
| acyl-CoA oxidase (hexadecanoyl-CoA), | ACOX1 | 0.05 |
| peroxisomal | | |
| hdd2coa intracellular transport | | 0.05 |
| Leukotriene C4 carboxypeptidase | | 0.05 |
| Taurine exchange | | 0.05 |
| UMP exchange | | 0.05 |
| L-lysine transport in via sodium symport | SLC22A5; | 0.05 |
| | SLC6A14 | |
| phosphopantothenoylcysteine decarboxylase | PPCDC | 0.05 |
| ATP: pantothenate 4-phosphotransferase | PANK1; PANK2; | 0.05 |
| Pantothenate and CoA biosynthesis EC: 2.7.1.33 | PANK3; PANK4 | |
| ATP: pantothenate 4-phosphotransferase | PANK1; PANK2; | 0.05 |
| Pantothenate and CoA biosynthesis EC: 2.7.1.33 | PANK3; PANK4 | |
| N-((R)-Pantothenoyl)-L-cysteine carboxy-lyase | | 0.05 |
| Pantothenate and CoA biosynthesis EC: 4.1.1.30 | | |
| N-[(R)-4-Phosphopan tothenoyl]-L-cysteine | PPCDC | 0.05 |
| carboxy-lyase Pantothenate and CoA | | |
| biosynthesis EC: 4.1.1.36 | | |
| ATP: pantothenate 4-phosphotransferase | PANK1; PANK2; | 0.05 |
| Pantothenate and CoA biosynthesis EC: 2.7.1.33 | PANK3; PANK4 | |
| ATP: pantothenate 4-phosphotransferase | PANK1; PANK2; | 0.05 |
| Pantothenate and CoA biosynthesis EC: 2.7.1.33 | PANK3; PANK4 | |
| Facilitated diffusion | | 0.05 |
| Postulated transport reaction | | 0.05 |
| acetyl-CoA C-acetyltransferase, mitochondrial | ACAT1; HADHA; | 0.05 |
| | HADHB | |
| leukotriene D4 exchange | | 0.05 |
| production of 3-O Hdodecanoylcarnitinec | CPT1A; CPT1B; | 0.05 |
| | CPT1C | |
| transport of 3-hydroxydodecanoyl carnitine into | | 0.05 |
| extra cellular space | | |
| exchange reaction for 3-hydroxydodecanoyl | | 0.05 |
| carnitine | | |
| fatty acid beta oxidation(C12-->0HC12)m | ACADM; ECHS1 | 0.05 |
| transport of (S)-3-Hydroxydodecanoyl-CoA from | DBI | 0.05 |
| mitochondria into the cytosol | | |
| nucleoside-diphosphate kinase (ATP: dTDP) | GM20390; | 0.05 |
| | NME2; NME3; | |
| | NME6; NME7 | |
| RE0453 | | 0.05 |
| RE0453 | | 0.05 |
| dIDP nuclear transport | | 0.05 |
| dIDP nuclear transport | | 0.05 |
| dITP nuclear transport | | 0.05 |
| dITP nuclear transport | | 0.05 |
| dTDP nuclear transport | | 0.05 |
| dTDP nuclear transport | | 0.05 |
| dTTP diffusion in nucleus | | 0.05 |
| dTTP diffusion in nucleus | | 0.05 |
| IDP nuclear transport | | 0.05 |
| IDP nuclear transport | | 0.05 |
| ITP nuclear transport | | 0.05 |
| ITP nuclear transport | | 0.05 |
| nucleoside-diphosphate kinase (ATP: dIDP) | GM20390; | 0.05 |
| | NME2; NME3; | |
| | NME6; NME7 | |
| nucleoside-diphosphate kinase (ATP: dIDP) | GM20390; | 0.05 |
| | NME2; NME3; | |
| | NME6; NME7 | |
| nucleoside-diphosphate kinase (ATP: dIDP), | GM20390; NME2 | 0.05 |
| nuclear | | |
| nucleoside-diphosphate kinase (ATP: dIDP), | GM20390; NME2 | 0.05 |
| nuclear | | |
| nucleoside-diphosphate kinase (ATP: IDP), | GM20390; NME2 | 0.05 |
| nuclear | | |
| nucleoside-diphosphate kinase (ATP: IDP), | GM20390; NME2 | 0.05 |
| nuclear | | |
| N-acetylglucosamine 2-epimerase | RENBP | 0.05 |
| N-acetylglucosamine kinase | NAGK | 0.05 |
| phosphoacetylglucosamine mutase | PGM3 | 0.05 |
| UDP-N-acetyl-D-glucosamine 2-epimerase | GNE | 0.05 |
| (Hydrolysis) | | |
| UDP-N-acetylglucosamine diphosphorylase | UAP1; UAP1L1 | 0.05 |
| Bilirubin exchange | | 0.05 |
| diffusion of putriscine into the endothelial cells | | 0.05 |
| O-acetylcarnintine transport into mitochondria | SLC25A20 | 0.05 |
| via diffusion | | |
| Na+/Ca2+—K+ exchange | SLC24A1; | 0.05 |
| | SLC24A2; | |
| | SLC24A3; | |
| | SLC24A4 | |
| Na+/Ca2+—K+ exchange | SLC24A1; | 0.05 |
| | SLC24A2; | |
| | SLC24A3; | |
| | SLC24A4 | |
| exchange reaction for D-Fructose | | 0.05 |
| Beta oxidation of fatty acid | ACADM; ACADS | 0.05 |
| fatty acid intracellular transport | | 0.05 |
| Palmitoyl-CoA: L-carnitine O- | CPT1A; CPT2 | 0.05 |
| palmitoyltransferase Fatty acid metabolism | | |
| EC: 2.3.1.21 | | |
| Palmitoyl-CoA: L-carnitine O- | CPT1A; CPT2 | 0.05 |
| palmitoyltransferase Fatty acid metabolism | | |
| EC: 2.3.1.21 | | |
| Facilitated diffusion | | 0.05 |
| Facilitated diffusion | | 0.05 |
| uptake of food iron by DMT1 transporter | SLC11A2 | 0.04 |
| acetate mitochondrial transport via proton | | 0.04 |
| symport | | |
| Citrate lyase | CLYBL | 0.04 |
| Citrate oxaloacetate-lyase ((pro-3S)-CH2COO— -->acetate) | | 0.04 |
| Citrate cycle (TCA cycle) EC: 4.1.3.6 | | |
| de-Fuc form of PA6 exchange | | 0.04 |
| xylitol: NAD oxidoreductase Pentose and | | 0.04 |
| glucuronate interconversions EC: 1.1.1.15 | | |
| xylulose reductase | DCXR | 0.04 |
| Exchange of 7,8-dihydroneopterin 3- | | 0.04 |
| triphosphate(4-) | | |
| Exchange of Dihydroneopterin | | 0.04 |
| 2-Amino-4-hydroxy-6-(erythro-1,2,3- | ALPL | 0.04 |
| trihydroxypropyl) dihydropteridine triphosphate | | |
| phosphohydrolase (alkaline optimum) Folate | | |
| biosynthesis EC: 3.1.3.1 | | |
| Resistance-Nodulation-Cell Division (RND) | SLCO2A1 | 0.04 |
| TCDB: 2.A.60.1.2 | | |
| transport of suberyl carnitine into the extra | | 0.04 |
| cellular space | | |
| exchange reaction for suberyl carnitine | | 0.04 |
| fatty acid beta oxidation(C10DC-->C8DC) | ACAA1B; ACOX1; | 0.04 |
| | EHHADH; | |
| | HSD17B4 | |
| transport of suberyl carnitine into cytosol | SLC25A20 | 0.04 |
| production of suberyl carnitine | CROT | 0.04 |
| cholesterol precursor intracellular transport | | 0.04 |
| 7-dehydrocholesterol reductase | DHCR7 | 0.04 |
| exchange reaction for cholesterol | | 0.04 |
| Previtamin D3 formation | | 0.04 |
| Vesicular transport | | 0.04 |
| Vitamin D3 formation | | 0.04 |
| dUTP transport via dUDP antiport | SLC25A19 | 0.04 |
| nucleoside-diphosphate kinase (ATP: dUDP), | NME4; NME6 | 0.04 |
| mitochondrial | | |
| ADP/ATP transporter, mitochondrial | SLC25A4; | 0.04 |
| | SLC25A5 | |
| RE2651 | | 0.04 |
| 2-Oxoglutaramate amidohydrolase Glutamate | NIT2 | 0.04 |
| metabolism EC: 3.5.1.3 | | |
| L-Glutamine: pyruvate aminotransferase | | 0.04 |
| Glutamate metabolism EC: 2.6.1.15 | | |
| (S)-Glycerate exchange | | 0.04 |
| L-glycerate export | | 0.04 |
| hydroxypyruvate reductase (NADH) | LDHA; | 0.04 |
| | LDHAL6B; | |
| | LDHB; LDHC; | |
| | LDHD | |
| fatty-acid--CoA ligase | ACSBG2; ACSL1; | 0.04 |
| | ACSL3; ACSL4 | |
| Palmitoyl-CoA hydrolase Fatty acid elongation in | ACOT2; ACOT4; | 0.04 |
| mitochondria EC: 3.1.2.22 EC: 3.1.2.2 | ACOT7; PPT1; | |
| | PPT2 | |
| ATP-binding Cassette (ABC) TCDB: 3.A.1.203.3 | ABCD1 | 0.04 |
| 4-(2-Aminophenyl)-2,4-dioxobutanoate | | 0.04 |
| dehydratase | | |
| RE2349 | KYAT1 | 0.04 |
| Spontaneous reaction | | 0.04 |
| RE1233 | KYAT1 | 0.04 |
| 3-Hydroxy-L-kynurenine: 2-oxoglutarate | AADAT; KYAT1 | 0.04 |
| aminotransferase | | |
| L-Kynurenine: 2-oxoglutarate aminotransferase | AADAT; KYAT1 | 0.04 |
| KHte | | 0.04 |
| Hydroxypyruvate reductase (NADPH) | GRHPR | 0.04 |
| phosphoglycerate dehydrogenase | PHGDH | 0.04 |
| phosphoserine transaminase | PSAT1 | 0.04 |
| phosphoserine phosphatase (L-serine) | PSPH | 0.04 |
| L-Serine: pyruvate aminotransferase Glycine, | AGXT | 0.04 |
| serine and threonine metabolism EC: 2.6.1.51 | | |
| D-Glyceraldehyde: NAD+ oxidoreductase | ALDH1B1; | 0.04 |
| Glycerolipid metabolism EC: 1.2.1.3 | ALDH2; | |
| | ALDH3A2; | |
| | ALDH7A1; | |
| | ALDH9A1 | |
| triokinase | | 0.04 |
| L-alanine transaminase | GPT; GPT2 | 0.04 |
| Exchange of pyridoxal 5-phosphate(2-) | | 0.04 |
| Diffusion | | 0.04 |
| glycerol transport | | 0.04 |
| glycerol kinase | GK; GK2 | 0.04 |
| hypothiocyanite exchange | | 0.04 |
| RE0702 | LPO | 0.04 |
| phosphopentomutase | PGM1; PGM2 | 0.04 |
| Transport of 3-(4-hydroxyphenyl)pyruvate | | 0.04 |
| Exchange of 3-(4-hydroxyphenyl)pyruvate | | 0.04 |
| tyrosine transaminase | GOT1; TAT | 0.04 |
| D-lactate transport via proton symport | SLC16A1; | 0.04 |
| | SLC16A3; | |
| | SLC16A7; | |
| | SLC16A8 | |
| D-lactate exchange | | 0.04 |
| trans-Oct-2-enoyl-CoA reductase Fatty acid | MECR; PECR | 0.04 |
| elongation in mitochondria EC: 1.3.1.38 | | |
| trans-Hex-2-enoyl-CoA reductase Fatty acid | MECR; PECR | 0.04 |
| elongation in mitochondria EC: 1.3.1.38 | | |
| phosphoglucomutase | PGM1; PGM2 | 0.04 |
| DM Asn-X-Ser/Thr(ly) | | 0.04 |
| acylphosphatase | ACYP1; ACYP2; | 0.04 |
| | RWDD2A | |
| Y+LAT2 Utilized transport | SLC7A6 | 0.04 |
| transport of acetylcarnitine from peroxisomes to | SLC25A20 | 0.04 |
| cytosol | | |
| carnitine-acetylcarnitine carrier, peroxisomal | CRAT | 0.04 |
| reversible transport of L-Carnitine in | | 0.04 |
| peroxisome | | |
| Proline transport (sodium symport) (2:1) | SLC6A7 | 0.04 |
| Testosterone exchange | | 0.04 |
| Testosterone transport | | 0.04 |
| transport of dimethylnonanoylcarnitine from | SLC25A20 | 0.04 |
| peroxisomes to cytosol | | |
| transport of dimethylnonanoylcarnitine from | SLC25A20 | 0.04 |
| peroxisomes to cytosol | | |
| carnitine transport peroxisome to mitochondria | | 0.04 |
| carnitine transport peroxisome to mitochondria | | 0.04 |
| 4,8 diniethylnonanoyl carnitine transport | | 0.04 |
| (mitochondria) | | |
| 4,8 diniethylnonanoyl carnitine transport | | 0.04 |
| (mitochondria) | | |
| Transport of Histidine by y+LAT1 or y+LAT2 | SLC3A2; | 0.04 |
| with co-transporter of h in small intestine and | SLC7A6; SLC7A7 | |
| kidney | | |
| ammonia transport via proton antiport | RHAG; RHBG | 0.04 |
| Exchange of N-acetyl-D-mannosamine | | 0.04 |
| UDP-N-acetyl-D-glucosamine 2-epimerase | GNE | 0.04 |
| Aminosugars metabolism EC: 5.1.3.14 | | |
| Drug/Metabolite Transporter (DMT) | SLC35A3 | 0.04 |
| TCDB: 2.A.7.12.7 | | |
| Postulated transport reaction | | 0.04 |
| alanine-glyoxylate transaminase (irreversible), | AGXT2; ETNPPL | 0.04 |
| mitochondrial | | |
| deoxyguanylate kinase (dGMP: dATP) | | 0.04 |
| (mitochondrial) | | |
| enolase | ENO1; ENO2; | 0.04 |
| | ENO3 | |
| phosphoglycerate mutase | BPGM; PGAM1; | 0.04 |
| | PGAM2 | |
| RE2954 | PKM | 0.04 |
| production of 3-hydroxyoctadecenoylcarnitinec | CPT1A; CPT1B; | 0.04 |
| | CPT1C | |
| exchange reaction for 3-hydroxy-octadecenoyl | | 0.04 |
| carnitine | | |
| fatty acid beta oxidation(C18: 1-->C18: 10H)m | ACADVL; | 0.04 |
| | HADHA | |
| transport of 3-hydroxyoctadecenoylcoa from | DB1 | 0.04 |
| mitochondria into the cytosol | | |
| transport of Octadecenoyl-CoA into | SLC25A20 | 0.04 |
| mitochondrial matrix | | |
| transport of Octadecenoyl-CoA into | CPT1A; CPT1B; | 0.04 |
| mitochondrial matrix | CPT1C | |
| transport of Octadecenoyl-CoA into | CPT2 | 0.04 |
| mitochondrial matrix | | |
| transport of 3-hydroxy-octadecenoyl carnitine | | 0.04 |
| into extra cellular space | | |
| OCTDECECOASINK | | 0.04 |
| transport of malonyl carnitine into extra cellular | | 0.04 |
| space | | |
| exchange reaction for malonyl carnitine | | 0.04 |
| malonylcoa->malonylcarnitine | CPT1A; CPT1B; | 0.04 |
| | CPT1C | |
| fatty acid beta oxidation(C6-->C4)m | ACAA2; ACADM; | 0.04 |
| | ECHS1; HADH | |
| fatty acid electroneutral transport | SLC27A1 | 0.04 |
| fatty acid electroneutral transport | SLC27A1 | 0.04 |
| fatty acid transport via diffusion | SLC27A5 | 0.04 |
| fatty acid transport via diffusion | SLC27A5 | 0.04 |
| citrate transport via sodium symport | SLC13A5 | 0.04 |
| Creatine transport (sodium symport) (2:1) | SLC6A8 | 0.04 |
| Na+/iodide cotransport | SLC5A5 | 0.04 |
| Norepinephrine reversible transport in via | SLC6A2; SLC6A3 | 0.04 |
| sodium symport (1:2) | | |
| Norepinephrine uniport | SLC22A1; | 0.04 |
| | SLC22A2; | |
| | SLC22A3 | |
| Neurotransmitter: Sodium Symporter (NSS) | SLC6A8 | 0.04 |
| TCDB: 2.A.22.3.4 | | |
| fatty acid electroneutral transport | SLC27A1; | 0.04 |
| | SLC27A3; | |
| | SLC27A4 | |
| fatty acid transport via diffusion | SLC27A5 | 0.04 |
| fatty acid transport via diffusion | | 0.04 |
| fatty acid transport via diffusion | SLC27A5 | 0.04 |
| fatty acid transport via diffusion | | 0.04 |
| fatty acid transport via diffusion | | 0.04 |
| fatty acid electroneutral transport | SLC27A5 | 0.04 |
| fatty acid electroneutral transport | SLC27A5 | 0.04 |
| fatty acid electroneutral transport | SLC27A2; | 0.04 |
| | SLC27A5 | |
| fatty acid electroneutral transport | SLC27A2; | 0.04 |
| | SLC27A5 | |
| fatty acid electroneutral transport | SLC27A2; | 0.04 |
| | SLC27A5 | |
| fatty acid electroneutral transport | SLC27A2; | 0.04 |
| | SLC27A5 | |
| fatty acid transport via diffusion | SLC27A2 | 0.04 |
| fatty acid transport via diffusion | | 0.04 |
| Active transport | | 0.04 |
| R total exchange | | 0.04 |
| 10-Formyltetrahydrofolate exchange | | 0.04 |
| 7,8-Dihydrofolate exchange | | 0.04 |
| Gamma-glutamyl hydrolase (10FTHF5GLU), | GGH | 0.04 |
| extracellular | | |
| Gamma-glutamyl hydrolase (5DHF), extracellular | GGH | 0.04 |
| Gamma-glutamyl hydrolase (5THF), extracellular | GGH | 0.04 |
| 10-formyltetrahydrofolate-[Glu](5) exchange | | 0.04 |
| 10-formyltetrahydrofolate-[Glu](6) exchange | | 0.04 |
| 10-formyltetrahydrofolate-[Glu](6) exchange | | 0.04 |
| 10-formyltetrahydrofolate-[Glu](7) exchange | | 0.04 |
| pentaglutamyl folate (DHF) exchange | | 0.04 |
| pentaglutamyl folate (THF) exchange | | 0.04 |
| haxglutamyl folate (DHF) exchange | | 0.04 |
| haxglutamyl folate (DHF) exchange | | 0.04 |
| hexaglutamyl folate (THF) exchange | | 0.04 |
| hexaglutamyl folate (THF) exchange | | 0.04 |
| heptaglutamyl folate (DHF) exchange | | 0.04 |
| heptaglutamyl folate (THF) exchange | | 0.04 |
| Gamma-glutamyl hydrolase (10FTHF6GLU), | GGH | 0.04 |
| extracellular | | |
| Gamma-glutamyl hydrolase (10FTHF7GLU), | GGH | 0.04 |
| extracellular | | |
| Gamma-glutamyl hydrolase (6DHF), extracellular | GGH | 0.04 |
| Gamma-glutamyl hydrolase (6THF), extracellular | GGH | 0.04 |
| Gamma-glutamyl hydrolase (7DHF), extracellular | GGH | 0.04 |
| Gamma-glutamyl hydrolase (7THF), extracellular | GGH | 0.04 |
| 10-formyltetrahydrofolate-[Glu](5) exchange | | 0.04 |
| pentaglutamyl folate (DHF) exchange | | 0.04 |
| pentaglutamyl folate (THF) exchange | | 0.04 |
| Cys-Gly exchange | | 0.04 |
| glyoxylate transport, peroxisomal | | 0.04 |
| Amino Acid-Polyamine-Organocation (APC) | SLC7A9 | 0.04 |
| TCDB: 2.A.3.8.15 | | |
| Amino Acid-Polyamine-Organocation (APC) | SLC7A9 | 0.04 |
| TCDB: 2.A.3.8.15 | | |
| Transport of 5-S-methyl-5-thioadenosine | | 0.04 |
| adenosylmethionine decarboxylase | AMD1 | 0.04 |
| DM sprm(c) | | 0.04 |
| Exchange of 5-S-methyl-5-thioadenosine | | 0.04 |
| EX_sprm(e) | | 0.04 |
| spermine synthase | SMS; SRM | 0.04 |
| absorption for spermine across the basolateral | SLC22A1 | 0.04 |
| side | | |
| Amino Acid-Polyamine-Organocation (APC) | SLC7A9 | 0.04 |
| TCDB: 2.A.3.8.15 | | |
| acetate mitochondrial transport via proton | | 0.04 |
| symport | | |
| N-Acetyl-beta-D-glucosaminyl-1,2-alpha-D- | | 0.03 |
| mannosyl-1,3-(N-acetyl-beta-D-glucosaminyl- | | |
| 1,2-alpha-D-mannosyl-l,6)-(N-acetyl-beta-D- | | |
| glucosaminyl-1,4)-beta-D-mannosy]-1,4-N- | | |
| acetyl-beta-D-glucosaminyl-R exchange | | |
| RE3050 | | 0.03 |
| alpha-glucosidase | GAA; GANC; | 0.03 |
| | MGAM | |
| maltose transport (uniport) | SLC2A3 | 0.03 |
| b-ketoacyl synthetase (palmitate, n-C16: 0) | ELOVL2; | 0.03 |
| | ELOVL5; | |
| | ELOVL6; FASN | |
| nucleoside-diphosphate kinase (ATP: UDP) | GM20390; | 0.03 |
| | NME2; NME3; | |
| | NME6; NME7 | |
| exchange reaction for Thiamin | | 0.03 |
| Thiamin monophosphate exchange | | 0.03 |
| Thiamine monophosphate transport in via anion | SLC19A1 | 0.03 |
| antiport | | |
| L-carnitine transport out of mitochondria via | SLC25A20 | 0.03 |
| diffusion | | |
| transport of Octadecenoyl-CoA into | CPT1A; CPT1B; | 0.03 |
| mitochondrial matrix | CPT1C | |
| transport of Octadecenoyl-CoA into | CPT2 | 0.03 |
| mitochondrial matrix | | |
| C181 transport into the mitochondria | SLC25A20 | 0.03 |
| production of butyrylcarnitine | CPT1A; CPT1B; | 0.03 |
| | CPT1C | |
| transport of butyryl carnitine in the | CPT2 | 0.03 |
| mitochondrial matrix for final hydrolysis | | |
| transport of bytyrylcarnitine into mitochondrial | SLC25A20 | 0.03 |
| matrix | | |
| Facilitated diffusion | | 0.03 |
| Postulated transport reaction | | 0.03 |
| sulfate transport via sodium symport | SLC13A4 | 0.03 |
| Mitochondrial Carrier (MC) TCDB: 2.A.29.7.2 | SLC25A1 | 0.03 |
| Mitochondrial Carrier (MC) TCDB: 2.A.29.7.2 | SLC25A1 | 0.03 |
| Mitochondrial Carrier (MC) TCDB: 2.A.29.7.2 | SLC25A1 | 0.03 |
| citrate transport, mitochondrial | SLC25A1 | 0.03 |
| Mitochondrial Carrier (MC) TCDB: 2.A.29.7.2 | SLC25A1 | 0.03 |
| Mitochondrial Carrier (MC) TCDB: 2.A.29.7.2 | SLC25A1 | 0.03 |
| aconitase | ACO1; 1REB2 | 0.03 |
| aconitase | ACO1; 1REB2 | 0.03 |
| Mitochondrial Carrier (MC) TCDB: 2.A.29.7.2 | SLC25A1 | 0.03 |
| Mitochondrial Carrier (MC) TCDB: 2.A.29.7.2 | SLC25A1 | 0.03 |
| chloride transport via formate countertransport | SLC26A4 | 0.03 |
| fatty acid 11-cis-retinol exchange | | 0.03 |
| fatty acid 9-cis-retinol exchange | | 0.03 |
| exchange reaction for lysophosphatidylcholine | | 0.03 |
| Acetyl-CoA: acetyl-CoA C-acetyltransferase Fatty | ACAA1B; ACAA2; | 0.03 |
| acid elongation in mitochondria/Fatty acid | ACAT1; ACAT3; | |
| metabolism EC: 2.3.1.16 | HADHB | |
| (S)-Hydroxyoctanoyl-CoA: NAD+ oxidoreductase | EHHADH; HADH; | 0.03 |
| Fatty acid elongation in mitochondria/Fatty acid | HADHA | |
| metabolism EC: 1.1.1.211 | | |
| (S)-Hydroxyoctanoyl-CoA hydro-lyase Fatty acid | ECHS1; | 0.03 |
| elongation in mitochondria/Fatty acid | EHHADH; | |
| metabolism EC: 4.2.1.17 | HADHA | |
| Hexanoyl-CoA: acetyl-CoA C-acyltransferase Fatty | ACAA1B; ACAA2; | 0.03 |
| acid elongation in mitochondria/Fatty acid | HADHB | |
| metabolism EC: 2.3.1.16 | | |
| (S)-Hydroxyhexanoyl-CoA: NAD+ oxidoreductase | EHHADH; HADH; | 0.03 |
| Fatty acid elongation in mitochondria/Fatty acid | HADHA | |
| metabolism EC: 1.1.1.211 EC: 1.1.1.35 | | |
| (S)-Hydroxyhexanoyl-CoA hydro-lyase Fatty acid | ECHS1; | 0.03 |
| elongation in mitochondria/Fatty acid | EHHADH; | |
| metabolism EC: 4.2.1.17 | HADHA | |
| L-asparaginase (mitochondrial) | ASRGL1 | 0.03 |
| L-asparagine transport, mitochondrial | | 0.03 |
| adenosine facilated transport in cytosol | SLC29A1; | 0.03 |
| | SLC29A2 | |
| Inosine transport (diffusion) | SLC29A1; | 0.03 |
| | SLC29A2 | |
| coenzyme A transport, peroxisomal | | 0.03 |
| phosphoribosylaminoimidazolecarboxamide | ATIC | 0.03 |
| formyltransferase | | |
| Transport of 5-amino-1-(5-phospho-D- | | 0.03 |
| ribosyl)imidazole-4-carboxamide(2-) | | |
| Exchange of 5-amino-1-(5-phospho-D- | | 0.03 |
| ribosyl)imidazole-4-carboxamide(2-) | | |
| IMP cyclohydrolase | ATIC | 0.03 |
| fatty-acid--CoA ligase (hexadecenoate) | ACSBG2; ACSL1 | 0.03 |
| glycine reversible transport via sodium and | SLC6A5 | 0.03 |
| chloride symport (3:1:1) | | |
| glyceraldehyde-3-phosphate dehydrogenase | GAPDHS; | 0.03 |
| | GM15294 | |
| Glutaryl-CoA: (acceptor) 2,3-oxidoreductase | GCDH | 0.03 |
| (decarboxylating) Fatty acid metabolism | | |
| EC: 1.3.99.7 | | |
| fatty acid beta oxidation(C16: 2-->C14: 2)m | ACADVL; | 0.03 |
| | HADHA; HADHB | |
| fatty acid beta oxidation(C18: 2-->C16: 2)m | ACADVL; | 0.03 |
| | HADHA; HADHB | |
| production of decanoylcarnitine | CPT1A; CPT1B; | 0.03 |
| | CPT1C | |
| transport of decanoyl carnitine into extra cellular | | 0.03 |
| space | | |
| exchange reaction for decanoy] carnitine | | 0.03 |
| fatty acyl-CoA synthase (n-C10: 0CoA) | ELOVL2; | 0.03 |
| | ELOVL5; | |
| | ELOVL6; FASN | |
| production of 3-hydroxyhexadecanoylcarnitinec | CPT1A; CPT1B; | 0.03 |
| | CPT1C | |
| exchange reaction for 3-hydroxyhexadecanoyl | | 0.03 |
| carnitine | | |
| fatty acid beta oxidation(C16-->C16OH)m | ACADVL; | 0.03 |
| | HADHA | |
| transport of 3-hydroxyhexadecanoylcoa from | DBI | 0.03 |
| mitochondria into the cytosol | | |
| transport of 3-hydroxyhexadecanoyl carnitine | | 0.03 |
| into extra cellular space | | |
| production of stearoylcarnitine | CPT1A; CPT1B; | 0.03 |
| | CPT1C | |
| carnitine O-stearoyl transferase | CPT2 | 0.03 |
| C180 transport into the mitochondria | SLC25A20 | 0.03 |
| production of 3-hydroxyoctadecanoylcarnitine | CPT1A; CPT1B; | 0.03 |
| | CPT1C | |
| exchange reaction for 3-hydroxyoctadecanoyl | | 0.03 |
| carnitine | | |
| fatty acid beta oxidation(C18-->C180H)m | ACADVL; | 0.03 |
| | HADHA | |
| transport of (S)-3-Hydroxyoctadecanoyl-CoA | DBI | 0.03 |
| from mitochondria into the cytosol | | |
| transport of 3-hydroxyoctadecanoyl carnitine | | 0.03 |
| into extra cellular space | | |
| Exchange of spermidine(3+) | | 0.03 |
| C160 transport into the mitochondria | SLC25A20 | 0.03 |
| Y+LAT2 Utilized transport | SLC7A6 | 0.03 |
| Y+LAT2 Utilized transport | SLC7A6 | 0.03 |
| Y+LAT2 Utilized transport | SLC7A6 | 0.03 |
| Y+LAT2 Utilized transport | SLC7A6 | 0.03 |
| nucleoside-diphosphatase (dCDP) | DCTPP1 | 0.03 |
| methylenetetrahydrofolate dehydrogenase | MTHFD2 | 0.03 |
| (NAD), mitochondrial | | |
| methylenetetrahydrofolate dehydrogenase | MTHFD1; | 0.03 |
| (NADP), mitochondrial | MTHFD1L; | |
| | MTHFD2 | |
| Exchange of 5-O-phosphonato-alpha-D- | | 0.03 |
| ribofuranosyl diphosphate(5-) | | |
| AMP: pyrophosphate phosphoribosyltransferase | APRT; HPRT | 0.03 |
| Purine metabolism EC: 2.4.2.7 | | |
| Dopamine secretion via secretory vesicle (ATP | SLC18A1; | 0.03 |
| driven) | SLC18A2 | |
| Histamine secretion via secretory vesicle (ATP | SLC18A1; | 0.03 |
| driven) | SLC18A2 | |
| palmitoyl-CoA desaturase (n-C16: 0CoA −> n- | SCD4 | 0.03 |
| C16: 1CoA) | | |
| prostaglandine E1 transport (ATP-dependent) | ABCC4 | 0.03 |
| exchange reaction for proton | | 0.03 |
| exchange reaction for pectins | | 0.03 |
| exchange reaction for pectin-deoxycholic acid | | 0.03 |
| complex | | |
| binding of pectins with deoxycholic acid in the | | 0.03 |
| intestinal lumen, reducing serum cholesterol | | |
| levels. | | |
| linoelaidic acid exchange | | 0.03 |
| fatty-acid--CoA ligase | ACSL1 | 0.03 |
| fatty acid transport via diffusion | | 0.03 |
| acetyl-CoA carboxylase | ACACA; ACACB | 0.03 |
| Malonyl-CoA Decarboxylase cytoplasmic | MLYCD | 0.03 |
| Utilized transport | ABCC8; ABCC9; | 0.03 |
| | KCNJ11; KCNJ8 | |
| estradiol intracellular transport | | 0.03 |
| estrone intracellular transport | | 0.03 |
| gulonate dehydrogenase, endoplasmic reticulum | | 0.03 |
| testicular 17-beta-hydroxysteroid | H2-KE6; | 0.03 |
| dehydrogenase | HSD17B1 | |
| testicular 17-beta-hydroxysteroid | HSD17B7 | 0.03 |
| dehydrogenase | | |
| 5′-nucleotidase (dCMP) | GUCA1A; NT5C; | 0.03 |
| | NT5C1A; | |
| | NT5C1B; NT5C3; | |
| | NT5E | |
| ATP: deoxycitidine 5-phosphotransferase | DCK | 0.03 |
| Pyrimidine metabolism EC: 2.7.1.74 | | |
| glycine reversible transport via sodium and | SLC6A5 | 0.03 |
| chloride symport (3:1:1) | | |
| Sedoheptulose 1,7-bisphosphate D- | ALD0ART2; | 0.03 |
| glyceraldehyde-3-phosphate-lyase Carbon | ALDOB; ALDOC | |
| fixation EC: 4.1.2.13 | | |
| transaldolase | TALD01 | 0.03 |
| exchange reaction for ppi | | 0.03 |
| glyceraldehyde-3-phosphate dehydrogenase | GAPDHS; | 0.03 |
| | GM15294 | |
| thiamine-triphosphatase | THTPA | 0.03 |
| thiamine-diphosphate kinase | | 0.03 |
| fatty acid beta oxidation(C4-->C2)m | ACAA2; ACADS; | 0.03 |
| | ECHS1; HADH | |
| 3-hydroxyacyl-CoA dehydratase (3- | AUH; ECHS1; | 0.03 |
| hydroxybutanoyl-CoA) (mitochondria) | HADHA; HADHB | |
| 3-hydroxyacyl-CoA dehydrogenase (acetoacetyl- | HADH; HADHA; | 0.03 |
| CoA) (mitochondria) | HADHB; | |
| | HSD17B10 | |
| S-Adenosyl-L-homocysteine intracellular | | 0.03 |
| diffusion | | |
| S-Adenosyl-L-methionine intracellular diffusion | | 0.03 |
| phosphatidylcholine scramblase | | 0.03 |
| phosphatidylethanolamine N-methyltransferase | PEMT | 0.03 |
| TCDB: 2.A.47.1.9 TCDB: 2.A.29.7.2 | SLC13A5; | 0.03 |
| | SLC25A1 | |
| 3-Hydroxy-L-tyrosine carboxy-lyase | DDC | 0.03 |
| Dopamine exchange | | 0.03 |
| C160 transport into the mitochondria | CPT1A; CPT2 | 0.03 |
| Mitochondrial Carrier (MC) TCDB: 2.A.29.8.3 | SLC25A20 | 0.03 |
| exylitol: NAD oxidoreductase Pentose and | | 0.03 |
| glucuronate interconversions EC: 1.1.1.15 | | |
| xylulose reductase | DCXR | 0.03 |
| Norepinephrine exchange | | 0.03 |
| PTPATe | | 0.03 |
| cysteinesulfinate-aspartate mitochondrial shuttle | SLC25A12; | 0.03 |
| | SLC25A13 | |
| 3-sulfino-alanine transaminase (irreversible), | GOT2 | 0.03 |
| mitochondrial | | |
| 3-sulfinopyruvate hydrolase (spotaneous | | 0.03 |
| reaction), mitochondrial | | |
| EC: 1.1.1.42 | 1DH1; 1DH2; | 0.03 |
| | 1DH3A; 1DH3B; | |
| | 1DH3G | |
| Isocitrate dehydrogenase (NAD+) | 1DH3A; 1DH3B; | 0.03 |
| | 1DH3G | |
| CoA transporter | SLC25A16 | 0.03 |
| EC: 2.3.1.21 | | 0.03 |
| carnitine acetyltransferase EC: 2.3.1.7 | | 0.03 |
| Mitochondrial Carrier (MC) TCDB: 2.A.29.8.3 | SLC25A20 | 0.03 |
| Postulated transport reaction | | 0.03 |
| 17-beta-hydroxysteroid dehydrogenase (type 7) | HSD17B7 | 0.03 |
| 3 beta-hydroxysteroid dehydrogenase type 1 | HSD3B2 | 0.03 |
| 3 beta-hydroxysteroid dehydrogenase type 1 | HSD3B2 | 0.03 |
| Transport of 4-methyl-2-oxopentanoate | | 0.03 |
| Exchange of 4-methyl-2-oxopentanoate | | 0.03 |
| exit of hydroxy-prloine across the basolateral | SLC1A4 | 0.02 |
| surface of enterocytes | | |
| transport of L-OH-Proline by the apical IMINO | SLC6A20B; | 0.02 |
| amino acid transporters in kidney and intestine | TMEM27 | |
| production of palmitoleoylcarnitine | CPT1A; CPT1B; | 0.02 |
| | CPT1C | |
| transport of Hexadecenoyl-CoA into | CPT2 | 0.02 |
| mitochondrial matrix | | |
| C161 transport into the mitochondria | SLC25A20 | 0.02 |
| production of 3-hydroxyhexadecenoylcarnitine | CPT1A; CPT1B; | 0.02 |
| | CPT1C | |
| exchange reaction for 3-hydroxyhexadecenoyl | | 0.02 |
| carnitine | | |
| exchange reaction for Hexadecenoate (n-C16: 1) | | 0.02 |
| fatty-acid--CoA ligase (hexadecenoate) | ACSBG2; ACSL1 | 0.02 |
| production of 3-hydroxyhexadecenoylcoA | ACADVL; | 0.02 |
| | HADHA | |
| transport of 3-hydroxyhexadecenoylcoa from | DB1 | 0.02 |
| mitochondria into the cytosol | | |
| transport of 3-hydroxyhexadecenoyl carnitine | | 0.02 |
| into extra cellular space | | |
| Glycerol: NADP+ oxidoreductase Glycerolipid | AKR1A1; | 0.02 |
| metabolism EC: 1.1.1.2 EC: 1.1.1.72 | AKR1B3 | |
| CO2 peroxisomal transport | | 0.02 |
| Active transport | | 0.02 |
| L-Aspartate exchange | | 0.02 |
| CoA transport in lysosome via diffusion | | 0.02 |
| dephospho-CoA kinase | COASY | 0.02 |
| dephospho-CoA transport from lysosome via | | 0.02 |
| diffusion | | |
| lysosomal acid phosphorylase (CoA) | ACP2; ACP5; | 0.02 |
| | ACP6; ACPP | |
| Facilitated diffusion | | 0.02 |
| carnitine transferase | CPT1A; CPT1B; | 0.02 |
| | CPT1C | |
| carnitine transferase | CPT2 | 0.02 |
| transport into the mitochondria (carnitine) | SLC25A20 | 0.02 |
| Beta oxidation of long chain fatty acid | ACADM; A CADS | 0.02 |
| Beta oxidation of long chain fatty acid | ACADM; ACADS | 0.02 |
| CoA transporter | SLC25A16 | 0.02 |
| Postulated transport reaction | | 0.02 |
| Ca ATPase | ATP2B1; | 0.02 |
| | ATP2B2; | |
| | ATP2B3; | |
| | ATP2B4 | |
| methylenetetrahydrofolate dehydrogenase | MTHFD1; | 0.02 |
| (NADP) | MTHFR | |
| methylenetetrahydrofolate dehydrogenase | MTHFD2; | 0.02 |
| (NAD) | MTHFD2L | |
| Cyanide transport via diffusion (extracellular to | | 0.02 |
| cytosol) | | |
| Hydrogen cyanide exchange | | 0.02 |
| 3-mercaptopyruvate sulfurtransferase | MPST | 0.02 |
| Thiocyanate transport via diffusion (cytosol to | | 0.02 |
| extracellular) | | |
| L-Phenylalanine, tetrahydrobiopterin: oxygen | PAH | 0.02 |
| oxidoreductase (4-hydroxylating) | | |
| L-cysteine transport via diffusion (extracellular | SLC43A2 | 0.02 |
| to cytosol) | | |
| fatty acid beta oxidation(C16-->C14)m | ACADVL; | 0.02 |
| | HADHA; HADHB | |
| transport of Glutamine by y+LAT1 or y+LAT2 | SLC3A2; | 0.02 |
| with co-transporter of h in small intestine and | SLC7A6; SLC7A7 | |
| kidney | | |
| Ornithine exchange | | 0.02 |
| L-ascorbate transport via facilitated diffusion | | 0.02 |
| transport of stearidonylcoa into peroxisomes. | ABCD1 | 0.02 |
| carnitine O-palmitoyltransferase | CPT1A; CPT1B; | 0.02 |
| | OPTIC | |
| C161 transport into the mitochondria | CPT2 | 0.02 |
| C161 transport into the mitochondria | SLC25A20 | 0.02 |
| EC: 6.2.1.3 | ACSBG2; ACSL1; | 0.02 |
| | ACSL3; ACSL4; | |
| | ACSL5; ACSL6; | |
| | SLC27A2 | |
| Acetylcholine exchange | | 0.02 |
| exchange reaction for Choline | | 0.02 |
| Beta oxidation of long chain fatty acid | ACAA1B; ACOX1; | 0.02 |
| | EHHADH; | |
| | HSD17B4 | |
| H transporter, endoplasmic reticulum | | 0.02 |
| NADPH transporter, endoplasmic reticulum | | 0.02 |
| NADP transporter, endoplasmic reticulum | | 0.02 |
| Succinate: ubiquinone oxidoreductase Citrate | SDHA; SDHB; | 0.02 |
| cycle (TCA cycle) EC: 1.3.5.1 | SDHC; SDHD | |
| succinate dehydrogenase | SDHA; SDHB; | 0.02 |
| | SDHC; SDHD | |
| Urea transport via facilitate diffusion | SLC14A1; | 0.02 |
| | SLC14A2; | |
| | SLC5A1; SLC5A5 | |
| urea, water cotransport | SLC5A1 | 0.02 |
| dGTP transport via dADP antiport | SLC25A19 | 0.02 |
| dGTP transport via dUDP antiport | SLC25A19 | 0.02 |
| dGTP transport via dTDP antiport | SLC25A19 | 0.02 |
| dGTP transport via dCDP antiport | SLC25A19 | 0.02 |
| L-phenylalanine transport via diffusion | SLC16A10; | 0.02 |
| (extracellular to cytosol) | SLC43A1; | |
| | SLC43A2 | |
| Seratonin reversible transport in via sodium | SLC6A4 | 0.02 |
| symport/potassium antiport (1:2) | | |
| carnitine O-palmitoyltransferase | CPT1A; CPT1B; | 0.02 |
| | CPT1C | |
| carnitine transferase | CPT2 | 0.02 |
| transport into the mitochondria (carnitine) | SLC25A20 | 0.02 |
| production of 3-hydroxytetradecanoylcarnitine | CPT1A; CPT1B; | 0.02 |
| | CPT1C | |
| exchange reaction for 3-hydroxy-tetradecanoyl | | 0.02 |
| carnitine | | |
| fatty acid beta oxidation(C14-->C14OH)m | ACADVL; | 0.02 |
| | HADHA | |
| transport of (S)-3-Hydroxytetradecanoyl-CoA | DB1 | 0.02 |
| from mitochondria into the cytosol | | |
| transport of 3-hydroxy-tetradecanoyl carnitine | | 0.02 |
| into extra cellular space | | |
| carnitine O-palmitoyltransferase | CPT1A; CPT1B; | 0.02 |
| | CPT1C | |
| carnitine transferase | CPT2 | 0.02 |
| transport into the mitochondria (carnitine) | SLC25A20 | 0.02 |
| production of 3- | CPT1A; CPT1B; | 0.02 |
| hydroxyoctadecadienoylcarnitine | CPT1C | |
| exchange reaction for 3-hydroxyoctadecadienoyl | | 0.02 |
| carnitine | | |
| fatty acid beta oxidation(C18: 2-->C18: 2OH)m | ACADVL; | 0.02 |
| | HADHA | |
| transport of 3-hydroxyoctadecadienoyl carnitine | | 0.02 |
| into extra cellular space | | |
| carnitine O-palmitoyltransferase | CPT1A; CPT1B; | 0.02 |
| | CPT1C | |
| carnitine transferase | CPT2 | 0.02 |
| transport into the mitochondria (carnitine) | SLC25A20 | 0.02 |
| transport of 3-hydroxyoctadecadienoyl coa from | DBI | 0.02 |
| mitochondria into cytosol | | |
| 2-oxoisovalerate dehydrogenase (acylating; 3- | BCKDHA; | 0.02 |
| methyl-2-oxopentanoate), mitochondrial | BCKDHB; DBT; | |
| | DLD | |
| (S)-2-methylbutanoyl-CoA: acceptor 2,3- | ACADM; ACADS; | 0.02 |
| oxidoreductase Valine, leucine and isoleucine | ACADSB | |
| degradation EC: 1.3.99.12 | | |
| phosphatidylcholine flippase | ATP10A | 0.02 |
| phosphatidylethanolamine scramblase | | 0.02 |
| phosphatidylethanolamine N-methyltransferase | PEMT | 0.02 |
| Y+LAT2 Utilized transport | SLC7A6 | 0.02 |
| Y+LAT2 Utilized transport | SLC7A6 | 0.02 |
| Amino Acid-Polyamine-Organocation (APC) | SLC7A9 | 0.02 |
| TCDB: 2.A.3.8.15 | | |
| Y+LAT2 Utilized transport | SLC7A6 | 0.02 |
| Y+LAT2 Utilized transport | SLC7A6 | 0.02 |
| Y+LAT2 Utilized transport | SLC7A6 | 0.02 |
| Y+LAT2 Utilized transport | SLC7A6 | 0.02 |
| Y+LAT2 Utilized transport | SLC7A6 | 0.02 |
| Amino Acid-Polyamine-Organocation (APC) | SLC7A9 | 0.02 |
| TCDB: 2.A.3.8.15 | | |
| Y+LAT2 Utilized transport | SLC7A6 | 0.02 |
| Y+LAT2 Utilized transport | SLC7A6 | 0.02 |
| Y+LAT2 Utilized transport | SLC7A6 | 0.02 |
| Y+LAT2 Utilized transport | SLC7A6 | 0.02 |
| Y+LAT2 Utilized transport | SLC7A6 | 0.02 |
| Y+LAT2 Utilized transport | SLC7A6 | 0.02 |
| Y+LAT2 Utilized transport | SLC7A6 | 0.02 |
| Y+LAT2 Utilized transport | SLC7A6 | 0.02 |
| Arginine/Lysine exchanger (Arg in) | SLC3A2; SLC7A6 | 0.02 |
| 13-cis-oxo-retinoic acid demand | | 0.02 |
| 13-cis-retinoate demand | | 0.02 |
| 4-oxo-retonoic acid demand | | 0.02 |
| 4-oxo-retinoic acid formation (oxidation) | | 0.02 |
| 13-cis-4-oxo-retinoic acid formation (oxidation) | | 0.02 |
| 4-oxo-retinoic acid transport, Nuclear | | 0.02 |
| 13-cis-4-oxo-retinoic acid transport, Nuclear | | 0.02 |
| 13-cis-retinoic acid isomerase | | 0.02 |
| 13-cis-4-oxo-retinoic acid isomerase | | 0.02 |
| 13-cis-4-oxo-retinoic acid isomerase | | 0.02 |
| 13-cis-retinoic acid transnort. nuclear | | 0.02 |
| fatty acid intracellular transport | | 0.02 |
| Beta oxidation of long chain fatty acid | ACAA1B; ACOX1; | 0.02 |
| | EHHADH; | |
| | HSD17B4 | |
| L-lactate dehydrogenase | LDHA; | 0.02 |
| | LDHAL6B; | |
| | LDHB; LDHC; | |
| | UEVLD | |
| dGTP transport via dGDP antiport | SLC25A19 | 0.02 |
| exchange reaction for L-Carnitine | | 0.02 |
| transport of 3,4-Dihydroxy-L-phenylalanine by | SLC3A2; SLC7A5 | 0.02 |
| LAT1 in association with 4F2hc, across the apical | | |
| surface of the membranes. | | |
| 3,4-Dihydroxy-L-phenylalanine transport | SLC16A10 | 0.02 |
| trans-4-Hydroxy-L-proline: NAD+ 5- | PYCR1; PYCR2 | 0.02 |
| oxidoreductase Arginine and proline metabolism | | |
| EC: 1.5.1.2 | | |
| trans-4-Hydroxy-L-proline: NADP+ 5- | PYCR1; PYCR2 | 0.02 |
| oxidoreductase Arginine and proline metabolism | | |
| EC: 1.5.1.2 | | |
| diffusion of S-Adenosyl-L-methionine | | 0.02 |
| diffusion of S-Adenosyl-L-methionine | | 0.02 |
| S-Adenosyl-L-methionine reversible transport, | SLC25A26 | 0.02 |
| mitochondrial | | |
| diffusion of S-Adenosyl-L-homocysteine | | 0.02 |
| diffusion of S-Adenosyl-L-homocysteine | | 0.02 |
| S-Adenosyl-L-methionine reversible transport, | SLC25A26 | 0.02 |
| mitochondrial | | |
| electron transfer flavoprotein | ETFA; ETFB | 0.02 |
| Electron transfer flavoprotein-ubiquinone | ETFDH | 0.02 |
| oxidoreductase | | |
| 1-alkyl 2-acylglycerol 3-phosphocholine | | 0.02 |
| transport | | |
| Platelet-activating factor acetylhydrolase | PLA2G7 | 0.02 |
| 1-alkyl 2-acteylglycerol 3-phosphocholine | | 0.02 |
| transport | | |
| Y+LAT2 Utilized transport | SLC7A6 | 0.02 |
| phosphoglycerate kinase | MIA3; PGK1; | 0.02 |
| | PGK2 | |
| carnitine O-palmitoyltransferase | CPT1A; CPT1B; | 0.02 |
| | CPT1C | |
| carnitine transferase | CPT2 | 0.02 |
| transport into the mitochondria (carnitine) | SLC25A20 | 0.02 |
| Biotin reversible transport via proton symport | SLC16A1 | 0.02 |
| Biotin uptake (antiport) | SLC19A3 | 0.02 |
| deoxyuridine kinase (ATP: Deoxyuridine), | TK2 | 0.02 |
| mitochondrial | | |
| 5′-nucleotidase (dUMP), mitochondrial | NT5M | 0.02 |
| thymidine kinase (ATP: thymidine) | TK2 | 0.02 |
| 5′-nucleotidase (dTMP), mitochondrial | NT5M | 0.02 |
| Mitochondrial Carrier (MC) TCDB: 2.A.29.21.1 | | 0.02 |
| N-Acetyl-beta-D-glucosaminyl-1,2-alpha-D- | | 0.02 |
| mannosyl-1,3-(N-acetyl-beta-D-glucosaminyl- | | |
| 1,2-alpha-D-mannosyl-1,6)-(N-acetyl-beta-D- | | |
| glucosaminyl-1,4)-beta-D-mannosyl-1,4-N- | | |
| acetyl-beta-D-glucosaminyl-R exchange | | |
| trans-Tetradec-2-enoyl-CoA reductase Fatty acid | MECR; PECR | 0.02 |
| elongation in mitochondria EC: 1.3.1.38 | | |
| myristoyl-CoA: acetylCoA C-myristoyltransferase | ACAA1B; ACAA2; | 0.02 |
| Fatty acid elongation in mitochondria/Fatty acid | HADHB | |
| metabolism EC: 2.3.1.16 | | |
| (S)-3-Hydroxyhexadecanoyl-CoA: NAD+ | EHHADH; HADH; | 0.02 |
| oxidoreductase Fatty acid elongation in | HADHA | |
| mitochondria/Fatty acid metabolism | | |
| EC: 1.1.1.211 EC: 1.1.1.35 | | |
| (S)-3-Hydroxyhexadecanoyl-CoA hydro-lyase | ECHS1; | 0.02 |
| Fatty acid elongation in mitochondria/Fatty acid | EHHADH; | |
| metabolism EC: 4.2.1.17 | HADHA | |
| alcohol dehydrogenase (L-1,2-propanediol) | ADH1; ADH4; | 0.02 |
| | ADH5; ADH6A; | |
| | ADH7; ADHFE1; | |
| | ZADH2 | |
| alcohol dehydrogenase (L-lactaldehyde) | ADH1; ADH4; | 0.02 |
| | ADH5; ADH6A; | |
| | ADH7; ADHFE1; | |
| | ZADH2 | |
| aldose reductase (methylglyoxal) | AKR1A1; | 0.02 |
| | AKR1B3; | |
| | AKR7A5 | |
| aldose reductase (acetol) | AKR1A1; | 0.02 |
| | AKR1B3; | |
| | AKR7A5 | |
| acyl-CoA dehydrogenase (butanoyl-CoA), | ACAD11; | 0.02 |
| mitochondrial | ACAD12; | |
| | ACAD8; ACAD9; | |
| | ACADM; ACADS; | |
| | ACADSB | |
| Beta oxidation of long chain fatty acid | ACAA1B; ACOX1; | 0.02 |
| | EHHADH; | |
| | HSD17B4 | |
| fatty acid intracellular transport | | 0.02 |
| fatty acid beta oxidation(C11-->C9)m | ACAA2; ACADM; | 0.02 |
| | ECHS1; HADH | |
| fatty acid beta oxidation(C13-->C11)m | ACADVL; | 0.02 |
| | HADHA; HADHB | |
| fatty acid beta oxidation(C15-->C13)m | ACADVL; | 0.02 |
| | HADHA; HADHB | |
| fatty acid beta oxidation(C5-->C3)m | ACAA2; ACADS; | 0.02 |
| | ECHS1; HADH | |
| fatty acid beta oxidation(C7-->C5)m | ACAA2; ACADM; | 0.02 |
| | ECHS1; HADH | |
| fatty acid beta oxidation(C9-->C7)m | ACAA2; ACADM; | 0.02 |
| | ECHS1; HADH | |
| heptadecanoate exchange | | 0.02 |
| pentadecanoate exchange | | 0.02 |
| fatty-acid--CoA ligase | ACSL1; ACSL3; | 0.02 |
| | ACSL4 | |
| fatty-acid--CoA ligase | ACSL1; ACSL3; | 0.02 |
| | ACSL4 | |
| Beta oxidation of long chain fatty acid (odd | ACADM; ACADS | 0.02 |
| chain) | | |
| Beta oxidation of long chain fatty acid (odd | ACADM | 0.02 |
| chain) | | |
| fatty acid beta oxidation(C17-->C15)m | ACADVL; | 0.02 |
| | HADHA; HADHB | |
| carnitine fatty-acyl transferase | CPT1A; CPT1B; | 0.02 |
| | CPT1C | |
| heptadecanoate transport into the mitochondria | CPT2 | 0.02 |
| heptadecanoate transport into the mitochondria | SLC25A20 | 0.02 |
| fatty acid transport via diffusion | | 0.02 |
| carnitine fatty-acyl transferase | CPT1A; CPT1B; | 0.02 |
| | CPT1C | |
| pentadecanoate transport into the mitochondria | CPT2 | 0.02 |
| pentadecanoate transport into the mitochondria | SLC25A20 | 0.02 |
| fatty acid transport via diffusion | | 0.02 |
| Facilitated diffusion | | 0.02 |
| Complex II reaction for respiratory chain | ETFA; ETFB; | 0.02 |
| | ETFDH | |
| production of myristoylcarnitine | CPT1A; CPT1B; | 0.02 |
| | CPT1C | |
| exchange reaction for myristoyl carnitine | | 0.02 |
| transport of myristoyl carnitine into extra | | 0.02 |
| cellular space | | |
| uptake of uptake of Hexadecanoate by the | | 0.02 |
| enterocytes by the enterocytes | | |
| S-Adenosyl-L-methionine: ethanolamine- | | 0.02 |
| phosphate N-methyltransferase | | |
| Glycerophospholipid metabolism EC: 2.1.1.103 | | |
| S-Adenosyl-L-mcthionine: methylethanolamine | | 0.02 |
| phosphate N-methyltransferase | | |
| Glycerophospholipid metabolism EC: 2.1.1.103 | | |
| S-Adenosyl-L- | | 0.02 |
| methionine: phosphodimethylethanolamine N- | | |
| methyltransferase Glycerophospholipid | | |
| metabolism EC: 2.1.1.103 | | |
| Oxalate exchange | | 0.02 |
| glyoxylate oxidase | LDHA; | 0.02 |
| | LDHAL6B; | |
| | LDHB; LDHC; | |
| | LDHD; UEVLD | |
| L-Arabinitol exchange | | 0.02 |
| L-arabinitol transport via passive diffusion | | 0.02 |
| dCTP transport via dCDP antiport | SLC25A19 | 0.02 |
| dCTP transport via dUDP antiport | SLC25A19 | 0.02 |
| dCTP transport via dGDP antiport | SLC25A19 | 0.02 |
| dCTP transport via dADP antiport | SLC25A19 | 0.02 |
| D-galactose transport via proton symport | SLC5A1 | 0.02 |
| Hydrolase Class (RXN R02973) | VNN1; VNN3 | 0.02 |
| pantothenate kinase | PANK1; PANK2; | 0.02 |
| | PANK3; PANK4 | |
| phosphopantothenate-cysteine ligase | PPCS | 0.02 |
| Cysteamine: oxygen oxidoreductase Taurine and | ADO | 0.02 |
| hypotaurine metabolism EC: 1.13.11.19 | | |
| (R)-4-Phosphopantothenate: L-cysteine ligase | PPCS | 0.02 |
| EC: 6.3.2.5 | | |
| Propionate exchange | | 0.02 |
| guanine deaminase | GDA | 0.02 |
| purine-nucleoside phosphorylase (Guanosine) | PNP2 | 0.02 |
| purine-nucleoside phosphorylase (Xanthosine) | PNP2 | 0.02 |
| Guanosine aminohydrolase EC: 3.5.4.15 | | 0.02 |
| sulfate transport via bicarbonate | SLC26A1; | 0.02 |
| countertransport | SLC26A11; | |
| | SLC26A2; | |
| | SLC26A3; | |
| | SLC26A7; | |
| | SLC26A8; | |
| | SLC26A9 | |
| L-sulfolactate exchange | | 0.01 |
| L-Cysteate: 2-oxoglutarate aminotransferase | GOT1 | 0.01 |
| L-sulfolactate transport (cytosol to extracellular) | | 0.01 |
| L-sulfolactate dehydrogenase (NAD+) | MDH1; MDH1B | 0.01 |
| production of dodecanoylcarnitine | CPT1A; CPT1B; | 0.01 |
| | CPT1C | |
| transport of lauroyl carnitine into extra cellular | | 0.01 |
| space | | |
| exchange reaction for lauroyl carnitine | | 0.01 |
| fatty-acyl-CoA synthase (n-C12: 0CoA) | ELOVL2; | 0.01 |
| | ELOVL5; | |
| | ELOVL6; FASN | |
| 3alpha,7alpha-Dihydroxy-5beta-cholestan-26- | ALDH1B1; | 0.01 |
| al: NAD+ oxidoreductase Bile acid biosynthesis | ALDH2; | |
| EC: 1.2.1.3 | ALDH3A2; | |
| | ALDH7A1; | |
| | ALDH9A1 | |
| RE1807 | | 0.01 |
| L-arginine transport in via sodium symport | SLC6A14 | 0.01 |
| Beta oxidation of long chain fatty acid | ACAA1B; ACOX1; | 0.01 |
| | EHHADH; | |
| | HSD17B4 | |
| nucleoside-diphosphate kinase (ATP: UDP) | GM20390; | 0.01 |
| | NME2; NME3; | |
| | NME6; NME7 | |
| transport of 3-hydroxytetradecenoyl coa from | DBI | 0.01 |
| mitochondria into cytosol | | |
| production of 3-hydroxytetradecenoyl carnitine | CPT1A; CPT1B; | 0.01 |
| | CPT1C | |
| excretion of C14: 1-OH | | 0.01 |
| exchange reaction for 3-hydroxy tetradecenoyl- | | 0.01 |
| 7-carnitine | | |
| fatty acid beta oxidation(C14: 1-->C14: 1OH)m | ACADVL; | 0.01 |
| | HADHA | |
| fatty acid beta oxidation(C16: 1-->C14: 1)m | ACADVL; | 0.01 |
| | HADHA; HADHB | |
| ATP Creatine kinase | CKMT1; CKMT2 | 0.01 |
| ATP Creatine kinase (c) | CKB; CKM | 0.01 |
| Creatine transport to/from mitochondria via | | 0.01 |
| diffusion | | |
| Phosphocreatine transport to/from | | 0.01 |
| mitochondria via diffusion | | |
| activation of docosanoic acid for transport | ACSBG1; | 0.01 |
| | ACSBG2 | |
| transport of behenoylcoa into peroxisomes | ABCD1; ABCD2 | 0.01 |
| fatty acid beta oxidation(C22-->C20)x | ACAA1B; ACOX1; | 0.01 |
| | EHHADH; | |
| | HSD17B4 | |
| phosphatidylcholine transporter | ABCA1; ABCB4 | 0.01 |
| glycogenin self-glucosylation | GYG; GYSI; GYS2 | 0.01 |
| 1,4-alpha-glucan branching enzyme (glygn1 −> | GBE1 | 0.01 |
| glygn2) | | |
| glycogen debranching enzyme | AGL | 0.01 |
| glycogen synthase (ggn −> glygn1) | GYG; GYS1; GYS2 | 0.01 |
| glycogen phosphorylase (glygn2 −> dxtrn) | PYGB; PYGL; | 0.01 |
| | PYGM | |
| glycogen phosphorylase (amyls −> glc-D) | PYGB; PYGL; | 0.01 |
| | PYGM | |
| hydroxy-delta-5-steroid dehydrogenase, 3 beta- | HSD3B7 | 0.01 |
| and steroid delta-isomerase 7 | | |
| RE1796 | HSD3B2 | 0.01 |
| 3 alpha-hydroxysteroid dehydrogenase (type 3) | AKR1C6 | 0.01 |
| 3 alpha-hydroxysteroid dehydrogenase (type 3) | AKR1C6 | 0.01 |
| 3 alpha-hydroxysteroid dehydrogenase (type 3) | AKR1C6 | 0.01 |
| 3 alpha-hydroxysteroid dehydrogenase (type 3) | AKR1C6 | 0.01 |
| leukotriene A4 transport | | 0.01 |
| leukotriene B4 transport | | 0.01 |
| leukotriene D4 transport | | 0.01 |
| leukotriene E4 transport | | 0.01 |
| leukotriene F4 transport | | 0.01 |
| prostaglandin uniport | SLC22A1; | 0.01 |
| | SLC22A2 | |
| Resistance-Nodulation-Cell Division (RND) | SLCO1A1 | 0.01 |
| TCDB: 2.A.60.1.14 | | |
| Resistance-Nodulation-Cell Division (RND) | SLCO1B2 | 0.01 |
| TCDB: 2.A.60.1.5 | | |
| Resistance-Nodulation-Cell Division (RND) | SLCO1B2 | 0.01 |
| TCDB: 2.A.60.1.5 | | |
| Resistance-Nodulation-Cell Division (RND) | SLCO1A1 | 0.01 |
| TCDB: 2.A.60.1.14 | | |
| Resistance-Nodulation-Cell Division (RND) | SLCO1A1 | 0.01 |
| TCDB: 2.A.60.1.14 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Resistance-Nodulation-Cell Division (RND) | SLCO2A1 | 0.01 |
| TCDB: 2.A.60.1.2 | | |
| Major Facilitator(MFS) TCDB: 2.A.1.19.1 | SLC22A1 | 0.01 |
| Organic anion transporter 5 Utilized transport | | 0.01 |
| Organic anion transporter 5 Utilized transport | | 0.01 |
| Organic anion transporter 5 Utilized transport | | 0.01 |
| Organic anion transporter 5 Utilized transport | | 0.01 |
| Organic anion transporter 5 Utilized transport | | 0.01 |
| Organic anion transporter 5 Utilized transport | | 0.01 |
| Organic anion transporter 5 Utilized transport | | 0.01 |
| Organic anion transporter 5 Utilized transport | | 0.01 |
| Organic anion transporter 5 Utilized transport | | 0.01 |
| Organic anion transporter 5 Utilized transport | | 0.01 |
| Organic anion transporter 5 Utilized transport | | 0.01 |
| Organic anion transporter 5 Utilized transport | | 0.01 |
| Organic anion transporter 5 Utilized transport | | 0.01 |
| Organic anion transporter 5 Utilized transport | | 0.01 |
| Organic anion transporter 5 Utilized transport | | 0.01 |
| bile acid intracellular transport | | 0.01 |
| Y+LAT2 Utilized transport | SLC7A6 | 0.01 |
| Y+LAT2 Utilized transport | SLC7A6 | 0.01 |
| Y+LAT2 Utilized transport | SLC7A6 | 0.01 |
| Y+LAT2 Utilized transport | SLC7A6 | 0.01 |
| Y+LAT2 Utilized transport | SLC7A6 | 0.01 |
| 5′-nucleotidase (CMP), extracellular | NT5C; NT5E | 0.01 |
| 3-methyl-2-oxobutanoate mitochondrial | | 0.01 |
| transport via proton symport | | |
| 3-Methyl-2-oxopentanoate mitochondrial | | 0.01 |
| transport via proton symport | | |
| 4-methyl-2-oxopentanoate mitochondrial | | 0.01 |
| transport via proton symport | | |
| Isoleucine mitochondrial transport | | 0.01 |
| isoleucine transaminase | BCAT1 | 0.01 |
| isoleucine transaminase, mitochondrial | BCAT2 | 0.01 |
| leucine mitochondrial transport | | 0.01 |
| leucine transaminase | BCAT1 | 0.01 |
| leucine transaminase, mitochondrial | BCAT2 | 0.01 |
| Valine reversible mitochondrial transport | | 0.01 |
| valine transaminase | BCAT1 | 0.01 |
| valine transaminase, mitochondiral | BCAT2 | 0.01 |
| production of isovalerylcarnitine | CPT1A; CPT1B; | 0.01 |
| | CPT1C | |
| exchange reaction for isovaleryl carnitine | | 0.01 |
| transport of Isovaleryl-CoA from mitochondria | DB1 | 0.01 |
| into cytosol | | |
| transport of isovaleryl carnitine into the extra | | 0.01 |
| cellular space | | |
| Xanthurenic acid exchange | | 0.01 |
| kynurenine 3-monooxygenase | KMO | 0.01 |
| Transport reaction | | 0.01 |
| RE2349 | KYAT1 | 0.01 |
| ABO blood group (transferase A, alpha 1-3-N- | ABO | 0.01 |
| acetylgalactosaminyltransferase; transferase B, | | |
| alpha 1-3-galactosyltransferase) | | |
| ABO blood group (transferase A, alpha 1-3-N- | ABO | 0.01 |
| acetylgalactosaminyltransferase; transferase B, | | |
| alpha 1-3-galactosyltransferase) | | |
| ABO blood group (transferase A, alpha 1-3-N- | ABO | 0.01 |
| acetylgalactosaminyltransferase; transferase B, | | |
| alpha 1-3-galactosyltransferase) | | |
| ABO blood group (transferase A, alpha 1-3-N- | ABO | 0.01 |
| acetylgalactosaminyltransferase; transferase B, | | |
| alpha 1-3-galactosyltransferase) | | |
| ABO blood group (transferase A, alpha 1-3-N- | ABO | 0.01 |
| acetylgalactosaminyltransferase; transferase B, | | |
| alpha 1-3-galactosyltransferase) | | |
| ABO blood group (transferase A, alpha 1-3-N- | ABO | 0.01 |
| acetylgalactosaminyltransferase; transferase B, | | |
| alpha 1-3-galactosyltransferase) | | |
| blood group intracellular transport | | 0.01 |
| blood group intracellular transport | | 0.01 |
| blood group intracellular transport | | 0.01 |
| blood group intracellular transport | | 0.01 |
| UDP-GlcNAc: betaGal beta-1,3-N- | B3GNT2 | 0.01 |
| acetylglucosaminyltransferase 1 | | |
| UDP-GlcNAc: betaGal beta-1,3-N- | B3GNT3 | 0.01 |
| acetylglucosaminyltransferase 3, Golgi apparatus | | |
| UDP-GlcNAc: betaGal beta-1,3-N- | B3GNT3 | 0.01 |
| acetylglucosaminyltransferase 3, Golgi apparatus | | |
| UDP-GlcNAc: betaGal beta-1,3-N- | B3GNT3 | 0.01 |
| acetylglucosaminyltransferase 3, Golgi apparatus | | |
| UDP-GlcNAc: betaGal beta-1,3-N- | B3GNT3 | 0.01 |
| acetylglucosaminyltransferase 3, Golgi apparatus | | |
| Type IIIA glycolipid exchange | | 0.01 |
| Type IIIAb exchange | | 0.01 |
| III3Fuc-nLc6Cer exchange | | 0.01 |
| (Gal)4 (Glc)1 (GlcNAc)2 (LFuc)1 (Cer)1 exchange | | 0.01 |
| (Gal)6 (Glc)1 (GlcNAc)3 (LFuc)2 (Cer)1 exchange | | 0.01 |
| blood group intracellular transport | | 0.01 |
| blood group intracellular transport | | 0.01 |
| Galactoside 2-alpha-L-fucosyltransferase 1 | FUT1 | 0.01 |
| Galactoside 2-alpha-L-fucosyltransferase 1 | FUT1 | 0.01 |
| Galactoside 2-alpha-L-fucosyltransferase 1 | FUT1 | 0.01 |
| Galactoside 2-alpha-L-fucosyltransferase 1 | FUT1 | 0.01 |
| Alpha-(1,3)-fucosyltransferase | FUT9 | 0.01 |
| blood group intracellular transport | | 0.01 |
| blood group intracellular transport | | 0.01 |
| blood group intracellular transport | | 0.01 |
| blood group intracellular transport | | 0.01 |
| N-acetyllactosaminide beta-1,6-N- | GCNT2 | 0.01 |
| acetylglucosaminyl-transferase | | |
| UDP-N-acetylglucosamine 4-epimerase | GALE | 0.01 |
| UDP-GlcNAc: betaGal beta-1,3-N- | B3GNT2 | 0.01 |
| acetylglucosaminyltransferase 1 | | |
| UDP-GlcNAc: betaGal beta-1,3-N- | B3GNT3 | 0.01 |
| acetylglucosaminyltransferase 3, Golgi apparatus | | |
| V3Fuc,III3Fuc-nLc6Cer exchange | | 0.01 |
| (Gal)4 (Glc)1 (GlcNAc)3 (LFuc)3 (Cer)1 exchange | | 0.01 |
| blood group intracellular transport | | 0.01 |
| blood group intracellular transport | | 0.01 |
| blood group intracellular transport | | 0.01 |
| blood group intracellular transport | | 0.01 |
| Alpha-(1,3)-fucosyltransferase | FUT9 | 0.01 |
| Alpha-(1,3)-fucosy)transferase | FUT9 | 0.01 |
| Alpha-(1,3)-fucosyltransferase | FUT9 | 0.01 |
| Alpha-(1,3)-fucosyltransfe rase | FUT9 | 0.01 |
| Alpha-(1,3)-fucosyltransfe rase | FUT9 | 0.01 |
| glucose 6-phosphate dehydrogenase | G6PD2 | 0.01 |
| ATP-binding Cassette (ABC) TCDB: 3.A.1.208.2 | ABCC2 | 0.01 |
| Glycoside-Pentoside-Hexuronide (GPH): Cation | SLC10A1 | 0.01 |
| Symporter TCDB: 2.A.28.1.1 | | |
| sulfate transport via sodium symport | SLC13A4 | 0.01 |
| Seratonin reversible transport in via sodium | SLC6A4 | 0.01 |
| symport/potassium antiport (1:2) | | |
| spermidine synthase | SRM | 0.01 |
| trans-Hexadec-2-enoyl-CoA reductase Fatty acid | MECR; PECR | 0.01 |
| elongation in mitochondria EC: 1.3.1.38 | | |
| 2-Oxoglutarate exchange | | 0.01 |
| (Gal)3 (Glc)1 (GlcNAc)2 (LFuc)3 (Cer)1 exchange | | 0.01 |
| blood group intracellular transport | | 0.01 |
| blood group intracellular transport | | 0.01 |
| Alpha-(1,3)-fucosyltransferase | FL) T9 | 0.01 |
| Alpha-(1,3)-fucosyltransferase | FUT9 | 0.01 |
| GDPFuc Golgi transport via CMP antiport | SLC35C1 | 0.01 |
| GDP intracellular transport | | 0.01 |
| GDP-L-fucose synthase | TSTA3 | 0.01 |
| GDP-D-mannose dehydratase | GMDS | 0.01 |
| GMP transport (Golgi) | | 0.01 |
| GDP-L-fucose: NADP+ 4-oxidoreductase (3,5- | TSTA3 | 0.01 |
| epimerizing) Fructose and mannose metabolism | | |
| EC: 1.1.1.271 | | |
| ABO blood group (transferase A, alpha 1-3-N- | ABO | 0.01 |
| acetylgalactosaminyltransferase; transferase B, | | |
| alpha 1-3-galactosyltransferase) | | |
| ABO blood group (transferase A, alpha 1-3-N- | ABO | 0.01 |
| acetylgalactosaminyltransferase; transferase B, | | |
| alpha 1-3-galactosyltransferase) | | |
| (Gal)2 (GalNAc)1 (Glc)1 (GlcNAc)1 (LFuc)2 | | 0.01 |
| (Cer)1 exchange | | |
| Ley glycolipid exchange | | 0.01 |
| Leb glycolipid exchange | | 0.01 |
| (Gal)3 (Glc)1 (GlcNAc)1 (LFuc)2 (Cer)1 exchange | | 0.01 |
| blood group intracellular transport | | 0.01 |
| blood group intracellular transport | | 0.01 |
| blood group intracellular transport | | 0.01 |
| blood group intracellular transport | | 0.01 |
| blood group intracellular transport | | 0.01 |
| blood group intracellular transport | | 0.01 |
| blood group intracellular transport | | 0.01 |
| blood group intracellular transport | | 0.01 |
| Galactoside 2-alpha-L-fucosyltransferase 1 | FUT1 | 0.01 |
| Galactoside 2-alpha-L-fucosyltransferase 1 | FUT1 | 0.01 |
| fucosyltransferase 3 (galactoside 3(4)-L- | | 0.01 |
| fucosyltransferase, Lewis blood group included) | | |
| 1 | | |
| fucosyltransferase 3 (galactoside 3(4)-L- | | 0.01 |
| fucosyltransferase, Lewis blood group included) | | |
| 1 | | |
| fucosyltransferase 3 (galactoside 3(4)-L- | | 0.01 |
| fucosyltransferase, Lewis blood group included) | | |
| 1 | | |
| Alpha-(1,3)-fucosyltransferase | FUT9 | 0.01 |
| Transport of 3-methyl-2-oxobutanoate | | 0.01 |
| Transport of 3-methyl-2-oxopentanoate | | 0.01 |
| Exchange of 3-methyl-2-oxobutanoate | | 0.01 |
| Exchange of 3-methyl-2-oxopentanoate | | 0.01 |
| L-Isoleucine exchange | | 0.01 |
| L-Valine exchange | | 0.01 |
| isoleucine transaminase | BCAT1 | 0.01 |
| valine transaminase | BCAT1 | 0.01 |
| UDP exchange | | 0.01 |
| nervonic acid exchange | | 0.01 |
| fatty-acid--CoA ligase | ACSL1 | 0.01 |
| Beta oxidation of long chain fatty acid | ACAA1B; ACOX1; | 0.01 |
| | EHHADH; | |
| | HSD17B4 | |
| fatty acid intracellular transport | | 0.01 |
| fatty acid intracellular transport | | 0.01 |
| EC: 1.3.3.6 | ACOX1; ACOX3 | 0.01 |
| beta-N-acetylhexosaminidase Aminosugars | HEXA; HEXB; | 0.01 |
| metabolism EC: 3.2.1.52 | MGEA5 | |
| Transport reaction | | 0.01 |
| dihydrofolate reductase | DHFR | 0.01 |
| 5,6,7,8-Tetrahydrofolate: NAD+ oxidoreductase | DHFR | 0.01 |
| One carbon pool by folate EC: 1.5.1.3 | | |
| D-Fructose 1-phosphate D-glyceraldehyde-3- | ALDOART2; | 0.01 |
| phosphate-lyase | ALDOB; ALDOC | |
| ketohexokinase | KHK | 0.01 |
| Transport of 5-oxoprolinate | | 0.01 |
| Exchange of 5-oxoprolinate | | 0.01 |
| dTMP kinase | DTYMK | 0.01 |
| RE0452 | | 0.01 |
| L-Proline exchange | | 0.01 |
| RE0830 | | 0.01 |
| RE0577 | ACOT2; ACOT6; | 0.01 |
| | BAAT | |
| fatty acyl-CoA desaturase (n-C18: 1CoA −> n- | FADS2 | 0.01 |
| C18: 2CoA) | | |
| Core 5 alpha-GalNAc-transferase, Golgi apparatus | | 0.01 |
| Core 7 alpha-GalNAc-transferase, Golgi apparatus | | 0.01 |
| N-acetylgalactosamine 3-alpha- | | 0.01 |
| galactosyltransferase, Golgi apparatus | | |
| DM core5(g) | | 0.01 |
| DM core7(g) | | 0.01 |
| DM core8(g) | | 0.01 |
| DM sTn antigen(g) | | 0.01 |
| alpha-N-acetylgalactosaminide alpha-2,6- | ST6GALNAC1 | 0.01 |
| sialyltransferase | | |
| L-glutamate secretion via secretory vesicle (ATP | SLC17A6; | 0.01 |
| driven) | SLC17A7; | |
| | SLC17A8 | |
| transport of D-Galactose from extracellular space | SLC5A1 | 0.01 |
| to cytosol of mucosal cells in small intestine | | |
| Octanoyl-CoA: acetyl-CoA C-acyltransferase Fatty | ACAA1B; ACAA2; | 0.01 |
| acid elongation in mitochondria/Fatty acid | HADHB | |
| metabolism EC: 2.3.1.16 | | |
| (S)-Hydroxydecanoyl-CoA: NAD+ oxidoreductase | EHHADH; HADH; | 0.01 |
| Fatty acid elongation in mitochondria/Fatty acid | HADHA | |
| metabolism EC: 1.1.1.35 EC: 1.1.1.211 | | |
| (S)-Hydroxydecanoyl-CoA hydro-lyase Fatty acid | ECHS1; | 0.01 |
| elongation in mitochondria/Fatty acid | EHHADH; | |
| metabolism EC: 4.2.1.17 | HADHA | |
| trans-Dec-2-enoyl-CoA reductase Fatty acid | MECR; PECR | 0.01 |
| elongation in mitochondria EC: 1.3.1.38 | | |
| L-tryptophan transport in via sodium symport | ACE2; SLC6A14; | 0.01 |
| | SLC6A19; | |
| | TMEM27 | |
| L-tyrosine transport in via sodium symport | ACE2; SLC6A14; | 0.01 |
| | SLC6A19; | |
| | TMEM27 | |
| fatty acid beta oxidation(C14-->C12)m | ACADVL; | 0.01 |
| | HADHA; HADHB | |
| Lauroyl-CoA: acetyl-CoA C-acyltransferase Fatty | ACAA1B; ACAA2; | 0.01 |
| acid elongation in mitochondria/Fatty acid | HADHB | |
| metabolism EC: 2.3.1.16 | | |
| (S)-3-Hydroxytetradecanoyl-CoA: NAD+ | EHHADH; HADH; | 0.01 |
| oxidoreductase Fatty acid elongation in | HADHA | |
| mitochondria/Fatty acid metabolism | | |
| EC: 1.1.1.35 EC: 1.1.1.211 | | |
| (S)-3-Hydroxytetradecanoyl-CoA hydro-lyase | ECHS1; | 0.01 |
| Fatty acid elongation in mitochondria/Fatty acid | EHHADH; | |
| metabolism EC: 4.2.1.17 | HADHA | |
| 1-alkyl 2-lysoglycerol 3-phosphocholine | | 0.01 |
| exchange | | |
| 1-alkyl 2-acteylglycerol 3-phosphocholine | | 0.01 |
| (homo sapiens)exchange | | |
| Lecithin-cholesterol acyltransferase | LCAT | 0.01 |
| glycine-cleavage complex (lipoamide), | AMT; DLD; | 0.01 |
| mitochondrial | GCSH; GLDC | |
| glycine-cleavage system (lipoamide) irreversible, | AMT; DLD; | 0.01 |
| mitochondrial | GCSH; GLDC | |
| glycine-cleavage complex (lipoamide), | AMT; DLD; | 0.01 |
| mitochondrial | GCSH; GLDC | |
| glycine-cleavage complex (lipoylprotein), | AMT; DLD; | 0.01 |
| mitochondrial | GCSH; GLDC | |
| glycine-cleavage complex (lipoylprotein) | AMT; DLD; | 0.01 |
| irreversible, mitochondrial | GCSH; GLDC | |
| glycine-cleavage complex (lipoylprotein), | AMT; DLD; | 0.01 |
| mitochondrial | GCSH; GLDC | |
| glycine synthase Nitrogen metabolism | AMT | 0.01 |
| EC: 2.1.2.10 | | |
| L-Glutamate exchange | | 0.01 |
| 2-deoxy-D-ribose 1-phosphate phosphorylase | | 0.01 |
| Deoxyribokinase | RBKS | 0.01 |
| 2-Deoxy-D-ribose 1-phosphate 1,5- | PGM1 | 0.01 |
| phosphomutase Pentose phosphate pathway | | |
| EC: 5.4.2.7 | | |
| transport of taurine into the intestinal cells by | SLC36A1 | 0.01 |
| PAT1 | | |
| Hydroxymethylglutaryl CoA synthase (ir) | HMGCS1 | 0.01 |
| Xanthine: NAD+ oxidoreductase Purine | XDH | 0.01 |
| metabolism EC: 1.17.1.4 | | |
| Hypoxanthine: oxygen oxidoreductase Purine | XDH | 0.01 |
| metabolism EC: 1.17.3.2 | | |
| Gentisate aldehyde: NAD+ oxidoreductase | | 0.01 |
| Tyrosine metabolism EC: 1.2.1.29 | | |
| aldehyde oxidase Tyrosine metabolism EC: 1.2.3.1 | AOX1 | 0.01 |
| (S)-Methylmalonate semialdehyde: NAD+ | ALDH1B1; | 0.01 |
| oxidoreductase Valine, leucine and isoleucine | ALDH2; | |
| degradation EC: 1.2.1.3 | ALDH3A2; | |
| | ALDH7A1; | |
| | ALDH9A1 | |
| (S)-Methylmalonate semialdehyde: oxygen | AOX1 | 0.01 |
| oxidoreductase Valine, leucine and isoleucine | | |
| degradation EC: 1.2.3.1 | | |
| 5-Hydroxyindoleacetaldehyde: oxygen | AOX1 | 0.01 |
| oxidoreductase | | |
| Gentisate aldehyde: NAD+ oxidoreductase | | 0.01 |
| Tyrosine metabolism EC: 1.2.1.29 | | |
| aldehyde oxidase Tyrosine metabolism EC: 1.2.3.1 | AOX1 | 0.01 |
| (S)-Methylmalonate semialdehyde: NAD+ | ALDH1B1; | 0.01 |
| oxidoreductase Valine, leucine and isoleucine | ALDH2; | |
| degradation EC: 1.2.1.3 | ALDH3A2; | |
| | ALDH7A1; | |
| | ALDH9A1 | |
| (S)-Methylmalonate semialdehyde: oxygen | AOX1 | 0.01 |
| oxidoreductase Valine, leucine and isoleucine | | |
| degradation EC: 1.2.3.1 | | |
| 5-Hydroxyindoleacetaldehyde: NAD+ | ALDH1B1; | 0.01 |
| oxidoreductase Tryptophan metabolism | ALDH2; | |
| EC: 1.2.1.3 | ALDH3A2; | |
| | ALDH7A1; | |
| | ALDH9A1 | |
| RE0691 | | 0.01 |
| RE1897 | | 0.01 |
| RE1898 | | 0.01 |
| Spermidine: (acceptor) oxidoreductase | | 0.01 |
| DM Ser/Thr(ly) | | 0.01 |
| exchange reaction for ppi | | 0.01 |
| L-tryptophan transport | SLC16A10; | 0.01 |
| | SLC36A4 | |
| Pyruvate exchange | | 0.01 |
| Hydroxymethylglutaryl-CoA reversible | | 0.01 |
| peroxisomal transport | | |
| RE3272 | | 0.00 |
| TCDB: 2.A.29.2.4 TCDB: 2.A.29.16.1 | SLC25A19; | 0.00 |
| | SLC25A21 | |
| phosphatidylinositol-3,4,5-trisphosphate 3- | PTEN | 0.00 |
| phosphatase, nuclear | | |
| phosphatidylinositol 4,5-bisphosphate 3-kinase, | | 0.00 |
| nuclear | | |
| ATP diphosphohydrolase | ENTPD1; | 0.00 |
| | ENTPD2; | |
| | ENTPD3; | |
| | ENTPD5; | |
| | ENTPD6; | |
| | ENTPD8 | |
| adp exchange | | 0.00 |
| ATP exchange | | 0.00 |
| purine-nucleoside phosphorylase (Inosine) | PNP2 | 0.00 |
| fatty acid beta oxidation(C24: 1-->C22: 1)x | ACAA1B; ACOX1; | 0.00 |
| | EHHADH; | |
| | HSD17B4 | |
| RE3245 | | 0.00 |
| Y+LAT2 Utilized transport | SLC7A6 | 0.00 |
| alpha 1,4-N-acetylglucosaminyltransferase, Golgi | A4GNT | 0.00 |
| apparatus | | |
| DM gncore2(g) | | 0.00 |
| ceramide transport protein | COL4A3BP | 0.00 |
| ceramide transport protein | COL4A3BP | 0.00 |
| Glucosylceramidase | GBA | 0.00 |
| nucleoside-diphosphatase (UDP), endoplasmic | CANT1; | 0.00 |
| reticulum | ENTPD4; | |
| | ENTPD5 | |
| Ceramide glucosyltransferase | UGCG | 0.00 |
| 5′-nucleotidase (AMP), extracellular | NT5C; NT5E | 0.00 |
| transport of L-Carnosine by the apical PEPT1 | SLC15A1 | 0.00 |
| amino acid transporters across the brush border | | |
| cells of the enterocytes of the intestine and renal | | |
| cells | | |
| exchange reaction for carnosine | | 0.00 |
| Beta oxidation of fatty acid | ACADM; ACADS | 0.00 |
| Dolichyl beta-D-glucosyl phosphate flippase | | 0.00 |
| (liver) | | |
| Dolichyl beta-D-glucosyl phosphate flippase | | 0.00 |
| (uterus) | | |
| Dolichyl-beta-D-glucosyl-phosphate | | 0.00 |
| dolichylphosphohydrolase (liver) | | |
| Dolichyl-beta-D-glucosyl-phosphate | | 0.00 |
| dolichylphosphohydrolase (uterus) | | |
| dolichyl-phosphate-mannose-glycolipid alpha- | | 0.00 |
| mannosyltransferase (liver) | | |
| dolichyl-phosphate-mannose-glycolipid alpha- | | 0.00 |
| mannosyltransferase (uterus) | | |
| UDPglucose: dolichyl-phosphate beta-D- | ALG5 | 0.00 |
| glucosyltransferase (liver) | | |
| UDPglucose: dolichyl-phosphate beta-D- | ALG5 | 0.00 |
| glucosyltransferase (uterus) | | |
| lipase | LIPC; LIPF; LIPG; | 0.00 |
| | LPL; PNLIP; | |
| | PNLIPRP1; | |
| | PNLIPRP2 | |
| RTOTAL3 transport | | 0.00 |
| triacylglycerol transport | | 0.00 |
| CO2 endoplasmic reticular transport via diffusion | | 0.00 |
| phosphogluconate dehydrogenase, endoplasmic | PGD | 0.00 |
| reticulum | | |
| 6-phosphogluconolactonase, endoplasmic | H6PD | 0.00 |
| reticulum | | |
| glycine oxidase, perixosomal | | 0.00 |
| RE2659 | | 0.00 |
| Beta oxidation of long chain fatty acid | ACAA1B; ACOX1; | 0.00 |
| | EHHADH; | |
| | HSD17B4 | |
| Beta oxidation of long chain fatty acid | ACAA1B; ACOX1; | 0.00 |
| | EHHADH; | |
| | HSD17B4 | |
| fatty acid intracellular transport | | 0.00 |
| exchange reaction for arachidate | | 0.00 |
| testosterone intracellular transport | | 0.00 |
| Postulated transport reaction | | 0.00 |
| ABC bile acid transporter | ABCB11; ABCC3 | 0.00 |
| phosphatidylethanolamine scramblase | | 0.00 |
| phosphatidylethanolamine flippase | ABCB4; ATP10A; | 0.00 |
| | ATP8A1 | |
| phosphatidylethanolamine flippase | ATP10A; | 0.00 |
| | ATP8A1 | |
| phosphatidylethanolamine transport | | 0.00 |
| phosphatidylserine flippase | ATP10A; | 0.00 |
| | ATP8A1 | |
| phosphatidylserine transport | | 0.00 |
| Transport of 3-(4-hydroxyphenyl)pyruvate | | 0.00 |
| Exchange of 3-i4-hydroxyphenyl)pyruvate | | 0.00 |
| tyrosine transaminase | GOT1; TAT | 0.00 |
| L-Tyrosine exchange | | 0.00 |
| L-tyrosine transport | SLC16A10 | 0.00 |
| sodium/ammonium proton antiporter | SLC9A1; | 0.00 |
| | SLC9A2; SLC9A3 | |
| transport of 3-hydroxy trans 7,10- | DBI | 0.00 |
| hexadecadienoylcoa from mitochondria into | | |
| cytosol | | |
| production of 3-hydroxy hexadecadienoyl | CPT1A; CPT1B; | 0.00 |
| carnitine | CPT1C | |
| excretion of C16: 2OH | | 0.00 |
| exchange reaction for 3-hydroxy trans7,10- | | 0.00 |
| hexadecadienoyl carnitine | | |
| fatty acid beta oxidation(C16: 2-->C16: 2OH)m | ACADVL; | 0.00 |
| | HADHA | |
| fatty acid beta oxidation trans(C18: 2-->C16: 2)m | ACADVL; | 0.00 |
| | HADHA; HADHB | |
| carnitine O-palmitoyltransferase | CPT1A; CPT1B; | 0.00 |
| | CPT1C | |
| carnitine transferase | CPT2 | 0.00 |
| transport into the mitochondria (carnitine) | SLC25A20 | 0.00 |
| Mitochondrial Carrier (MC) TCDB: 2.A.29.7.2 | SLC25A1 | 0.00 |
| Mitochondrial Carrier (MC) TCDB: 2.A.29.7.2 | SLC25A1 | 0.00 |
| nucleoside-diphosphate kinase (ATP: dCDP), | NME4 | 0.00 |
| mitochondrial | | |
| exchange reaction for L-glutamine | | 0.00 |
| dATP diffusion in nucleus | | 0.00 |
| dATP demand | | 0.00 |
| DM datp(n) | | 0.00 |
| RE1447 | | 0.00 |
| inorganic diphosphatase, nuclear | | 0.00 |
| keratan sulfate II (core 2-linked) exchange | | 0.00 |
| keratan sulfate II (core 4-linked) exchange | | 0.00 |
| pyrroline-5-carboxylate reductase (m) | PYCR1 | 0.00 |
| fatty acid intracellular transport | | 0.00 |
| nC22: 6 exchange | | 0.00 |
| fatty-acid--CoA ligase | ACSL1 | 0.00 |
| hexokinase (D-fructose: ATP) | GCK; HK1; HK2; | 0.00 |
| | HK3; HKDC1 | |
| Glycolysis/Glyconeogenesis EC: 2.7.1.1 | HK1; HK2; HK3 | 0.00 |
| Glycolysis/Glyconeogenesis EC: 2.7.1.1 | HK1; HK2; HK3 | 0.00 |
| Leukotriene C4 exchange | | 0.00 |
| prostaglandin Al(1-) exchange | | 0.00 |
| prostaglandin-b1 exchange | | 0.00 |
| prostaglandin C1(1-) exchange | | 0.00 |
| Prostaglandin E1 exchange | | 0.00 |
| RE3556 | | 0.00 |
| RE3565 | | 0.00 |
| RE3568 | | 0.00 |
| timnodonic acid exchange | | 0.00 |
| fatty-acid--CoA ligase | ACSL1 | 0.00 |
| fatty-acid--CoA ligase | ACSL1 | 0.00 |
| fatty acid transport via diffusion | | 0.00 |
| Glycerol phopshate exit into cytosol | | 0.00 |
| citrate transport via sodium symport | SLC13A2 | 0.00 |
| maltopentaose exchange | | 0.00 |
| apelin-13 exchange | | 0.00 |
| Leukotriene D4 dipeptidase | | 0.00 |
| Maltotetraose exchange | | 0.00 |
| Utilized transport | | 0.00 |
| Utilized transport | | 0.00 |
| Vesicular transport | | 0.00 |
| Prostaglandin-H2 D-isomerase [Precursor] | HPGDS; PTGDS | 0.00 |
| Prostaglandin D2 exchange | | 0.00 |
| prostaglandin H2(1-) exchange | | 0.00 |
| prostaglandin I2(1-) exchange | | 0.00 |
| Prostaglandin I2 synthase | PTGIS | 0.00 |
| Prostaglandin I2 transport (ER) | | 0.00 |
| Vesicular transport | | 0.00 |
| solute carrier family 27 (fatty acid transporter), | SLC27A5 | 0.00 |
| member 5 TCDB: 4.C.1.1.5 TCDB: 4.C.1.1.8 | | |
| Utilized transport | | 0.00 |
| Vesicular transport | | 0.00 |
| glycerol-3-phosphate shuttle | | 0.00 |
| Utilized transport | | 0.00 |
| 1-alkyl 2-lysoglycerol 3-phosphocholine | | 0.00 |
| exchange | | |
| 1-alkyl 2-acteylglycerol 3-phosphocholine | | 0.00 |
| (homo sapiens) exchange | | |
| Vesicular transport | | 0.00 |
| Gamma-glutamyltransferase | | 0.00 |
| glycerol transport via channel | | 0.00 |
| RE3524 | | 0.00 |
| urea, water cotransport | SLC5A1 | 0.00 |
| Urea transport via facilitate diffusion | SLC14A1; | 0.00 |
| | SLC14A2; | |
| | SLC5A1; SLC5A5 | |
| acetyl-CoA transport, nuclear | | 0.00 |
| acetyl-CoA transport, nuclear | | 0.00 |
| Acetylcholin transport, nuclear trhough pores | | 0.00 |
| Acetylcholin transport, nuclear trhough pores | | 0.00 |
| ADP transporter, peroxisomal | | 0.00 |
| ADP transporter, peroxisomal | | 0.00 |
| AMP transporter, peroxisome | | 0.00 |
| AMP transporter, peroxisome | | 0.00 |
| intracellular transport | | 0.00 |
| intracellular transport | | 0.00 |
| AMP/ATP transporter, endoplasmic reticulum | | 0.00 |
| ATP transporter, peroxisomal | | 0.00 |
| bile acid Coenzyme A: amino acid N- | BAAT | 0.00 |
| acyltransferase | | |
| bile acid Coenzyme A: amino acid N- | BAAT | 0.00 |
| acyltransferase | | |
| Choline O-acetyltransferase | CHAT | 0.00 |
| Choline O-acetyltransferase | CHAT | 0.00 |
| Choline O-acetyltransferase | CHAT | 0.00 |
| Choline O-acetyltransferase | CHAT | 0.00 |
| Choline transport, nuclear through pores | | 0.00 |
| Choline transport, nuclear through pores | | 0.00 |
| cholesterol intracellular transport | | 0.00 |
| transport of cholesterol into the cytosol | | 0.00 |
| transport of cholesterol into the cytosol | | 0.00 |
| coenzyme A transport, nuclear | | 0.00 |
| bile acid intracellular transport | | 0.00 |
| apelin (1-12) exchange | | 0.00 |
| prostaglandin-a2 exchange | | 0.00 |
| prostaglandin-b1 exchange | | 0.00 |
| prostaglandin-b2 exchange | | 0.00 |
| prostaglandin-c2 exchange | | 0.00 |
| nialtopentaose exchange | | 0.00 |
| Superoxide anion exchange | | 0.00 |
| bile acid intracellular transport | | 0.00 |
| glycine passive transport to peroxisome | | 0.00 |
| palmitate ER export | | 0.00 |
| phosphatidylinositol nuclear transport | | 0.00 |
| (diffusion) | | |
| transport of lysophosphatidylethanolamine into | FABP1 | 0.00 |
| the enterocytes | | |
| phosphatidylethanolamine scramblase | | 0.00 |
| Postulated transport reaction | | 0.00 |
| RE0830 | PCBD1; PCBD2 | 0.00 |
| RE0938 | ACE2 | 0.00 |
| RE1709 | PCBD1; PCBD2 | 0.00 |
| RE2079 | | 0.00 |
| alpha,alpha-trehalase | TREH | 0.00 |
| Fe(II): oxygen oxidoreductase Porphyrin and | CP; FTH1; FTL1; | 0.00 |
| chlorophyll metabolism EC: 1.16.3.1 | FTMT | |
| Choloyl-CoA: glycine N-choloyltransferase Bile | BAAT | 0.00 |
| acid biosynthesis/Taurine and hypotaurine | | |
| metabolism EC: 2.3.1.65 | | |
| Chenodeoxycholate: CoA ligase (AMP-forming) | SLC27A5 | 0.00 |
| Bile acid biosynthesis EC: 6.2.1.7 | | |
| Chenodeoxycholate: CoA ligase (AMP-forming) | SLC27A5 | 0.00 |
| Bile acid biosynthesis EC: 6.2.1.7 | | |
| Chenodeoxycholate: CoA ligase (AMP-forming) | SLC27A5 | 0.00 |
| Bile acid biosynthesis EC: 6.2.1.7 | | |
| glycine N-choloyltransferase EC: 2.3.1.65 | BAAT | 0.00 |
| Mitochondrial Carrier (MC) TCDB: 2.A.29.20.1 | SLC25A17 | 0.00 |
| Mitochondrial Carrier (MC) TCDB: 2.A.29.20.1 | SLC25A17 | 0.00 |
| Utilized transport | | 0.00 |
| Utilized transport | | 0.00 |
| Transport reaction | | 0.00 |
| Facilitated diffusion | | 0.00 |
| Facilitated diffusion | | 0.00 |
| Transport reaction | | 0.00 |
| Transport reaction | | 0.00 |
| Vesicular transport | | 0.00 |
| Resistance-Nodulation-Cell Division (RND) | SLCO2A1 | 0.00 |
| TCDB: 2.A.60.1.2 | | |
| Utilized transport | | 0.00 |
| Utilized transport | | 0.00 |
| Utilized transport | | 0.00 |
| N-acetyl-glucosamine lysosomal efflux | | 0.00 |
| ATP transporter, peroxisomal | | 0.00 |
| prostaglandin Al(1-) exchange | | 0.00 |
| prostaglandin C1(1-) exchange | | 0.00 |
| Trehalose exchange | | 0.00 |
| phosphatidylethanolamine scramblase | | 0.00 |
| RE0830 | PCBD1; PCBD2 | 0.00 |
| RE2660 | PCBD1; PCBD2 | 0.00 |
| diffusion of sorbitol into the enterocytes | | 0.00 |
| transport of succinate by diffusion | | 0.00 |
| succinate transporter, peroxisome | | 0.00 |
| Transport reaction | | 0.00 |
| Free diffusion | | 0.00 |
| Utilized transport | | 0.00 |
| RE3567 | | 0.00 |
| Transport reaction | | 0.00 |
| Transport reaction | | 0.00 |
| solute carrier family 27 (fatty acid transporter), | SLC27A5 | 0.00 |
| member 5 TCDB: 4.C.1.1.5 TCDB: 4.C.1.1.8 | | |
| Vesicular transport | | 0.00 |
| D-sorbitol transport, extracellular | | 0.00 |
| difussion of glycerol accross the brush border | | 0.00 |
| membrane | | |
| Mitochondrial Carrier (MC) TCDB: 2.A.29.7.2 | SLC25A1 | 0.00 |
| Resistance-Nodulation-Cell Division (RND) | SLCO1B2 | 0.00 |
| TCDB: 2.A.60.1.5 | | |
| glycerol-3-phopshate transport, cytoplasm | | 0.00 |
| (5-Glutamyl)-peptide: amino-acid 5- | GGT1; GGT5; | 0.00 |
| glutamyltransferase Arachidonic acid | GGT6; GGT7 | |
| metabolism EC: 2.3.2.2 | | |
| transport of THF into the protal blood | | 0.00 |
| RE1709 | PCBD1; PCBD2 | 0.00 |
| Resistance-Nodulation-Cell Division (RND) | SLCO2A1 | 0.00 |
| TCDB: 2.A.60.1.2 | | |
| transport of ferrous iron into blood | SLC40A1 | 0.00 |
| Gamma-glutamyltransferase | | 0.00 |
| exchange reaction for oxygen | | 0.00 |
| leukotriene F4 exchange | | 0.00 |
| RE3421 | | 0.00 |
| UDP-Glc endoplasmic reticulum transport via | | 0.00 |
| CMP antiport | | |
| 4-Nitrophenol exchange | | 0.00 |
| Exchange of glycerone | | 0.00 |
| Exchange of glycerone phosphate(2-) | | 0.00 |
| Exchange of 4-nitrophenyl phosphate(2-) | | 0.00 |
| Glycerone phosphate phosphohydrolase (alkaline | ALPL | 0.00 |
| optimum) EC: 3.1.3.1 | | |
| 4-Nitrophenyl phosphate phosphohydrolase | ALPL | 0.00 |
| gamma-Hexachlorocyclohexane degradation | | |
| EC: 3.1.3.2 EC: 3.1.3.41 EC: 3.1.3.1 | | |
| Demand for nicotinamide | | 0.00 |
| Nicotinamide exchange | | 0.00 |
| trans-4-Hydroxy-L-proline: NAD+ 5- | PYCR1; PYCR2 | 0.00 |
| oxidoreductase Arginine and proline metabolism | | |
| EC: 1.5.1.2 | | |
| trans-4-Hydroxy-L-proline: NADP+ 5- | PYCR1; PYCR2 | 0.00 |
| oxidoreductase Arginine and proline metabolism | | |
| EC: 1.5.1.2 | | |
| Diphosphoglycerate phosphatase | BPGM; PGAM1; | 0.00 |
| | PGAM2 | |
| Diphosphoglyceromutase | BPGM; PGAM1; | 0.00 |
| | PGAM2 | |
| phosphatidylinositol 4-phosphate nuclear | | 0.00 |
| transport (diffusion) | | |
| L-alanine/L-asparagine Na-dependent exchange | SLC1A5 | 0.00 |
| (Asn-L in) | | |
| L-cysteine/L-asparagine Na-dependent exchange | SLC1A5 | 0.00 |
| (Asn-L in) | | |
| L-glutamine/L-asparagine Na-dependent | SLC1A5 | 0.00 |
| exchange(Asn-L in) | | |
| L-serine/L-asparagine Na-dependent exchange | SLC1A5 | 0.00 |
| (Asn-L in) | | |
| L-threonine/L-asparagine Na-dependent | SLC1A5 | 0.00 |
| exchange(Asn-L in) | | |
| RE3301 | PLD2 | 0.00 |
| glyoxylate transport, mitochondrial | | 0.00 |
| Serotonin uniport | SLC22A1; | 0.00 |
| | SLC22A2 | |
| L-lactate reversible transport via sodium | SLC5A8 | 0.00 |
| symport | | |
| Y+LAT2 Utilized transport | SLC7A6 | 0.00 |
| DM T antigen(g) | | 0.00 |
| inorganic diphosphatase | LHPP; PPA1 | 0.00 |
| Biotin reversible transport via proton symport | SLC16A1 | 0.00 |
| L-aminoadipate-semialdehyde dehydrogenase | | 0.00 |
| (NADH), mitochondrial | | |
| N6-(L-1,3-Dicarboxypropyl)-L-lysine: NAD+ | AASS | 0.00 |
| oxidoreductase; N6-(L-1,3-Dicarboxypropyl)-L- | | |
| lysine: NAD+ oxidoreductase (L-glutamate- | | |
| forming) Lysine degradation EC: 1.5.1.9 | | |
| saccharopine dehydrogenase (NADP, L-lysine | AASS | 0.00 |
| forming), mitochondrial | | |
| Y+LAT2 Utilized transport | SLC7A6 | 0.00 |
| Y+LAT2 Utilized transport | SLC7A6 | 0.00 |
| Y+LAT2 Utilized transport | SLC7A6 | 0.00 |
| RE1903 | | 0.00 |
| L-alanine/L-asparagine Na-dependent exchange | SLC1A5 | 0.00 |
| (Ala-L in) | | |
| L-cysteine/L-asparagine Na-dependent exchange | SLC1A5 | 0.00 |
| (Cys-L in) | | |
| L-serine/L-asparagine Na-dependent exchange | SLC1A5 | 0.00 |
| (Ser-L in) | | |
| L-threonine/L-asparagine Na-dependent | SLC1A5 | 0.00 |
| exchange (Thr-L in) | | |
| L-glutamine/L-asparagine Na-dependent | SLC1A5 | 0.00 |
| exchange (Gln-L in) | | |
| Transport of 3-methyl-2-oxobutanoate | | 0.00 |
| Exchange of 3-methyl-2-oxobutanoate | | 0.00 |
| L-Valine exchange | | 0.00 |
| L-isoleucine transport via diffusion (extracellular | SLC43A1; | 0.00 |
| to cytosol) | SLC43A2 | |
| L-valine transport via diffusion (extracellular to | SLC43A1; | 0.00 |
| cytosol) | SLC43A2 | |
| phosphatidylinositol 3-kinase, endoplasmic | PIK3C2B | 0.00 |
| reticulum | | |
| RE2973 | PIK3C2A; | 0.00 |
| | PIK3C2B; | |
| | PIK3C2G | |
| RE2974 | PIP4K2A; | 0.00 |
| | PIP4K2B; | |
| | PIP4K2C | |
| transport of L-Ascorbate by SVCT1 or SVCT2 | SLC23A1; | 0.00 |
| transporter | SLC23A2 | |
| adenylyl-sulfate kinase | PAPSS1; PAPSS2 | 0.00 |
| 3′,5′-bisphosphate nucleotidase (paps) | BPNT1 | 0.00 |
| activation of adipic acid for formation of adipoyl | | 0.00 |
| carnitine | | |
| transport into cytosol (diffusion) | | 0.00 |
| production of adipoyl carnitine | CROT | 0.00 |
| thioesterification of adipoyl coA for release into | ACOT8 | 0.00 |
| cytosol | | |
| production of sebacoylcarnitine | CPT1A; CPT1B; | 0.00 |
| | CPT1C | |
| transport of sebacoyl carnitine into cytosol | SLC25A20 | 0.00 |
| thioesterification of dodecanedioyl coa for | ACOT8 | 0.00 |
| release into cytosol | | |
| activation of Dodecanedioic acid | | 0.00 |
| transport of Dodecanedioic acid by diffusion | | 0.00 |
| transport of succinyl carnitine into cytosol | SLC25A20 | 0.00 |
| production of adipoylcarnitine | CPT1A; CPT1B; | 0.00 |
| | CPT1C | |
| transport of adipoyl carnitine into cytosol | SLC25A20 | 0.00 |
| production of suberylcarnitine | CPT1A; CPT1B; | 0.00 |
| | CPT1C | |
| formation of dodecanedioyl carnitine | CPT1A; CPT1B; | 0.00 |
| | CPT1C | |
| transport of dodecanedioyl carnitine into cytosol | SLC25A20 | 0.00 |
| production of dodecanedioyl carnitine | CROT | 0.00 |
| succ-->C4DCc | CPT1A; CPT1B; | 0.00 |
| | CPT1C | |
| thioesterification of suberyl coa for release into | ACOT8 | 0.00 |
| cytosol | | |
| activation of Sebacic acid | | 0.00 |
| transport of Sebacic acid into cytosol (diffusion) | | 0.00 |
| production of sebacoyl carnitine | CROT | 0.00 |
| thioesterification of sebacoylcoa for release into | ACOT8 | 0.00 |
| cytosol | | |
| transport of suberic acid into cytosol (diffusion) | | 0.00 |
| transport of suberyl carnitine into cytosol | SLC25A20 | 0.00 |
| production of suberyl carnitine | CROT | 0.00 |
| activation of suberic acid for formation of suberyl | | 0.00 |
| carnitine | | |
| activation of succinate | | 0.00 |
| production of succinyl carnitine | CROT | 0.00 |
| thioesterification of succinyl coa for release into | ACOT4; ACOT8 | 0.00 |
| cytosol | | |
| phosphatidylinositol-3,4,5-trisphosphate 5- | | 0.00 |
| phosphatase, nuclear | | |
| phosphatidylinositol-3,4-bisphosphate 3- | | 0.00 |
| phosphatase, nuclear | | |
| phosphatidylinositol-3,4-bisphosphate 4- | | 0.00 |
| phosphatase, nuclear | | |
| phosphatidylinositol 3,4-bisphosphate 5-kinase, | | 0.00 |
| nuclear | | |
| phosphatidylinositol 3-phosphate 4-kinase, | | 0.00 |
| nuclear | | |
| phosphatidylinositol 4-phosphate 3-kinase, | | 0.00 |
| nuclear | | |
| phosphatidylinositol 4-kinase, nuclear | | 0.00 |
| phosphatidylinositol 3-kinase, nuclear | | 0.00 |
| Y+LAT2 Utilized transport | SLC7A6 | 0.00 |
| Y+LAT2 Utilized transport | SLC7A6 | 0.00 |
| Active transport | | 0.00 |
| fatty acid beta oxidation trans(C10: 1-->C10: 2)m | ACADM | 0.00 |
| fatty acid beta oxidation trans(C10: 2-->C10: 1)m | DECR1 | 0.00 |
| isomerization trans(C12: 2)m | ECI1 | 0.00 |
| fatty acid beta oxidation trans(C12: 2-->C10: 1)m | ACAA2; ECHS1; | 0.00 |
| | HADH | |
| fatty acid beta oxidation trans(C14: 2-->C12: 2)m | ACADVL; | 0.00 |
| | HADHA; HADHB | |
| transport of ubiquinol into cytosol | | 0.00 |
| transport of ubiquinone into mitochondria | | 0.00 |
| galactose transport (uniport) | SLC2A1; | 0.00 |
| | SLC2A10; | |
| | SLC2A2; | |
| | SLC2A3; SLC2A8 | |
| galactose transport via sodium symport | SLC5A10; | 0.00 |
| | SLC5A2; SLC5A9 | |
| mannose transport (uniport) | SLC2A1; | 0.00 |
| | SLC2A2; SLC2A3 | |
| D-mannose transport via sodium cotransport | SLC5A10; | 0.00 |
| | SLC5A9 | |
| Y+LAT2 Utilized transport | SLC7A6 | 0.00 |
| fatty acid intracellular transport | | 0.00 |
| fatty-acyl-CoA elongation (n-C20: 4CoA) | ELOVL2; | 0.00 |
| | ELOVL4; | |
| | ELOVL5; | |
| | ELOVL6 | |
| Beta oxidation of long chain fatty acid | ACAA1B; ACOX1; | 0.00 |
| | EHHADH; | |
| | HSD17B4 | |
| RE0830 | | 0.00 |
| Oxalosuccinate: NADP+ oxidoreductase | IDH1; 1DH2 | 0.00 |
| (decarboxylating) Citrate cycle (TCA cycle) | | |
| EC: 1.1.1.42 | | |
| Isocitrate: NADP+ oxidoreductase | IDH1; IDH2 | 0.00 |
| (decarboxylating) Citrate cycle (TCA cycle) | | |
| EC: 1.1.1.42 | | |
| RE1530 | TK2 | 0.00 |
| production of adipoyl carnitine | CROT | 0.00 |
| transport of adipoyl carnitine into cytosol | SLC25A20 | 0.00 |
| transport of adipoyl carnitine into the extra | | 0.00 |
| cellular fluid | | |
| exchange reaction for adipoyl carnitine | | 0.00 |
| fatty acid beta oxidation(C8DC-->C6DC)x | ACAA1B; ACOX1; | 0.00 |
| | EHHADH; | |
| | HSD17B4 | |
| acetyl-CoA C-acetyltransferase, mitochondrial | ACAA2; ACAT1; | 0.00 |
| | HADHB | |
| 2-Methylprop-2-enoyl-CoA (2-Methylbut-2- | ECHS1; HADHA; | 0.00 |
| enoyl-CoA), mitochondrial | HADHB | |
| 3-hydroxyacyl-CoA dehydrogenase (2- | EHHADH; HADH; | 0.00 |
| Methylacetoacetyl-CoA), mitochondrial | HSD17B10 | |
| citrate hydro-lyase Citrate cycle (TCA cycle)/ | ACO1; ACO2 | 0.00 |
| Glyoxylate and dicarboxylate metabolism | | |
| EC: 4.2.1.4 | | |
| citrate hydro-lyase Citrate cycle (TCA cycle)/ | ACO1; ACO2 | 0.00 |
| Glyoxylate and dicarboxylate metabolism | | |
| EC: 4.2.1.4 | | |
| isocitrate hydro-lyase Citrate cycle (TCA cycle)/ | ACO1; ACO2 | 0.00 |
| Glyoxylate and dicarboxylate metabolism | | |
| EC: 4.2.1.3 | | |
| isocitrate hydro-lyase Citrate cycle (TCA cycle)/ | ACO1; ACO2 | 0.00 |
| Glyoxylate and dicarboxylate metabolism | | |
| EC: 4.2.1.3 | | |
| Aconitate hydratase | ACO1; ACO2 | 0.00 |
| Aconitate hydratase | ACO1; ACO2 | 0.00 |
| lipid, flip-flop intracellular transport | | −0.01 |
| alcohol dehydrogenase Bile acid biosynthesis | ADH1; ADH4; | −0.01 |
| EC: 1.1.1.1 | ADH5; ADH6A; | |
| | ADH7 | |
| Postulated transport reaction | | −0.01 |
| ATP-binding Cassette (ABC) TCDB: 3.A.1.203.1 | ABCD3 | −0.01 |
| hyaluronan biosynthesis, precursor 1 exchange | | −0.01 |
| hyaluronan exchange | | −0.01 |
| hyaluronan synthase | HAS1; HAS2; | −0.01 |
| | HAS3 | |
| hyaluronan synthase | HAS1; HAS2; | −0.01 |
| | HAS3 | |
| lactaldehyde dehydrogenase | ALDH1A1; | −0.01 |
| | ALDH1A2; | |
| | ALDH1A3; | |
| | ALDH3A1; | |
| | ALDH3A2; | |
| | ALDH3B1; | |
| | ALDH3B3; | |
| | ALDH7A1; | |
| | ALDH9A1 | |
| L-lactate dehydrogenase | LDHA; | −0.01 |
| | LDHAL6B; | |
| | LDHB; LDHC; | |
| | UEVLD | |
| octadecenoate (n-C18: 1) exchange | | −0.01 |
| trans-Dodec-2-enoyl-CoA reductase Fatty acid | MECR; PECR | −0.01 |
| elongation in mitochondria EC: 1.3.1.38 | | |
| (S)-3-Hydroxydodecanoyl-CoA hydro-lyase Fatty | ECHS1; | −0.01 |
| acid elongation in mitochondria/Fatty acid | EHHADH; | |
| metabolism EC: 4.2.1.17 | HADHA | |
| (S)-3-Hydroxydodecanoyl-CoA: NAD+ | EHHADH; HADH; | −0.01 |
| oxidoreductase Fatty acid elongation in | HADHA | |
| mitochondria/Fatty acid metabolism | | |
| EC: 1.1.1.35 EC: 1.1.1.211 | | |
| Decanoyl-CoA: acetyl-CoA C-acyltransferase Fatty | ACAA1B; ACAA2; | −0.01 |
| acid elongation in mitochondria/Fatty acid | HADHB | |
| metabolism EC: 2.3.1.16 | | |
| fatty acid transport via diffusion | | −0.01 |
| clupanodonic acid (docosapentaenoic (n-3)) | | −0.01 |
| exchange | | |
| fatty-acid--CoA ligase | ACSL1 | −0.01 |
| L-arabinitol 4-dehydrogenase | | −0.01 |
| succinate dehydrogenase | SDHA; SDHB; | −0.01 |
| | SDHC; SDHD | |
| phosphatase (pan4p) | | −0.01 |
| ATP: pantothenate 4-phosphotransferase | PANK1; PANK2; | −0.01 |
| Pantothenate and CoA biosynthesis EC: 2.7.1.33 | PANK3; PANK4 | |
| Exchange of dGMP(2-) | | −0.01 |
| Exchange of dGMP(2-) | | −0.01 |
| Exchange of dGTP | | −0.01 |
| Exchange of dGTP | | −0.01 |
| 2-Deoxyguanosine 5-triphosphate | ITPA | −0.01 |
| pyrophosphohydrolase Purine metabolism | | |
| EC: 3.6.1.19 | | |
| 2-Deoxyguanosine 5-triphosphate | ITPA | −0.01 |
| pyrophosphohydrolase Purine metabolism | | |
| EC: 3.6.1.19 | | |
| Exchange of Galactosylglycerol | | −0.01 |
| Galactosylglycerol galactohydrolase Galactose | GLA | −0.01 |
| metabolism EC: 3.2.1.22 EC: 3.2.1.23 | | |
| 2-Aminoacrylate sulfotransferase | SULT1A1 | −0.01 |
| Transport of octadecenoyl coA into the | FABP1 | −0.01 |
| enterocytes | | |
| Resistance-Nodulation-Cell Division (RND) | SLCO1A1 | −0.01 |
| TCDB: 2.A.60.1.14 | | |
| xenobiotic transport | | −0.01 |
| 4-Nitrophenol Sulfotransferase | SULT1A1 | −0.01 |
| 4-Nitrophenyl sulfate exchange | | −0.01 |
| fatty-acyl-CoA synthase (n-C14: 0CoA) | ELOVL2; | −0.01 |
| | ELOVL5; | |
| | ELOVL6; FASN | |
| Phosphatidylserine synthase homo sapiens | PTDSS2 | −0.01 |
| Glutamine transport (Na, H coupled) | SLC38A3; | −0.01 |
| | SLC38A5 | |
| alpha-Tocopherol (Vit. E) transport | NPC1L1; | −0.01 |
| | SCARB1 | |
| cholesterol efflux (ATP depedent) | ABCA1; ABCG5; | −0.01 |
| | ABCG8 | |
| fatty acid transport via diffusion | SLC27A5 | −0.01 |
| fatty acid transport via diffusion | SLC27A2 | −0.01 |
| fatty acid transport via diffusion | SLC27A2 | −0.01 |
| Linoleic acid (n-C18: 2) transport in via diffusion | SLC27A5 | −0.01 |
| fatty acid transport via diffusion | SLC27A5 | −0.01 |
| fatty acid transport via diffusion | SLC27A5 | −0.01 |
| ATP-binding Cassette (ABC) TCDB: 3.A.1.211.1 | ABCA1 | −0.01 |
| ATP-binding Cassette (ABC) TCDB: 3.A.1.211.1 | ABCA1 | −0.01 |
| ATP-binding Cassette (ABC) TCDB: 3.A.1.211.1 | ABCA1 | −0.01 |
| ATP-binding Cassette (ABC) TCDB: 3.A.1.211.1 | ABCA1 | −0.01 |
| ATP-binding Cassette (ABC) TCDB: 3.A.1.211.1 | ABCA1 | −0.01 |
| ATP-binding Cassette (ABC) TCDB: 3.A.1.211.1 | ABCA1 | −0.01 |
| ATP-binding Cassette (ABC) TCDB: 3.A.1.211.1 | ABCA1 | −0.01 |
| ATP-binding Cassette (ABC) TCDB: 3.A.1.211.1 | ABCA1 | −0.01 |
| ATP-binding Cassette (ABC) TCDB: 3.A.1.211.1 | ABCA1 | −0.01 |
| ATP-binding Cassette (ABC) TCDB: 3.A.1.211.1 | ABCA1 | −0.01 |
| ATP-binding Cassette (ABC) TCDB: 3.A.1.211.1 | ABCA1 | −0.01 |
| ATP-binding Cassette (ABC) TCDB: 3.A.1.211.1 | ABCA1 | −0.01 |
| ATP-binding Cassette (ABC) TCDB: 3.A.1.211.1 | ABCA1 | −0.01 |
| Proposed Fatty Acid Transporter (FAT) | | −0.01 |
| TCDB: 4.C.1.1.5 | | |
| fatty acid transport via diffusion | | −0.01 |
| tetradecanoate (transport) | SLC27A2 | −0.01 |
| efflux of alpha-Tocopherol into the lymphatics in | ABCA1 | −0.01 |
| chylomicrons | | |
| Acetylcholine secretion via secretory vesicle | SLC18A3 | −0.01 |
| (ATP driven) | | |
| fatty acid intracellular transport | | −0.01 |
| transport of palmitoylcoa into peroxisomes | ABCD1 | −0.01 |
| fatty acid intracellular transport | | −0.01 |
| transport of docosapentenoylcoa into | ABCD1; ABCD2 | −0.01 |
| peroxisomes. | | |
| bile acid intracellular transport | | −0.01 |
| DM atp(c) | | −0.01 |
| ABC bile acid transporter | ABCB11; ABCC3 | −0.01 |
| transport of lignocericyl coenzyme A from | ABCD1; ABCD2 | −0.01 |
| cytosol to peroxisome. | | |
| fatty acid intracellular transport | | −0.01 |
| fatty acid intracellular transport | | −0.01 |
| transport of nervonylcoa into peroxisomes | ABCD1; ABCD2 | −0.01 |
| Phylloquinone transport | | −0.01 |
| transport of pristanoylcoa from cytosol to | ABCD3 | −0.01 |
| peroxisomes. | | |
| pristcoa peroxisomal transport | | −0.01 |
| Amino acid transporter ATB0+ Facilitated | | −0.01 |
| diffusion | | |
| Postulated transport reaction | | −0.01 |
| ATP-binding Cassette (ABC) TCDB: 3.A.1.201.2 | ABCB11 | −0.01 |
| ATP-binding Cassette (ABC) TCDB: 3.A.1.201.2 | ABCB11 | −0.01 |
| Postulated transport reaction | | −0.01 |
| Postulated transport reaction | | −0.01 |
| Postulated transport reaction | | −0.01 |
| ATP-binding Cassette (ABC) TCDB: 3.A.1.211.1 | | −0.01 |
| Proposed Fatty Acid Transporter (FAT) | | −0.01 |
| TCDB: 4.C.1.1.5 | | |
| ATP-binding Cassette (ABC) TCDB: 3.A.1.203.3 | ABCD1 | −0.01 |
| ATP-binding Cassette (ABC) TCDB: 3.A.1.203.3 | ABCD1 | −0.01 |
| ATP-binding Cassette (ABC) TCDB: 3.A.1.203.3 | ABCD1 | −0.01 |
| riboflavin transport (ATP dependent! | SLC52A3 | −0.01 |
| release of riboflavin into the portal blood. | SLC52A2 | −0.01 |
| fatty acid intracellular transport | | −0.01 |
| transport of Stearoyl-CoA from cytosol to | ABCD1 | −0.01 |
| peroxisomes | | |
| transport of vit K into lymph | | −0.01 |
| transport of phytanoylcoa from cytosol to | ABCD3 | −0.01 |
| peroxisomes. | | |
| Sterol carrier protein 2 | SCP2 | −0.01 |
| adenosine kinase | | −0.01 |
| adenosine kinase | | −0.01 |
| deoxyadenosine kinase | | −0.01 |
| NICRNS | | −0.01 |
| Nicotinate D-ribonucleotide phosphohydrolase | NT5C; NT5C1A; | −0.01 |
| | NT5C1B; NT5C2; | |
| | NT5C3; NT5E | |
| 5′-nucleotidase (dAMP) | NT5C; NT5C1A; | −0.01 |
| | NT5C1B; NT5C2; | |
| | NT5C3; NT5E | |
| RE1530 | | −0.01 |
| Beta oxidation of long chain fatty acid | ACAA1B; ACOX1; | −0.01 |
| | EHHADH; | |
| | HSD17B4 | |
| RE2985 | ACOX1; ACOX2; | −0.01 |
| | ACOX3 | |
| RE2986 | EHHADH | −0.01 |
| RE2987 | EHHADH; | −0.01 |
| | HSD17B4 | |
| RE2988 | ACAA1B | −0.01 |
| RE2996 | DECR2 | −0.01 |
| RE3002 | ECI3; EHHADH | −0.01 |
| binding of betaglucans with glycocholate in the | | −0.01 |
| intestinal lumen, reducing serum cholesterol | | |
| levels. | | |
| exchange reaction for cholate | | −0.01 |
| Glycocholate exchange | | −0.01 |
| exchange reaction for beta glucan-glycocholate | | −0.01 |
| complex | | |
| exchange reaction for guar gum-glycocholate | | −0.01 |
| complex | | |
| exchange reaction for psyllium-glycocholic acid | | −0.01 |
| complex | | |
| Binding of guar gums with glycocholate in the | | −0.01 |
| intestinal lumen, reducing serum cholesterol | | |
| levels. | | |
| binding of psyllium with glycocholate in the | | −0.01 |
| intestinal lumen, reducing serum cholesterol | | |
| levels. | | |
| Choloyl-CoA: glycine N-choloyltransferase Bile | BAAT | −0.01 |
| acid biosynthesis/Taurine and hypotaurine | | |
| metabolism EC: 2.3.1.65 | | |
| transport of Phenylalanine by y+LAT1 or y+LAT2 | SLC3A2; | −0.01 |
| with co-transporter of h in small intestine and | SLC7A6; SLC7A7 | |
| kidney | | |
| phosphatidylserine (homo sapiens) exchange | | −0.01 |
| 4-aminobutyrate reversible transport in via | SLC36A1 | −0.01 |
| proton symport | | |
| 4-aminobutyrate reversible transport in via | SLC6A1; | −0.01 |
| sodium symport (1:2) | SLC6A11; | |
| | SLC6A12; | |
| | SLC6A13 | |
| Active transport | | −0.01 |
| calcium/sodium antiporter (1:3), reversible | SLC8A1; | −0.01 |
| | SLC8A2; SLC8A3 | |
| exchange reaction for 3-hydroxy-isovaleryl | | −0.01 |
| carnitine | | |
| fatty acid beta oxidation(C5-->C5OH)m | | −0.01 |
| (3-hydroxyisovalerylcoa-->3- | CPT1A; CPT1B; | −0.01 |
| hydroxyisovalerylcarnitine)c | CPT1C | |
| transport of 3-hydroxyisovalerylcoa from | DBI | −0.01 |
| mitochondria into the cytosol | | |
| transport of 3-hydroxy-isovaleryl carnitine into | | −0.01 |
| extra cellular space | | |
| L-Arginine, NADPH: oxygen oxidoreductase | NOS1; NOS2; | −0.01 |
| (nitric-oxide-forming) Arginine and proline | NOS3 | |
| metabolism EC: 1.14.13.39 | | |
| Nitric Oxide Synthase (NO forming) | NOS1; NOS2; | −0.01 |
| | NOS3 | |
| fatty acid intracellular transport | | −0.01 |
| fructose-bisphosphate aldolase | ALDOART2; | −0.01 |
| | ALDOB; ALDOC | |
| phosphofructokinase | PFKL; PFKM; | −0.01 |
| | PFKP | |
| Sedoheptulose 1,7-bisphosphate D- | ALDOART2; | −0.01 |
| glyceraldehyde-3-phosphate-lyase Carbon | ALDOB; ALDOC | |
| fixation EC: 4.1.2.13 | | |
| transaldolase | TALDO1 | −0.01 |
| UTP: D-fructose-6-phosphate 1- | PFKL | −0.01 |
| phosphotransferase EC: 2.7.1.11 | | |
| RE0512 | EHHADH; HADH; | −0.01 |
| | HADHA; HADHB; | |
| | HSD17B10; | |
| | HSD17B4 | |
| RE1516 | ACADL; ACOX1 | −0.01 |
| RE1517 | ACADL; ACOX1 | −0.01 |
| RE1518 | ACADL; ACOX1 | −0.01 |
| RE1519 | ACADL; ACOX1 | −0.01 |
| RE1520 | ECHS1; | −0.01 |
| | EHHADH; | |
| | HADHA; HADHB | |
| RE1521 | ECHS1; | −0.01 |
| | EHHADH; | |
| | HADHA; HADHB | |
| RE1522 | ECHS1; | −0.01 |
| | EHHADH; | |
| | HADHA; HADHB | |
| RE1523 | ECHS1; | −0.01 |
| | EHHADH; | |
| | HADHA; HADHB | |
| RE1525 | EHHADH; HADH; | −0.01 |
| | HSD17B10; | |
| | HSD17B4 | |
| RE1526 | EHHADH; HADH; | −0.01 |
| | HSD17B10; | |
| | HSD17B4 | |
| RE1527 | EHHADH; HADH; | −0.01 |
| | HSD17B10; | |
| | HSD17B4 | |
| RE1531 | ACAA2; HADHA; | −0.01 |
| | HADHB | |
| RE1532 | ACAA2; HADHA; | −0.01 |
| | HADHB | |
| RE1533 | ACAA2; HADHA; | −0.01 |
| | HADHB | |
| RE1534 | ACAA2; HADHA; | −0.01 |
| | HADHB | |
| RE1573 | ECU; ECI3; | −0.01 |
| | EHHADH | |
| RE3627 | EHHADH; HADH; | −0.01 |
| | HSD17B10; | |
| | HSD17B4 | |
| RE3628 | ECU; ECI3; | −0.01 |
| | EHHADH | |
| ornithine transaminase reversible (m) | OAT | −0.01 |
| Histidine transport (Na, H coupled) | SLC38A3 | −0.01 |
| DM T antigen(g) | | −0.01 |
| Dehydroepiandrosterone transport | | −0.01 |
| Dehydroepiandrosterone sulfate exchange | | −0.01 |
| sulfonated testosterone transport | | −0.01 |
| 5alpha-Dihydrotestosterone sulfate exchange | | −0.01 |
| sodium proton antiporter (H:NA is 1:1) | SLC9A1; | −0.01 |
| | SLC9A2; | |
| | SLC9A3; | |
| | SLC9A4; SLC9A5 | |
| transport of palmitoyl coA into the enterocytes | FABP1 | −0.01 |
| dGTP transport via ATP antiport | SLC25A19 | −0.01 |
| dGTP transport via ADP antiport | SLC25A19 | −0.01 |
| ATP Exporter (ATP-E) TCDB: 9.A.6.1.1 | | −0.01 |
| ATP diphosphohydrolase | ENTPD1; | −0.01 |
| | ENTPD2; | |
| | ENTPD3; | |
| | ENTPD5; | |
| | ENTPD6 | |
| carnitine O-palmitoyltransferase | CPT1A; CPT1B; | −0.01 |
| | CPT1C | |
| carnitine transferase | CPT2 | −0.01 |
| transport into the mitochondria (carnitine) | SLC25A20 | −0.01 |
| Beta oxidation fatty acid | ACADM; ACADS | −0.01 |
| Asparagine transport (Na, H coupled) | SLC38A3; | −0.01 |
| | SLC38A5 | |
| Alanine transport (Na, H coupled) | SLC38A3; | −0.01 |
| | SLC38A5 | |
| Glutamine transport (Na, H coupled) | SLC38A3; | −0.01 |
| | SLC38A5 | |
| Serine transport (Na, H coupled) | SLC38A5 | −0.01 |
| phosphatidylinositol-4,5-bisphosphate 5- | INPP5B; INPP5E; | −0.01 |
| phosphatase | OCRL; SYNJ1; | |
| | SYNJ2 | |
| phosphatidylinositol 4-phosphate 5-kinase | PIKFYVE; | −0.01 |
| | PIP4K2A; | |
| | PIP4K2B; | |
| | PIP4K2C; | |
| | PIP5K1A; | |
| | PIP5K1B; | |
| | PIP5K1C | |
| 2-oxoglutarate dehydrogenase | DLD; DLST; | −0.01 |
| | OGDH; PDHX | |
| 3-Dehydrosphinganine reductase | KDSR | −0.01 |
| Sphinganine 1-phosphate exchange | | −0.01 |
| serine C-palmitoyltransferase | SPTLC1; | −0.01 |
| | SPTLC2; SPTLC3 | |
| sphlp transport | | −0.01 |
| lignoceric acid exchange | | −0.01 |
| fatty-acid--CoA ligase | ACSL1 | −0.01 |
| Beta oxidation of long chain fatty acid | ACAA1B; ACOX1; | −0.01 |
| | EHHADH; | |
| | HSD17B4 | |
| Fatty acid beta oxidation(C24-->C22)x | ACAA1B; ACOX1; | −0.01 |
| | EHHADH; | |
| | HSD17B4 | |
| fatty acid intracellular transport | | −0.01 |
| RE0565 | ELOVL2; | −0.01 |
| | EL0VL4; | |
| | ELOVL5; | |
| | ELOVL6 | |
| RE0566 | | −0.01 |
| RE0567 | | −0.01 |
| RE0568 | RPL14 | −0.01 |
| leukotriene F4 exchange | | −0.01 |
| Transport reaction | | −0.01 |
| betaine-aldehyde dehydrogenase, mitochondrial | | −0.01 |
| choline transport via diffusion (cytosol to | | −0.01 |
| mitochondria) | | |
| choline dehydrogenase (FAD acceptor), | CHDH | −0.01 |
| mitochondrial | | |
| Glycine betaine exchange | | −0.01 |
| Betaine transport (sodium symport) (2:1) | SLC6A12 | −0.01 |
| Glycine betaine transport via diffusion | | −0.01 |
| (mitochondria to cytosol) | | |
| L-Lysine exchange | | −0.01 |
| 5-aminolevulinate synthase | ALAS1; ALAS2; | −0.01 |
| | GCAT | |
| Succinyl-CoA: glycine C-succinyl- | ALAS1; ALAS2 | −0.01 |
| transferase(decarboxylating) EC: 2.3.1.37 | | |
| Succinyl-CoA: glycine C-succinyl- | ALAS1; ALAS2 | −0.01 |
| transferase(decarboxylating) EC: 2.3.1.37 | | |
| 5-L-Glutamyl-L-alanine exchange | | −0.01 |
| Hydrogen peroxide synthesis (NADPH | DUOX1; DUOX2 | −0.01 |
| dependent) | | |
| 3alpha,7alpha-Dihydroxy-5beta-cholestan-26- | ALDH1B1; | −0.01 |
| al: NAD+ oxidoreductase Bile acid biosynthesis | ALDH2; | |
| EC: 1.2.1.3 | ALDH3A2; | |
| | ALDH7A1; | |
| | ALDH9A1 | |
| RE1807 | | −0.01 |
| proline oxidase (NAD), mitochondrial | PRODH | −0.01 |
| pyrroline-5-carboxylate reductase (m) | PYCR1 | −0.01 |
| exchange reaction for cholesterol | | −0.01 |
| L-phenylalanine transport via diffusion | SLC16A10; | −0.01 |
| (extracellular to cytosol) | SLC43A1; | |
| | SLC43A2 | |
| exchange reaction for xylitol | | −0.01 |
| Xylitol transport via passive diffusion | | −0.01 |
| Ammonia exchange | | −0.01 |
| Citrate exchange | | −0.01 |
| Serine/Lysine Na-dependent exchange (Ser in) | SLC3A1; SLC7A7 | −0.01 |
| fatty acid retinol efflux (9-cis) | | −0.01 |
| fatty acid retinol efflux (11-cis) | | −0.01 |
| UDP-GlcNAc: betaGal beta-1,3-N- | B3GNT3 | −0.01 |
| acetylglucosaminyltransferase 3, Golgi apparatus | | |
| UDP-GlcNAc: betaGal beta-1,3-N- | B3GNT3 | −0.01 |
| acetylglucosaminyltransferase 3, Golgi apparatus | | |
| Gal-GlcNAc-Gal globoside (homo sapiens) | | −0.01 |
| exchange | | |
| galacglcgalgbside hs transport | | −0.01 |
| galacglcgalgbside hs intracellular transport | | −0.01 |
| ornithine transaminase reversible (m) | OAT | −0.01 |
| Y+LAT2 Utilized transport | SLC7A6 | −0.01 |
| phosphatidylinositol 3-kinase | HCST; PIK3C2A; | −0.01 |
| | PIK3C2B; | |
| | PIK3C2G; | |
| | PIK3C3; PIK3CA; | |
| | PIK3CB; PIK3CD; | |
| | PIK3CG; PIK3R1; | |
| | PIK3R2; PIK3R3; | |
| | PIK3R5 | |
| RE3270 | | −0.01 |
| Arachidonate 5-lipoxygenase | ALOX5 | −0.01 |
| leukotriene B4 exchange | | −0.01 |
| Leukotriene A-4 hydrolase | LTA4H | −0.01 |
| retinol dehydrogenase (11-cis, NADH) | RDH5 | −0.01 |
| fatty acid beta oxidation(C10: 1-->C10: 2)m | ACADM; ACOX1 | −0.01 |
| reduction(C10: 2-->C10: 1)m | DECR1 | −0.01 |
| fatty acid beta oxidation(C12: 2-->C10: 1)m | ACAA2; ECHS1; | −0.01 |
| | HADH | |
| isomerization(C12: 2)m | ECI1 | −0.01 |
| fatty acid beta oxidation(C14: 2-->C12: 2)m | ACADVL; | −0.01 |
| | HADHA; HADHB | |
| fatty acid beta oxidation(C16: 3-->C16: 4)gm | ACADVL | −0.01 |
| fatty acid beta oxidation(C16: 3-->C14: 2)gm | HADHA; HADHB | −0.01 |
| isomcrization(C16: 3)gm | ECI1 | −0.01 |
| fatty acid beta oxidation(C16: 4-->C16: 3)gm | DECR1 | −0.01 |
| fatty acid beta oxidation(C18: 3-->C16: 3)gm | ACADVL; | −0.01 |
| | HADHA; HADHB | |
| RE2626 | | −0.01 |
| RE3346 | ALDH3A1; | −0.01 |
| | ALDH9A1 | |
| Resistance-Nodulation-Cell Division (RND) | SLCO1B2 | −0.01 |
| TCDB: 2.A.60.1.5 | | |
| transport of L-Tryptophan into the intestinal | SLC6A14 | −0.01 |
| cells by ATBO transporter | | |
| transport of L-Tyrosine into the intestinal cells | SLC6A14 | −0.01 |
| by ATBO transporter | | |
| Glycine secretion via secretory vesicle (ATP | SLC32A1 | −0.01 |
| driven) | | |
| Chitobiose exchange | | −0.01 |
| spermidine monoaldehyde 1 exchange | | −0.01 |
| spermidine monoaldehyde 2 exchange | | −0.01 |
| RE0689 | AOC1 | −0.01 |
| RE0828 | AOC1 | −0.01 |
| phosphoglycerate mutase | BPGM; PGAM1; | −0.01 |
| | PGAM2 | |
| transport of sebacoyl carnitine into extra cellular | | −0.01 |
| space | | |
| transport of Decanoate (n-C10: 0) into the cell by | SLC27A2 | −0.01 |
| diffusion | | |
| transport of Decanoate (n-C10: 0) into the E.R. by | | −0.01 |
| diffusion | | |
| exchange reaction for sebacoyl carnitine | | −0.01 |
| Decanoate (n-C10: 0) exchange | | −0.01 |
| fatty acid omega oxidation(MC10-->w- | CYP4F15 | −0.01 |
| OHMC10)er | | |
| fatty acid omega oxidation(w-OHMC10-->DC10)c | ADH5; ALDH3A2 | −0.01 |
| transport of w-hydroxydecanoic acid by diffusion | | −0.01 |
| sulfate transport via chloride countertransport | SLC26A6 | −0.01 |
| (2:1) | | |
| transport of dCTP into mitochondria | | −0.01 |
| dCTP demand | | −0.01 |
| DM dctp(n) | | −0.01 |
| phosphatidylinositol nuclear transport | | −0.01 |
| (diffusion) | | |
| phosphatidylinositol-4,5-bisphosphate 4- | | −0.01 |
| phosphatase | | |
| phosphatidylinositol-5-phosphate 4-kinase | PI4K2A; PI4KA; | −0.01 |
| | PI4KB | |
| phosphatidylinositol 4-kinase | PI4K2A; PI4KA; | −0.01 |
| | PI4KB | |
| phosphatidylinositol-4-phosphate 4-phosphatase | | −0.01 |
| Beta oxidation of long chain fatty acid | ACAA1B; ACOX1; | −0.01 |
| | EHHADH; | |
| | HSD17B4 | |
| fatty-acyl-CoA synthase (n-C18: 0CoA) | ELOVL2; | −0.01 |
| | ELOVL5; | |
| | ELOVL6; FASN | |
| fatty acid intracellular transport | | −0.01 |
| Demand for folic acid | | −0.01 |
| exchange reaction for Folate | | −0.01 |
| ammonia nuclear transport | | −0.01 |
| ABO blood group (transferase A, alpha 1-3-N- | ABO | −0.01 |
| acetylgalactosaminyltransferase; transferase B, | | |
| alpha 1-3-galactosyltransferase) | | |
| Beta-1,3-galactosyltransferase 5 | B3GALT5 | −0.01 |
| Beta galactosyltransferase | | −0.01 |
| UDP-GlcNAc: betaGal beta-1,3-N- | B3GNT3 | −0.01 |
| acetylglucosaminyltransferase 3, Golgi apparatus | | |
| UDP-GlcNAc: betaGal beta-1,3-N- | B3GNT5 | −0.01 |
| acetylglucosaminyltransferase 5 | | |
| Lea glycolipid exchange | | −0.01 |
| Lacto-N-fucopentaosyl III ceramide exchange | | −0.01 |
| fucosyl galactosylgloboside (homo sapiens) | | −0.01 |
| exchange | | |
| (Gal)3 (Glc)1 (GlcNAc)1 (LFuc)1 (Cer)1 exchange | | −0.01 |
| blood group intracellular transport | | −0.01 |
| blood group intracellular transport | | −0.01 |
| blood group intracellular transport | | −0.01 |
| blood group intracellular transport | | −0.01 |
| fucgalgbside hs transport | | −0.01 |
| fucgalgbside hs intracellular transport | | −0.01 |
| Galactoside 2-alpha-L-fucosyltransferase 1 | FUT1 | −0.01 |
| fucosyltransferase 3 (galactoside 3(4)-L- | | −0.01 |
| fucosyltransferase, Lewis blood group included) | | |
| 1 | | |
| Alpha-(1,3)-fucosyltransferase | FUT9 | −0.01 |
| blood group intracellular transport | | −0.01 |
| blood group intracellular transport | | −0.01 |
| D-lactate transport via proton symport | SLC16A1; | −0.01 |
| | SLC16A3; | |
| | SLC16A7; | |
| | SLC16A8 | |
| D-lactate exchange | | −0.01 |
| L-Lactate exchange | | −0.01 |
| L-Tyrosine exchange | | −0.01 |
| phosphatidylinositol-3,4,5-trisphosphate 3- | PTEN | −0.01 |
| phosphatase | | |
| phosphatidylinositol 4,5-bisphosphate 3-kinase | HCST; PIK3CA; | −0.01 |
| | PIK3CB; PIK3CD; | |
| | PIK3CG; PIK3R1; | |
| | PIK3R2; PIK3R3; | |
| | PIK3R5 | |
| diacylglycerol transport | | −0.01 |
| sodium proton antiporter (H:NA is 1:1) | SLC9A1; | −0.01 |
| | SLC9A2; | |
| | SLC9A3; | |
| | SLC9A4; SLC9A5 | |
| Y+LAT2 Utilized transport | SLC7A6 | −0.01 |
| transport of Glycine into the cell coupled with co- | SLC38A5 | −0.02 |
| transport with Sodium and counter transport | | |
| with proton by SNAT5 transporter. | | |
| Y+LAT2 Utilized transport | SLC7A6 | −0.02 |
| Y+LAT2 Utilized transport | SLC7A6 | −0.02 |
| Y+LAT2 Utilized transport | SLC7A6 | −0.02 |
| omega hydroxy hexadecanoate (n-C16: 0) | | −0.02 |
| exchange | | |
| Fatty acid omega-hydroxylase | CYP4A29 | −0.02 |
| xenobiotic transport | | −0.02 |
| fatty acid intracellular transport | | −0.02 |
| fatty acid intracellular transport | | −0.02 |
| fatty acid intracellular transport | | −0.02 |
| fatty acid transport via diffusion | | −0.02 |
| docosa-4,7,10,13,16-pentaenoic acid (n-6) | | −0.02 |
| exchange | | |
| hexacosanoate (n-C26: 0) exchange | | −0.02 |
| lignoceric acid exchange | | −0.02 |
| tetracosahexaenoic acid, n-3 exchange | | −0.02 |
| tetracosapentaenoic acid, n-6 exchange | | −0.02 |
| tetracosatetraenoic acid n-6 exchange | | −0.02 |
| fatty-acid--CoA ligase | ACSL1 | −0.02 |
| fatty-acid--CoA ligase | ACSL1 | −0.02 |
| fatty-acid--CoA ligase | ACSL1 | −0.02 |
| fatty-acid--CoA ligase | ACSL1 | −0.02 |
| fatty-acid--CoA ligase | ACSL1 | −0.02 |
| fatty-acid--CoA ligase (n-C26: 0) | ACSL1 | −0.02 |
| Beta oxidation of long chain fatty acid | ACAA1B; ACOX1; | −0.02 |
| | EHHADH; | |
| | HSD17B4 | |
| Beta oxidation of long chain fatty acid | ACAA1B; ACOX1; | −0.02 |
| | EHHADH; | |
| | HSD17B4 | |
| Beta oxidation of long chain fatty acid | ACAA1B; ACOX1; | −0.02 |
| | EHHADH; | |
| | HSD17B4 | |
| Beta oxidation of long chain fatty acid | ACAA1B; ACOX1; | −0.02 |
| | EHHADH; | |
| | HSD17B4 | |
| fatty acid intracellular transport | | −0.02 |
| fatty acid transport via diffusion | | −0.02 |
| fatty acid transport via diffusion | | −0.02 |
| fatty acid intracellular transport | | −0.02 |
| fatty acid intracellular transport | | −0.02 |
| fatty acid transport via diffusion | | −0.02 |
| fatty acid intracellular transport | | −0.02 |
| fatty acid transport via diffusion | | −0.02 |
| adrenic acid exchange | | −0.02 |
| fatty-acid--CoA ligase | ACSL1 | −0.02 |
| Y+LAT2 Utilized transport | SLC7A6 | −0.02 |
| Y+LAT2 Utilized transport | SLC7A6 | −0.02 |
| Y+LAT2 Utilized transport | SLC7A6 | −0.02 |
| Retinol exchange | | −0.02 |
| carnitine O-palmitoyltransferase | CPT1A; CPT1B; | −0.02 |
| | CPT1C | |
| carnitine transferase | CPT2 | −0.02 |
| transport into the mitochondria (carnitine) | SLC25A20 | −0.02 |
| Beta oxidation fatty acid | ACADM; ACADS | −0.02 |
| transport of 13-docosenoylcoa into peroxisomes | ABCD1; ABCD2 | −0.02 |
| absorption or uptake of 13-docosenoic acid into | SLC27A1; | −0.02 |
| cells | SLC27A2; | |
| | SLC27A3; | |
| | SLC27A4; | |
| | SLC27A5; | |
| | SLC27A6 | |
| exchange reaction for docosenoic acid (C22: 1) | | −0.02 |
| isomerization(C12: 1)x | ECI3; EHHADH | −0.02 |
| fatty acid beta xoidation(C14: 1-->C12: 1)x | ACAA1B; ACOX1; | −0.02 |
| | EHHADH; | |
| | HSD17B4 | |
| fatty acid beta xoidation(C16: 1-->C14: 1)x | ACAA1B; ACOX1; | −0.02 |
| | EHHADH; | |
| | HSD17B4 | |
| fatty acid beta xoidation(C18: 1-->C16: 1)x | ACAA1B; ACOX1; | −0.02 |
| | EHHADH; | |
| | HSD17B4 | |
| fatty acid beta xoidation(C20: 1-->C18: 1)x | ACAA1B; ACOX1; | −0.02 |
| | EHHADH; | |
| | HSD17B4 | |
| fatty acid beta oxidation(C22: 1-->C20: 1)x | ACAA1B; ACOX1; | −0.02 |
| | EHHADH; | |
| | HSD17B4 | |
| exchange reaction for leucylleucine | | −0.02 |
| hydrolysis of Leucylleucine in the small intestine | LAP3 | −0.02 |
| for cellular uptake | | |
| transport of Leucylleucine by the apical PEPT1 | SLC15A1 | −0.02 |
| amino acid transporters across the brush border | | |
| cells of the enterocytes of the intestine and renal | | |
| cells | | |
| 3MLDA extracellular transport via diffusion | | −0.02 |
| 3-Methylimidazoleacetic acid exchange | | −0.02 |
| 3-Methylimidazole acetaldehyde: NAD+ | ALDH1A3; | −0.02 |
| oxidoreductase | ALDH3A1; | |
| | ALDH3B1; | |
| | ALDH3B3; | |
| | MAOB | |
| N-Methylhistamine: oxygen oxidoreductase | AOC1; AOC2; | −0.02 |
| (deaminating) | AOC3 | |
| S-Adenosyl-L-methionine: histamine N-tele- | HNMT | −0.02 |
| methyltransferase | | |
| Nitric Oxide Synthase (intermediate forming) | NOS1; NOS2; | −0.02 |
| | NOS3 | |
| adenosine facilated transport in cytosol | SLC29A1; | −0.02 |
| | SLC29A2 | |
| Inosine transport (diffusion) | SLC29A1; | −0.02 |
| | SLC29A2 | |
| Facilitated diffusion | | −0.02 |
| Facilitated diffusion | | −0.02 |
| Active transport | | −0.02 |
| Propionate transport, diffusion | | −0.02 |
| retinoic acid transport | | −0.02 |
| L-Leucine exchange | | −0.02 |
| spermidine dialdehyde exchange | | −0.02 |
| RE0690 | AOC1 | −0.02 |
| RE3367 | AOC1 | −0.02 |
| alcohol dehydrogenase (D-1,2-propanediol) | ADH1; ADH4; | −0.02 |
| | ADH5; ADH6A; | |
| | ADH7; ADHFE1; | |
| | ZADH2 | |
| thymidine kinase (ATP: thymidine) | TK1; TK2 | −0.02 |
| 6,7-dihydropteridine reductase | QDPR | −0.02 |
| NADPH: 6,7-dihydropteridine oxidoreductase | QDPR | −0.02 |
| Folate biosynthesis EC: 1.5.1.34 | | |
| kinetensin exchange | | −0.02 |
| fatty acid transport via diffusion | | −0.02 |
| fatty acyl-CoA desaturase (n-C22: 4CoA −> n- | | −0.02 |
| C22: 5CoA) | | |
| docosa-4,7,10,13,16-pentaenoic acid (n-6) | | −0.02 |
| exchange | | |
| fatty-acid--CoA ligase | ACSL1 | −0.02 |
| formaldehyde dehydrogenase | ADH5 | −0.02 |
| formaldehyde dehydrogenase | ADH5 | −0.02 |
| S-(hydroxymethyl)glutathione synthase Methane | | −0.02 |
| metabolism EC: 4.4.1.22 | | |
| S-(hydroxymethyl)glutathione synthase Methane | | −0.02 |
| metabolism EC: 4.4.1.22 | | |
| S-(hydroxymethyl)glutathione dehydrogenase | ADH5 | −0.02 |
| Methane metabolism EC: 1.1.1.284 | | |
| S-(hydroxymethyl)glutathione dehydrogenase | ADH5 | −0.02 |
| Methane metabolism EC: 1.1.1.284 | | |
| RE1709 | | −0.02 |
| RE3050 | | −0.02 |
| folate reductase | DHFR | −0.02 |
| Dihydrofolate: NAD+ oxidoreductase Folate | DHFR | −0.02 |
| biosynthesis EC: 1.5.1.3 | | |
| fructose-bisphosphatase | FBP1; FBP2 | −0.02 |
| lipid, flip-flop intracellular transport | | −0.02 |
| Valine reversible mitochondrial transport | | −0.02 |
| valine transaminase, mitochondiral | BCAT2 | −0.02 |
| L-lactate reversible transport via sodium | SLC5A8 | −0.02 |
| symport | | |
| fatty acid transport via diffusion | | −0.02 |
| clupanodonic acid (docosapentaenoic (n-3)) | | −0.02 |
| exchange | | |
| fatty-acid--CoA ligase | ACSL1 | −0.02 |
| RE1709 | | −0.02 |
| nucleoside-diphosphate kinase (ATP: dGDP), | NME4; NME6 | −0.02 |
| mitochondrial | | |
| RE1796 | HSD3B2 | −0.02 |
| Beta oxidation fatty acid | ACADM; ACADS | −0.02 |
| carnitine O-palmitoyltransferase | CPT1A; CPT1B; | −0.02 |
| | CPT1C | |
| carnitine transferase | CPT2 | −0.02 |
| transport into the mitochondria (carnitine) | SLC25A20 | −0.02 |
| dCTP transport via ADP antiport | SLC25A19 | −0.02 |
| dCTP transport via ATP antiport | SLC25A19 | −0.02 |
| exchange reaction for Riboflavin | | −0.02 |
| L-Arginine exchange | | −0.02 |
| GMP reductase | GMPR; GMPR2 | −0.02 |
| NADH: guanosine-5-phosphate | GMPR; GMPR2 | −0.02 |
| oxidoreductase(deaminating) Purine metabolism | | |
| EC: 1.7.1.7 | | |
| 2-Oxoglutaramate amidohydrolase Glutamate | NIT2 | −0.02 |
| metabolism EC: 3.5.1.3 | | |
| L-Glutamine: pyruvate aminotransferase | | −0.02 |
| Glutamate metabolism EC: 2.6.1.15 | | |
| taurochenodeoxycholate exchange | | −0.02 |
| 4-Aminobutyraldehyde: NAD+ oxidoreductase | ALDH1B1; | −0.02 |
| Urea cycle and metabolism of amino groups | ALDH2; | |
| EC: 1.2.1.3 | ALDH3A2; | |
| | ALDH7A1; | |
| | ALDH9A1 | |
| 4-aminobutanal: NAD+ 1-oxidoreductase; 4- | ALDH1B1; | −0.02 |
| aminobutyraldehyde: NAD+ oxidoreductase Urea | ALDH2; | |
| cycle and metabolism of amino groups/beta- | ALDH3A2; | |
| Alanine metabolism EC: 1.2.1.3 EC: 1.2.1.19 | ALDH7A1; | |
| | ALDH9A1 | |
| 4-Aminobutyraldehyde: NAD+ oxidoreductase | ALDH1B1; | −0.02 |
| Urea cycle and metabolism of amino groups | ALDH2; | |
| EC: 1.2.1.3 | ALDH3A2; | |
| | ALDH7A1; | |
| | ALDH9A1 | |
| 4-aminobutanal: NAD+ 1-oxidoreductase; 4- | ALDH1B1; | −0.02 |
| aminobutyraldehyde: NAD+ oxidoreductase Urea | ALDH2; | |
| cycle and metabolism of amino groups/beta- | ALDH3A2; | |
| Alanine metabolism EC: 1.2.1.3 EC: 1.2.1.19 | ALDH7A1; | |
| | ALDH9A1 | |
| Beta oxidation of fatty acid | ACADM; ACADS | −0.02 |
| Beta oxidation of fatty acid | ACADM; ACADS | −0.02 |
| carnitine O-palmitoyltransferase | CPT1A; CPT1B; | −0.02 |
| | CPT1C | |
| carnitine transferase | CPT2 | −0.02 |
| transport into the mitochondria (carnitine) | SLC25A20 | −0.02 |
| Active transport | | −0.02 |
| 5,6,7,8-Tetrahydrofolate exchange | | −0.02 |
| Leukotriene C4 exchange | | −0.02 |
| digalsgalside hs transport | | −0.02 |
| digalsgalside hs intracellular transport | | −0.02 |
| digalside hs intracellular transport | | −0.02 |
| Digalactosylceramidesulfate exchange | | −0.02 |
| Galactosylceramide sulfotransferase | GAL3ST1 | −0.02 |
| H+/K+ gastric/non-gastric P-ATPase and ABC | ATP4A; ATP4B | −0.02 |
| ATPase | | |
| cytidine facilated transport in cytosol | SLC29A1; | −0.02 |
| | SLC29A2 | |
| exchange reaction for Cytidine | | −0.02 |
| Succinate-semialdehyde: NAD+ oxidoreductase | ALDH5A1 | −0.02 |
| Glutamate metabolism/Tyrosine metabolism/ | | |
| Butanoate metabolism EC: 1.2.1.24 EC: 1.2.1.16 | | |
| Succinate-semialdehyde: NADP+ oxidoreductase | | −0.02 |
| Glutamate metabolism/Tyrosine metabolism/ | | |
| Butanoate metabolism EC: 1.2.1.16 | | |
| N6-(L-1,3-Dicarboxypropyl)-L-lysine: NAD+ | AASS | −0.02 |
| oxidoreductase; N6-(L-1,3-Dicarboxypropyl)-L- | | |
| lysine: NAD+ oxidoreductase (L-glutamate- | | |
| forming) Lysine degradation EC: 1.5.1.9 | | |
| 3,4-Dihydroxymandelaldehyde: NAD+ | ALDH1A3; | −0.02 |
| oxidoreductase Tyrosine metabolism EC: 1.2.1.5 | ALDH3A1; | |
| | ALDH3B1; | |
| | ALDH3B3 | |
| 3,4-Dihydroxymandelaldehyde: NADP+ | ALDH1A3; | −0.02 |
| oxidoreductase Tyrosine metabolism EC: 1.2.1.5 | ALDH3A1; | |
| | ALDH3B1; | |
| | ALDH3B3 | |
| 3-Methoxy-4-hydroxyphenylacetaldehyde: NAD+ | ALDH1A3; | −0.02 |
| oxidoreductase Tyrosine metabolism EC: 1.2.1.5 | ALDH3A1; | |
| | ALDH3B1; | |
| | ALDH3B3 | |
| 3-Methoxy-4- | ALDH1A3; | −0.02 |
| hydroxyphenylacetaldehyde: NADP+ | ALDH3A1; | |
| oxidoreductase Tyrosine metabolism EC: 1.2.1.5 | | |
| | ALDH3B1; | |
| | ALDH3B3 | |
| 3-Methoxy-4- | ALDH1A3; | −0.02 |
| hydroxyphenylglycolaldehyde: NAD+ | ALDH3A1; | |
| oxidoreductase Tyrosine metabolism EC: 1.2.1.5 | ALDH3B1; | |
| | ALDH3B3 | |
| 3-Methoxy-4- | ALDH1A3; | −0.02 |
| hydroxyphenylglycolaldehyde: NADP+ | ALDH3A1; | |
| oxidoreductase Tyrosine metabolism EC: 1.2.1.5 | ALDH3B1; | |
| | ALDH3B3 | |
| saccharopine dehydrogenase (NAD, L-glutamate | AASS | −0.02 |
| forming), mitochondrial | | |
| Active transport | | −0.02 |
| Transport reaction | | −0.02 |
| tetrahydrofolate transport via anion exchange | SLC19A1 | −0.02 |
| AKG transporter, peroxisome | | −0.02 |
| aldehyde dehydrogenase (pristanal, NAD) | ALDH3A2 | −0.02 |
| 2,6 dimethylheptanoly CoA carnitine transferase | | −0.02 |
| 2,6 dimethylheptanoyl crn transport | | −0.02 |
| 2,6 dimethylheptanoyl carnitine transport | | −0.02 |
| DMNONCRNCPT2 | | −0.02 |
| 2,6 dimethylheptanoyl carnitine exchange | | −0.02 |
| phytanic acid exchange | | −0.02 |
| fatty-acid--CoA ligase | SLC27A2 | −0.02 |
| Beta oxidation of long chain fatty acid | ACADM | −0.02 |
| fatty acid alpha oxidation(2-Hydroxyphytanoyl- | ALDH3A2; | −0.02 |
| CoA)(nadp)x | HACL1 | |
| fatty acid alpha oxidation(2-Hydroxyphytanoyl- | ALDH3A2; | −0.02 |
| CoA)(nad)x | HACL1 | |
| formyl coa hydrolase | | −0.02 |
| formyl coa transport | | −0.02 |
| 2-hydroxyphytanoyl-CoA lyase | | −0.02 |
| peroxisomal lumped long chain fatty acid | ACAA1B; ACOX3; | −0.02 |
| oxidation | EHHADH; | |
| | HSD17B4 | |
| Phytanoyl-CoA dioxygenase, peroxisomal | PHYH | −0.02 |
| fatty acid transport via diffusion | | −0.02 |
| pristanal peroxisomal transport | | −0.02 |
| prist peroxisomal transport | | −0.02 |
| Sterol carrier protein 2 | SCP2 | −0.02 |
| 3-mercaptolactate-cysteine disulfide exchange | | −0.02 |
| 3-mercaptolactate: cysteine reductase | | −0.02 |
| 3-Mercaptolactate: NAD+ oxidoreductase | LDHA; | −0.02 |
| | LDHAL6B; | |
| | LDHB; LDHC; | |
| | UEVLD | |
| 3-mercaptolactate-cysteine disulfide transport, | | −0.02 |
| extracellular | | |
| UDPglucose pyrophosphohydrolase | ENPP1; ENPP2; | −0.02 |
| | ENPP3 | |
| lipid, flip-flop intracellular transport | | −0.02 |
| glucose 6-phosphate endoplasmic reticular | SLC37A4 | −0.02 |
| transport via diffusion | | |
| phosphate transport, endoplasmic reticulum | SLC37A4 | −0.02 |
| phosphate transport, endoplasmic reticulum | SLC37A4 | −0.02 |
| Diphosphate transporter, endoplasmic reticulum | | −0.02 |
| Diphosphate transporter, endoplasmic reticulum | | −0.02 |
| Resistance-Nodulation-Cell Division (RND) | CP | −0.02 |
| TCDB: 2.A.65.1.1 | | |
| Resistance-Nodulation-Cell Division (RND) | CP | −0.02 |
| TCDB: 2.A.65.1.1 | | |
| Utilized transport | | −0.02 |
| Utilized transport | | −0.02 |
| Utilized transport | | −0.02 |
| Utilized transport | | −0.02 |
| lipid, flip-flop intracellular transport | | −0.02 |
| Major Facilitator(MFS) TCDB: 2.A.1.4.5 | SLC37A4 | −0.02 |
| ribokinase | RBKS | −0.02 |
| RE2626 | | −0.02 |
| RE3346 | ALDH3A1; | −0.02 |
| | ALDH9A1 | |
| adenosine kinase | ADK | −0.02 |
| 5′-nucleotidase (AMP) | NT5C; NT5C1A; | −0.02 |
| | NT5C1B; NT5C2; | |
| | NT5C3 | |
| L-histidine transport in via sodium symport | SLC38A2; | −0.02 |
| | SLC6A14 | |
| phosphatidylinositol 4-phosphate 5-kinase, | | −0.02 |
| nuclear | | |
| phosphatidylinositol-5-phosphate 4-kinase, | | −0.02 |
| nuclear | | |
| phosphatidylinositol 5-kinase, nuclear | | −0.02 |
| acetyl-CoA synthetase | AACS; ACSS2 | −0.02 |
| N-acetylglucosamine-6-phosphate synthase | GNPNAT1 | −0.02 |
| N-acetylglucosamine-6-phosphate deacetylase | AMDHD2 | −0.02 |
| RE1901 | | −0.02 |
| timnodonic acid exchange | | −0.02 |
| fatty-acid--CoA ligase | ACSL1 | −0.02 |
| fatty acid transport via diffusion | | −0.02 |
| pyruvate dehydrogenase | DLAT; DLD; | −0.02 |
| | PDHA1; PDHA2; | |
| | PDHB; PDHX | |
| Resistance-Nodulation-Cell Division (RND) | SLCO2A1 | −0.02 |
| TCDB: 2.A.60.1.2 | | |
| CTP synthase (NH3) | CTPS; CTPS2 | −0.02 |
| Transport of octadecenoyl coA into the | FABP1 | −0.02 |
| enterocytes | | |
| ATP-Citrate lyase | ACLY | −0.02 |
| transport of 3-hydroxytetradeca dienoyl coa | DB1 | −0.02 |
| from mitochondria into cytosol | | |
| production of 3-hydroxytetradeca dienoyl | CPT1A; CPT1B; | −0.02 |
| carnitine | CPT1C | |
| excretion of C14: 2-OH | | −0.02 |
| exchange reaction for 3-hydroxy | | −0.02 |
| trans5,8tetradecadienoyl carnitine | | |
| fatty acid beta oxidation(C14: 2-->C14: 2OH)m | ACADVL; | −0.02 |
| | HADHA | |
| fatty acid beta oxidation trans(C16: 2-->C14: 2)m | ACADVL; | −0.02 |
| | HADHA; HADHB | |
| L-phenylalanine transport in via sodium symport | ACE2; SLC38A1; | −0.02 |
| | SLC38A2; | |
| | SLC38A4; | |
| | SLC6A14; | |
| | SLC6A19; | |
| | SLC7A1; | |
| | SLC7A2; | |
| | SLC7A3; | |
| | TMEM27 | |
| transport of L-Lysine into the intestinal cells by | SLC6A14 | −0.02 |
| ATB0 transporter | | |
| transport of L-Arginine into the intestinal cells by | SLC6A14 | −0.02 |
| ATB0 transporter | | |
| Phosphatidylcholine (homo sapiens) exchange | | −0.02 |
| UDP-GlcNAc: betaGal beta-1,3-N- | B3GNT3 | −0.02 |
| acetylglucosaminyltransferase 3, Golgi apparatus | | |
| UDP-GlcNAc: betaGal beta-1,3-N- | B3GNT3 | −0.02 |
| acetylglucosaminyltransferase 3, Golgi apparatus | | |
| UDP-GlcNAc: betaGal beta-1,3-N- | B3GNT3 | −0.02 |
| acetylglucosaminyltransferase 3, Golgi apparatus | | |
| Gal-Gal-Gal-Gal-Gal-Glc-Cer (homo sapiens) | | −0.02 |
| exchange | | |
| galgalgalthcrm hs transport | | −0.02 |
| galgalgalthcrm hs intracellular transport | | −0.02 |
| DM dsT antigen(g) | | −0.02 |
| alpha-N-acetylgalactosaminide alpha-2,6- | ST6GALNAC1 | −0.02 |
| sialyltransferase | | |
| beta-galactoside alpha-2,3-sialyltransferase (T | ST3GAL1 | −0.02 |
| antigen) | | |
| trans-4-Hydroxy-L-proline: NAD+ 5- | PYCR1; PYCR2 | −0.02 |
| oxidoreductase Arginine and proline metabolism | | |
| EC: 1.5.1.2 | | |
| trans-4-Hydroxy-L-proline: NADP+ 5- | PYCR1; PYCR2 | −0.02 |
| oxidoreductase Arginine and proline metabolism | | |
| EC: 1.5.1.2 | | |
| 3-hydroxybutyryl-CoA dehydratase, | ECHDC2 | −0.03 |
| mitochondrial | | |
| transport of (R)-3-hydroxybutanoyl-CoA into | DBI | −0.03 |
| cytosol | | |
| transport of 3-hydroxy butyryl carnitine into | | −0.03 |
| extra cellular space | | |
| production of 3-hydroxyhytyryl carnitinec | CPT1A; CPT1B; | −0.03 |
| | CPT1C | |
| exchange reaction for 3-hydroxy butyryl | | −0.03 |
| carnitine | | |
| N-acetylglucosaminylphosphatidylinositol | PIGL | −0.03 |
| deacetylase | | |
| B mannosyltransferase, endoplasmic reticulum | PIGB | −0.03 |
| B mannosyltransferase, endoplasmic reticulum | PIGB | −0.03 |
| DM dem2emgacpail prot hs(r) | | −0.03 |
| DM gpi sig(er) | | −0.03 |
| DM mem2emgacpail prot hs(r) | | −0.03 |
| glucosaminyl-acylphosphatidylinositol ER | | −0.03 |
| flippase | | |
| glucosaminylphosphatidyl inositol | PIGW | −0.03 |
| acetyltransferase | | |
| glycophosphatidylinositol (GPI) deacylase, | PGAP1 | −0.03 |
| endoplasmic reticulum | | |
| GlcN-acylPI mannosyltransferase, endoplasmic | PIGM; PIGX | −0.03 |
| reticulum | | |
| GlcN-acylPI mannosyltransferase, endoplasmic | PIGM; PIGX | −0.03 |
| reticulum | | |
| H2 phosphoethanolaminyl transferase, | PIGN | −0.03 |
| endoplasmic reticulum | | |
| H2 mannosyltransferase, endoplasmic reticulum | PIGV | −0.03 |
| H2 mannosyltransferase, endoplasmic reticulum | PIGV | −0.03 |
| H3 phosphoethanolaminyl transferase, | PIGN | −0.03 |
| endoplasmic reticulum | | |
| H3 mannosyltransferase, endoplasmic reticulum | PIGB | −0.03 |
| H3 mannosyltransferase, endoplasmic reticulum | PIGB | −0.03 |
| H4 phosphoethanolaminyl transferase, | PIGF; PIGO | −0.03 |
| endoplasmic reticulum | | |
| H4 phosphoethanolaminyl transferase, | PIGN | −0.03 |
| endoplasmic reticulum | | |
| H5 mannosyltransferase, endoplasmic reticulum | PIGV | −0.03 |
| H5 mannosyltransferase, endoplasmic reticulum | PIGV | −0.03 |
| H6′ phosphoethanolaminyl transferase, | PIGN | −0.03 |
| endoplasmic reticulum | | |
| H6 phosphoethanolaminyl transferase, | PIGF; PIGO | −0.03 |
| endoplasmic reticulum | | |
| H6 mannosyltransferase, endoplasmic reticulum | PIGZ | −0.03 |
| H6 mannosyltransferase, endoplasmic reticulum | PIGZ | −0.03 |
| H7′ transamidase, endoplasmic reticulum | GPAA1; PIGK; | −0.03 |
| | PIGS; PIGT; PIGU | |
| H7 mannosyltransferase, endoplasmic reticulum | PIGZ | −0.03 |
| H7 mannosyltransferase, endoplasmic reticulum | PIGZ | −0.03 |
| M4B transamidase, endoplasmic reticulum | GPAA1; PIGK; | −0.03 |
| | PIGS; PIGT; PIGU | |
| M4C phosphoethanolaminyl transferase, | PIGF; PIGO | −0.03 |
| endoplasmic reticulum | | |
| phosphatidylinositol N- | DPM2; PIGA; | −0.03 |
| acetylglucosaminyltransferase | PIGO; PIGH; | |
| | PIGP; PIGQ | |
| glycophosphatidylinositol (GPI)-anchored | | −0.03 |
| protein precursor sink | | |
| Y+LAT2 Utilized transport | SLC7A6 | −0.03 |
| Y+LAT2 Utilized transport | SLC7A6 | −0.03 |
| Y+LAT2 Utilized transport | SLC7A6 | −0.03 |
| Y+LAT2 Utilized transport | SLC7A6 | −0.03 |
| glucuronidated compound transport | | −0.03 |
| ATP-binding Cassette (ABC) TCDB: 3.A.1.208.9 | ABCC3 | −0.03 |
| ATP-binding Cassette (ABC) TCDB: 3.A.1.204.5 | ABCG5; ABCG8 | −0.03 |
| Active transport | | −0.03 |
| Active transport | | −0.03 |
| lipid, flip-flop intracellular transport | | −0.03 |
| lipid, flip-flop intracellular transport | | −0.03 |
| insosine kinase | | −0.03 |
| ATP: Sedoheptulose 7-phosphate 1- | PFKL | −0.03 |
| phosphotransferase EC: 2.7.1.11 | | |
| UTP: Sedoheptulose 7-phosphate 1- | PFKL | −0.03 |
| phosphotransferase EC: 2.7.1.11 | | |
| nucleoside-diphosphate kinase (ATP: IDP) | GM20390; | −0.03 |
| | NME2; NME3; | |
| | NME6; NME7 | |
| nucleoside-diphosphate kinase (ATP: IDP) | GM20390; | −0.03 |
| | NME2; NME3; | |
| | NME6; NME7 | |
| ATP: Sedoheptulose 7-phosphate 1- | PFKL | −0.03 |
| phosphotransferase EC: 2.7.1.11 | | |
| UTP: Sedoheptulose 7-phosphate 1- | PFKL | −0.03 |
| phosphotransferase EC: 2.7.1.11 | | |
| CTP: D-Tagatose 6-phosphate 1- | PFKL | −0.03 |
| phosphotransferase Galactose metabolism | | |
| EC: 2.7.1.11 | | |
| CTP: D-Tagatose 6-phosphate 1- | PFKL | −0.03 |
| phosphotransferase Galactose metabolism | | |
| EC: 2.7.1.11 | | |
| ITP: D-Tagatose 6-phosphate 1- | PFKL | −0.03 |
| phosphotransferase Galactose metabolism | | |
| EC: 2.7.1.11 | | |
| ITP: D-Tagatose 6-phosphate 1- | PFKL | −0.03 |
| phosphotransferase Galactose metabolism | | |
| EC: 2.7.1.11 | | |
| Beta-1,3-galactosyltransferase 4 | B3GALT4 | −0.03 |
| Beta-1,3-galactosyltransferase 4 | B3GALT4 | −0.03 |
| Beta-1,3-galactosyltransferase 4 | B3GALT4 | −0.03 |
| GP1c (homo sapiens) exchange | | −0.03 |
| GP1c alpha (homo sapiens) exchange | | −0.03 |
| GQ1b (homo sapiens) exchange | | −0.03 |
| GQ1balpha (homo sapiens) exchange | | −0.03 |
| GT1a (homo sapiens) exchange | | −0.03 |
| Beta-1,4 N-acetylgalactosaminyltransferase | B4GALNT1 | −0.03 |
| Beta-1,4 N-acetylgalactosaminyltransferase | B4GALNT1 | −0.03 |
| Beta-1,4 N-acetylgalactosaminyltransferase | B4GALNT1 | −0.03 |
| gp1calpha hs transport | | −0.03 |
| gp1calpha hs intracellular transport | | −0.03 |
| gp1c hs transport | | −0.03 |
| gp1c hs intracellular transport | | −0.03 |
| gq1balpha hs transport | | −0.03 |
| gq1balpha hs intracellular transport | | −0.03 |
| gq1b hs transport | | −0.03 |
| gq1b hs intracellular transport | | −0.03 |
| gt1a hs transport | | −0.03 |
| gt1a hs intracellular transport | | −0.03 |
| CMP-N-acetylneuraminate-beta-galactosamide- | ST3GAL2 | −0.03 |
| alpha-2,3-sialyltransferase, Golgi apparatus | | |
| CMP-N-acetylneuraminate-beta-galactosamide- | ST3GAL2 | −0.03 |
| alpha-2,3-sialyltransferase, Golgi apparatus | | |
| Alpha-N-acetylgalactosaminide alpha-2,6- | ST6GALNAC2 | −0.03 |
| sialyltransferase 2 | | |
| Alpha-N-acetylgalactosaminide alpha-2,6- | ST6GALNAC2 | −0.03 |
| sialyltransferase 2 | | |
| Alpha-N-acetylgalactosaminide alpha-2,6- | ST6GALNAC2 | −0.03 |
| sialyltransferase 2 | | |
| Alpha-N-acetylgalactosaminide alpha-2,6- | ST6GALNAC2 | −0.03 |
| sialyltransferase 2 | | |
| Alpha-N-acetylgalactosaminide alpha-2,6- | ST6GALNAC3; | −0.03 |
| sialyltransferase 3 | ST6GALNAC5; | |
| | ST6GALNAC6 | |
| sialytransferase 7 ((alpha-N-acetylneuraminyl | ST6GALNAC6 | −0.03 |
| 2,3-betagalactosyl-1,3)-N-acetyl galactosaminide | | |
| alpha-2,6-sialytransferase) F | | |
| Beta-galactoside alpha-2,3-sialyltransferase | ST8SIA5 | −0.03 |
| Beta-galactoside alpha-2,3-sialyltransferase | ST8SIA5 | −0.03 |
| Beta-galactoside alpha-2,3-sialyltransferase | ST8SIA5 | −0.03 |
| fatty acyl-CoA desaturase (n-C18: 2CoA −> n- | FADS2 | −0.03 |
| C18: 3CoA) | | |
| fatty acyl-CoA desaturase (n-C24: 5CoA −> n- | FADS2 | −0.03 |
| C24: 6CoA) | | |
| stearidonic acid exchange | | −0.03 |
| tetracosahexaenoic acid, n-3 exchange | | −0.03 |
| tetracosapentaenoic acid, n-3 exchange | | −0.03 |
| vaccenic acid exchange | | −0.03 |
| fatty-acid--CoA ligase | ACSL1 | −0.03 |
| fatty-acid--CoA ligase | ACSL1 | −0.03 |
| fatty-acid--CoA ligase | ACSL1 | −0.03 |
| fatty-acid--CoA ligase | ACSL1 | −0.03 |
| fatty-acid--CoA ligase | ACSL1 | −0.03 |
| fatty acid transport via diffusion | | −0.03 |
| fatty acid transport via diffusion | | −0.03 |
| fatty acid transport via diffusion | | −0.03 |
| fatty acid transport via diffusion | | −0.03 |
| Xanthurenic acid exchange | | −0.03 |
| Transport reaction | | −0.03 |
| ATP: pantothenate 4-phosphotransferase | PANK1; PANK2; | −0.03 |
| Pantothenate and CoA biosynthesis EC: 2.7.1.33 | PANK3; PANK4 | |
| RE3272 | | −0.03 |
| IV3-a-NeuAc,III3-a-Fuc-nLc4Cer exchange | | −0.03 |
| IV3-a-Neu5Ac,III4-a-Fuc-Lc4Cer exchange | | −0.03 |
| blood group intracellular transport | | −0.03 |
| blood group intracellular transport | | −0.03 |
| blood group intracellular transport | | −0.03 |
| blood group intracellular transport | | −0.03 |
| fucosyltransferase 3 (galactoside 3(4)-L- | | −0.03 |
| fucosyltransferase, Lewis blood group included) | | |
| 1 | | |
| Alpha-(1,3)-fucosyltransferase | FUT9 | −0.03 |
| ST3 beta-galactoside alpha-2,3-sialyltransferase | ST3GAL3 | −0.03 |
| 3 | | |
| Type 2 lactosamine alpha-2,3-sialyltransferase | ST3GAL6 | −0.03 |
| fatty acyl-CoA synthase (n-C8: 0CoA), lumped | FASN | −0.03 |
| reaction | | |
| Major Facilitator(MFS) TCDB: 2.A.1.14.6 | SLC17A1 | −0.03 |
| adrenic acid exchange | | −0.03 |
| fatty-acid--CoA ligase | ACSL1 | −0.03 |
| Generic human biomass reaction | | −0.03 |
| cardiolipin synthase (homo sapiens) | CRLS1 | −0.03 |
| phosphatidylglycerol transport | | −0.03 |
| 4-Aminobutanoate exchange | | −0.03 |
| transport of beta alanine by PAT1 in renal and | SLC36A1 | −0.03 |
| intestinal cells | | |
| NACHORCTL3le | SLC22A13B-PS | −0.03 |
| D-proline transport, extracellular | | −0.03 |
| D-proline reversible transport via proton | SLC36A1 | −0.03 |
| symport | | |
| Zinc (Zn2+)-Iron (Fe2+) Permease (ZIP), | SLC11A1 | −0.03 |
| TCDB: 2.A.55.2.3 | | |
| Cation Diffusion Facilitator (CDF) TCDB: 2.A.4.2.3 | SLC30A1 | −0.03 |
| ribose transport via diffusion | | −0.03 |
| ribose transport in via proton symporter | | −0.03 |
| methylenetetrahydrofolate dehydrogenase | MTHFD2 | −0.03 |
| (NAD), mitochondrial | | |
| methylenetetrahydrofolate dehydrogenase | MTHFD1; | −0.03 |
| (NADP), mitochondrial | MTHFD1L; | |
| | MTHFD2 | |
| keratan sulfate I, degradation product 1 exchange | | −0.03 |
| keratan sulfate I exchange | | −0.03 |
| alpha-fucosidase, extracellular | FUCA2 | −0.03 |
| Platelet-activating factor acetylhydrolase | PAFAH1B1; | −0.03 |
| | PAFAH1B2; | |
| | PAFAH1B3; | |
| | PAFAH2 | |
| alkyl glycerol phosphocholine acetyl transferase | | −0.03 |
| acetyl-CoA hydrolase | ACOT12 | −0.03 |
| acetyl-CoA synthase (propionate) | AACS; ACSS2 | −0.03 |
| RE3629 | | −0.03 |
| RE3630 | | −0.03 |
| EC: 1.3.3.6 | ACOX1; ACOX3 | −0.03 |
| EC: 1.3.3.6 | ACOX1; ACOX3 | −0.03 |
| EC: 1.3.3.6 | ACOX1; ACOX3 | −0.03 |
| EC: 1.3.3.6 | ACOX1; ACOX3 | −0.03 |
| EC: 1.3.3.6 | ACOX1; ACOX3 | −0.03 |
| exchange reaction for ribose | | −0.03 |
| ribose transport via diffusion | | −0.03 |
| Dephospho-CoA nucleotidohydrolase | ENPP1; ENPP3 | −0.03 |
| Pantothenate and CoA biosynthesis EC: 3.6.1.9 | | |
| L-tryptophan transport | SLC16A10; | −0.03 |
| | SLC36A4 | |
| L-tyrosine transport | SLC16A10 | −0.03 |
| GDP exchange | | −0.03 |
| GDP exchange | | −0.03 |
| GMP exchange | | −0.03 |
| GTP exchange | | −0.03 |
| UDP exchange | | −0.03 |
| UTP exchange | | −0.03 |
| nucleoside-diphosphatase (GDP), extracellular | CANT1; | −0.03 |
| | ENTPD1; | |
| | ENTPD3; | |
| | ENTPD8 | |
| nucleoside-triphosphatase (GTP) | CANT1; | −0.03 |
| | ENTPD1; | |
| | ENTPD3; | |
| | ENTPD8 | |
| lipid, flip-flop intracellular transport | | −0.03 |
| Isocitrate dehydrogenase (NADP+) | IDH2 | −0.03 |
| 3-hydroxyacyl-CoA dehydratase (3- | AUH; ECHS1; | −0.03 |
| hydroxybutanoyl-CoA) (mitochondria) | HADHA; HADHB | |
| 3-hydroxyacyl-CoA dehydrogenase (acetoacetyl- | HADH; HADHA; | −0.03 |
| CoA) (mitochondria) | HADHB; | |
| | HSD17B10 | |
| transport of glutaryl carnitine into the extra | | −0.03 |
| cellular fluid | | |
| exchange reaction for glutaryl carnitine | | −0.03 |
| production of glutaryl carnitine | CPT1A; CPT1B; | −0.03 |
| | CPT1C | |
| transport of glutaryl-CoA(5-) from mitochondria | DBl | −0.03 |
| into cytosol | | |
| Resistance-Nodulation-Cell Division (RND) | SLCO1A1 | −0.03 |
| TCDB: 2.A.60.1.14 | | |
| transport of Leucine by y+LAT1 or y+LAT2 with | SLC3A2; | −0.03 |
| co-transporter of h in small intestine and kidney | SLC7A6; SLC7A7 | |
| arachidyl coenzyme A exchange | | −0.03 |
| 3-oxodocosanoyl-CoA exchange | | −0.03 |
| exchange reaction for coa | | −0.03 |
| Exchange of Uroporphyrinogenl | | −0.03 |
| Exchange of Uroporphyrinogenl | | −0.03 |
| Exchange of Coproporphyrinogenl | | −0.03 |
| Exchange of Coproporphyrinogenl | | −0.03 |
| malonyl-CoA(4-) exchange | | −0.03 |
| LAPCOAe | | −0.03 |
| Uroporphyrinogen I carboxy-lyase Porphyrin and | UROD | −0.03 |
| chlorophyll metabolism EC: 4.1.1.37 | | |
| Uroporphyrinogen I carboxy-lyase Porphyrin and | UROD | −0.03 |
| chlorophyll metabolism EC: 4.1.1.37 | | |
| RE0569 | LOXL2 | −0.03 |
| RE2404 | UGT1A1 | −0.03 |
| RE2405 | UGT1A1 | −0.03 |
| RE2541 | KL | −0.03 |
| RE3381 | KL | −0.03 |
| UDPglucuronate uridine-diphosphohydrolase | | −0.03 |
| UDPglucose 6-dehydrogenase | UGDH | −0.03 |
| exchange reaction for Glycine | | −0.03 |
| phosphatidylinositol 4,5-bisphosphate nuclear | | −0.03 |
| transport (diffusion) | | |
| RE1447 | | −0.03 |
| RE1448 | | −0.03 |
| phosphatidylinositol 4-phosphate nuclear | | −0.03 |
| transport (diffusion) | | |
| Acetoacetyl-CoA: acetate CoA-transferase | AACS | −0.03 |
| aldo-keto reductase family 1, member C4 | AKR1C6 | −0.03 |
| (chlordecone reductase; 3-alpha hydroxysteroid | | |
| dehydrogenase, type I; dihydrodiol | | |
| dehydrogenase 4) | | |
| aldo-keto reductase family 1, member C4 | AKR1C6 | −0.03 |
| (chlordecone reductase; 3-alpha hydroxysteroid | | |
| dehydrogenase, type I; dihydrodiol | | |
| dehydrogenase 4) | | |
| dihydrothymin dehydrogenase (NADP) | DPYD | −0.03 |
| CMP-N-acetylneuraminate, ferrocytochrome- | | −0.03 |
| b5: oxygen oxidoreductase (N-acetyl- | | |
| hydroxylating) Aminosugars metabolism | | |
| EC: 1.14.18.2 | | |
| cytidine monophospho-N-acetylneuraminic acid | | −0.03 |
| hydroxylase EC: 1.14.18.2 | | |
| 5,6-Dihydrothymine: NAD+ oxidoreductase | | −0.03 |
| Pyrimidine metabolism EC: 1.3.1.1 | | |
| 3alpha,7alpha-Dihydroxy-5beta- | AKR1C6 | −0.03 |
| cholestane: NADP+ oxidoreductase (B-specific); | | |
| 3alpha,7alpha-Dihydroxy-5beta- | | |
| cholestane: NADP+ oxidoreductase Bile acid | | |
| biosynthesis EC: 1.1.1.50 | | |
| 3alpha,7alpha,12alpha-Trihydroxy-5beta- | AKR1C6 | −0.03 |
| cholestane: NADP+ oxidoreductase (B-specific); | | |
| 3alpha,7alpha,12alpha-Trihydroxy-5beta- | | |
| cholestane: NADP+ oxidoreductase Bile acid | | |
| biosynthesis EC: 1.1.1.50 | | |
| phosphatidylinositol synthase (Homo sapiens) | CDIPT | −0.03 |
| phosphatidate cytidylyltransferase | CDS1; CDS2 | −0.03 |
| RE3273 | PLD2 | −0.03 |
| Uridine triphosphate pyrophosphohydrolase | ITPA | −0.03 |
| Pyrimidine metabolism EC: 3.6.1.19 | | |
| 5′-nucleotidase (UMP), extracellular | NT5E | −0.03 |
| uridine facilated transport in cytosol | SLC29A1; | −0.03 |
| | SLC29A2 | |
| Y+LAT2 Utilized transport | SLC7A6 | −0.03 |
| Y+LAT2 Utilized transport | SLC7A6 | −0.03 |
| Y+LAT2 Utilized transport | SLC7A6 | −0.03 |
| Adenine exchange | | −0.03 |
| AMP exchange | | −0.03 |
| Exchange of 5-O-phosphonato-alpha-D- | | −0.03 |
| ribofuranosyl diphosphate(5-) | | |
| AMP: pyrophosphate phosphoribosyltransferase | APRT; HPRT | −0.03 |
| Purine metabolism EC: 2.4.2.7 | | |
| Y+LAT2 Utilized transport | SLC7A6 | −0.03 |
| Y+LAT2 Utilized transport | SLC7A6 | −0.03 |
| acetyl-coa transport | SLC33A1 | −0.03 |
| N-Acetylneuraminate lyase (reversible) | | −0.03 |
| CMP-Sia Golgi transport via CMP antiport | SLC35A1 | −0.03 |
| CoA transport in Golgi via diffusion | | −0.03 |
| 9-O-Acetylated GD3 (homo sapiens) exchange | | −0.03 |
| 9-O-Acetylated GT3 (homo sapiens) exchange | | −0.03 |
| Ganglioside O-acetylation | SLC33A1 | −0.03 |
| Ganglioside O-acetylation | SLC33A1 | −0.03 |
| oagd3 hs transport | | −0.03 |
| oagd3 hs intracellular transport | | −0.03 |
| oagt3 hs transport | | −0.03 |
| oagt3 hs intracellular transport | | −0.03 |
| N-acetylneuraminate, ferrocytochrome- | | −0.03 |
| b5: oxygen oxidoreductase (N-acetyl- | | |
| hydroxylating) Aminosugars metabolism | | |
| EC: 1.14.18.2 | | |
| CTP: N-acylneuraminate cytidylyltransferase | CMAS | −0.03 |
| Aniinosugars metabolism EC: 2.7.7.43 | | |
| Lactosylceramide alpha-2,3-sialyltransferase, | ST3GAL5 | −0.03 |
| Golgi apparatus | | |
| Beta-galactoside alpha-2,3-sialyltransferase | ST8SIA1 | −0.03 |
| Beta-galactoside alpha-2,3-sialyltransferase | ST8SIA5 | −0.03 |
| Alanine transport (Na, H coupled) | SLC38A3; | −0.03 |
| | SLC38A5 | |
| Serine transport (Na, H coupled) | SLC38A5 | −0.03 |
| RE2759 | AMACR | −0.03 |
| RE2759 | AMACR | −0.03 |
| RE3074 | ACSL1; ACSL3; | −0.03 |
| | ACSL4; ACSL5; | |
| | ACSL6; SLC27A2 | |
| RE3074 | ACSL1; ACSL3; | −0.03 |
| | ACSL4; ACSL5; | |
| | ACSL6; SLC27A2 | |
| RE3079 | SLC27A2 | −0.03 |
| RE3079 | SLC27A2 | −0.03 |
| acyl-Coenzyme A oxidase 2, branched chain | ACOX2 | −0.03 |
| FADH2 transporter, peroxisomal | | −0.03 |
| FAD transporter, peroxisomal | | −0.03 |
| hydroxysteroid (17-beta) dehydrogenase 4 | HSD17B4 | −0.03 |
| 2-hydroxybutyrate cotransport with proton | SLC16A1; | −0.03 |
| | SLC16A3; | |
| | SLC16A7 | |
| acetoacetate transport via proton symport | SLC16A1; | −0.03 |
| | SLC16A7 | |
| acetate reversible transport via proton symport | | −0.03 |
| (R)-3-Hydroxybutanoate transport via H+ | | −0.03 |
| symport | | |
| Butyrate transport via proton symport, | SLC16A1 | −0.03 |
| reversible | | |
| pyruvate reversible transport via proton symport | SLC16A1; | −0.03 |
| | SLC16A3; | |
| | SLC16A7 | |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| 2-hydroxybutyrate cotransport with proton | SLC16A1; | −0.03 |
| | SLC16A3; | |
| | SLC16A7 | |
| acetoacetate transport via proton symport | SLC16A1; | −0.03 |
| | SLC16A7 | |
| (R)-3-Hydroxybutanoate transport via H+ | | −0.03 |
| symport | | |
| Butyrate transport via proton symport, | SLC16A1 | −0.03 |
| reversible | | |
| pyruvate reversible transport via proton symport | SLC16A1; | −0.03 |
| | SLC16A3; | |
| | SLC16A7 | |
| L-lactate reversible transport via proton symport | BSG; SLC16A1; | −0.03 |
| | SLC16A3; | |
| | SLC16A7; | |
| | SLC16A8 | |
| acetate reversible transport via proton symport | | −0.03 |
| L-lactate reversible transport via proton symport | BSG; SLC16A1; | −0.03 |
| | SLC16A3; | |
| | SLC16A7; | |
| | SLC16A8 | |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| glycerol-3-phosphate dehydrogenase (NAD) | GPD1 | −0.03 |
| glycerol-3-phosphate dehydrogenase (NAD) | GPD1 | −0.03 |
| transport of NAD into peroxisome | | −0.03 |
| transport of NAD into peroxisome | | −0.03 |
| pyruvate peroxisomal transport via proton | SLC16A1; | −0.03 |
| symport | SLC16A7 | |
| pyruvate peroxisomal transport via proton | SLC16A1; | −0.03 |
| symport | SLC16A7 | |
| (S)-Lactate: NAD+ oxidoreductase Glycolysis/ | LDHA; LDHB; | −0.03 |
| Gluconeogenesis/Pyruvate metabolism | LDHC | |
| EC: 1.1.1.27 | | |
| (S)-Lactate: NAD+ oxidoreductase Glycolysis/ | LDHA; LDHB; | −0.03 |
| Gluconeogenesis/Pyruvate metabolism | LDHC | |
| EC: 1.1.1.27 | | |
| sn-Glycerol-3-phosphate: NAD+ 2-oxidoreductase | GPD1 | −0.03 |
| Glycerophospholipid metabolism EC: 1.1.1.8 | | |
| sn-Glycerol-3-phosphate: NAD+ 2-oxidoreductase | GPD1 | −0.03 |
| Glycerophospholipid metabolism EC: 1.1.1.8 | | |
| Free diffusion | | −0.03 |
| Free diffusion | | −0.03 |
| Free diffusion | | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Acetoacetate mitochondrial transport via H+ | SLC16A1 | −0.03 |
| symport | | |
| Acetoacetate mitochondrial transport via H+ | SLC16A1 | −0.03 |
| symport | | |
| release of biotin across the basolatral membrane | | −0.03 |
| Demand for biotin | | −0.03 |
| Biotin exchange | | −0.03 |
| H transporter, peroxisome | | −0.03 |
| Lactate transport | | −0.03 |
| pyruvate mitochondrial transport via proton | SLC16A1 | −0.03 |
| symport | | |
| pyruvate mitochondrial transport via proton | SLC16A1 | −0.03 |
| symport | | |
| Active transport | | −0.03 |
| Active transport | | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| Major Facilitator(MFS) TCDB: 2.A.1.13.1 | SLC16A1 | −0.03 |
| nucleoside-diphosphatase (UDP), extracellular | CANT1; | −0.03 |
| | ENTPD1; | |
| | ENTPD3; | |
| | ENTPD8 | |
| nucleoside-diphosphatase (UTP), extracellular | CANT1; | −0.03 |
| | ENTPD1; | |
| | ENTPD3; | |
| | ENTPD8 | |
| Beta oxidation of long chain fatty acid | ACAA1B; ACOX1; | −0.03 |
| | EHHADH; | |
| | HSD17B4 | |
| N-[(R)-4-Phosphopantothenoyl]-L-cysteine | PPCDC | −0.03 |
| carboxy-lyase Pantothenate and CoA | | |
| biosynthesis EC: 4.1.1.36 | | |
| ATP: pantothenate 4-phosphotransferase | PANK1; PANK2; | −0.03 |
| Pantothenate and CoA biosynthesis EC: 2.7.1.33 | PANK3; PANK4 | |
| Postulated transport reaction | | −0.03 |
| inorganic diphosphatase, endoplasmic reticulum | G6PC; G6PC2; | −0.03 |
| | G6PC3 | |
| phosphatidylinositol-3,4,5-trisphosphate 5- | INPP5A; | −0.03 |
| phosphatase | INPP5B; | |
| | INPP5D; | |
| | INPP5E; INPP5J; | |
| | INPPL1; SYNJ1; | |
| | SYNJ2 | |
| phosphatidylinositol 3,4-bisphosphate 5-kinase | PIP5K1A; | −0.03 |
| | PIP5K1B; | |
| | PIP5K1C | |
| cholesterol monooxygenase | | −0.03 |
| 1-alkyl 2-acylglycerol 3-phosphocholine | | −0.03 |
| transport | | |
| 1-alkyl 2-acteylglycerol 3-phosphocholine | | −0.03 |
| transport | | |
| Y+LAT2 Utilized transport | SLC7A6 | −0.03 |
| Y+LAT2 Utilized transport | SLC7A6 | −0.03 |
| Y+LAT2 Utilized transport | SLC7A6 | −0.03 |
| Y+LAT2 Utilized transport | SLC7A6 | −0.03 |
| Y+LAT2 Utilized transport | SLC7A6 | −0.03 |
| Y+LAT2 Utilized transport | SLC7A6 | −0.03 |
| citrulline sink | | −0.03 |
| Xanthine: NAD+ oxidoreductase Purine | XDH | −0.03 |
| metabolism EC: 1.17.1.4 | | |
| Transport of L-Histidine by y+ transporter | SLC7A1 | −0.03 |
| exchange reaction for L-serine | | −0.03 |
| Arachidonate 5-lipoxygenase | ALOX5 | −0.03 |
| nC20: 4 exchange | | −0.03 |
| sink reaction for 5(S)-HPETE(1-) | | −0.03 |
| RE1342 | | −0.03 |
| Vesicular transport | | −0.03 |
| acn13acngalgbside hs transport | | −0.03 |
| acn13acngalgbside hs intracellular transport | | −0.03 |
| acn23acngalgbside transport | | −0.03 |
| acn23acngalgbside intracellular transport | | −0.03 |
| blood group intracellular transport | | −0.03 |
| blood group intracellular transport | | −0.03 |
| acnacngalgbside hs transport | | −0.03 |
| acnacngalgbside hs intracellular transport | | −0.03 |
| Beta-1,3-galactosyltransferase 4 | B3GALT4 | −0.03 |
| UDP-GlcNAc: betaGal beta-1,3-N- | B3GNT3 | −0.03 |
| acetylglucosaminyltransferase 3, Golgi apparatus | | |
| sialyl (1,3) sialyl (2,6) galactosylgloboside | | −0.03 |
| (homo sapiens)exchange | | |
| sialyl (2,3) sialyl (2,6) galactosylgloboside | | −0.03 |
| (homo sapiens)exchange | | |
| 3′,8′-LD1 exchange | | −0.03 |
| disialyl galactosylgloboside (homo sapiens) | | −0.03 |
| exchange | | |
| GD1beta (homo sapiens) exchange | | −0.03 |
| GD1c (homo sapiens) exchange | | −0.03 |
| Beta-1,4 N-acetylgalactosaminyltransferase | B4GALNT1 | −0.03 |
| gd1b2 hs transport | | −0.03 |
| gd1b2 hs intracellular transport | | −0.03 |
| gd1c hs transport | | −0.03 |
| gd1c hs intracellular transport | | −0.03 |
| Beta-galactoside alpha-2,3-sialyltransferase, | ST3GAL2 | −0.03 |
| Golgi apparatus | | |
| CMP-N-acetylneuraminate-beta- | ST3GAL2 | −0.03 |
| galactosamide-alpha-2,3-sialyltransferase, | | |
| Golgi apparatus | | |
| Alpha-N-acetylgalactosaminide alpha-2,6- | ST6GALNAC2 | −0.03 |
| sialyltransferase 2 | | |
| Alpha-N-acetylgalactosaminide alpha-2,6- | ST6GALNAC2 | −0.03 |
| sialyltransferase 2 | | |
| Alpha-N-acetylgalactosaminide alpha-2,6- | ST6GALNAC2 | −0.03 |
| sialyltransferase 2 | | |
| Alpha-N-acetylgalactosaminide alpha-2,6- | ST6GALNAC2 | −0.03 |
| sialyltransferase 2 | | |
| Beta-galactoside alpha-2,3-sialyltransferase | ST8SIA1 | −0.03 |
| Beta-galactoside alpha-2,3-sialyltransferase | ST8SIA5 | −0.03 |
| Alpha-2,8-sialyltransferase 8E | ST8SIA5 | −0.03 |
| alpha-mannosidase, lysosomal | MAN2B1 | −0.03 |
| beta-N-acetylhexosaminidase, lysosomal | HEXA; HEXB | −0.03 |
| Asn-X-Ser/Thr transport (from ER to | | −0.03 |
| lysosome) | | |
| beta-mannosidase, lysosomal | MANBA | −0.03 |
| GDPmannose: chitobiosyldiphosphodolichol | ALG1 | −0.03 |
| beta-D-mannosyltransferase (liver) | | |
| GDPmannose: chitobiosyldiphosphodolichol | ALG1 | −0.03 |
| beta-D-mannosyltransferase (uterus) | | |
| Dolichyl-diphosphooligosaccharide: protein- | DAD1; DDOST; | −0.03 |
| L-asparagine oligopolysaccharidotransferase | RPN1; RPN2 | |
| (liver) | | |
| Dolichyl-diphosphooligosaccharide: protein- | DAD1; DDOST; | −0.03 |
| L-asparagine oligopolysaccharidotransferase | RPN1; RPN2 | |
| (uterus) | | |
| Dolichyl-diphosphate phosphohydrolase, | DOLPP1 | −0.03 |
| human (liver) | | |
| Dolichyl-diphosphate phosphohydrolase, | DOLPP1 | −0.03 |
| human (uterus) | | |
| dolichol-phosphate mannose flippase (liver) | | −0.03 |
| dolichol-phosphate mannose flippase | | −0.03 |
| (uterus) | | |
| dolichol phosphate flippase (liver) | | −0.03 |
| dolichol phosphate flippase (uterus) | | −0.03 |
| dolichyl-phosphate-glucose-glycolipid alpha- | ALG6 | −0.03 |
| glucosyltransferase (liver) | | |
| dolichyl-phosphate-glucose-glycolipid alpha- | ALG6 | −0.03 |
| glucosyltransferase (uterus) | | |
| dolichyl-phosphate-glucose-glycolipid alpha- | ALG8 | −0.03 |
| glucosyltransferase (liver) | | |
| dolichyl-phosphate-glucose-glycolipid alpha- | ALG8 | −0.03 |
| glucosyltransferase (uterus) | | |
| Dolichol-phosphate phosphohydrolase, | | −0.03 |
| human (liver) | | |
| Dolichol-phosphate phosphohydrolase, | | −0.03 |
| human (uterus) | | |
| Dolichyl-phosphate D-mannosyltransferase | DPM2; DPM3; | −0.03 |
| (liver) | GM20716 | |
| Dolichyl-phosphate D-mannosyltransferase | DPM2; DPM3; | −0.03 |
| (uterus) | GM20716 | |
| dolichyl-phosphate-mannose-glycolipid | ALG3 | −0.03 |
| alpha-mannosyltransferase (liver) | | |
| dolichyl-phosphate-mannose-glycolipid | ALG3 | −0.03 |
| alpha-mannosyltransferase (uterus) | | |
| dolichyl-phosphate-mannose-glycolipid | | −0.03 |
| alpha-mannosyltransferase (liver) | | |
| dolichyl-phosphate-mannose-glycolipid | | −0.03 |
| alpha-mannosyltransferase (uterus) | | |
| dolichyl-phosphate-mannose-glycolipid | ALG12 | −0.03 |
| alpha-mannosyltransferase (liver) | | |
| dolichyl-phosphate-mannose-glycolipid | ALG12 | −0.03 |
| alpha-mannosyltransferase (uterus) | | |
| endo-beta-N-acetylglucosaminidase, | | −0.03 |
| lysosomal | | |
| Glycolipid 1,2-alpha-D-mannosyltransferase | | −0.03 |
| (liver) | | |
| Glycolipid 1,2-alpha-D-mannosyltransferase | | −0.03 |
| (uterus) | | |
| Glycolipid 1,2-alpha-D-mannosyltransferase | | −0.03 |
| (liver) | | |
| Glycolipid 1,2-alpha-D-mannosyltransferase | | −0.03 |
| (uterus) | | |
| Glycolipid 1,3-alpha-D-mannosyltransferase | ALG2 | −0.03 |
| (liver) | | |
| Glycolipid 1,3-alpha-D-mannosyltransferase | ALG2 | −0.03 |
| (uterus) | | |
| Glycolipid 1,6-alpha-D-mannosyltransferase | | −0.03 |
| (liver) | | |
| Glycolipid 1,6-alpha-D-mannosyltransferase | | −0.03 |
| (uterus) | | |
| glycosylasparaginase, lysosomal | AGA | −0.03 |
| UDP-GlcNAc: dolichol-phosphate GlcNAc | DPAGT1 | −0.03 |
| phosphotransferase (liver) | | |
| UDP-GlcNAc: dolichol-phosphate GlcNAc | DPAGT1 | −0.03 |
| phosphotransferase (uterus) | | |
| UDP-GlcNAc: N-acetyl-D-glucosaminyl | | −0.03 |
| diphosphodolichol N-acetyl-D- | | |
| glucosaminyltransferase (liver) | | |
| UDP-GlcNAc: N-acetyl-D-glucosaminyl | | −0.03 |
| diphosphodolichol N-acetyl-D- | | |
| glucosaminyltransferase (uterus) | | |
| mannosyl-oligosaccharide 1,3-1,6-alpha- | MAN2A1; | −0.03 |
| mannosidase | MAN2A2 | |
| alpha-1,3-mannosyl-glycoprotein 2-beta-N- | MGAT1 | −0.03 |
| acetylglucosaminyltransferase | | |
| beta-1,4-mannosyl-glycoprotein 4-beta-N- | MGAT3 | −0.03 |
| acetylglucosaminyltransferase | | |
| alpha-1,6-mannosyl-glycoprotein 2-beta-N- | MGAT2 | −0.03 |
| acetylglucosaminyltransferase | | |
| m4mpdol flippase | | −0.03 |
| m4mpdol flippase | | −0.03 |
| m7masnB transport from endoplasmic | | −0.03 |
| reticulum to Golgi apparatus | | |
| m8masn transport from ER to Golgi | | −0.03 |
| apparatus | | |
| mannose efflux from endoplasmic reticulum | | −0.03 |
| mannose efflux from Golgi apparatus | | −0.03 |
| mannose efflux from lysosome | | −0.03 |
| mannosyl-oligosaccharide glucosidase, | MOGS | −0.03 |
| endoplasmic reticulum | | |
| mannosyl-oligosaccharide glucosidase, | | −0.03 |
| endoplasmic reticulum | | |
| mannosyl-oligosaccharide glucosidase, | | −0.03 |
| endoplasmic reticulum | | |
| mannosyl-oligosaccharide 1,2-alpha- | MAN1A; MAN1A2; | −0.03 |
| mannosidase, Golgi apparatus | MAN1C1 | |
| mannosyl-oligosaccharide 1,2-alpha- | MAN1A; MAN1A2; | −0.03 |
| mannosidase, Golgi apparatus | MAN1C1 | |
| mannosyl-oligosaccharide 1,2-alpha- | MAN1A; MAN1A2; | −0.03 |
| mannosidase, Golgi apparatus | MAN1C1 | |
| mannosyl-oligosaccharide 1,2-alpha- | MAN1C1 | −0.03 |
| mannosidase, Golgi apparatus | | |
| mannosyl-oligosaccharide 1,2-alpha- | MAN1A; MAN1A2; | −0.03 |
| mannosidase, Golgi apparatus | MAN1C1 | |
| mannosyl-oligosaccharide 1,2-alpha- | MAN1A; MAN1A2; | −0.03 |
| mannosidase, Golgi apparatus | MAN1C1 | |
| mannosyl-oligosaccharide 1,2-alpha- | MAN1A; MAN1A2; | −0.03 |
| mannosidase, Golgi apparatus | MAN1C1 | |
| mannosyl-oligosaccharide 1,2-alpha- | MAN1A; MAN1A2; | −0.03 |
| mannosidase, Golgi apparatus | MAN1C1 | |
| mannosyl-oligosaccharide 1,2-alpha- | MAN1C1 | −0.03 |
| mannosidase, Golgi apparatus | | |
| mannosyl-oligosaccharide 1,2-alpha- | MAN1A; MAN1A2; | −0.03 |
| mannosidase, Golgi apparatus | MAN1C1 | |
| mannosyl-oligosaccharide 1,2-alpha- | MAN1A; MAN1A2; | −0.03 |
| mannosidase, Golgi apparatus | MAN1C1 | |
| mannosyl-oligosaccharide 1,2-alpha- | MAN1A; MAN1A2; | −0.03 |
| mannosidase, Golgi apparatus | MAN1C1 | |
| mannosyl-oligosaccharide 1,2-alpha- | MAN1A; MAN1A2; | −0.03 |
| mannosidase, Golgi apparatus | MAN1C1 | |
| mannosyl-oligosaccharide 1,2-alpha- | MAN1A; MAN1A2; | −0.03 |
| mannosidase, Golgi apparatus | MAN1C1 | |
| mannosyl-oligosaccharide 1,2-alpha- | MAN1B1 | −0.03 |
| mannosidase, endoplasmic reticulum | | |
| mannosyl-oligosaccharide 1,2-alpha- | MAN1A; MAN1A2; | −0.03 |
| mannosidase, Golgi apparatus | MAN1C1 | |
| n2m2nmasn transport, Golgi to extracellular | | −0.03 |
| n2m2nmasn transport, extracellular to | | −0.03 |
| lysosome | | |
| phosphomannomutase | PGM1; PMM1; | −0.03 |
| | PMM2 | |
| UDP-GlcNAc Golgi transport via CMP antiport | SLC35D2 | −0.03 |
| o2 transport (diffusion) | | −0.03 |
| transport of L-Phenylalanine into the | SLC6A14 | −0.03 |
| intestinal cells by ATB0 transporter | | |
| diacylglycerol ER export | | −0.03 |
| DM dgpi prot hs(r) | | −0.03 |
| glycophosphatidylinositol (GPI) deacylase, | PGAP1 | −0.03 |
| endoplasmic reticulum | | |
| H6′ phosphoethanolaminyl transferase, | PIGF; PIGG | −0.03 |
| endoplasmic reticulum | | |
| H7′ phosphoethanolaminyl transferase, | PIGN | −0.03 |
| endoplasmic reticulum | | |
| H7 phosphoethanolaminyl transferase, | PIGF; PIGG | −0.03 |
| endoplasmic reticulum | | |
| H8 transamidase, endoplasmic reticulum | GPAA1; PIGK; | −0.03 |
| | PIGS; PIGT; PIGU | |
| deoxyguanosine kinase | DGUOK | −0.03 |
| deoyguanosine transport in mitochondria | | −0.03 |
| Facilitated diffusion | | −0.03 |
| L-citrulline exchange | | −0.03 |
| glyoxylate oxidase, peroxisomal | HAO1; HAO2 | −0.03 |
| oxalate transport out of peroxisome | | -0.03 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Ornithine Decarboxylase | ODC1 | −0.04 |
| 6,7-dihydropteridine reduction | | −0.04 |
| deltal-piperideine-2-carboxylate reductase, | | −0.04 |
| perixosomal | | |
| 2-aminoadipate transaminase, irreversible | AADAT | −0.04 |
| L-pipecolate oxidase, peroxisomal | PIPOX | −0.04 |
| L-lysine oxidase, peroxisomal | | −0.04 |
| L-lysine transport, peroxisomal | | −0.04 |
| (irreversible) | | |
| PPD2CSPp | | −0.04 |
| RE1254 | | −0.04 |
| 2,3,4,5-Tetrahydropyridine-2-carboxylate | | −0.04 |
| transport, peroxisomal | | |
| EC: 6.2.1.3 | | −0.04 |
| RE3241 | ELOVL2; ELOVL4; | −0.04 |
| | ELOVL5; ELOVL6 | |
| RE3242 | | −0.04 |
| RE3243 | | −0.04 |
| RE3244 | | −0.04 |
| (5-Glutamyl)-peptide: amino-acid 5- | GGT1; GGT5; | −0.04 |
| glutamyltransferase Arachidonic acid | GGT6; GGT7 | |
| metabolism EC: 2.3.2.2 | | |
| RE2079 | | −0.04 |
| 1,3-Diaminopropane: oxygen oxidoreductase | AOC1; AOC2; | −0.04 |
| (deaminating) | AOC3 | |
| beta-Aminopropion aldehyde: NAD+ | ALDH1A1; | −0.04 |
| oxidoreductase | ALDH1A2; | |
| | ALDH3A2; | |
| | ALDH7A1; | |
| | ALDH9A1 | |
| fatty acid beta oxidation(C10: 2-->C10: 3)m | ACADM | −0.04 |
| fatty acid beta oxidation(C10: 2-->C8: 1)m | ACAA2; ECHS1; | −0.04 |
| | HADH | |
| isomerization(C10: 2)m | ECU | −0.04 |
| fatty acid beta oxidation(C10: 3-->C10: 2)m | DECR1 | −0.04 |
| fatty acid beta oxidation(C12: 3-->C10: 2)m | ACAA2; ECHS1; | −0.04 |
| | HADH | |
| isomerization(C12: 3)m | ECU | −0.04 |
| fatty acid beta oxidation(C14: 3-->C12: 3)m | ACADVL; HADHA; | −0.04 |
| | HADHB | |
| fatty acid beta oxidation(C16: 4-->C14: 3)m | HADHA; HADHB | −0.04 |
| fatty acid beta oxidation(C16: 4-->C16: 5)m | ACADVL | −0.04 |
| isomerization(C16: 4)m | ECU | −0.04 |
| fatty acid beta oxidation (C16: 5-->C16: 4)m | DECR1 | −0.04 |
| fatty acid beta oxidation(C18: 4-->C16: 4)m | ACADVL; HADHA; | −0.04 |
| | HADHB | |
| fatty acid beta oxidation(C6: 1-->C4: 0)m | ACAA2; ECHS1; | −0.04 |
| | HADH | |
| isomerization(C6: 1)m | ECU | −0.04 |
| fatty acid beta oxidation(C8: 1-->C6: 1)m | ACAA2; ACADM; | −0.04 |
| | ECHS1; HADH | |
| Y+LAT2 Utilized transport | SLC7A6 | −0.04 |
| fatty acid intracellular transport | | −0.04 |
| methylmalonyl-CoA epimerase/racemase | MCEE | −0.04 |
| malonate-semialdehyde dehydrogenase | | −0.04 |
| (acetylating), mitochondrial | | |
| (S)-Methylmalonyl-CoA hydrolase | | −0.04 |
| Propanoate metabolism EC: 3.1.2.17 | | |
| (S)-Methylmalonate semialdehyde: NAD+ | ALDH1B1; | −0.04 |
| oxidoreductase Valine, leucine and isoleucine | ALDH2; | |
| degradation EC: 1.2.1.3 | ALDH3A2; | |
| | ALDH7A1; | |
| | ALDH9A1 | |
| fatty acid beta oxidation(C18: 1-->C16: 1)m | ACADVL; HADHA; | −0.04 |
| | HADHB | |
| transport of 11-octadecenoyl carnitine into | CPT1A; CPT1B; | −0.04 |
| the mitochondrial matrix | CPT1C | |
| transport of 11-octadecenoyl carnitine into | SLC25A20 | −0.04 |
| the mitochondrial matrix | | |
| transport of 11-octadecenoyl carnitine into | CPT2 | −0.04 |
| the mitochondrial matrix | | |
| uptake of vaccenic acid by cells | SLC27A1; | −0.04 |
| | SLC27A2; | |
| | SLC27A3; | |
| | SLC27A4; | |
| | SLC27A5; | |
| | SLC27A6 | |
| acetyl-CoA acyltransferase (tetradecanoyl- | ACAA1B | −0.04 |
| CoA), peroxisomal | | |
| fatty acid beta oxidation(C10-->C8)x | ACAA1B; ACOX1; | −0.04 |
| | EHHADH; | |
| | HSD17B4 | |
| fatty acid beta oxidation(C12-->C10)x | ACAA1B; ACOX1; | −0.04 |
| | EHHADH; | |
| | HSD17B4 | |
| fatty acid beta oxidation(C14-->C12)x | ACAA1B; ACOX1; | −0.04 |
| | EHHADH; | |
| | HSD17B4 | |
| hdd2coa intracellular transport | | −0.04 |
| fatty acid intracellular transport | | −0.04 |
| (S)-3-Hydroxyhexadecanoyl-CoA: NAD+ | EHHADH; HADH; | −0.04 |
| oxidoreductase Fatty acid elongation in | HADHA | |
| mitochondria/Fatty acid metabolism | | |
| EC: 1.1.1.35 EC: 1.1.1.211 | | |
| (S)-3-Hydroxyhexadecanoyl-CoA hydro-lyase | ECHS1; EHHADH; | −0.04 |
| Fatty acid elongation in mitochondria/Fatty | HADHA | |
| acid metabolism EC: 4.2.1.17 | | |
| hydroxyacylglutathione hydrolase | HAGH; HAGHL | −0.04 |
| D-lactate dehydrogenase | LDHD | −0.04 |
| acetone transport via proton symport | SLC16A1; | −0.04 |
| | SLC16A3; | |
| | SLC16A7 | |
| acetone mitochondrial transport via proton | SLC16A1 | −0.04 |
| symport | | |
| Acetoacetate decarboxylation (irreversible), | | −0.04 |
| mitochondrial | | |
| Acetone exchange | | −0.04 |
| N-acetylneuraminate nuclear import | | −0.04 |
| CMP-Sia nuclear export | | −0.04 |
| CMP sialic acid synthase | CMAS | −0.04 |
| CMP sialic acid synthase, nuclear | CMAS | −0.04 |
| CTP diffusion in nucleus | | −0.04 |
| N-acetylneuraminate, ferrocytochrome- | | −0.04 |
| b5: oxygen oxidoreductase (N-acetyl- | | |
| hydroxylating) Aminosugars metabolism | | |
| EC: 1.14.18.2 | | |
| CTP: N-acylneuraminate cytidylyltransferase | CMAS | −0.04 |
| Aminosugars metabolism EC: 2.7.7.43 | | |
| Exchange of Flavin adenine dinucleotide | | −0.04 |
| oxidized | | |
| exchange reaction for FMN | | −0.04 |
| Lactose exchange | | −0.04 |
| 3-Sulfoalanine carboxy-lyase | CSAD; GAD1; | −0.04 |
| | GAD2 | |
| 5′-nucleotidase (dGMP) | NT5C; NT5C1A; | −0.04 |
| | NT5C1B; NT5C2; | |
| | NT5C3; NT5E | |
| ATP: deoxyguanosine 5-phosphotransferase | DGUOK | −0.04 |
| Purine metabolism EC: 2.7.1.113 | | |
| Fructose-2,6-bisphosphate 2-phosphatase | FBP1; FBP2; | −0.04 |
| | PFKFB1; PFKFB2; | |
| | PFKFB3; PFKFB4 | |
| 6-phosphofructo-2-kinase | PFKFB1; PFKFB2; | −0.04 |
| | PFKFB3; PFKFB4 | |
| Formate exchange | | −0.04 |
| exchange reaction for FMN | | −0.04 |
| transport of sebacoyl carnitine into cytosol | SLC25A20 | −0.04 |
| excretion of C12DC | | −0.04 |
| exchange reaction for dodecanedioyl | | −0.04 |
| carnitine | | |
| fatty acid beta oxidation(C12DC-->C10DC)x | ACAA1B; ACOX1; | −0.04 |
| | EHHADH; | |
| | HSD17B4 | |
| transport of dodecanedioyl carnitine into | SLC25A20 | −0.04 |
| cytosol | | |
| production of dodecanedioyl carnitine | CROT | −0.04 |
| fatty acid beta oxidation(C14DC-->C12DC)x | ACAA1B; ACOX1; | −0.04 |
| | EHHADH; | |
| | HSD17B4 | |
| fatty acid beta oxidation(C16DC-->C14DC)x | ACAA1B; ACOX1; | −0.04 |
| | EHHADH; | |
| | HSD17B4 | |
| transport of hexadecanedioyl coa | DBI | −0.04 |
| production of sebacoyl carnitine | CROT | −0.04 |
| 5-hydroxy-L-tryptophan secretion via | SLC18A1; | −0.04 |
| secretory vesicle (ATP driven) | SLC18A2 | |
| 5-Hydroxy-L-tryptophan exchange | | −0.04 |
| L-Tryptophan exchange | | −0.04 |
| L-Tryptophan, tetrahydrobiopterin: oxygen | TPH1; TPH2 | −0.04 |
| oxidoreductase (5-hydroxylating) | | |
| Tryptophan metabolism EC: 1.14.16.4 | | |
| fatty acid beta oxidation(C16: 3-->C14: 3)m | ACADVL; HADHA; | −0.04 |
| | HADHB | |
| fatty acid beta oxidation(C18: 3-->C16: 3)m | ACADVL; HADHA; | −0.04 |
| | HADHB | |
| 3,4-Dihydroxyphenylacetaldehyde: NADP+ | ALDH3A1; | −0.04 |
| oxidoreductase | ALDH3B1; | |
| | ALDH3B3 | |
| 3,4-Dihydroxyphenylacetaldehyde: NAD+ | ALDH1A3; | −0.04 |
| oxidoreductase | ALDH3A1; | |
| | ALDH3B1; | |
| | ALDH3B3 | |
| 3,4-Dihydroxymandelaldehyde: NADP+ | ALDH3B1; | −0.04 |
| oxidoreductase | ALDH3B3 | |
| 3,4-Dihydroxymandelaldehyde: NAD+ | ALDH1A3; | −0.04 |
| oxidoreductase | ALDH3A1; | |
| | ALDH3B1 | |
| 3-Methoxy-4- | ALDH1A3; | −0.04 |
| hydroxyphenylglycolaldehyde: NADP+ | ALDH3A1; | |
| oxidoreductase | ALDH3B1; | |
| | ALDH3B3 | |
| 3-Methoxy-4- | ALDH1A3; | −0.04 |
| hydroxyphenylglycolaldehyde: NAD+ | ALDH3A1; | |
| oxidoreductase | ALDH3B1; | |
| | ALDH3B3 | |
| dihydrouracil dehydrogenase (NADP) | DPYD | −0.04 |
| 5,6-Dihydrouracil: NAD+ oxidoreductase | | −0.04 |
| Pyrimidine metabolism EC: 1.3.1.1 | | |
| CMP-N-acetylneuraminate, ferrocytochrome- | | −0.04 |
| b5: oxygen oxidoreductase (N-acetyl- | | |
| hydroxylating) Aminosugars metabolism | | |
| EC: 1.14.18.2 | | |
| cytidine monophospho-N-acetylneuraminic | | −0.04 |
| acid hydroxylase EC: 1.14.18.2 | | |
| Phenylacetaldehyde: NAD+ oxidoreductase | ALDH1A3; | −0.04 |
| Phenylalanine metabolism/Styrene | ALDH3A1; | |
| degradation EC: 1.2.1.5 EC: 1.2.1.39 | ALDH3B1; | |
| | ALDH3B3 | |
| Aldehyde: NADP+ oxidoreductase | ALDH1A3; | −0.04 |
| Phenylalanine metabolism EC: 1.2.1.5 | ALDH3A1; | |
| | ALDH3B1; | |
| | ALDH3B3 | |
| RE1342 | | −0.04 |
| D-sorbitol reductase | AKR1A1; AKR1B3; | −0.04 |
| | AKR7A5 | |
| DM n5m2masn(g) | | −0.04 |
| alpha-l,3-mannosyl-glycoprotein 4-beta-N- | MGAT4A; | −0.04 |
| acetylglucosaminyltransferase | MGAT4B | |
| alpha-l,6-mannosyl-glycoprotein 4-beta-N- | | −0.04 |
| acetylglucosaminyltransferase | | |
| alpha-l,6-mannosyl-glycoprotein 6-beta-N- | MGAT5 | −0.04 |
| acetylglucosaminyltransferase | | |
| Phosphopantetheine adenylyltransferase (EC | COASY | −0.04 |
| 2.7.7.3) | | |
| Dephospho-CoA nucleotidohydrolase | ENPP1; ENPP3 | −0.04 |
| Pantothenate and CoA biosynthesis | | |
| EC: 3.6.1.9 | | |
| glycine hydroxymethyltransferase, reversible | SHMT1 | −0.04 |
| 10-Formyltetrahydrofolate mitochondrial | | −0.04 |
| transport via diffusion | | |
| glycine hydroxymethyltransferase, | SHMT1; SHMT2 | −0.04 |
| reversible, mitochondrial | | |
| glycine passive transport to mitochondria | | −0.04 |
| methenyltetrahydrofolate cyclohydrolase | MTHFD1; | −0.04 |
| | MTHFD2L | |
| methenyltetrahydrifikate cyclohydrolase, | MTHFD1L; | −0.04 |
| mitochondrial | MTHFD2 | |
| Transport reaction | | −0.04 |
| L-leucine transport via diffusion | SLC43A1; | −0.04 |
| (extracellular to cytosol) | SLC43A2 | |
| Dopamine 3-O-sulfate transport (diffusion) | | −0.04 |
| Dopamine Sulfotransferase | SULT1A1 | −0.04 |
| Dopamine 3-O-sulfate exchange | | −0.04 |
| alcohol dehydrogenase (methanol) | ADH1; ADH4; | −0.04 |
| | ADH5; ADH6A; | |
| | ADH7; ADHFE1; | |
| | ZADH2 | |
| Na+/K+ exchanging ATPase | ATP1A1; ATP1A2; | −0.04 |
| | ATP1A3; ATP1A4; | |
| | ATP1B1; ATP1B2; | |
| | ATP1B3; ATP1B4 | |
| Beta oxidation of long chain fatty acid | ACADM; ACADS | −0.04 |
| Beta oxidation of long chain fatty acid | ACADM; ACADS | −0.04 |
| transport of Farnesyl diphosphate into the | | −0.04 |
| endoplasmic reticulum | | |
| Phosphoenolpyruvate carboxykinase (GTP) | PCK1; PCK2 | −0.04 |
| 5′-nucleotidase (UMP) | NT5C3 | −0.04 |
| dUTP: uridine 5-phosphotransferase | UCK1; UCK2; | −0.04 |
| Pyrimidine metabolism EC: 2.7.1.48 | UCKL1 | |
| fatty acid beta oxidation(C10: 2-->C10: 3)x | ACOX1 | −0.04 |
| fatty acid beta oxidation(C10: 2-->C8: 1)x | ACAA1B; | −0.04 |
| | EHHADH; | |
| | HSD17B4 | |
| isomerization(C10: 2)x | ECI3; EHHADH | −0.04 |
| fatty acid beta oxidation(C10: 3-->C10: 2)x | DECR2 | −0.04 |
| fatty acid beta oxidation(C12: 3-->C10: 2)x | ACAA1B; | −0.04 |
| | EHHADH; | |
| | HSD17B4 | |
| isomerization(C12: 3)x | ECI3; EHHADH | −0.04 |
| fatty acid beta oxidation(C14: 3-->C12: 3)x | ACAA1B; ACOX1; | −0.04 |
| | EHHADH; | |
| | HSD17B4 | |
| fatty acid beta oxidation(C16: 4-->C14: 3)x | ACAA1B; | −0.04 |
| | EHHADH; | |
| | HSD17B4 | |
| fatty acid beta oxidation(C16: 4-->C16: 5)x | ACOX1 | −0.04 |
| isomerization(C16: 4)x | ECI3; EHHADH | −0.04 |
| fatty acid beta oxidation(C16: 5-->C16: 4)x | DECR2 | −0.04 |
| fatty acid beta oxidation(C18: 4->C16: 4)x | ACAA1B; ACOX1; | −0.04 |
| | EHHADH; | |
| | HSD17B4 | |
| fatty acid beta oxidation(C6: 1-->C4: 0)x | ACAA1B; | −0.04 |
| | EHHADH; | |
| | HSD17B4 | |
| isomerization(C6: 1)x | ECI3; EHHADH | −0.04 |
| fatty acid beta oxidation(C8: 1-->C6: 1)x | ACAA1B; ACOX1; | −0.04 |
| | EHHADH; | |
| | HSD17B4 | |
| dGTP diffusion in nucleus | | −0.04 |
| DM dgtp(m) | | −0.04 |
| DM dgtp(n) | | −0.04 |
| RE3347 | | −0.04 |
| 24-dehydrocholesterol reductase | DHCR24 | −0.04 |
| [Precursor] | | |
| 24-dehydrocholesterol reductase | CYP7A1; DHCR24 | −0.04 |
| [Precursor] | | |
| FADH2 transporter, endoplasmic reticulum | | −0.04 |
| FAD transporter, endoplasmic reticulum | | −0.04 |
| lanosterol D24-reductase Biosynthesis of | DHCR24 | −0.04 |
| steroids EC: 1.3.1.72 | | |
| Free diffusion | | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| transport of 3aib_D into mitochondria | | −0.04 |
| beta-ureidopropionase (D-3-amino- | UPB1 | −0.04 |
| isobutanoate forming) | | |
| dihydropyrimidinase (dihydrothymine) | DPYS | −0.04 |
| dihydrothymin dehydrogenase (NADP) | DPYD | −0.04 |
| gamma-linolenic acid exchange | | −0.04 |
| fatty-acid--CoA ligase | ACSBG2; ACSL1; | −0.04 |
| | ACSL3; ACSL4; | |
| | ACSL5; ACSL6 | |
| fatty-acid--CoA ligase | ACSBG2; ACSL1; | −0.04 |
| | ACSL3; ACSL4 | |
| bile acid Coenzyme A: amino acid N- | BAAT | −0.04 |
| acyltransferase | | |
| sodium transport via diffusion (perioxisome) | | −0.04 |
| sodium transport via diffusion (perioxisome) | | −0.04 |
| Choloyl-CoA: glycine N-choloyltransferase | BAAT | −0.04 |
| Bile acid biosynthesis/Taurine and | | |
| hypotaurine metabolism EC: 2.3.1.65 | | |
| Neurotransmitter: Sodium Symporter (NSS) | SLC6A6 | −0.04 |
| TCDB: 2.A.22.3.3 | | |
| Postulated transport reaction | | −0.04 |
| Postulated transport reaction | | −0.04 |
| Postulated transport reaction | | −0.04 |
| RE1845 | BAAT | −0.04 |
| RE1845 | BAAT | −0.04 |
| RE1845 | BAAT | −0.04 |
| RE1845 | BAAT | −0.04 |
| taurine transport (sodium symport) (cytosol | SLC6A6 | −0.04 |
| to peroxisome) | | |
| taurine transport (sodium symport) (cytosol | SLC6A6 | −0.04 |
| to peroxisome) | | |
| bile acid intracellular transport | | −0.04 |
| bile acid intracellular transport | | −0.04 |
| bile acid intracellular transport | | −0.04 |
| Thiamin monophosphate exchange | | −0.04 |
| Thiamine monophosphate transport in via | SLC19A1 | −0.04 |
| anion antiport | | |
| deoxyguanylate kinase (dGMP: dATP) | | −0.04 |
| (mitochondrial) | | |
| Ribitol exchange | | −0.04 |
| Ribitol: NAD+ 2-oxidoreductase Pentose and | | −0.04 |
| glucuronate interconversions EC: 1.1.1.56 | | |
| ribitol transport via passive diffusion | | −0.04 |
| methylcrotonoyl-CoA carboxylase, | MCCC1; MCCC2 | −0.04 |
| mitochondrial | | |
| methylglutaconyl-CoA hydratase | AUH | −0.04 |
| (reversible), mitochondrial | | |
| (R)-3-Hydroxybutanoate: NAD+ | BDH1 | −0.04 |
| oxidoreductase | | |
| (R)-3-Hydroxybutanoate mitochondrial | | −0.04 |
| transport via H+ symport | | |
| (R)-3-Hydroxybutanoate transport via H+ | | −0.04 |
| symport | | |
| dNAD transport, nuclear trhough pore | | −0.04 |
| NICRNT transport, nuclear trhough pore | | −0.04 |
| nicotinate-nucleotide adenylyltransferase | NMNAT1 | −0.04 |
| glycerate kinase | | −0.04 |
| ATP: (R)-glycerate 3-phosphotransferase | GLYCTK | −0.04 |
| Glycine, serine and threonine metabolism | | |
| EC: 2.7.1.31 | | |
| Transport of glucose into the portal blood | SLC2A2 | −0.04 |
| glucose transport via membrane vesicle | | −0.04 |
| glucose transport, endoplasmic reticulum | | −0.04 |
| fatty acid beta oxidation(C10: 1-->C10: 2)x | ACOX1 | −0.04 |
| fatty acid beta oxidation(C10: 1-->C8: 0)x | ACAA1B; | −0.04 |
| | EHHADH; | |
| | HSD17B4 | |
| isomerization(C10: 1)x | ECI3; EHHADH | −0.04 |
| fatty acid beta oxidation(C10: 2-->C10: 1)x | DECR2 | −0.04 |
| isomerization(C12: 2)x | ECI3; EHHADH | −0.04 |
| fatty acid beta oxidation(C14: 2-->C12: 2)x | ACAA1B; ACOX1; | −0.04 |
| | EHHADH; | |
| | HSD17B4 | |
| fatty acid beta oxidation(C16: 3-->C14: 2)x | ACAA1B; | −0.04 |
| | EHHADH; | |
| | HSD17B4 | |
| fatty acid beta oxidation(C16: 3-->C16: 4)x | ACOX1 | −0.04 |
| isomerization(C16: 3)x | ECI3; EHHADH | −0.04 |
| fatty acid beta oxidation(C16: 4-->C16: 3)x | DECR2 | −0.04 |
| fatty acid beta oxidation(C18: 4-->C16: 3)x | ACAA1B; | −0.04 |
| | EHHADH; | |
| | HSD17B4 | |
| isomerization(C18: 4)x | ECI3; EHHADH | −0.04 |
| fatty acid beta oxidation(C20: 4-->C20: 5)x | ACOX1 | −0.04 |
| fatty acid beta oxidation(C20: 5-->C18: 4)x | ACAA1B; | −0.04 |
| | EHHADH; | |
| | HSD17B4 | |
| fatty acid beta oxidation(C22: 5-->C20: 4)x | ACAA1B; | −0.04 |
| | EHHADH; | |
| | HSD17B4 | |
| fatty acid beta oxidation(C22: 5-->C22: 6)x | ACOX1 | −0.04 |
| isomerization(C22: 5)x | ECI3; EHHADH | −0.04 |
| fatty acid beta oxidation(C22: 6-->C22: 5)x | DECR2 | −0.04 |
| fatty acid beta oxidation(C12: 2-->C10: 1)x | ACAA1B; | −0.04 |
| | EHHADH; | |
| | HSD17B4 | |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Transport of 5-oxoprolinate | | −0.04 |
| Exchange of 5-oxoprolinate | | −0.04 |
| alcohol dehydrogenase (ethanol, NADP), | AKR1A1 | −0.04 |
| forward reaction | | |
| Prostaglandin F2alpha exchange | | −0.04 |
| ethanol reversible transport | | −0.04 |
| Ethanol exchange | | −0.04 |
| acetoacetate intracellular transport | | −0.04 |
| unknown mechanism | | |
| hydroxymethylglutaryl-CoA lyase | HMGCL | −0.04 |
| RE1901 | | −0.04 |
| transport of Methionine by y+LAT1 or | SLC3A2; SLC7A6; | −0.04 |
| y+LAT2 with co-transporter of h in small | SLC7A7 | |
| intestine and kidney | | |
| acetyl-CoA C-acetyltransferase (octanoyl- | ACAA1B | −0.04 |
| CoA), peroxisomal | | |
| acetyl-CoA C-acyltransferase (decanoyl-CoA), | ACAA1B | −0.04 |
| peroxisomal | | |
| acetyl-CoA C-acetyltransferase (dodecanoyl), | ACAA1B | −0.04 |
| peroxisomal | | |
| fatty acid beta xoidation(C12: 1-->C10)x | ACAA1B; | −0.04 |
| | EHHADH; | |
| | HSD17B4 | |
| (S)-3-Hydroxydodecanoyl-CoA hydro-lyase | ECHS1; EHHADH; | −0.04 |
| Fatty acid elongation in mitochondria/Fatty | HADHA | |
| acid metabolism EC: 4.2.1.17 | | |
| (S)-3-Hydroxytetradecanoyl-CoA: NAD+ | EHHADH; HADH; | −0.04 |
| oxidoreductase Fatty acid elongation in | HADHA | |
| mitochondria/Fatty acid metabolism | | |
| EC: 1.1.1.35 EC: 1.1.1.211 | | |
| (S)-3-Hydroxytetradecanoyl-CoA hydro- | ECHS1; EHHADH; | −0.04 |
| lyase Fatty acid elongation in mitochondria/ | HADHA | |
| Fatty acid metabolism EC: 4.2.1.17 | | |
| (S)-3-Hydroxydodecanoyl-CoA: NAD+ | EHHADH; HADH; | −0.04 |
| oxidoreductase Fatty acid elongation in | HADHA | |
| mitochondria/Fatty acid metabolism | | |
| EC: 1.1.1.211 EC: 1.1.1.35 | | |
| (S)-Hydroxydecanoyl-CoA: NAD+ | EHHADH; HADH; | −0.04 |
| oxidoreductase Fatty acid elongation in | HADHA | |
| mitochondria/Fatty acid metabolism | | |
| EC: 1.1.1.211 EC: 1.1.1.35 | | |
| (S)-Hydroxydecanoyl-CoA hydro-lyase Fatty | ECHS1; EHHADH; | −0.04 |
| acid elongation in mitochondria/Fatty acid | HADHA | |
| metabolism EC: 4.2.1.17 | | |
| EC: 1.3.3.6 | ACOX1; ACOX3 | −0.04 |
| EC: 1.3.3.6 | ACOX1; ACOX3 | −0.04 |
| EC: 1.3.3.6 | ACOX1; ACOX3 | −0.04 |
| PA6 exchange | | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| Y+LAT2 Utilized transport | SLC7A6 | −0.04 |
| UDP-N-acetylglucosamine 4-epimerase | GALE | −0.04 |
| spermine monoaldehyde exchange | | −0.04 |
| RE0688 | AOC1 | −0.04 |
| 5,10-methylenetetrahydrofolatereductase | MTHFR | −0.04 |
| (NADPH) | | |
| 5-methyltetrahydrofolate: NAD+ | MTHFR | −0.04 |
| oxidoreductase One carbon pool by folate/ | | |
| Methane metabolism EC: 1.5.1.20 | | |
| blood group intracellular transport | | −0.04 |
| blood group intracellular transport | | −0.04 |
| VI3NeuAc-nLc6Cer exchange | | −0.04 |
| Type 2 lactosamine alpha-2,3- | ST3GAL6 | −0.04 |
| sialyltransferase | | |
| Noradrenaline secretion via secretory vesicle | SLC18A1; | −0.04 |
| (ATP driven) | SLC18A2 | |
| Beta oxidation fatty acid | ACADM; ACADS | −0.04 |
| Beta oxidation fatty acid | ACADM; ACADS | −0.04 |
| Hexadecanoate (n-C16: 0) exchange | | −0.04 |
| RE0344 | ACOT2; ACOT6; | −0.04 |
| | BAAT | |
| hypothetical enzyme | | −0.04 |
| Pyridoxine 5-phosphate phosphatase | | −0.04 |
| pyridoxamine kinase | PDXK | −0.04 |
| pyridoxal kinase | PDXK | −0.04 |
| pyridoxine kinase | PDXK | −0.04 |
| Pyridoxal 5-phosphate phosphatase | | −0.04 |
| Hydrogen peroxide exchange | | −0.04 |
| hydrogen peroxide transport via diffusion | | −0.04 |
| L-cysteine transport via diffusion | SLC43A2 | −0.04 |
| (extracellular to cytosol) | | |
| L-isoleucine transport via diffusion | SLC43A1; | −0.04 |
| (extracellular to cytosol) | SLC43A2 | |
| L-methionine transport via diffusion | SLC43A1; | −0.04 |
| (extracellular to cytosol) | SLC43A2 | |
| L-valine transport via diffusion (extracellular | SLC43A1; | −0.04 |
| to cytosol) | SLC43A2 | |
| Transport reaction | | −0.04 |
| uridylate kinase (dUMP), mitochondrial | DTYMK | −0.04 |
| RE0691 | | −0.04 |
| RE1897 | | −0.04 |
| RE1898 | | −0.04 |
| exchange reaction for Uridine | | −0.04 |
| keratan sulfate I exchange | | −0.04 |
| PA6 exchange | | −0.04 |
| Exchange of Flavin adenine dinucleotide | | −0.04 |
| oxidized | | |
| Exchange of alpha-D-glucose 1-phosphate(2-) | | −0.04 |
| Exchange of [(2R,3S,4R,5R)-5-(2,4-dioxo- | | −0.04 |
| 1,2,3,4-tetrahydropyrimidin-1-yl)-3,4- | | |
| dihydroxyoxolan-2-yl]methyl | | |
| {[(3R,4S,5S,6R)-3,4,5-trihydroxy-6- | | |
| (hydroxymethyl)oxan-2-yl | | |
| phosphonato]oxy}phosphonate | | |
| UDPglucose pyrophosphohydrolase | ENPP1; ENPP3 | −0.04 |
| Pyrimidine metabolism/Starch and sucrose | | |
| metabolism EC: 3.6.1.45 | | |
| carnitine C20: 4 transferase | CPT1A; CPT1B; | −0.04 |
| | CPT1C | |
| arachidonic acid transport into the | CPT2 | −0.04 |
| mitochondria | | |
| arachidonic acid transport into the | SLC25A20 | −0.04 |
| mitochondria | | |
| Beta oxidation of long chain fatty acid | ACADM; ACADS | −0.04 |
| Palmitoyl-CoA: L-carnitine O- | CPT1A; CPT2 | −0.04 |
| palmitoyltransferase Fatty acid metabolism | | |
| EC: 2.3.1.21 | | |
| Palmitoyl-CoA: L-carnitine O- | CPT1A; CPT2 | −0.04 |
| palmitoyltransferase Fatty acid metabolism | | |
| EC: 2.3.1.21 | | |
| Facilitated diffusion | | −0.04 |
| Facilitated diffusion | | −0.04 |
| EC: 6.2.1.3 | ACSBG2; ACSL1; | −0.04 |
| | ACSL3; ACSL4; | |
| | ACSL5; ACSL6; | |
| | SLC27A2 | |
| 3-(4-hydroxyphenyl-)lactate formation | | −0.04 |
| Hydroxylation of 3-Decaprenyl-4- | | −0.04 |
| hydroxybenzoate (NADH) | | |
| Hydroxylation of 3-Decaprenyl-4- | | −0.04 |
| hydroxybenzoate (NADPH) | | |
| 4-hydroxybenzoyl-CoA formation | | −0.04 |
| 4-hydroxybenzoate formation | | −0.04 |
| methyltransferase COQ3 | COQ3 | −0.04 |
| Ubiquinone biosynthesis methyltransferase | | −0.04 |
| COQ5 | | |
| Ubiquinone biosynthesis monooxgenase | COQ6 | −0.04 |
| COQ6 | | |
| Ubiquinone biosynthesis COQ7 | COQ7 | −0.04 |
| p-coumaroyl-CoA formation | | −0.04 |
| lipid, flip-flop intracellular transport | | −0.04 |
| dihydroxydecaprenylbenzoate | COQ3 | −0.04 |
| methyltransferase | | |
| 3-Decaprenyl-4-hydroxy-5-methoxybenzoate | | −0.04 |
| decarboxylation | | |
| decaprenyl synthase | | −0.04 |
| Hydroxybenzoate Decaprenyltransferase | COQ2 | −0.04 |
| Vesicular transport | | −0.04 |
| 4-hydroxycinnamate formation | | −0.04 |
| tyrosine transaminase, mitochondrial | GOT2; TAT | −0.04 |
| L-proline reversible transport via proton | SLC36A1; | −0.04 |
| symport | SLC36A2 | |
| carnitine O-palmitoyltransferase | CPT1A; CPT1B; | −0.04 |
| | CPT1C | |
| carnitine transferase | CPT2 | −0.04 |
| transport into the mitochondria (carnitine) | SLC25A20 | −0.04 |
| Beta oxidation of fatty acid | ACADM; ACADS | −0.04 |
| Hydrogen peroxide exchange | | −0.04 |
| prostaglandin I2(1-) exchange | | −0.04 |
| Thromboxane A2 exchange | | −0.04 |
| Prostaglandin I2 synthase | PTGIS | −0.04 |
| Prostaglandin G/H synthase | PTGS1; PTGS2 | −0.04 |
| Prostaglandin I2 transport (ER) | | −0.04 |
| thromboxane A2 transport | | −0.04 |
| thromboxane A2 intracellular transport | | −0.04 |
| Thromboxane-A synthase | TBXAS1 | −0.04 |
| fatty acid intracellular transport | | −0.04 |
| nC22: 6 exchange | | −0.04 |
| fatty-acid--CoA ligase | ACSL1 | −0.04 |
| Propane-1,2-diol: NADP+ 1-oxidoreductase | AKR1A1; AKR1B3; | −0.04 |
| | AKR7A5 | |
| xenobiotic transport | | −0.04 |
| xenobiotic transport | | −0.04 |
| antipyrene exchange | | −0.04 |
| demethylated antipyrine exchange | | −0.04 |
| cytochrome P450 2C18 | CYP2C55 | −0.04 |
| methylenetetrahydrofolate dehydrogenase | MTHFD1; MTHFR | −0.04 |
| (NADP) | | |
| methylenetetrahydrofolate dehydrogenase | MTHFD2; | −0.04 |
| (NAD) | MTHFD2L | |
| D-Glucose exchange | | −0.04 |
| 5′-nucleotidase (CMP) | NT5C; NT5C1A; | −0.05 |
| | NT5C1B; NT5C2; | |
| | NT5C3; NT5E | |
| dUTP: cytidine 5-phosphotransferase | UCK1; UCK2 | −0.05 |
| Pyrimidine metabolism EC: 2.7.1.48 | | |
| nucleoside-diphosphate kinase (ATP: UDP), | NME4 | −0.05 |
| mitochondrial | | |
| 5′-nucleotidase (UMP), mitochondrial | NT5M | −0.05 |
| RE0456 | | −0.05 |
| UMP kinase (mitochondrial, ATP) | | −0.05 |
| uridine facilated transport in mitochondria | SLC29A1 | −0.05 |
| chenodeoxycholate exchange | | −0.05 |
| exchange reaction for 5- | | −0.05 |
| Methyltetrahydrofolate | | |
| fatty acid intracellular transport | | −0.05 |
| fatty acyl-CoA desaturase (n-C22: 5CoA −> n- | | −0.05 |
| C22: 6CoA) | | |
| fatty-acyl-CoA elongation (n-C20: 5CoA) | ELOVL2; EL0VL4; | −0.05 |
| | ELOVL5; ELOVL6 | |
| Beta oxidation of long chain fatty acid | ACAA1B; ACOX1; | −0.05 |
| | EHHADH; | |
| | HSD17B4 | |
| fatty acid intracellular transport | | −0.05 |
| IDP exchange | | −0.05 |
| IMP exchange | | −0.05 |
| nucleoside-diphosphatase (IDP), | CANT1; ENTPD1; | −0.05 |
| extracellular | ENTPD3; ENTPD8 | |
| dihydrofolate reversible mitochondrial | | −0.05 |
| transport | | |
| Dihydrofolate: NADP+ oxidoreductase One | DHFR | −0.05 |
| carbon pool by folate/Folate biosynthesis | | |
| EC: 1.5.1.3 | | |
| Facilitated diffusion | | −0.05 |
| diffusion of putriscine into the endothelial | | −0.05 |
| cells | | |
| 2-Oxoadipate: lipoamde 2- | DLD; DLST; OGDH; | −0.05 |
| oxidoreductase(decarboxylating and | PDHX | |
| acceptor-succinylating) (mitochondria) | | |
| L-lysine transport via diffusion (extracellular | SLC38A4; SLC7A1; | −0.05 |
| to cytosol) | SLC7A2; SLC7A3 | |
| ornithine transport via diffusion | SLC7A1; SLC7A2; | −0.05 |
| (extracellular to cytosol) | SLC7A3 | |
| L-arginine transport via diffusion | SLC38A4; SLC7A1; | −0.05 |
| (extracellular to cytosol) | SLC7A2; SLC7A3 | |
| de-Fuc form of PA6 exchange | | −0.05 |
| Exchange of 4-ammoniobutanal | | −0.05 |
| Putrescine: oxygen oxidoreductase | AOC1 | −0.05 |
| (deaminating) Urea cycle and metabolism of | | |
| amino groups EC: 1.4.3.6 | | |
| Transport of L-Histidine into the cell coupled | SLC38A5 | −0.05 |
| with co-transport with Sodium and counter | | |
| transport with proton by SNAT5 transporter. | | |
| Butyrate mitochondrial transport via proton | SLC16A1 | −0.05 |
| symport, reversible | | |
| Butyrate (n-C4: 0) exchange | | −0.05 |
| fatty-acid--CoA ligase (butanoate), | ACSM1 | −0.05 |
| mitochondrial | | |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| cholesterol precursor intracellular transport | | −0.05 |
| C-3 sterol dehydrogenase (4- | NSDHL | −0.05 |
| methylzymosterol) | | |
| C-3 sterol dehydrogenase (4- | NSDHL | −0.05 |
| methylzymosterol) | | |
| C-3 sterol keto reductase (zymosterol) | HSD17B4 | −0.05 |
| C-4 sterol methyl oxidase (4,4- | MSMO1 | −0.05 |
| dimethylzymosterol) | | |
| C-4 methyl sterol oxidase | NSDHL | −0.05 |
| C-4 methyl sterol oxidase | MSMO1; NSDHL | −0.05 |
| 24-dehydrocholesterol reductase | DHCR24 | −0.05 |
| [Precursor] | | |
| 7-dehydrocholesterol reductase | DHCR7 | −0.05 |
| dimethylallyltranstransferase | GGPS1 | −0.05 |
| dimethylallyltranstransferase | FDPS | −0.05 |
| Desmosterol reductase | DHCR24 | −0.05 |
| geranyltranstransferase | GGPS1 | −0.05 |
| geranyltranstransferase | FDPS | −0.05 |
| isopentenyl-diphosphate D-isomerase | GM9745; IDI1 | −0.05 |
| isopentenyl-diphosphate D-isomerase | GM9745; IDI1 | −0.05 |
| lanosterol synthase | LSS | −0.05 |
| Lathosterol oxidase | SC5D | −0.05 |
| Lathosterol oxidase | CYP7A1; SC5D | −0.05 |
| Previtamin D3 formation | | −0.05 |
| Farnesyl-diphosphate: farnesyl-diphosphate | FDFT1 | −0.05 |
| farnesyltransferase Biosynthesis of steroids | | |
| EC: 2.5.1.21 | | |
| Presqualene diphosphate: farnesyl- | FDFT1 | −0.05 |
| diphosphate farnesyltransferase | | |
| Biosynthesis of steroids EC: 2.5.1.21 | | |
| 4,4-dimethyl-5a-cholesta-8,24-dien-3b- | TM7SF2 | −0.05 |
| ol: NADP+ D14-oxidoreductase Biosynthesis | | |
| of steroids EC: 1.3.1.70 | | |
| Lanosterol, NADPH: oxygen oxidoreductase | CYP51 | −0.05 |
| (14-methyl cleaving) Biosynthesis of steroids | | |
| EC: 1.14.13.70 | | |
| delta24-sterol reductase Biosynthesis of | DHCR24 | −0.05 |
| steroids EC: 1.3.1.72 | | |
| 5alpha-Cholest-7-en-3beta-ol delta7-delta8- | EBP | −0.05 |
| isomerase Biosynthesis of steroids EC: 5.3.3.5 | | |
| Squalene epoxidase, endoplasmic reticular | SQLE | −0.05 |
| (NADP) | | |
| Vitamin D3 formation | | −0.05 |
| 5′-nucleotidase (dUMP) | NT5C; NT5C3 | −0.05 |
| 5′-nucleotidase (dTMP) | NT5C; NT5C1A; | −0.05 |
| | NT5C1B; NT5C2; | |
| | NT5C3; NT5E | |
| Sarcosine exchange | | −0.05 |
| Sarcosine transport (extracellular to cytosol) | | −0.05 |
| fatty acid transport via diffusion | SLC27A5 | −0.05 |
| dihomo-gamma-linolenic acid (n-6) | | −0.05 |
| exchange | | |
| fatty-acid--CoA ligase | ACSL1 | −0.05 |
| Utilized transport | | −0.05 |
| UDP-Glc endoplasmic reticulum transport via | | −0.05 |
| CMP antiport | | |
| 3-hydroxyanthranilate 3,4-dioxygenase | HAAO | −0.05 |
| 2-aminomuconate reductase | | −0.05 |
| 3-Hydroxy-L-kynurenine hydrolase | KYNU | −0.05 |
| picolinic acid decarboxylase | ACMSD | −0.05 |
| 2-Aminomuconate semialdehyde: NAD+ 6- | | −0.05 |
| oxidoreductase Tryptophan metabolism | | |
| EC: 1.2.1.32 | | |
| alcohol dehydrogenase, forward rxn | ADH1; ADH4; | −0.05 |
| (ethanol −> acetaldehyde) | ADH5; ADH6A; | |
| | ADH7; ADHFE1; | |
| | ZADH2 | |
| methanol exchange | | −0.05 |
| Methanol diffusion | | −0.05 |
| 13-cis-retinoic acid isomerase | | −0.05 |
| R total 2 position exchange | | −0.05 |
| fatty acyl-CoA desaturase (n-C20: 3CoA −> n- | FADS1 | −0.05 |
| C20: 4CoA) | | |
| 3-amino-isobutyrate transport | | −0.05 |
| 3-amino-isobutyrate transport, | ABAT | −0.05 |
| mitochondrial | | |
| L-3-aminoisobutyrate transaminase, | ABAT | −0.05 |
| mitochondrial | | |
| L-3-Amino-isobutanoate exchange | | −0.05 |
| Y+LAT2 Utilized transport | SLC7A6 | −0.05 |
| deoyguanosine transport via diffusion | SLC29A2 | −0.05 |
| deoxyinosine transport via diffusion | SLC29A2 | −0.05 |
| Concentrative Nucleoside Transporter (CNT) | SLC28A2; | −0.05 |
| TCDB: 2.A.41.2.4 | SLC28A3 | |
| Concentrative Nucleoside Transporter (CNT) | SLC28A2; | −0.05 |
| TCDB: 2.A.41.2.4 | SLC28A3 | |
| Facilitated diffusion | | −0.05 |
| leukotriene D4 exchange | | −0.05 |
| leukotriene E4 exchange | | −0.05 |
| Gamma-glutamyltransferase 5 | GGT5 | −0.05 |
| intracellular transport | | −0.05 |
| glutathione transport via diffusion | | −0.05 |
| leukotriene intracellular transport | | −0.05 |
| leukotriene intracellular transport | | −0.05 |
| Leukotriene C4 synthase | LTC4S; MGST2; | −0.05 |
| | MGST3 | |
| D-Glyceraldehyde: NAD+ oxidoreductase | ALDH1B1; | −0.05 |
| Glycerolipid metabolism EC: 1.2.1.3 | ALDH2; | |
| | ALDH3A2; | |
| | ALDH7A1; | |
| | ALDH9A1 | |
| exchange reaction for NO | | −0.05 |
| L-Arginine, NADPH: oxygen oxidoreductase | NOS1; NOS2; | −0.05 |
| (nitric-oxide-forming) Arginine and proline | NOS3 | |
| metabolism EC: 1.14.13.39 | | |
| production of Palmitoylcarnitine | CPT1A; CPT1B; | −0.05 |
| | CPT1C | |
| fatty acid intracellular transport | | −0.05 |
| Palmitoyl-CoA: L-carnitine O- | CPT1A; CPT2 | −0.05 |
| palmitoyltransferase Fatty acid metabolism | | |
| EC: 2.3.1.21 | | |
| Palmitoyl-CoA: L-carnitine O- | CPT1A; CPT2 | −0.05 |
| palmitoyltransferase Fatty acid metabolism | | |
| EC: 2.3.1.21 | | |
| Facilitated diffusion | | −0.05 |
| Facilitated diffusion | | −0.05 |
| 5-Methyltetrahydrofolate transport via | FOLR1 | −0.05 |
| receptor binding and protolysis | | |
| Sulfate exchange | | −0.05 |
| sphingosylphosphorylcholine | | −0.05 |
| (homo sapiens) exchange | | |
| sphingosylphosphorylcholine transport | | −0.05 |
| (diffusion) | | |
| glucose-6-phosphate phosphatase, | G6PC; G6PC2; | −0.05 |
| edoplasmic reticular | G6PC3 | |
| glucose transport, endoplasmic reticulum | | −0.05 |
| H2O endoplasmic reticulum transport | | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| D-sorbitol dehydrogenase (D-fructose | SORD | −0.05 |
| producing) | | |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Vitamin D3 release | | −0.05 |
| Vitamin D3 uptake | CD36; GOT2 | −0.05 |
| RE2973 | PIK3C2A; | −0.05 |
| | PIK3C2B; | |
| | PIK3C2G | |
| RE2974 | | −0.05 |
| transport of L-Cysteine into the cell coupled | SLC38A5 | −0.05 |
| with co-transport with Sodium and counter | | |
| transport with proton by SNAT5 transporter. | | |
| L-ascorbate transport via proton symport | SLC23A1; | −0.05 |
| | SLC23A2 | |
| ATP: dephospho-CoA 3-phosphotransferase | COASY | −0.05 |
| Pantothenate and CoA biosynthesis | | |
| EC: 2.7.1.24 | | |
| ATP: pantetheine-4-phosphate | COASY | −0.05 |
| adenylyltransferase Pantothenate and CoA | | |
| biosynthesis EC: 2.7.7.3 | | |
| phosphoglycerate kinase | MIA3; PGK1; PGK2 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| cysteinesulfinic acid oxidase | | −0.05 |
| exchange reaction for L-asparagine | | −0.05 |
| 5-beta-cholestane-3-alpha,7-alpha,12-alpha- | CYP27A1 | −0.05 |
| triol 27-hydroxylase | | |
| 5-beta-cholestane-3-alpha,7-alpha,12-alpha- | CYP27A1 | −0.05 |
| triol 27-hydroxylase | | |
| RE2625 | CYP27A1 | −0.05 |
| Major Facilitator(MFS) TCDB: 2.A.1.44.1 | SLC43A1 | −0.05 |
| difussion of glycerol accross the brush | | −0.05 |
| border membrane | | |
| spermine dialdehyde exchange | | −0.05 |
| RE0827 | AOC1 | −0.05 |
| retinol isomerase (9-cis) | | −0.05 |
| retinal isomerase (9-cis) | | −0.05 |
| retinol isomerase (11-cis) | | −0.05 |
| retinol dehydrogenase (all-trans) | RDH16F1; RDH5 | −0.05 |
| retinol dehydrogenase (all-trans, NADPH) | RDH10; RDH11; | −0.05 |
| | RDH12; RDH13; | |
| | RDH14; RDH8; | |
| | SDR16C5 | |
| retinal isomerase (11-cis) | | −0.05 |
| adenosine facilated transport in | SLC29A1 | −0.05 |
| mitochondria | | |
| fatty acid beta oxidation(C10: 1-->C8)m | ACAA2; ECHS1; | −0.05 |
| | HADH | |
| Octanoyl-CoA: acetyl-CoA C-acyltransferase | ACAA1B; ACAA2; | −0.05 |
| Fatty acid elongation in mitochondria/Fatty | HADHB | |
| acid metabolism EC: 2.3.1.16 | | |
| (S)-Hydroxydecanoyl-CoA: NAD+ | EHHADH; HADH; | −0.05 |
| oxidoreductase Fatty acid elongation in | HADHA | |
| mitochondria/Fatty acid metabolism | | |
| EC: 1.1.1.35 EC: 1.1.1.211 | | |
| (S)-Hydroxydecanoyl-CoA hydro-lyase Fatty | ECHS1; EHHADH; | −0.05 |
| acid elongation in mitochondria/Fatty acid | HADHA | |
| metabolism EC: 4.2.1.17 | | |
| thiamine exit from the neterocytes | | −0.05 |
| Thiamine transport in via proton antiport | SLC19A2; | −0.05 |
| | SLC19A3 | |
| D-alanine transport via proton symport | SLC36A1 | −0.05 |
| RE2649 | ACOT2; ACOT6; | −0.05 |
| | BAAT | |
| lactaldehyde dehydrogenase | ALDH1A1; | −0.05 |
| | ALDH1A2; | |
| | ALDH1A3; | |
| | ALDH3A1; | |
| | ALDH3A2; | |
| | ALDH3B1; | |
| | ALDH3B3; | |
| | ALDH7A1; | |
| | ALDH9A1 | |
| cytidine deaminase, nuclear | AICDA | −0.05 |
| cytidine transport in nucleus | | −0.05 |
| uridine transport in nucleus | | −0.05 |
| glycine reversible transport via sodium and | SLC6A9 | −0.05 |
| chloride symport (2:1:1) | | |
| transport of succinyl carnitine into cytosol | SLC25A20 | −0.05 |
| fatty acid beta oxidation(C6DC-->C4DC)x | ACAA1B; ACOX1; | −0.05 |
| | EHHADH; | |
| | HSD17B4 | |
| production of succinyl carnitine | CROT | −0.05 |
| 2-oxoglutarate dehydrogenase E1 | OGDH | −0.05 |
| component Citrate cycle (TCA cycle) | | |
| EC: 1.2.4.2 | | |
| 2-oxoglutarate: [dihydrolipoyllysine-residue | OGDH | −0.05 |
| succinyltransferase]-lipoyllysine 2- | | |
| oxidoreductase (decarboxylating, acceptor- | | |
| succinylating) EC: 1.2.4.2 | | |
| succinyl-CoA: enzyme N6- | DLST | −0.05 |
| (dihydrolipoyl)lysine S-succinyltransferase | | |
| Citrate cycle (TCA cycle) EC: 2.3.1.61 | | |
| 2-oxoglutarate dehydrogenase E1 | OGDH | −0.05 |
| component Citrate cycle (TCA cycle) | | |
| EC: 1.2.4.2 | | |
| fatty acyl-CoA desaturase (n-C20: 4CoA −> n- | FADS1 | −0.05 |
| C20: 5CoA) | | |
| Beta oxidation of med/long chain fatty acid | ACADM; ACADS | −0.05 |
| Ammonia exchange | | −0.05 |
| phosphate transport, nuclear | | −0.05 |
| glucose 6-phosphate endoplasmic reticular | SLC37A4 | −0.05 |
| transport via diffusion | | |
| hypoxanthine diffusion in peroxisome | | −0.05 |
| xanthine oxidase | XDH | −0.05 |
| sulfonated testosterone transport | | −0.05 |
| 5alpha-Dihydrotestosterone sulfate | | −0.05 |
| exchange | | |
| RE3218 | | −0.05 |
| Transport of L-Leucine into the intestinal | SLC6A14 | −0.05 |
| cells by ATBO transporter | | |
| L-Arginine exchange | | −0.05 |
| glutamate dehydrogenase (NAD) | GLUD1 | −0.05 |
| (mitochondrial) | | |
| glutamate dehydrogenase (NADP), | GLUD1 | −0.05 |
| mitochondrial | | |
| glutamate dehydrogenase (NAD) | GLUD1 | −0.05 |
| (mitochondrial) | | |
| glutamate dehydrogenase (NADP), | GLUD1 | −0.05 |
| mitochondrial | | |
| Y+LAT2 Utilized transport | SLC7A6 | −0.05 |
| Y+LAT2 Utilized transport | SLC7A6 | −0.05 |
| Y+LAT2 Utilized transport | SLC7A6 | −0.05 |
| guanylate cyclase | GUCY1A2; | −0.05 |
| | GUCY1A3; | |
| | GUCY1B3; | |
| | GUCY2C; GUCY2E; | |
| | GUCY2F; NPR1; | |
| | NPR2 | |
| 3′,5′-cyclic-nucleotide phosphodiesterase | PDE10A; PDE11A; | −0.05 |
| | PDE1A; PDE1B; | |
| | PDE1C; PDE2A; | |
| | PDE3A; PDE3B; | |
| | PDE5A; PDE6A; | |
| | PDE6B; PDE6C; | |
| | PDE6D; PDE6G; | |
| | PDE6H; PDE9A | |
| transport of dGTP into mitochondria | | −0.05 |
| L-leucine transport in via sodium symport | ACE2; SLC38A1; | −0.05 |
| | SLC38A4; SLC3A2; | |
| | SLC6A14; | |
| | SLC6A19; SLC7A1; | |
| | SLC7A2; SLC7A3; | |
| | TMEM27 | |
| adenylate kinase, mitochondrial | AK2; AK4 | −0.05 |
| adenylate kinase, mitochondrial | AK2; AK4 | −0.05 |
| adentylate kinase (GTP) | AK3 | −0.05 |
| adentylate kinase (GTP) | AK3 | −0.05 |
| Amino Acid-Polyamine-Organocation (APC) | SLC7A2 | −0.05 |
| TCDB: 2.A.3.3.2 | | |
| Prostaglandin D2 exchange | | −0.05 |
| Prostaglandin-H2 D-isomerase [Precursor] | HPGDS; PTGDS | −0.05 |
| prostaglandin H2(1-) exchange | | −0.05 |
| Y+LAT2 Utilized transport | SLC7A6 | −0.05 |
| UDPglucose 4-epimerase | GALE | −0.05 |
| alanine racemase | PROSC | −0.05 |
| D-Alanine exchange | | −0.05 |
| exchange reaction for L-alanine | | −0.05 |
| calcium/sodium antiporter (1:3), reversible | SLC8A1; SLC8A2; | −0.06 |
| | SLC8A3 | |
| 3′,5′-bisphosphate nucleotidase | BPNT1 | −0.06 |
| steroid sulfotransferase | SULT1A1; | −0.06 |
| | SULT2B1 | |
| steroid sulfotransferase | SULT1A1; | −0.06 |
| | SULT2A1; | |
| | SULT2B1 | |
| RE1100 | | −0.06 |
| RE1135 | | −0.06 |
| sulfate adenylyltransferase | PAPSS1; PAPSS2 | −0.06 |
| Y+LAT2 Utilized transport | SLC7A6 | −0.06 |
| Y+LAT2 Utilized transport | SLC7A6 | −0.06 |
| Y+LAT2 Utilized transport | SLC7A6 | −0.06 |
| Y+LAT2 Utilized transport | SLC7A6 | −0.06 |
| Y+LAT2 Utilized transport | SLC7A6 | −0.06 |
| Phosphatidylserine decarboxylase | | −0.06 |
| phosphatidylserine flippase | ATP10A; ATP8A1 | −0.06 |
| L-Lactate dehydrogenase, | CYB5D1 | −0.06 |
| cytosolic/mitochondrial | | |
| Succinate exchange | | −0.06 |
| succinate transport via sodium symport | SLC13A2 | −0.06 |
| Prostaglandin G/H synthase | PTGS1; PTGS2 | −0.06 |
| L-Tryptophan, tetrahydrobiopterin: oxygen | TPH1; TPH2 | −0.06 |
| oxidoreductase (5-hydroxylating) | | |
| dolichol diffusion, human (liver) | | −0.06 |
| dolichol diffusion, human (uterus) | | −0.06 |
| Dolichol kinase, human (liver) | | −0.06 |
| Dolichol kinase, human (uterus) | | −0.06 |
| dolichyl-phosphate-glucose-glycolipid alpha- | ALG10B | −0.06 |
| glucosyltransferase (liver) | | |
| dolichyl-phosphate-glucose-glycolipid alpha- | ALG10B | −0.06 |
| glucosyltransferase (uterus) | | |
| retinal dehydrogenase (NADPH) | | −0.06 |
| retinol dehydrogenase (13-cis, NADH) | RDH5 | −0.06 |
| retinol isomerase (13-cis) | | −0.06 |
| hydrogen peroxide transport via diffusion | | −0.06 |
| H2O exchange | | −0.06 |
| retinol isomerase (9-cis) | | −0.06 |
| retinol dehydrogenase (9-cis, NADH) | RDH5 | −0.06 |
| retinol dehydrogenase (9-cis, NADPH) | RDH11; RDH12; | −0.06 |
| | RDH13; RDH14; | |
| | RDH8; SDR16C5 | |
| retinol dehydrogenase (11-cis, NADH) | RDH5 | −0.06 |
| retinol dehydrogenase (11-cis, NADPH) | RDH11; RDH12; | −0.06 |
| | RDH13; RDH14; | |
| | SDR16C5 | |
| retinal isomerase (11-cis) | | −0.06 |
| retinol isomerase (11-cis) | | −0.06 |
| retinal isomerase (9-cis) | | −0.06 |
| Reduced glutathione exchange | | −0.06 |
| fatty acid transport via diffusion | | −0.06 |
| eicosatetranoic acid exchange | | −0.06 |
| fatty-acid--CoA ligase | ACSL1 | −0.06 |
| fatty-acyl-CoA elongation (n-C20: 4CoA) | ELOVL2; EL0VL4; | −0.06 |
| | ELOVL5; ELOVL6 | |
| 4-Hydroxyphenylacetaldehyde: NADP+ | ALDH1A3; | −0.06 |
| oxidoreductase | ALDH3A1; | |
| | ALDH3B1; | |
| | ALDH3B3 | |
| 4-Hydroxyphenylacetate exchange | | −0.06 |
| hydroxyphenylacetate transport via diffusion | | −0.06 |
| L-Tyrosine carboxy-lyase | DDC | −0.06 |
| Tyramine: oxygen | MAOA; MAOB | −0.06 |
| oxidoreductase(deaminating)(flavin- | | |
| containing) (cytosol) | | |
| 7-alpha,24(S)-Dihydroxycholesterol | | −0.06 |
| exchange | | |
| 7-alpha,25-Dihydroxycholesterol exchange | | −0.06 |
| oxysterol 7-alpha-hydroxylase | CYP39A1 | −0.06 |
| cytochrome P450, family 46, subfamily A, | CYP46A1 | −0.06 |
| polypeptide 1 | | |
| oxysterol 7alpha-hydroxylase | CYP7B1 | −0.06 |
| Cholest-5-ene-3beta,7alpha-diol: NAD+ 3- | HSD3B7 | −0.06 |
| oxidoreductase Bile acid biosynthesis | | |
| EC: 1.1.1.181 | | |
| 24 trihydroxy cholesterol transport | | −0.06 |
| 24 trihydroxy cholesterol transport | | −0.06 |
| 25 trihydroxy cholesterol transport | | −0.06 |
| 25 trihydroxy cholesterol transport | | −0.06 |
| ammonia peroxisomal transport | | −0.06 |
| acetyl-CoA C-acetyltransferase | ACAT3 | −0.06 |
| fatty acyl-CoA desaturase (n-C18: 1CoA −> n- | FADS2 | −0.06 |
| C18: 2CoA) | | |
| L-Aspartate exchange | | −0.06 |
| O2 transport, endoplasmic reticulum | | −0.06 |
| D-aspartate transport, extracellular | | −0.06 |
| D-aspartate transport, peroxisomal | | −0.06 |
| D-aspartate oxidase, peroxisomal | DDO | −0.06 |
| D-Aspartate exchange | | −0.06 |
| Transport reaction for malate | | −0.06 |
| malate dehydrogenase, peroxisomal | | −0.06 |
| isomerization(C10: 1)m | ECU | −0.06 |
| fatty acid beta oxidation(C12: 1-->C10: 1)m | ACAA2; ACADM; | −0.06 |
| | ECHS1; HADH | |
| fatty acid beta oxidation(C14: 1-->C12: 1)m | ACADVL; HADHA; | −0.06 |
| | HADHB | |
| Lactosylceramide 4-alpha- | A4GALT | −0.06 |
| galactosyltransferase | | |
| aegagbside hs intracellular transport | | −0.06 |
| aegagbside hs intracellular transport | | −0.06 |
| Beta-1,3-galactosyltransferase 3 | B3GALNT1 | −0.06 |
| globoside (homo sapiens) exchange | | −0.06 |
| globoside alpha-1,3-N- | GBGT1 | −0.06 |
| acetylgalactosaminyltransferase 1 | | |
| globoside transport | | −0.06 |
| globoside intracellular transport | | −0.06 |
| N-acetylgalactosaminidase, alpha- | NAGA | −0.06 |
| UDP-GalNAc Golgi transport via CMP | SLC35A2 | −0.06 |
| antiport | | |
| Y+LAT2 Utilized transport | SLC7A6 | −0.06 |
| Y+LAT2 Utilized transport | SLC7A6 | −0.06 |
| Alanine-Sodium symporter | ACE2; SLC38A1; | −0.06 |
| | SLC38A2; | |
| | SLC38A4; | |
| | SLC6A14; | |
| | SLC6A19; SLC7A1; | |
| | SLC7A2; SLC7A3; | |
| | TMEM27 | |
| exchange reaction for glycylphenylalaine | | −0.06 |
| hydrolysis of glycylphenylalanine | | −0.06 |
| transport of Glycylphenylalanine by the | SLC15A1 | −0.06 |
| apical PEPT1 amino acid transporters across | | |
| the brush border cells of the enterocytes of | | |
| the intestine and renal cells | | |
| Y+LAT2 Utilized transport | SLC7A6 | −0.06 |
| Y+LAT2 Utilized transport | SLC7A6 | −0.06 |
| Y+LAT2 Utilized transport | SLC7A6 | −0.06 |
| Y+LAT2 Utilized transport | SLC7A6 | −0.06 |
| Y+LAT2 Utilized transport | SLC7A6 | −0.06 |
| Y+LAT2 Utilized transport | SLC7A6 | −0.06 |
| Y+LAT2 Utilized transport | SLC7A6 | −0.06 |
| Y+LAT2 Utilized transport | SLC7A6 | −0.06 |
| Y+LAT2 Utilized transport | SLC7A6 | −0.06 |
| 35cGMP nuclear transport | | −0.06 |
| GMP nuclear transport | | −0.06 |
| 3′,5′-cyclic-nucleotide phosphodiesterase, | PDE9A | −0.06 |
| Nucleus | | |
| 5-Methyltetrahydrofolate transport via | FOLR1 | −0.06 |
| receptor binding and protolysis | | |
| 5-methyltetrahydrofolate transport via anion | SLC19A1 | −0.06 |
| exchange | | |
| exchange reaction for proton | | −0.06 |
| Progesterone exchange | | −0.06 |
| Progesterone transport | | −0.06 |
| fatty acid beta oxidation(C16-->C14)x | ACAA1B; ACOX1; | −0.06 |
| | EHHADH; | |
| | HSD17B4 | |
| Glutamate Decarboxylase | GAD1; GAD2 | −0.06 |
| carnitine O-palmitoyltransferase | CPT1A; CPT1B; | −0.06 |
| | CPT1C | |
| carnitine transferase | CPT2 | −0.06 |
| transport into the mitochondria (carnitine) | SLC25A20 | −0.06 |
| Beta oxidation of long chain fatty acid | ACADM; ACADS | −0.06 |
| nicotinate-nucleotide adenylyltransferase | NMNAT1; | −0.06 |
| | NMNAT2; | |
| | NMNAT3 | |
| Deamino-NAD+ nucleotidohydrolase | ENPP1; ENPP3; | −0.06 |
| Nicotinate and nicotinamide metabolism | NUDT12 | |
| EC: 3.6.1.9 | | |
| 5-methyltetrahydrofolate: NAD+ | MTHFR | −0.06 |
| oxidoreductase One carbon pool by folate/ | | |
| Methane metabolism EC: 1.5.1.20 | | |
| L-homoserine via sodium symport | SLC7A1; SLC7A2; | −0.06 |
| | SLC7A3 | |
| Mitochondrial Carrier (MC) TCDB: 2.A.29.7.2 | SLC25A1 | −0.06 |
| Mitochondria] Carrier (MC) TCDB: 2.A.29.7.2 | SLC25A1 | −0.06 |
| transport of Glycine into the cell coupled | SLC38A5 | −0.06 |
| with co-transport with Sodium and counter | | |
| transport with proton by SNAT5 transporter. | | |
| Y+LAT2 Utilized transport | SLC7A6 | −0.06 |
| Y+LAT2 Utilized transport | SLC7A6 | −0.06 |
| Y+LAT2 Utilized transport | SLC7A6 | −0.06 |
| 2-oxoadipate shuttle (cytosol/mitochondria) | SLC25A21 | −0.06 |
| 24,25-Dihydroxyvitamin D2 transport from | | −0.06 |
| cytoplasm | | |
| 24,25-Dihydroxyvitamin D2 transport from | | −0.06 |
| mitochondria | | |
| 24R-Vitamin D-25-hydroxylase (D2) | CYP24A1 | −0.06 |
| 24R,25-Dihyoxyvitamin D2 exchange | | −0.06 |
| 25-Hydroxyvitamin D2 exchange | | −0.06 |
| 5-methyltetrahydrofolate transport via anion | SLC19A1 | −0.06 |
| exchange | | |
| 5-methyltetrahydrofolate transport via anion | | −0.06 |
| exchange | | |
| 5-methyltetrahydrofolate transport via anion | | −0.06 |
| exchange | | |
| folate transport via anion exchange | SLC19A1 | −0.06 |
| folate transport via anion exchange | | −0.06 |
| folate transport via anion exchange | | −0.06 |
| Facilitated diffusion | FOLR1; FOLR2 | −0.06 |
| H2O transport via diffusion | AQP1; AQP2; | −0.06 |
| | AQP4; AQP5; | |
| | AQP8; MIP | |
| 5-Oxoproline amidohydrolase (ATP- | OPLAH | −0.06 |
| hydrolysing) (ir) | | |
| CMP exchange | | −0.06 |
| Exchange of CTP(4-) | | −0.06 |
| Exchange of dTMP(2-) | | −0.06 |
| Exchange of dTTP(4-) | | −0.06 |
| Exchange of ITP(3-) | | −0.06 |
| CDP diphosphohydrolase Pyrimidine | ENTPD1; ENTPD3; | −0.06 |
| metabolism EC: 3.6.1.5 | ENTPD8 | |
| CTP diphosphohydrolase Pyrimidine | ENTPD1; ENTPD3; | −0.06 |
| metabolism EC: 3.6.1.5 | ENTPD8 | |
| ITP diphosphohydrolase Purine metabolism | ENTPD1; ENTPD3; | −0.06 |
| EC: 3.6.1.5 | ENTPD8 | |
| dTDP diphosphohydrolase Pyrimidine | ENTPD1; ENTPD3; | −0.06 |
| metabolism EC: 3.6.1.5 | ENTPD8 | |
| dTTP nucleotidohydrolase Pyrimidine | ENTPD1; ENTPD3; | −0.06 |
| metabolism EC: 3.6.1.39 | ENTPD8 | |
| 3-beta-hydroxysteroid-delta(8),delta(7)- | EBP | −0.06 |
| isomerase | | |
| lanosterol D24-reductase Biosynthesis of | DHCR24 | −0.06 |
| steroids EC: 1.3.1.72 | | |
| delta24-sterol reductase Biosynthesis of | DHCR24 | −0.06 |
| steroids EC: 1.3.1.72 | | |
| trans-Oct-2-enoyl-CoA reductase Fatty acid | MECR; PECR | −0.06 |
| elongation in mitochondria EC: 1.3.1.38 | | |
| trans-Dodec-2-enoyl-CoA reductase Fatty | MECR; PECR | −0.06 |
| acid elongation in mitochondria EC: 1.3.1.38 | | |
| trans-Tetradec-2-enoyl-CoA reductase Fatty | MECR; PECR | −0.06 |
| acid elongation in mitochondria EC: 1.3.1.38 | | |
| trans-Dec-2-enoyl-CoA reductase Fatty acid | MECR; PECR | −0.06 |
| elongation in mitochondria EC: 1.3.1.38 | | |
| trans-Hex-2-enoyl-CoA reductase Fatty acid | MECR; PECR | −0.06 |
| elongation in mitochondria EC: 1.3.1.38 | | |
| Na+/Proline-L symporter | ACE2; SLC38A1; | −0.06 |
| | SLC38A2; | |
| | SLC38A4; | |
| | SLC6A19; | |
| | TMEM27 | |
| ATPcytidine 5-phosphotransferase | UCK1; UCK2 | −0.06 |
| Nucleoside-diphosphate kinase (ATP: dUDP) | GM20390; NME2; | −0.06 |
| | NME3; NME6; | |
| | NME7 | |
| dUTP: cytidine 5-phosphotransferase | UCK1; UCK2 | −0.06 |
| Pyrimidine metabolism EC: 2.7.1.48 | | |
| dUTP: uridine 5-phosphotransferase | UCK1; UCK2; | −0.06 |
| Pyrimidine metabolism EC: 2.7.1.48 | UCKL1 | |
| uridine kinase (ATP: Uridine) | UCK1; UCK2; | −0.06 |
| | UCKL1 | |
| Hydroxymethylglutaryl-CoA reversible | | −0.06 |
| mitochondrial transport | | |
| fatty acid transport via diffusion | SLC27A2 | −0.06 |
| elaidic acid exchange | | −0.06 |
| fatty-acid-CoA ligase | ACSL1 | −0.06 |
| echange reaction for octadecanoate (n- | | −0.06 |
| C18: 0) | | |
| glucuronidated compound transport | | −0.06 |
| exchange reaction for blirubin mono- | | −0.06 |
| glucuronide | | |
| UDP-glucuronosyltransferase 1-10 | UGT1A8 | −0.06 |
| precursor, microsomal | | |
| RE0583 | | −0.06 |
| transport of 3aib_D into mitochondria | | −0.06 |
| D-3-amino-isobutyrate transport | | −0.06 |
| D-3-Amino-isobutanoate exchange | | −0.06 |
| Transport reaction | | −0.06 |
| Postulated transport reaction | | −0.06 |
| Very-long-chain-fatty-acid-CoA ligase | SLC27A2; | −0.06 |
| | SLC27A5 | |
| H transporter, peroxisome | | −0.06 |
| heparan sulfate proteoglycan exchange | | −0.06 |
| Exchange of L-iduronate | | −0.06 |
| beta-glucuronidase, lysosomal | GUSB | −0.06 |
| alpha-N-acetylglucosaminidase, lysosomal | NAGLU | −0.06 |
| alpha-N-acetylglucosaminidase, lysosomal | NAGLU | −0.06 |
| alpha-N-acetylglucosaminidase, lysosomal | NAGLU | −0.06 |
| alpha-N-acetylglucosaminidase, lysosomal | NAGLU | −0.06 |
| alpha-N-acetylglucosaminidase, lysosomal | NAGLU | −0.06 |
| heparan-N-sulfatase, lysosomal | SGSH | −0.06 |
| heparan-N-sulfatase, lysosomal | SGSH | −0.06 |
| heparan-N-sulfatase, lysosomal | SGSH | −0.06 |
| heparan-N-sulfatase, lysosomal | SGSH | −0.06 |
| heparan-glucosaminide N-acetyltransferase, | | −0.06 |
| lysosomal | | |
| heparan-glucosaminide N-acetyltransferase, | | −0.06 |
| lysosomal | | |
| heparan-glucosaminide N-acetyltransferase, | | −0.06 |
| lysosomal | | |
| heparan-glucosaminide N-acetyltransferase, | | −0.06 |
| lysosomal | | |
| heparan sulfate proteoglycan protease, | | −0.06 |
| lysosome (endosome) | | |
| heparan sulfate transport, extracellular to | | −0.06 |
| lysosome | | |
| alpha-L-iduronidase, lysosomal | IDUA | −0.06 |
| alpha-L-iduronidase, lysosomal | IDUA | −0.06 |
| alpha-L-iduronidase, lysosomal | IDUA | −0.06 |
| L-iduronate transport, extracellular | | −0.06 |
| Iduronate transport into lysososme | SLC17A5 | −0.06 |
| degradation of proteoglycan linkage region, | | −0.06 |
| lysosomal | | |
| iduronate-2-sulfatase, lysosomal | IDS | −0.06 |
| iduronate-2-sulfatase, lysosomal | IDS | −0.06 |
| N-acetylglucosamine-3-sulfatase, lysosomal | | −0.06 |
| N-acetylglucosamine-3-sulfatase, lysosomal | | −0.06 |
| N-acetylglucosamine-3-sulfatase, lysosomal | | −0.06 |
| N-acetylglucosamine-6-sulfatase, lysosomal | GNS | −0.06 |
| N-acetylglucosamine-6-sulfatase, lysosomal | GNS | −0.06 |
| N-acetylglucosamine-6-sulfatase, lysosomal | GNS | −0.06 |
| 5′-nucleotidase (GMP), extracellular | NT5C; NT5E | −0.06 |
| Major Facilitator(MFS) TCDB: 2.A.1.44.1 | SLC43A1 | −0.06 |
| Major Facilitator(MFS) TCDB: 2.A.1.44.1 | SLC43A1 | −0.06 |
| 5,6,7,8-Tetrahydrofolate exchange | | −0.06 |
| tetrahydrofolate transport via anion | SLC19A1 | −0.06 |
| exchange | | |
| RE3421 | | −0.06 |
| biotinidase (biotin), extracellular | BTD | −0.06 |
| Biocytin exchange | | −0.06 |
| Biotin exchange | | −0.06 |
| L-Lysine exchange | | −0.06 |
| production of hexadecanedioylcarnitine | CPT1A; CPT1B; | −0.06 |
| | CPT1C | |
| transport of Hexadecanedioic acid mono-L- | | −0.06 |
| carnitine ester into extra cellular space | | |
| exchange reaction for Hexadecanedioic acid | | −0.06 |
| mono-L-carnitine ester | | |
| fatty acid activation(C16DC)er | ACSL5 | −0.06 |
| fatty acid omega oxidation(C16-->w- | CYP4F15 | −0.06 |
| OHC16)er | | |
| fatty acid omega oxidation(w-OHC16-->C16DC)c | ADH5; ALDH3A2 | −0.06 |
| transport of hexadecanedioc acid by | | −0.06 |
| diffusion | | |
| transport of hexadecanedioyl coa | DBI | −0.06 |
| transport of w-hydroxypalmitic acid by | | −0.06 |
| diffusion | | |
| Y+LAT2 Utilized transport | SLC7A6 | −0.06 |
| alanine-glyoxylate transaminase | AGXT | −0.06 |
| (irreversible), (peroxisomal) | | |
| hydroxypyruate transport, peroxisomal | | −0.06 |
| L-serine transport, peroxisomal | | −0.06 |
| serine-pyruvate aminotransferase | AGXT | −0.06 |
| (irreversible), peroxisomal | | |
| Lactose exchange | | −0.06 |
| acetate-CoA ligase (ADP-forming) | ACSS2 | −0.06 |
| Methylglyoxal exchange | | −0.06 |
| Methylglyoxal transport (cytosol to | | −0.06 |
| extracellular) | | |
| CTP synthase (glutamine) | CTPS; CTPS2 | −0.06 |
| retinal dehydrogenase | | −0.06 |
| N-acetyllactosaminide beta-1,3-N- | B3GNT2; B3GNT3; | −0.06 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.06 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.06 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.06 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.06 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.06 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.06 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.06 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.06 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.06 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.06 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.06 |
| acetylglucosaminyltransferase, Golgi | B3GNT4; B3GNT7; | |
| apparatus | B3GNT8; | |
| | B3GNTL1; | |
| | B4GAT1 | |
| endo-beta-N-acetylglucosaminidase, | | −0.06 |
| lysosomal | | |
| alpha-fucosidase, lysosomal | FUCA1 | −0.06 |
| beta-N-acetylglucosaminylglycopeptide beta- | B4GALT1; | −0.06 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.06 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.06 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.06 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.06 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.06 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.06 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.06 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.06 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.06 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.06 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.06 |
| 1,4-galactosyltransferase, Golgi | B4GALT2; | |
| | B4GALT3; | |
| | B4GALT5 | |
| beta-galactosidase, lysosomal | CTSA; GALNS; | −0.06 |
| | GLB1; NEU1 | |
| beta-galactosidase, lysosomal | CTSA; GALNS; | −0.06 |
| | GLB1; NEU1 | |
| beta-galactosidase, lysosomal | CTSA; GALNS; | −0.06 |
| | GLB1; NEU1 | |
| beta-galactosidase, lysosomal | CTSA; GALNS; | −0.06 |
| | GLB1; NEU1 | |
| beta-galactosidase, lysosomal | CTSA; GALNS; | −0.06 |
| | GLB1; NEU1 | |
| beta-galactosidase, lysosomal | CTSA; GALNS; | −0.06 |
| | GLB1; NEU1 | |
| beta-galactosidase, lysosomal | CTSA; GALNS; | −0.06 |
| | GLB1; NEU1 | |
| beta-galactosidase, lysosomal | CTSA; GALNS; | −0.06 |
| | GLB1; NEU1 | |
| beta-galactosidase, lysosomal | CTSA; GALNS; | −0.06 |
| | GLB1; NEU1 | |
| beta-galactosidase, lysosomal | CTSA; GALNS; | −0.06 |
| | GLB1; NEU1 | |
| beta-galactosidase, lysosomal | CTSA; GALNS; | −0.06 |
| | GLB1; NEU1 | |
| beta-galactosidase, lysosomal | CTSA; GALNS; | −0.06 |
| | GLB1; NEU1 | |
| beta-galactosidase, lysosomal | CTSA; GALNS; | −0.06 |
| | GLB1; NEU1 | |
| glycosylasparaginase, lysosomal | AGA | −0.06 |
| keratan sulfate I transport, golgi to | | −0.06 |
| extracellular | | |
| keratan sulfate I transport, extracellular to | | −0.06 |
| lysosome | | |
| beta-N-acetylhexosaminidase, lysosomal | HEXA; HEXB | −0.06 |
| beta-N-acetylhexosaminidase, lysosomal | HEXA; HEXB | −0.06 |
| beta-N-acetylhexosaminidase, lysosomal | HEXA; HEXB | −0.06 |
| beta-N-acetylhexosaminidase, lysosomal | HEXA; HEXB | −0.06 |
| beta-N-acetylhexosaminidase, lysosomal | HEXA; HEXB | −0.06 |
| beta-N-acetylhexosaminidase, lysosomal | HEXA; HEXB | −0.06 |
| beta-N-acetylhexosaminidase, lysosomal | HEXA; HEXB | −0.06 |
| beta-N-acetylhexosaminidase, lysosomal | HEXA; HEXB | −0.06 |
| beta-N-acetylhexosaminidase, lysosomal | HEXA; HEXB | −0.06 |
| beta-N-acetylhexosaminidase, lysosomal | HEXA; HEXB | −0.06 |
| beta-N-acetylhexosaminidase, lysosomal | HEXA; HEXB | −0.06 |
| beta-N-acetylhexosaminidase, lysosomal | HEXA; HEXB | −0.06 |
| beta-N-acetylhexosaminidase, lysosomal | HEXA; HEXB | −0.06 |
| beta-N-acetylhexosaminidase A, lysosomal | HEXA; HEXB | −0.06 |
| beta-N-acetylhexosaminidase A, lysosomal | HEXA; HEXB | −0.06 |
| beta-N-acetylhexosaminidase A, lysosomal | HEXA; HEXB | −0.06 |
| beta-N-acetylhexosaminidase A, lysosomal | HEXA; HEXB | −0.06 |
| beta-N-acetylhexosaminidase A, lysosomal | HEXA; HEXB | −0.06 |
| beta-N-acetylhexosaminidase A, lysosomal | HEXA; HEXB | −0.06 |
| beta-N-acetylhexosaminidase A, lysosomal | HEXA; HEXB | −0.06 |
| beta-N-acetylhexosaminidase A, lysosomal | HEXA; HEXB | −0.06 |
| beta-N-acetylhexosaminidase A, lysosomal | HEXA; HEXB | −0.06 |
| beta-N-acetylhexosaminidase A, lysosomal | HEXA; HEXB | −0.06 |
| beta-N-acetylhexosaminidase A, lysosomal | HEXA; HEXB | −0.06 |
| beta-galactoside alpha-2,3-sialyltransferase | ST3GAL3 | −0.06 |
| (complex N-glycan) | | |
| galactose/N-acetylglucosamine 6-O- | CHST2; CHST4; | −0.06 |
| sulfotransferase, Golgi apparatus | CHST5 | |
| galactose/N-acetylglucosamine 6-O- | CHST2; CHST4; | −0.06 |
| sulfotransferase, Golgi apparatus | CHST5 | |
| galactose/N-acetylglucosamine 6-O- | CHST2; CHST4; | −0.06 |
| sulfotransferase, Golgi apparatus | CHST5 | |
| galactose/N-acetylglucosamine 6-O- | CHST2; CHST4; | −0.06 |
| sulfotransferase, Golgi apparatus | CHST5 | |
| galactose/N-acetylglucosamine 6-O- | CHST2; CHST4; | −0.06 |
| sulfotransferase, Golgi apparatus | CHST5 | |
| galactose/N-acetylglucosamine 6-O- | CHST1; CHST3 | −0.06 |
| sulfotransferase, Golgi apparatus | | |
| galactose/N-acetylglucosamine 6-O- | CHST2; CHST4; | −0.06 |
| sulfotransferase, Golgi apparatus | CHST5 | |
| galactose/N-acetylglucosamine 6-O- | CHST2; CHST4; | −0.06 |
| sulfotransferase, Golgi apparatus | CHST5 | |
| galactose/N-acetylglucosamine 6-O- | CHST2; CHST4; | −0.06 |
| sulfotransferase, Golgi apparatus | CHST5 | |
| galactose/N-acetylglucosamine 6-O- | CHST2; CHST4; | −0.06 |
| sulfotransferase, Golgi apparatus | CHST5 | |
| galactose/N-acetylglucosamine 6-O- | CHST2; CHST4; | −0.06 |
| sulfotransferase, Golgi apparatus | CHST5 | |
| galactose/N-acetylglucosamine 6-O- | CHST2; CHST4; | −0.06 |
| sulfotransferase, Golgi apparatus | CHST5 | |
| galactose-6-sulfate sulfatase, lysosomal | CTSA; GALNS; | −0.06 |
| | GLB1; NEU1 | |
| N-acetylglucosamine-6-sulfatase, lysosomal | GNS | −0.06 |
| N-acetylglucosamine-6-sulfatase, lysosomal | GNS | −0.06 |
| N-acetylglucosamine-6-sulfatase, lysosomal | GNS | −0.06 |
| N-acetylglucosamine-6-sulfatase, lysosomal | GNS | −0.06 |
| N-acetylglucosamine-6-sulfatase, lysosomal | GNS | −0.06 |
| N-acetylglucosamine-6-sulfatase, lysosomal | GNS | −0.06 |
| N-acetylglucosamine-6-sulfatase, lysosomal | GNS | −0.06 |
| N-acetylglucosamine-6-sulfatase, lysosomal | GNS | −0.06 |
| N-acetylglucosamine-6-sulfatase, lysosomal | GNS | −0.06 |
| N-acetylglucosamine-6-sulfatase, lysosomal | GNS | −0.06 |
| N-acetylglucosamine-6-sulfatase, lysosomal | GNS | −0.06 |
| sialidase, lysosomal | CTSA; GALNS; | −0.06 |
| | GLB1; NEU1 | |
| 5-Hydroxy-L-tryptophan decarboxy-lyase | DDC | −0.06 |
| Serotonin exchange | | −0.06 |
| adenylate kinase (d form) | AK5 | −0.06 |
| adenylate kinase (d form) | AK5 | −0.06 |
| ATP: AMP phosphotransferase Purine | AK1; AK2; AK4; | −0.06 |
| metabolism EC: 2.7.4.11 | AK5; AK7 | |
| ATP: AMP phosphotransferase Purine | AK1; AK2; AK4; | −0.06 |
| metabolism EC: 2.7.4.11 | AK5; AK7 | |
| exchange reaction for pectin-glycocholate | | −0.06 |
| complex | | |
| binding of pectins with glycocholate in the | | −0.06 |
| intestinal lumen, reducing serum cholesterol | | |
| levels. | | |
| Y+LAT2 Utilized transport | SLC7A6 | −0.06 |
| Y+LAT2 Utilized transport | SLC7A6 | −0.06 |
| Y+LAT2 Utilized transport | SLC7A6 | −0.06 |
| Isoleucine mitochondrial transport | | −0.07 |
| isoleucine transaminase, mitochondrial | BCAT2 | −0.07 |
| gamma-linolenic acid exchange | | −0.07 |
| fatty acid transport via diffusion | SLC27A5 | −0.07 |
| Glucuronate 1-phosphate phosphatase | | −0.07 |
| UDPglucuronate uridine- | | −0.07 |
| monophosphohydrolase | | |
| R total exchange | | −0.07 |
| Y+LAT2 Utilized transport | SLC7A6 | −0.07 |
| Y+LAT2 Utilized transport | SLC7A6 | −0.07 |
| Y+LAT2 Utilized transport | SLC7A6 | −0.07 |
| Utilized transport | | −0.07 |
| methenyltetrahydrifikate cyclohydrolase, | MTHFD1L; | −0.07 |
| mitochondrial | MTHFD2 | |
| RE3198 | | −0.07 |
| hypotaurine: NAD+ oxidoreductase Taurine | | −0.07 |
| and hypotaurine metabolism EC: 1.8.1.3 | | |
| RE2625 | | −0.07 |
| RE3251 | | −0.07 |
| RE3252 | | −0.07 |
| stearoyl-CoA desaturase (n-C18: 0CoA −> n- | SCD4 | −0.07 |
| C18: 1CoA) | | |
| octadecenoate (n-C18: 1) exchange | | −0.07 |
| lipid, flip-flop intracellular transport | | −0.07 |
| chenodeoxycholate exchange | | −0.07 |
| 5-beta-cytochrome P450, family 27, | CYP27A1 | −0.07 |
| subfamily A, polypeptide 1 | | |
| alcohol dehydrogenase Bile acid biosynthesis | ADH1; ADH4; | −0.07 |
| EC: 1.1.1.1 | ADH5; ADH6A; | |
| | ADH7 | |
| Postulated transport reaction | | −0.07 |
| RE1804 | | −0.07 |
| RE1804 | CYP27A1 | −0.07 |
| RE1807 | CYP27A1 | −0.07 |
| RE1810 | | −0.07 |
| RE1826 | | −0.07 |
| Very-long-chain-fatty-acid-CoA ligase | SLC27A2; | −0.07 |
| | SLC27A5 | |
| lipid, flip-flop intracellular transport | | −0.07 |
| N-acetylgalactosamine kinase | | −0.07 |
| N-acetylgalactosamine kinase (ITP) | | −0.07 |
| N-acetyl-galactosamine lysosomal efflux | | −0.07 |
| DM Ser-Gly/Ala-X-Gly(ly) | | −0.07 |
| D-Xylose exchange | | −0.07 |
| UDP-N-acetylgalactosamine diphosphorylase | | −0.07 |
| D-xylose reversible transport | SLC2A3 | −0.07 |
| Xylose efflux from lysosome | | −0.07 |
| L-isoleucine transport in via sodium symport | ACE2; SLC6A14; | −0.07 |
| | SLC6A19; | |
| | TMEM27 | |
| L-valine transport in via sodium symport | ACE2; SLC6A14; | −0.07 |
| | SLC6A19; | |
| | TMEM27 | |
| L-methionine transport in via sodium | ACE2; SLC22A5; | −0.07 |
| symport | SLC38A1; | |
| | SLC38A2; | |
| | SLC6A14; | |
| | SLC6A19; | |
| | TMEM27 | |
| glycine transport via sodium symport | ACE2; SLC38A1; | −0.07 |
| | SLC38A2; | |
| | SLC38A4; | |
| | SLC6A14; | |
| | SLC6A19; | |
| | TMEM27 | |
| Acetoacetate exchange | | −0.07 |
| leukotriene A4 exchange | | −0.07 |
| L-proline reversible transport via proton | SLC36A1; | −0.07 |
| symport | SLC36A2 | |
| Beta oxidation fatty acid | ACADM; ACADS | −0.07 |
| Postulated transport reaction | | −0.07 |
| phosphatidylinositol 5-kinase | PIP5K1A; | −0.07 |
| | PIP5K1B; | |
| | PIP5K1C | |
| degradation of proteoglycan linkage region, | | −0.07 |
| lysosomal | | |
| degradation of proteoglycan linkage region, | | −0.07 |
| lysosomal | | |
| beta-N-acetylhexosaminidase, lysosomal | HEXA; HEXB | −0.07 |
| N-acetylgalactosamine-4-sulfatase, | ARSB | −0.07 |
| lysosomal | | |
| CO2 endoplasmic reticular transport via | | −0.07 |
| diffusion | | |
| fatty-acyl-CoA elongation (n-C18: 3CoA) | ELOVL2; EL0VL4; | −0.07 |
| | ELOVL5; ELOVL6 | |
| O2 transport, endoplasmic reticulum | | −0.07 |
| EC: 2.3.1.86 | | −0.07 |
| EC: 6.2.1.3 | ACSBG2; ACSL1; | −0.07 |
| | ACSL3; ACSL4; | |
| | ACSL5; ACSL6; | |
| | SLC27A2 | |
| Utilized transport | | −0.07 |
| Utilized transport | | −0.07 |
| RE3241 | ELOVL1; ELOVL2; | −0.07 |
| | ELOVL5; ELOVL6 | |
| RE3242 | ELOVL1 | −0.07 |
| RE3243 | ELOVL1 | −0.07 |
| RE3244 | ELOVL1 | −0.07 |
| ethanolamine phosphotransferase | CEPT1 | −0.07 |
| phosphoethanolamine cytidyltransferase | PCYT2 | −0.07 |
| Phosphatidylserine synthase homo sapiens | PTDSS2 | −0.07 |
| acyl-CoA dehydrogenase (2-methylbutanoyl- | ACADM; ACADSB | −0.07 |
| CoA), mitochondrial | | |
| (S)-2-methylbutanoyl-CoA: acceptor 2,3- | ACADM; ACADS; | −0.07 |
| oxidoreductase Valine, leucine and isoleucine | ACADSB | |
| degradation EC: 1.3.99.12 | | |
| exchange reaction for L-alanine | | −0.07 |
| Thiamin triphosphate exchange | | −0.07 |
| Thiamine triphosphate transport in via anion | SLC19A1 | −0.07 |
| antiport | | |
| estradiol glucuronide transport via | SLCO1A1; | −0.07 |
| bicarbonate countertransport | SLCO1B2; | |
| | SLCO1C1; | |
| | SLCO4A1 | |
| glucuronidated compound transport | | −0.07 |
| estradiol intracellular transport | | −0.07 |
| ABC transporter | ABCC3 | −0.07 |
| glucuronidated compound transport | | −0.07 |
| estradiol glucuronide exchange | | −0.07 |
| estrone glucuronide exchange | | −0.07 |
| UDP-glucuronosyltransferase 1-10 | UGT1A8 | −0.07 |
| precursor, microsomal | | |
| UDP-glucuronosyltransferase 1-10 | UGT1A8 | −0.07 |
| precursor, microsomal | | |
| RE2660 | | −0.07 |
| fatty acid beta oxidation (C18: 4-->C16: 3)m | HADHA; HADHB | −0.07 |
| isomerization of (C18: 4)m | EC11 | −0.07 |
| fatty acid beta oxidation(C20: 4-->C18: 4)m | ACADVL; HADHA; | −0.07 |
| | HADHB | |
| fatty acid beta oxidation(C22: 5-->C20: 4)m | HADHA; HADHB | −0.07 |
| fatty acid beta oxidation(C22: 5-->C22: 6)m | ACADVL | −0.07 |
| isomerization(C22: 5)m | EC11 | −0.07 |
| fatty acid beta oxidation(C22: 6-->C22: 5)m | DECR1 | −0.07 |
| 2-Aminoacrylate hydrolysis | SDS | −0.07 |
| L-serine ammonia-lyase Glycine, serine and | SDS | −0.07 |
| threonine metabolism EC: 4.3.1.17 | | |
| L-Serine hydro-lyase | SDS | −0.07 |
| methenyltetrahydrofolate cyclohydrolase | MTHFD1; | −0.07 |
| | MTHFD2L | |
| fructose-bisphosphate aldolase | ALDOART2; | −0.07 |
| | ALDOB; ALDOC | |
| 2-Hydroxybutyrate: NAD+ oxidoreductase | LDHA; LDHAL6B; | −0.07 |
| | LDHB; LDHC; | |
| | UEVLD | |
| 2-Hydroxybutyrate exchange | | −0.07 |
| Transport of L-Isoleucine into the intestinal | SLC6A14 | −0.07 |
| cells by ATB0 transporter | | |
| transport of L-Methionine into the intestinal | SLC6A14 | −0.07 |
| cells by ATB0 transporter | | |
| transport of L-Valine into the intestinal cells | SLC6A14 | −0.07 |
| by ATB0 transporter | | |
| Proline dehydrogenase (m) | PRODH | −0.07 |
| Major Facilitator(MFS) TCDB: 2.A.1.44.1 | SLC43A1 | −0.07 |
| Major Facilitator(MFS) TCDB: 2.A.1.44.1 | SLC43A1 | −0.07 |
| Y+LAT2 Utilized transport | SLC7A6 | −0.07 |
| Y+LAT2 Utilized transport | SLC7A6 | −0.07 |
| ferroxidase | HEPH | −0.07 |
| 9-cis-retinol exchange | | −0.07 |
| cis-11-retinol exchange | | −0.07 |
| retinyl ester hydrolase (9-cis), extracellular | | −0.07 |
| retinyl ester hydrolase (11-cis), extracellular | | −0.07 |
| glucose-6-phosphate isomerase | GPI1 | −0.07 |
| transport of L-Cysteine into the cell coupled | SLC38A5 | −0.07 |
| with co-transport with Sodium and counter | | |
| transport with proton by SNAT5 transporter. | | |
| Transport of N-carbamoyl-L-aspartate | | −0.07 |
| dihydoorotic acid dehydrogenase | DHODH | −0.07 |
| (quinone10) | | |
| dihydroorotase | CAD | −0.07 |
| Exchange of N-carbamoyl-L-aspartate | | −0.07 |
| Exchange of orotate | | −0.07 |
| glucuronidated compound transport | ABCC1 | −0.07 |
| glucuronidated compound transport | | −0.07 |
| glucuronidated compound transport | ABCC1 | −0.07 |
| glucuronidated compound transport (ER) | | −0.07 |
| 5alpha-Dihydrotestosterone glucuronide | | −0.07 |
| exchange | | |
| androsterone glucuronide exchange | | −0.07 |
| testosterone glucuronide exchange | | −0.07 |
| glucuronidated compound transport | ABCC1 | −0.07 |
| glucuronidated compound transport | | −0.07 |
| UDP-glucuronosyltransferase 1-10 | UGT1A8; | −0.07 |
| precursor, microsomal | UGT2B34 | |
| UDP-glucuronosyltransferase 1-10 | UGT1A8; | −0.07 |
| precursor, microsomal | UGT2B34 | |
| UDP-glucuronosyltransferase 1-10 | UGT2B34 | −0.07 |
| precursor, microsomal | | |
| alanine racemase | PROSC | −0.07 |
| D-Alanine exchange | | −0.07 |
| 5alpha-Dihydrotestosterone sulfotransferase | SULT1A1; | −0.07 |
| | SULT2A1 | |
| Dehydroepiandrosterone sulfotransferase | SULT1A1; | −0.07 |
| | SULT1E1; | |
| | SULT2A1 | |
| Estrogen sulfotransferase | SULT1A1; | −0.07 |
| | SULT1E1 | |
| RE0908 | | −0.07 |
| RE0912 | SULT1A1; | −0.07 |
| | SULT4A1 | |
| RE3218 | | −0.07 |
| Steryl-sulfatase | | −0.07 |
| Steryl-sulfatase | | −0.07 |
| D-galactose transport via proton symport | SLC5A1 | −0.07 |
| 5beta-Cholestane-3alpha,7alpha,12alpha- | CYP27A1 | −0.07 |
| triol, NADPH: oxygen oxidoreductase (26- | | |
| hydroxylating) Bile acid biosynthesis | | |
| EC: 1.14.13.15 | | |
| Transport reaction | | −0.07 |
| Postulated transport reaction | | −0.07 |
| ATP-binding Cassette (ABC) | ABCD3 | −0.07 |
| TCDB: 3.A.1.203.1 | | |
| RE2625 | | −0.07 |
| Very-long-chain-fatty-acid-CoA ligase | SLC27A2 | −0.07 |
| Y+LAT2 Utilized transport | SLC7A6 | −0.07 |
| GTP: alpha-D-mannose-1-phosphate | GMPPA; GMPPB | −0.07 |
| guanylyltransferase Fructose and mannose | | |
| metabolism EC: 2.7.7.13 | | |
| Acetoacetate exchange | | −0.07 |
| catalase A, peroxisomal | CAT | −0.07 |
| adenylate kinase | AK1; AK2; AK5; | −0.07 |
| | AK7 | |
| adenylate kinase | AK1; AK2; AK5; | −0.07 |
| | AK7 | |
| adentylate kinase (GTP) | AK5 | −0.07 |
| adentylate kinase (GTP) | AK5 | −0.07 |
| B-alanine secretion via secretory vesicle | SLC32A1 | −0.07 |
| (ATP driven) | | |
| exchange reaction for L-serine | | −0.07 |
| glucose transport, Golgi apparatus | SLC2A1 | −0.07 |
| b-galactosidase, extracellular | GLB1; LCT | −0.07 |
| lactose transport from Golgi to extracellular | | −0.07 |
| (via vesicle) | | |
| UDP-Gal Golgi transport via CMP antiport | SLC35A2 | −0.07 |
| UDPgalactose: D-glucose 4-beta-D- | B4GALT1; | −0.07 |
| galactosyltransferase, Golgi apparatus | B4GALT2; LALBA | |
| nucleoside-diphosphatase (UDP), Golgi | ENTPD4 | −0.07 |
| apparatus | | |
| phosphate transport, Golgi apparatus | | −0.07 |
| V-type ATPase, H+ transporting, lysosomal | ATP6V0A1; | −0.07 |
| | ATP6V0A2; | |
| | ATP6V0A4; | |
| | ATP6V0B; | |
| | ATP6V0C; | |
| | ATP6V0D1; | |
| | ATP6V0D2; | |
| | ATP6V0E; | |
| | ATP6V1A; | |
| | ATP6V1B1; | |
| | ATP6V1B2; | |
| | ATP6V1C1; | |
| | ATP6V1C2; | |
| | ATP6V1D; | |
| | ATP6V1E1; | |
| | ATP6V1E2; | |
| | ATP6V1F; | |
| | ATP6V1G1; | |
| | ATP6V1G2; | |
| | ATP6V1G3; | |
| | ATP6V1H; TCIRG1 | |
| beta-glucuronidase, lysosomal | GUSB; HYAL3 | −0.07 |
| beta-glucuronidase, lysosomal | GUSB | −0.07 |
| glucuronate transport into lysososme | SLC17A5 | −0.07 |
| hyaluronan transport, extracellular to | | −0.07 |
| lysosome | | |
| beta-N-acetylhexosaminidase, lysosomal | HEXA; HEXB | −0.07 |
| Postulated transport reaction | | −0.07 |
| Transport reaction | | −0.07 |
| 5-formyltetrahydrofolate transport via anion | SLC19A1 | −0.07 |
| exchange | | |
| 5-Formyltetrahydrofolate exchange | | −0.07 |
| 5-formethyltetrahydrofolate cyclo-ligase | MTHFSL | −0.07 |
| thiamin pyrophosphatase | | −0.07 |
| EC: 6.2.1.3 | ACSBG2; ACSL1; | −0.07 |
| | ACSL3; ACSL4; | |
| | ACSL5; ACSL6; | |
| | SLC27A2 | |
| 2-Hydroxybutyrate: NAD+ oxidoreductase | LDHA; LDHAL6B; | −0.07 |
| | LDHB; LDHC; | |
| | UEVLD | |
| 2-Hydroxybutyrate exchange | | −0.07 |
| L-glutamine reversible transport via sodium | ACE2; SLC38A1; | −0.07 |
| symport | SLC38A2; | |
| | SLC38A4; | |
| | SLC6A14; | |
| | SLC6A19; | |
| | TMEM27 | |
| Fe(III) reduction (ascorbate) | CYBRD1 | −0.07 |
| Fe(II): oxygen oxidoreductase Porphyrin and | CP; FTH1; FTL1; | −0.07 |
| chlorophyll metabolism EC: 1.16.3.1 | FTMT | |
| FMNALKPIe | ACP1; ALPPL2 | −0.07 |
| Facilitated diffusion | | −0.07 |
| malate dehydrogenase | MDH1; MDH1B | −0.07 |
| Cytosine deaminase | | −0.07 |
| cytosine transport via facilated diffusion | SLC29A2 | −0.07 |
| Cytosine exchange | | −0.07 |
| catalase | CAT | −0.07 |
| phosphatidylinositol-3-phosphate 3- | | −0.07 |
| phosphatase, nuclear | | |
| RE1448 | | −0.07 |
| RE2973 | P1K3C2A; | −0.07 |
| | P1K3C2B; | |
| | P1K3C2G | |
| RE2974 | | −0.07 |
| phosphatidylinositol-5-phosphate 3-kinase | HCST; PIK3C2A; | −0.07 |
| | PIK3C2B; | |
| | PIK3C2G; PIK3C3; | |
| | PIK3CA; PIK3CB; | |
| | PIK3CD; PIK3CG; | |
| | PIK3R1; PIK3R2; | |
| | PIK3R3; PIK3R5 | |
| RE3269 | MTMR2; MTMR3 | −0.07 |
| dTTP demand | | −0.07 |
| DM dttp(n) | | −0.07 |
| RE0452 | | −0.07 |
| fatty-acyl-CoA synthase (n-C16: 0CoA) | ELOVL2; ELOVL5; | −0.07 |
| | ELOVL6; FASN | |
| 4-methylpentanal transport | | −0.07 |
| 4-methylpentanal transport (mitochondria) | | −0.07 |
| aldo-keto reductase family 1, member C1 | AKR1C6 | −0.07 |
| (chlordecone reductase; 3-alpha | | |
| hydroxysteroid dehydrogenase, type I; | | |
| dihydrodiol dehydrogenase 4) | | |
| hydroxyprogesterone transport | | −0.07 |
| 4-methylpentanal Exchange | | −0.07 |
| 20alpha-Hydroxyprogesterone exchange | | −0.07 |
| Progesterone exchange | | −0.07 |
| 3 beta-hydroxysteroid dehydrogenase/delta | HSD3B2 | −0.07 |
| 5-->4-isomerase type I | | |
| Cytochrome P450 11A1, mitochondrial | CYP11A1 | −0.07 |
| [Precursor] | | |
| pregnenolone intracellular transport | | −0.07 |
| Progesterone transport | | −0.07 |
| glycine N-choloyltransferase EC: 2.3.1.65 | BAAT | −0.07 |
| Hydroxymethylglutaryl CoA synthase (ir) | HMGCS2 | −0.07 |
| hydroxymethylglutaryl-CoA lyase | HMGCL; HMGCLL1 | −0.07 |
| 4-methyl-2- | BCKDHA; | −0.07 |
| oxopentanoate: [dihydrolipoyllysine-residue | BCKDHB; | |
| (2-methylpropanoyl)transferase] | TMEM91 | |
| lipoyllysine 2-oxidoreductase | | |
| (decarboxylating, acceptor-2- | | |
| methylpropanoylating) EC: 1.2.4.4 | | |
| 3-methylbutanoyl-CoA: enzyme N6- | DBT | −0.07 |
| (dihydrolipoyl)lysine S-(3- | | |
| methylbutanoyl)transferase Valine, leucine | | |
| and isoleucine degradation EC: 2.3.1.168 | | |
| 2-Deoxyadenosine 5-diphosphate: oxidized- | RRM1; RRM2; | −0.07 |
| thioredoxin 2-oxidoreductase Purine | RRM2B | |
| metabolism EC: 1.17.4.1 | | |
| nucleoside-diphosphatase (dUDP) | DTYMK | −0.07 |
| glucose 6-phosphate dehydrogenase | G6PD2 | −0.07 |
| Guanosine aminohydrolase EC: 3.5.4.15 | | −0.07 |
| Beta oxidation of long chain fatty acid | ACADM; ACADS | −0.07 |
| alpha-amylase, extracellular (strch1 −> | AMY2A5; SLC3A1; | −0.07 |
| strch2) | SLC3A2; SLC7A9 | |
| alpha-amylase, extracellular (glygn2 −> | AMY2A5; SLC3A1; | −0.07 |
| glygn4) | SLC3A2; SLC7A9 | |
| maltononaose exchange | | −0.07 |
| maltodecaose exchange | | −0.07 |
| glycogen, structure 2 (glycogenin-1,6-{7[1,4- | | −0.07 |
| Glc],4[1,4-Glc]}) exchange | | |
| exchange reaction for glycogen, structure 4 | | −0.07 |
| (glycogenin-1,6-{2[1,4-Glc],[1,4-Glc]}) | | |
| exchange reaction for glycogen, structure 5 | | −0.07 |
| (glycogenin-2[1,4-Glc]) | | |
| Exchange of Isomaltose | | −0.07 |
| maltoheptaose exchange | | −0.07 |
| Maltohexaose exchange | | −0.07 |
| starch, structure 1 (1,6-{7[1,4-Glc],4[1,4- | | −0.07 |
| Glc]}) exchange | | |
| exchange reaction for starch, structure 2 | | −0.07 |
| (1,6-(2[1,4-Glc],[1,4-Glc]}) | | |
| Tyr-194 of apo-glycogenin protein (primer | | −0.07 |
| for glycogen synthesis) exchange | | |
| glucoamylase, extracellular (glygn5 −> malt) | MGAM | −0.07 |
| oligo-1,6-glucosidase (glygn4 −> glygn5), | SIS | −0.07 |
| extracellular | | |
| oligo-1,6-glucosidase (strch2 −>strch3), | SIS | −0.07 |
| extracellular | | |
| Isomaltose 6-alpha-D-glucanohydrolase | SIS | −0.07 |
| Starch and sucrose metabolism EC: 3.2.1.10 | | |
| RE0915 | AMY2A5; LYZL1 | −0.07 |
| RE0926 | AMY2A5; LYZL1 | −0.07 |
| RE0935 | AMY2A5; LYZL1 | −0.07 |
| RE0944 | AMY2A5; LYZL1 | −0.07 |
| RE0958 | AMY2A5; LYZL1 | −0.07 |
| Y+LAT2 Utilized transport | SLC7A6 | −0.07 |
| 2-Oxoglutarate exchange | | −0.07 |
| 25-Hydroxyvitamin D3 exchange | | −0.07 |
| exchange reaction for calctriol | | −0.07 |
| RE1303 | | −0.07 |
| Reduced glutathione exchange | | −0.07 |
| R total 3 position exchange | | −0.08 |
| triacylglycerol (homo sapiens) exchange | | −0.08 |
| folate transport via anion exchange | SLC19A1 | −0.08 |
| H2O transport via diffusion | AQP1; AQP2; | −0.08 |
| | AQP4; AQP5; | |
| | AQP8; MIP | |
| Chenodeoxyglycocholate exchange | | −0.08 |
| taurochenodeoxycholate exchange | | −0.08 |
| malic enzyme (NADP) | MEI | −0.08 |
| D-Lactaldehyde: NAD+ oxidoreductase | ADH5 | −0.08 |
| (glutathione-formylating) | | |
| L-cysteine reversible transport via sodium | ACE2; SLC38A2; | −0.08 |
| symport | SLC6A14; | |
| | SLC6A19; | |
| | TMEM27 | |
| RE1518 | ACADL; ACOX1 | −0.08 |
| RE1519 | ACADL; ACOX1 | −0.08 |
| RE2998 | ACOX1 | −0.08 |
| RE3626 | ACOX1 | −0.08 |
| adenylosuccinate lyase | ADSL | −0.08 |
| adenylosuccinate synthase | ADSS; ADSSL1 | −0.08 |
| chondroitin sulfate D proteoglycan protease, | | −0.08 |
| lysosome (endosome) | | |
| chondroitin sulfate E proteoglycan protease, | | −0.08 |
| lysosome (endosome) | | |
| chondroitin sulfate D transport, extracellular | | −0.08 |
| to lysosome | | |
| chondroitin sulfate E transport, extracellular | | −0.08 |
| to lysosome | | |
| chondroitin sulfate D (GlcNAc6S-GlcA2S) | | −0.08 |
| proteoglycan exchange | | |
| chondroitin sulfate E (GalNAc4,6diS-GlcA) | | −0.08 |
| proteoglycan exchange | | |
| beta-glucuronidase, lysosomal | GUSB | −0.08 |
| beta-glucuronidase, lysosomal | GUSB | −0.08 |
| degradation of proteoglycan linkage region, | | −0.08 |
| lysosomal | | |
| beta-N-acetylhexosaminidase, lysosomal | HEXA; HEXB | −0.08 |
| beta-N-acetylhexosaminidase, lysosomal | HEXA; HEXB | −0.08 |
| beta-N-acetylhexosaminidase, lysosomal | HEXA; HEXB | −0.08 |
| beta-N-acetylhexosaminidase, lysosomal | HEXA; HEXB | −0.08 |
| beta-N-acetylhexosaminidase A, lysosomal | HEXA; HEXB | −0.08 |
| beta-N-acetylhexosaminidase A, lysosomal | HEXA; HEXB | −0.08 |
| beta-N-acetylhexosaminidase A, lysosomal | HEXA; HEXB | −0.08 |
| beta-N-acetylhexosaminidase A, lysosomal | HEXA; HEXB | −0.08 |
| glucuronate-2-sulfatase, lysosomal | | −0.08 |
| glucuronate-2-sulfatase, lysosomal | | −0.08 |
| N-acetylgalactosamine-4-sulfatase, | ARSB | −0.08 |
| lysosomal | | |
| N-acetylgalactosamine-4-sulfatase, | ARSB | −0.08 |
| lysosomal | | |
| N-acetylgalactosamine-6-sulfatase, | CTSA; GALNS; | −0.08 |
| lysosomal | GLB1; NEU1 | |
| N-acetylgalactosamine-6-sulfatase, | CTSA; GALNS; | −0.08 |
| lysosomal | GLB1; NEU1 | |
| N-acetylgalactosamine-6-sulfatase, | CTSA; GALNS; | −0.08 |
| lysosomal | GLB1; NEU1 | |
| N-acetylgalactosamine-6-sulfatase, | CTSA; GALNS; | −0.08 |
| lysosomal | GLB1; NEU1 | |
| Y+LAT2 Utilized transport | SLC7A6 | −0.08 |
| Acetoacetate: CoA ligase (AMP-forming) | AACS | −0.08 |
| Butanoate metabolism EC: 6.2.1.16 | | |
| RE3011 | | −0.08 |
| RE3014 | CYP4F15; | −0.08 |
| | CYP4F39 | |
| RE3017 | | −0.08 |
| ornithine carbamoyltransferase, irreversible | OTC | −0.08 |
| Carbon-dioxide: ammonia ligase (ADP- | CPS1 | −0.08 |
| forming, carbamate-phosphorylating) Urea | | |
| cycle and metabolism of amino groups/ | | |
| Nitrogen metabolism EC: 6.3.4.16 | | |
| 7-alpha,27-Dihydroxycholesterol exchange | | −0.08 |
| oxysterol 7alpha-hydroxylase | CYP7B1 | −0.08 |
| cholesterol monooxygenase | | −0.08 |
| RE1796 | HSD3B2 | −0.08 |
| 27 trihydroxy cholesterol transport | | −0.08 |
| 27 trihydroxy cholesterol transport | | −0.08 |
| 3-Aminopropanoate: 2-oxoglutarate | ABAT | −0.08 |
| aminotransferase (m) | | |
| Beta-alanine reversible mitochondrial | | −0.08 |
| transport (diffusion) | | |
| phosphatidylinositol 3-phosphate 5-kinase | PIP5K1A; | −0.08 |
| | PIP5K1B; | |
| | PIP5K1C | |
| RE2973 | PIK3C2A; | −0.08 |
| | PIK3C2B; | |
| | PIK3C2G; PIK3C3; | |
| | PIK3CA; PIK3CB; | |
| | PIK3CG | |
| RE2974 | PIP4K2A; | −0.08 |
| | PIP4K2B; | |
| | PIP4K2C; | |
| | PIP5K1A; | |
| | PIP5K1B; | |
| | PIP5K1C | |
| retinoyl glucuronide exchange | | −0.08 |
| retinoyl glucuronide efflux | | −0.08 |
| retinoyl glucuronide efflux (13-cis) from ER | | −0.08 |
| retinoyl glucuronide efflux from ER | | −0.08 |
| retinoic acid transport in ER | | −0.08 |
| retinoic acid transport in ER (13-cis) | | −0.08 |
| UDP-glucuronosyltransferase 1-10 | UGT1A8 | −0.08 |
| precursor, microsomal | | |
| UDP-glucuronosyltransferase 1-10 | UGT1A1; UGT1A2; | −0.08 |
| precursor, microsomal (13-cis) | UGT1A7C; | |
| | UGT1A8; | |
| | UGT2B34 | |
| Prostaglandin F2alpha exchange | | −0.08 |
| beta-Carotene dioxygenase | BCO1 | −0.08 |
| beta-carotene transport via diffusion | | −0.08 |
| beta-Carotene exchange | | −0.08 |
| RE2799 | | −0.08 |
| 5 alpha dihydrotesterone transport | | −0.08 |
| 5 alpha dihydrotesterone intracellular | | −0.08 |
| transport | | |
| androsterone transport | | −0.08 |
| androsterone intracellular transport | | −0.08 |
| 5alpha-Dihydrotestosterone exchange | | −0.08 |
| Androsterone exchange | | −0.08 |
| 3-oxo-5-alpha-steroid 4-dehydrogenase | SRD5A1; SRD5A2 | −0.08 |
| 3-oxo-5-alpha-steroid 4-dehydrogenase | SRD5A1; SRD5A2 | −0.08 |
| thymidine transport (1:2 Na/Thymd | SLC28A3 | −0.08 |
| cotransport) | | |
| Transport of L-Histidine by y+ transporter | SLC7A1 | −0.08 |
| Thymine exchange | | −0.08 |
| thymine reversible transport via facilated | SLC29A2 | −0.08 |
| diffusion | | |
| exchange reaction for D-Sorbitol | | −0.08 |
| D-sorbitol transport, extracellular | | −0.08 |
| alcohol dehydrogenase (methanol) | ADH1; ADH4; | −0.08 |
| | ADH5; ADH6A; | |
| | ADH7; ADHFE1; | |
| | ZADH2 | |
| H2O transport, lysosomal | | −0.08 |
| Peroxidase (multiple substrates) | EPX; PRDX6 | −0.08 |
| stearoyl-CoA desaturase (n-C18: 0CoA −> n- | SCD4 | −0.08 |
| C18: 1CoA) | | |
| RE0583 | | −0.08 |
| Aminobutyraldehyde dehydrogenase | ALDH9A1 | −0.08 |
| Putrescine: oxygen oxidoreductase | AOC1; AOC2; | −0.08 |
| (deaminating) | AOC3 | |
| myo-inositol 1-phosphatase | IMPA1; IMPA2; | −0.08 |
| | MTMR1; MTMR2 | |
| phosphatidylinositol phospholipase C | PLCB1; PLCB2; | −0.08 |
| | PLCB3; PLCB4; | |
| | PLCD1; PLCD3; | |
| | PLCD4; PLCE1; | |
| | PLCG1; PLCG2; | |
| | PLCHI; PLCH2; | |
| | PLCL1; PLCXD2; | |
| | PLCZ1 | |
| RE3273 | PLD2 | −0.08 |
| cytidine deaminase | AICDA; CDA | −0.08 |
| citrate synthase | CSL | −0.08 |
| Diacylglycerol phosphate kinase | DGKA; DGKB; | −0.08 |
| (homo sapiens) | DGKD; DGKE; | |
| | DGKG; DGKH; | |
| | DGKI; DGKQ; | |
| | DGKZ | |
| phosphatidic acid phosphatase | PLPP1; PLPP2; | −0.08 |
| | PLPP3 | |
| Citrate oxaloacetate-lyase ((pro-3S)- | | −0.08 |
| CH2COO— -->acetate) Citrate cycle (TCA | | |
| cycle} EC: 4.1.3.6 | | |
| RE2649 | ACOT2; ACOT6; | −0.08 |
| | BAAT | |
| OROTGLUt | SLC22A7 | −0.08 |
| Postulated transport reaction | | −0.08 |
| D-Fructose 1-phosphate D-glyceraldehyde-3- | ALDOART2; | −0.08 |
| phosphate-lyase | ALDOB; ALDOC | |
| ketohexokinase (D-xylulose) | KHK | −0.08 |
| Dehydroepiandrosterone transport | | −0.08 |
| Dehydroepiandrosterone sulfate exchange | | −0.08 |
| testosterone sulfate exchange | | −0.08 |
| sulfonated testosterone transport | | −0.08 |
| testosterone sulfotransferase | SULT1A1; | −0.08 |
| | SULT2A1 | |
| Lysine mitochondrial transport via ornithine | SLC25A2 | −0.08 |
| carrier | | |
| arginine mitochondrial transport via | SLC25A2 | −0.08 |
| ornithine carrier | | |
| Lysine mitochondrial transport via ornithine | SLC25A2 | −0.08 |
| carrier | | |
| ornithine mitochondrial transport exchange | SLC25A2 | −0.08 |
| with citruline | | |
| Mitochondrial Carrier (MC) TCDB: 2.A.29.19.1 | SLC25A2 | −0.08 |
| Mitochondrial Carrier (MC) TCDB: 2.A.29.19.1 | SLC25A2 | −0.08 |
| ornithine mitochondrial transport via proton | SLC25A2 | −0.08 |
| antiport | | |
| ornithine mitochondrial transport via proton | SLC25A2 | −0.08 |
| antiport | | |
| ornithine mitochondrial transport exchange | SLC25A2 | −0.08 |
| with citruline | | |
| citrulline mitochondrial transport via proton | SLC25A2 | −0.08 |
| antiport | | |
| arginine mitochondrial transport via | SLC25A2 | −0.08 |
| ornithine carrier | | |
| citrulline mitochondrial transport via proton | SLC25A2 | −0.08 |
| antiport | | |
| glutamate-5-semialdehyde dehydrogenase | ALDH18A1 | −0.08 |
| (m) | | |
| glutamate 5-kinase (m) | ALDH18A1 | −0.08 |
| L-Glutamate 5-semialdehyde: NAD+ | ALDH4A1 | −0.08 |
| oxidoreductase Arginine and proline | | |
| metabolism EC: 1.5.1.12 | | |
| 5,6-Dihydrothymine: NAD+ oxidoreductase | | −0.08 |
| Pyrimidine metabolism EC: 1.3.1.1 | | |
| Postulated transport reaction | | −0.08 |
| Facilitated diffusion | | −0.08 |
| L-serine via sodium symport | ACE2; SLC38A1; | −0.08 |
| | SLC38A2; | |
| | SLC38A4; | |
| | SLC6A14; | |
| | SLC6A19; | |
| | TMEM27 | |
| L-threonine via sodium symport | ACE2; SLC38A1; | −0.08 |
| | SLC38A2; | |
| | SLC6A14; | |
| | SLC6A19; | |
| | TMEM27 | |
| dTTP transport via dUDP antiport | SLC25A19 | −0.08 |
| dTTP transport via dADP antiport | SLC25A19 | −0.08 |
| dTTP transport via dCDP antiport | SLC25A19 | −0.08 |
| dTTP transport via dTDP antiport | SLC25A19 | −0.08 |
| dTTP transport via dGDP antiport | SLC25A19 | −0.08 |
| RE0453 | NME4 | −0.08 |
| dTTP transport via ATP antiport | SLC25A19 | −0.08 |
| dTTP transport via ADP antiport | SLC25A19 | −0.08 |
| inositol-1,4-bisphosphate 1-phosphatase | INPP1 | −0.08 |
| myo-inositol 4-phosphatase | IMPA1; IMPA2 | −0.08 |
| phosphatidylinositol 4-phosphate | PLCB1; PLCB2; | −0.08 |
| phospholipase C | PLCB3; PLCB4; | |
| | PLCD1; PLCD3; | |
| | PLCD4; PLCE1; | |
| | PLCG1; PLCG2; | |
| | PLCHI; PLCH2; | |
| | PLCL1; PLCXD2; | |
| | PLCZ1 | |
| Nucleoside-diphosphate kinase (ATP: dADP) | GM20390; NME2; | −0.08 |
| | NME3; NME6; | |
| | NME7 | |
| 2-Deoxyadenosine 5-diphosphate: oxidized- | RRM1; RRM2; | −0.08 |
| thioredoxin 2-oxidoreductase Purine | RRM2B | |
| metabolism EC: 1.17.4.1 | | |
| 2-Deoxyadenosine 5-diphosphate: oxidized- | RRM1; RRM2; | −0.08 |
| thioredoxin 2-oxidoreductase Purine | RRM2B | |
| metabolism EC: 1.17.4.1 | | |
| 2-Deoxyadenosine 5-diphosphate: oxidized- | RRM1; RRM2; | −0.08 |
| thioredoxin 2-oxidoreductase Purine | RRM2B | |
| metabolism EC: 1.17.4.1 | | |
| 2-Deoxyadenosine 5-diphosphate: oxidized- | RRM1; RRM2; | −0.08 |
| thioredoxin 2-oxidoreductase Purine | RRM2B | |
| metabolism EC: 1.17.4.1 | | |
| ribonucleoside-diphosphate reductase (ADP) | RRM1; RRM2; | −0.08 |
| | RRM2B | |
| ribonucleoside-diphosphate reductase (GDP) | RRM1; RRM2; | −0.08 |
| | RRM2B | |
| ribonucleoside-diphosphate reductase (CDP) | RRM1; RRM2; | −0.08 |
| | RRM2B | |
| ribonucleoside-diphosphate reductase (UDP) | RRM1; RRM2; | −0.08 |
| | RRM2B | |
| Nucleoside-diphosphate kinase (ATP: dUDP) | GM20390; NME2; | −0.08 |
| | NME3; NME6; | |
| | NME7 | |
| ATP-binding Cassette (ABC) | ABCC4 | −0.08 |
| TCDB: 3.A.1.208.7 | | |
| Acetyl adenylate: CoA ligase (AMP-forming) | ACSS1; ACSS2 | −0.08 |
| Pyruvate metabolism EC: 6.2.1.1 | | |
| Acetate: CoA ligase (AMP-forming) Pyruvate | ACSS1; ACSS2 | −0.08 |
| metabolism EC: 6.2.1.1 | | |
| L-glutamine transport via electroneutral | | −0.08 |
| transporter | | |
| glutaminase (mitochondrial) | GLS; GLS2 | −0.08 |
| glycine reversible transport via proton | SLC36A1; | −0.08 |
| symport | SLC36A2 | |
| Transport of anthranilate | | −0.08 |
| Exchange of anthranilate | | −0.08 |
| kynureninase | KYNU | −0.08 |
| L-Formylkynurenine hydrolase | KYNU | −0.08 |
| N-Formylanthranilate amidohydrolase | AFMID | −0.08 |
| Tryptophan metabolism EC: 3.5.1.9 | | |
| aspartate N-acetyltransferase, mitochondrial | | −0.08 |
| N-Acetyl-L-aspartate amidohydrolase | ACY3; | −0.08 |
| | ASPA | |
| N-acetyl-L-aspartate transport | | −0.08 |
| (mitochondria to cytosol) | | |
| Facilitated diffusion | | −0.08 |
| thymd transport | SLC29A1 | −0.08 |
| Transport of q10 | | −0.08 |
| exchange reaction for ubiquinone | | −0.08 |
| exchange reaction for ubiquinol | | −0.08 |
| transport of ubiquinol into lymph | | −0.08 |
| exchange reaction for ptth | | −0.08 |
| PAN4PPe | | −0.08 |
| Estrone 3-sulfate exchange | | −0.08 |
| xenobiotic transport | | −0.08 |
| xenobiotic transport | | −0.08 |
| xenobiotic transport | | −0.08 |
| xenobiotic transport | | −0.08 |
| ebastine exchange | | −0.08 |
| hydroxylated ebastine exchange | | −0.08 |
| cytochrome p450 4F12/4F2 | CYP4F15 | −0.08 |
| 2-keto-4-methylthiobutyrate transamination | | −0.08 |
| L-2-aminoadipate shuttle | SLC25A21 | −0.08 |
| (cytosol/mitochondria) | | |
| 2-oxoadipate shuttle (cytosol/mitochondria) | SLC25A21 | −0.08 |
| 2-aminoadipate transaminase, irreversible | AADAT | −0.08 |
| L-2-Aminoadipate: 2-oxoglutarate | AADAT | −0.08 |
| aminotransferase Lysine biosynthesis/ | | |
| Lysine degradation EC: 2.6.1.39 | | |
| Retinol exchange | | −0.08 |
| Retinoate exchange | | −0.08 |
| retinal dehydrogenase | | −0.08 |
| retinal dehydrogenase (NADPH) | | −0.08 |
| Amino Acid-Polyamine-Organocation (APC) | SLC7A5 | −0.08 |
| TCDB: 2.A.3.8.1 | | |
| RE2973 | PIK3C2A; | −0.08 |
| | PIK3C2B; | |
| | PIK3C2G; PIK3C3; | |
| | PIK3CA; PIK3CB; | |
| | PIK3CG | |
| RE2974 | PIP4K2A; | −0.08 |
| | PIP4K2B; | |
| | PIP4K2C; | |
| | PIP5K1A; | |
| | PIP5K1B; | |
| | PIP5K1C | |
| RE3270 | | −0.08 |
| 4-aminobutanoate mitochondrial transport | | −0.08 |
| via diffusion | | |
| 4-aminobutyrate transaminase, reversible | ABAT | −0.08 |
| (mitochondrial) | | |
| Succinate-semialdehyde: NAD+ | ALDH5A1 | −0.08 |
| oxidoreductase Glutamate metabolism/ | | |
| Tyrosine metabolism/Butanoate | | |
| metabolism EC: 1.2.1.24 EC: 1.2.1.16 | | |
| nucleoside-diphosphate kinase (ATP: dTDP), | NME4 | −0.08 |
| mitochondrial | | |
| carnitine O-palmitoyltransferase | CPT1A; CPT1B; | −0.08 |
| | CPT1C | |
| carnitine transferase | CPT2 | −0.08 |
| transport into the mitochondria (carnitine) | SLC25A20 | −0.08 |
| Beta oxidation of fatty acid | ACADM; ACADS | −0.08 |
| Beta oxidation of long chain fatty acid | ACADM; ACADS | −0.08 |
| keratan sulfate II (core 2-linked) exchange | | −0.08 |
| keratan sulfate II (core 4-linked) exchange | | −0.08 |
| 1 acyl phosphoglycerol exchange | | −0.08 |
| phospholipase | PLA2G3 | −0.08 |
| betaine-homocysteine S-methyltransferase | BHMT; BHMT2 | −0.08 |
| dimethylglycine dehydrogenase, | DMGDH | −0.08 |
| mitochondrial | | |
| dimethylglycine transport via diffusion | | −0.08 |
| (cytosol to mitochondria) | | |
| formaldehyde transport via diffusion | | −0.08 |
| (mitochondrial) | | |
| Propionate exchange | | −0.08 |
| Beta oxidation of fatty acid | ACADM; ACADS | −0.08 |
| alanyl aminopeptidase (cys-gly) (e) | ANPEP | −0.08 |
| Free diffusion | | −0.08 |
| Isopentenyl diphosphate transport | | −0.08 |
| (peroxisome) | | |
| coenzyme A transport, nuclear | | −0.08 |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| Amino Acid-Polyamine-Organocation (APC) | SLC7A5 | −0.08 |
| TCDB: 2.A.3.8.1 | | |
| transport of docosanedioic acid by diffusion | | −0.08 |
| fatty acid omega oxidation by | CYP4F15 | −0.08 |
| hydroxylases(C22-->C22DC)er | | |
| N-acetylneuraminate transport into | SLC17A5 | −0.08 |
| lysososme | | |
| beta-N-acetylhexosaminidase, lysosomal | HEXA; HEXB | −0.08 |
| endo-beta-N-acetylglucosaminidase, | | −0.08 |
| lysosomal | | |
| L-Fucose exchange | | −0.08 |
| fucose-1-phosphate guanylyltransferase | FPGT; TNNI3K | −0.08 |
| glycoprotein 6-alpha-L-fucosyltransferase | FUT8 | −0.08 |
| Fucokinase | FUK | −0.08 |
| alpha-fucosidase, extracellular | FUCA2 | −0.08 |
| alpha-fucosidase, lysosomal | FUCA1 | −0.08 |
| L-fucose efflux from lysosome | | −0.08 |
| beta-N-acetylglucosaminylglycopeptide beta- | B4GALT1; | −0.08 |
| 1,4-galactosyltransferase | B4GALT2; | |
| | B4GALT3; | |
| | B4GALT5 | |
| beta-galactosidase, lysosomal | CTSA; GALNS; | −0.08 |
| | GLB1; NEU1 | |
| glycosylasparaginase, lysosomal | AGA | −0.08 |
| beta-galactoside alpha-2,6-sialyltransferase | ST6GAL1 | −0.08 |
| s2l2fn2m2masn transport, Golgi to | | −0.08 |
| extracellular | | |
| s2l2fn2m2masn transport, extracellular to | | −0.08 |
| lysosome | | |
| s2l2n2m2masn transport, extracellular to | | −0.08 |
| lysosome | | |
| sialidase, lysosomal | CTSA; GALNS; | −0.08 |
| | GLB1; NEU1 | |
| Cys-Gly exchange | | −0.08 |
| pyrroline-5-carboxylate reductase | PYCR1; PYCR2; | −0.08 |
| | PYCRL | |
| L-ProlineNAD+ 5-oxidoreductase | PRODH2 | −0.08 |
| Proline dehydrogenase | PRODH2 | −0.08 |
| RE3347 | | −0.08 |
| H2O transport, peroxisomal | | −0.09 |
| ubiquinol-6 cytochrome c reductase, | CYC1; MT-CYTB; | −0.09 |
| Complex III | UQCR10; UQCR11; | |
| | UQCRB; UQCRC1; | |
| | UQCRC2; | |
| | UQCRFS1; UQCRH; | |
| | UQCRQ | |
| L-Ascorbate exchange | | −0.09 |
| exchange reaction for dehydroascorbide(1-) | | −0.09 |
| Lecithin retinol acyltransferase | LRAT | −0.09 |
| Lecithin retinol acyltransferase (11-cis) | | −0.09 |
| Lecithin retinol acyltransferase (9-cis) | | −0.09 |
| retinyl ester hydrolase (9-cis) | | −0.09 |
| retinyl ester hydrolase (11-cis) | | −0.09 |
| Pyrophasphatase (dephospho-CoA, | | −0.09 |
| extracellular) | | |
| AMP exchange | | −0.09 |
| exchange reaction for dpcoa | | −0.09 |
| exchange reaction for pan4p | | −0.09 |
| 6-phosphogluconolactonase | PGLS | −0.09 |
| CO2 exchange | | −0.09 |
| Major Facilitator(MFS) TCDB: 2.A.18.8.1 | SLC36A1 | −0.09 |
| FAD diphosphatase | ENPP1 | −0.09 |
| D-glucose transport in via proton symport | SLC5A1 | −0.09 |
| transport of succinyl carnitine into the extra | | −0.09 |
| cellular space | | |
| exchange reaction for succinyl carnitine | | −0.09 |
| CO2 transporter via diffusion | | −0.09 |
| carboxylic acid dissociation | CAR1; CAR12; | −0.09 |
| | CAR13; CAR14; | |
| | CAR2; CAR3; | |
| | CAR4; CAR6; | |
| | CAR7; CAR9 | |
| Carbonic acid hydro-lyase Nitrogen | CAR1; CAR12; | −0.09 |
| metabolism EC: 4.2.1.1 | CAR13; CAR14; | |
| | CAR2; CAR4; | |
| | CAR6; CAR7; | |
| | CAR8; CAR9 | |
| glutaryl-CoA dehydrogenase (mitochondria) | GCDH | −0.09 |
| Glutaryl-CoA: (acceptor) 2,3-oxidoreductase | GCDH | −0.09 |
| (decarboxylating) Fatty acid metabolism | | |
| EC: 1.3.99.7 | | |
| formyltetrahydro folate dehydrogenase | ALDH1L1; | −0.09 |
| | ALDH1L2 | |
| transport of dTTP into mitochondria | | −0.09 |
| acetyl-CoA C-acetyltransferase, | ACAA2; ACAT1; | −0.09 |
| mitochondrial | HADHB | |
| 2-Methylprop-2-enoyl-CoA (2-Methylbut-2- | ECHS1; HADHA; | −0.09 |
| enoyl-CoA), mitochondrial | HADHB | |
| 3-hydroxyacyl-CoA dehydrogenase (2- | EHHADH; HADH; | −0.09 |
| Methylacetoacetyl-CoA), mitochondrial | HSD17B10 | |
| transport of L-Asparagine into the intestinal | SLC6A14 | −0.09 |
| cells by ATBO transporter | | |
| transport of L-Proline by the apical IMINO | SLC6A20B; | −0.09 |
| amino acid transporters in kidney and | TMEM27 | |
| intestine | | |
| Propanoyl-CoA: FAD 2,3-oxidoreductase, | ACAD11; ACAD12; | −0.09 |
| mitochondrial | ACAD8; ACAD9; | |
| | ACADM; ACADS; | |
| | ACADSB | |
| Propenoyl-CoA hydrolase (m) | AUH; ECHS1; | −0.09 |
| | EHHADH; HADHA; | |
| | HADHB | |
| 3-hydroxypropanoate: NAD+ oxidoreductase | | −0.09 |
| beta-Alanine metabolism/Propanoate | | |
| metabolism EC: 1.1.1.59 | | |
| 3-hydroxyisobutyryl-CoA hydrolase beta- | HIBCH | −0.09 |
| Alanine metabolism/Propanoate | | |
| metabolism EC: 3.1.2.4 | | |
| aldo-keto reductase family 1, member D1 | AKR1D1 | −0.09 |
| (delta 4-3-ketosteroid-5-beta-reductase) | | |
| 5-beta-cholestane-3-alpha,7-alpha,12-alpha- | CYP27A1 | −0.09 |
| triol 27-hydroxylase | | |
| sterol 12-alpha-hydroxylase | CYP8B1 | −0.09 |
| sterol 12-alpha-hydroxylase (nadh) | CYP8B1 | −0.09 |
| acyl-Coenzyme A dehydrogenase family, | ACAD9 | −0.09 |
| member 9 Bile acid biosynthesis EC: 1.3.3.6 | | |
| hydroxysteroid (17-beta) dehydrogenase 4 | HSD17B4 | −0.09 |
| Bile acid biosynthesis EC: 1.1.1.35 | | |
| (24R,25R)-3alpha,7alpha,12alpha,24- | HSD17B4 | −0.09 |
| tetrahydroxy-5beta-cholestanoyl- CoA | | |
| hydro-lyase Bile acid biosynthesis | | |
| EC: 4.2.1.107 | | |
| 3alpha,7alpha,12alpha-Trihydroxy-5beta- | AKR1C6 | −0.09 |
| cholestane: NADP+ oxidoreductase (B- | | |
| specific); 3alpha,7alpha,12alpha-Trihydroxy- | | |
| 5beta-cholestane: NADP+ oxidoreductase Bile | | |
| acid biosynthesis EC: 1.1.1.50 | | |
| RE2625 | CYP27A1 | −0.09 |
| peroxisomal thiolase 2 | SCP2 | −0.09 |
| lipid, flip-flop intracellular transport | | −0.09 |
| Very-long-chain-fatty-acid-CoA ligase | SLC27A2 | −0.09 |
| lipid, flip-flop intracellular transport | | −0.09 |
| lipid, flip-flop intracellular transport | | −0.09 |
| exchange reaction for tetradecanoate (n- | | −0.09 |
| C14: 0) | | |
| methylmalonyl-CoA mutase | MUT | −0.09 |
| urate export from peroxisome | | −0.09 |
| xanthine diffusion in peroxisome | | −0.09 |
| xanthine oxidase, peroxisomal | XDH | −0.09 |
| CO2 transporter via diffusion | | −0.09 |
| carboxylic acid dissociation | CAR1; CAR12; | −0.09 |
| | CAR13; CAR14; | |
| | CAR2; CAR3; | |
| | CAR4; CAR6; | |
| | CAR7; CAR9 | |
| Carbonic acid hydro-lyase Nitrogen | CAR1; CAR12; | −0.09 |
| metabolism EC: 4.2.1.1 | CAR13; CAR14; | |
| | CAR2; CAR4; | |
| | CAR6; CAR7; | |
| | CAR8; CAR9 | |
| NADPH transporter, peroxisome | | −0.09 |
| NADP transporter, peroxisome | | −0.09 |
| o2 transport (diffusion) | | −0.09 |
| RE2658 | | −0.09 |
| Beta oxidation of long chain fatty acid | ACADM; ACADS | −0.09 |
| phosphatidylinositol 3-phosphate 4-kinase | PI4K2A; PI4KA; | −0.09 |
| | PI4KB; PIP5K1A; | |
| | PIP5K1B; | |
| | PIP5K1C | |
| D-Lactaldehyde: NAD+ 1-oxidoreductase | AKR7A5 | −0.09 |
| Propane-1,2-diol: NAD+ 1-oxidoreductase | AKR7A5 | −0.09 |
| chondroitin sulfate B proteoglycan protease, | | −0.09 |
| lysosome (endosome) | | |
| chondroitin sulfate B transport, extracellular | | −0.09 |
| to lysosome | | |
| chondroitin sulfate B/dermatan sulfate | | −0.09 |
| (IdoA2S-GalNAc4S) proteoglycan exchange | | |
| alpha-L-iduronidase, lysosomal | IDUA | −0.09 |
| beta-N-acetylhexosaminidase, lysosomal | HEXA; HEXB | −0.09 |
| beta-N-acetylhexosaminidase A, lysosomal | HEXA; HEXB | −0.09 |
| iduronate-2-sulfatase, lysosomal | IDS | −0.09 |
| N-acetylgalactosamine-4-sulfatase, | ARSB | −0.09 |
| lysosomal | | |
| chondroitin sulfate A proteoglycan protease, | | −0.09 |
| lysosome (endosome) | | |
| chondroitin sulfate C proteoglycan protease, | | −0.09 |
| lysosome (endosome) | | |
| chondroitin sulfate A transport, extracellular | | −0.09 |
| to lysosome | | |
| chondroitin sulfate C transport, extracellular | | −0.09 |
| to lysosome | | |
| chondroitin sulfate A (GalNAc4S-GlcA) | | −0.09 |
| proteoglycan exchange | | |
| chondroitin sulfate C (GalNAc6S-GlcA) | | −0.09 |
| proteoglycan exchange | | |
| beta-glucuronidase, lysosomal | GUSB | −0.09 |
| beta-glucuronidase, lysosomal | GUSB | −0.09 |
| beta-N-acetylhexosaminidase, lysosomal | HEXA; HEXB | −0.09 |
| beta-N-acetylhexosaminidase, lysosomal | HEXA; HEXB | −0.09 |
| beta-N-acetylhexosaminidase, lysosomal | HEXA; HEXB | −0.09 |
| beta-N-acetylhexosaminidase A, lysosomal | HEXA; HEXB | −0.09 |
| beta-N-acetylhexosaminidase A, lysosomal | HEXA; HEXB | −0.09 |
| beta-N-acetylhexosaminidase A, lysosomal | HEXA; HEXB | −0.09 |
| N-acetylgalactosamine-4-sulfatase, | ARSB | −0.09 |
| lysosomal | | |
| N-acetylgalactosamine-6-sulfatase, | CTSA; GALNS; | −0.09 |
| lysosomal | GLB1; NEU1 | |
| N-acetylgalactosamine-6-sulfatase, | CTSA; GALNS; | −0.09 |
| lysosomal | GLB1; NEU1 | |
| RE2658 | | −0.09 |
| TCDB: 2.A.1.13.5 TCDB: 2.A.1.13.1 | SLC16A1; | −0.09 |
| | SLC16A7 | |
| TCDB: 2.A.1.13.5 TCDB: 2.A.1.13.1 | SLC16A1; | −0.09 |
| | SLC16A7 | |
| RE0453 | NME4 | −0.09 |
| NADH dehydrogenase, mitochondrial | 1700029P11RIK; | −0.09 |
| | MT-ND1; MT-ND2; | |
| | MT-ND3; MT-ND4; | |
| | MT-ND4L; MT- | |
| | ND5; MT-ND6; | |
| | NDUFA1; | |
| | NDUFA10; | |
| | NDUFA11; | |
| | NDUFA12; | |
| | NDUFA13; | |
| | NDUFA2; | |
| | NDUFA3; | |
| | NDUFA4; | |
| | NDUFA5; | |
| | NDUFA6; | |
| | NDUFA7; | |
| | NDUFA8; | |
| | NDUFA9; | |
| | NDUFAB1; | |
| | NDUFB10; | |
| | NDUFB2; | |
| | NDUFB3; | |
| | NDUFB4; | |
| | NDUFB5; | |
| | NDUFB6; | |
| | NDUFB7; | |
| | NDUFB8; | |
| | NDUFB9; | |
| | NDUFC1; | |
| | NDUFC2; NDUFS1; | |
| | NDUFS2; NDUFS3; | |
| | NDUFS4; NDUFS5; | |
| | NDUFS6; NDUFS7; | |
| | NDUFS8; NDUFV1; | |
| | NDUFV2; | |
| | NDUFV3; TUSC3 | |
| Ceramide kinase | CERK | −0.09 |
| crmp hs transport | | −0.09 |
| Ceramide 1-phosphate exchange | | −0.09 |
| exchange reaction for Pyridoxal | | −0.09 |
| Pyridoxal transport | | −0.09 |
| guanine phosphoribosyltransferase | HPRT | −0.09 |
| hypoxanthine phosphoribosyltransferase | HPRT | −0.09 |
| (Hypoxanthine) | | |
| N-acylsphingosine amidohydrolase | ASAH1 | −0.09 |
| Galactocerebrosidase | GALC | −0.09 |
| galactocerebroside intracellular transport | | −0.09 |
| RE2677 | | −0.09 |
| fatty acid intracellular transport | | −0.09 |
| sphingosine intracellular transport | | −0.09 |
| N-Acetylneuraminate 9-phosphate pyruvate- | NANS | −0.09 |
| lyase (pyruvate-phosphorylating) | | |
| N-Acetylneuraminate 9-phosphate | ACP5; ACP6; | −0.09 |
| phosphohydrolase | NANP | |
| N-Acetylneuraminate lyase (reversible) | | −0.09 |
| N-acetyl-D-mannosamine kinase | GNE; NAGK | −0.09 |
| H2O transport, nuclear | | −0.09 |
| Propanoyl-CoA: (acceptor) 2,3- | ACADM | −0.09 |
| oxidoreductase beta-Alanine metabolism | | |
| EC: 1.3.3.6 EC: 1.3.99.3 | | |
| formate dehydrogenase | ALDH1L1 | −0.09 |
| RE2655 | | −0.09 |
| elaidic acid exchange | | −0.09 |
| fatty-acid--CoA ligase | ACSL1 | −0.09 |
| Maltose exchange | | −0.09 |
| dUTP diphosphatase | | −0.09 |
| 10-Formyltetrahydrofolate mitochondrial | | −0.09 |
| transport via diffusion | | |
| 2-Oxoadipate: lipoamde 2- | OGDH | −0.09 |
| oxidoreductase(decarboxylating and | | |
| acceptor-succinylating) Lysine degradation | | |
| EC: 1.2.4.2 | | |
| Glutaryl-CoA: dihydrolipoamide S- | DLST | −0.09 |
| succinyltransferase Lysine degradation | | |
| EC: 2.3.1.61 | | |
| phosphogluconate dehydrogenase | PGD | −0.09 |
| RE1796 | HSD3B2 | −0.09 |
| thiamin phosphatase | | −0.09 |
| Thiamin pyrophosphokinase (EC 2.7.6.2) | TPK1 | −0.09 |
| Tyrosine: dopa oxidase | TYR | −0.09 |
| cytochrome P450, family 7, subfamily A, | CYP7A1 | −0.09 |
| polypeptide 1 | | |
| RE2814 | | −0.09 |
| 27 hydroxy cholesterol transport | | −0.09 |
| UTP exchange | | −0.09 |
| UDP-GlcNAc: betaGal beta-1,3-N- | B3GNT3 | −0.09 |
| acetylglucosaminyltransferase 3, Golgi | | |
| apparatus | | |
| ceramide transport protein | COL4A3BP | −0.09 |
| galgluside hs intracellular transport | | −0.09 |
| galgluside hs intracellular transport | | −0.09 |
| Glucosylceramidase | GBA | −0.09 |
| Beta-galactosidase | CTSA; GALNS; | −0.09 |
| | GLB1; NEU1 | |
| glucose efflux from lysosome | | −0.09 |
| RE1521 | ECHS1; EHHADH; | −0.09 |
| | HADHA; HADHB | |
| RE1526 | EHHADH; HADH; | −0.09 |
| | HSD17B10; | |
| | HSD17B4 | |
| RE1533 | ACAA2; HADHA; | −0.09 |
| | HADHB | |
| RE1573 | ECI1; ECI3; | −0.09 |
| | EHHADH | |
| RE2999 | ECI1; ECI3; | −0.09 |
| | EHHADH | |
| RE3000 | ECH1 | −0.09 |
| RE3001 | DECR1 | −0.09 |
| RE3003 | ECI1; ECI3; | −0.09 |
| | EHHADH | |
| RE3004 | ECHS1; EHHADH; | −0.09 |
| | HADHA; HADHB | |
| RE3005 | EHHADH; HADH; | −0.09 |
| | HSD17B10; | |
| | HSD17B4 | |
| RE3006 | ACAA2; HADHA; | −0.09 |
| | HADHB | |
| RE3624 | ACOX1 | −0.09 |
| ALAALACNc | CNDP1; CNDP2 | −0.09 |
| transport of Alanylalanine by the apical | SLC15A1 | −0.09 |
| PEPT1 amino acid transporters across the | | |
| brush border cells of the enterocytes of the | | |
| intestine and renal cells | | |
| exchange reaction for alanylalanine | | −0.09 |
| transport of 2-methylcrotonoyl-CoA into | DBI | −0.09 |
| cytosol | | |
| production of tiglylcarnitine | CPT1A; CPT1B; | −0.09 |
| | CPT1C | |
| exchange reaction for tiglyl carnitine | | −0.09 |
| transport of tiglyl carnitine into the extra | | −0.09 |
| cellular fluid | | |
| Glycerol: NADP+ oxidoreductase Glycerolipid | AKR1A1; AKR1B3 | −0.09 |
| metabolism EC: 1.1.1.72 EC: 1.1.1.2 | | |
| ATP: (R)-glycerate 3-phosphotransferase | GLYCTK | −0.09 |
| Glycine, serine and threonine metabolism | | |
| EC: 2.7.1.31 | | |
| D-Glyceraldehyde: NAD+ oxidoreductase | ALDH1B1; | −0.09 |
| Glycerolipid metabolism EC: 1.2.1.3 | ALDH2; | |
| | ALDH3A2; | |
| | ALDH7A1; | |
| | ALDH9A1 | |
| Postulated transport reaction | | −0.09 |
| ATP pyrophosphohydrolase Purine | ENTPD1; ENTPD3; | −0.09 |
| metabolism EC: 3.6.1.5 | ENTPD8 | |
| alcohol dehydrogenase (D-lactaldehyde) | ADH1; ADH4; | −0.09 |
| | ADH5; ADH6A; | |
| | ADH7; ADHFE1; | |
| | ZADH2 | |
| D-Lactaldehyde: NAD+ oxidoreductase | ADH5 | −0.09 |
| fglutathione-formylating) | | |
| lactoylglutathione lyase | GLO1 | −0.09 |
| carnitine C22: 6 transferase | CPT1A; CPT1B; | −0.09 |
| | CPT1C | |
| C226 transport into the mitochondria | CPT2 | −0.09 |
| C226 transport into the mitochondria | | −0.09 |
| Beta oxidation of long chain fatty acid | ACADM; ACADS | −0.09 |
| 4-Pyridoxal secretion | | −0.10 |
| 4-Pyridoxate exchange | | −0.10 |
| pyridoxal dehydrogenase | A0X1 | −0.10 |
| hyaluronan exchange | | −0.10 |
| uptake of docosanoic acid by cells by | | −0.10 |
| diffusion | | |
| transport of docosanoic acid by diffusion | | −0.10 |
| excretion of docosanedioic acid | | −0.10 |
| exchange reaction for Behenic acid | | −0.10 |
| exchange reaction for docosanedioic acid | | −0.10 |
| fatty acid omega oxidation(C22-->w- | CYP4F15 | −0.10 |
| OHC22)er | | |
| fatty acid omega oxidation(w-OHC22-->C22DC)c | ADH5; ALDH3A2 | −0.10 |
| transport of w-hydroxydocosanoic acid by | | −0.10 |
| diffusion | | |
| fatty acid activation(C15)x | SLC27A2 | −0.10 |
| fatty acid activation(C16br)x | SLC27A2 | −0.10 |
| Diphosphate transporter, peroxisome | | −0.10 |
| peroxisomal acyl-CoA thioesterase | ACOT2 | −0.10 |
| asparagine synthase (glutamine- | ASNS | −0.10 |
| hydrolysing) | | |
| L-Asparagine amidohydrolase Alanine and | ASPG | −0.10 |
| aspartate metabolism/Cyanoamino acid | | |
| metabolism/Nitrogen metabolism | | |
| EC: 3.5.1.1 EC: 3.5.1.38 | | |
| hydroxysteroid (17-beta) dehydrogenase 4 | HSD17B4 | −0.10 |
| oxaloacetate decarboxylase | | −0.10 |
| Phosphoenolpyruvate carboxykinase (GTP) | PCK1 | −0.10 |
| omega hydroxy tetradecanoate (n-C14: 0) | | −0.10 |
| exchange | | |
| Fatty acid omega-hydroxylase | CYP4A29 | −0.10 |
| xenobiotic transport | | −0.10 |
| alpha-methylacyl-CoA racemase (reductase) | AMACR | −0.10 |
| bile acid Coenzyme A: amino acid N- | BAAT | −0.10 |
| acyltransferase | | |
| CO2 peroxisomal transport | | −0.10 |
| peroxisomal thiolase 2 | SCP2 | −0.10 |
| deoxycytidine deaminase, nuclear | A1CDA | −0.10 |
| deoxyuridine transport in nucleus | | −0.10 |
| dCTP diffusion in nucleus | | −0.10 |
| aspartate-glutamate mitochondrial shuttle | SLC25A12; | −0.10 |
| | SLC25A13 | |
| aspartate transaminase | GOT1 | −0.10 |
| aspartate transaminase | GOT2 | −0.10 |
| malate dehydrogenase | MDH1; MDH1B | −0.10 |
| malate dehydrogenase, mitochondrial | MDH1; MDH2 | −0.10 |
| Hypotaurine oxidase | | −0.10 |
| Transport of 5-S-methyl-5-thioadenosine | | −0.10 |
| 2,3-diketo-5-methylthio-1-phosphopentane | | −0.10 |
| degradation reaction | | |
| Exchange of 5-S-methyl-5-thioadenosine | | −0.10 |
| Formate exchange | | −0.10 |
| 5-Methylthio-5-deoxy-D-ribulose 1- | | −0.10 |
| phosphate dehydratase | | |
| 5′-methylthioadenosine phosphorylase | MTAP | −0.10 |
| 5-methylthioribose-1-phosphate isomerase | | −0.10 |
| dATP transport via dCDP antiport | SLC25A19 | −0.10 |
| dATP transport via dUDP antiport | SLC25A19 | −0.10 |
| dATP transport via dTDP antiport | SLC25A19 | −0.10 |
| dATP transport via dADP antiport | SLC25A19 | −0.10 |
| dTMP kinase in mitochondria | DTYMK | −0.10 |
| dATP transport via dGDP antiport | SLC25A19 | −0.10 |
| ceramide transport protein | COL4A3BP | −0.10 |
| choline phosphate intracellular transport | | −0.10 |
| sphingomyelin phosphodiesterase 3, neutral | ENPP7; SMPD3; | −0.10 |
| membrane (neutral sphingomyelinase II) | SMPD4 | |
| Sphingomyelin synthase (homo sapiens) | SGMS1 | −0.10 |
| sphingomyelin intracellular transport | | −0.10 |
| peroxisomal acyl-CoA thioesterase | ACOT2 | −0.10 |
| Proline transport (sodium symport) (2:1) | SLC6A7 | −0.10 |
| phosphatidylinositol 4-phosphate 3-kinase | HCST; PIK3C2A; | −0.10 |
| | PIK3C2B; | |
| | PIK3C2G; PIK3CA; | |
| | PIK3CB; PIK3CD; | |
| | PIK3CG; PIK3R1; | |
| | PIK3R2; PIK3R3; | |
| | PIK3R5 | |
| Asparagine transport (Na, H coupled) | SLC38A3; | −0.10 |
| | SLC38A5 | |
| acetaldehyde reversible transport | | −0.10 |
| Acetaldehyde exchange | | −0.10 |
| Postulated transport reaction | | −0.10 |
| transport of Farnesyl diphosphate into the | | −0.10 |
| endoplasmic reticulum | | |
| digalside hs intracellular transport | | −0.10 |
| galactocerebroside intracellular transport | | −0.10 |
| galactose efflux from lysosome | | −0.10 |
| galactosidase, alpha | GLA | −0.10 |
| RE2666 | | −0.10 |
| hydroxylated testosterone transport | | −0.10 |
| hydroxylated testosterone transport | | −0.10 |
| acetaldehyde reversible transport (ER) | | −0.10 |
| 6 beta hydroxy testosterone exchange | | −0.10 |
| Testosterone exchange | | −0.10 |
| 17-beta-hydroxysteroid dehydrogenase | HSD17B2 | −0.10 |
| testicular 17-beta-hydroxysteroid | HSD17B3 | −0.10 |
| dehydrogenase | | |
| 3 beta-hydroxysteroid dehydrogenase/delta | HSD3B2 | −0.10 |
| 5-->4-isomerase type I | | |
| 3 beta-hydroxysteroid dehydrogenase/delta | HSD3B2 | −0.10 |
| 5-->4-isomerase type I | | |
| 3 beta-hydroxysteroid dehydrogenase/delta | HSD3B2 | −0.10 |
| 5-->4-isomerase type I | | |
| Cytochrome P450 17A1 | CYP17A1 | −0.10 |
| Cytochrome P450 17A1 | CYP17A1 | −0.10 |
| Cytochrome P450 17A1 | CYP17A1 | −0.10 |
| Cytochrome P450 17A1 | CYP17A1 | −0.10 |
| cytochrome p450 P450 3A7 | CYP3A13 | −0.10 |
| pregnenolone intracellular transport | | −0.10 |
| Testosterone transport | | −0.10 |
| testosterone intracellular transport | | −0.10 |
| 11-deoxycortisol intracellular transport | | −0.10 |
| 11-deoxycortisol intracellular transport | | −0.10 |
| 11-deoxycorticosterone intracellular | | −0.10 |
| transport | | |
| 11-deoxycorticosterone intracellular | | −0.10 |
| transport | | |
| aldosterone transport | | −0.10 |
| aldosterone intracellular transport | | −0.10 |
| cortisol transport | | −0.10 |
| cortisol intracellular transport | | −0.10 |
| corticosterone transport | | −0.10 |
| corticosterone intracellular transport | | −0.10 |
| estradiol transport | | −0.10 |
| estrone intracellular transport | | −0.10 |
| Aldosterone exchange | | −0.10 |
| cortisol Exchange | | −0.10 |
| corticosterone Exchange | | −0.10 |
| estradiol exchange | | −0.10 |
| 4,17 dihydroxy estradiol exchange | | −0.10 |
| FOR transporter, endoplasmic reticulum | | −0.10 |
| hydroxylated estrogen derivative transport | | −0.10 |
| hydroxylated estrogen derivative transport | | −0.10 |
| testicular 17-beta-hydroxysteroid | H2-KE6; HSD17B1 | −0.10 |
| dehydrogenase | | |
| Steroid 11-beta-hydroxylase | CYP11B1 | −0.10 |
| Steroid 11-beta-hydroxylase | CYP11B1 | −0.10 |
| Steroid 11-beta-hydroxylase | CYP11B1 | −0.10 |
| aromatase | CYP19A1 | −0.10 |
| aromatase | CYP19A1 | −0.10 |
| cytochrome P450 1B1 | CYP1B1; CYP3A13 | −0.10 |
| Steroid 21-hydroxylase | CYP21A1 | −0.10 |
| Steroid 21-hydroxylase | CYP21A1 | −0.10 |
| glucose 6-phosphate dehydrogenase | G6PD2 | −0.10 |
| diphopshate transporter, mitochondrial | | −0.10 |
| 24,25-Dihydroxyvitamin D3 transport from | | −0.10 |
| mitochondria | | |
| 24R-Vitamin D-25-hydroxylase (D3) | CYP24A1 | −0.10 |
| tanslocation of 1-alpha,25-Dihydroxyvitamin | | −0.10 |
| D3 to nucleus | | |
| 24,25-Dihydroxyvitamin D3 transport from | | −0.10 |
| cytoplasm | | |
| 1-alpha hydroxylation of 25-hydroxy vitamin | CYP27B1 | −0.10 |
| D | | |
| demand reaction for vitD3 | | −0.10 |
| 24R,25-Dihydroxyvitamin D3 exchange | | −0.10 |
| RE2240 | | −0.10 |
| Core 6 beta-GlcNAc-transferase A, Golgi | GCNT1 | −0.10 |
| apparatus | | |
| F1alpha transport (from golgi to lysosome) | | −0.10 |
| beta-galactosidase, lysosomal | CTSA; GALNS; | −0.10 |
| | GLB1; NEU1 | |
| GalNAc transferase, Golgi apparatus | GALNT1; | −0.10 |
| | GALNT10; | |
| | GALNT11; | |
| | GALNT12; | |
| | GALNT13; | |
| | GALNT14; | |
| | GALNT15; | |
| | GALNT2; GALNT3; | |
| | GALNT4; GALNT5; | |
| | GALNT6; GALNT7; | |
| | GALNT9 | |
| N-acetylgalactosamine 4-beta- | | −0.10 |
| galactosyltransferase, Golgi apparatus | | |
| N-acetylgalactosaminidase, alpha- | NAGA | −0.10 |
| N-acetylglucosaminidase, lysosomal | | −0.10 |
| Ser/Thr transport (from golgi to lysosome) | | −0.10 |
| udpacgal intracellular transport | | −0.10 |
| udp intracellular transport | | −0.10 |
| acyl-Coenzyme A oxidase 2, branched chain | ACOX2 | −0.10 |
| alpha-methylacyl-CoA racemase | AMACR | −0.10 |
| phosphatidylinositol synthase | CD1PT | −0.10 |
| (Homo sapiens) | | |
| phosphatidylglycerol (homo sapiens) | | −0.10 |
| exchange | | |
| phosphatidylglycerol transport | | −0.10 |
| Phosphatidylglycerol phosphate | | −0.10 |
| phosphatase (homo sapiens) | | |
| phosphatidyl-CMP: glycerophosphate | PGS1 | −0.10 |
| phosphatidyltransferase | | |
| Ethanolamine phosphate demand | | −0.10 |
| Sphingosine-l-phosphate lyase | SGPL1 | −0.10 |
| Estrone 3-sulfate exchange | | −0.10 |
| glucuronidated compound transport | | −0.10 |
| exchange reaction for blirubin mono- | | −0.10 |
| glucuronide | | |
| UDP-glucuronosyltransferase 1-10 | UGT1A8 | −0.10 |
| precursor, microsomal | | |
| stearoyl-CoA desaturase (n-C18: 0CoA −> n- | SCD4 | −0.10 |
| C18: 1CoA) | | |
| vaccenic acid exchange | | −0.10 |
| fatty-acid--CoA ligase | ACSL1 | −0.10 |
| fatty acid transport via diffusion | | −0.10 |
| inositol-1,4,5-trisphosphate 5-phosphatase | 1NPP5A; INPP5B; | −0.10 |
| | 1NPP5D; INPP5E; | |
| | 1NPP5J; INPPL1; | |
| | SYNJ1 | |
| phosphatidylinositol 4,5-bisphosphate | PLCB1; PLCB2; | −0.10 |
| phospholipase C | PLCB3; PLCB4; | |
| | PLCD1; PLCD3; | |
| | PLCD4; PLCE1; | |
| | PLCG1; PLCG2; | |
| | PLCHI; PLCH2; | |
| | PLCL1; PLCXD2; | |
| | PLCZ1 | |
| N-acetyllactosaminide beta-1,3-N- | B3GNT2; B3GNT3; | −0.10 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.10 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.10 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.10 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.10 |
| acetylglucosaminyltransferase, Golgi | B3GNT4; B3GNT7; | |
| apparatus | B3GNT8; | |
| | B3GNTL1; | |
| | B4GAT1 | |
| N-acetyllactosaminide beta-1,3-N- | B3GNT2; B3GNT3; | −0.10 |
| acetylglucosaminyltransferase, Golgi | B3GNT4; B3GNT7; | |
| apparatus | B3GNT8; | |
| | B3GNTL1; | |
| | B4GAT1 | |
| Core 2 acetylglucosaminyltransferase, Golgi | GCNT1; GCNT3; | −0.10 |
| apparatus | GCNT4 | |
| Core 3 beta-GlcNAc-transferase, Golgi | B3GNT6 | −0.10 |
| apparatus | | |
| Core 4 beta6-GalNAc-transferase, Golgi | GCNT3 | −0.10 |
| apparatus | | |
| beta-N-acetylglucosaminylglycopeptide beta- | B4GALT1; | −0.10 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.10 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.10 |
| 1.4-ealactosvltransferase. Golgi | R4GAI.T2: | |
| | B4GALT3; | |
| | B4GALT5 | |
| beta-N-acetylglucosaminylglycopeptide beta- | B4GALT1; | −0.10 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.10 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.10 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.10 |
| 1,4-galactosyltransferase, Golgi | B4GALT2; | |
| | B4GALT3; | |
| | B4GALT5 | |
| beta-N-acetylglucosaminylglycopeptide beta- | B4GALT1; | −0.10 |
| 1,4-galactosyltransferase, Golgi | B4GALT2; | |
| | B4GALT3; | |
| | B4GALT5 | |
| beta-galactosidase, lysosomal | CTSA; GALNS; | −0.10 |
| | GLB1; NEU1 | |
| beta-galactosidase, lysosomal | CTSA; GALNS; | −0.10 |
| | GLB1; NEU1 | |
| beta-galactosidase, lysosomal | CTSA; GALNS; | −0.10 |
| | GLB1; NEU1 | |
| beta-galactosidase, lysosomal | CTSA; GALNS; | −0.10 |
| | GLB1; NEU1 | |
| keratan sulfate II (core2) transport, golgi to | | −0.10 |
| extracellular | | |
| keratan sulfate II (core 2) transport, | | −0.10 |
| extracellular to lysosome | | |
| keratan sulfate II (core4) transport, golgi to | | −0.10 |
| extracellular | | |
| keratan sulfate II (core 4) transport, | | −0.10 |
| extracellular to lysosome | | |
| N-acetylgalactosamine 3-beta- | C1GALT1 | −0.10 |
| galactosyltransferase, Golgi apparatus | | |
| beta-N-acetylhexosaminidase, lysosomal | HEXA; HEXB | −0.10 |
| beta-N-acetylhexosaminidase, lysosomal | HEXA; HEXB | −0.10 |
| beta-N-acetylhexosaminidase, lysosomal | HEXA; HEXB | −0.10 |
| beta-N-acetylhexosaminidase, lysosomal | HEXA; HEXB | −0.10 |
| beta-N-acetylhexosaminidase A, lysosomal | HEXA; HEXB | −0.10 |
| beta-N-acetylhexosaminidase A, lysosomal | HEXA; HEXB | −0.10 |
| beta-N-acetylhexosaminidase A, lysosomal | HEXA; HEXB | −0.10 |
| beta-galactoside alpha-2,3-sialyltransferase | ST3GAL1; | −0.10 |
| (core 2) | ST3GAL2; | |
| | ST3GAL4 | |
| beta-galactoside alpha-2,3-sialyltransferase | ST3GAL1; | −0.10 |
| | ST3GAL2; | |
| | ST3GAL4 | |
| galactose/N-acetylglucosamine 6-O- | CHST2; CHST4; | −0.10 |
| sulfotransferase, Golgi apparatus | CHST5 | |
| galactose/N-acetylglucosamine 6-O- | CHST2; CHST4; | −0.10 |
| sulfotransferase, Golgi apparatus | CHST5 | |
| galactose/N-acetylglucosamine 6-O- | CHST1; CHST3 | −0.10 |
| sulfotransferase, Golgi apparatus | | |
| galactose/N-acetylglucosamine 6-O- | CHST2; CHST4; | −0.10 |
| sulfotransferase, Golgi apparatus | CHST5 | |
| galactose/N-acetylglucosamine 6-O- | CHST2; CHST4; | −0.10 |
| sulfotransferase, Golgi apparatus | CHST5 | |
| galactose/N-acetylglucosamine 6-O- | CHST1; CHST3 | −0.10 |
| sulfotransferase, Golgi apparatus | | |
| galactose-6-sulfate sulfatase, lysosomal | CTSA; GALNS; | −0.10 |
| | GLB1; NEU1 | |
| N-acetylglucosamine-6-sulfatase, lysosomal | GNS | −0.10 |
| N-acetylglucosamine-6-sulfatase, lysosomal | GNS | −0.10 |
| galactose-6-sulfate sulfatase, lysosomal | CTSA; GALNS; | −0.10 |
| | GLB1; NEU1 | |
| N-acetylglucosamine-6-sulfatase, lysosomal | GNS | −0.10 |
| sialidase, lysosomal | CTSA; GALNS; | −0.10 |
| | GLB1; NEU1 | |
| sialidase, lysosomal | CTSA; GALNS; | −0.10 |
| | GLB1; NEU1 | |
| fatty acid beta oxidation(C18: 5-->C16: 4)m | ACADVL; HADHA; | −0.10 |
| | HADHB | |
| isomerization(C18: 5)m | ECU | −0.10 |
| fatty acid beta oxidation(C20: 5-->C18: 5)m | ACADVL; HADHA; | −0.10 |
| | HADHB | |
| fatty acid beta oxidation(C22: 6-->C20: 5)m | ACADVL; HADHA; | −0.10 |
| | HADHB | |
| fatty acid beta oxidation(C22: 6-->C22: 7)m | ACADVL | −0.10 |
| isomerization(C22: 6)m | ECU | −0.10 |
| fatty acid beta oxidation(C22: 7-->C22: 6)m | DECR1 | −0.10 |
| phosphoribosylpyrophosphate synthetase | PRPS1; PRPS1L1; | −0.10 |
| | PRPS2 | |
| adenine phosphoribosyltransferase | APRT | −0.10 |
| 13-cis-retinoyl glucuronide exchange | | −0.10 |
| RE2147 | UGT1A1 | −0.10 |
| retinoyl glucuronide efflux (13-cis) | | −0.10 |
| Glycylproline exchange | | −0.10 |
| exchange reaction for prolyl-glycine | | −0.10 |
| transport of Glycylproline by the apical | SLC15A1 | −0.10 |
| PEPT1 amino acid transporters across the | | |
| brush border cells of the enterocytes of the | | |
| intestine and renal cells | | |
| hydrolysis of Prolylglycine in the small | PEPD | −0.10 |
| intestine for cellular uptake | | |
| transport of Prolylglycine by the apical | SLC15A1 | −0.10 |
| PEPT1 amino acid transporters across the | | |
| brush border cells of the enterocytes of the | | |
| intestine and renal cells | | |
| hydrolysis of glycylproline in the small | PEPD | −0.10 |
| intestine for cellular uptake | | |
| exchange reaction for NO | | −0.10 |
| GMP exchange | | −0.10 |
| lipid, flip-flop intracellular transport | | −0.10 |
| acetaldehyde peroxisomal diffusion | | −0.10 |
| catalase A, peroxisomal (ethanol) | CAT | −0.10 |
| ethanol reversible peroxisomal transport | | −0.10 |
| glycine reversible transport via sodium and | SLC6A9 | −0.10 |
| chloride symport (2:1:1) | | |
| 5,6,7,8-Tetrahydrofolate transport, diffusion, | | −0.10 |
| mitochondrial | | |
| dUDP nuclear transport | | −0.10 |
| dUMP nuclear transport | | −0.10 |
| dUTP diphosphatase, nuclear | DUT | −0.10 |
| nucleoside-diphosphate kinase (ATP: dUDP), | GM20390; NME2 | −0.10 |
| nuclear | | |
| aspartate 1-decarboxylase | GAD1 | −0.10 |
| L-arabinoase extracellular transport | | −0.10 |
| arabinose reductase | AKR1A1; AKR1B3; | −0.10 |
| | AKR7A5 | |
| L-Arabinose exchange | | −0.10 |
| GTP: pyruvate O2-phosphotransferase Purine | PKLR | −0.10 |
| metabolism EC: 2.7.1.40 | | |
| CTP: pyruvate O2-phosphotransferase | PKLR | −0.10 |
| EC: 2.7.1.40 | | |
| UTP: pyruvate O2-phosphotransferase | PKLR | −0.10 |
| EC: 2.7.1.40 | | |
| dATP: pyruvate O2-phosphotransferase | PKLR | −0.10 |
| Purine metabolism EC: 2.7.1.40 | | |
| dATP: pyruvate O2-phosphotransferase | PKLR | −0.10 |
| Purine metabolism EC: 2.7.1.40 | | |
| L-alanine reversible transport via proton | SLC36A1; | −0.10 |
| symport | SLC36A2 | |
| deoxycytidine transport via diffusion | SLC29A2 | −0.10 |
| exchange reaction for Deoxycytidine | | −0.10 |
| 2-keto-4-methylthiobutyrate transamination | | −0.10 |
| Nad(p)h biliverdin reductase | BLVRA; BLVRB | −0.10 |
| CO transporter via diffusion | | −0.10 |
| Bilirubin exchange | | −0.10 |
| Carbon monoxide exchange | | −0.10 |
| EX_fe2(u) | | −0.10 |
| Fe3+ exchange | | −0.10 |
| exchange reaction for heme | | −0.10 |
| Heme oxygenase 1 | HMOX1; HMOX2 | −0.10 |
| dehydroascorbate reductase | GLRX; GLRX2 | −0.10 |
| dehydroascorbate transport (uniport) | SLC2A1; SLC2A3; | −0.10 |
| | SLC2A4 | |
| Urate exchange | | −0.10 |
| Xanthine: oxygen oxidoreductase Purine | XDH | −0.10 |
| metabolism EC: 1.17.3.2 | | |
| urate export from cytosol | | −0.10 |
| Prostaglandin E1 exchange | | −0.10 |
| RE3568 | | −0.10 |
| agmatinase (m) | AGMAT | −0.10 |
| arginine decarboxylase (m) | AZIN2 | −0.10 |
| Active transport | | −0.10 |
| FAD diphosphatase | ENPP1; ENPP3 | −0.11 |
| FMN adenylyltransferase | FLAD1 | −0.11 |
| ethanol monooxygenase | CYP2E1 | −0.11 |
| deoxyadenosine transport via diffusion | SLC29A2 | −0.11 |
| deoxycytidine transport via diffusion | SLC29A2 | −0.11 |
| deoxyuridine transport via diffusion | SLC29A2 | −0.11 |
| Concentrative Nucleoside Transporter (CNT) | SLC28A1; | −0.11 |
| TCDB: 2.A.41.2.3 | SLC28A3 | |
| Concentrative Nucleoside Transporter (CNT) | SLC28A1; | −0.11 |
| TCDB: 2.A.41.2.3 | SLC28A3 | |
| Concentrative Nucleoside Transporter (CNT) | SLC28A1; | −0.11 |
| TCDB: 2.A.41.2.3 | SLC28A3 | |
| pyridoxine 5′-phosphate oxidase | PNPO | −0.11 |
| pyridoxamine 5′-phosphate oxidase | PNPO | −0.11 |
| Pyridoxamine: oxygen oxidoreductase | PNPO | −0.11 |
| (deaminating) Vitamin B6 metabolism | | |
| EC: 1.4.3.5 | | |
| Pyridoxine: oxygen oxidoreductase | PNPO | −0.11 |
| (deaminating) Vitamin B6 metabolism | | |
| EC: 1.1.3.12 | | |
| adenine reversible transport, cytosol | SLC29A2 | −0.11 |
| Vitamin D-25-hydroxylase (D3) | CYP27A1 | −0.11 |
| Retinol transport via facilitated diffusion | RBP1; RBP2; | −0.11 |
| | RBP4; STRA6 | |
| L-asparagine transport in via sodium | ACE2; SLC38A1; | −0.11 |
| symport | SLC38A2; | |
| | SLC38A4; | |
| | SLC6A14; | |
| | SLC6A19; | |
| | TMEM27 | |
| purine-nucleoside phosphorylase (Inosine) | PNP2 | −0.11 |
| RE2675 | | −0.11 |
| Sarcosine transport (mitochondrial) | | −0.11 |
| Sarcosine dehydrogenase (ni) | SARDH | −0.11 |
| RE2814 | | −0.11 |
| 02 transport (diffusion) | | −0.11 |
| lipid, flip-flop intracellular transport | | −0.11 |
| arginase | ARG1; ARG2 | −0.11 |
| transport of GABA | | −0.11 |
| Demand for 4-Aminobutanoate(n) | | −0.11 |
| hypochlorous acid exchange | | −0.11 |
| exchange reaction for Chloride | | −0.11 |
| RE2513 | LPO | −0.11 |
| exchange reaction for sulfocysteine | | −0.11 |
| Formation of sulfocysteine | | −0.11 |
| Exit of sulfocysteine into extra-cellular space | | −0.11 |
| carbamoyl-phosphate synthase (ammonia) | CPS1 | −0.11 |
| (mitochondria | | |
| 5′-nucleotidase (IMP) | NT5C1A; NT5C1B; | −0.11 |
| | NT5C2; NT5C3; | |
| | NT5E | |
| exchange reaction for dpcoa | | −0.11 |
| acetol monooxygenase | CYP2E1 | −0.11 |
| RE3012 | ALDH3A1; | −0.11 |
| | ALDH3A2 | |
| RE3015 | CYP4F15; | −0.11 |
| | CYP4F39 | |
| RE3016 | | −0.11 |
| RE3017 | | −0.11 |
| Sulfate derivative of norepinephrine | | −0.11 |
| exchange | | |
| norepinephrine sulfate transport (diffusion) | | −0.11 |
| Norepinephrine Sulfotransferase | SULT1A1 | −0.11 |
| 6-glutamyl-10FTHF transport, lysosomal | | −0.11 |
| 6-glutamyl-DHF transport, lysosomal | | −0.11 |
| 6-glutamyl-THF transport, lysosomal | | −0.11 |
| folylpolyglutamate synthetase | FPGS | −0.11 |
| folylpolyglutamate synthetase (DHF) | FPGS | −0.11 |
| folylpolyglutamate synthetase (10fthf) | FPGS | −0.11 |
| Gamma-glutamyl hydrolase (10FTHF6GLU), | GGH | −0.11 |
| lysosomal | | |
| Gamma-glutamyl hydrolase (6DHF), | GGH | −0.11 |
| lysosomal | | |
| Gamma-glutamyl hydrolase (6THF), | GGH | −0.11 |
| lysosomal | | |
| 5-glutamyl-10FTHF transport, lysosomal | | −0.11 |
| 10-Formyltetrahydrofolate lysosomal | | −0.11 |
| transport via diffusion | | |
| 5-glutamyl-DHF transport, lysosomal | | −0.11 |
| 5-glutamyl-THF transport, lysosomal | | −0.11 |
| dihydrofolate reversible lysosomal transport | | −0.11 |
| folylpolyglutamate synthetase | FPGS | −0.11 |
| folylpolyglutamate synthetase | FPGS | −0.11 |
| folylpolyglutamate synthetase (DHF) | FPGS | −0.11 |
| folylpolyglutamate synthetase (DHF) | FPGS | −0.11 |
| folylpolyglutamate synthetase (lOfthf) | FPGS | −0.11 |
| folylpolyglutamate synthetase (lOfthf) | FPGS | −0.11 |
| Gamma-glutamyl hydrolase (10FTHF5GLU), | GGH | −0.11 |
| lysosomal | | |
| Gamma-glutamyl hydrolase (5DHF), | GGH | −0.11 |
| lysosomal | | |
| Gamma-glutamyl hydrolase (5THF), | GGH | −0.11 |
| lysosomal | | |
| Glutamate transport, lysosomal | | −0.11 |
| H2O transport, lysosomal | | −0.11 |
| 5,6,7,8-Tetrahydrofolate transport, diffusion, | | −0.11 |
| lysosomal | | |
| Glutathione dehydrogenase | | −0.11 |
| (dehydroascorbate reductase) | | |
| Mitochondrial Carrier (MC) TCDB: 2.A.29.21.1 | | −0.11 |
| Mitochondrial Carrier (MC) TCDB: 2.A.29.19.1 | SLC25A2 | −0.11 |
| Mitochondrial Carrier (MC) TCDB: 2.A.29.19.1 | SLC25A2 | −0.11 |
| Mitochondrial Carrier (MC) TCDB: 2.A.29.19.1 | SLC25A2 | −0.11 |
| Mitochondrial Carrier (MC) TCDB: 2.A.29.19.1 | SLC25A2 | −0.11 |
| Mitochondrial Carrier (MC) TCDB: 2.A.29.19.1 | SLC25A2 | −0.11 |
| Mitochondrial Carrier (MC) TCDB: 2.A.29.19.1 | SLC25A2 | −0.11 |
| Facilitated diffusion | | −0.11 |
| Oxidized glutathione exchange | | −0.11 |
| glutathione peroxidase (e) | GPX1; GPX2; | −0.11 |
| | GPX3; GPX6; GPX7 | |
| Nicotinamide-D-ribonucleotide | | −0.11 |
| amidohydrolase Nicotinate and nicotinamide | | |
| metabolism EC: 3.5.1.42 | | |
| D-Alanine Oxidase (x) | DAO | −0.11 |
| D-Alanine transport to perixosome | | −0.11 |
| IMP dehydrogenase | IMPDH1; IMPDH2 | −0.11 |
| NADH: guanosine-5-phosphate | GMPR; GMPR2 | −0.11 |
| oxidoreductase(deaminating) Purine | | |
| metabolism EC: 1.7.1.7 | | |
| Xanthosine-5-phosphate: ammonia ligase | GMPS | −0.11 |
| (AMP-forming) Purine metabolism EC: 6.3.4.1 | | |
| N-acetylglucosamine 2-epimerase | RENBP | −0.11 |
| RE3524 | | −0.11 |
| lysophosphatidylcholine transport | | −0.11 |
| diacylglycerol transport | | −0.11 |
| fatty acid retinol efflux | | −0.11 |
| retinyl ester hydrolase, extracellular | | −0.11 |
| RTOTAL2 transport | | −0.11 |
| dihydrofolate reversible mitochondrial | | −0.11 |
| transport | | |
| 5,6,7,8-Tetrahydrofolate: NADP+ | DHFR | −0.11 |
| oxidoreductase One carbon pool by folate/ | | |
| Folate biosynthesis EC: 1.5.1.3 | | |
| 5,6,7,8-Tetrahydrofolate transport, diffusion, | | −0.11 |
| mitochondrial | | |
| L-Proline exchange | | −0.11 |
| Exchange of formaldehyde | | −0.11 |
| Free diffusion | | −0.11 |
| 5′-nucleotidase (GMP) | NT5C1A; NT5C1B; | −0.11 |
| | NT5C2; NT5C3; | |
| | NT5E | |
| fatty acyl-CoA desaturase (n-C18: 2CoA −> n- | FADS1; FADS2 | −0.11 |
| C18: 3CoA) | | |
| linoleic acid (all cis C18: 2) exchange | | −0.11 |
| xanthine dehydrogenase, peroxisomal | XDH | −0.11 |
| Facilitated diffusion | | −0.11 |
| L-dopachrome isomerase 1 | TYR | −0.11 |
| deoxyguanylate kinase (dGMP: ATP) | GUK1 | −0.12 |
| dihomo-gamma-linolenic acid (n-6) | | −0.12 |
| exchange | | |
| fatty-acid--CoA ligase | ACSL1 | −0.12 |
| Cholate: CoA ligase (AMP-forming) Bile acid | SLC27A5 | −0.12 |
| biosynthesis EC: 6.2.1.7 | | |
| Active transport | | −0.12 |
| Postulated transport reaction | | −0.12 |
| RE1834 | ACOT2; ACOT6; | −0.12 |
| | ACOT8; BAAT | |
| Amino Acid-Polyamine-Organocation (APC) | SLC7A5 | −0.12 |
| TCDB: 2.A.3.8.1 | | |
| Amino Acid-Polyamine-Organocation (APC) | SLC7A5 | −0.12 |
| TCDB: 2.A.3.8.1 | | |
| Amino Acid-Polyamine-Organocation (APC) | SLC7A5 | −0.12 |
| TCDB: 2.A.3.8.1 | | |
| Amino Acid-Polyamine-Organocation (APC) | SLC7A5 | −0.12 |
| TCDB: 2.A.3.8.1 | | |
| Amino Acid-Polyamine-Organocation (APC) | SLC7A5 | −0.12 |
| TCDB: 2.A.3.8.1 | | |
| Amino Acid-Polyamine-Organocation (APC) | SLC7A5 | −0.12 |
| TCDB: 2.A.3.8.1 | | |
| Amino Acid-Polyamine-Organocation (APC) | SLC7A5 | −0.12 |
| TCDB: 2.A.3.8.1 | | |
| Amino Acid-Polyamine-Organocation (APC) | SLC7A5 | −0.12 |
| TCDB: 2.A.3.8.1 | | |
| Amino Acid-Polyamine-Organocation (APC) | SLC7A5 | −0.12 |
| TCDB: 2.A.3.8.1 | | |
| Amino Acid-Polyamine-Organocation (APC) | SLC7A5 | −0.12 |
| TCDB: 2.A.3.8.1 | | |
| xenobiotic transport | | −0.12 |
| xenobiotic transport | | −0.12 |
| xenobiotic transport | | −0.12 |
| xenobiotic transport | | −0.12 |
| xenobiotic transport | | −0.12 |
| xenobiotic transport | | −0.12 |
| xenobiotic transport | | −0.12 |
| xenobiotic transport | | −0.12 |
| debrisoquine uniport | SLC22A2 | −0.12 |
| xenobiotic transport | | −0.12 |
| 25-Hydroxyvitamin D3 exchange | | −0.12 |
| 4 hydroxy debrisoquine exchange | | −0.12 |
| 4 hydroxy tolbutamide exchange | | −0.12 |
| 5 hydroxy omeprazole exchange | | −0.12 |
| aflatoxin B1 exchange | | −0.12 |
| alpha-Pinene-oxide exchange | | −0.12 |
| (+)-alpha-Pinene exchange | | −0.12 |
| (E)-carveol exchange | | −0.12 |
| coumarin exchange | | −0.12 |
| debrisoquine exchange | | −0.12 |
| 8,9 epoxy aflatoxin B1 exchange | | −0.12 |
| laurate exchange | | −0.12 |
| hydroxy coumarin exchange | | −0.12 |
| hydroxylated taxol exchange | | −0.12 |
| Limonene exchange | | −0.12 |
| Naphthalene exchange | | −0.12 |
| omeprazole exchange | | −0.12 |
| naphthalene epoxide exchange | | −0.12 |
| Perillyl alcohol exchange | | −0.12 |
| paclitaxel exchange | | −0.12 |
| tolbutamide exchange | | −0.12 |
| omega hydroxy dodecanoate (n-C12: 0) | | −0.12 |
| exchange | | |
| Fatty acid omega-hydroxylase | CYP4A29 | −0.12 |
| xenobiotic transport | | −0.12 |
| xenobiotic transport | | −0.12 |
| xenobiotic transport | | −0.12 |
| xenobiotic transport | | −0.12 |
| xenobiotic transport | | −0.12 |
| xenobiotic transport | | −0.12 |
| cytochrome P450 2A6 | CYP2A4 | −0.12 |
| cytochrome P450 2C19 | | −0.12 |
| cytochrome P450 2C8 | | −0.12 |
| cytochrome P450 2C9 | | −0.12 |
| cytochrome P450 2C9 | | −0.12 |
| cytochrome P450 2C9 | | −0.12 |
| cytochrome P450 2C9 | | −0.12 |
| cytochrome P450 2D6 | CYP2D26 | −0.12 |
| cytochrome P450 2F1 | CYP2F2 | −0.12 |
| cytochrome P450 3A5 | CYP3A13 | −0.12 |
| xenobiotic transport | | −0.12 |
| xenobiotic transport | | −0.12 |
| xenobiotic transport | | −0.12 |
| xenobiotic transport | | −0.12 |
| Hexadecanoate (n-C16: 0) exchange | | −0.12 |
| dATP transport via ADP antiport | SLC25A19 | −0.12 |
| dATP transport via ATP antiport | SLC25A19 | −0.12 |
| N-acetyl-galactosamine intracellular | | −0.12 |
| transport | | |
| acgbgbside hs intracellular transport | | −0.12 |
| acgbgbside hs intracellular transport | | −0.12 |
| UDP-GlcNAc: betaGal beta-1,3-N- | B3GNT3 | −0.12 |
| acetylglucosaminyltransferase 3, Golgi | | |
| apparatus | | |
| N-acetylgalactosaminidase, beta- | | −0.12 |
| inositol-1,3,4,5-trisphosphate 5-phosphatase | INPP5A; INPP5B; | −0.12 |
| | INPP5D; INPP5E; | |
| | INPP5J; INPPL1; | |
| | SYNJ1 | |
| inositol-1,3,4-trisphosphate 1-phosphatase | INPP1 | −0.12 |
| inositol-1,4,5-trisphosphate 3-kinase | ITPKA; ITPKB; | −0.12 |
| | ITPKC | |
| inositol-3,4-bisphosphate 4-phosphatase | INPP4A; INPP4B | −0.12 |
| myo-inositol 3-phosphatase | IMPA1; IMPA2 | −0.12 |
| sphingosylphosphorylcholine | | −0.12 |
| (homo sapiens)exchange | | |
| sphingosylphosphorylcholine transport | | −0.12 |
| (diffusion) | | |
| sphingomyelin deacylase | | −0.12 |
| Propanoyl-CoA: acetyl-CoA C-acyltransferase | ACAA1B; ACAA2; | −0.12 |
| Bile acid biosynthesis EC: 2.3.1.16 | HADHB | |
| RE1836 | SCP2 | −0.12 |
| glucuronidated compound transport | | −0.12 |
| Bilirubin beta-diglucuronide exchange | | −0.12 |
| UDP-glucuronosyltransferase 1-10 | UGT1A1; UGT1A2; | −0.12 |
| precursor, microsomal | UGT1A7C; | |
| | UGT1A8 | |
| HYPTROXe | | −0.12 |
| RE1530 | TK2 | −0.12 |
| thiamin pyrophosphatase, mitochondrial | | −0.12 |
| Thiamine monophosphate transport, | | −0.12 |
| mitochondrial | | |
| Thiamine diphosphate transport in via anion | | −0.12 |
| antiport, mitochondria | | |
| diacylglycerol acyltransferase | DGAT1; DGAT2 | −0.12 |
| lipase | CEL; LIPC; LPL; | −0.12 |
| | PNPLA3 | |
| stearidonic acid exchange | | −0.12 |
| exchange reaction for Pyridoxamine | | −0.12 |
| pyridoxamine transport via diffusion | | −0.12 |
| Pyridoxamine: oxygen oxidoreductase | PNPO | −0.12 |
| (deaminating) Vitamin B6 metabolism | | |
| EC: 1.4.3.5 | | |
| Mitochondrial Carrier (MC) TCDB: 2.A.29.7.2 | SLC25A1 | −0.12 |
| Mitochondrial Carrier (MC) TCDB: 2.A.29.7.2 | SLC25A1 | −0.12 |
| Mitochondrial Carrier (MC) TCDB: 2.A.29.7.2 | SLC25A1 | −0.12 |
| Mitochondrial Carrier (MC) TCDB: 2.A.29.7.2 | SLC25A1 | −0.12 |
| Mitochondrial Carrier (MC) TCDB: 2.A.29.7.2 | SLC25A1 | −0.12 |
| citrate transport, mitochondrial | SLC25A1 | −0.12 |
| citrate transport, mitochondrial | SLC25A1 | −0.12 |
| Mitochondrial Carrier (MC) TCDB: 2.A.29.7.2 | SLC25A1 | −0.12 |
| Mitochondrial Carrier (MC) TCDB: 2.A.29.7.2 | SLC25A1 | −0.12 |
| Mitochondrial Carrier (MC) TCDB: 2.A.29.7.2 | SLC25A1 | −0.12 |
| Mitochondrial Carrier (MC) TCDB: 2.A.29.7.2 | SLC25A1 | −0.12 |
| Mitochondrial Carrier (MC) TCDB: 2.A.29.7.2 | SLC25A1 | −0.12 |
| N-Formyl-L-kynurenine amidohydrolase | AFMID | −0.12 |
| L-Tryptophan: oxygen 2,3-oxidoreductase | IDO1; IDO2; TDO2 | −0.12 |
| (decyclizing) | | |
| glutathione peroxidase, mitochondria | GPX1; GPX4; | −0.12 |
| | PRDX3 | |
| glutathione: NAD+ oxidoreductase Glutamate | GSR | −0.12 |
| metabolism EC: 1.8.1.7 | | |
| 4-Aminobutanoate exchange | | −0.12 |
| exchange reaction for D-Fructose | | −0.12 |
| glycine passive transport to peroxisome | | −0.12 |
| xylulokinase | XYLB | −0.12 |
| xylitol dehydrogenase (D-xyulose-forming) | | −0.12 |
| nucleoside-diphosphate kinase (ATP: dADP), | NME4; NME6 | −0.12 |
| mitochondrial | | |
| (R)-3-Hydroxybutanoate: NAD+ | BDH1 | −0.12 |
| oxidoreductase | | |
| (R)-3-Hydroxybutanoate mitochondrial | | −0.12 |
| transport via H+ symport | | |
| (R)-3-Hydroxybutanoate transport via H+ | | −0.12 |
| symport | | |
| L-histidine transport via diffusion | SLC38A4 | −0.12 |
| (extracellular to cytosol) | | |
| trans-Hexadec-2-enoyl-CoA reductase Fatty | MECR; PECR | −0.12 |
| acid elongation in mitochondria EC: 1.3.1.38 | | |
| Palmitoyl-CoA: oxygen 2-oxidoreductase | ACADL; ACADM; | −0.12 |
| Fatty acid metabolism EC: 1.3.3.6 EC: 1.3.99.3 | ACADVL; ACOX1; | |
| | ACOX3 | |
| gamma-glutamylcyclotransferase | GGCT | −0.12 |
| g-glutamyltransferase (e) | GGT1 | −0.12 |
| Mitochondrial Carrier (MC) TCDB: 2.A.29.19.1 | SLC25A2 | −0.12 |
| mannose-6-phosphate isomerase | MP1 | −0.12 |
| methylmalonyl-CoA epimerase/racemase | MCEE | −0.12 |
| methylmalonyl-CoA mutase | MUT | −0.12 |
| Demand for L-cystine | | −0.12 |
| cystathionine g-lyase | CTH | −0.12 |
| cystathionine beta-synthase | CBS | −0.12 |
| transport of palmitoyl coA into the | FABP1 | −0.12 |
| enterocytes | | |
| acetone transport via proton symport | SLC16A1; | −0.12 |
| | SLC16A3; | |
| | SLC16A7 | |
| Acetone exchange | | −0.12 |
| Glycylleucine exchange | | −0.12 |
| exchange reaction for leucylglycine | | −0.12 |
| hydrolysis of Glycylleucine in the small | CNDP2; DPEP1 | −0.12 |
| intestine for cellular uptake | | |
| transport of Glycylleucine by the apical | SLC15A1 | −0.12 |
| PEPT1 amino acid transporters across the | | |
| brush border cells of the enterocytes of the | | |
| intestine and renal cells | | |
| hydrolysis of leucylglycine | | −0.12 |
| transport of Leucylglycine by the apical | SLC15A1 | −0.12 |
| PEPT1 amino acid transporters across the | | |
| brush border cells of the enterocytes of the | | |
| intestine and renal cells | | |
| kinetensin 1-7 exchange | | −0.12 |
| RE2269 | CMA1 | −0.12 |
| Transport of L-Histidine into the cell coupled | SLC38A5 | −0.12 |
| with co-transport with Sodium and counter | | |
| transport with proton by SNAT5 transporter. | | |
| superoxide anion transport via diffusion | | −0.12 |
| (extracellular) | | |
| Sphingosine 1-phosphate exchange | | −0.12 |
| sphingosine-1-phosphate transport | | −0.12 |
| sphingomyelinase | SMPD2 | −0.12 |
| Vesicular transport | | −0.12 |
| 4-Hydroxyphenylpyruvate: oxygen | HPD | −0.12 |
| oxidoreductase | | |
| fumarylacetoacetase | FAH | −0.12 |
| Homogentisate: oxygen 1,2-oxidoreductase | HGD | −0.12 |
| (decyclizing) | | |
| maleylacetoacetate isomerase | GSTZ1 | −0.12 |
| dihydropyrimidinase (5,6-dihydrouracil) | DPYS; DPYSL2; | −0.12 |
| | DPYSL3 | |
| b-ureidopropionase | UPB1 | −0.12 |
| phosphopentomutase | PGM1; PGM2 | −0.12 |
| purine-nucleoside phosphorylase | PNP2 | −0.12 |
| (Adenosine) | | |
| nucleoside-diphosphate kinase (ATP: dUDP), | NME4; NME6 | −0.12 |
| mitochondrial | | |
| GMP synthase | GMPS | −0.12 |
| hydroxypyruvate decarboxylase | | −0.12 |
| hydrogen peroxide peroxisomal transport | | −0.12 |
| via diffusion | | |
| O2 transport, peroxisomal | | −0.12 |
| hydrogen-peroxide: hydrogen-peroxide | CAT | −0.12 |
| oxidoreductase EC: 1.11.1.6 | | |
| alanyl aminopeptidase (cys-gly) | ANPEP | −0.12 |
| glutathione synthetase | GSS | −0.12 |
| gamma-glutamyltranspeptidase Glutathione | GGT1; GGT5; | −0.12 |
| metabolism EC: 3.4.11.4 | GGT6; GGT7 | |
| S-Aminomethyldihydrolipoylprotein: (6S)- | AMT | −0.12 |
| tetrahydrofolate aminomethyltransferase | | |
| (ammonia-forming) One carbon pool by | | |
| folate EC: 2.1.2.10 | | |
| 5-Formyltetrahydrofolate cyclo-ligase (ADP- | MTHFSL | −0.12 |
| forming) One carbon pool by folate | | |
| EC: 6.3.3.2 | | |
| Transport of L-Glutamine into the intestinal | SLC6A14 | −0.12 |
| cells by ATBO transporter | | |
| glycine N-methyltransferase | GNMT | −0.12 |
| Bicarbonate exchange | | −0.12 |
| fatty-acid--CoA ligase | SLC27A2 | −0.12 |
| pristcoa peroxisomal transport | | −0.12 |
| prist peroxisomal transport | | −0.12 |
| diphosphomevalonate decarboxylase, cytosol | MVD | −0.12 |
| mevalonate kinase (atp) cytosol | MVK | −0.12 |
| phosphomevalonate kinase, cytosol | PMVK | −0.12 |
| (R)-Mevalonate: NADP+ oxidoreductase (CoA | HMGCR | −0.12 |
| acylating) Biosynthesis of steroids | | |
| EC: 1.1.1.34 | | |
| Transport of S-adenosyl-L-homocysteine | | −0.12 |
| Exchange of S-adenosyl-L-homocysteine | | −0.12 |
| Arylsulfatase A | ARSA | −0.12 |
| Galactosylceramide sulfotransferase | GAL3ST1 | −0.12 |
| galactocerebroside intracellular transport | | −0.12 |
| 3′-Phosphoadenylyl sufate Golgi transport | SLC35B2 | −0.12 |
| adenosine 3′,5′-bisphosphate Golgi transport | | −0.12 |
| sgalside hs intracellular transport | | −0.12 |
| sgalside hs intracellular transport | | −0.12 |
| Sulfate transport (lysosome) | | −0.12 |
| transport of dATP into mitochondria | | −0.12 |
| xanthine: NAD+ oxidoreductase Purine | XDH | −0.13 |
| metabolism EC: 1.17.1.4 | | |
| Propionate transport, diffusion | | −0.13 |
| dADP transport via ADP antiport | SLC25A19 | −0.13 |
| 2-Deoxyuridine 5-diphosphate: oxidized- | RRM1; RRM2; | −0.13 |
| thioredoxin 2-oxidoreductase Pyrimidine | RRM2B | |
| metabolism EC: 1.17.4.1 | | |
| thioredoxin reductase (NADPH) | TXNRD1 | −0.13 |
| dUDP transport via ADP antiport | SLC25A19 | −0.13 |
| dTDP transport via ADP antiport | SLC25A19 | −0.13 |
| dCDP transport via ADP antiport | SLC25A19 | −0.13 |
| Mitochondrial Carrier (MC) TCDB: 2.A.29.16.1 | SLC25A19 | −0.13 |
| purine-nucleoside phosphorylase | PNP2 | −0.13 |
| (Guanosine) | | |
| RE2655 | | −0.13 |
| Guanine exchange | | −0.13 |
| Guanine transport | SLC29A2 | −0.13 |
| fatty acid beta oxidation(C10-->C8)m | ACAA2; ACADM; | −0.13 |
| | ECHS1; HADH | |
| fatty acid beta oxidation(C12-->C10)m | ACAA2; ACADM; | −0.13 |
| | ECHS1; HADH | |
| (S)-3-Hydroxydodecanoyl-CoA hydro-lyase | ECHS1; EHHADH; | −0.13 |
| Fatty acid elongation in mitochondria/Fatty | HADHA | |
| acid metabolism EC: 4.2.1.17 | | |
| (S)-3-Hydroxydodecanoyl-CoA: NAD+ | EHHADH; HADH; | −0.13 |
| oxidoreductase Fatty acid elongation in | HADHA | |
| mitochondria/Fatty acid metabolism | | |
| EC: 1.1.1.35 EC: 1.1.1.211 | | |
| Decanoyl-CoA: acetyl-CoA C-acyltransferase | ACAA1B; ACAA2; | −0.13 |
| Fatty acid elongation in mitochondria/Fatty | HADHB | |
| acid metabolism EC: 2.3.1.16 | | |
| Glycolaldehyde dehydrogenase | ALDH1A1; | −0.13 |
| | ALDH1A2; | |
| | ALDH1A3; | |
| | ALDH3A1; | |
| | ALDH3A2; | |
| | ALDH3B1; | |
| | ALDH3B3; | |
| | ALDH7A1; | |
| | ALDH9A1 | |
| Formate-tetrahydrofolate ligase | MTHFD1 | −0.13 |
| glyoxylate transport, peroxisomal | | −0.13 |
| Glycolate dehydrogenase (NADP) | GRHPR | −0.13 |
| glycolate transport into peroxisome | | −0.13 |
| Glycolate oxidase, peroxisome | HAO1; HAO2 | −0.13 |
| hydrogen peroxide peroxisomal transport | | −0.13 |
| via diffusion | | |
| O2 transport, peroxisomal | | −0.13 |
| 3-Aminopropanoate: 2-oxoglutarate | ABAT | −0.13 |
| aminotransferase (m) | | |
| Beta-alanine reversible mitochondrial | | −0.13 |
| transport (diffusion) | | |
| methylmalonate-semialdehyde | ALDH6A1 | −0.13 |
| dehydrogenase (malonic semialdehyde), | | |
| mitochondrial | | |
| Succinyl-CoA: glycine C-succinyl- | ALAS1; ALAS2 | −0.13 |
| transferase(decarboxylating) EC: 2.3.1.37 | | |
| Succinyl-CoA: glycine C-succinyl- | ALAS1; ALAS2 | −0.13 |
| transferase(decarboxylating) EC: 2.3.1.37 | | |
| 2-methylcitrate transport via diffusion | | −0.13 |
| 2-Methylcitrate exchange | | −0.13 |
| 2-methylcitrate synthase | | −0.13 |
| nucleoside-diphosphate kinase (ATP: GDP), | NME4; NME6 | −0.13 |
| mitochondrial | | |
| nucleoside-diphosphate kinase (ATP: GDP), | NME4; NME6 | −0.13 |
| mitochondrial | | |
| Itaconate--CoA ligase (GDP-forming), | SUCLG1; SUCLG2 | −0.13 |
| mitochondrial | | |
| Itaconate--CoA ligase (GDP-forming), | SUCLG1; SUCLG2 | −0.13 |
| mitochondrial | | |
| Itaconate--CoA ligase (ADP-forming), | SUCLA2; SUCLG1 | −0.13 |
| mitochondrial | | |
| Itaconate--CoA ligase (ADP-forming), | SUCLA2; SUCLG1 | −0.13 |
| mitochondrial | | |
| mesaconate--CoA ligase (ADP-forming), | SUCLA2; SUCLG1 | −0.13 |
| mitochondrial | | |
| mesaconate--CoA ligase (ADP-forming), | SUCLA2; SUCLG1 | −0.13 |
| mitochondrial | | |
| mesaconate--CoA ligase (GDP-forming) | SUCLG1; SUCLG2 | −0.13 |
| mesaconate--CoA ligase (GDP-forming) | SUCLG1; SUCLG2 | −0.13 |
| Succinate--CoA ligase (GDP-forming) | SUCLG1; SUCLG2 | −0.13 |
| Succinate--CoA ligase (GDP-forming) | SUCLG1; SUCLG2 | −0.13 |
| Succinate--CoA ligase (ADP-forming) | SUCLA2; SUCLG1 | −0.13 |
| Succinate--CoA ligase (ADP-forming) | SUCLA2; SUCLG1 | −0.13 |
| pyrimidine-nucleoside phosphorylase | UPP1; UPP2 | −0.13 |
| (uracil) | | |
| dUDP transport via ATP antiport | SLC25A19 | −0.13 |
| dTDP transport via ATP antiport | SLC25A19 | −0.13 |
| dCDP transport via ATP antiport | SLC25A19 | −0.13 |
| dGDP transport via ATP antiport | SLC25A19 | −0.13 |
| dUTP transport via ADP antiport | SLC25A19 | −0.13 |
| dUTP transport via ATP antiport | SLC25A19 | −0.13 |
| dADP transport via ATP antiport | SLC25A19 | −0.13 |
| beta-Alanine exchange | | −0.13 |
| Acetyl-CoA hydrolase Pyruvate metabolism | ACOT12 | −0.13 |
| EC: 3.1.2.1 | | |
| ceramide transport protein | COL4A3BP | −0.13 |
| galactocerebroside intracellular transport | | −0.13 |
| RE2677 | | −0.13 |
| thymidine transport in via sodium symport | SLC28A1; | −0.13 |
| | SLC28A3 | |
| acetyl-CoA synthetase | ACSS1 | −0.13 |
| 3-oxoacid CoA-transferase | OXCT1; OXCT2B | −0.13 |
| formaldehyde transport via diffusion | | −0.13 |
| (lysosomall) | | |
| hydrogen peroxide lysosomal transport via | | −0.13 |
| diffusion | | |
| Methanol transporter, lysosome | | −0.13 |
| Peroxidase (multiple substrates) | MPO | −0.13 |
| purine-nucleoside phosphorylase | PNP2 | −0.13 |
| (Deoxyadenosine) | | |
| C181 fatty acid activation | ACSBG2; ACSL1; | −0.13 |
| | ACSL3; ACSL4 | |
| RE3245 | | −0.13 |
| Uptake of arachidate by the enterocytes | | −0.13 |
| R group coenzyme a ligase | | −0.13 |
| R group coenzyme a ligase | | −0.13 |
| R group coenzyme a ligase | | −0.13 |
| lipase | CEL; LPL | −0.13 |
| monoacylglycerol acyltransferase | MOGAT1; | −0.13 |
| | MOGAT2 | |
| Nalpha-(beta-alanyl)-L-histidine hydrolase | CNDP1; CNDP2 | −0.13 |
| IR | | |
| R group phosphotase 1 | | −0.13 |
| R group phosphotase 2 | | −0.13 |
| R group phosphotase 3 | | −0.13 |
| L-Histidine: beta-alanine ligase (AMP- | CARNS1 | −0.13 |
| forming) Alanine and aspartate metabolism/ | | |
| Histidine metabolism/beta-Alanine | | |
| metabolism EC: 6.3.2.11 | | |
| RE1835 | ACOT2; ACOT6; | −0.13 |
| | BAAT | |
| retinol acyltransferase | | −0.13 |
| retinyl ester hydrolase | | −0.13 |
| uptake of Rtotal2 by enterocytes | SLC27A4 | −0.13 |
| uptake of Rtotal3 by enterocytes | SLC27A4 | −0.13 |
| RTOTAL3 transport | | −0.13 |
| uptake of Rtotal by enterocytes | SLC27A4 | −0.13 |
| RTOTAL transport | | −0.13 |
| uptake of octadecenoate by the enterocytes | SLC27A4 | −0.13 |
| Chenodeoxycholate: CoA ligase (AMP- | SLC27A5 | −0.13 |
| forming) Bile acid biosynthesis EC: 6.2.1.7 | | |
| C180 fatty acid activation | ACSL1; ACSL3; | −0.13 |
| | ACSL5 | |
| RE0344 | ACOT2; ACOT6; | −0.13 |
| | BAAT | |
| Maltohexaose exchange | | −0.13 |
| sulfite oxidase | SUOX | −0.13 |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| Amino Acid-Polyamine-Organocation (APC) | SLC7A5 | −0.13 |
| TCDB: 2.A.3.8.1 | | |
| cholate transport via bicarbonate | SLCO1A1; | −0.13 |
| countertransport | SLCO1B2 | |
| dCMP deaminase | DCTD | −0.13 |
| deoxycytidine deaminase | AICDA; CDA | −0.13 |
| Nicotinate exchange | | −0.13 |
| phospholipase A2 | PLA2G10; | −0.13 |
| | PLA2G12A; | |
| | PLA2G1B; | |
| | PLA2G2A; | |
| | PLA2G2D; | |
| | PLA2G2E; | |
| | PLA2G2F; PLA2G5 | |
| L-alanine/glutamine reversible exchange | SLC3A2; SLC7A10 | −0.13 |
| L-alanine/glutamine reversible exchange | SLC3A2; SLC7A10 | −0.13 |
| D-alanine/glycine reversible exchange | SLC3A2; SLC7A10 | −0.13 |
| D-alanine/glycine reversible exchange | SLC3A2; SLC7A10 | −0.13 |
| D-Serine/Glutamine reversible exchange | SLC3A2; SLC7A10 | −0.13 |
| D-Serine/Glutamine reversible exchange | SLC3A2; SLC7A10 | −0.13 |
| D-Serine/Glycine reversible exchange | SLC3A2; SLC7A10 | −0.13 |
| D-Serine/Glycine reversible exchange | SLC3A2; SLC7A10 | −0.13 |
| inositol-1,4-bisphosphate 4-phosphatase | | −0.13 |
| 5 alpha dihydrotesterone transport | | −0.13 |
| 5alpha-Dihydrotestosterone exchange | | −0.13 |
| adenylate cyclase | ADCY1; ADCY10; | −0.13 |
| | ADCY2; ADCY3; | |
| | ADCY4; ADCY5; | |
| | ADCY6; ADCY7; | |
| | ADCY8; ADCY9 | |
| 3′,5′-cyclic-nucleotide phosphodiesterase | PDE10A; PDE11A; | −0.13 |
| | PDE1A; PDE1B; | |
| | PDE1C; PDE2A; | |
| | PDE3A; PDE3B; | |
| | PDE4A; PDE4B; | |
| | PDE4C; PDE4D; | |
| | PDE7A; PDE7B; | |
| | PDE8A; PDE8B | |
| exchange reaction for L-phenylalanine | | −0.13 |
| L-Phenylalanine, tetrahydrobiopterin: oxygen | PAH | −0.13 |
| oxidoreductase (4-hydroxylating) | | |
| Phenylalanine, tyrosine and tryptophan | | |
| biosynthesis EC: 1.14.16.1 | | |
| guanosine facilated transport in cytosol | SLC29A1; | −0.13 |
| | SLC29A2 | |
| exchange reaction for Guanosine | | −0.13 |
| Inosine exchange | | −0.13 |
| esterification of cholesterol to cholesterol | SOAT1; SOAT2 | −0.13 |
| ester within enterocytes | | |
| hydrolysis of cholesterol ester by cholesterol | LIPA | −0.13 |
| esterase | | |
| RTOTAL transport | | −0.13 |
| cholesterol ester transporter | | −0.13 |
| fatty-acid--CoA ligase | ACSBG2; ACSL1; | −0.13 |
| | ACSL3; ACSL4 | |
| Transport reaction | | −0.13 |
| C160 fatty acid activation | ACSBG2; ACSL1; | −0.13 |
| | ACSL3; ACSL4; | |
| | ACSL5 | |
| Palmitate: CoA ligase (AMP-forming) Fatty | ACSBG2; ACSL1; | −0.13 |
| acid metabolism EC: 6.2.1.3 | ACSL3; ACSL4; | |
| | ACSL5; ACSL6; | |
| | SLC27A2 | |
| EC: 2.3.1.26 | SOAT1; SOAT2 | −0.13 |
| EC: 2.3.1.26 | SOAT1; SOAT2 | −0.13 |
| EC: 2.3.1.26 | SOAT1; SOAT2 | −0.13 |
| EC: 3.1.1.13 | CEL; LIPA | −0.13 |
| EC: 3.1.1.13 | CEL; LIPA | −0.13 |
| EC: 3.1.1.13 | CEL; LIPA | −0.13 |
| EC: 6.2.1.3 | ACSBG2; ACSL1; | −0.13 |
| | ACSL3; ACSL4; | |
| | ACSL5; ACSL6; | |
| | SLC27A2 | |
| uptake of Hexadecanoate by the enterocytes | SLC27A4 | −0.13 |
| adenine reversible transport, cytosol | SLC29A2 | −0.13 |
| Adenine exchange | | −0.13 |
| Guanine exchange | | −0.13 |
| Hypoxanthine exchange | | −0.13 |
| Guanine transport | SLC29A2 | −0.13 |
| Hypoxanthine transport | SLC29A2 | −0.13 |
| L-glutamate 5-semialdehyde dehydratase | | −0.13 |
| (spontaneous) | | |
| glutamate semi-aldehyde transport, | | −0.13 |
| mitochondrial | | |
| transketolase | TKT; TKTL1; | −0.13 |
| | TKTL2 | |
| transketolase | TKT; TKTL1; | −0.13 |
| | TKTL2 | |
| GABA secretion via secretory vesicle (ATP | SLC32A1 | −0.13 |
| driven) | | |
| methylglyoxal synthase | | −0.14 |
| methyglyoxylate synthase 2 (from g3p) | | −0.14 |
| 2-Deoxyadenosine 5-diphosphate: oxidized- | RRM1; RRM2; | −0.14 |
| thioredoxin 2-oxidoreductase Purine | RRM2B | |
| metabolism EC: 1.17.4.1 | | |
| L-glutamate reversible transport via proton | SLC25A12; | −0.14 |
| symport, mitochondrial | SLC25A13; | |
| | SLC25A18; | |
| | SLC25A22 | |
| L-arabinitol 4-dehydrogenase | | −0.14 |
| fatty acid transport via diffusion | | −0.14 |
| eicosatetranoic acid exchange | | −0.14 |
| fatty-acid--CoA ligase | ACSL1 | −0.14 |
| choline phosphate intracellular transport | | −0.14 |
| sphingomyelin phosphodiesterase 3, neutral | SMPD1; SMPD3 | −0.14 |
| membrane (neutral sphingomyelinase II) | | |
| sphingomyelin intracellular transport | | −0.14 |
| diphosphomevalonate decarboxylase | MVD | −0.14 |
| Isopentenyl diphosphate transport | | −0.14 |
| (peroxisome) | | |
| mevalonate kinase (atp) | MVK | −0.14 |
| phosphomevalonate kinase | PMVK | −0.14 |
| MCT 1 Transport reaction | SLC16A1 | −0.14 |
| argininosuccinate lyase | | −0.14 |
| argininosuccinate synthase | ASS1 | −0.14 |
| L-Methionine exchange | | −0.14 |
| PTPATe | | −0.14 |
| ATP Exporter (ATP-E) TCDB: 9.A.6.1.1 | | −0.14 |
| Methionine/Leucine exchange (Met in) | SLC3A2; SLC7A5 | −0.14 |
| CO2 exchange | | −0.14 |
| Bicarbonate exchange | | −0.14 |
| EC: 1.2.7.2 | BCKDHA; | −0.14 |
| | BCKDHB; DBT; | |
| | DLD | |
| glycerol transport via channel | | −0.14 |
| alpha-glucosidase, extracellular | MGAM | −0.14 |
| Leukotriene C4 carboxypeptidase | | −0.14 |
| guanylate kinase (GMP: ATP) | GUK1 | −0.14 |
| Guanosine 5-triphosphate | ITPA | −0.14 |
| pyrophosphohydrolase Purine metabolism | | |
| EC: 3.6.1.19 | | |
| Amino Acid-Polyamine-Organocation (APC) | SLC7A5 | −0.14 |
| TCDB: 2.A.3.8.1 | | |
| Amino Acid-Polyamine-Organocation (APC) | SLC7A5 | −0.14 |
| TCDB: 2.A.3.8.1 | | |
| Amino Acid-Polyamine-Organocation (APC) | SLC7A5 | −0.14 |
| TCDB: 2.A.3.8.1 | | |
| Amino Acid-Polyamine-Organocation (APC) | SLC7A5 | −0.14 |
| TCDB: 2.A.3.8.1 | | |
| ADPribose exchange | | −0.14 |
| Glycerophosphodiester phosphodiesterase | | −0.14 |
| (Glycerophosphocholine) | | |
| lysophospholipase | PLA2G4A | −0.14 |
| phospholipase A2 | PLA2G12A; | −0.14 |
| | PLA2G2A; | |
| | PLA2G2F; | |
| | PLA2G3; | |
| | PLA2G4A; | |
| | PLA2G4B; | |
| | PLA2G4E; | |
| | PLA2G5; PLA2G6 | |
| dUTP diphosphatase | DUT; ITPA | −0.14 |
| uridylate kinase (dUMP), mitochondrial | DTYMK | −0.14 |
| phosphogluconate dehydrogenase | PGD | −0.14 |
| 6-phosphogluconolactonase | PGLS | −0.14 |
| Thymidine exchange | | −0.14 |
| thymd transport | SLC29A1; | −0.14 |
| | SLC29A2 | |
| beta-Alanine exchange | | −0.14 |
| Exchange reaction for glycylglycine | | −0.14 |
| hydrolysis of glycylycine for uptake | CNDP1; CNDP2 | −0.14 |
| transport of Glycyl-glycine by the apical | SLC15A1 | −0.14 |
| PEPT1 amino acid transporters across the | | |
| brush border cells of the enterocytes of the | | |
| intestine and renal cells | | |
| ribulose 5-phosphate 3-epimerase | RPE | −0.14 |
| RE1804 | CYP27A1 | −0.14 |
| RE1807 | CYP27A1 | −0.14 |
| xenobiotic transport | | −0.14 |
| xenobiotic transport | | −0.14 |
| 2,4 dihydroxy nitrophenol exchange | | −0.14 |
| cytochrome P450 2E1 | CYP2E1 | −0.14 |
| L-alanine/glutamine reversible exchange | SLC3A2; SLC7A10 | −0.14 |
| L-alanine/glutamine reversible exchange | SLC3A2; SLC7A10 | −0.14 |
| L-Serine/Glutamine reversible exchange | SLC3A2; SLC7A10 | −0.14 |
| L-Serine/Glutamine reversible exchange | SLC3A2; SLC7A10 | −0.14 |
| L-threonine/glycine reversible exchange | SLC3A2; SLC7A10 | −0.14 |
| L-threonine/glycine reversible exchange | SLC3A2; SLC7A10 | −0.14 |
| Cyanide transport via diffusion | | −0.14 |
| (mitochondrial) | | |
| Thiosulfate exchange | | −0.14 |
| RE1691 | TST | −0.14 |
| Thiocyanate transport via diffusion | | −0.14 |
| (mitochondrial) | | |
| thiosulfate transport via sodium symport | SLC13A1 | −0.14 |
| L-proline: (acceptor) oxidoreductase | PRODH | −0.14 |
| Arginine and proline metabolism EC: 1.5.99.8 | | |
| ADPribose transport | CD38 | −0.14 |
| arginase (m) | ARG2 | −0.14 |
| Urea transport via diffusion | AQP9 | −0.14 |
| L-cysteine/L-glutamine reversible exchanger | SLC3A2; SLC7A10; | −0.14 |
| | SLC7A5 | |
| L-cysteine/L-glutamine reversible exchanger | SLC3A2; SLC7A10; | −0.14 |
| | SLC7A5 | |
| exchange reaction for Adenosine | | −0.14 |
| Adenosine monophosphate deaminase | AMPD1; AMPD2; | −0.14 |
| | AMPD3 | |
| 5-beta-cytochrome P450, family 27, | CYP27A1 | −0.15 |
| subfamily A, polypeptide 1 | | |
| RE2626 | CYP27A1 | −0.15 |
| Cytochrome P450 27 | CYP27A1 | −0.15 |
| deoxyuridine phosphorylase | PNP2; UPP2 | −0.15 |
| acetone monooxygenase | CYP2E1 | −0.15 |
| Utilized transport | | −0.15 |
| 3-Methylbutanoyl-CoA: (acceptor) 2,3- | ACADM; IVD | −0.15 |
| oxidoreductase Valine, leucine and isoleucine | | |
| degradation EC: 1.3.99.10 | | |
| Nicotinate D-ribonucleotide: pyrophosphate | NAPRT; QPRT | −0.15 |
| phosphoribosyltransferase Nicotinate and | | |
| nicotinamide metabolism EC: 2.4.2.11 | | |
| purine-nucleoside phosphorylase | PNP2 | −0.15 |
| (Adenosine) | | |
| formimidoyltransferase cyclodeaminase | FTCD | −0.15 |
| Glutamate formimidoyltransferase | FTCD | −0.15 |
| histidase | HAL | −0.15 |
| Imidazolonepropionase | AMDHD1 | −0.15 |
| URCN | UROCI | −0.15 |
| fatty-acid--CoA ligase | ACSL1 | −0.15 |
| RE0577 | ACOT2; ACOT6; | −0.15 |
| | BAAT | |
| UMP exchange | | −0.15 |
| dihydroceramide desaturase | DEGS1; DEGS2 | −0.15 |
| dihydroceramide desaturase | DEGS1; DEGS2 | −0.15 |
| dihydrosphingosine N-acyltransferase | | −0.15 |
| cGMP transport (ATP-dependent) | ABCC4; ABCC5 | −0.15 |
| 3′,5′-Cyclic GMP exchange | | −0.15 |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Amino Acid-Polyamine-Organocation (APC) | SLC7A5 | −0.15 |
| TCDB: 2.A.3.8.1 | | |
| Exchange of L-malate | | −0.15 |
| Transport of L-malate | | −0.15 |
| exchange reaction for Deoxyuridine | | −0.15 |
| dCMP deaminase | DCTD | −0.15 |
| 2-Deoxyadenosine 5-diphosphate: oxidized- | RRM1; RRM2; | −0.15 |
| thioredoxin 2-oxidoreductase Purine | RRM2B | |
| metabolism EC: 1.17.4.1 | | |
| ADPribose diphosphatase | NDDT5 | −0.15 |
| NAD nucleosidase | | −0.15 |
| nicotinamide-nucleotide adenylyltransferase | NMNAT2; | −0.15 |
| | NMNAT3 | |
| purine-nucleoside phosphorylase | PNP2 | −0.15 |
| ribosylnicotinamide kinase | NMRK1 | −0.15 |
| 2-Oxopropanal: NADP+ oxidoreductase | | −0.15 |
| Pyruvate metabolism EC: 1.2.1.49 | | |
| digalside hs intracellular transport | | −0.15 |
| RE2666 | B4GALT6 | −0.15 |
| exchange reaction for diglyceride | | −0.15 |
| L-alanine/glycine reversible exchange | SLC3A2; SLC7A10 | −0.15 |
| L-alanine/glycine reversible exchange | SLC3A2; SLC7A10 | −0.15 |
| L-Serine/Glycine reversible exchange | SLC3A2; SLC7A10 | −0.15 |
| L-Serine/Glycine reversible exchange | SLC3A2; SLC7A10 | −0.15 |
| L-threonine/glycine reversible exchange | SLC3A2; SLC7A10 | −0.15 |
| L-threonine/glycine reversible exchange | SLC3A2; SLC7A10 | −0.15 |
| acetate-CoA ligase (AMP-forming) | ACSS1; ACSS3 | −0.15 |
| RE2649 | ACOT2; ACOT7 | −0.15 |
| L-cysteine/glycine reversible exchanger | SLC3A2; SLC7A10; | −0.15 |
| | SLC7A8 | |
| L-cysteine/glycine reversible exchanger | SLC3A2; SLC7A10; | −0.15 |
| | SLC7A8 | |
| glycine passive transport to mitochondria | | −0.15 |
| deoxyguanylate kinase (dGMP: ATP) | GUK1 | −0.15 |
| guanylate kinase (GMP: ATP) | GUK1 | −0.15 |
| purine-nucleoside phosphorylase | PNP2 | −0.15 |
| (Deoxy guanosine) | | |
| Free diffusion | | −0.15 |
| D-Ribose-5-phosphate ketol-isomerase | RPIA | −0.15 |
| Pentose phosphate pathway EC: 5.3.1.6 | | |
| Free diffusion | | −0.15 |
| Free diffusion | | −0.15 |
| Free diffusion | | −0.15 |
| ribose-5-phosphate isomerase | RPIA | −0.15 |
| ribose-5-phosphate isomerase | RPIA | −0.15 |
| D-Ribose-5-phosphate ketol-isomerase | RPIA | −0.15 |
| Pentose phosphate pathway EC: 5.3.1.6 | | |
| Creatine exchange | | −0.15 |
| guanidinoacetate N-methyltransferase (c) | GAMT | −0.15 |
| glycine amidinotransferase (c) | GATM | −0.15 |
| O2 transport (diffusion) | | −0.15 |
| N-Acetylneuraminate 9-phosphate pyruvate- | NANS | −0.15 |
| lyase (pyruvate-phosphorylating) | | |
| DM kdn(c) | | −0.15 |
| 2-keto-3deoxy-D-glycero-D-galactononic | | −0.15 |
| acid phosphohydrolase | | |
| Mitochondrial Carrier (MC) TCDB: 2.A.29.7.2 | SLC25A1 | −0.15 |
| Mitochondrial Carrier (MC) TCDB: 2.A.29.7.2 | SLC25A1 | −0.15 |
| 4-aminobutanoate mitochondrial transport | | −0.16 |
| via diffusion | | |
| 4-aminobutyrate transaminase, reversible | ABAT | −0.16 |
| (mitochondrial) | | |
| Gamma-glutamylcysteine synthetase | GCLC; GCLM | −0.16 |
| (5-L-Glutamyl)-L-amino-acid 5- | GGCT | −0.16 |
| glutamyltransferase (cyclizing) Glutathione | | |
| metabolism EC: 2.3.2.4 | | |
| Free diffusion | | −0.16 |
| 5′-nucleotidase (XMP) | NT5C; NT5C1A; | −0.16 |
| | NT5C1B; NT5C2; | |
| | NT5C3; NT5E | |
| Amino Acid-Polyamine-Organocation (APC) | SLC7A5 | −0.16 |
| TCDB: 2.A.3.8.1 | | |
| Amino Acid-Polyamine-Organocation (APC) | SLC7A5 | −0.16 |
| TCDB: 2.A.3.8.1 | | |
| Amino Acid-Polyamine-Organocation (APC) | SLC7A5 | −0.16 |
| TCDB: 2.A.3.8.1 | | |
| Amino Acid-Polyamine-Organocation (APC) | SLC7A5 | −0.16 |
| TCDB: 2.A.3.8.1 | | |
| Amino Acid-Polyamine-Organocation (APC) | SLC7A5 | −0.16 |
| TCDB: 2.A.3.8.1 | | |
| Amino Acid-Polyamine-Organocation (APC) | SLC7A5 | −0.16 |
| TCDB: 2.A.3.8.1 | | |
| Amino Acid-Polyamine-Organocation (APC) | SLC7A5 | −0.16 |
| TCDB: 2.A.3.8.1 | | |
| Amino Acid-Polyamine-Organocation (APC) | SLC7A5 | −0.16 |
| TCDB: 2.A.3.8.1 | | |
| Amino Acid-Polyamine-Organocation (APC) | SLC7A5 | −0.16 |
| TCDB: 2.A.3.8.1 | | |
| Amino Acid-Polyamine-Organocation (APC) | SLC7A5 | −0.16 |
| TCDB: 2.A.3.8.1 | | |
| Amino Acid-Polyamine-Organocation (APC) | SLC7A5 | −0.16 |
| TCDB: 2.A.3.8.1 | | |
| Amino Acid-Polyamine-Organocation (APC) | SLC7A5 | −0.16 |
| TCDB: 2.A.3.8.1 | | |
| adenylosuccinate lyase | ADSL | −0.16 |
| phosphoribosylaminoimidazole carboxylase | PAICS | −0.16 |
| Phosphoribosylglycinamide | GART | −0.16 |
| formyltransferase | | |
| glutamine phosphoribosyldiphosphate | PPAT | −0.16 |
| amidotransferase | | |
| Phosphoribosylglycinamide synthase | GART | −0.16 |
| phosphoribosylaminoimidazolesuccinocarbo | PAICS | −0.16 |
| xamide synthase | | |
| phosphoribosylformylglycinamidine | PFAS | −0.16 |
| synthase | | |
| 2-(Formamido)-N1-(5- | GART | −0.16 |
| phosphoribosyl)acetamidine cyclo-ligase | | |
| (ADP-forming) Purine metabolism EC: 6.3.3.1 | | |
| deoxyuridine kinase (ATP: Deoxyuridine) | TK1; TK2 | −0.16 |
| RE1530 | | −0.16 |
| 2-oxoisovalerate dehydrogenase (acylating; | BCKDHA; | −0.16 |
| 4-methyl-2-oxopentaoate), mitochondrial | BCKDHB; DBT; | |
| | DLD | |
| deoxyuridine transport via diffusion | SLC29A2 | −0.16 |
| exchange reaction for Deoxyuridine | | −0.16 |
| transport of L-Cysteine into the intestinal | SLC6A14 | −0.16 |
| cells by ATBO transporter | | |
| dihydrofolate reductase | DHFR | −0.16 |
| exchange reaction for Deoxyinosine | | −0.16 |
| exchange reaction for Deoxyguanosine | | −0.16 |
| purine-nucleoside phosphorylase | PNP2 | −0.16 |
| (Deoxyinosine) | | |
| dehydro-L-gulonate decarboxylase | | −0.16 |
| L-gulonate 3-dehydrogenase | | −0.16 |
| RE0383 | AKR1A1 | −0.16 |
| L-proline: (acceptor) oxidoreductase | PRODH | −0.16 |
| Arginine and proline metabolism EC: 1.5.99.8 | | |
| 7-glutamyl-10FTHF transport, lysosomal | | −0.16 |
| 7-glutamyl-DHF transport, lysosomal | | −0.16 |
| 7-glutamyl-THF transport, lysosomal | | −0.16 |
| Gamma-glutamyl hydrolase (10FTHF7GLU), | GGH | −0.16 |
| lysosomal | | |
| Gamma-glutamyl hydrolase (7DHF), | GGH | −0.16 |
| lysosomal | | |
| Gamma-glutamyl hydrolase (7THF), | GGH | −0.16 |
| lysosomal | | |
| Dihydrofolate: NAD+ oxidoreductase Folate | DHFR | −0.16 |
| biosynthesis EC: 1.5.1.3 | | |
| aldehyde dehydrogenase (acetaldehyde, | ALDH1A2; | −0.16 |
| NADP) | ALDH1B1; | |
| | ALDH3A1; | |
| | ALDH3A2; | |
| | ALDH3B1; | |
| | ALDH3B3; | |
| | ALDH9A1 | |
| aldehyde dehydrogenase (acetaldehyde, | ALDH1A1; | −0.16 |
| NAD) | ALDH1A2; | |
| | ALDH1A3; | |
| | ALDH1B1; | |
| | ALDH3A1; | |
| | ALDH3A2; | |
| | ALDH3B1; | |
| | ALDH3B3; | |
| | ALDH7A1; | |
| | ALDH9A1 | |
| pyrimidine-nucleoside phosphorylase | UPP1; UPP2 | −0.16 |
| (uracil) | | |
| ATP synthase (four protons for one ATP) | ATP5A1; ATP5B; | −0.16 |
| | ATP5C1; ATP5D; | |
| | ATP5E; ATP5F1; | |
| | ATP5G1; ATP5G2; | |
| | ATP5G3; ATP5H; | |
| | ATP5J; ATP5J2; | |
| | ATP5K; ATP5O; | |
| | ATP5S; GM5426; | |
| | NSF | |
| acetaldehyde reversible transport | | −0.16 |
| deoxyribose-phosphate aldolase | DERA | −0.16 |
| Acetaldehyde exchange | | −0.16 |
| 2-Deoxy-D-ribose 1-phosphate 1,5- | PGM1 | −0.16 |
| phosphomutase Pentose phosphate pathway | | |
| EC: 5.4.2.7 | | |
| uptake of octadecanoate (n-C18: 0) by the | | −0.16 |
| enterocytes | | |
| transport of L-Alanine into the intestinal cells | SLC6A14 | −0.16 |
| by ATBO transporter | | |
| transport of L-Serine into the intestinal cells | SLC6A14 | −0.16 |
| by ATBO transporter | | |
| Transport of L-Threonine into the intestinal | SLC6A14 | −0.16 |
| cells by ATBO transporter | | |
| NMN synthetase | NAMPT | −0.16 |
| nucleoside-diphosphate kinase (ATP: dTDP) | GM20390; NME2; | −0.16 |
| | NME3; NME6; | |
| | NME7 | |
| pyruvate kinase | PKLR; PKM | −0.16 |
| RE2954 | PKM | −0.16 |
| purine-nucleoside phosphorylase | PNP2 | −0.16 |
| (Xanthosine) | | |
| deoxyuridine phosphorylase | PNP2; UPP2 | −0.16 |
| alloxan exchange | | −0.16 |
| RE2888 | LPO | −0.16 |
| Mitochondrial Carrier (MC) TCDB: 2.A.29.19.1 | SLC25A2 | −0.16 |
| Mitochondrial Carrier (MC) TCDB: 2.A.29.19.1 | SLC25A2 | −0.16 |
| leucine mitochondrial transport | | −0.16 |
| leucine transaminase, mitochondrial | BCAT2 | −0.16 |
| cAMP transport (ATP-dependent) | ABCC4; ABCC5 | −0.16 |
| cAMP exchange | | −0.16 |
| proteolysis of apoC-Lys-btn | | −0.16 |
| Apocarboxylase formation | | −0.16 |
| biotin-[acetyl-CoA-carboxylase] ligase | HLCS | −0.16 |
| biocytin transport, nuclear | | −0.16 |
| biotinidase (biotin) | BTD | −0.16 |
| biotinidase (biotin), nuclear | BTD | −0.16 |
| holocarboxylase synthestase (biotin protein | HLCS | −0.16 |
| ligase) | | |
| Biotin transport, nuclear | | −0.16 |
| L-lysine transport, nuclear | | −0.16 |
| transport of L-Isoleucine by LAT1 in | SLC3A2; SLC7A5 | −0.16 |
| association with 4F2hc, across the apical | | |
| surface of the memebranes. | | |
| transport of L-Valine by LAT1 in association | SLC3A2; SLC7A5 | −0.16 |
| with 4F2hc, across the apical surface of the | | |
| memebranes. | | |
| transport of L-Asparagine into the cell and | SLC3A2; SLC7A8 | −0.16 |
| efflux of L-Phenylalanine out of the cell by | | |
| LAT2 on the basolateral surfaces of kidney | | |
| and intestine. | | |
| transport of L-Tyrosine into the cell and | SLC3A2; SLC7A8 | −0.16 |
| efflux of L-Phenylalanine out of the cell by | | |
| LAT2 on the basolateral surfaces of kidney | | |
| and intestine. | | |
| transport of L-Threonine into the cell and | SLC3A2; SLC7A8 | −0.16 |
| efflux of L-Phenylalanine out of the cell by | | |
| LAT2 on the basolateral surfaces of kidney | | |
| and intestine. | | |
| Transport of L-Leucine into the cell and | SLC3A2; SLC7A8 | −0.16 |
| efflux of L-Phenylalanine out of the cell by | | |
| LAT2 on the basolateral surfaces of kidney | | |
| and intestine. | | |
| transport of L-Cysteine into the cell and | SLC3A2; SLC7A8 | −0.16 |
| efflux of L-Phenylalanine out of the cell by | | |
| LAT2 on the basolateral surfaces of kidney | | |
| and intestine. | | |
| transport of L-Isoleucine into the cell and | SLC3A2; SLC7A8 | −0.16 |
| efflux of L-Phenylalanine out of the cell by | | |
| LAT2 on the basolateral surfaces of kidney | | |
| and intestine. | | |
| transport of L-Valine into the cell and efflux | SLC3A2; SLC7A8 | −0.16 |
| of L-Phenylalanine out of the cell by LAT2 on | | |
| the basolateral surfaces of kidney and | | |
| intestine. | | |
| Transport of L-Leucine into the cell and | SLC3A2; SLC7A8 | −0.16 |
| efflux of L-Phenylalanine out of the cell by | | |
| LAT2 on the basolateral surfaces of kidney | | |
| and intestine. | | |
| lipase, extracellular | LIPC | −0.17 |
| RE1447 | OXSM | −0.17 |
| RE2972 | PI4KA; PI4KB | −0.17 |
| acetaldehyde mitochondrial diffusion | | −0.17 |
| aldehyde dehydrogenase (acetylaldehyde, | ALDH1B1; ALDH2 | −0.17 |
| NAD), mitochondrial | | |
| Uracil exchange | | −0.17 |
| uracil transport via facilated diffusion | SLC29A2 | −0.17 |
| Thioredoxin (ubiquinone 10) reductase | TXNRD1 | −0.17 |
| (NADH) | | |
| deoxyinosine transport via diffusion | SLC29A2 | −0.17 |
| inositol-1,3,4-trisphosphate 4-phosphatase | | −0.17 |
| inositol-1,3-bisphosphate 3-phosphatase | | −0.17 |
| H2O transport, peroxisomal | | −0.17 |
| thymidine phosphorylase | TYMP | −0.17 |
| Deoxyadenosine deaminase | ADA | −0.17 |
| Tetrahydrobiopterin-4a-carbinolamine | PCBD1 | −0.17 |
| dehydratase | | |
| 5-Aminolevulinate mitochondrial transport | | −0.17 |
| coproporphyrinogen oxidase (O2 required) | CPOX | −0.17 |
| Ferrochelatase, mitochondrial | FECH | −0.17 |
| iron (II) transport | | −0.17 |
| hydroxymethylbilane synthase | HMBS | −0.17 |
| Heme transport to cytosol | | −0.17 |
| porphobilinogen synthase | ALAD | −0.17 |
| protoporphyrinogen IX mitochondrial | | −0.17 |
| transport | | |
| protoporphyrinogen oxidase, mitochondrial | PPOX | −0.17 |
| uroporphyrinogen-III synthase | UROS | −0.17 |
| uroporphyrinogen decarboxylase | UROD | −0.17 |
| (uroporphyrinogen III) | | |
| Transport of N-carbamoyl-L-aspartate | | −0.17 |
| Exchange of N-carbamoyl-L-aspartate | | −0.17 |
| glutathione peroxidase | GPX1; GPX2; | −0.17 |
| | GPX4; PRDX1; | |
| | PRDX2 | |
| Mitochondrial Carrier (MC) TCDB: 2.A.29.7.2 | SLC25A1 | −0.17 |
| Mitochondrial Carrier (MC) TCDB: 2.A.29.7.2 | SLC25A1 | −0.17 |
| Aminoacetone: oxygen | AOC3 | −0.17 |
| oxidoreductase(deaminating)(flavin- | | |
| containing) | | |
| Aminoacetone transport (mitochondrial) | | −0.17 |
| L-2-amino-3-oxobutanoate decarboxylation, | | −0.17 |
| mitochondrial (spontaneous) | | |
| glycine C-acetyltransferase | GCAT | −0.17 |
| inositol oxygenase | M10X | −0.17 |
| deoxyribose transport via diffusion | | −0.17 |
| Deoxyribose exchange | | −0.17 |
| alpha-N-Phenylacetyl-L-glutamine exchange | | −0.17 |
| phenylacetate-CoA ligase | | −0.17 |
| Phenethylamine oxidase | AOC1; AOC2; | −0.17 |
| | MAOA; MAOB | |
| Phenylacetyl-CoA: L-glutamine alpha-N- | | −0.17 |
| phenylacetyltransferase | | |
| PHEACGLN extracellular transport via | | −0.17 |
| diffusion | | |
| L-Phenylalanine carboxy-lyase | DDC | −0.17 |
| Aldehyde: NADP+ oxidoreductase | ALDH1A3; | −0.17 |
| Phenylalanine metabolism EC: 1.2.1.5 | ALDH3A1; | |
| | ALDH3B1; | |
| | ALDH3B3 | |
| purine-nucleoside phosphorylase | PNP2 | −0.17 |
| (Deoxyadenosine) | | |
| L-glutamate 5-semialdehyde dehydratase, | | −0.18 |
| reversible, mitochondrial | | |
| Creatine exchange | | −0.18 |
| guanidinoacetate N-methyltransferase (c) | GAMT | −0.18 |
| glycine amidinotransferase (c) | GATM | −0.18 |
| methylcrotonoyl-CoA carboxylase, | MCCC1; MCCC2 | −0.18 |
| mitochondrial | | |
| methylglutaconyl-CoA hydratase | AUH | −0.18 |
| (reversible), mitochondrial | | |
| orotidine-5′-phosphate decarboxylase | UMPS | −0.18 |
| orotate phosphoribosyltransferase | UMPS | −0.18 |
| Succinate exchange | | −0.18 |
| succinate transport via sodium symport | SLC13A2 | −0.18 |
| transport of DHAP into cytosol | | −0.18 |
| glycerophosphate shuttle for transfer of | | −0.18 |
| reducing equivalents | | |
| glycerol-3-phopshate transport, cytoplasm | | −0.18 |
| glycerol-3-phosphate dehydrogenase (FAD), | GPD2 | −0.18 |
| mitochondrial | | |
| triose-phosphate isomerase | TPI1 | −0.18 |
| Demand for (R)-Pantothenate | | −0.18 |
| exchange reaction for cysam | | −0.18 |
| H2O exchange | | −0.18 |
| Phosphate exchange | | −0.18 |
| (R)-Pantothenate exchange | | −0.18 |
| exchange reaction for ptth | | −0.18 |
| PNTEHe | | −0.18 |
| Exchange of L-malate | | −0.18 |
| Transport of L-malate | | −0.18 |
| aspartate carbamoyltransferase (reversible) | CAD | −0.18 |
| carbamoyl-phosphate synthase (glutamine- | CAD | −0.18 |
| hydrolysing) | | |
| proteolysis of apoC-Lys-btn, mitochondrial | | −0.18 |
| Apocarboxylase formation, mitochondrial | | −0.18 |
| biotin-[acetyl-CoA-carboxylase] ligase, | HLCS | −0.18 |
| mitochondrial | | |
| biotinidase (biotin), mitochondrial | BTD | −0.18 |
| holocarboxylase synthestase (biotin protein | HLCS | −0.18 |
| ligase), mitochondrial | | |
| exchange reaction for Pyridoxine | | −0.18 |
| pyridoxine transport via diffusion | | −0.18 |
| Pyridoxine: oxygen oxidoreductase | PNPO | −0.18 |
| (deaminating) Vitamin B6 metabolism | | |
| EC: 1.1.3.12 | | |
| purine-nucleoside phosphorylase | PNP2 | −0.18 |
| (Deoxy guanosine) | | |
| deoxyadenosine transport via diffusion | SLC29A2 | −0.18 |
| exchange reaction for 2-deoxyadenosine | | −0.18 |
| deoyguanosine transport via diffusion | SLC29A2 | −0.18 |
| L-glutamate reversible transport via proton | SLC25A12; | −0.18 |
| symport, mitochondrial | SLC25A13; | |
| | SLC25A18; | |
| | SLC25A22 | |
| Exchange of orotate | | −0.18 |
| 17-beta-hydroxysteroid dehydrogenase | HSD17B2 | −0.18 |
| formate mitochondrial transport | | −0.18 |
| formate-tetrahydrofolate ligase, | MTHFD1; | −0.18 |
| mitochondrial | MTHFD1L | |
| Dopamine exchange | | −0.18 |
| 3alpha,7alpha-Dihydroxy-5beta- | AKR1C6 | −0.18 |
| cholestane: NADP+ oxidoreductase (B- | | |
| specific); 3alpha,7alpha-Dihydroxy-5beta- | | |
| cholestane: NADP+ oxidoreductase Bile acid | | |
| biosynthesis EC: 1.1.1.50 | | |
| aldo-keto reductase family 1, member D1 | AKR1D1 | −0.18 |
| (delta 4-3-ketosteroid-5-beta-reductase) | | |
| RE1810 | | −0.18 |
| RE2814 | | −0.18 |
| RE2814 | | −0.18 |
| 27 hydroxy cholesterol transport | | −0.18 |
| lipid, flip-flop intracellular transport | | −0.18 |
| lipid, flip-flop intracellular transport | | −0.18 |
| Thymine exchange | | −0.18 |
| thymine reversible transport via facilated | SLC29A2 | −0.18 |
| diffusion | | |
| thymidine phosphorylase | TYMP | −0.18 |
| thymidylate synthase | TYMS | −0.18 |
| Thioredoxin (ubiquinone 10) reductase | TXNRD1 | −0.19 |
| (NADPH) | | |
| methionine adenosyltransferase | MATIA; MAT2B | −0.19 |
| glutathione: NAD+ oxidoreductase Glutamate | GSR | −0.19 |
| metabolism EC: 1.8.1.7 | | |
| Glutathione: cystine oxidoreductase | | −0.19 |
| NADPH: CoA-glutathione oxidoreductase | TXNRD1; TXNRD2 | −0.19 |
| EC: 1.8.1.9 | | |
| Mitochondrial Carrier (MC) TCDB: 2.A.29.7.2 | SLC25A1 | −0.19 |
| exchange reaction for Uridine | | −0.19 |
| Major Facilitator(MFS) TCDB: 2.A.1.4.7 | SLC37A1 | −0.19 |
| exchange reaction for D-Galactose | | −0.19 |
| Glycerol exchange | | −0.19 |
| Exchange of Galactosylglycerol | | −0.19 |
| Monoacylglycerol 2 (homo sapiens) | | −0.19 |
| exchange | | |
| R total 2 position exchange | | −0.19 |
| lipase, extracellular | LIPC | −0.19 |
| Galactosylglycerol galactohydrolase | GLA | −0.19 |
| Galactose metabolism EC: 3.2.1.22 EC: 3.2.1.23 | | |
| exchange reaction for glycylsarcosine | | −0.19 |
| Hydrolysis of glycylsarcosine for uptake | TPP1 | −0.19 |
| Transport of Glycylsarcosine by the apical | SLC15A1 | −0.19 |
| PEPT1 amino acid transporters across the | | |
| brush border cells of the enterocytes of the | | |
| intestine, renal cells and brain. | | |
| Deoxyadenosine deaminase, extracellular | ADA | −0.19 |
| transketolase | TKT; TKTL1; | −0.19 |
| | TKTL2 | |
| transketolase | TKT; TKTL1; | −0.19 |
| | TKTL2 | |
| Glycine betaine exchange | | −0.20 |
| Betaine transport (sodium symport) (2:1) | SLC6A12 | −0.20 |
| NAD(P) transhydrogenase | NNT | −0.20 |
| S-Adenosyl-L-methionine: ethanolamine- | | −0.20 |
| phosphate N-methyltransferase | | |
| Glycerophospholipid metabolism | | |
| EC: 2.1.1.103 | | |
| S-Adenosyl-L- | | −0.20 |
| methionine: methylethanolamine phosphate | | |
| N-methyltransferase Glycerophospholipid | | |
| metabolism EC: 2.1.1.103 | | |
| S-Adenosyl-L- | | −0.20 |
| methionine: phosphodimethylethanolamine | | |
| N-methyltransferase Glycerophospholipid | | |
| metabolism EC: 2.1.1.103 | | |
| RTOTAL2 transport | | −0.20 |
| malic enzyme (NAD), mitochondrial | ME2 | −0.20 |
| malic enzyme (NADP), mitochondrial | ME2; ME3 | −0.20 |
| 4,4-dimethyl-5a-cholesta-8,24-dien-3b- | TM7SF2 | −0.20 |
| ol: NADP+ D14-oxidoreductase Biosynthesis | | |
| of steroids EC: 1.3.1.70 | | |
| C-14 sterol reductase | TM7SF2 | −0.20 |
| hydrogen peroxide mitochondrial transport | | −0.20 |
| dopamine beta-monooxygenase | DBH; MOXD1 | −0.20 |
| Norepinephrine exchange | | −0.20 |
| Melatonin: NADP oxidoreductase | CYP1A1; CYP1A2; | −0.20 |
| | CYP1B1 | |
| RE2426 | | −0.20 |
| Urea exchange | | −0.20 |
| methylmalonate-semialdehyde | ALDH6A1 | −0.20 |
| dehydrogenase | | |
| NADH transporter, endoplasmic reticulum | | −0.20 |
| NAD transporter, endoplasmic reticulum | | −0.20 |
| Adenosine deaminase | ADA | −0.20 |
| Adenosine deaminase, extracellular | ADA | −0.20 |
| Adenosine 5-monophosphate | NT5C; NT5C1A; | −0.20 |
| phosphohydrolase Purine metabolism | NT5C1B; NT5C2; | |
| EC: 3.1.3.5 | NT5C3; NT5E; | |
| | NT5M | |
| malate dehydrogenase, mitochondrial | MDH1; MDH2 | −0.20 |
| pyruvate carboxylase | PCX | −0.20 |
| Hypoxanthine exchange | | −0.20 |
| Hypoxanthine transport | SLC29A2 | −0.20 |
| Uncoupling protein | SLC25A14; | −0.20 |
| | SLC25A27; UCP1; | |
| | UCP2; UCP3 | |
| Methylmalonyl-CoA decarboxylase, | MLYCD | −0.20 |
| mitochondrial | | |
| Propionyl-CoA carboxylase, mitochondrial | PCCA; PCCB | −0.20 |
| S-Formylglutathione hydralase | ESD | −0.20 |
| Sulfite: oxygen oxidoreductase Sulfur | suox | −0.21 |
| metabolism EC: 1.8.3.1 | | |
| Adrenaline secretion via secretory vesicle | SLC18A1; | −0.21 |
| (ATP driven) | SLC18A2 | |
| Adrenaline exchange | | −0.21 |
| noradrenaline N-methyltransferase | PNMT | −0.21 |
| Acetyl-Coa carboxylase, beta isoform | ACACB | −0.21 |
| Malonyl-CoA Decarboxylase, mitochondrial | MLYCD | −0.21 |
| Free diffusion | | −0.21 |
| adenosylhomocysteinase | AHCYL1; GM4737 | −0.21 |
| methionine synthase | MTR | −0.21 |
| adenosine kinase, mitochondrial | | −0.21 |
| inorganic diphosphatase | PPA2 | −0.21 |
| diphopshate transporter, mitochondrial | | −0.21 |
| 6-glutamyl-10FTHF transport, mitochondrial | | −0.21 |
| 7-glutamyl-10FTHF transport, mitochondrial | | −0.21 |
| 6-glutamyl-DHF transport, m mitochondrial | | −0.21 |
| 6-glutamyl-THF transport, m mitochondrial | | −0.21 |
| 7-glutamyl-DHF transport, m mitochondrial | | −0.21 |
| 7-glutamyl-THF transport, m mitochondrial | | −0.21 |
| folylpolyglutamate synthetase, mitochondrial | FPGS | −0.21 |
| folylpolyglutamate synthetase, mitochondrial | FPGS | −0.21 |
| folylpolyglutamate synthetase (DHF), | FPGS | −0.21 |
| mitochondrial | | |
| folylpolyglutamate synthetase (DHF), | FPGS | −0.21 |
| mitochondrial | | |
| folylpolyglutamate synthetase (DHF), | FPGS | −0.21 |
| mitochondrial | | |
| folylpolyglutamate synthetase (10fthf), | FPGS | −0.21 |
| mitochondrial | | |
| folylpolyglutamate synthetase (10fthf), | FPGS | −0.21 |
| mitochondrial | | |
| cholesterol 25-hydroxylase | CH25H | −0.21 |
| Cholest-5-ene-3beta,7alpha-diol: NAD+ 3- | HSD3B7 | −0.21 |
| oxidoreductase Bile acid biosynthesis | | |
| EC: 1.1.1.181 | | |
| superoxide anion transport via diffusion | | −0.21 |
| (mitochondria) | | |
| Glutathione transport into mitochondria | | −0.21 |
| ribulose 5-phosphate 3-epimerase | RPE | −0.21 |
| alpha-ketoglutarate/malate transporter | SLC25A10; | −0.21 |
| | SLC25A11 | |
| Mitochondrial Carrier (MC) TCDB: 2.A.29.2.7 | SLC25A10 | −0.21 |
| Mitochondrial Carrier (MC) TCDB: 2.A.29.2.7 | SLC25A10 | −0.21 |
| Mitochondrial Carrier (MC) TCDB: 2.A.29.2.1 | SLC25A11 | −0.21 |
| Mitochondrial Carrier (MC) TCDB: 2.A.29.2.1 | SLC25A11 | −0.21 |
| fatty acid retinol exchange | | −0.21 |
| Metanephrine exchange | | −0.21 |
| metanephrine secretion via secretory vesicle | | −0.21 |
| (ATP driven) | | |
| S-Adenosyl-L-methionine: catechol O- | COMTD1 | −0.21 |
| methyltransferase | | |
| Mitochondrial Carrier (MC) TCDB: 2.A.29.7.2 | SLC25A1 | −0.21 |
| 3,4-Dihydroxyphenylethyleneglycol: NAD+ | ADH1; ADH4; | −0.21 |
| oxidoreductase | ADH5; ADH6A; | |
| | ADH7; ADHFE1 | |
| 3,4-Dihydroxyphenylethyleneglycol | | −0.21 |
| transport (diffusion) | | |
| 4-[(1R)-2-Amino-1-hydroxyethyl]-1,2- | MAOA; MAOB | −0.21 |
| benzenediol: oxygen | | |
| oxidoreductase(deaminating)(flavin- | | |
| containing) | | |
| 3,4-Dihydroxyphenylethyleneglycol | | −0.21 |
| exchange | | |
| ADPribose 2′-phosphate exchange | | −0.22 |
| ADPribose exchange | | −0.22 |
| Nicotinamide adenine dinucleotide exchange | | −0.22 |
| Nicotinamide adenine dinucleotide | | −0.22 |
| phosphate exchange | | |
| NAD nucleosidase, extracellular | CD38 | −0.22 |
| NADP nucleosidase, extracellular | CD38 | −0.22 |
| dTTP nucleotidohydrolase Pyrimidine | ENTPD1; ENTPD3; | −0.22 |
| metabolism EC: 3.6.1.39 | ENTPD8 | |
| Facilitated diffusion | | −0.22 |
| ITP diphosphohydrolase Purine metabolism | ENTPD1; ENTPD3; | −0.22 |
| EC: 3.6.1.5 | ENTPD8 | |
| IDP exchange | | −0.22 |
| Exchange of 7,8-dihydroneopterin 3- | | −0.22 |
| triphosphate(4-) | | |
| CDP(3-) exchange | | −0.22 |
| CDP(3-) exchange | | −0.22 |
| CMP exchange | | −0.22 |
| ({[(2R,3S,5R)-3-hydroxy-5-(5-methyl-2,4- | | −0.22 |
| dioxo-1,2,3,4-tetrahydropyrimidin-1- | | |
| yl)oxolan-2-yl]methyl | | |
| phosphonato)oxy)phosphonate exchange | | |
| ({[(2R,3S,5R)-3-hydroxy-5-(5-methyl-2,4- | | −0.22 |
| dioxo-1,2,3,4-tetrahydropyrimidin-1- | | |
| yl)oxolan-2-yl]methyl | | |
| phosphonato)oxy)phosphonate exchange | | |
| Exchange of dTMP(2-) | | −0.22 |
| Exchange of Dihydroneopterin | | −0.22 |
| CDP diphosphohydrolase Pyrimidine | ENTPD1; ENTPD3; | −0.22 |
| metabolism EC: 3.6.1.5 | ENTPD8 | |
| dTDP diphosphohydrolase Pyrimidine | ENTPD1; ENTPD3; | −0.22 |
| metabolism EC: 3.6.1.5 | ENTPD8 | |
| 2-Amino-4-hydroxy-6-(erythro-1,2,3- | ALPL | −0.22 |
| trihydroxypropyl) dihydropteridine | | |
| triphosphate phosphohydrolase (alkaline | | |
| optimum) Folate biosynthesis EC: 3.1.3.1 | | |
| CTP diphosphohydrolase Pyrimidine | ENTPD1; ENTPD3; | −0.22 |
| metabolism EC: 3.6.1.5 | ENTPD8 | |
| Exchange of CTP(4-) | | −0.22 |
| Exchange of ITP(3-) | | −0.22 |
| Exchange of dTTP(4-) | | −0.22 |
| fumarase | FH1 | −0.22 |
| fumarase | FH1 | −0.22 |
| fumarase, mitochondrial | FH1 | −0.22 |
| fumarase, mitochondrial | FH1 | −0.22 |
| H2O transport, mitochondrial | AQP8 | −0.22 |
| H2O transport, mitochondrial | AQP8 | −0.22 |
| Transport of S-adenosyl-L-homocysteine | | −0.22 |
| Exchange of S-adenosyl-L-homocysteine | | −0.22 |
| glutathione oxidoreductase | GSR | −0.23 |
| glutathione: NAD+ oxidoreductase Glutamate | GSR | −0.23 |
| metabolism EC: 1.8.1.7 | | |
| CO2 transport (diffusion), mitochondrial | | −0.23 |
| Uracil exchange | | −0.23 |
| uracil transport via facilated diffusion | SLC29A2 | −0.23 |
| monoacylglycerol 2 transport | | −0.23 |
| phosphoribosylaminoimidazolecarboxamide | ATIC | −0.23 |
| formyltransferase | | |
| Transport of 5-amino-1-(5-phospho-D- | | −0.23 |
| ribosyl)imidazole-4-carboxamide(2-) | | |
| Exchange of 5-amino-1-(5-phospho-D- | | −0.23 |
| ribosyl)imidazole-4-carboxamide(2-) | | |
| IMP cyclohydrolase | ATIC | −0.23 |
| Glutathione: cystine oxidoreductase | | −0.23 |
| NADPH: CoA-glutathione oxidoreductase | TXNRD1; TXNRD2 | −0.23 |
| EC: 1.8.1.9 | | |
| Mitochondrial Carrier (MC) TCDB: 2.A.29.19.1 | SLC25A2 | −0.24 |
| Transport reaction | | −0.24 |
| Transport reaction | | −0.24 |
| Mitochondrial Carrier (MC) TCDB: 2.A.29.7.2 | SLC25A1 | −0.24 |
| Mitochondrial Carrier (MC) TCDB: 2.A.29.7.2 | SLC25A1 | −0.24 |
| Mitochondrial Carrier (MC) TCDB: 2.A.29.7.2 | SLC25A1 | −0.24 |
| Mitochondrial Carrier (MC) TCDB: 2.A.29.7.2 | SLC25A1 | −0.24 |
| Mitochondrial Carrier (MC) TCDB: 2.A.29.7.2 | SLC25A1 | −0.24 |
| Mitochondrial Carrier (MC) TCDB: 2.A.29.7.2 | SLC25A1 | −0.24 |
| Mitochondrial Carrier (MC) TCDB: 2.A.29.7.2 | SLC25A1 | −0.24 |
| Mitochondrial Carrier (MC) TCDB: 2.A.29.7.2 | SLC25A1 | −0.24 |
| Mitochondrial Carrier (MC) TCDB: 2.A.29.7.2 | SLC25A1 | −0.24 |
| Mitochondrial Carrier (MC) TCDB: 2.A.29.7.2 | SLC25A1 | −0.24 |
| Mitochondrial Carrier (MC) TCDB: 2.A.29.7.2 | SLC25A1 | −0.24 |
| Mitochondrial Carrier (MC) TCDB: 2.A.29.19.1 | SLC25A2 | −0.24 |
| Mitochondrial Carrier (MC) TCDB: 2.A.29.19.1 | SLC25A2 | −0.24 |
| Mitochondrial Carrier (MC) TCDB: 2.A.29.19.1 | SLC25A2 | −0.24 |
| Mitochondrial Carrier (MC) TCDB: 2.A.29.19.1 | SLC25A2 | −0.24 |
| 1-acylglycerol-3-phosphate O- | AGPAT1; AGPAT2; | −0.24 |
| acyltransferase 1 | AGPAT3; AGPAT4; | |
| | AGPAT5; GPAT4; | |
| | MBOAT2 | |
| glycerol-3-phosphate acyltransferase | GPAM; MBOAT2 | −0.24 |
| 5-glutamyl-10FTHF transport, mitochondrial | | −0.24 |
| 5-glutamyl-THF transport, m mitochondrial | | −0.24 |
| folylpolyglutamate synthetase (10fthf), | FPGS | −0.24 |
| mitochondrial | | |
| folylpolyglutamate synthetase, mitochondrial | FPGS | −0.24 |
| transport of ubiquinol into cytosol | | −0.24 |
| transport of ubiquinone into mitochondria | | −0.24 |
| Mitochondrial Carrier (MC) TCDB: 2.A.29.7.2 | SLC25A1 | −0.24 |
| adenosine facilated transport in | SLC29A1 | −0.25 |
| mitochondria | | |
| glycine hydroxymethyltransferase, reversible | SHMT1 | −0.25 |
| Mitochondrial Carrier (MC) TCDB: 2.A.29.2.2 | SLC25A10 | −0.25 |
| alpha-ketoglutarate/malate transporter | SLC25A10; | −0.25 |
| | SLC25A11 | |
| malate transport, mitochondrial | SLC25A10 | −0.25 |
| Mitochondrial Carrier (MC) TCDB: 2.A.29.2.7 | SLC25A10 | −0.25 |
| malate transport, mitochondrial | SLC25A10 | −0.25 |
| Mitochondrial Carrier (MC) TCDB: 2.A.29.2.2 | SLC25A10 | −0.25 |
| Mitochondrial Carrier (MC) TCDB: 2.A.29.2.2 | SLC25A10 | −0.25 |
| Mitochondrial Carrier (MC) TCDB: 2.A.29.19.1 | SLC25A2 | −0.25 |
| Mitochondrial Carrier (MC) TCDB: 2.A.29.19.1 | SLC25A2 | −0.25 |
| Mitochondrial Carrier (MC) TCDB: 2.A.29.19.1 | SLC25A2 | −0.25 |
| Mitochondrial Carrier (MC) TCDB: 2.A.29.19.1 | SLC25A2 | −0.25 |
| Mitochondrial Carrier (MC) TCDB: 2.A.29.19.1 | SLC25A2 | −0.25 |
| 3-beta-hydroxysteroid-delta(8),delta(7)- | EBP | −0.25 |
| isomerase | | |
| 5alpha-Cholest-7-en-3beta-ol delta7-delta8- | EBP | −0.25 |
| isomerase Biosynthesis of steroids EC: 5.3.3.5 | | |
| Exchange of formaldehyde | | −0.25 |
| Free diffusion | | −0.25 |
| Glutathione transport into mitochondria | | −0.26 |
| glutathione: NAD+ oxidoreductase Glutamate | GSR | −0.26 |
| metabolism EC: 1.8.1.7 | | |
| glutathione oxidoreductase | GSR | −0.26 |
| succinate transport, mitochondrial | SLC25A10 | −0.27 |
| Malate: sulfite antiport, mitochondrial | SLC25A10 | −0.27 |
| Mitochondrial Carrier (MC) TCDB: 2.A.29.2.2 | SLC25A10 | −0.27 |
| Mitochondrial Carrier (MC) TCDB: 2.A.29.2.2 | SLC25A10 | −0.27 |
| Mitochondrial Carrier (MC) TCDB: 2.A.29.2.2 | SLC25A10 | −0.27 |
| Mitochondrial Carrier (MC) TCDB: 2.A.29.2.2 | SLC25A10 | −0.27 |
| Mitochondrial Carrier (MC) TCDB: 2.A.29.2.2 | SLC25A10 | −0.27 |
| Mitochondrial Carrier (MC) TCDB: 2.A.29.2.7 | SLC25A10 | −0.27 |
| fumarate transport, mitochondrial | SLC25A10 | −0.27 |
| Malate: thiosulfate antiport, mitochondrial | SLC25A10 | −0.27 |
| Malate: sulfate antiport, mitochondrial | SLC25A10 | −0.27 |
| Mitochondrial Carrier (MC) TCDB: 2.A.29.2.2 | SLC25A10 | −0.27 |
| Mitochondrial Carrier (MC) TCDB: 2.A.29.2.2 | SLC25A10 | −0.27 |
| Fumarate: sulfate antiport, mitochondrial | SLC25A10 | −0.27 |
| Mitochondrial Carrier (MC) TCDB: 2.A.29.2.2 | SLC25A10 | −0.27 |
| Fumarate: thiosulfate antiport, mitochondrial | SLC25A10 | −0.27 |
| succinate transport, mitochondrial | SLC25A10 | −0.27 |
| Malate: sulfite antiport, mitochondrial | SLC25A10 | −0.27 |
| Fumarate: sulfite antiport, mitochondrial | SLC25A10 | −0.27 |
| fumarate transport, mitochondrial | SLC25A10 | −0.27 |
| Malate: thiosulfate antiport, mitochondrial | SLC25A10 | −0.27 |
| Fumarate: sulfite antiport, mitochondrial | SLC25A10 | −0.27 |
| Mitochondrial Carrier (MC) TCDB: 2.A.29.7.2 | SLC25A1 | −0.27 |
| Mitochondrial Carrier (MC) TCDB: 2.A.29.2.2 | SLC25A10 | −0.27 |
| Fumarate: sulfate antiport, mitochondrial | SLC25A10 | −0.27 |
| Fumarate: thiosulfate antiport, mitochondrial | SLC25A10 | −0.27 |
| Mitochondrial Carrier (MC) TCDB: 2.A.29.2.2 | SLC25A10 | −0.27 |
| Mitochondrial Carrier (MC) TCDB: 2.A.29.2.2 | SLC25A10 | −0.27 |
| Malate: sulfate antiport, mitochondrial | SLC25A10 | −0.27 |
| Mitochondrial Carrier (MC) TCDB: 2.A.29.2.2 | SLC25A10 | −0.27 |
| Mitochondrial Carrier (MC) TCDB: 2.A.29.2.2 | SLC25A10 | −0.27 |
| Mitochondrial Carrier (MC) TCDB: 2.A.29.2.7 | SLC25A10 | −0.27 |
| methanol exchange | | −0.27 |
| Methanol diffusion | | −0.27 |
| 4-[(1R)-1-Hydroxy-2-(methylamino)ethyl]- | MAOA; MAOB | −0.27 |
| 1,2-benzenediol: oxygen | | |
| oxidoreductase(deaminating)(flavin- | | |
| containing) | | |
| Methylamine oxidase | AOC1; AOC2; | −0.27 |
| | AOC3 | |
| monoacylglycerol 2 transport | | −0.28 |
| lipase | MGLL | −0.28 |

Table 2. Top ranking positive (Table 2A) and negative (Table 2B) correlating pathways from Table 1.

TABLE 2A

| | | | | genes_{—} | patho_manual_{—} |
|---|---|---|---|---|---|
| | | | rxn_EC_{—} | associated_{—} | c.cellPaper_{—} |
| rxn | rxn_name_long | subsystem | number | with_rxn | Th17n_48 hr |
| AKR1C41_pos | aldo-keto reductase family 1, | Bile acid | 1.1.1.50 | AKR1C6 | 0.32 |
| | member C4 (chlordecone | synthesis | | | |
| | reductase; 3-alpha | | | | |
| | hydroxysteroid | | | | |
| | dehydrogenase, type I; | | | | |
| | dihydrodiol dehydrogenase 4) | | | | |
| AKR1C42_pos | aldo-keto reductase family 1, | Bile acid | 1.1.1.50 | AKR1C6 | 0.32 |
| | member C4 (chlordecone | synthesis | | | |
| | reductase; 3-alpha | | | | |
| | hydroxysteroid | | | | |
| | dehydrogenase, type I; | | | | |
| | dihydrodiol dehydrogenase 4) | | | | |
| r0747_pos | 3alpha,7alpha-Dihydroxy- | Bile acid | 1.1.1.50 | AKR1C6 | 0.32 |
| | 5beta-cholestane: NADP+ | synthesis | | | |
| | oxidoreductase (B-specific); | | | | |
| | 3alpha,7alpha-Dihydroxy- | | | | |
| | 5beta-cholestane: NADP+ | | | | |
| | oxidoreductase Bile acid | | | | |
| | biosynthesis EC: 1.1.1.50 | | | | |
| r0750_pos | 3alpha,7alpha,12alpha- | Bile acid | 1.1.1.50 | AKR1C6 | 0.32 |
| | Trihydroxy-5beta- | synthesis | | | |
| | cholestane: NADP+ | | | | |
| | oxidoreductase (B-specific); | | | | |
| | 3alpha,7alpha,12alpha- | | | | |
| | Trihydroxy-5beta- | | | | |
| | cholestane: NADP+ | | | | |
| | oxidoreductase Bile acid | | | | |
| | biosynthesis EC: 1.1.1.50 | | | | |
| DURAD2_pos | dihydrothymin | Pyrimidine | 1.3.1.2 | DPYD | 0.32 |
| | dehydrogenase (NADP) | catabolism | | | |
| r0330_pos | 5,6-Dihydrothymine: NAD+ | Pyrimidine | 1.3.1.1 | | 0.32 |
| | oxidoreductase Pyrimidine | catabolism | | | |
| | metabolism EC: 1.3.1.1 | | | | |
| r0267_pos | CMP-N- | Unassigned | 1.14.18.2 | | 0.32 |
| | acetylneuraminate, | | | | |
| | ferrocytochrome-b5: oxygen | | | | |
| | oxidoreductase (N-acetyl- | | | | |
| | hydroxylating) Aminosugars | | | | |
| | metabolism EC: 1.14.18.2 | | | | |
| r0268_neg | cytidine monophospho-N- | Unassigned | 1.14.18.2 | | 0.32 |
| | acetylneuraminic acid | | | | |
| | hydroxylase EC: 1.14.18.2 | | | | |
| G6PDH2r_neg | glucose 6-phosphate | Pentose | 1.1.1.49 | G6PD2 | 0.31 |
| | dehydrogenase | phosphate | | | |
| | | pathway | | | |
| PYK_pos | pyruvate kinase | Glycolysis/ | N/A | PKLR; PKM | 0.30 |
| | | gluconeogenesis | | | |
| NDPK4_pos | nucleoside-diphosphate | Nucleotide | N/A | GM20390; | 0.30 |
| | kinase (ATP: dTDP) | interconversion | | NME2; | |
| | | | | NME3; | |
| | | | | NME6; | |
| | | | | NME7 | |
| RE2954C_neg | RE2954 | Pyrimidine | 2.7.1.40 | PKM | 0.30 |
| | | synthesis | | | |
| G6PDH2r_pos | glucose 6-phosphate | Pentose | 1.1.1.49 | G6PD2 | 0.29 |
| | dehydrogenase | phosphate | | | |
| | | pathway | | | |
| FBA_pos | fructose-bisphosphate | Glycolysis/ | 4.1.2.13 | ALDOART2; | 0.27 |
| | aldolase | gluconeogenesis | | ALDOB; | |
| | | | | ALDOC | |
| PFK_pos | phosphofructokinase | Glycolysis/ | N/A | PFKL; | 0.27 |
| | | gluconeogenesis | | PFKM; | |
| | | | | PFKP | |
| TALA_pos | transaldolase | Pentose | 2.2.1.2 | TALDO1 | 0.27 |
| | | phosphate | | | |
| | | pathway | | | |
| r0407_neg | Sedoheptulose 1,7- | Unassigned | 4.1.2.13 | ALDOART2; | 0.27 |
| | bisphosphate D- | | | ALDOB; | |
| | glyceraldehyde-3-phosphate- | | | ALDOC | |
| | lyase Carbon fixation | | | | |
| | EC: 4.1.2.13 | | | | |
| r0191_pos | UTP: D-fructose-6-phosphate | Fructose | 2.7.1.11 | PFKL | 0.26 |
| | 1-phosphotransferase | and | | | |
| | EC: 2.7.1.11 | mannose | | | |
| | | metabolism | | | |
| ALCD22_D_pos | alcohol dehydrogenase (D- | Pyruvate | 1.1.1.78 | ADH1; | 0.26 |
| | lactaldehyde) | metabolism | | ADH4; | |
| | | | | ADH5; | |
| | | | | ADH6A; | |
| | | | | ADH7; | |
| | | | | ADHFE1; | |
| | | | | ZADH2 | |
| r0408_neg | ATP: Sedoheptulose 7- | Unassigned | 2.7.1.11 | PFKL | 0.25 |
| | phosphate 1- | | | | |
| | phosphotransferase | | | | |
| | EC: 2.7.1.11 | | | | |
| r0409_neg | UTP: Sedoheptulose 7- | Unassigned | 2.7.1.11 | PFKL | 0.25 |
| | phosphate 1- | | | | |
| | phosphotransferase | | | | |
| | EC: 2.7.1.11 | | | | |
| NDPK9_neg | nucleoside-diphosphate | Nucleotide | 2.7.4.6 | GM20390; | 0.25 |
| | kinase (ATP: IDP) | interconversion | | NME2; | |
| | | | | NME3; | |
| | | | | NME6; | |
| | | | | NME7 | |
| NDPK9_pos | nucleoside-diphosphate | Nucleotide | 2.7.4.6 | GM20390; | 0.25 |
| | kinase (ATP: IDP) | interconversion | | NME2; | |
| | | | | NME3; | |
| | | | | NME6; | |
| | | | | NME7 | |
| r0408_pos | ATP: Sedoheptulose 7- | Unassigned | 2.7.1.11 | PFKL | 0.25 |
| | phosphate 1- | | | | |
| | phosphotransferase | | | | |
| | EC: 2.7.1.11 | | | | |
| r0409_pos | UTP: Sedoheptulose 7- | Unassigned | 2.7.1.11 | PFKL | 0.25 |
| | phosphate 1- | | | | |
| | phosphotransferase | | | | |
| | EC: 2.7.1.11 | | | | |
| r0610_neg | CTP: D-Tagatose 6-phosphate | Unassigned | 2.7.1.11 | PFKL | 0.25 |
| | 1-phosphotransferase | | | | |
| | Galactose metabolism | | | | |
| | EC: 2.7.1.11 | | | | |
| r0610_pos | CTP: D-Tagatose 6-phosphate | Unassigned | 2.7.1.11 | PFKL | 0.25 |
| | 1-phosphotransferase | | | | |
| | Galactose metabolism | | | | |
| | EC: 2.7.1.11 | | | | |
| r0611_neg | ITP: D-Tagatose 6-phosphate | Unassigned | 2.7.1.11 | PFKL | 0.25 |
| | 1-phosphotransferase | | | | |
| | Galactose metabolism | | | | |
| | EC: 2.7.1.11 | | | | |
| r0611_pos | ITP: D-Tagatose 6-phosphate | Unassigned | 2.7.1.11 | PFKL | 0.25 |
| | 1-phosphotransferase | | | | |
| | Galactose metabolism | | | | |
| | EC: 2.7.1.11 | | | | |
| EX_mthgxl(e)_pos | Methylglyoxal exchange | Exchange/ | N/A | | 0.25 |
| | | demand | | | |
| | | reaction | | | |
| MTHGXLt_pos | Methylglyoxal transport | Transport, | N/A | | 0.25 |
| | (cytosol to extracellular) | extracellular | | | |
| RDH1_neg | retinol dehydrogenase (all- | Vitamin A | 1.1.1.105 | RDH16F1; | 0.24 |
| | trans) | metabolism | | RDH5 | |
| RDH1a_pos | retinol dehydrogenase (all- | Vitamin A | 1.1.1.105 | RDH10; | 0.24 |
| | trans, NADPH) | metabolism | | RDH11; | |
| | | | | RDH12; | |
| | | | | RDH13; | |
| | | | | RDH14; | |
| | | | | RDH8; | |
| | | | | SDR16C5 | |
| RDH2_neg | retinol dehydrogenase (9- | Vitamin A | 1.1.1.105 | RDH5 | 0.24 |
| | cis, NADH) | metabolism | | | |
| RDH2a_pos | retinol dehydrogenase (9- | Vitamin A | 1.1.1.105 | RDH11; | 0.24 |
| | cis, NADPH) | metabolism | | RDH12; | |
| | | | | RDH13; | |
| | | | | RDH14; | |
| | | | | RDH8; | |
| | | | | SDR16C5 | |
| RDH3_neg | retinol dehydrogenase (11- | Vitamin A | 1.1.1.105 | RDH5 | 0.24 |
| | cis, NADH) | metabolism | | | |
| RDH3a_pos | retinol dehydrogenase (11- | Vitamin A | 1.1.1.105 | RDH11; | 0.24 |
| | cis, NADPH) | metabolism | | RDH12; | |
| | | | | RDH13; | |
| | | | | RDH14; | |
| | | | | SDR16C5 | |
| RETI2_neg | retinol isomerase (9-cis) | Vitamin A | 5.2.1.7 | | 0.24 |
| | | metabolism | | | |
| RETI1_neg | retinol isomerase (11-cis) | Vitamin A | 5.2.1.7 | | 0.24 |
| | | metabolism | | | |
| RETI1_pos | retinol isomerase (11-cis) | Vitamin A | 5.2.1.7 | | 0.24 |
| | | metabolism | | | |
| RETI2_pos | retinol isomerase (9-cis) | Vitamin A | 5.2.1.7 | | 0.24 |
| | | metabolism | | | |
| RAI1_neg | retinal isomerase (11-cis) | Vitamin A | 5.2.1.3 | | 0.24 |
| | | metabolism | | | |
| RAI1_pos | retinal isomerase (11-cis) | Vitamin A | 5.2.1.3 | | 0.24 |
| | | metabolism | | | |
| RAI2_pos | retinal isomerase (9-cis) | Vitamin A | 5.2.1.3 | | 0.24 |
| | | metabolism | | | |
| RAI2_neg | retinal isomerase (9-cis) | Vitamin A | 5.2.1.3 | | 0.24 |
| | | metabolism | | | |
| FALDH_neg | formaldehyde | Tyrosine | 1.2.1.1 | ADH5 | 0.24 |
| | dehydrogenase | metabolism | | | |
| FALDH_pos | formaldehyde | Tyrosine | 1.2.1.1 | ADH5 | 0.24 |
| | dehydrogenase | metabolism | | | |
| r1377_neg | S- | Unassigned | 4.4.1.22 | | 0.24 |
| | (hydroxymethyl)glutathione | | | | |
| | synthase Methane | | | | |
| | metabolism EC: 4.4.1.22 | | | | |
| r1377_pos | S- | Unassigned | 4.4.1.22 | | 0.24 |
| | (hydroxymethyl)glutathione | | | | |
| | synthase Methane | | | | |
| | metabolism EC: 4.4.1.22 | | | | |
| r1378_neg | S- | Unassigned | 1.1.1.284 | ADH5 | 0.24 |
| | (hydroxymethyl)glutathione | | | | |
| | dehydrogenase Methane | | | | |
| | metabolism EC: 1.1.1.284 | | | | |
| r1378_pos | S- | Unassigned | 1.1.1.284 | ADH5 | 0.24 |
| | (hydroxymethyl)glutathione | | | | |
| | dehydrogenase Methane | | | | |
| | metabolism EC: 1.1.1.284 | | | | |
| EX_bilirub(e)_neg | Bilirubin exchange | Exchange/ | N/A | | 0.24 |
| | | demand | | | |
| | | reaction | | | |
| NTD3_pos | 5′-nucleotidase (dCMP) | Pyrimidine | 3.1.3.5 | GUCA1A; | 0.24 |
| | | catabolism | | NT5C; | |
| | | | | NT5C1A; | |
| | | | | NT5C1B; | |
| | | | | NT5C3; | |
| | | | | NT5E | |
| r0377_pos | ATP: deoxycitidine 5- | Pyrimidine | 2.7.1.74 | DCK | 0.24 |
| | phosphotransferase | catabolism | | | |
| | Pyrimidine metabolism | | | | |
| | EC: 2.7.1.74 | | | | |
| r2197_pos | Resistance-Nodulation-Cell | Transport, | N/A | SLCO1A1 | 0.23 |
| | Division (RND) | extracellular | | | |
| | TCDB: 2.A.60.1.14 | | | | |
| r2141_pos | Resistance-Nodulation-Cell | Transport, | N/A | SLCO1A1 | 0.23 |
| | Division (RND) | extracellular | | | |
| | TCDB: 2.A.60.1.14 | | | | |
| r2194_pos | Resistance-Nodulation-Cell | Transport, | N/A | SLCO1A1 | 0.23 |
| | Division (RND) | extracellular | | | |
| | TCDB: 2.A.60.1.14 | | | | |
| r2195_pos | Resistance-Nodulation-Cell | Transport, | N/A | SLCO1A1 | 0.23 |
| | Division (RND) | extracellular | | | |
| | TCDB: 2.A.60.1.14 | | | | |
| TCHOLAt3_pos | ABC bile acid transporter | Transport, | N/A | ABCB11; | 0.23 |
| | | extracellular | | ABCC3 | |
| PAIL4P_HStn_pos | phosphatidylinositol 4- | Transport, | N/A | | 0.23 |
| | phosphate nuclear transport | nuclear | | | |
| | (diffusion) | | | | |
| H2Otn_pos | H2O transport, nuclear | Transport, | N/A | | 0.23 |
| | | nuclear | | | |
| PAIL45P_HStn_neg | phosphatidylinositol 4,5- | Transport, | N/A | | 0.23 |
| | bisphosphate nuclear | nuclear | | | |
| | transport (diffusion) | | | | |
| PItn_neg | phosphate transport, nuclear | Transport, | N/A | | 0.23 |
| | | nuclear | | | |
| r1017_pos | Vesicular transport | Transport, | N/A | | 0.22 |
| | | extracellular | | | |
| TCHOLAtx_neg | bile acid intracellular | Transport, | N/A | | 0.22 |
| | transport | peroxisomal | | | |
| GND_pos | phosphogluconate | Pentose | 1.1.1.44 | PGD | 0.22 |
| | dehydrogenase | phosphate | | | |
| | | pathway | | | |
| PI4P5Kn_pos | phosphatidylinositol 4- | Inositol | 2.7.1.68 | | 0.22 |
| | phosphate 5-kinase, nuclear | phosphate | | | |
| | | metabolism | | | |
| DCK1n_neg | Deoxycytidine kinase, nuclear | Nucleotide | 2.7.1.74 | DCK | 0.22 |
| | (ATP) | interconversion | | | |
| DCK1n_pos | Deoxycytidine kinase, nuclear | Nucleotide | 2.7.1.74 | DCK | 0.22 |
| | (ATP) | interconversion | | | |
| DCK2n_neg | Deoxycytidine kinase, nuclear | Nucleotide | 2.7.1.74 | DCK | 0.22 |
| | (UTP) | interconversion | | | |
| DCK2n_pos | Deoxycytidine kinase, nuclear | Nucleotide | 2.7.1.74 | DCK | 0.22 |
| | (UTP) | interconversion | | | |
| EX_retnglc(e)_pos | retinoyl glucuronide | Exchange/ | N/A | | 0.21 |
| | exchange | demand | | | |
| | | reaction | | | |
| RETNGLCt2r_pos | retinoyl glucuronide efflux | Transport, | N/A | | 0.21 |
| | (13-cis) from ER | endoplasmic | | | |
| | | reticular | | | |
| RETNGLCtr_pos | retinoyl glucuronide efflux | Transport, | N/A | | 0.21 |
| | from ER | endoplasmic | | | |
| | | reticular | | | |
| RETNtr_pos | retinoic acid transport in ER | Transport, | N/A | | 0.21 |
| | | endoplasmic | | | |
| | | reticular | | | |
| RETNtr2_pos | retinoic acid transport in ER | Transport, | N/A | | 0.21 |
| | (13-cis) | endoplasmic | | | |
| | | reticular | | | |
| RETNGLCt_pos | retinoyl glucuronide efflux | Transport, | N/A | | 0.21 |
| | | extracellular | | | |
| UGT1A5r_pos | UDP-glucuronosyltransferase | Vitamin A | 2.4.1.17 | UGT1A8 | 0.21 |
| | 1-10 precursor, microsomal | metabolism | | | |
| UGT1A5r2_pos | UDP-glucuronosyltransferase | Vitamin A | 2.4.1.17 | UGT1A1; | 0.21 |
| | 1-10 precursor, microsomal | metabolism | | UGT1A2; | |
| | (13-cis) | | | UGT1A7C; | |
| | | | | UGT1A8; | |
| | | | | UGT2B34 | |
| EX_glc(e)_pos | D-Glucose exchange | Exchange/ | N/A | | 0.21 |
| | | demand | | | |
| | | reaction | | | |
| PI5P4Kn_pos | phosphatidylinositol-5- | Inositol | 2.7.1.149 | | 0.21 |
| | phosphate 4-kinase, nuclear | phosphate | | | |
| | | metabolism | | | |
| PIK5n_pos | phosphatidylinositol 5-kinase, | Inositol | N/A | | 0.21 |
| | nuclear | phosphate | | | |
| | | metabolism | | | |
| PGL_pos | 6-phosphogluconolactonase | Pentose | 3.1.1.31 | PGLS | 0.20 |
| | | phosphate | | | |
| | | pathway | | | |
| TCHOLAt_pos | taurocholate transport via | Transport, | N/A | SLCO1A1; | 0.20 |
| | bicarbonate countertransport | extracellular | | SLCO1B2; | |
| | | | | SLCO4A1 | |
| THYOXt_pos | T4 transport via bicarbonate | Transport, | N/A | SLCO1A1; | 0.20 |
| | countertransport | extracellular | | SLCO1B2; | |
| | | | | SLCO1C1; | |
| | | | | SLCO4A1 | |
| THYOXt2_neg | T4 transport via facilitated | Transport, | N/A | SLC16A2 | 0.20 |
| | diffusion | extracellular | | | |
| TRIODTHYt_pos | T3 transport via bicarbonate | Transport, | N/A | SLCO1A1; | 0.20 |
| | countertransport | extracellular | | SLCO1B2; | |
| | | | | SLCO1C1; | |
| | | | | SLCO4A1 | |
| TRIODTHYt2_neg | T3 transport via facilitated | Transport, | N/A | SLC16A2 | 0.20 |
| | diffusion | extracellular | | | |
| TCHOLAte_neg | bile acid intracellular | Transport, | N/A | | 0.20 |
| | transport | extracellular | | | |
| DHFR_neg | dihydrofolate reductase | Folate | 1.5.1.3 | DHFR | 0.20 |
| | | metabolism | | | |
| r0224_pos | 5,6,7,8- | Folate | 1.5.1.3 | DHFR | 0.20 |
| | Tetrahydrofolate: NAD+ | metabolism | | | |
| | oxidoreductase One carbon | | | | |
| | pool by folate EC: 1.5.1.3 | | | | |
| EX_prostge2(e)_neg | Prostaglandin E2 exchange | Exchange/ | N/A | | 0.20 |
| | | demand | | | |
| | | reaction | | | |
| GALt2_2_pos | D-galactose transport via | Transport, | N/A | SLC5A1 | 0.20 |
| | proton symport | extracellular | | | |
| GLCt2_2_pos | D-glucose transport in via | Transport, | N/A | SLC5A1 | 0.20 |
| | proton symport | extracellular | | | |
| LALDO_pos | D-Lactaldehyde: NAD+ | Pyruvate | 1.1.1.1 | ADH5 | 0.20 |
| | oxidoreductase (glutathione- | metabolism | | | |
| | formylating) | | | | |
| RE2404C_pos | RE2404 | Miscellaneous | 2.4.1.17 | UGT1A1 | 0.19 |
| RE2405C_pos | RE2405 | Miscellaneous | 2.4.1.17 | UGT1A1 | 0.19 |
| RE2541C_pos | RE2541 | Miscellaneous | 3.2.1.31 | KL | 0.19 |
| RE3381C_pos | RE3381 | Miscellaneous | 3.2.1.31 | KL | 0.19 |

| | patho_manual_{—} | patho_manual_{—} | patho_manual_c.only_{—} | patho_manual_c.only_{—} |
|---|---|---|---|---|
| | c.cellPaper_{—} | c.cellPaper_{—} | Th17p.cellPaper_{—} | Th17p.cellPaper_{—} |
| rxn | vitro_48 hr_GFP | vivo | vitro_48 hr_GFP | vivo |
| AKR1C41_pos | −0.05 | 0.09 | −0.03 | 0.06 |
| AKR1C42_pos | −0.05 | 0.09 | −0.03 | 0.06 |
| r0747_pos | −0.05 | 0.09 | −0.03 | 0.06 |
| r0750_pos | −0.05 | 0.09 | −0.03 | 0.06 |
| DURAD2_pos | −0.05 | 0.09 | −0.03 | 0.06 |
| r0330_pos | −0.05 | 0.09 | −0.03 | 0.06 |
| r0267_pos | −0.05 | 0.09 | −0.03 | 0.06 |
| r0268_neg | −0.05 | 0.09 | −0.03 | 0.06 |
| G6PDH2r_neg | −0.05 | 0.05 | 0.01 | 0.04 |
| PYK_pos | 0.16 | 0.13 | −0.16 | 0.01 |
| NDPK4_pos | 0.16 | 0.13 | −0.16 | 0.01 |
| RE2954C_neg | 0.16 | 0.13 | −0.16 | 0.01 |
| G6PDH2r_pos | 0.08 | 0.16 | −0.07 | 0.01 |
| FBA_pos | 0.16 | 0.11 | −0.01 | 0.08 |
| PFK_pos | 0.16 | 0.11 | −0.01 | 0.08 |
| TALA_pos | 0.16 | 0.11 | −0.01 | 0.08 |
| r0407_neg | 0.16 | 0.11 | −0.01 | 0.08 |
| r0191_pos | 0.16 | 0.07 | −0.01 | 0.07 |
| ALCD22_D_pos | 0.03 | 0.14 | −0.09 | 0.16 |
| r0408_neg | 0.15 | 0.08 | −0.03 | 0.05 |
| r0409_neg | 0.15 | 0.08 | −0.03 | 0.05 |
| NDPK9_neg | 0.15 | 0.02 | −0.03 | 0.05 |
| NDPK9_pos | 0.15 | 0.02 | −0.03 | 0.05 |
| r0408_pos | 0.15 | 0.02 | −0.03 | 0.05 |
| r0409_pos | 0.15 | 0.02 | −0.03 | 0.05 |
| r0610_neg | 0.15 | 0.02 | −0.03 | 0.05 |
| r0610_pos | 0.15 | 0.02 | −0.03 | 0.05 |
| r0611_neg | 0.15 | 0.02 | −0.03 | 0.05 |
| r0611_pos | 0.15 | 0.02 | −0.03 | 0.05 |
| EX_mthgxl(e)_pos | 0.36 | 0.31 | −0.06 | 0.12 |
| MTHGXLt_pos | 0.36 | 0.31 | −0.06 | 0.12 |
| RDH1_neg | 0.14 | 0.11 | −0.05 | 0.19 |
| RDH1a_pos | 0.14 | 0.11 | −0.05 | 0.19 |
| RDH2_neg | 0.12 | 0.11 | −0.06 | 0.19 |
| RDH2a_pos | 0.12 | 0.11 | −0.06 | 0.19 |
| RDH3_neg | 0.12 | 0.11 | −0.06 | 0.19 |
| RDH3a_pos | 0.12 | 0.11 | −0.06 | 0.19 |
| RETI2_neg | 0.14 | 0.10 | −0.05 | 0.21 |
| RETI1_neg | 0.14 | 0.09 | −0.05 | 0.21 |
| RETI1_pos | 0.12 | 0.08 | −0.06 | 0.19 |
| RETI2_pos | 0.12 | 0.08 | −0.06 | 0.20 |
| RAI1_neg | 0.12 | 0.08 | −0.06 | 0.19 |
| RAI1_pos | 0.14 | 0.07 | −0.05 | 0.18 |
| RAI2_pos | 0.14 | 0.06 | −0.05 | 0.21 |
| RAI2_neg | 0.12 | 0.05 | −0.06 | 0.14 |
| FALDH_neg | 0.04 | 0.07 | −0.02 | 0.05 |
| FALDH_pos | 0.04 | 0.07 | −0.02 | 0.05 |
| r1377_neg | 0.04 | 0.07 | −0.02 | 0.05 |
| r1377_pos | 0.04 | 0.07 | −0.02 | 0.05 |
| r1378_neg | 0.04 | 0.07 | −0.02 | 0.05 |
| r1378_pos | 0.04 | 0.07 | −0.02 | 0.05 |
| EX_bilirub(e)_neg | 0.23 | 0.24 | 0.05 | 0.12 |
| NTD3_pos | 0.09 | 0.29 | 0.03 | 0.12 |
| r0377_pos | 0.09 | 0.29 | 0.03 | 0.12 |
| r2197_pos | 0.37 | −0.03 | 0.22 | 0.03 |
| r2141_pos | 0.44 | −0.03 | 0.21 | 0.03 |
| r2194_pos | 0.44 | −0.03 | 0.21 | 0.03 |
| r2195_pos | 0.44 | −0.03 | 0.21 | 0.03 |
| TCHOLAt3_pos | 0.38 | 0.31 | 0.14 | 0.15 |
| PAIL4P_HStn_pos | 0.11 | 0.27 | 0.00 | 0.09 |
| H2Otn_pos | −0.20 | −0.03 | 0.09 | −0.16 |
| PAIL45P_HStn_neg | −0.20 | −0.03 | 0.09 | −0.16 |
| PItn_neg | −0.20 | −0.03 | 0.09 | −0.16 |
| r1017_pos | 0.36 | 0.32 | 0.11 | 0.16 |
| TCHOLAtx_neg | 0.36 | 0.32 | 0.11 | 0.16 |
| GND_pos | 0.14 | 0.16 | −0.09 | −0.03 |
| PI4P5Kn_pos | 0.14 | 0.26 | −0.02 | 0.09 |
| DCK1n_neg | −0.17 | −0.01 | 0.10 | −0.04 |
| DCK1n_pos | −0.17 | −0.01 | 0.10 | −0.04 |
| DCK2n_neg | −0.17 | −0.01 | 0.10 | −0.04 |
| DCK2n_pos | −0.17 | −0.01 | 0.10 | −0.04 |
| EX_retnglc(e)_pos | 0.21 | 0.21 | −0.08 | 0.13 |
| RETNGLCt2r_pos | 0.21 | 0.21 | −0.08 | 0.13 |
| RETNGLCtr_pos | 0.21 | 0.21 | −0.08 | 0.13 |
| RETNtr_pos | 0.21 | 0.21 | −0.08 | 0.13 |
| RETNtr2_pos | 0.21 | 0.21 | −0.08 | 0.13 |
| RETNGLCt_pos | 0.21 | 0.21 | −0.08 | 0.13 |
| UGT1A5r_pos | 0.21 | 0.21 | −0.08 | 0.13 |
| UGT1A5r2_pos | 0.21 | 0.21 | −0.08 | 0.13 |
| EX_glc(e)_pos | 0.25 | 0.25 | −0.04 | 0.15 |
| PI5P4Kn_pos | 0.14 | 0.26 | −0.02 | 0.09 |
| PIK5n_pos | 0.14 | 0.26 | −0.02 | 0.09 |
| PGL_pos | 0.06 | 0.20 | −0.09 | 0.03 |
| TCHOLAt_pos | 0.43 | 0.16 | 0.23 | 0.13 |
| THYOXt_pos | 0.43 | 0.16 | 0.23 | 0.13 |
| THYOXt2_neg | 0.43 | 0.16 | 0.23 | 0.13 |
| TRIODTHYt_pos | 0.43 | 0.16 | 0.23 | 0.13 |
| TRIODTHYt2_neg | 0.43 | 0.16 | 0.23 | 0.13 |
| TCHOLAte_neg | 0.33 | 0.16 | 0.21 | 0.13 |
| DHFR_neg | −0.05 | 0.04 | 0.01 | −0.01 |
| r0224_pos | −0.05 | 0.04 | 0.01 | −0.01 |
| EX_prostge2(e)_neg | 0.14 | −0.07 | 0.16 | 0.01 |
| GALt2_2_pos | 0.12 | 0.24 | −0.07 | 0.01 |
| GLCt2_2_pos | 0.13 | 0.24 | −0.09 | 0.01 |
| LALDO_pos | 0.09 | 0.20 | −0.08 | 0.11 |
| RE2404C_pos | −0.01 | 0.26 | −0.03 | 0.16 |
| RE2405C_pos | −0.01 | 0.26 | −0.03 | 0.16 |
| RE2541C_pos | −0.01 | 0.26 | −0.03 | 0.16 |
| RE3381C_pos | −0.01 | 0.26 | −0.03 | 0.16 |

TABLE 2B

| | | | | genes_{—} | patho_manual_{—} |
|---|---|---|---|---|---|
| | | | rxn_EC_{—} | associated_{—} | c.cellPaper_{—} |
| rxn | rxn_name_long | subsystem | number | with_rxn | Th17n_48 hr |
| EX_C02470(e)_pos | Xanthurenic acid exchange | Exchange/ | N/A | | −0.36 |
| | | demand | | | |
| | | reaction | | | |
| r1030_pos | Transport reaction | Transport, | N/A | | −0.36 |
| | | extracellular | | | |
| KYN3OX_pos | kynurenine 3- | Tryptophan | 1.14.13.9 | KMO | −0.36 |
| | monooxygenase | metabolism | | | |
| RE2349C_pos | RE2349 | Tryptophan | 2.6.1.7 | KYAT1 | −0.36 |
| | | metabolism | | | |
| EX_gthrd(e)_neg | Reduced glutathione | Exchange/ | N/A | | −0.29 |
| | exchange | demand | | | |
| | | reaction | | | |
| EX_CE2011(e)_pos | hypothiocyanite exchange | Exchange/ | N/A | | −0.28 |
| | | demand | | | |
| | | reaction | | | |
| RE0702E_pos | RE0702 | Miscellaneous | 1.8.1.4 | LPO | −0.28 |
| r1725_pos | Y+LAT2 Utilized transport | Transport, | N/A | SLC7A6 | −0.25 |
| | | extracellular | | | |
| r1728_pos | Y+LAT2 Utilized transport | Transport, | N/A | SLC7A6 | −0.25 |
| | | extracellular | | | |
| r1731_pos | Y+LAT2 Utilized transport | Transport, | N/A | SLC7A6 | −0.25 |
| | | extracellular | | | |
| r1876_pos | Y+LAT2 Utilized transport | Transport, | N/A | SLC7A6 | −0.25 |
| | | extracellular | | | |
| r1879_pos | Y+LAT2 Utilized transport | Transport, | N/A | SLC7A6 | −0.25 |
| | | extracellular | | | |
| r1881_pos | Y+LAT2 Utilized transport | Transport, | N/A | SLC7A6 | −0.25 |
| | | extracellular | | | |
| r1695_pos | Y+LAT2 Utilized transport | Transport, | N/A | SLC7A6 | −0.25 |
| | | extracellular | | | |
| r1698_pos | Y+LAT2 Utilized transport | Transport, | N/A | SLC7A6 | −0.25 |
| | | extracellular | | | |
| r1701_pos | Y+LAT2 Utilized transport | Transport, | N/A | SLC7A6 | −0.25 |
| | | extracellular | | | |
| r1800_pos | Y+LAT2 Utilized transport | Transport, | N/A | SLC7A6 | −0.25 |
| | | extracellular | | | |
| r1803_pos | Y+LAT2 Utilized transport | Transport, | N/A | SLC7A6 | −0.25 |
| | | extracellular | | | |
| r1806_pos | Y+LAT2 Utilized transport | Transport, | N/A | SLC7A6 | −0.25 |
| | | extracellular | | | |
| HPYRDC_pos | hydroxypyruvate | Glyoxylate | 4.1.1.40 | | −0.25 |
| | decarboxylase | and | | | |
| | | dicarboxylate | | | |
| | | metabolism | | | |
| r1799_pos | Y+LAT2 Utilized transport | Transport, | N/A | SLC7A6 | −0.25 |
| | | extracellular | | | |
| r1722_pos | Y+LAT2 Utilized transport | Transport, | N/A | SLC7A6 | −0.25 |
| | | extracellular | | | |
| r1723_pos | Y+LAT2 Utilized transport | Transport, | N/A | SLC7A6 | −0.25 |
| | | extracellular | | | |
| r1873_pos | Y+LAT2 Utilized transport | Transport, | N/A | SLC7A6 | −0.25 |
| | | extracellular | | | |
| r1874_pos | Y+LAT2 Utilized transport | Transport, | N/A | SLC7A6 | −0.25 |
| | | extracellular | | | |
| r1798_pos | Y+LAT2 Utilized transport | Transport, | N/A | SLC7A6 | −0.25 |
| | | extracellular | | | |
| r1683_pos | Y+LAT2 Utilized transport | Transport, | N/A | SLC7A6 | −0.25 |
| | | extracellular | | | |
| r1686_pos | Y+LAT2 Utilized transport | Transport, | N/A | SLC7A6 | −0.25 |
| | | extracellular | | | |
| r1677_pos | Y+LAT2 Utilized transport | Transport, | N/A | SLC7A6 | −0.25 |
| | | extracellular | | | |
| r1721_pos | Y+LAT2 Utilized transport | Transport, | N/A | SLC7A6 | −0.25 |
| | | extracellular | | | |
| r1872_pos | Y+LAT2 Utilized transport | Transport, | N/A | SLC7A6 | −0.25 |
| | | extracellular | | | |
| r1693_pos | Y+LAT2 Utilized transport | Transport, | N/A | SLC7A6 | −0.25 |
| | | extracellular | | | |
| r1692_pos | Y+LAT2 Utilized transport | Transport, | N/A | SLC7A6 | −0.25 |
| | | extracellular | | | |
| r1797_pos | Y+LAT2 Utilized transport | Transport, | N/A | SLC7A6 | −0.25 |
| | | extracellular | | | |
| r1691_pos | Y+LAT2 Utilized transport | Transport, | N/A | SLC7A6 | −0.25 |
| | | extracellular | | | |
| EX_cyan(e)_neg | Hydrogen cyanide exchange | Exchange/ | N/A | | −0.25 |
| | | demand | | | |
| | | reaction | | | |
| MCPST_pos | 3-mercaptopyruvate | Methionine | 2.8.1.2 | MPST | −0.25 |
| | sulfurtransferase | and | | | |
| | | cysteine | | | |
| | | metabolism | | | |
| CYANt_pos | Cyanide transport via | Transport, | N/A | | −0.25 |
| | diffusion (extracellular to | extracellular | | | |
| | cytosol) | | | | |
| TCYNTt_pos | Thiocyanate transport via | Transport, | N/A | | −0.25 |
| | diffusion (cytosol to | extracellular | | | |
| | extracellular) | | | | |
| r1702_pos | Y+LAT2 Utilized transport | Transport, | N/A | SLC7A6 | −0.25 |
| | | extracellular | | | |
| r1762_pos | Y+LAT2 Utilized transport | Transport, | N/A | SLC7A6 | −0.25 |
| | | extracellular | | | |
| r1764_pos | Y+LAT2 Utilized transport | Transport, | N/A | SLC7A6 | −0.25 |
| | | extracellular | | | |
| r1766_pos | Y+LAT2 Utilized transport | Transport, | N/A | SLC7A6 | −0.25 |
| | | extracellular | | | |
| r1815_pos | Y+LAT2 Utilized transport | Transport, | N/A | SLC7A6 | −0.25 |
| | | extracellular | | | |
| r1817_pos | Y+LAT2 Utilized transport | Transport, | N/A | SLC7A6 | −0.25 |
| | | extracellular | | | |
| r1819_pos | Y+LAT2 Utilized transport | Transport, | N/A | SLC7A6 | −0.25 |
| | | extracellular | | | |
| r1763_pos | Y+LAT2 Utilized transport | Transport, | N/A | SLC7A6 | −0.25 |
| | | extracellular | | | |
| r1765_pos | Y+LAT2 Utilized transport | Transport, | N/A | SLC7A6 | −0.25 |
| | | extracellular | | | |
| r1807_pos | Y+LAT2 Utilized transport | Transport, | N/A | SLC7A6 | −0.25 |
| | | extracellular | | | |
| r1809_pos | Y+LAT2 Utilized transport | Transport, | N/A | SLC7A6 | −0.25 |
| | | extracellular | | | |
| r1808_pos | Y+LAT2 Utilized transport | Transport, | N/A | SLC7A6 | −0.25 |
| | | extracellular | | | |
| r1810_pos | Y+LAT2 Utilized transport | Transport, | N/A | SLC7A6 | −0.25 |
| | | extracellular | | | |
| r1696_pos | Y+LAT2 Utilized transport | Transport, | N/A | SLC7A6 | −0.25 |
| | | extracellular | | | |
| r1726_pos | Y+LAT2 Utilized transport | Transport, | N/A | SLC7A6 | −0.25 |
| | | extracellular | | | |
| r1801_pos | Y+LAT2 Utilized transport | Transport, | N/A | SLC7A6 | −0.25 |
| | | extracellular | | | |
| r1877_pos | Y+LAT2 Utilized transport | Transport, | N/A | SLC7A6 | −0.25 |
| | | extracellular | | | |
| r1700_pos | Y+LAT2 Utilized transport | Transport, | N/A | SLC7A6 | −0.25 |
| | | extracellular | | | |
| r1711_pos | Y+LAT2 Utilized transport | Transport, | N/A | SLC7A6 | −0.25 |
| | | extracellular | | | |
| r1715_pos | Y+LAT2 Utilized transport | Transport, | N/A | SLC7A6 | −0.25 |
| | | extracellular | | | |
| r1730_pos | Y+LAT2 Utilized transport | Transport, | N/A | SLC7A6 | −0.25 |
| | | extracellular | | | |
| r1771_pos | Y+LAT2 Utilized transport | Transport, | N/A | SLC7A6 | −0.25 |
| | | extracellular | | | |
| r1775_pos | Y+LAT2 Utilized transport | Transport, | N/A | SLC7A6 | −0.25 |
| | | extracellular | | | |
| r1805_pos | Y+LAT2 Utilized transport | Transport, | N/A | SLC7A6 | −0.25 |
| | | extracellular | | | |
| r1816_pos | Y+LAT2 Utilized transport | Transport, | N/A | SLC7A6 | −0.25 |
| | | extracellular | | | |
| r1820_pos | Y+LAT2 Utilized transport | Transport, | N/A | SLC7A6 | −0.25 |
| | | extracellular | | | |
| r1880_pos | Y+LAT2 Utilized transport | Transport, | N/A | SLC7A6 | −0.25 |
| | | extracellular | | | |
| r1837_pos | Y+LAT2 Utilized transport | Transport, | N/A | SLC7A6 | −0.25 |
| | | extracellular | | | |
| r1841_pos | Y+LAT2 Utilized transport | Transport, | N/A | SLC7A6 | −0.25 |
| | | extracellular | | | |
| r1846_pos | Y+LAT2 Utilized transport | Transport, | N/A | SLC7A6 | −0.25 |
| | | extracellular | | | |
| r1848_pos | Y+LAT2 Utilized transport | Transport, | N/A | SLC7A6 | −0.25 |
| | | extracellular | | | |
| r1851_pos | Y+LAT2 Utilized transport | Transport, | N/A | SLC7A6 | −0.25 |
| | | extracellular | | | |
| r1705_pos | Y+LAT2 Utilized transport | Transport, | N/A | SLC7A6 | −0.25 |
| | | extracellular | | | |
| r1710_pos | Y+LAT2 Utilized transport | Transport, | N/A | SLC7A6 | −0.25 |
| | | extracellular | | | |
| r1713_pos | Y+LAT2 Utilized transport | Transport, | N/A | SLC7A6 | −0.25 |
| | | extracellular | | | |
| r1716_pos | Y+LAT2 Utilized transport | Transport, | N/A | SLC7A6 | −0.25 |
| | | extracellular | | | |
| r1741_pos | Y+LAT2 Utilized transport | Transport, | N/A | SLC7A6 | −0.25 |
| | | extracellular | | | |
| r1745_pos | Y+LAT2 Utilized transport | Transport, | N/A | SLC7A6 | −0.25 |
| | | extracellular | | | |
| r1748_pos | Y+LAT2 Utilized transport | Transport, | N/A | SLC7A6 | −0.25 |
| | | extracellular | | | |
| r1750_pos | Y+LAT2 Utilized transport | Transport, | N/A | SLC7A6 | −0.25 |
| | | extracellular | | | |
| r1756_pos | Y+LAT2 Utilized transport | Transport, | N/A | SLC7A6 | −0.25 |
| | | extracellular | | | |
| r1760_pos | Y+LAT2 Utilized transport | Transport, | N/A | SLC7A6 | −0.25 |
| | | extracellular | | | |
| r1778_pos | Y+LAT2 Utilized transport | Transport, | N/A | SLC7A6 | −0.25 |
| | | extracellular | | | |
| r1780_pos | Y+LAT2 Utilized transport | Transport, | N/A | SLC7A6 | −0.25 |
| | | extracellular | | | |
| r1786_pos | Y+LAT2 Utilized transport | Transport, | N/A | SLC7A6 | −0.25 |
| | | extracellular | | | |
| r1790_pos | Y+LAT2 Utilized transport | Transport, | N/A | SLC7A6 | −0.25 |
| | | extracellular | | | |
| r1847_pos | Y+LAT2 Utilized transport | Transport, | N/A | SLC7A6 | −0.25 |
| | | extracellular | | | |
| r1850_pos | Y+LAT2 Utilized transport | Transport, | N/A | SLC7A6 | −0.25 |
| | | extracellular | | | |
| r1862_pos | Y+LAT2 Utilized transport | Transport, | N/A | SLC7A6 | −0.25 |
| | | extracellular | | | |
| r1865_pos | Y+LAT2 Utilized transport | Transport, | N/A | SLC7A6 | −0.25 |
| | | extracellular | | | |
| r1882_pos | Y+LAT2 Utilized transport | Transport, | N/A | SLC7A6 | −0.25 |
| | | extracellular | | | |
| r1883_pos | Y+LAT2 Utilized transport | Transport, | N/A | SLC7A6 | −0.25 |
| | | extracellular | | | |
| r1884_pos | Y+LAT2 Utilized transport | Transport, | N/A | SLC7A6 | −0.25 |
| | | extracellular | | | |
| r1885_pos | Y+LAT2 Utilized transport | Transport, | N/A | SLC7A6 | −0.25 |
| | | extracellular | | | |
| r1886_pos | Y+LAT2 Utilized transport | Transport, | N/A | SLC7A6 | −0.25 |
| | | extracellular | | | |
| r1890_pos | Y+LAT2 Utilized transport | Transport, | N/A | SLC7A6 | −0.25 |
| | | extracellular | | | |
| r1891_pos | Y+LAT2 Utilized transport | Transport, | N/A | SLC7A6 | −0.25 |
| | | extracellular | | | |
| r1892_pos | Y+LAT2 Utilized transport | Transport, | N/A | SLC7A6 | −0.25 |
| | | extracellular | | | |
| r1893_pos | Y+LAT2 Utilized transport | Transport, | N/A | SLC7A6 | −0.25 |
| | | extracellular | | | |
| r1894_pos | Y+LAT2 Utilized transport | Transport, | N/A | SLC7A6 | −0.25 |
| | | extracellular | | | |
| r1895_pos | Y+LAT2 Utilized transport | Transport, | N/A | SLC7A6 | −0.25 |
| | | extracellular | | | |
| r1896_pos | Y+LAT2 Utilized transport | Transport, | N/A | SLC7A6 | −0.25 |
| | | extracellular | | | |
| r1907_pos | Y+LAT2 Utilized transport | Transport, | N/A | SLC7A6 | −0.25 |
| | | extracellular | | | |
| r1911_pos | Y+LAT2 Utilized transport | Transport, | N/A | SLC7A6 | −0.25 |
| | | extracellular | | | |
| r1720_pos | Y+LAT2 Utilized transport | Transport, | N/A | SLC7A6 | −0.25 |
| | | extracellular | | | |
| r1871_pos | Y+LAT2 Utilized transport | Transport, | N/A | SLC7A6 | −0.25 |
| | | extracellular | | | |
| r1690_pos | Y+LAT2 Utilized transport | Transport, | N/A | SLC7A6 | −0.25 |
| | | extracellular | | | |
| r1732_pos | Y+LAT2 Utilized transport | Transport, | N/A | SLC7A6 | −0.25 |
| | | extracellular | | | |
| r1733_pos | Y+LAT2 Utilized transport | Transport, | N/A | SLC7A6 | −0.25 |
| | | extracellular | | | |
| r1734_pos | Y+LAT2 Utilized transport | Transport, | N/A | SLC7A6 | −0.25 |
| | | extracellular | | | |
| r1735_pos | Y+LAT2 Utilized transport | Transport, | N/A | SLC7A6 | −0.25 |
| | | extracellular | | | |
| r1742_pos | Y+LAT2 Utilized transport | Transport, | N/A | SLC7A6 | −0.25 |
| | | extracellular | | | |
| r1744_pos | Y+LAT2 Utilized transport | Transport, | N/A | SLC7A6 | −0.25 |
| | | extracellular | | | |
| r1852_pos | Y+LAT2 Utilized transport | Transport, | N/A | SLC7A6 | −0.25 |
| | | extracellular | | | |
| r1853_pos | Y+LAT2 Utilized transport | Transport, | N/A | SLC7A6 | −0.25 |
| | | extracellular | | | |
| r1854_pos | Y+LAT2 Utilized transport | Transport, | N/A | SLC7A6 | −0.25 |
| | | extracellular | | | |
| r1855_pos | Y+LAT2 Utilized transport | Transport, | N/A | SLC7A6 | −0.25 |
| | | extracellular | | | |
| r1863_pos | Y+LAT2 Utilized transport | Transport, | N/A | SLC7A6 | −0.25 |
| | | extracellular | | | |
| r1864_pos | Y+LAT2 Utilized transport | Transport, | N/A | SLC7A6 | −0.25 |
| | | extracellular | | | |
| r1897_pos | Y+LAT2 Utilized transport | Transport, | N/A | SLC7A6 | −0.25 |
| | | extracellular | | | |
| r1898_pos | Y+LAT2 Utilized transport | Transport, | N/A | SLC7A6 | −0.25 |
| | | extracellular | | | |
| r1899_pos | Y+LAT2 Utilized transport | Transport, | N/A | SLC7A6 | −0.25 |
| | | extracellular | | | |
| r1900_pos | Y+LAT2 Utilized transport | Transport, | N/A | SLC7A6 | −0.25 |
| | | extracellular | | | |
| r1908_pos | Y+LAT2 Utilized transport | Transport, | N/A | SLC7A6 | −0.25 |
| | | extracellular | | | |
| r1910_pos | Y+LAT2 Utilized transport | Transport, | N/A | SLC7A6 | −0.25 |
| | | extracellular | | | |
| r1739_pos | Y+LAT2 Utilized transport | Transport, | N/A | SLC7A6 | −0.25 |
| | | extracellular | | | |
| r1743_pos | Y+LAT2 Utilized transport | Transport, | N/A | SLC7A6 | −0.25 |
| | | extracellular | | | |
| r1746_pos | Y+LAT2 Utilized transport | Transport, | N/A | SLC7A6 | −0.25 |
| | | extracellular | | | |
| r1856_pos | Y+LAT2 Utilized transport | Transport, | N/A | SLC7A6 | −0.25 |
| | | extracellular | | | |
| r1860_pos | Y+LAT2 Utilized transport | Transport, | N/A | SLC7A6 | −0.25 |
| | | extracellular | | | |
| r1861_pos | Y+LAT2 Utilized transport | Transport, | N/A | SLC7A6 | −0.25 |
| | | extracellular | | | |
| r1866_pos | Y+LAT2 Utilized transport | Transport, | N/A | SLC7A6 | −0.25 |
| | | extracellular | | | |
| r1901_pos | Y+LAT2 Utilized transport | Transport, | N/A | SLC7A6 | −0.25 |
| | | extracellular | | | |
| r1905_pos | Y+LAT2 Utilized transport | Transport, | N/A | SLC7A6 | −0.25 |
| | | extracellular | | | |
| r1906_pos | Y+LAT2 Utilized transport | Transport, | N/A | SLC7A6 | −0.25 |
| | | extracellular | | | |
| r1909_pos | Y+LAT2 Utilized transport | Transport, | N/A | SLC7A6 | −0.25 |
| | | extracellular | | | |
| r1796_pos | Y+LAT2 Utilized transport | Transport, | N/A | SLC7A6 | −0.25 |
| | | extracellular | | | |
| r1835_pos | Y+LAT2 Utilized transport | Transport, | N/A | SLC7A6 | −0.25 |
| | | extracellular | | | |
| r1833_pos | Y+LAT2 Utilized transport | Transport, | N/A | SLC7A6 | −0.25 |
| | | extracellular | | | |
| r1836_pos | Y+LAT2 Utilized transport | Transport, | N/A | SLC7A6 | −0.25 |
| | | extracellular | | | |
| r1747_pos | Y+LAT2 Utilized transport | Transport, | N/A | SLC7A6 | −0.25 |
| | | extracellular | | | |
| r1749_pos | Y+LAT2 Utilized transport | Transport, | N/A | SLC7A6 | −0.25 |
| | | extracellular | | | |
| r1751_pos | Y+LAT2 Utilized transport | Transport, | N/A | SLC7A6 | −0.25 |
| | | extracellular | | | |
| r1754_pos | Y+LAT2 Utilized transport | Transport, | N/A | SLC7A6 | −0.25 |
| | | extracellular | | | |
| r1755_pos | Y+LAT2 Utilized transport | Transport, | N/A | SLC7A6 | −0.25 |
| | | extracellular | | | |
| r1757_pos | Y+LAT2 Utilized transport | Transport, | N/A | SLC7A6 | −0.25 |
| | | extracellular | | | |
| r1758_pos | Y+LAT2 Utilized transport | Transport, | N/A | SLC7A6 | −0.25 |
| | | extracellular | | | |
| r1759_pos | Y+LAT2 Utilized transport | Transport, | N/A | SLC7A6 | −0.25 |
| | | extracellular | | | |
| r1761_pos | Y+LAT2 Utilized transport | Transport, | N/A | SLC7A6 | −0.25 |
| | | extracellular | | | |
| r1777_pos | Y+LAT2 Utilized transport | Transport, | N/A | SLC7A6 | −0.25 |
| | | extracellular | | | |
| r1779_pos | Y+LAT2 Utilized transport | Transport, | N/A | SLC7A6 | −0.25 |
| | | extracellular | | | |
| r1781_pos | Y+LAT2 Utilized transport | Transport, | N/A | SLC7A6 | −0.25 |
| | | extracellular | | | |
| r1784_pos | Y+LAT2 Utilized transport | Transport, | N/A | SLC7A6 | −0.25 |
| | | extracellular | | | |
| r1785_pos | Y+LAT2 Utilized transport | Transport, | N/A | SLC7A6 | −0.25 |
| | | extracellular | | | |
| r1787_pos | Y+LAT2 Utilized transport | Transport, | N/A | SLC7A6 | −0.25 |
| | | extracellular | | | |
| r1788_pos | Y+LAT2 Utilized transport | Transport, | N/A | SLC7A6 | −0.25 |
| | | extracellular | | | |
| r1789_pos | Y+LAT2 Utilized transport | Transport, | N/A | SLC7A6 | −0.25 |
| | | extracellular | | | |
| r1791_pos | Y+LAT2 Utilized transport | Transport, | N/A | SLC7A6 | −0.25 |
| | | extracellular | | | |
| r1831_pos | Y+LAT2 Utilized transport | Transport, | N/A | SLC7A6 | −0.25 |
| | | extracellular | | | |
| r1830_pos | Y+LAT2 Utilized transport | Transport, | N/A | SLC7A6 | −0.25 |
| | | extracellular | | | |
| r1826_pos | Y+LAT2 Utilized transport | Transport, | N/A | SLC7A6 | −0.25 |
| | | extracellular | | | |
| r1823_pos | Y+LAT2 Utilized transport | Transport, | N/A | SLC7A6 | −0.25 |
| | | extracellular | | | |
| r1825_pos | Y+LAT2 Utilized transport | Transport, | N/A | SLC7A6 | −0.25 |
| | | extracellular | | | |
| r1822_pos | Y+LAT2 Utilized transport | Transport, | N/A | SLC7A6 | −0.25 |
| | | extracellular | | | |
| r1824_pos | Y+LAT2 Utilized transport | Transport, | N/A | SLC7A6 | −0.25 |
| | | extracellular | | | |
| r1832_pos | Y+LAT2 Utilized transport | Transport, | N/A | SLC7A6 | −0.25 |
| | | extracellular | | | |
| r1834_pos | Y+LAT2 Utilized transport | Transport, | N/A | SLC7A6 | −0.25 |
| | | extracellular | | | |
| r1814_pos | Y+LAT2 Utilized transport | Transport, | N/A | SLC7A6 | −0.24 |
| | | extracellular | | | |
| r1685_pos | Y+LAT2 Utilized transport | Transport, | N/A | SLC7A6 | −0.24 |
| | | extracellular | | | |
| r1707_pos | Y+LAT2 Utilized transport | Transport, | N/A | SLC7A6 | −0.24 |
| | | extracellular | | | |
| r1887_pos | Y+LAT2 Utilized transport | Transport, | N/A | SLC7A6 | −0.24 |
| | | extracellular | | | |
| r1888_pos | Y+LAT2 Utilized transport | Transport, | N/A | SLC7A6 | −0.24 |
| | | extracellular | | | |
| r1889_pos | Y+LAT2 Utilized transport | Transport, | N/A | SLC7A6 | −0.24 |
| | | extracellular | | | |
| r1708_pos | Y+LAT2 Utilized transport | Transport, | N/A | SLC7A6 | −0.24 |
| | | extracellular | | | |
| r1842_pos | Y+LAT2 Utilized transport | Transport, | N/A | SLC7A6 | −0.24 |
| | | extracellular | | | |
| r1843_pos | Y+LAT2 Utilized transport | Transport, | N/A | SLC7A6 | −0.24 |
| | | extracellular | | | |
| r1844_pos | Y+LAT2 Utilized transport | Transport, | N/A | SLC7A6 | −0.24 |
| | | extracellular | | | |
| r1737_pos | Y+LAT2 Utilized transport | Transport, | N/A | SLC7A6 | −0.24 |
| | | extracellular | | | |
| r1858_pos | Y+LAT2 Utilized transport | Transport, | N/A | SLC7A6 | −0.24 |
| | | extracellular | | | |
| r1903_pos | Y+LAT2 Utilized transport | Transport, | N/A | SLC7A6 | −0.24 |
| | | extracellular | | | |
| r1738_pos | Y+LAT2 Utilized transport | Transport, | N/A | SLC7A6 | −0.24 |
| | | extracellular | | | |
| r1859_pos | Y+LAT2 Utilized transport | Transport, | N/A | SLC7A6 | −0.24 |
| | | extracellular | | | |
| r1904_pos | Y+LAT2 Utilized transport | Transport, | N/A | SLC7A6 | −0.24 |
| | | extracellular | | | |
| r1736_pos | Y+LAT2 Utilized transport | Transport, | N/A | SLC7A6 | −0.24 |
| | | extracellular | | | |
| r1857_pos | Y+LAT2 Utilized transport | Transport, | N/A | SLC7A6 | −0.24 |
| | | extracellular | | | |
| r1902_pos | Y+LAT2 Utilized transport | Transport, | N/A | SLC7A6 | −0.24 |
| | | extracellular | | | |
| r1706_pos | Y+LAT2 Utilized transport | Transport, | N/A | SLC7A6 | −0.24 |
| | | extracellular | | | |
| r1812_pos | Y+LAT2 Utilized transport | Transport, | N/A | SLC7A6 | −0.24 |
| | | extracellular | | | |
| r1829_pos | Y+LAT2 Utilized transport | Transport, | N/A | SLC7A6 | −0.24 |
| | | extracellular | | | |
| r1752_pos | Y+LAT2 Utilized transport | Transport, | N/A | SLC7A6 | −0.24 |
| | | extracellular | | | |
| r1753_pos | Y+LAT2 Utilized transport | Transport, | N/A | SLC7A6 | −0.24 |
| | | extracellular | | | |
| r1767_pos | Y+LAT2 Utilized transport | Transport, | N/A | SLC7A6 | −0.24 |
| | | extracellular | | | |
| r1782_pos | Y+LAT2 Utilized transport | Transport, | N/A | SLC7A6 | −0.24 |
| | | extracellular | | | |
| r1783_pos | Y+LAT2 Utilized transport | Transport, | N/A | SLC7A6 | −0.24 |
| | | extracellular | | | |
| r1828_pos | Y+LAT2 Utilized transport | Transport, | N/A | SLC7A6 | −0.24 |
| | | extracellular | | | |
| r1827_pos | Y+LAT2 Utilized transport | Transport, | N/A | SLC7A6 | −0.24 |
| | | extracellular | | | |
| PHEMEe_pos | release of heme into the | Transport, | N/A | MFSD7B; | −0.24 |
| | blood | extracellular | | SLC46A1 | |
| PHEMEt_pos | heme transport | Transport, | N/A | LRP1; | −0.24 |
| | | extracellular | | SLC46A1 | |
| EX_anth(e)_pos | Exchange of anthranilate | Exchange/ | N/A | | −0.24 |
| | | demand | | | |
| | | reaction | | | |
| ANTHte_neg | Transport of anthranilate | Transport, | N/A | | −0.24 |
| | | extracellular | | | |
| KYN_pos | kynureninase | Tryptophan | 3.7.1.3 | KYNU | −0.24 |
| | | metabolism | | | |
| LFORKYNHYD_pos | L-Formylkynurenine | Tryptophan | 3.7.1.3 | KYNU | −0.24 |
| | hydrolase | metabolism | | | |
| r0239_pos | N-Formylanthranilate | Tryptophan | 3.5.1.9 | AFMID | −0.24 |
| | amidohydrolase Tryptophan | metabolism | | | |
| | metabolism EC: 3.5.1.9 | | | | |
| r1687_pos | Y+LAT2 Utilized transport | Transport, | N/A | SLC7A6 | −0.24 |
| | | extracellular | | | |
| r1717_pos | Y+LAT2 Utilized transport | Transport, | N/A | SLC7A6 | −0.24 |
| | | extracellular | | | |
| r1769_pos | Y+LAT2 Utilized transport | Transport, | N/A | SLC7A6 | −0.24 |
| | | extracellular | | | |
| r1770_pos | Y+LAT2 Utilized transport | Transport, | N/A | SLC7A6 | −0.24 |
| | | extracellular | | | |
| r1772_pos | Y+LAT2 Utilized transport | Transport, | N/A | SLC7A6 | −0.24 |
| | | extracellular | | | |
| r1773_pos | Y+LAT2 Utilized transport | Transport, | N/A | SLC7A6 | −0.24 |
| | | extracellular | | | |
| r1774_pos | Y+LAT2 Utilized transport | Transport, | N/A | SLC7A6 | −0.24 |
| | | extracellular | | | |
| r1776_pos | Y+LAT2 Utilized transport | Transport, | N/A | SLC7A6 | −0.24 |
| | | extracellular | | | |
| r1811_pos | Y+LAT2 Utilized transport | Transport, | N/A | SLC7A6 | −0.24 |
| | | extracellular | | | |
| r1818_pos | Y+LAT2 Utilized transport | Transport, | N/A | SLC7A6 | −0.24 |
| | | extracellular | | | |
| r1821_pos | Y+LAT2 Utilized transport | Transport, | N/A | SLC7A6 | −0.24 |
| | | extracellular | | | |
| r1867_pos | Y+LAT2 Utilized transport | Transport, | N/A | SLC7A6 | −0.24 |
| | | extracellular | | | |
| r1965_pos | Y+LAT2 Utilized transport | Transport, | N/A | SLC7A6 | −0.24 |
| | | extracellular | | | |
| EX_asp_L(e)_neg | L-Aspartate exchange | Exchange/ | N/A | | −0.24 |
| | | demand | | | |
| | | reaction | | | |
| EX_4abutn(e)_neg | Exchange of 4- | Exchange/ | N/A | | −0.23 |
| | ammoniobutanal | demand | | | |
| | | reaction | | | |
| EX_CE1935(e)_neg | spermine monoaldehyde | Exchange/ | N/A | | −0.23 |
| | exchange | demand | | | |
| | | reaction | | | |
| EX_CE1939(e)_neg | spermidine monoaldehyde 1 | Exchange/ | N/A | | −0.23 |
| | exchange | demand | | | |
| | | reaction | | | |
| EX_CE1940(e)_neg | spermidine monoaldehyde 2 | Exchange/ | N/A | | −0.23 |
| | exchange | demand | | | |
| | | reaction | | | |
| EX_ptrc(e)_neg | Exchange of 1,4- | Exchange/ | N/A | | −0.23 |
| | butanediammonium | demand | | | |
| | | reaction | | | |
| EX_spmd(e)_neg | Exchange of spermidine(3+) | Exchange/ | N/A | | −0.23 |
| | | demand | | | |
| | | reaction | | | |
| r0281_neg | Putrescine: oxygen | Methionine | 1.4.3.6 | AOC1 | −0.23 |
| | oxidoreductase | and | | | |
| | (deaminating) Urea cycle and | cysteine | | | |
| | metabolism of amino groups | metabolism | | | |
| | EC: 1.4.3.6 | | | | |
| r1678_pos | Y+LAT2 Utilized transport | Transport, | N/A | SLC7A6 | −0.23 |
| | | extracellular | | | |
| r1676_pos | Y+LAT2 Utilized transport | Transport, | N/A | SLC7A6 | −0.23 |
| | | extracellular | | | |
| RE1530M_neg | RE1530 | Pyrimidine | 2.7.1.21 | TK2 | −0.23 |
| | | synthesis | | | |
| NDPK8m_pos | nucleoside-diphosphate | Nucleotide | 2.7.4.6 | NME4; | −0.23 |
| | kinase (ATP: dADP), | interconversion | | NME6 | |
| | mitochondrial | | | | |
| DNDPt6m_pos | dATP transport via dGDP | Transport, | N/A | SLC25A19 | −0.23 |
| | antiport | mitochondrial | | | |
| DTMPKm_pos | dTMP kinase in mitochondria | Pyrimidine | N/A | DTYMK | −0.23 |
| | | synthesis | | | |
| D3AIBTm_neg | D-3-Amino- | Pyrimidine | 2.6.1.40 | AGXT2 | −0.22 |
| | isobutanoate: pyruvate | catabolism | | | |
| | aminotransferase, | | | | |
| | mitochondrial | | | | |
| r0483_pos | (R)-3-Amino-2- | Pyrimidine | 2.6.1.22 | ABAT | −0.22 |
| | methylpropanoate: 2- | catabolism | | | |
| | oxoglutarate | | | | |
| | aminotransferase EC: 2.6.1.22 | | | | |
| r0081_pos | L-Alanine: 2-oxoglutarate | Citric acid | 2.6.1.2 | GPT; GPT2 | −0.22 |
| | aminotransferase Glutamate | cycle | | | |
| | metabolism/Alanine and | | | | |
| | aspartate metabolism | | | | |
| | EC: 2.6.1.2 | | | | |
| r0483_neg | (R)-3-Amino-2- | Pyrimidine | 2.6.1.22 | ABAT | −0.22 |
| | methylpropanoate: 2- | catabolism | | | |
| | oxoglutarate | | | | |
| | aminotransferase EC: 2.6.1.22 | | | | |
| D3AIBTm_pos | D-3-Amino- | Pyrimidine | 2.6.1.40 | AGXT2 | −0.22 |
| | isobutanoate: pyruvate | catabolism | | | |
| | aminotransferase, | | | | |
| | mitochondrial | | | | |
| r0081_neg | L-Alanine: 2-oxoglutarate | Citric acid | 2.6.1.2 | GPT; GPT2 | −0.22 |
| | aminotransferase Glutamate | cycle | | | |
| | metabolism/Alanine and | | | | |
| | aspartate metabolism | | | | |
| | EC: 2.6.1.2 | | | | |
| r1672_pos | Y+LAT2 Utilized transport | Transport, | N/A | SLC7A6 | −0.22 |
| | | extracellular | | | |
| r1674_pos | Y+LAT2 Utilized transport | Transport, | N/A | SLC7A6 | −0.22 |
| | | extracellular | | | |
| r1792_pos | Y+LAT2 Utilized transport | Transport, | N/A | SLC7A6 | −0.22 |
| | | extracellular | | | |
| r1995_pos | Amino Acid-Polyamine- | Transport, | N/A | SLC7A9 | −0.22 |
| | Organocation (APC) | extracellular | | | |
| | TCDB: 2.A.3.8.15 | | | | |
| HISyLATtc_pos | Transport of L-Histidine by | Transport, | N/A | SLC3A2; | −0.22 |
| | y+LAT1 or y+LAT2 | extracellular | | SLC7A6; | |
| | transporters in small | | | SLC7A7 | |
| | intestine and kidney | | | | |
| ARGNm_pos | arginase (m) | Urea cycle | 3.5.3.1 | ARG2 | −0.22 |
| UREAtm_neg | Urea transport via diffusion | Urea cycle | N/A | AQP9 | −0.22 |
| FKYNH_pos | N-Formyl-L-kynurenine | Tryptophan | 3.5.1.9 | AFMID | −0.22 |
| | amidohydrolase | metabolism | | | |
| TRPO2_pos | L-Tryptophan: oxygen 2,3- | Tryptophan | 1.13.11.11 | IDO1; IDO2; | −0.22 |
| | oxidoreductase (decyclizing) | metabolism | | TDO2 | |
| EX_tcynt(e)_pos | Thiocyanate exchange | Exchange/ | N/A | | −0.22 |
| | | demand | | | |
| | | reaction | | | |
| r1993_pos | Amino Acid-Polyamine- | Transport, | N/A | SLC7A9 | −0.22 |
| | Organocation (APC) | extracellular | | | |
| | TCDB: 2.A.3.8.15 | | | | |
| r1994_pos | Amino Acid-Polyamine- | Transport, | N/A | SLC7A9 | −0.22 |
| | Organocation (APC) | extracellular | | | |
| | TCDB: 2.A.3.8.15 | | | | |
| r1996_pos | Amino Acid-Polyamine- | Transport, | N/A | SLC7A9 | −0.22 |
| | Organocation (APC) | extracellular | | | |
| | TCDB: 2.A.3.8.15 | | | | |
| r2008_pos | Amino Acid-Polyamine- | Transport, | N/A | SLC7A9 | −0.22 |
| | Organocation (APC) | extracellular | | | |
| | TCDB: 2.A.3.8.15 | | | | |

| | patho_manual_{—} | patho_manual_{—} | patho_manual_c.only_{—} | patho_manual_c.only_{—} |
|---|---|---|---|---|
| | c.cellPaper_{—} | c.cellPaper_{—} | Th17p.cellPaper_{—} | Th17p.cellPaper_{—} |
| rxn | vitro_48 hr_GFP | vivo | vitro_48 hr_GFP | vivo |
| EX_C02470(e)_pos | −0.10 | 0.01 | 0.01 | −0.03 |
| r1030_pos | −0.10 | 0.01 | 0.01 | −0.03 |
| KYN3OX_pos | −0.10 | 0.01 | 0.01 | −0.03 |
| RE2349C_pos | −0.10 | 0.01 | 0.01 | −0.03 |
| EX_gthrd(e)_neg | 0.17 | −0.18 | −0.06 | 0.04 |
| EX_CE2011(e)_pos | −0.34 | 0.33 | 0.04 | 0.23 |
| RE0702E_pos | −0.34 | 0.33 | 0.04 | 0.23 |
| r1725_pos | −0.12 | 0.06 | −0.04 | −0.03 |
| r1728_pos | −0.12 | 0.06 | −0.04 | −0.03 |
| r1731_pos | −0.12 | 0.06 | −0.04 | −0.03 |
| r1876_pos | −0.12 | 0.06 | −0.04 | −0.03 |
| r1879_pos | −0.12 | 0.06 | −0.04 | −0.03 |
| r1881_pos | −0.12 | 0.06 | −0.04 | −0.03 |
| r1695_pos | −0.12 | 0.05 | −0.04 | −0.03 |
| r1698_pos | −0.12 | 0.05 | −0.04 | −0.03 |
| r1701_pos | −0.12 | 0.05 | −0.04 | −0.03 |
| r1800_pos | −0.12 | 0.05 | −0.04 | −0.06 |
| r1803_pos | −0.12 | 0.05 | −0.04 | −0.06 |
| r1806_pos | −0.12 | 0.05 | −0.04 | −0.06 |
| HPYRDC_pos | −0.02 | 0.08 | −0.12 | −0.03 |
| r1799_pos | −0.14 | 0.06 | −0.05 | −0.05 |
| r1722_pos | −0.14 | 0.06 | −0.05 | −0.03 |
| r1723_pos | −0.14 | 0.06 | −0.05 | −0.03 |
| r1873_pos | −0.14 | 0.06 | −0.05 | −0.03 |
| r1874_pos | −0.14 | 0.06 | −0.05 | −0.03 |
| r1798_pos | −0.14 | 0.05 | −0.05 | −0.06 |
| r1683_pos | −0.14 | 0.04 | −0.05 | −0.06 |
| r1686_pos | −0.14 | 0.04 | −0.05 | −0.06 |
| r1677_pos | −0.16 | 0.04 | −0.06 | −0.06 |
| r1721_pos | −0.14 | 0.03 | −0.05 | −0.03 |
| r1872_pos | −0.14 | 0.03 | −0.05 | −0.03 |
| r1693_pos | −0.14 | 0.03 | −0.05 | −0.04 |
| r1692_pos | −0.14 | 0.03 | −0.05 | −0.04 |
| r1797_pos | −0.14 | 0.01 | −0.05 | −0.04 |
| r1691_pos | −0.14 | 0.01 | −0.05 | −0.03 |
| EX_cyan(e)_neg | −0.12 | 0.33 | 0.02 | 0.25 |
| MCPST_pos | −0.12 | 0.33 | 0.02 | 0.25 |
| CYANt_pos | −0.12 | 0.33 | 0.02 | 0.25 |
| TCYNTt_pos | −0.12 | 0.33 | 0.02 | 0.25 |
| r1702_pos | −0.09 | 0.14 | 0.03 | 0.07 |
| r1762_pos | −0.09 | 0.14 | 0.03 | 0.07 |
| r1764_pos | −0.09 | 0.14 | 0.03 | 0.07 |
| r1766_pos | −0.09 | 0.14 | 0.01 | 0.07 |
| r1815_pos | −0.09 | 0.14 | 0.01 | 0.07 |
| r1817_pos | −0.09 | 0.14 | 0.01 | 0.07 |
| r1819_pos | −0.09 | 0.14 | 0.01 | 0.07 |
| r1763_pos | −0.11 | 0.13 | −0.03 | 0.05 |
| r1765_pos | −0.11 | 0.13 | −0.03 | 0.05 |
| r1807_pos | −0.11 | 0.13 | −0.03 | 0.05 |
| r1809_pos | −0.11 | 0.13 | −0.03 | 0.05 |
| r1808_pos | −0.12 | 0.13 | −0.04 | 0.05 |
| r1810_pos | −0.12 | 0.13 | −0.04 | 0.05 |
| r1696_pos | −0.12 | 0.07 | −0.04 | −0.02 |
| r1726_pos | −0.12 | 0.07 | −0.04 | −0.02 |
| r1801_pos | −0.12 | 0.07 | −0.04 | −0.02 |
| r1877_pos | −0.12 | 0.07 | −0.04 | −0.02 |
| r1700_pos | −0.12 | 0.07 | −0.04 | −0.03 |
| r1711_pos | −0.12 | 0.07 | −0.04 | −0.03 |
| r1715_pos | −0.12 | 0.07 | −0.04 | −0.03 |
| r1730_pos | −0.12 | 0.07 | −0.04 | −0.03 |
| r1771_pos | −0.12 | 0.07 | −0.04 | −0.03 |
| r1775_pos | −0.12 | 0.07 | −0.04 | −0.03 |
| r1805_pos | −0.12 | 0.07 | −0.04 | −0.03 |
| r1816_pos | −0.12 | 0.07 | −0.04 | −0.03 |
| r1820_pos | −0.12 | 0.07 | −0.04 | −0.03 |
| r1880_pos | −0.12 | 0.07 | −0.04 | −0.03 |
| r1837_pos | −0.12 | 0.06 | −0.04 | −0.05 |
| r1841_pos | −0.12 | 0.06 | −0.04 | −0.05 |
| r1846_pos | −0.12 | 0.06 | −0.04 | −0.05 |
| r1848_pos | −0.12 | 0.06 | −0.04 | −0.05 |
| r1851_pos | −0.12 | 0.06 | −0.04 | −0.05 |
| r1705_pos | −0.12 | 0.06 | −0.04 | −0.05 |
| r1710_pos | −0.12 | 0.06 | −0.04 | −0.05 |
| r1713_pos | −0.12 | 0.06 | −0.04 | −0.05 |
| r1716_pos | −0.12 | 0.06 | −0.04 | −0.05 |
| r1741_pos | −0.12 | 0.06 | −0.04 | −0.04 |
| r1745_pos | −0.12 | 0.06 | −0.04 | −0.04 |
| r1748_pos | −0.12 | 0.06 | −0.04 | −0.04 |
| r1750_pos | −0.12 | 0.06 | −0.04 | −0.04 |
| r1756_pos | −0.12 | 0.06 | −0.04 | −0.04 |
| r1760_pos | −0.12 | 0.06 | −0.04 | −0.04 |
| r1778_pos | −0.12 | 0.06 | −0.04 | −0.04 |
| r1780_pos | −0.12 | 0.06 | −0.04 | −0.04 |
| r1786_pos | −0.12 | 0.06 | −0.04 | −0.04 |
| r1790_pos | −0.12 | 0.06 | −0.04 | −0.04 |
| r1847_pos | −0.12 | 0.06 | −0.04 | −0.04 |
| r1850_pos | −0.12 | 0.06 | −0.04 | −0.04 |
| r1862_pos | −0.12 | 0.06 | −0.04 | −0.04 |
| r1865_pos | −0.12 | 0.06 | −0.04 | −0.04 |
| r1882_pos | −0.12 | 0.06 | −0.04 | −0.04 |
| r1883_pos | −0.12 | 0.06 | −0.04 | −0.04 |
| r1884_pos | −0.12 | 0.06 | −0.04 | −0.04 |
| r1885_pos | −0.12 | 0.06 | −0.04 | −0.04 |
| r1886_pos | −0.12 | 0.06 | −0.04 | −0.04 |
| r1890_pos | −0.12 | 0.06 | −0.04 | −0.04 |
| r1891_pos | −0.12 | 0.06 | −0.04 | −0.04 |
| r1892_pos | −0.12 | 0.06 | −0.04 | −0.04 |
| r1893_pos | −0.12 | 0.06 | −0.04 | −0.04 |
| r1894_pos | −0.12 | 0.06 | −0.04 | −0.04 |
| r1895_pos | −0.12 | 0.06 | −0.04 | −0.04 |
| r1896_pos | −0.12 | 0.06 | −0.04 | −0.04 |
| r1907_pos | −0.12 | 0.06 | −0.04 | −0.04 |
| r1911_pos | −0.12 | 0.06 | −0.04 | −0.04 |
| r1720_pos | −0.12 | 0.06 | −0.04 | −0.03 |
| r1871_pos | −0.12 | 0.06 | −0.04 | −0.03 |
| r1690_pos | −0.12 | 0.05 | −0.04 | −0.03 |
| r1732_pos | −0.12 | 0.05 | −0.04 | −0.07 |
| r1733_pos | −0.12 | 0.05 | −0.04 | −0.07 |
| r1734_pos | −0.12 | 0.05 | −0.04 | −0.07 |
| r1735_pos | −0.12 | 0.05 | −0.04 | −0.07 |
| r1742_pos | −0.12 | 0.05 | −0.04 | −0.07 |
| r1744_pos | −0.12 | 0.05 | −0.04 | −0.07 |
| r1852_pos | −0.12 | 0.05 | −0.04 | −0.07 |
| r1853_pos | −0.12 | 0.05 | −0.04 | −0.07 |
| r1854_pos | −0.12 | 0.05 | −0.04 | −0.07 |
| r1855_pos | −0.12 | 0.05 | −0.04 | −0.07 |
| r1863_pos | −0.12 | 0.05 | −0.04 | −0.07 |
| r1864_pos | −0.12 | 0.05 | −0.04 | −0.07 |
| r1897_pos | −0.12 | 0.05 | −0.04 | −0.07 |
| r1898_pos | −0.12 | 0.05 | −0.04 | −0.07 |
| r1899_pos | −0.12 | 0.05 | −0.04 | −0.07 |
| r1900_pos | −0.12 | 0.05 | −0.04 | −0.07 |
| r1908_pos | −0.12 | 0.05 | −0.04 | −0.07 |
| r1910_pos | −0.12 | 0.05 | −0.04 | −0.07 |
| r1739_pos | −0.12 | 0.05 | −0.04 | −0.08 |
| r1743_pos | −0.12 | 0.05 | −0.04 | −0.08 |
| r1746_pos | −0.12 | 0.05 | −0.04 | −0.08 |
| r1856_pos | −0.12 | 0.05 | −0.04 | −0.08 |
| r1860_pos | −0.12 | 0.05 | −0.04 | −0.08 |
| r1861_pos | −0.12 | 0.05 | −0.04 | −0.08 |
| r1866_pos | −0.12 | 0.05 | −0.04 | −0.08 |
| r1901_pos | −0.12 | 0.05 | −0.04 | −0.08 |
| r1905_pos | −0.12 | 0.05 | −0.04 | −0.08 |
| r1906_pos | −0.12 | 0.05 | −0.04 | −0.08 |
| r1909_pos | −0.12 | 0.05 | −0.04 | −0.08 |
| r1796_pos | −0.12 | 0.05 | −0.04 | −0.06 |
| r1835_pos | −0.12 | 0.03 | −0.04 | −0.08 |
| r1833_pos | −0.12 | 0.03 | −0.04 | −0.07 |
| r1836_pos | −0.12 | 0.03 | −0.04 | −0.07 |
| r1747_pos | −0.12 | 0.01 | −0.04 | −0.06 |
| r1749_pos | −0.12 | 0.01 | −0.04 | −0.06 |
| r1751_pos | −0.12 | 0.01 | −0.04 | −0.06 |
| r1754_pos | −0.12 | 0.01 | −0.04 | −0.06 |
| r1755_pos | −0.12 | 0.01 | −0.04 | −0.06 |
| r1757_pos | −0.12 | 0.01 | −0.04 | −0.06 |
| r1758_pos | −0.12 | 0.01 | −0.04 | −0.06 |
| r1759_pos | −0.12 | 0.01 | −0.04 | −0.06 |
| r1761_pos | −0.12 | 0.01 | −0.04 | −0.06 |
| r1777_pos | −0.12 | 0.01 | −0.04 | −0.06 |
| r1779_pos | −0.12 | 0.01 | −0.04 | −0.06 |
| r1781_pos | −0.12 | 0.01 | −0.04 | −0.06 |
| r1784_pos | −0.12 | 0.01 | −0.04 | −0.06 |
| r1785_pos | −0.12 | 0.01 | −0.04 | −0.06 |
| r1787_pos | −0.12 | 0.01 | −0.04 | −0.06 |
| r1788_pos | −0.12 | 0.01 | −0.04 | −0.06 |
| r1789_pos | −0.12 | 0.01 | −0.04 | −0.06 |
| r1791_pos | −0.12 | 0.01 | −0.04 | −0.06 |
| r1831_pos | −0.12 | 0.01 | −0.04 | −0.06 |
| r1830_pos | −0.12 | 0.01 | −0.04 | −0.09 |
| r1826_pos | −0.12 | 0.01 | −0.04 | −0.09 |
| r1823_pos | −0.12 | 0.01 | −0.04 | −0.07 |
| r1825_pos | −0.12 | 0.01 | −0.04 | −0.07 |
| r1822_pos | −0.12 | 0.00 | −0.04 | −0.08 |
| r1824_pos | −0.12 | 0.00 | −0.04 | −0.08 |
| r1832_pos | −0.12 | 0.00 | −0.04 | −0.08 |
| r1834_pos | −0.12 | 0.00 | −0.04 | −0.08 |
| r1814_pos | −0.09 | 0.14 | 0.01 | 0.07 |
| r1685_pos | −0.13 | 0.06 | −0.04 | −0.04 |
| r1707_pos | −0.12 | 0.06 | −0.04 | −0.05 |
| r1887_pos | −0.12 | 0.06 | −0.04 | −0.04 |
| r1888_pos | −0.12 | 0.06 | −0.04 | −0.04 |
| r1889_pos | −0.12 | 0.06 | −0.04 | −0.04 |
| r1708_pos | −0.14 | 0.06 | −0.05 | −0.03 |
| r1842_pos | −0.12 | 0.05 | −0.04 | −0.05 |
| r1843_pos | −0.12 | 0.05 | −0.04 | −0.05 |
| r1844_pos | −0.12 | 0.05 | −0.04 | −0.05 |
| r1737_pos | −0.12 | 0.05 | −0.04 | −0.08 |
| r1858_pos | −0.12 | 0.05 | −0.04 | −0.08 |
| r1903_pos | −0.12 | 0.05 | −0.04 | −0.08 |
| r1738_pos | −0.12 | 0.04 | −0.04 | −0.08 |
| r1859_pos | −0.12 | 0.04 | −0.04 | −0.08 |
| r1904_pos | −0.12 | 0.04 | −0.04 | −0.08 |
| r1736_pos | −0.12 | 0.03 | −0.04 | −0.05 |
| r1857_pos | −0.12 | 0.03 | −0.04 | −0.05 |
| r1902_pos | −0.12 | 0.03 | −0.04 | −0.05 |
| r1706_pos | −0.14 | 0.03 | −0.05 | −0.03 |
| r1812_pos | −0.12 | 0.01 | −0.04 | −0.04 |
| r1829_pos | −0.12 | −0.01 | −0.04 | −0.07 |
| r1752_pos | −0.14 | −0.02 | −0.06 | −0.06 |
| r1753_pos | −0.14 | −0.02 | −0.06 | −0.06 |
| r1767_pos | −0.14 | −0.02 | −0.06 | −0.06 |
| r1782_pos | −0.14 | −0.02 | −0.06 | −0.06 |
| r1783_pos | −0.14 | −0.02 | −0.06 | −0.06 |
| r1828_pos | −0.12 | −0.03 | −0.04 | −0.10 |
| r1827_pos | −0.12 | −0.05 | −0.04 | −0.06 |
| PHEMEe_pos | −0.42 | 0.09 | 0.09 | 0.12 |
| PHEMEt_pos | −0.42 | 0.00 | 0.09 | 0.00 |
| EX_anth(e)_pos | −0.28 | 0.06 | −0.08 | −0.05 |
| ANTHte_neg | −0.28 | 0.06 | −0.08 | −0.05 |
| KYN_pos | −0.28 | 0.06 | −0.08 | −0.05 |
| LFORKYNHYD_pos | −0.28 | 0.06 | −0.08 | −0.05 |
| r0239_pos | −0.28 | 0.06 | −0.08 | −0.05 |
| r1687_pos | −0.10 | 0.12 | 0.05 | 0.05 |
| r1717_pos | −0.10 | 0.12 | 0.05 | 0.05 |
| r1769_pos | −0.10 | 0.12 | 0.05 | 0.05 |
| r1770_pos | −0.10 | 0.12 | 0.05 | 0.05 |
| r1772_pos | −0.10 | 0.12 | 0.05 | 0.05 |
| r1773_pos | −0.10 | 0.12 | 0.05 | 0.05 |
| r1774_pos | −0.10 | 0.12 | 0.05 | 0.05 |
| r1776_pos | −0.10 | 0.12 | 0.05 | 0.05 |
| r1811_pos | −0.10 | 0.12 | 0.05 | 0.05 |
| r1818_pos | −0.10 | 0.12 | 0.05 | 0.05 |
| r1821_pos | −0.10 | 0.12 | 0.05 | 0.05 |
| r1867_pos | −0.10 | 0.12 | 0.05 | 0.05 |
| r1965_pos | −0.19 | 0.01 | −0.05 | −0.03 |
| EX_asp_L(e)_neg | −0.02 | 0.23 | −0.06 | 0.13 |
| EX_4abutn(e)_neg | −0.75 | 0.00 | 0.05 | 0.00 |
| EX_CE1935(e)_neg | −0.75 | 0.00 | 0.05 | 0.00 |
| EX_CE1939(e)_neg | −0.75 | 0.00 | 0.05 | 0.00 |
| EX_CE1940(e)_neg | −0.75 | 0.00 | 0.05 | 0.00 |
| EX_ptrc(e)_neg | −0.75 | 0.00 | 0.05 | 0.00 |
| EX_spmd(e)_neg | −0.75 | 0.00 | 0.05 | 0.00 |
| r0281_neg | −0.75 | 0.00 | 0.05 | 0.00 |
| r1678_pos | −0.16 | 0.03 | −0.06 | −0.04 |
| r1676_pos | −0.16 | 0.01 | −0.06 | −0.03 |
| RE1530M_neg | 0.07 | 0.19 | −0.12 | 0.06 |
| NDPK8m_pos | 0.22 | 0.16 | −0.12 | 0.07 |
| DNDPt6m_pos | 0.24 | 0.15 | −0.10 | 0.05 |
| DTMPKm_pos | 0.24 | 0.14 | −0.10 | 0.06 |
| D3AIBTm_neg | −0.05 | −0.18 | 0.06 | −0.21 |
| r0483_pos | −0.05 | −0.18 | 0.06 | −0.21 |
| r0081_pos | −0.05 | −0.18 | 0.06 | −0.19 |
| r0483_neg | −0.05 | −0.18 | 0.06 | −0.21 |
| D3AIBTm_pos | −0.05 | −0.19 | 0.06 | −0.21 |
| r0081_neg | −0.05 | −0.19 | 0.06 | −0.21 |
| r1672_pos | −0.10 | 0.12 | 0.05 | 0.05 |
| r1674_pos | −0.10 | 0.12 | 0.05 | 0.05 |
| r1792_pos | −0.10 | 0.12 | 0.05 | 0.05 |
| r1995_pos | −0.10 | 0.21 | 0.05 | 0.23 |
| HISyLATtc_pos | −0.09 | 0.09 | 0.09 | 0.01 |
| ARGNm_pos | −0.19 | 0.24 | −0.14 | 0.21 |
| UREAtm_neg | −0.19 | 0.24 | −0.14 | 0.21 |
| FKYNH_pos | −0.25 | 0.06 | −0.12 | −0.05 |
| TRPO2_pos | −0.25 | 0.06 | −0.12 | −0.05 |
| EX_tcynt(e)_pos | −0.10 | 0.33 | 0.05 | 0.25 |
| r1993_pos | −0.10 | 0.21 | 0.06 | 0.18 |
| r1994_pos | −0.10 | 0.21 | 0.06 | 0.18 |
| r1996_pos | −0.10 | 0.21 | 0.06 | 0.18 |
| r2008_pos | −0.10 | 0.21 | 0.06 | 0.18 |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of shifting T cell balance in a population of cells comprising T cells, said method comprising contacting the population of cells with one or more agents capable of modulating the polyamine pathway.

2. The method of claim 1, wherein Th17 cell balance is shifted towards Treg-like cells and/or is shifted away from Th17 cells; or is shifted towards Th17 cells and/or is shifted away from Treg-like cells.

3. The method of claim 1, wherein Th17 cell balance is shifted towards non-pathogenic Th17 cells and/or is shifted away from pathogenic Th17 cells; or is shifted towards pathogenic Th17 cells and/or is shifted away from non-pathogenic Th17 cells.

4. The method of any of claims 1 to 3, wherein the one or more agents capable of shifting T cell balance towards Treg-like cells and/or away from Th17 cells comprise a polyamine or polyamine analogue.

5. The method of claim 4, wherein the polyamine analogue is 2-(difluoromethyl)ornithine (DFMO) or a derivative thereof.

6. The method of any of claims 1 to 3, wherein the one or more agents modulate the expression, activity or function of one or more proteins in the polyamine pathway or downstream targets thereof.

7. The method of claim 6, wherein the one or more agents modulate the expression, activity or function of SAT1.

8. The method of claim 7, wherein the one or more agents comprise Diminazene-aceturate or a derivative thereof.

9. The method of claim 6, wherein the one or more agents modulate the expression, activity or function of ODC1.

10. The method of claim 9, wherein the one or more agents comprise DFMO or a derivative thereof.

11. The method of claim 6, wherein the one or more agents modulate the expression, activity or function of spermidine synthase (SRM).

12. The method of claim 11, wherein the one or more agents comprise trans-4-methylcyclohexylamine (MCHA) or a derivative thereof.

13. The method of claim 6, wherein the one or more agents modulate the expression, activity or function of spermine synthase (SMS).

14. The method of claim 11, wherein the one or more agents comprise N-(3-aminopropyl)-cyclohexylamine (APCHA) or a derivative thereof.

15. The method of claim 6, wherein the one or more agents modulate the expression, activity or function of one or more genes or gene products selected from the group consisting of JMJD3, POU2F2, POU2F1, POU5F1B, POU3F4, POU1F1, POU3F2, POU3F3, POU4F2, POU2F3, POU3F1, POU4F1, NFAT5, NFATC2, c-MAF and BATF.

16. The method of claim 15, wherein the one or more agents capable of shifting T cell balance towards Th17 cells and/or away from Treg-like cells comprises GSK-J1.

17. The method of claim 15, wherein the one or more agents capable of shifting T cell balance towards Treg-like cells and/or away from Th17 cells comprises an agonist of JMJD3.

18. The method of any of claims 6 to 17, wherein the one or more agents comprise a small molecule, small molecule degrader, genetic modifying agent, antibody, antibody fragment, antibody-like protein scaffold, aptamer, protein, or any combination thereof.

19. The method of claim 18, wherein the genetic modifying agent comprises a CRISPR system, RNAi system, zinc finger nuclease system, TALE system, or a meganuclease.

20. The method of claim 19, wherein the CRISPR system is a Class 1 or Class 2 CRISPR system.

21. The method of claim 20, wherein the Class 2 system comprises a Type II Cas polypeptide.

22. The method of claim 21, wherein the Type II Cas is a Cas9.

23. The method of claim 20, wherein the Class 2 system comprises a Type V Cas polypeptide.

24. The method of claim 23, wherein the Type V Cas is Cas12a, Cas12b, Cas12c, Cas12d (CasY), Cas12e(CasX), or Cas14.

25. The method of claim 20, wherein the Class 2 system comprises a Type VI Cas polypeptide.

26. The method of claim 25, wherein the Type VI Cas is Cas13a, Cas13b, Cas13c or Cas13d.

27. The method of any of claims 20 to 26, wherein the CRISPR system comprises a dCas fused or otherwise linked to a nucleotide deaminase.

28. The method of claim 27, wherein the nucleotide deaminase is a cytidine deaminase or an adenosine deaminase.

29. The method of claim 20, wherein the CRISPR system is a prime editing system.

30. The method of any of claims 1 to 29, wherein the population of cells comprises naïve T cells, Th1 cells and/or Th17 cells.

31. The method of any of claims 1 to 30, wherein the population of cells are in vitro cells.

32. The method of any of claims 1 to 30, wherein the population of cells is an in vivo population of cells in a subject at risk for or suffering from a disease or condition characterized by an aberrant immune response, whereby the one or more agents are used to treat the disease or condition.

33. The method of any of claims 1 to 30, wherein the population of cells are ex vivo cells obtained from a healthy donor subject or from a subject at risk for or suffering from a disease or condition characterized by an aberrant immune response.

34. The method of claim 32 or 33, wherein the disease is an inflammatory and/or autoimmune disorder.

35. The method of claim 34, wherein the inflammatory disorder is selected from the group consisting of Multiple Sclerosis (MS), Irritable Bowel Disease (IBD), Crohn's disease, ulcerative colitis, spondyloarthritides, Systemic Lupus Erythematosus (SLE), Vitiligo, rheumatoid arthritis, psoriasis, Sjögren's syndrome, diabetes, psoriasis, Irritable bowel syndrome (IBS), allergic asthma, food allergies and rheumatoid arthritis.

36. The method of claim 32 or 33, wherein the condition is an autoimmune response.

37. The method of claim 36, wherein the subject at risk for or suffering from an autoimmune response is a subject undergoing immunotherapy.

38. The method of claim 37, wherein the immunotherapy is checkpoint blockade therapy and/or adoptive cell transfer.

39. The method of claim 38, wherein the checkpoint blockade therapy comprises anti-PD1, anti-CTLA4, anti-PD-L1, anti-TIM3, anti-TIGIT, anti-LAG3, or combinations thereof.

40. The method of any of claims 37 to 39, wherein the one or more agents are administered before, during or after administering the immunotherapy.

41. The method of any of claims 37 to 40, wherein the subject undergoing immunotherapy is suffering from cancer.

42. The method of any of claims 30 to 41, wherein the naïve T cells are differentiated into Th17 cells, Th1 cells and/or Treg cells.

43. The method of claim 42, wherein the one or more agents are administered to the population of cells during differentiation.

44. The method of claim 42 or 43, wherein the differentiation conditions comprise cell culture media supplemented with IL-6 and TGF-β1 or supplemented with IL-1β, IL-6 and IL-23.

45. The method of claim 42 or 43, wherein T cell differentiation is shifted towards Treg cells and/or is shifted away from Th17 cells.

46. The method of claim 42 or 43, wherein T cell differentiation is shifted towards Th17 cells and/or is shifted away from Treg cells.

47. The method of claim 42 or 43, wherein T cell differentiation is shifted towards Th1 cells and/or is shifted away from Th17 cells.

48. The method of claim 42 or 43, wherein T cell differentiation is shifted towards Th17 cells and/or is shifted away from Th1 cells.

49. The method of claim 42 or 43, wherein T cell differentiation is shifted towards non-pathogenic Th17 cells and/or is shifted away from pathogenic Th17 cells.

50. A population of T cells obtained by the method according to any of claims 1 to 49.

51. A pharmaceutical composition comprising the population of T cells according to claim 50.

52. A method of treating a disease or condition characterized by an aberrant immune response comprising administering the pharmaceutical composition of claim 51 to a subject in need thereof.

53. A method of monitoring Th17 mediated autoimmunity in a subject comprising detecting one or more polyamines in the subject, wherein increased polyamines as compared to a control indicates autoimmunity.

54. A method of treating autoimmunity in a subject in need thereof, comprising monitoring Th17 mediated autoimmunity in the subject according to claim 53; and treating the subject according to any of claims 1 to 52 when increased polyamines are detected.

55. A method of shifting Th17 cell pathogenicity in a population of cells comprising T cells, said method comprising contacting the population of cells with one or more agents capable of modulating a reaction of the glycolysis pathway according to Table 1 or 2.

56. The method of claim 55, wherein the one or more agents modulate expression, activity, or function of a gene or gene product selected from the group consisting of: PGAM, G6PD, PKM, Aldo, PFKM, TA, G6PC, GK, ENO1, PCK1, TPI1, PGK1, GAPDHS, PDHA1, and GPD1.

57. The method of claim 56, wherein the one or more agents is selected from the group consisting of: EGCG, 2,5-Anhydro-D-glucitol-1,6-diphosphate, S-HD-CoA, DHEA, TX1, Gimeracil, Shikonin, Pyruvate Kinase Inhibitor III, 2,3-Dihydroxypropyl dichloroacetate (DCA), 2,9-Dimethyl-BC, Koningic acid, CBR-470-1, SF2312, PhAh, ENOblock, 3-MPA, and 6,8-Bis(benzylthio)octanoic acid.

58. The method of claim 55 or 56, wherein the one or more agents comprise a small molecule, small molecule degrader, genetic modifying agent, antibody, antibody fragment, antibody-like protein scaffold, aptamer, protein, or any combination thereof.

59. The method of claim 58, wherein the genetic modifying agent comprises a CRISPR system, RNAi system, zinc finger nuclease system, TALE system, or a meganuclease.

60. The method of claim 59, wherein the CRISPR system is a Class 1 or Class 2 CRISPR system.

61. The method of claim 60, wherein the Class 2 system comprises a Type II Cas polypeptide.

62. The method of claim 61, wherein the Type II Cas is a Cas9.

63. The method of claim 60, wherein the Class 2 system comprises a Type V Cas polypeptide.

64. The method of claim 63, wherein the Type V Cas is Cas12a, Cas12b, Cas12c, Cas12d (CasY), Cas12e(CasX), or Cas14.

65. The method of claim 60, wherein the Class 2 system comprises a Type VI Cas polypeptide.

66. The method of claim 65, wherein the Type VI Cas is Cas13a, Cas13b, Cas13c or Cas13d.

67. The method of any of claims 60 to 66, wherein the CRISPR system comprises a dCas fused or otherwise linked to a nucleotide deaminase.

68. The method of claim 67, wherein the nucleotide deaminase is a cytidine deaminase or an adenosine deaminase.

69. The method of claim 60, wherein the CRISPR system is a prime editing system.

70. The method of any of claims 55 to 69, wherein the population of cells comprises naïve T cells, Th1 cells and/or Th17 cells.

71. The method of any of claims 55 to 70, wherein the population of cells are in vitro cells.

72. The method of any of claims 55 to 70, wherein the population of cells is an in vivo population of cells in a subject at risk for or suffering from a disease or condition characterized by an aberrant immune response, whereby the one or more agents are used to treat the disease or condition.

73. The method of any of claims 55 to 70, wherein the population of cells are ex vivo cells obtained from a healthy donor subject or from a subject at risk for or suffering from a disease or condition characterized by an aberrant immune response.

74. The method of claim 72 or 73, wherein the disease is an inflammatory and/or autoimmune disorder.

75. The method of claim 74, wherein the inflammatory disorder is selected from the group consisting of Multiple Sclerosis (MS), Irritable Bowel Disease (IBD), Crohn's disease, ulcerative colitis, spondyloarthritides, Systemic Lupus Erythematosus (SLE), Vitiligo, rheumatoid arthritis, psoriasis, Sjögren's syndrome, diabetes, psoriasis, Irritable bowel syndrome (IBS), allergic asthma, food allergies and rheumatoid arthritis.

76. The method of claim 72 or 73, wherein the condition is an autoimmune response.

77. The method of claim 76, wherein the subject at risk for or suffering from an autoimmune response is a subject undergoing immunotherapy.

78. The method of claim 77, wherein the immunotherapy is checkpoint blockade therapy and/or adoptive cell transfer.

79. The method of claim 78, wherein the checkpoint blockade therapy comprises anti-PD1, anti-CTLA4, anti-PD-L1, anti-TIM3, anti-TIGIT, anti-LAG3, or combinations thereof.

80. The method of any of claims 77 to 79, wherein the one or more agents are administered before, during or after administering the immunotherapy.

81. The method of any of claims 77 to 80, wherein the subject undergoing immunotherapy is suffering from cancer.

82. The method of any of claim 70, 71 or 73, wherein the naïve T cells are differentiated into Th17 cells.

83. The method of claim 82, wherein the one or more agents are administered to the population of cells during differentiation.

84. The method of claim 82 or 83, wherein the differentiation conditions comprise cell culture media supplemented with IL-6 and TGF-β1 or supplemented with IL-1β, IL-6 and IL-23.

85. The method of any of claims 82 to 84, wherein T cell differentiation is shifted towards non-pathogenic Th17 cells and/or is shifted away from pathogenic Th17 cells.

86. A population of T cells obtained by the method according to any of claims 55 to 85.

87. A pharmaceutical composition comprising the population of T cells according to claim 86.

88. A method of treating a disease or condition characterized by an aberrant immune response comprising administering the pharmaceutical composition of claim 87 to a subject in need thereof.

89. A data driven method of detecting metabolic target genes and pathways, comprising:
providing single cell RNA-seq reads obtained from a population of cells or an RNA library from multiple cells, wherein each single cell represents an observation, and the number of observations allows statistical power to discern statistically significant metabolic targets;
providing metabolic data comprising the metabolic reactions in the population of cells; and
simulating the metabolic fluxes at the single-cell level by projecting the transcriptomic profile onto the metabolic network, thereby producing a quantitative metabolic profile of each cell.

90. The method of claim 89, wherein spatial, temporal or lineage delineated RNA-seq data is used to identify the metabolic state in single cells across a tissue, over time or in a cell lineage.

91. The method of claim 89, wherein the method comprises treating a population of cells with a drug for use in identifying metabolic adaptation to the drug.

92. The method of claim 89, wherein the method comprises predicting targets that will shift a population of cells in one state to another state.

93. The method of claim 92, wherein the state is shifted:
towards Treg-like cells and/or is shifted away from Th17 cells; or
towards Th17 cells and/or is shifted away from Treg-like cells; or
towards non-pathogenic Th17 cells and/or is shifted away from pathogenic Th17 cells; or
towards pathogenic Th17 cells and/or is shifted away from non-pathogenic Th17 cells.

94. The method of claim 89, wherein the method is used to determine resistance to a drug, wherein the method comprises determining metabolic pathways modulated in resistant subjects as compared to sensitive subjects.

95. The method of claim 89, wherein the method comprises analyzing single cells obtained from a diseased tissue for use in determining metabolic shifts in disease.

96. The method of claim 95, wherein the disease comprises a degenerative disease, cancer, a metabolic disease, aging, autoimmunity or inflammation.

97. The method of claim 96, wherein the disease comprises cardiovascular disease.

98. The method of claim 96, wherein the disease comprises diabetes.

99. The method of any of claims 89 to 95, wherein the single cells comprise cells from an animal, plant, or bacteria.

100. The method of claim 89, wherein the method comprises identifying metabolic shifts in a host cell contacted with a microbiome.


SEQUENCE LISTING (22 sequences)

SEQ ID NO: 1 - protein, 7 aa, Simian virus 40
PKKKRKV

SEQ ID NO: 2 - protein, 10 aa, Simian virus 40
PKKKRKVEAS

SEQ ID NO: 3 - protein, 16 aa, Artificial sequence
  Synthetic
KRPAATKKAGQAKKKK

SEQ ID NO: 4 - protein, 9 aa, Homo sapiens
PAAKRVKLD

SEQ ID NO: 5 - protein, 11 aa, Homo sapiens
RQRRNELKRSP

SEQ ID NO: 6 - protein, 38 aa, Artificial sequence
  Synthetic
NQSSNFGPMKGGNFGGRSSGPYGGGGQYFAKPRNQGGY

SEQ ID NO: 7 - protein, 42 aa, Artificial sequence
  Synthetic
RMRIZFKNKGKDTAELRRRRVEVSVELRKAKKDEQILKRRNV

SEQ ID NO: 8 - protein, 8 aa, Artificial sequence
  Synthetic
VSRKRPRP

SEQ ID NO: 9 - protein, 8 aa, Artificial sequence
  Synthetic
PPKKARED

SEQ ID NO: 10 - protein, 8 aa, Homo sapiens
PQPKKKPL

SEQ ID NO: 11 - protein, 12 aa, Mus musculus
SALIKKKKKMAP

SEQ ID NO: 12 - protein, 5 aa, Influenza virus
DRLRR

SEQ ID NO: 13 - protein, 7 aa, Influenza virus
PKQKKRK

SEQ ID NO: 14 - protein, 10 aa, Hepatitis virus
RKLKKKIKKL

SEQ ID NO: 15 - protein, 10 aa, Mus musculus
REKKKFLKRR

SEQ ID NO: 16 - protein, 20 aa, Homo sapiens
KRKGDEVDGVDEVAKKKSKK

SEQ ID NO: 17 - protein, 17 aa, Homo sapiens
RKCLQAGMNLEARKTKK

SEQ ID NO: 18 - protein, 288 aa, Artificial sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 19 - protein, 183 aa, Artificial sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 20 - protein, 107 aa, Artificial sequence
  Synthetic
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 21 - protein, 9 aa, Homo sapiens
IEVMYPPPY

SEQ ID NO: 22 - protein, 107 aa, Homo sapiens; identical to SEQ ID NO: 20